(19) United States
(12) Patent Application Publication
(10) Pub. No.: US 2025/0182844 A1
(43) Pub. Date: Jun. 5, 2025

(54) Methods for Identifying Shared Biological Pathways Between Diseases Using Mendelian Randomization

(71) Applicant: AMPEL BioSolutions, LLC, Charlottesville, VA (US)

(72) Inventors: Jessica KAIN, Charlottesville, VA (US); Katherine A. OWEN, Charlottesville, VA (US); Amrie C. GRAMMER, Charlottesville, VA (US); Peter E. LIPSKY, Charlottesville, VA (US)

(21) Appl. No.: 18/937,448

(22) Filed: Nov. 5, 2024

Related U.S. Application Data
(63) Continuation of application No. PCT/US2023/021260, filed on May 5, 2023.
(60) Provisional application No. 63/407,567, filed on Sep. 16, 2022.
(60) Provisional application No. 63/339,874, filed on May 9, 2022.
(60) Provisional application No. 63/339,285, filed on May 6, 2022.

(51) Int. Cl. G16B 20/00; G16B 40/20; G16B 45/00; G16H 50/30

(52) U.S. Cl. CPC G16B 20/00; G16B 40/20; G16B 45/00; G16H 50/30

(57) ABSTRACT

The present disclosure provides methods for determining biological pathways and nucleotide polymorphisms (NPs) shared between two diseases.

### CROSS-REFERENCE

The present application is a continuation of PCT/US2023/021260, filed May 5, 2023, which claims the benefit of U.S. Provisional Application No. 63/339,285 filed May 6, 2022, U.S. Provisional Application No. 63/339,874 filed May 9, 2022, and U.S. Provisional Application No. 63/407,567 filed Sep. 16, 2022, the contents of which are hereby incorporated by reference in their entireties.

### BACKGROUND

Many diseases, including Systemic Lupus Erythematosus (SLE), are heterogeneous in nature, and have variable causation, course and responsiveness to therapy. Coronary artery disease (CAD) is a leading cause of death in patients with SLE, however, genetic association between SLE and CAD, and biological pathways likely involved in both pathologies remain unknown. There is a need for understanding biological pathways involved in the pathogenesis of these conditions to allow identification and optimization of therapies.

### SUMMARY

The present disclosure provides methods for identifying shared biological pathways between two diseases, e.g., genetic networks, that may be involved in pathogenesis of either or both diseases. Understanding of such shared biological pathways can help in determining disease status in patients, including whether patients having one disease or disorder may have or be at risk of developing a second disease or disorder. This information also can be used to identify personalized treatment strategies. One aspect of the method includes clustering nucleotide polymorphisms (NPs) associated with one disease, according to the biological pathways associated with the NPs, to obtain NP clusters, and performing causal analysis of the NP clusters on a second disease using a grouped-NP Mendelian randomization (MR) analysis. Performing causal analysis with NP clusters that were built according to genetic networks, allows measuring causal effect of genetic networks associated with one disease, separately on a second disease. MR is generally employed to test for causal relationships between phenotypes of interest, and prior to the present disclosure have not been applied for understanding causal relations of NPs clustered by biological pathway. Methods described herein can also identify NPs, such as single nucleotide polymorphisms (SNPs), and/or genes, associated with the shared biological pathways. As described in a non-limiting manner in Example 1, shared biological pathways, SNPs and genes between lupus and Coronary artery disease (CAD) were identified by clustering SNPs associated with lupus according to the biological pathways associated with the SNPs, and performing the causal analysis of the SNP clusters on CAD using a grouped-SNP MR analysis. In some embodiments, the shared biological pathways, shared NPs, and shared genes between the first disease and the second disease are respectively biological pathways, NPs, and genes that are associated with the first disease and are positive causal or negative causal on the second disease. In some embodiments, the shared biological pathways, shared NPs, and shared genes between the first disease and the second disease are respectively biological pathways, NPs, and genes that are associated with the first disease and are positive causal on the second disease.

One aspect of the present disclosure is directed to a method for determining shared biological pathways and/or shared nucleotide polymorphisms (NPs) between a first disease and a second disease. The method can also identify biological pathways associated with the shared NPs. The method can include any one of, any combination of, or all of steps (a)-(f). Step (a) can include selecting a first set of nucleotide polymorphisms (NPs) associated with the first disease from a first dataset. The first dataset can contain data regarding association of a first plurality of NPs with the first disease. Step (b) can include mapping one or more NPs of the first set of NPs selected in step (a) to genes, to identify a plurality of NP-mapped genes. Step (c) can include clustering the plurality of NP-mapped genes to obtain one or more gene clusters. Step (d) can include clustering the one or more NPs mapped in step (b), to obtain a first set of NP clusters. Step (e) can include performing a causal inference analysis to select a subset of NP clusters from the first set of NP clusters obtained in step (d). In certain embodiments, each NP cluster within the subset of NP clusters, has a positive or negative causal effect on the second disease, wherein the subset of NP clusters selected in step (e) includes NP clusters having positive causal effect on the second disease, and/or NP clusters having negative causal effect on the second disease. In certain embodiments, each NP cluster within the subset of NP clusters has a positive causal effect on the second disease. In certain embodiments, each NP cluster within the subset of NP clusters has a negative causal effect on the second disease. Step (f) can include functionally annotating i) one or more NP cluster of the subset of NP clusters selected in step (e) and/or ii) gene clusters mapped with the one or more NP cluster of the subset of NP clusters, thereby determining the shared biological pathways between the first and the second disease. The NPs within the NP clusters within the subset of NP clusters selected in step (e) are the shared NPs between the first disease and the second disease. For a respective NP within a NP cluster within the subset of NP clusters obtained in step (e), the biological pathway associated with the NP may be determined based on the functional annotation (e.g. as determined in step (f)) of the NP cluster, and/or of the gene cluster mapped to the NP cluster. The method can be performed in a computer.

In step (a), the first set of NPs can be selected from the first dataset based at least on the p-value for statistical significance of the association of the NPs with the first disease. In certain embodiments, the p-value for statistical significance of the association of each NP within the first set of NPs with the first disease is lower than about 1*10⁻⁶. In certain embodiments, the p-value for statistical significance of the association of each NP within the first set of NPs with the first disease is lower than about 5*10⁻⁸.

In certain embodiments, in step (b) the plurality of NP-mapped genes are identified by mapping the one or more NPs of (e.g., within) the first set of NPs to their i) associated expression quantitative trait loci (eQTL) expression genes (E-Genes), ii) associated transcription factors and downstream target genes (T-Genes), iii) associated protein coding genes (C-genes), iv) proximal genes (P-genes), or any combination thereof. The one or more NPs of the first set of NPs can be mapped to their associated E-genes, T-genes, C-genes, and/or P-genes using a suitable method, as understood by a person of ordinary skill in the art. In certain embodiments, all the NPs of the first set of NPs are mapped to genes to identify the plurality of NP-mapped genes. In certain embodiments, the NPs are single nucleotide polymorphism (SNPs), and non limiting methods for mapping SNPs to their associated E-genes, T-genes, C-genes, and/or P-genes can include a mapping method described in i) Owen et al., Analysis of trans-ancestral SLE risk loci identifies unique biologic networks and drug targets in African and European Ancestries. The American Journal of Human Genetics, 2020 107(5), 864-881; Fulco et al., Activity-by-Contact model of enhancer-promoter regulation from thousands of CRISPR perturbations. Nat Genet. 2019 51(12), 1664-1669; Nasser et al., Genome-wide enhancer maps link risk variants to disease genes. Nature 2021 593(7858), 238-243; or the like. In certain embodiments, the SNPs are mapped to the associated E-genes, T-genes, C-genes, and/or P-genes according to the mapping method described in Owen et al., Analysis of trans-ancestral SLE risk loci identifies unique biologic networks and drug targets in African and European Ancestries. The American Journal of Human Genetics, 2020 107(5), 864-881. In step (c) the plurality of NP-mapped genes identified in step (b) are clustered, wherein genes (e.g. NP-mapped genes) determined to be associated with same network of genes and/or within same biological pathway, such as within same genetic network, are grouped in the same cluster. Gene clustering can be performed based on a suitable method as understood by a person of ordinary skill in the art, including but not limited to protein-protein interactions of proteins encoded by the NP-mapped genes, gene co-expression, genetic pathway, genetic annotations, genetic associations, or any combination thereof; and can be performed using any suitable database. In certain embodiments, the plurality of NP-mapped genes are clustered based on protein-protein interactions of proteins encoded by the NP-mapped genes. In certain embodiments, protein coding NP-mapped genes are clustered based on protein-protein interactions of the proteins encoded by the protein coding NP-mapped genes. In certain embodiments, the protein-protein interactions based clustering of the NP-mapped genes includes i) clustering the encoded proteins (e.g. by the NP-mapped genes) into one or more protein clusters, and ii) clustering the NP-mapped genes to form the one or more gene clusters of step (c), based on the clustering of the encoded proteins, wherein for a respective protein cluster formed in step (c)-(i), in step (c)-(ii) a gene cluster is formed containing the genes that encodes the proteins within the respective protein cluster. Clustering of the encoded proteins, into the one or more protein clusters, e.g., as in (i) of step (c), can include grouping proteins determined to be within same biological pathway such as protein network, within same protein cluster. The encoded proteins can be clustered into the one or more protein clusters based on physical interaction and/or functional association among the encoded proteins, wherein for a respective protein cluster, each protein within the cluster is determined to be capable of physically interacting and/or functionally associated, with at least one other protein within the cluster. Without intending to be limited by theory, it is believed that, physically interacting and/or functionally associated protein may belong to the same biological pathway.

The one or more NPs mapped in step (b), can be clustered in step (d) using a suitable method, as understood by a person of ordinary skill in the art. In certain embodiments, in step (d), the one or more NPs mapped in step (b) are clustered based on the clustering of the plurality of NP-mapped genes in step (c), to obtain the first set of NP clusters. The first set of NP clusters can contain one or more NP clusters. In some embodiments, for, a gene cluster formed in step (c), in step (d) a NP cluster containing the mapped NPs to the genes of the gene cluster, is formed. In certain embodiments, the one or more NPs mapped in step (b), can be clustered in step (d) based on association between the NPs to obtain the first set of NP clusters.

The causal inference analysis of step (e) can include a causal inference method, such as a Mendelian randomization based method. The causal inference method, such as the Mendelian randomization based method of step (e) can include, determining causal effect of the NP clusters of the first set of NP clusters obtained in step (d), on the second disease, wherein the NP clusters having positive or negative causal effect on the second disease are selected to form the subset of NP clusters of step (e), e.g., the subset of NP clusters selected in step (e) include NP clusters having positive causal effect on the second disease, and/or NP clusters having negative causal effect on the second disease. For causal analysis of a respective NP-cluster of the first set of NP clusters of step (d), at least 2 NPs within the respective NP cluster are used as instrument variables (IVs). For causal analysis of a respective NP-cluster of the first set of NP clusters of step (d), at least 2 NPs within the respective NP cluster collectively are used as instrument variables (IVs), summary statistics from a second dataset is used as exposure, and a third dataset is used as outcome. The second dataset can include data regarding summary statistics of association of a second plurality of NPs with the first disease. The second data set can be same or different than the first dataset. In certain embodiments, the first data set and the second data set are same. In certain embodiments, the first data set and the second data set are different, and the first plurality of NPs overlap at least partially with the second plurality of NPs, e.g., at least a portion of the NPs listed within the first dataset are also listed in the second dataset. The third dataset can include data regarding association of a third plurality of NPs with the second disease. In certain embodiments, the third plurality of NPs overlap at least partially with the first plurality of NPs, e.g., at least a portion of the NPs listed within the first dataset are also listed in the third dataset. In certain embodiments, the third plurality of NPs overlap at least partially with the second plurality of NPs, e.g., at least a portion of the NPs listed within the second dataset are also listed in the third dataset. In certain embodiments, the overlap between the first and second plurality of NPs overlap at least partially with the third plurality of NPs, e.g., at least a portion of the NPs that are listed within both the first dataset and the second dataset are also listed in the third dataset. In certain embodiments, proxy NPs are used in place of one or more NPs from the first plurality of NPs. Proxy NPs can be NPs that are in high linkage disequilibrium, e.g. are highly correlated, with one or more NPs from the first plurality of NPs. In certain embodiments, for causal analysis of a respective NP cluster within the first set of NP clusters obtained in step (d), at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP cluster, collectively are used as IVs. In certain embodiments, for causal analysis of each NP cluster within the first set of NP clusters, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP cluster, collectively are used as IVs, wherein for different NP clusters the number of NPs used can be the same or different. The causal inference analysis of step (e) can be performed using a method as described in the Examples. For a NP cluster of the first set of NP clusters, the NPs used as IVs for the causal inference analysis of step (e), can be selected based on the i) strength of association of the NPs with the first disease, ii) the strength of association of the NPs with the second disease, iii) the strength of association of NPs with confounding traits, iv) linkage disequilibrium with other NPs, v) genomic location, vi) allele harmonization between the second and third datasets, or any combination thereof. For each NP cluster of the first set of NP clusters, the NPs used as IVs for the causal inference analysis of step (e), can be selected based independently on the i) strength of association of the NPs with the first disease, ii) the strength of association of the NPs with the second disease, iii) the strength of association of NPs with confounding traits, iv) linkage disequilibrium with other NPs, v) genomic location, vi) allele harmonization between the second and third datasets, or any combination thereof. In certain embodiments, the strength of association with the first disease of the NPs used as IVs has a threshold nominal or genome-wide significance, depending on the genotyping method and/or sample size of genetic association study. In certain embodiments, the strength of association with the first disease of the NPs used as IVs has i) a nominal significance p value <1*10\hat{ }5 or <1*10\hat{ }6, and/or ii) a genome-wide significance p-value <5*10\hat{ }8. In certain embodiments, the strength of association with the first disease of NPs used as IVs has p-value <1*10\hat{ }5 or more significant than genome-wide significance (p-value<5*10\hat{ }8). In certain embodiments, p-value for the strength of association of NPs selected as IVs, with the first disease is <1*10\hat{ }5. In certain embodiments, p-value for the strength of association of NPs selected as IVs, with the first disease is <1*10\hat{ }-6. In certain embodiments, NPs associated (e.g., p-value <1*10\hat{ }5) with the second disease are excluded from using as IVs. In certain embodiments, NPs associated (e.g., p-value <1*10\hat{ }5) with confounding traits are excluded from using as IVs. In certain embodiments, the significance of association with the second disease and/or potential confounders of NPs to be excluded from IVs can be low (e.g. p-value <1*10\hat{ }5) or reach genome-wide (p-value <₅*10\hat{ }-8) significance. The NPs used as IVs can be independent of each other. In certain embodiments, for the NPs selected/used as IVs i) p-value for the strength of association with the first disease is <1*10\hat{ }5, and ii) p-value for the strength of association with the second disease is >1*10\hat{ }-5. In certain embodiments, for causal analysis of a respective NP-cluster of the first set of NP clusters of step (d), the NPs of the respective NP cluster used as IVs have i) p-value for the strength of association with the first disease <1*10\hat{ }-5, and ii) p-value for the strength of association with the second disease >1*10\hat{ }-5. In certain embodiments, for causal analysis of a respective NP-cluster of the first set of NP clusters of step (d), NPs of the respective NP cluster used as IVs have i) p-value for the strength of association with the first disease <1*10\hat{ }5, ii) p-value for the strength of association with the second disease >1*10\hat{ }5, and/or iii) p-value for the strength of association with the confounding traits >1*10\hat{ }5. In certain embodiments, the level of correlation, or linkage disequilibrium, between the NPs used as IVs has r\hat{ }2<0.001, 0.01, 0.1, or 0.5. In certain embodiments, the level of correlation, or linkage disequilibrium, between the NPs used as IVs has r\hat{ }2<0.001. In certain embodiments, the level of correlation, or linkage disequilibrium, between the NPs used as IVs has r\hat{ }2<0.01. In certain embodiments, the level of correlation, or linkage disequilibrium, between the NPs used as IVs has r\hat{ }2<0.1. In certain embodiments, the level of correlation, or linkage disequilibrium, between the NPs used as IVs has r\hat{ }2<0.5. In certain embodiments, an independent set of NPs is obtained using the clump_data( ) function in the TwoSampleMR R package, and is used as IVs. In certain embodiments, NPs in genomic regions, such as the major histocompatibility complex (MHC) or HLA region on the short-arm of chromosome 6, that are difficult to genotype, have extensive linkage disequilibrium or pleiotropy, and/or are unreliable, are excluded from using as IVs. In certain embodiments, NPs from MHC or HLA region on the short-arm of chromosome 6 are excluded from using as IVs. In certain embodiments, allele harmonization between the second and third dataset is performed to ensure the summary statistics for the first and second disease are based on the same reference and alternative alleles for each NP used as IVs. In certain embodiments, allele harmonization can be performed using the harmonise_data( ) function in the TwoSampleMR R package. In certain embodiments, NPs used as IVs are selected based on type of NP (e.g. coding, expression, transcription factor, proximal, etc.). In certain embodiments, NPs used as IVs are selected based on type of genomic region the NP occurs in (e.g. coding gene, non-coding gene, exon, intron, untranslated regions, eQTLs, transcription factor motifs, promoters, enhances, or other regulatory elements, etc.). In certain embodiments, NPs that are mapped to multiple genes and/or assigned to multiple clusters are excluded from being IVs for specific or all clusters. NPs selection of NPs for use as IVs can depend on the type of causal inference, such MR method being used for performing the causal inference analysis of step (e). NPs selected as IVs can have any one of, any combination of or all, of the properties mentioned in the herein, such as in this paragraph.

In certain embodiments, the selection of the NP cluster in step (e) can be based on the p-value of the causal effect. In certain embodiments, the p-value for the positive or negative causal estimate on the second disease of the NP-clusters selected in the step (e) is below 0.05. In certain embodiments, the p-value for the positive or negative causal estimate of each NP cluster of the subset of NP clusters selected in step (e), on the second disease is below 0.05, wherein the subset of NP clusters contains NP clusters having positive causal effect of the second disease, and/or NP clusters having negative causal effect of the second disease. In certain embodiments, the NP-clusters selected in the step (e), has positive causal effect on the second disease. In certain embodiments, the NP-clusters selected in the step (e), has negative causal effect on the second disease. In certain embodiments, the NP-clusters selected in the step (e), has positive causal effect on the second disease, and the p-value for the positive causal estimate of each NP cluster of the subset of NP clusters selected in step (e), on the second disease is below 0.05. In certain embodiments, the NP-clusters selected in the step (e), has negative causal effect on the second disease, and the p-value for the negative causal estimate of each NP cluster of the subset of NP clusters selected in step (e), on the second disease is below 0.05. In certain embodiments, the selection of the NP cluster in step (e) can be based on a Bonferroni-corrected p-value of the causal effect. In certain embodiments, the Bonferroni-corrected p-value threshold for the positive or negative causal estimate on the second disease of the NP-clusters selected in the step (e) is 0.05/[Number of NP clusters selected]. In certain embodiments, the Bonferroni-corrected p-value threshold for the positive or negative causal estimate of each NP cluster of the subset of NP clusters selected in step (e), on the second disease is 0.05/[Number of NP clusters selected], wherein the subset of NP clusters contains NP clusters having positive causal effect of the second disease, and/or NP clusters having negative causal effect of the second disease. In certain embodiments, the Bonferroni-corrected p-value threshold for the positive or negative causal estimate on the second disease of the NP-clusters selected in the step (e) is 0.00075. In certain embodiments, the Bonferroni-corrected p-value threshold for the positive or negative causal estimate of each NP cluster of the subset of NP clusters selected in step (e), on the second disease is 0.00075, wherein the subset of NP clusters contains NP clusters having positive causal effect of the second disease, and/or NP clusters having negative causal effect of the second disease. In certain embodiments, the causal inference analysis of step (e) can be performed using a plurality of causal inference methods. In certain embodiments, the causal inference analysis of step (e) can be performed using a plurality of causal inference, such as MR based methods, and each NP-cluster selected in the step (e), has positive causal effect (e.g. based on p-value) on the second disease based on at least two causal inference based methods, or negative causal effect (e.g. based on p-value) on the second disease based on at least two causal inference methods. In certain embodiments, each NP-cluster selected in the step (e), has positive causal effect on the second disease based on at least two causal inference methods. In certain embodiments, each NP-cluster selected in the step (e), has negative causal effect on the second disease based on at least two causal inference methods. Non-limiting examples of the causal inference, such as MR based methods used in step (e) can include inverse-weighted (IVW), IVW-random effects, IVW-fixed effects, simple mode, simple mode-NOME, weighted mode, weighted mode-NOME, simple median, weighted median, penalized weighted median, two sample maximum likelihood, Maximum likehoods, RAPS, Egger, Egger-bootstrap, PRESSO-raw, PRESSO-OC, or any combination thereof.

Step (f) can include functionally annotating i) one or more NP cluster of the subset of NP clusters selected in step (e) and/or ii) gene clusters mapped with the one or more NP cluster of the subset of NP clusters. The mapped gene cluster can be a gene cluster of step (c). A gene cluster containing genes mapped (e.g., as identified in step (b)) to the NPs within a NP cluster, is mapped to the NP cluster, and vice versa. In some embodiments, in step (f), for a respective NP cluster of the subset of NP clusters and/or a respective gene cluster mapped with the respective NP cluster, the functionally annotating comprises (i) overlapping the respective mapped gene cluster (i.e., gene cluster mapped with the respective NP cluster), with one or more gene function signature lists to determine, significant overlap between the respective mapped gene cluster and the one or more gene function signature lists; and (ii) annotating the respective NP cluster and/or the respective mapped gene cluster with one or more functional characterizations, based at least on the significant overlap of the mapped gene cluster. In some embodiments, in step (f), for a respective NP cluster of the subset of NP clusters, the functionally annotating comprises (i) overlapping a gene cluster mapped with the respective NP cluster, with one or more gene function signature lists to determine, significant overlap between the mapped gene cluster (i.e., gene cluster mapped with the respective NP cluster), and the one or more gene function signature lists; and (ii) annotating the respective NP cluster with one or more functional characterizations, based at least on the significant overlap of the mapped gene cluster. In some embodiments, in step (f), for a respective gene cluster mapped with a NP cluster of the subset of NP clusters, the functionally annotating comprises (i) overlapping the respective mapped gene cluster (i.e., gene cluster mapped with the NP cluster), with one or more gene function signature lists to determine, significant overlap between the respective mapped gene cluster and the one or more gene function signature lists; and (ii) annotating the respective mapped gene cluster with one or more functional characterizations, based at least on the significant overlap of the mapped gene cluster. In some embodiments, in step (f), for a respective gene cluster mapped to a NP cluster of the subset of NP clusters, the functionally annotating comprises (i) overlapping the respective gene cluster, with one or more gene function signature lists to determine, significant overlap between the gene cluster and the one or more gene function signature lists; and (ii) annotating the respective gene cluster with one or more functional characterizations, based at least on the significant overlap of the mapped gene cluster. The functional annotations for a gene cluster can be used to interpret a NP cluster containing the NPs mapped to the genes of the gene cluster. The one or more gene function signature lists can contain curated signatures of cell types and/or biological functions. Gene function signature lists can contain of a collection of genes (represented as gene symbols) that have been statistically demonstrated using various metrics to be representative of a cell type and/or function, and genes in gene function signature lists, based on cell type and/or function are grouped into one or more functional characterization groups. The overlap, e.g., in step (f)-(i), can include categorical comparison of gene symbols in a given gene cluster, to gene symbols in a given functional characterization group of a gene function signature list. For a respective gene cluster, the categorical comparison can include findings of gene symbols in the gene cluster, within gene symbols in a gene functional characterization group. The categorical comparisons can be performed using any suitable technique. In some embodiments, the categorical comparisons is conducted using the Fisher's exact test. The significant overlap between, e.g. between a respective gene cluster and a respective functional characterization group, can have a threshold Fisher's adjusted p value. The p value used can account for biological variability. Significant overlap, between a respective gene cluster and a respective functional characterization group, can also satisfy overlap of a threshold minimum number of genes between the respective gene cluster and the respective functional characterization group. In certain embodiments, the threshold minimum number of genes are about 1 gene to about 12 genes. In certain embodiments, the threshold minimum number of genes are about 1 gene. The threshold minimum number of genes can depend on the size of the gene cluster being overlapped. In certain embodiments, the threshold minimum number of genes are about 3 genes. Once the overlapping one or more functional characterization groups, for a respective gene cluster is identified (e.g., in step (f)-(i)), in step (f)-(ii) the NP cluster that is mapped to the respective gene cluster, can be functionally annotated based on the overlapping one or more functional characterization groups. All gene clusters of step (c) may or may not be functionally annotated. All clusters of the subset of NP clusters selected in step (e) may or may not be functionally annotated. Every gene clusters mapped to NP cluster of the subset of NP clusters selected in step (e), may or may not have significant overlap. In certain embodiments, NP clusters mapped to the gene clusters having significant overlap, are functionally annotated. In certain embodiments, all clusters of the subset of NP clusters selected in step (e) are functionally annotated in step (f). In certain embodiments, the one or more gene function signature lists contain AMPEL LuGENE, AMPEL Ancestry, AMPEL Endotype.32, Endotype.kidney, AMPEL tissues (Tis), Biologically Informed Gene Clustering (BIG-C) signature, Gene Ontology (GO) database, Hallmark gene sets, KEGG Pathway Database, Reactome signature, BRETIGEA signature, IPA, EnrichR, or any combination thereof. In certain embodiments, the one or more gene function signature lists contain AMPEL LuGENE, AMPEL Ancestry, AMPEL tissues (Tis), Biologically Informed Gene Clustering (BIG-C) signature, Gene Ontology (GO) database, Ingenuity Pathway Analysis (IPA), EnrichR, or any combination thereof. In certain embodiments, the one or more gene function signature lists contain Biologically Informed Gene Clustering (BIG-C) signature. In certain embodiments, the one or more gene function signature lists contain IPA, and/or EnrichR. The gene function lists, the functional characterization groups (e.g. categories) within the list, and genes with the functional characterization groups for AMPEL Ancestry and BIG-C, are provided in Catalina, Michelle D., et al. “Patient ancestry significantly contributes to molecular heterogeneity of systemic lupus erythematosus.” JCI insight 5.15 (2020); for GO is publicly available at http://geneontology.org/; for BRETIGEA is provided in McKenzie, Andrew T., et al. “Brain cell type specific gene expression and co-expression network architectures.” Scientific reports 8.1 (2018): 1-19; for Hallmark gene sets, KEGG Pathway Database, Reactome signature is publicly available at http://www.gsea-msigdb.org/gsea/msigdb/collections.jsp. IPA is publicly available at https://www.qiagen.com/us/products/discovery-and-translational-research/next-generation-sequencing/informatics-and-data/interpretation-content-databases/ingenuity-pathway-analysis/. EnrichR is publicly available at https://maayanlab.cloud/Enrichr/. In certain embodiments, the one or more NP cluster of the subset of NP clusters selected in step (e), can be annotated using Ingenuity Pathway Analysis method available from QIAGEN.

The first disease can be an oligogenic or polygenic phenotype and/or disease. In certain embodiments, the first disease can be an autoimmune disease, a heart disease, a pulmonary disease, depression, cancer, a diabetic disease, a nonalcoholic fatty liver disease, a digestive system disease, or a kidney disease. The second disease can be a different disease from the first disease, and can be an oligogenic or polygenic phenotype and/or disease. In certain embodiments, the second disease is a different disease from the first disease, and is an autoimmune disease, a heart disease, a pulmonary disease, depression, cancer, a diabetic disease, a nonalcoholic fatty liver disease, a digestive system disease, or a kidney disease. In certain embodiments, the first disease is an autoimmune disease, and the second disease is a pulmonary disease, depression, cancer, a diabetic disease, a nonalcoholic fatty liver disease, a digestive system disease, or a kidney disease. In certain embodiments, the first disease is an autoimmune disease and the second disease is a heart disease. In certain embodiments, the autoimmune disease is lupus. In certain embodiments, the autoimmune disease is multiple sclerosis (MS). Heart disease can be coronary artery disease (CAD), cardiovascular disease, myocardial infarction, ischemic stroke, coronary atherosclerosis, or cardiomyopathy.

In certain embodiments, the first disease is lupus, coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder, inflammatory bowel disease, multiple sclerosis, or glomerulonephritis. In certain embodiment, the first disease is lupus. The second disease is different from the first disease, and is lupus, coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, multiple sclerosis, or glomerulonephritis. In certain aspects, the second disease is CAD. In certain embodiments, the first disease is lupus, and the second disease is CAD. The NPs can be single nucleotide polymorphisms (SNPs), indels, splice variants, structural variants, copy number variants, transposons, or other forms of genetic variation. In certain embodiment, the NPs are SNPs. In certain embodiments, the first disease is lupus, and the NPs are SNPs, and the method can determine shared biological pathways and/or shared SNPs between lupus and a second disease. In certain aspects, the first disease is lupus, the second disease is CAD, the NPs are SNPs, and the method can determine shared biological pathways and/or shared SNPs between lupus and CAD. Lupus can be any type of lupus including but not limited to systemic lupus erythematosus (SLE), lupus nephritis, cutaneous lupus erythematosus, drug-induced lupus, and neonatal lupus. In certain embodiments, lupus can be SLE. In certain embodiments, lupus can be lupus nephritis.

In certain embodiments, the method includes diagnosis of the first disease and/or the second disease in a patient, wherein the method comprises detecting presence of one or more of the shared NPs in a biological sample from the patient. In certain embodiments, the method includes selecting, recommending and/or administering a treatment to the patient based on the presence of the one or more shared NPs in the biological sample. The treatment can be a treatment for the first disease and/or a treatment for the second disease. In certain embodiments, the treatment targets a biological pathway associated with a shared NP detected in the biological sample.

In certain embodiments, the method includes diagnosis of the second disease in a patient, wherein the method comprises detecting presence of the one or more of the shared NPs (e.g., NPs within the shared NP clusters) in a biological sample from the patient. The patient can be determined to have the second disease, or can be determined to be at risk of developing the second disease, when the NPs detected/present in the biological sample comprises a higher number of positive causal NPs, compared to negative shared NPs in a biological sample from the patient. The patient can have the first disease. In certain embodiments, the method includes selecting, recommending and/or administering a treatment to the patient based on the presence of the one or more shared NPs in the biological sample. In certain embodiments, the method includes selecting, recommending and/or administering a treatment to the patient when the NPs detected/present in the biological sample comprises a higher number of positive causal NPs, compared to negative causal NPs in a biological sample from the patient. The treatment can be a treatment for the second disease. In certain embodiments, the treatment targets a biological pathway associated with a shared NP detected in the biological sample. In certain embodiments, the method includes diagnosis of the second disease in a patient, wherein the method comprises detecting presence of one or more of the positive causal shared NPs in a biological sample from the patient. In certain embodiments, the method includes diagnosis of the second disease in a patient, wherein the method comprises detecting presence of a higher number of positive causal NPs, compared to negative causal NPs in a biological sample from the patient. The patient can have the first disease. In certain embodiments, the method includes selecting, recommending and/or administering a treatment to the patient based on the presence of the one or more positive causal NPs in the biological sample. In certain embodiments, the method includes selecting, recommending and/or administering a treatment to the patient based on the presence of a higher number of positive causal NPs, compared to negative causal NPs in the biological sample. In certain embodiments, the method includes selecting, recommending and/or administering a treatment to the patient based on the presence of a higher number of positive causal NPs, compared to negative causal NPs in the biological sample, and patient having one or more symptoms of the second disease. The treatment can be a treatment for the second disease. In certain embodiments, the treatment targets a biological pathway associated with a shared NP detected in the biological sample. A NP within a positive causal shared NP cluster is a positive causal NP, and a NP within a negative causal shared NP cluster is a negative causal NP. In certain embodiments, the method includes determining whether the patient has one or more symptoms of the second disease. In certain embodiments, the method includes selecting, recommending, and/or administering the treatment for the second disease to the patient, when the patient has one or more symptoms of the second disease, and the NPs detected/present in the biological sample comprises a higher number of positive causal NPs compared to negative causal NPs. In certain embodiments, the method includes recommending, performing with and/or administering one or more lifestyle changes for the second disease to the patient, when the patient does not have one or more symptoms of the second disease, and the NPs detected/present in the biological sample comprises a higher number of positive causal NPs compared to negative causal NPs. In certain embodiments, the first disease is lupus, and second disease is CAD. In certain embodiments, the second disease is CAD, and the treatment for CAD can a treatment for CAD mentioned below. In certain embodiments, the second disease is CAD, and the lifestyle change for CAD can a lifestyle change mentioned below.

An aspect of the present disclosure is directed to a method for determining a coronary artery disease (CAD) state of a patient. The method can include (i) detecting one or more SNPs selected from SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; in a biological sample from the patient; and determining a CAD state of the patient, based on the presence of the one or more SNPs in the biological sample. Determining a CAD state of the patient can include, determining whether the patient has CAD, the severity of the CAD, the type of CAD, and/or whether the patient is at risk of developing CAD. Determining that the patient is at risk of developing CAD can include determining the type of, and/or severity of CAD the patient is at risk of developing. In certain embodiments, determining a CAD state of the patient include, determining whether the patient has CAD, or whether the patient is at risk of developing CAD. The patient is determined to have CAD, or is at risk of developing CAD, when the one or more SNPs are present in the biological sample. The method can determine the severity of, type of CAD the patient has, or is at risk of developing, based on the SNPs present in the biological sample.

In certain embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6. In certain embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66. In some embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67. In certain embodiments, the one or more SNPs include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 SNPs. In certain embodiments, the one or more SNPs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451, or any value or range there between, SNPs. In certain embodiments, the one or more SNPs consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 or any value or range there between, SNPs. In certain embodiments, the one or more SNPs comprises 2 to 4,451 SNPs. In certain embodiments, the one or more SNPs comprises 2 to 10, 2 to 50, 2 to 100, 2 to 300, 2 to 100, 2 to 500, 2 to 1,000, 2 to 1,500, 2 to 2,000, 2 to 2,009, 2 to 4,451, 10 to 50, 10 to 100, 10 to 300, 10 to 100, 10 to 500, 10 to 1,000, 10 to 1,500, 10 to 2,000, 10 to 2,009, 10 to 4,451, 50 to 100, 50 to 300, 50 to 100, 50 to 500, 50 to 1,000, 50 to 1,500, 50 to 2,000, 50 to 2,009, 50 to 4,451, 100 to 300, 100 to 100, 100 to 500, 100 to 1,000, 100 to 1,500, 100 to 2,000, 100 to 2,009, 100 to 4,451, 300 to 100, 300 to 500, 300 to 1,000, 300 to 1,500, 300 to 2,000, 300 to 2,009, 300 to 4,451, 100 to 500, 100 to 1,000, 100 to 1,500, 100 to 2,000, 100 to 2,009, 100 to 4,451, 500 to 1,000, 500 to 1,500, 500 to 2,000, 500 to 2,009, 500 to 4,451, 1,000 to 1,500, 1,000 to 2,000, 1,000 to 2,009, 1,000 to 4,451, 1,500 to 2,000, 1,500 to 2,009, 1,500 to 4,451, 2,000 to 2,009, 2,000 to 4,451, or 2,009 to 4,451 SNPs. In certain embodiments, the one or more SNPs comprises at least 2, 10, 50, 100, 300, 100, 500, 1,000, 1,500, 2,000, or 2,009 SNPs. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs selected from each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein the number of SNPs selected from different Tables can be the same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs selected from each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein the number of SNPs selected from different Tables can be the same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of 1, 2, 3, 4, 5, 6, 7, 8, 9, 10, 11, 12, 13, 14, 15, 16, 17, 18, 19, 20, 21, 22, 23, 24, 25, or 26, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein number of SNPs selected from different Tables can be same or different. As an non-limiting illustrative example, the one or more SNPs include 3 SNPs from a respective Table can denote that the method include (i) detecting 3 SNPs from SNPs listed in the respective Table, in a biological sample from the patient; and determining the CAD state of the patient, based on the presence of the 3 SNPs in the biological sample. Detecting the one or more SNPs, in the biological sample can include detecting presence of the one or more SNPs in the biological sample. In certain embodiments, the patient is determined to have CAD, or is at risk of developing CAD, when the one or more SNPs detected (e.g., present in the biological sample) comprises one or more SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67. In certain embodiments, the patient is determined to have CAD, or is at risk of developing CAD, when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. In certain embodiments, the patient is determined to have CAD, or is at risk of developing CAD, when the one or more SNPs (e.g., detected/present in the biological sample) comprises higher number of risk SNPs compared to protective SNPs. In certain embodiments, the patient is determined to have CAD, or is at risk of developing CAD, when a high proportion of SNPs listed in the at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. SNPs within the positive causal clusters (e.g. SNPs within Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67) are risk SNPs, and SNPs within the negative causal clusters (e.g. SNPs within Tables: 13-10; 13-24; 13-25; 13-30; 13-40; 13-45; 13-50; 13-61; and 13-66) are protective SNPs. SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67, include all the SNPs listed in Tables 13-1 to 13-67. As a non-limiting illustrative example, “SNPs listed in Table X and Y” includes x+y SNPs, where Table X contains x SNPs and Table Y contains y SNPs, considering no overlap (e.g., the SNPS are different) exists between x and y SNPs, in the event of overlap, duplicate copies can be excluded from analysis.

The one or more SNPs may or may not include SNPs that are not listed in Tables 13-1 to 13-67. In certain embodiments, the one or more SNPs do not include any SNPs that are not listed in Tables 13-1 to 13-67. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67.

In certain embodiments, a disease risk score for the patient is calculated based on presence of the one or more SNPs in the biological sample, and the CAD state of the patient is determined based on the disease risk score.

Detecting the one or more SNPs, in the biological sample can include detecting whether the one or more SNPs are present in the biological sample. The one or more SNPs in the biological sample can be detected, e.g., whether the one or more SNPs are present in the biological sample can be detected, based on analyzing at least a portion of the nucleic acid of the patient in the biological sample. Presence of the one or more SNPs in the biological sample can be detected, based on analyzing at least a portion of the nucleic acid of the patient in the biological sample. The nucleic acid can be DNA and/or RNA. Analyzing at least a portion of the nucleic acid of the patient, can include analyzing at least a portion of RNA and/or at least a portion of DNA of the patient, in the biological sample. In certain embodiments, analyzing at least a portion of the nucleic acid includes analyzing the at least a portion of DNA of the patient, in the biological sample. In certain embodiments, analyzing at least a portion of the nucleic acid includes analyzing the at least a portion of RNA of the patient, in the biological sample. In certain embodiments, analyzing the RNA can include, analyzing mRNA. In certain embodiments, analyzing at least a portion of the nucleic acid includes RNA sequencing. In certain embodiments, analyzing at least a portion of the nucleic acid includes mRNA sequencing. In certain embodiments, analyzing at least a portion of the nucleic acid includes DNA sequencing. In certain embodiments, the method includes analyzing at least a portion of the nucleic acid of the patient in the biological sample. In certain embodiments, the method includes analyzing at least a portion of the nucleic acid of the patient in the biological sample to detect presence of the one or more SNPs in the biological sample from the patient. In certain embodiments, analyzing at least a portion of the nucleic acid includes measuring expression of the genes associated with the one or more SNPs. The genes associated with a SNPs, can include the E-, C-, T, and/or P-gene associated with the SNP. In Tables 13-1 to 13-67, genes associated with the SNPs listed in the Tables are listed. In certain embodiments, analyzing the nucleic acid includes performing enrichment analysis of the genes associated with the one or more SNPs. The enrichment analysis can be performed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment analysis is performed using GSVA.

In certain embodiments, the method includes analyzing at least a portion of the nucleic acid of the patient in the biological sample to detect presence of the one or more SNPs in the biological sample from the patient, and determining the CAD state of the patient based on the presence of the one or more SNPs in the biological sample, wherein the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, and the patient is determined to have CAD, or is determined to be at risk of developing CAD when i) the one or more SNPs (e.g., detected/present in the biological sample) comprises higher number of risk SNPs compared to protective SNPs.

The biological sample can be a blood sample, isolated peripheral blood mononuclear cells (PBMCs), tissue biopsy sample, nasal fluid, saliva, urine, stool, or any derivative thereof. In certain embodiments, the biological sample can be a blood sample or any derivative thereof. In certain embodiments, the biological sample can be PBMCs or any derivative thereof. In certain embodiment, the patient has lupus. In certain embodiments, the patient does not have lupus. In certain embodiments, the patient is at an elevated risk of having lupus. In certain embodiments, the patient is asymptomatic for lupus.

In certain embodiments, the method comprises determining one or more symptoms of CAD in the patient. The one or more symptoms of CAD can include symptoms as understood by one of ordinary skill in the art, or by a physician. Non-limiting symptoms of CAD symptoms can include symptoms identified from echocardiogram, exercise stress test, chest X-ray, cardiac catheterization, etc. In certain embodiments, the patient is determined to have CAD when the one or more SNPs are present in the biological sample. In certain embodiments, the patient is determined to have CAD when the one or more SNPs are present in the biological sample, and the patient has the one or more symptoms of CAD. In certain embodiments, the patient is determined to have CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both, and, ii) the patient has the one or more symptoms of CAD. In certain embodiments, the patient is determined to have CAD when i) the one or more SNPs (e.g., detected/present in the biological sample) comprises higher number of risk SNPs compared to protective SNPs and, ii) the patient has the one or more symptoms of CAD. In certain embodiments, the patient is determined to have CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. In certain embodiments, the patient is determined to be at risk of developing CAD when i) the one or more SNPs are present in the biological sample, and ii) one or more symptoms of CAD are absent in the patient. In certain embodiments, the patient is determined to be at risk of developing CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both, and ii) one or more symptoms of CAD are absent in the patient. In certain embodiments, the patient is determined to be at risk of developing CAD when i) the one or more SNPs (e.g., detected/present in the biological sample) comprises higher number of risk SNPs compared to protective SNPs and ii) one or more symptoms of CAD are absent in the patient. The method can determine the severity of, type of CAD the patient has, or is at risk of developing, based on the SNPs present in the biological sample.

In certain embodiments, the method comprises selecting, recommending, and/or administering a treatment to the patient, based on the CAD state of the patient. In certain embodiments, the treatment is selected, recommended, and/or administered based on the determination that the patient has CAD. In certain embodiments, the treatment is administered based on the determination that the patient has CAD. In certain embodiments, the treatment is selected, recommended, and/or administered based on the determination that the patient is at risk of developing CAD. In certain embodiments, the treatment is administered based on the determination that the patient is at risk of developing CAD. In certain embodiments, the method comprises administering the treatment to the patient, based on the CAD state of the patient. In certain embodiments, the treatment is selected, recommended, and/or administered based on i) the presence of the one or more SNPs in the biological sample from the patient, and/or ii) the patient having one or more symptoms of CAD. In certain embodiments, the treatment is administered based on i) the presence of the one or more SNPs in the biological sample from the patient, wherein the one or more SNPs (e.g., detected/present in the biological sample) comprises higher number of risk SNPs compared to protective SNPs, and/or ii) the patient having one or more symptoms of CAD, and the method can be directed to treating CAD. In certain embodiments, the treatment is administered based on i) the presence of the one or more SNPs in the biological sample from the patient, and/or ii) the patient having one or more symptoms of CAD, and the method can be directed to treating CAD. In certain embodiments, the treatment is administered based on i) the presence of the one or more SNPs in the biological sample from the patient, and ii) the patient having one or more symptoms of CAD, and the method can be directed to treating CAD. The treatment selected, recommended, and/or administered can be based on the one or more SNPs detected (e.g., present) in the biological sample. In certain embodiments, the treatment administered is based on the one or more SNPs detected (e.g., present) in the biological sample.

In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and/or ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and/or ii) the patient has one or more symptoms of CAD. In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and ii) the patient has one or more symptoms of CAD.

The treatment selected, recommended, and/or administered can be based on the SNPs present in the biological sample. The treatment administered can be based on the SNPs present in the biological sample. In certain embodiments, the treatment can be based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein one or more SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein one or more SNPs listed in the Table are present in the biological sample. In certain embodiments, the treatment is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein a high proportion of SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein a high proportion of SNPs listed in the Table are present in the biological sample. Treatments based on a functional annotation of a respective Table may target, i) one or more biological pathways (see for example, Table 13) associated with the respective Table, ii) one or more genes listed in the respective Table and/or iii) genes and/or biological pathways upstream of or related to the biological pathways associated with, gene listed in and/or SNPs listed in the respective Table. The treatment can include one or more treatments of CAD. In certain embodiments, the treatment is configured to treat CAD. In certain embodiments, the treatment is configured to reduce severity of CAD. In certain embodiments, the treatment is configured to reduce a risk of developing CAD. In certain embodiments, the treatment comprises a treatment for atherosclerosis. In certain embodiments, the treatment comprises an anti-IFN antibody such as anifrolumab; an anti-oxidized LDL antibody such as orticumab, an anti-PCSK9 such as alirocumab and/or evolocumab; a JAK inhibitor such as baricitinib and/or tofacitinib; a MTOR inhibitor rapamycin; a MPO inhibitor such as PF-1355; an ACE inhibitor such as captopril; a statin; or any combination thereof. In certain embodiments, the treatment comprises a pharmaceutical composition.

In certain embodiments, the patient is determined to be at risk of developing CAD, when the one or more SNPs detected are present in the biological sample, but the patient does not have one or more symptoms of CAD. In certain embodiments, the patient is determined to be at risk of developing CAD, when the one or more SNPs (e.g., detected/present in the biological sample) comprises higher number of risk SNPs compared to protective SNPs, but the patient does not have one or more symptoms of CAD. In certain embodiments, the patients is recommended, performed with and/or administered one or more lifestyle changes, when the one or more SNPs detected are present in the biological sample. In certain embodiments, the patients is recommended, performed with and/or administered one or more lifestyle changes, when the one or more SNPs detected are present in the biological sample, but the patient does not have one or more symptoms of CAD. In certain embodiments, the patients is recommended, performed with and/or administered one or more lifestyle changes, when the one or more SNPs (e.g., detected/present in the biological sample) comprises higher number of risk SNPs compared to protective SNPs. In certain embodiments, the patients is recommended, performed with and/or administered one or more lifestyle changes, when the one or more SNPs (e.g., detected/present in the biological sample) comprises higher number of risk SNPs compared to protective SNPs, but the patient does not have one or more symptoms of CAD. The one or more lifestyle changes can include monitoring, such as frequent monitoring the patient for one or more symptoms of CAD. Monitoring can include monitoring through echocardiogram, exercise stress test, chest X-ray, cardiac catheterization, etc, for one or more symptoms of CAD. Frequent monitoring can include a monitoring at a higher frequency, compared to past (e.g., past 1 month, 3 months, 6 months, 1 year, 2 years, 3 years, 5 years, 10 years, etc.) monitoring of the patient. Frequent monitoring can include a monitoring at a higher frequency, compared to the monitoring and/or recommended monitoring of a control subject having similar age, sex, ethnicity, and/or the like as of the patient. In certain embodiments, the one or more SNPs (e.g., detected/present in the biological sample) comprises higher number of risk SNPs compared to protective SNPs, and the method includes i) selecting, recommending, and/or administering the treatment to the patient when the patient has one or more symptoms of CAD, or ii) recommending, performing with and/or administering one or more lifestyle changes when the patient does not have one or more symptoms of CAD.

The patient can be a human patient.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

The current disclosure includes the following aspects.
1. A method for determining shared biological pathways, and shared nucleotide polymorphisms (NPs), between a first disease and a second disease, the method comprising:
   (a) selecting a first set of nucleotide polymorphisms (NPs) associated with the first disease from a first dataset, wherein the first dataset comprises data regarding association of a first plurality of NPs with the first disease;
   (b) mapping one or more NPs of the first set of NPs to genes, to identify a plurality of NP-mapped genes;
   (c) clustering the plurality of NP-mapped genes to obtain one or more gene clusters;
   (d) clustering the one or more NPs mapped in step (b) to obtain a first set of NP clusters;
   (e) performing a causal inference analysis to select a subset of NP clusters from the first set of NP clusters obtained in step (d), wherein each NP cluster within the subset of NP clusters has a positive or negative causal effect on the second disease; and
   (f) functionally annotating i) one or more NP clusters of the subset of NP clusters selected in step (e), and/or ii) gene clusters mapped with the one or more NP clusters of the subset of NP clusters, thereby determining the shared biological pathways between the first disease and the second disease, wherein the NPs within the NP clusters within the subset of NP clusters selected in step
   (e) are the shared NPs between the first and second disease.
2. The method of aspect 1, wherein the clustering of the one or more NPs mapped in step (b) to obtain the first set of NP clusters in step (d), is performed based on the clustering of the plurality of NP-mapped genes in step (c).
3. The method of aspect 1, wherein the p-value for statistical significance of the association of each NP in the first set of NPs with the first disease is lower than 1*10⁻⁶.
4. The method of aspect 1, wherein the p-value for statistical significance of the association of each NP in the first set of NPs with the first disease is lower than 5*10⁻⁸.
5. The method of any one of aspects 1 to 4, wherein in step (b) the plurality of NP-mapped genes are identified by mapping the one or more NPs of the first set of NPs independently to their i) associated expression quantitative trait loci (eQTL) expression genes (E-Genes), ii) associated transcription factors and downstream target genes (T-Genes), iii) associated protein coding genes (C-genes), and/or iv) proximal genes (P-genes).
6. The method of any one of aspects 1 to 5, wherein in step (c) the plurality of NP-mapped genes are clustered based on protein-protein interactions of proteins encoded by the NP-mapped genes, gene co-expression, genetic pathway, genetic annotations, genetic associations, or any combination thereof.
7. The method of aspect 6, wherein in step (c) the plurality of NP-mapped genes are clustered based on protein-protein interactions of the proteins encoded by the NP-mapped genes.
8. The method of aspect 7, wherein the clustering of the NP-mapped genes comprises:
   clustering the encoded proteins into one or more protein clusters, wherein proteins determined to be within same biological pathway are grouped into same protein cluster; and
   clustering the NP-mapped genes to form the one or more gene clusters of step (c), based at least on clustering of the encoded proteins, wherein for a respective protein cluster formed, a gene cluster is formed containing the genes that encodes the proteins within the respective protein cluster.
9. The method of any one of aspects 1 to 8, wherein the causal inference method of step (e) is a mendelian randomization (MR) based method.
10. The method of aspect 9, wherein the MR based method comprises determining a causal effect of the NP clusters of the first set of NP clusters on the second disease, wherein for a respective NP cluster, at least 2 NPs within the cluster collectively is used as instrument variables, summary statistics from a second dataset is used as exposure, and a third dataset is used as outcome, wherein the second dataset comprises the summary statistics regarding association of a second plurality of NPs with the first disease, and the third dataset comprises data regarding association of a third plurality of NPs with the second disease.
11. The method of aspect 10, wherein in the MR-based method, for a respective NP cluster of the first set of NP clusters the NPs used as instrument variables are selected based on the i) strength of association of the NPs with the first disease, ii) the strength of association of the NPs with the second disease, iii) the strength of association of NPs with confounding traits, iv) linkage disequilibrium with other NPs, v) genomic location, vi) allele harmonization between the second and third datasets, or any combination thereof.
12. The method of any one of aspects 1 to 11, wherein for a respective NP cluster of the subset of NP clusters selected in step (e), the functionally annotating comprises:
   (i) overlapping a gene cluster mapped with the respective NP cluster, with one or more gene function signature lists to determine, significant overlap between the gene cluster and the one or more gene function signature lists; and
   (ii) annotating the respective NP cluster with one or more functional characterizations, based at least on the significant overlap.
13. The method of any one of aspects 1 to 12, wherein the first disease is selected from lupus, coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder, inflammatory bowel disease, multiple sclerosis, and glomerulonephritis.
14. The method of any one of aspects 1 to 12, wherein the first disease is lupus.
15. The method of any one of aspects 1 to 13, wherein the second disease is different from the first disease and is selected from lupus, cardiovascular disease, CAD, myocardial infarction, ischemic stroke, coronary atherosclerosis, cardiomyopathy, depression, asthma, COPD, diabetes mellitus, nonalcoholic fatty liver disease, metabolic disorder, inflammatory bowel disease, multiple sclerosis, and glomerulonephritis.
16. The method of any one of aspects 1 to 14, wherein the second disease is CAD.
17. The method of any one of aspects 1 to 16, wherein the NPs are single nucleotide polymorphisms (SNPs), indels, or splice variants.
18. The method of any one of aspects 1 to 16, wherein the NPs are SNPs.
19. A method for determining shared biological pathways, and shared single nucleotide polymorphisms (SNPs) between lupus and a second disease, the method comprising:
   (a) selecting a first set of SNPs associated with lupus from a first dataset, wherein the first dataset comprises data regarding association of a first plurality of SNPs with lupus;
   (b) mapping one or more SNPs of the first set of SNPs selected in step (a), to genes to identify a plurality of SNP-mapped genes;
   (c) clustering the plurality of SNP-mapped genes based on protein-protein interaction of proteins encoded by the SNP-mapped genes to obtain one or more gene clusters;
   (d) clustering the one or more SNP mapped in step (b) to obtain a first set of SNP clusters, based on clustering of the plurality of SNP-mapped genes in step (c);
   (e) performing a Mendelian randomization (MR)-based method to select a subset of SNP clusters from the first set of SNP clusters obtained in step (d), wherein each SNP cluster within the subset of SNP clusters independently has a positive or negative causal effect on the second disease; and
   (f) functionally annotating i) one or more SNP clusters of the subset of SNP clusters selected in step (e), and/or ii) gene clusters mapped with the one or more SNP clusters of the subset of SNP clusters, thereby determining the shared biological pathways between lupus and the second disease,
wherein the SNPs in the SNP clusters within the subset of SNP clusters obtained in step (e) are the shared SNPs between lupus and the second disease.
20. The method of aspect 19, wherein the p-value for statistical significance of the association of the first set of SNPs with lupus is lower than about 1*10⁻⁴, lower than about 5*10⁻⁵, lower than about 1*10⁻⁵, lower than about 5*10⁻⁶, lower than about 1*10⁻⁶, lower than about 5*10⁻⁷, lower than about 1*10⁻⁷, lower than about 5*10⁻⁸, or lower than about 1*10⁻⁸.
21. The method of aspect 19, wherein the p-value for statistical significance of the association of each SNP in the first set of SNPs with lupus is lower than about 1*106.
22. The method of aspect 19, wherein the p-value for statistical significance of the association of each SNP in the first set of SNPs with lupus is lower than about 5*108.
23. The method of any one of aspects 19 to 22, wherein in step (b) the plurality of SNP-mapped genes are identified by mapping one or more SNPs of the subset of SNPs independently to their i) associated expression quantitative trait loci (eQTL) expression genes (E-.Genes), ii) associated transcription factors and downstream target genes (T-Genes), iii) associated protein coding genes (C-genes), and/or iv) proximal genes (P-genes).
24. The method of any one of aspects 19 to 23, wherein in step (c) the protein-protein interaction based clustering of the plurality of SNP-mapped genes comprises:
   clustering the encoded proteins into one or more protein clusters, wherein proteins determined to be within same biological pathway are grouped into same protein cluster; and
   clustering the SNP-mapped genes to form the one or more gene clusters of step (c), based at least on clustering of the encoded proteins, wherein for a respective protein cluster formed, a gene cluster is formed containing the genes that encodes the proteins within the respective protein cluster.
25. The method of any one of aspects 19 to 24, wherein the MR-based method in step (e) comprises determining a causal effect of the SNP clusters of the first set of SNP clusters on the second disease, wherein for a respective SNP cluster, at least 2 SNPs within the cluster, collectively is used as instrument variables, summary statistics from a second dataset is used as exposure, and a third dataset is used as outcome, wherein the second dataset comprises the summary statistics regarding association of a second plurality of SNPs with lupus, and the third dataset comprises data regarding association of a third plurality of SNPs with the second disease.
26. The method of aspect 25, wherein in the MR-based method of step (e) independently for each SNP cluster of the first set of SNP clusters SNPs used as instrument variables are selected based on the i) strength of association of the SNPs with lupus, ii) the strength of association of the SNPs with the second disease, iii) the strength of association of SNPs with confounding traits, iv) linkage disequilibrium with other SNPs, v) genomic location, vi) allele harmonization between the second and third datasets, or any combination thereof.
27. The method of any one of aspects 19 to 26, wherein for a respective SNP cluster of the subset of SNP clusters selected in step (e), the functionally annotating comprises:
   (i) overlapping a gene cluster mapped with the respective SNP cluster, with one or more gene function signature lists to determine, significant overlap between the gene cluster and the one or more gene function signature lists; and
   (ii) annotating the respective SNP cluster with one or more functional characterizations, based at least on the significant overlap.
28. The method of any one of aspects 19 to 27, wherein the second disease is selected from coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, and glomerulonephritis.
29. The method of any one of aspects 19 to 27, wherein the second disease is CAD.
30. A method for determining a coronary artery disease (CAD) state in a patient, the method comprising:
   detecting one or more SNPs selected from SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; in a biological sample from the patient; and
   determining the CAD state in the patient, based at least on the presence of the one or more SNPs in the biological sample.
31. The method of aspects 30, wherein the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6.
32. The method of aspect 30, wherein the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67.
33. The method of any one of aspect 30 to 32, wherein the one or more SNPs comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 SNPs.
34. The method of any one of aspects 30 to 33, wherein the one or more SNPs comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300, or all SNPs, selected from the SNPs listed in each of one or more Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein the number of SNPs selected from different Tables are same or different.
35. The method of any one of aspects 30 to 33, wherein the one or more SNPs comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300, or all SNPs, selected from the SNPs listed in each of one or more Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein the number of SNPs selected from different Tables are same or different.
36. The method of any one of aspects 30 to 35, wherein the presence of the one or more SNPs in the biological sample is detected by analyzing nucleic acid of the patient in the biological sample.
37. The method of any one of aspects 30 to 36, wherein the biological sample is a blood sample, isolated peripheral blood mononuclear cells (PBMCs), tissue biopsy sample, nasal fluid, saliva, urine, stool, or any derivative thereof.
38. The method of any one of aspects 30 to 37, a disease risk score for the patient is calculated based at least on the presence of the one or more SNPs in the biological sample, and the presence of the CAD state in the patient is determined based at least on the disease risk score.
39. The method of any one of aspects 30 to 38, wherein the patient has lupus.
40. The method of any one of aspects 30 to 38, wherein the patient is at an elevated risk of having lupus.
41. The method of any one of aspects 30 to 38, wherein the patient does not have lupus.
42. The method of any one of aspects 30 to 38, wherein the patient is asymptomatic for lupus.
43. The method of any one of aspects 30 to 38, further comprising administering a treatment to the patient based on the determined CAD state.
44. The method of aspect 43, wherein the treatment administered is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein one or more SNPs selected from the SNPs listed in the at least one Table, are present in the biological sample.
45. The method of aspect 43 or 44, wherein the treatment is configured to treat CAD.
46. The method of aspect 43 or 44, wherein the treatment is configured to reduce severity of CAD.
47. The method of aspect 43 or 44, wherein the treatment is configured to reduce a risk of developing CAD.
48. The method of aspect 43 to 47, wherein the treatment comprises a treatment for atherosclerosis.
49. The method of aspect 43 to 48, wherein the treatment comprises anti-IFN antibodies, anti-oxidized LDL antibodies, anti-PCSK9 antibodies, a JAK inhibitor, a MTOR inhibitor, a MPO inhibitor, an ACE inhibitor, statins, or any combination thereof.
50. The method of aspect 43 to 49, wherein the treatment comprises a pharmaceutical composition.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1D: Analysis of SNP-predicted genes associated with both SLE and CAD. FIG. 1A. Venn diagram of overlap between multi-ancestral SLE- and CAD-associated (p<10⁻⁶) SNPs. FIG. 1B. Venn diagram of overlap between SNP-predicted genes derived from regulatory elements (T-Genes), eQTL analysis (E-Genes), coding regions (C-Genes), and proximity within 5 kb (P-Genes). FIG. 1C. Application of S-LDSC using summary statistics for SLE, CVD, and CAD GWAS to estimate the heritability of the 189 SNP-predicted genes (top panel) and 135 SNP-predicted proteins (lower panel) from STRINGdb. Bar color indicates coefficient significance. FIG. 1D. PPI network consisting of 135 putative protein-coding genes. Functional and cell-type enrichments for each cluster were determined using BIG-C (black labels) and I-scope (red labels), respectively. Black labels over colored shadings represent shared BIG-C functional annotations for the clusters they surround.

FIGS. 2A-2D: MR demonstrates a positive association of effect sizes of SLE-associated non-HLA SNPs on SLE and CAD. FIG. 2A. Opposed plots of genetic association signals for SLE (Immunochip and GWAS overlayed) and CAD (meta-GWAS). Genomic position is shown on the x-axis, with alternating colors for each chromosome. Negative log₁₀ of the GWAS or Immunochip P-value for each SNP is shown on the y-axis. FIGS. 2B-2D. Forest plots of 6 MR causal estimates (beta±standard error). For results, light gray indicates insignificant (p>0.05), gray, positive causal, and dark gray, negative causal estimates determined by each MR method. Numbers on top of forest plots indicate the SNPs used as IVs after harmonization. FIG. 2B. Immunochip-derived SLE-associated non-HLA SNPs were used as IVs for SLE; summary statistics from both the SLE Immunochip study (left panel) and SLE GWAS (right panel) were used for the exposure; summary statistics from the CAD GWAS were used for the outcome. FIG. 2C. Additional MR analyses for validation. SNPs associated (p<10⁻⁶) with SLE in the Immunochip and GWAS study (row 1 and 2) or Phenoscanner platform reaching nominal significance (p<10⁻⁵, row 3) and reaching genome-wide significance (p<5×10⁻⁸, row 4) were used as IVs; summary statistics for the exposure and outcome are indicated. MR analyses with all SLE-associated SNPs (left column), excluding the entire short-arm of chromosome 6 (middle column), and excluding only the extended HLA region (chr6:27-34 Mb, right column). FIG. 2D. MR causal estimates of SLE on CAD using the same sets of SNPs in (FIG. 2C) for SLE-exposure as IVs, excluding pleiotropic SNPs associated (p<10⁻⁵) with either CAD-directly or CVD-related confounders, included on the Phenoscanner platform.

FIGS. 3A-3D: MR demonstrates a net positive-causal effect of SLE-associated non-HLA SNPs on CAD. FIG. 3A. MR diagram for testing the causal effects of SLE on CAD with respect to instrument relevance to the exposure, exclusion from the outcomes (i.e. CAD, MI, IS) and independence from confounding factors. LD-clumping (R²<0.001) was used to obtain independent IVs. FIG. 3B. Forest plots of MR causal estimates (beta ±standard error) for SLE on CAD (CAD-a, CAD-b), MI (MI-a, MI-b), IS, cardiomyopathy (CM) and atrial fibrillation (AFib) GWAS using 16 MR methods. Missing PRESSO-OC estimates indicate insignificant global tests for horizontal pleiotropy. For results, light gray indicates insignificant (p>0.05), gray, positive causal (p<0.05). FIG. 3C. Application of S-LDSC using summary statistics for SLE, CVD and CAD GWAS to estimate the heritability (coefficient ±standard error) of the 284 SNP-predicted genes (top panel) and 160 SNP-predicted proteins from STRINGdb (lower panel). Bar color indicates coefficient significance. FIG. 3D. Cluster metastructures for the 160 putative protein-coding genes are based on PPI networks, clustered using MCODE and visualized in Cytoscape. Node gradient shading is proportional to intra-cluster connectivity, cluster size indicates number of genes per cluster and edge weight indicates inter-cluster connections. Functional and cell-type enrichments for each cluster were determined using BIG-C (black labels) and I-scope (red labels), respectively. Black labels over colored shadings represent shared functional annotations for the clusters they surround.

FIGS. 4A-4D: Bidirectional MR summaries between SLE and CAD. Scatter plots showing GWAS effect size estimates on the exposure (x-axis) and outcome (y-axis) with each dot representing a SNP and lines representing MR-estimates (right label) of SLE on CAD, MI and IS (FIG. 4A-4B) and in the reverse direction, with CAD or MI as exposure and SLE as the outcome (FIG. 4C-4D). MR-IVW and MR-Egger heterogeneity test results (Q-value) indicate whether significant heterogeneity was detected (asterisks, p<0.05), which does not necessarily indicate biased causal estimates. MR-Egger intercept indicate whether significant (asterisks, p<0.05) directional horizontal pleiotropy was detected, which usually indicates biased causal estimates. N.s., not significant.

FIGS. 5A-5F: SLE-associated SNPs on chromosome 6 account for the majority of negative causal effects on CAD by SSMR. FIGS. 5A-5B. Forest plots (beta ±standard error) of the top 25 (by absolute value of causal estimates) positive (FIG. 5A) and negative (FIG. 5B) causal SNPs identified by SSMR using the Wald-ratio method. FIGS. 5C-5D. Pie charts illustrating the distribution of 119 positive (FIG. 5C) and 234 negative (FIG. 5D) causal SLE SNPs on CAD. FIGS. 5E-5F. Cluster metastructures for the 498 (FIG. 5E) 557 (FIG. 5F) predicted genes from positive and negative causal SNPs identified by single-SNP MR. Metastructures are based on PPI networks, clustered using MCODE and visualized in Cytoscape. Node gradient shading is proportional to intra-cluster connectivity, cluster size indicates number of genes per cluster and edge weight indicates inter-cluster connections. Functional and cell-type enrichments for each cluster were determined using BIG-C (bold labels) and I-scope (italics labels), respectively. Bold black labels over gray shaded regions represent shared functional annotations for the clusters they surround.

FIGS. 6A-6H: Analysis of SLE-associated SNP-predicted genes with causal effects on CAD by single-SNP MR. FIGS. 6A-6B. Forest plots (beta±standard error) of the top 25 (by absolute value of causal estimates) positive (FIG. 6A) and negative (FIG. 6B) causal non-HLA SNPs identified by single-SNP MR (SSMR) using the Wald ratio method. FIGS. 6C and 6E. Pie charts illustrating the chromosomal distribution of 80 positive (FIG. 6C) and 96 negative (FIG. 6E) causal SLE SNPs on CAD. FIGS. 6D and 6F. Cluster metastructures for the 200 (FIG. 6D) 184 (FIG. 6F) predicted genes from positive and negative causal SNPs identified by single-SNP MR. Node gradient shading is proportional to intra-cluster connectivity, cluster size indicates number of genes per cluster and edge weight indicates inter-cluster connections. Functional and cell-type enrichments for each cluster were determined using BIG-C (bold labels) and I-scope (italics labels), respectively. Bold black labels over shaded regions represent shared functional annotations for the clusters they surround. FIGS. 6G and 6H. S-LDSC using summary statistics for SLE, CVD and CAD GWAS to estimate the heritability (coefficient ±standard error) of genes (open bars) and SNP-predicted proteins (hashed bars) predicted by positive (FIG. 6G) and negative (FIG. 6H) causal SNPs determined by SSMR. Bar color indicates coefficient significance.

FIGS. 7A-7B: MR analyses for positive and negative causal SNPs determined by SSMR. Forest plots (beta ±standard error) of the 80 positive (FIG. 7A) and 96 negative (FIG. 7B) causal non-HLA SNPs identified by SSMR using the Wald ratio method, ordered by absolute value of causal estimates. The 80 non-HLA SNPs listed in FIG. 7A are, from top to bottom, rs7692514; rs11037296; rs75010564; rs78506915; rs73161005; rs706778; rs7810922; rs965355; rs12743484; rs34099611; rs11603249; rs11037278; rs11601828; rs12279248; rs10905718; rs10954650; rs1609265; rs13026988; rs10933559; rs73245892; rs1981601; rs2541117; rs6946131; rs4869313; rs2122775; rs2594113; rs7179733; rs4459332; rs209665; rs874610; rs6478522; rs11164848; rs11145763; rs116967545; rs10800816; rs12470957; rs6689858; rs1237290; rs9851386; rs1292043; rs144484752; rs16837131; rs13023380; rs2019097; rs62131887; rs6738825; rs425648; rs2022013; rs2456973; rs223881; rs56114296; rs1534154; rs10916668; rs3807134; rs13107612; rs13106926; rs34749007; rs35388091; rs4637409; rs34029191; rs17266594; rs1125271; rs17200824; rs13129744; rs10516487; rs12119966; rs34725611; rs11085727; rs2078087; rs12409487; rs1122259; rs4411998; rs11150610; rs34815476; rs11150608; rs729302; rs2061831; rs13426947; rs10168266; and rs10931481. The 96 non-HLA SNPs listed in FIG. 7B are, from top to bottom, rs6756736; rs12946196; rs8105429; rs34709472; rs4678000; rs7566072; rs10878246; rs4917385; rs4748857; rs928596; rs6449173; rs7442295; rs73066671; rs7821169; rs1052690; rs3785437; rs7540556; rs11057864; rs7915387; rs1053093; rs144705368; rs7927370; rs3766374; rs7121755; rs7899961; rs6945400; rs3733345; rs4293757; rs55816332; rs500600; rs79821347; rs12683801; rs7214635; rs9415635; rs55634455; rs10419198; rs138191786; rs9568353; rs9952980; rs79558495; rs1372372; rs16953685; rs72832915; rs2235947; rs35281701; rs4948496; rs7625006; rs7264711; rs55705316; rs6574349; rs118087357; rs2941509; rs6705304; rs28392589; rs12946188; rs113115305; rs113370572; rs12946792; rs12978179; rs112345383; rs600751; rs28621669; rs28723017; rs12947480; rs12938117; rs8079075; rs111469562; rs113233720; rs112880843; rs4252665; rs143123127; rs9907966; rs1453560; rs1131665; rs35865896; rs12272314; rs1055382; rs1061502; rs34889521; rs11246213; rs12805435; rs11246217; rs58688157; rs1124816; rs11539530; rs36060383; rs2396545; rs936469; rs4963128; rs12795572; rs12419694; rs10229001; rs10954213; rs10239340; rs4731536; and rs4731533.

FIGS. 8A-8C: Analysis of HLA SNP-predicted genes associated with both SLE and CAD. FIG. 8A. Forest plot showing GWAS effect sizes ±standard error for 30 HLA SNPs significantly (p<10⁻⁶) associated with both SLE and CAD. FIG. 8B. PPI network consisting of 69 putative protein-coding genes predicted from the 30 HLA SNPs. Functional and cell-type enrichments for each cluster were determined using BIG-C (black labels) and I-scope (red labels), respectively. Black labels over colored shadings represent shared functional annotations for the clusters they surround. FIG. 8C. Gene set enrichments for each cluster were determined using IPA and EnrichR. P-values are from Fisher's exact test that measures the significance of overlap between analysis-ready genes in each cluster and genes within an annotation, with red shading proportional to significance of each enrichment. The 30 HLA SNPs listed in FIG. 8A are, from top to bottom, rs389883; rs2523578; rs185819; rs2072633; rs2269426; rs2621321; rs2857101; rs241445; rs241446; rs241453; rs241440; rs2071472; rs2857106; rs2621322; rs241452; rs17034; rs241456; rs2859579; rs2071474; rs2071470; rs1894407; rs1894408; rs2856997; rs2856993; rs2071475; rs241451; rs2857103; rs2071473; rs2621323; and rs3130342.

FIGS. 9A-9D: SLE-derived gene network with causal implications on CAD and PPI-based MR. FIG. 9A. S-LDSC using summary statistics for SLE, CVD and CAD GWAS to estimate the heritability (coefficient ±standard error) of the 2,336 genes (open bars) and 1,501 proteins (hashed bars) predicted by 838 Immunochip SNPs associated with SLE. Bar color indicates coefficient significance. FIG. 9B. Functional and cell-type enrichments for cluster metastructures were determined using BIG-C (bold labels) and I-scope (italics labels), respectively. Bold black labels over colored shadings represent shared BIG-C functional annotations for the clusters they surround. Node size is proportional to the number of SNPs (height) mapping to the genes in each cluster (width). Tier 1 positive (TIP) and negative (TIN) clusters were significant for 14/16 MR methods used; tier 2 positive (T2P) and negative (T2N) were significant by MR-IVW or at least 7/16 MR-methods; gray unlabeled, insignificant. Thickness of the dotted border is roughly proportional to the negative log of the MR-IVW p-value. Solid black border indicates clusters with −log(MR-IVW p-value) >3. FIG. 9C. Forest plots from PPI-based MR showing estimates (beta±standard error) calculated by MR-IVW for select positive and negative clusters. FIG. 9D. PPI-based S-LDSC (coefficient ±standard error) using GWAS summary statistics for SLE, CVD and CAD to estimate the proportion of heritability captured by SNP-predicted genes corresponding to the indicated cluster. Bar color indicates coefficient significance.

FIGS. 10A-10B: Positive and negative causal estimates for PPI-based clusters using MR-IVW. PPI-based MR-IVW (beta ±standard error) using these the 46 (FIG. 10A) and 67 (FIG. 10B) clusters of SLE SNP-derived IVs in CAD, MI, IS, cardiomyopathy, and atrial fibrillation GWAS. For results, light gray indicates insignificant (p >0.05), dark gray with \hat{ } indicates positive causal at p<0.05; dark gray with * indicates negative causal at p<0.05 by IVW.

FIGS. 11A-11E: Comprehensive PPI-based MR predicts sets of SLE associated variants and pathways causal of CAD. FIG. 11A. S-LDSC using GWAS summary statistics for SLE, CVD and CAD to estimate the heritability (coefficient ±standard error) of the 3,272 genes (open bars) and 1,972 protein-coding genes (hashed bars) predicted by 1,708 combined Immunochip and Phenoscanner-derived SNPs. Bar color indicates coefficient significance. FIG. 11B. Functional and cell-type enrichments for cluster metastructures were determined using BIG-C (bold labels) and I-scope (italics labels), respectively. Bold black labels over gray shaded regions represent shared BIG-C functional annotations for the clusters they surround. Node size is proportional to the number of SNPs (height) mapping to the genes in each cluster (width). Tier 1 positive (TIP) and negative (TIN) clusters were significant for 14/16 MR methods used; tier 2 positive (T2P) and negative (T2N) (tier 1) were significant by MR-IVW or at least 7/16 MR-methods; gray unlabeled, insignificant. Clusters with mixed results are indicated (Mix). Thickness of the dotted border is roughly proportional to the negative log of the MR-IVW p-value. Solid black border indicates clusters with −log(MR-IVW p-value) >3. purple, mixed estimates; gray, insignificant. FIGS. 11C-11D. Forest plots from PPI-based MR showing estimates (beta ±standard error) calculated by 16 MR methods for select (FIG. 11C) positive and (FIG. 11D) negative clusters. Insignificant estimates are in light gray and denoted with a \hat{ }. The number of SNPs used as IVs for each cluster are indicated in the plots. FIG. 11E. PPI-based S-LDSC (coefficient ±standard error) using GWAS summary statistics for SLE, CVD and CAD to estimate the proportion of heritability captured by SNP-predicted genes corresponding to the indicated cluster. Bar color indicates coefficient significance.

FIGS. 12A-12D: Expected vs. observed MR-IVW casual estimates corresponding to random vs. PPI-based SNP-to-gene modules. FIG. 12A. Schematic illustrating the Monte Carlo Simulations for expected MR results using random sets of Immunochip-derived SNP-to-Gene modules. FIGS. 12B-12D Histograms representing the proportion of insignificant (p>0.05, light gray), positive causal (p<0.05, silver), negative causal (p<0.05, gray), positive causal (p<0.00075, dark gray), and negative causal (p<0.00075, black) results with respect to number of SNPs used as IVs for SLE-exposure on CAD corresponding to (FIG. 12B) the 46 SLE-derived clusters and (FIG. 12C) the comprehensive 67 SLE-derived clusters and (FIG. 12D) over 50,000 random sets of Immunochip-derived SNP-to-Gene modules. As indicated by the arrows in FIG. 12D, the histograms in each of FIGS. 12B-12D show when present, sequentially from bottom to top of each bar: positive causal p>0.05; positive causal p<0.00075; negative causal (p<0.05); negative causal (p<0.00075).

FIGS. 13A-13B: PPI-based MR identifies SLE SNPs with positive and negative causal effects on CAD. Forest plots (beta ±standard error) from 16 MR methods using summary statistics from the SLE GWAS (FIG. 13A) or SLE Immunochip (FIG. 13B) as the exposure and CAD GWAS as the outcome. SLE-associated non-HLA SNPs mapping to positive and negative clusters, separately (by tier) and together (“All SNPs”) were used as IVs after excluding CVD and confounder-associated SNPs followed by stringent LD-clumping (R²=0.001) and harmonization. The number of SNPs used as IVs for each SNP set are indicated in the plots. For results, light gray indicates insignificant (p >0.05); gray (p<0.05) positive; dark gray (p<0.05) negative by each MR method.

FIGS. 14A-14B: Genes and molecular pathways associated with positive causal clusters identify therapeutic interventions for managing CAD in SLE. FIG. 14A. All tier 1 and a selection of tier 2 clusters were functionally annotated using BIG-C, IPA and the EnrichR database. Select drugs acting on direct gene targets or on any of the associated pathways (italics) are listed. FIG. 14B. Venn diagram summarizing therapies that might uniquely impact SLE or CAD and those that may target pathways common to both diseases.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “Gini impurity” refers to a measure of how often a randomly chosen element from the set may be incorrectly labeled if it is randomly labeled according to the distribution of labels in the subset.

The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

One aspect of the present disclosure is directed to a method for determining shared biological pathways and/or shared nucleotide polymorphisms (NPs) between a first disease and a second disease. The method can also identify shared genes between the two diseases, and/or the biological pathways associated with the shared NPs and/or shared genes. The method can include any one of, any combination of, or all of steps (a)-(f). Step (a) can include selecting a first set of nucleotide polymorphisms (NPs) associated with the first disease from a first dataset. The first dataset can contain data regarding association of a first plurality of NPs with the first disease. Step (b) can include mapping one or more NPs of the first set of NPs selected in step (a) to genes, to identify a plurality of NP-mapped genes. Step (c) can include, clustering the plurality of NP-mapped genes to obtain one or more gene clusters. Step (d) can include, clustering the one or more NPs mapped in step (b), to obtain a first set of NP clusters. Step (e) can include performing a causal inference analysis to select a subset of NP clusters from the first set of NP clusters obtained in step (d). In certain embodiments, each NP cluster within the subset of NP clusters selected in step (e), has a positive or negative causal effect on the second disease, wherein the subset of NP clusters selected in step (e) includes NP clusters having positive causal effect on the second disease, and/or NP clusters having negative causal effect on the second disease. In certain embodiments, each NP cluster within the subset of NP clusters selected in step (e), has a positive causal effect on the second disease. In certain embodiments, each NP cluster within the subset of NP clusters selected in step (e), has a negative causal effect on the second disease. Step (f) can include functionally annotating i) one or more NP cluster of the subset of NP clusters selected in step (e), and/or ii) gene clusters mapped with the one or more NP cluster of the subset of NP clusters selected in step (e) thereby determining the shared biological pathways between the first and the second disease. The method can be performed in a computer.

The NPs within the NP clusters within the subset of NP clusters selected in step (e), are determined to be shared between the first disease and the second disease. For a respective NP within a NP cluster within the subset of NP clusters selected in step (e), the biological pathway associated with the respective NP, may be determined based on the functional annotation (e.g. as determined in step (f)) of the NP cluster, and/or of the gene cluster mapped to the NP cluster. Genes mapped with the NPs within the NP clusters within the subset of NP clusters selected in step (e), are determined to be shared between the first disease and the second disease. For a respective NP-mapped gene the associated biological pathway may be determined based on the functional annotation of the NP cluster (e.g., as determined in step (f)), within which the mapped NP of the respective NP-mapped gene is clustered into, and/or the functional annotation of the gene cluster within which the NP-mapped gene is clustered into. The shared biological pathways, NPs such as SNPs and genes between two diseases may represent biological processes, NPs such as SNPs and genes respectively involved in pathogenesis of both the diseases. As a non-limiting example, as described in Example 1, Table 8A, SNP cluster 2 was formed from Systemic lupus erythematosus (SLE) associated SNPs and have positive causal effect on CAD. The functional annotations of the cluster 2 (Table 8A cluster 2, and Table 13-2) include Glucocorticoid Receptor Signaling, Clathrin-mediated Endocytosis Signaling, Actin Nucleation by ARP-WASP Complex, Regulation of Actin-based Motility by Rho, Integrin Signaling, Neutrophil degranulation, Fc-gamma receptor signaling pathway involved in phagocytosis, Neutrophil Activation via Adherence on Endothelial Cells, Neutrophil Degranulation via FPR1/IL8, and Leukocyte Adhesion to Endothelial Cell. Therefore, the biological pathways shared between SLE and CAD, include Glucocorticoid Receptor Signaling, Clathrin-mediated Endocytosis Signaling, Actin Nucleation by ARP-WASP Complex, Regulation of Actin-based Motility by Rho, Integrin Signaling, Neutrophil degranulation, Fc-gamma receptor signaling pathway involved in phagocytosis, Neutrophil Activation via Adherence on Endothelial Cells, Neutrophil Degranulation via FPR1/IL8, and Leukocyte Adhesion to Endothelial Cell (including pathways obtained from functional annotation from other shared clusters). The SNPs within the cluster 2 (Table 13-2), and genes mapped to the SNPs within the cluster 2 (Table 13-2) are shared between SLE and CAD. The biological pathways associated with the SNPs within the cluster 2, and genes mapped to the SNPs within the cluster 2 (Table 13-2), are Glucocorticoid Receptor Signaling, Clathrin-mediated Endocytosis Signaling, Actin Nucleation by ARP-WASP Complex, Regulation of Actin-based Motility by Rho, Integrin Signaling, Neutrophil degranulation, Fc-gamma receptor signaling pathway involved in phagocytosis, Neutrophil Activation via Adherence on Endothelial Cells, Neutrophil Degranulation via FPR1/IL8, and Leukocyte Adhesion to Endothelial Cell (e.g., based on functional annotation of cluster 2). The shared biological pathways, SNPs and genes may represent biological processes, SNPs and genes respectively involved in pathogenesis of both SLE and CAD. The positive causal NPs may be risk NPs for both the first and second disease. The negative causal NPs may be risk NPs for the first disease, but protective NPs for the second disease.

In step (a), the first set of NPs can be selected from the first data set based at least on the p-value for statistical significance of the association of the NPs with the first disease. In certain embodiments, the p-value for statistical significance of the association of the first set of NPs with the first disease is lower than about 1*10⁻⁴, lower than about 5*10⁻⁵, lower than about 1*10⁻⁵, lower than about 5*10⁻⁶, lower than about 1*10⁻⁶, lower than about 5*10⁻⁷, lower than about 1*10⁻⁷, lower than about 5*10⁻⁸, or lower than about 1*10⁻⁸. In certain embodiments, the p-value for statistical significance of the association of each NP within the first set of NPs with the first disease is lower than about 1*10⁻⁶. In certain embodiments, the p-value for statistical significance of the association of each NP within the first set of NPs with the first disease is lower than about 5*10⁻⁸. In certain embodiments, non-HLA NPs from the first data set having the desired p-value (e.g., for statistical significance of the association with the first disease) are selected in step (a) to form the first set of NPs. The chromosomal non-HLA region may include all chromosomal regions excluding chromosomal HLA (chromosome 6 short arm), and/or chromosomal extended HLA region (chromosome 6:27-34 Mb).

In certain embodiments, in step (b) the plurality of NP-mapped genes are identified by mapping the one or more NPs of the first set of NPs to their i) associated expression quantitative trait loci (eQTL) expression genes (E-Genes), ii) associated transcription factors and downstream target genes (T-Genes), iii) associated protein coding genes (C-genes), iv) proximal genes (P-genes), or any combination thereof. The one or more NPs of the first set of NPs can be mapped to their associated E-genes, T-genes, C-genes, and/or P-genes using a suitable method, as understood by a person of ordinary skill in the art. In certain embodiments, all the NPs of the first set of NPs are mapped to genes to identify the plurality of NP-mapped genes. In certain embodiments, the NPs are single nucleotide polymorphism (SNPs), and non limiting methods for mapping SNPs to their associated E-genes, T-genes, C-genes, and/or P-genes can include the mapping method described in Owen et al., Analysis of trans-ancestral SLE risk loci identifies unique biologic networks and drug targets in African and European Ancestries. The American Journal of Human Genetics, 2020 107(5), 864-881; Fulco et al., Activity-by-Contact model of enhancer-promoter regulation from thousands of CRISPR perturbations. Nat Genet. 2019 51(12), 1664-1669; Nasser et al., Genome-wide enhancer maps link risk variants to disease genes. Nature 2021 593(7858), 238-243; or the like, all of which are incorporated herein by reference in its entirety. In certain embodiments, the SNPs are mapped to the associated E-genes, T-genes, C-genes, and/or P-genes according to the mapping method described in Owen et al., Analysis of trans-ancestral SLE risk loci identifies unique biologic networks and drug targets in African and European Ancestries. The American Journal of Human Genetics, 2020 107(5), 864-881. In a non-limiting example, expression quantitative trait loci (eQTLs) were identified using GTEx and the Blood eQTL browser database and mapped to their associated eQTL expression genes (E-Genes); to find SNPs in enhancers and promoters, in intergenic regions, and their associated transcription factors and downstream target genes (T-Genes), the atlas of Human Active Enhancers to interpret Regulatory variants (HACER) and the GeneHancer database, were queried; to find structural SNPs in protein-coding genes (C-Genes), the human Ensembl genome browser (GRCh38.p12) and dbSNP, were queried; and the other SNPs were linked to the most proximal gene (P-Gene) or gene region within about 4 to 6 kb, such as about 5 kb using the Ensembl Variant Effect Predicter (VEP). It will be evident to a skilled artisan, that associated E-genes, T-genes, C-genes, and/or P-genes for NPs can be identified using any suitable databases.

In step (c) the plurality of NP-mapped genes are clustered, wherein genes (e.g. NP-mapped genes) determined to be associated with same network of genes and/or within same biological pathway are grouped in the same gene cluster. Gene clustering can be performed based on any suitable method, such as protein-protein interactions of proteins encoded by the NP-mapped genes, gene co-expression, genetic pathway, genetic annotations, genetic associations, or any combination thereof; and can be performed using any suitable database. In certain embodiments, the plurality of NP-mapped genes are clustered based on protein-protein interactions of the proteins encoded by the NP-mapped genes. In certain embodiments, protein coding NP-mapped genes are clustered based on protein-protein interactions of the proteins encoded by the protein coding NP-mapped genes. In certain embodiments, non protein coding genes may not be clustered in step (c), and non protein coding genes and NP mapped with the non protein coding genes may be excluded from the method. In certain embodiments, the protein-protein interactions based clustering of the NP-mapped genes includes i) clustering the encoded proteins (e.g. by the NP-mapped genes) into one or more protein clusters, and ii) clustering the NP-mapped genes to form the one or more gene clusters of step (c), based on clustering of the encoded proteins, wherein for a respective protein cluster formed in step (c)-(i), in step (c)-(ii) a gene cluster is formed containing the genes that encodes the proteins within the respective protein cluster. As a non-limiting illustrative example, if gene A encodes protein 1, gene B encodes protein 2, gene C encodes protein 3, and gene D encodes protein 4, and protein 1 and 2 are clustered in one protein cluster, and protein 3 and 4 are clustered in another protein cluster, then genes A and B are clustered into one gene cluster and gene C and D are clustered into another gene cluster. Clustering of the encoded proteins, into the one or more protein clusters, e.g., as in (i) of step (c), can include grouping proteins determined to be within same biological pathway within the same protein cluster. The encoded proteins can be clustered into the one or more protein clusters based on physical interaction and/or functional association among the encoded proteins, wherein for a respective protein cluster, each protein within the cluster is determined to be capable of physically interacting and/or functionally associated, with at least one other protein within the cluster. Without intending to be limited by theory, it is believed that, physically interacting and/or functionally associated proteins may belong to the same biological pathway. The encoded proteins can be clustered into the one or more protein clusters using any suitable database, including but not limited to STRING, GIANT, Reactome, GeneMANIA, ReactomeFI, InBioMap, ConsensusPATHDB, HumanNet, BIND, PathwatCommons, HPRD, IRefindex, PID, BioGRID, HINT, DIP, Mentha, MultiNet, BioPlex, IntAct, and HumanInteratome. In certain embodiments, the clustering of the encoded proteins is performed using STRING database. In certain embodiments, the protein clusters were generated using STRING database, and were visualized using Cytoscape, MCODE plugin. It is evident to a skilled artisan, that the gene clustering and/or protein clustering of step (c) can be performed using any suitable database that is configured to cluster genes and/or proteins based on their associated biological pathways.

The one or more NPs mapped in step (b), can be clustered in step (d) using a suitable method as understood by a person of ordinary skill in the art, where NPs determined to be associated with same network of NPs and/or within same biological pathway are grouped in the NP same cluster. In certain embodiments, in step (d), the one or more NPs mapped in step (b) are clustered based on the clustering of the plurality of NP-mapped genes in step (c), to obtain the first set of NP clusters. In certain embodiments, in step (b) NPs of the first set of NPs are mapped to genes to identify the plurality of NP-mapped genes, and the NPs of the first set of NPs are clustered based on the clustering of the plurality of NP-mapped genes in step (c), to obtain the first set of NP clusters of step (d). The first set of NP clusters can contain one or more NP clusters. In some embodiments, for, a gene cluster formed in step (c), in step (d) a NP cluster containing the mapped NPs to the genes of the gene cluster, is formed. As a non-limiting illustrative example, if in step (b) NP 1 is mapped to gene A, NP 2 is mapped to gene B, NP 3 is mapped to gene C, NP 4 is mapped to gene D, and NP 5 is mapped to gene B; and if in step (c) genes A and B are grouped together in one gene cluster, and genes C and D are grouped together in another gene cluster; then in step (d) NPs 1, 2 and 5 are grouped into one NP cluster, and NPs 3 and 4 are grouped into another NP cluster of the first set of NP clusters. In certain embodiments, the one or more NPs mapped in step (b), can be clustered in step (d) can be clustered based on association between the NPs

The causal inference analysis of step (e) can include a causal inference method, such as Mendelian randomization based method. The causal inference method, such as the Mendelian randomization based method of step (e) can include, determining causal effect of the NP clusters of the first set of NP clusters obtained in step (d), on the second disease, wherein the NP clusters having positive causal effect, and/or NP clusters having negative on the second disease are selected to form the subset of NP clusters of step (e). In certain embodiments, causal inference method, such as the Mendelian randomization based method of step (e) can include, determining causal effect of the NP clusters of the first set of NP clusters obtained in step (d), on the second disease, wherein the NP clusters having positive causal effect on the second disease, and NP clusters having negative causal effect on the second disease, are selected to form the subset of NP clusters of step (e). In certain embodiments, causal inference method, such as the Mendelian randomization based method of step (e) can include, determining causal effect of the NP clusters of the first set of NP clusters obtained in step (d), on the second disease, wherein the NP clusters having positive causal effect on the second disease are selected to form the subset of NP clusters of step (e). In certain embodiments, causal inference method, such as the Mendelian randomization based method of step (e) can include, determining causal effect of the NP clusters of the first set of NP clusters obtained in step (d), on the second disease, wherein the NP clusters having negative causal effect on the second disease are selected to form the subset of NP clusters of step (e). For causal analysis of a respective NP-cluster of the first set of NP clusters of step (d), at least 2 NPs within the respective NP cluster are used as instrument variables (IVs). For causal analysis of a respective NP-cluster of the first set of NP clusters of step (d), at least 2 NPs within the respective NP cluster collectively are used as instrument variables, summary statistics from a second dataset is used as exposure, and a third dataset is used as outcome. The second dataset can include data regarding summary statistics of association of a second plurality of NPs with the first disease. The second data set can be same or different than the first dataset. In certain embodiments, the first data set and the second data set are same. In certain embodiments, the first data set and the second data set are different, and the first plurality of NPs overlap at least partially overlap with the second plurality of NPs, e.g., at least a portion of the NPs listed within the first dataset are also listed in the second dataset. The third dataset can include data regarding association of a third plurality of NPs with the second disease. In certain embodiments, the third dataset contains data regarding summary statistics of association of the third plurality of NPs with the second disease. In certain embodiments, the third plurality of NPs overlap at least partially with the first plurality of NPs, e.g., at least a portion of the NPs listed within the first dataset are also listed in the third dataset. In certain embodiments, the third plurality of NPs overlap at least partially with the second plurality of NPs, e.g., at least a portion of the NPs listed within the second dataset are also listed in the third dataset. In certain embodiments, the overlap between the first and second plurality of NPs at least partially overlap with the third plurality of NPs, e.g., at least a portion of the NPs that are listed within both the first dataset and the second dataset are also listed in the third dataset. In certain embodiments, proxy NPs are used in place of one or more NPs from the first plurality of NPs. Proxy NPs can be NPs that are in high linkage disequilibrium, e.g. are highly correlated, with one or more NPs from the first plurality of NPs.

In certain embodiments, for causal analysis of a respective NP cluster within the first set of NP clusters obtained in step (d), at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP cluster, collectively are used as the instrument variable. In certain embodiments, for causal analysis of each NP cluster within the first set of NP clusters, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP cluster, collectively are used as the instrument variable, wherein for different NP clusters the number of NPs used can be same or different.

For a NP cluster of the first set of NP clusters, the NPs used as IVs for the causal inference analysis of step (e), can be selected based on the i) strength of association of the NPs with the first disease, ii) the strength of association of the NPs with the second disease, iii) the strength of association of NPs with confounding traits, iv) linkage disequilibrium with other NPs, v) genomic location, vi) allele harmonization between the second and third datasets, or any combination thereof. For each NP cluster of the first set of NP clusters, the NPs used as IVs for the causal inference analysis of step (e), can be selected based independently on the i) strength of association of the NPs with the first disease, ii) the strength of association of the NPs with the second disease, iii) the strength of association of NPs with confounding traits, iv) linkage disequilibrium with other NPs, v) genomic location, vi) allele harmonization between the second and third datasets, or any combination thereof. In certain embodiments, the strength of association with the first disease of the NPs used as IVs has a threshold nominal or genome-wide significance, depending on the genotyping method and/or sample size of genetic association study. In certain embodiments, the strength of association with the first disease of the NPs used as IVs has i) a nominal significance p value <1*10\hat{ }4, <5*10\hat{ }4, <1*10\hat{ }5, <5*10\hat{ }5, <1*10\hat{ }6, <5*10\hat{ }6, <1*10\hat{ }7, <5*10\hat{ }7, <11*10\hat{ }7, or <5*10\hat{ }8, and/or ii) a genome-wide significance p-value <5*10\hat{ }6, <1*10\hat{ }7, <5*10\hat{ }7, <1*10\hat{ }7, <5*10\hat{ }8, <1*10\hat{ }8, <5*10\hat{ }9, or <1*10\hat{ }9. In certain embodiments, the strength of association with the first disease of the NPs used as IVs has i) a nominal significance p value <1*10\hat{ }5. In certain embodiments, the strength of association with the first disease of the NPs used as IVs has i) a nominal significance p value <1*10\hat{ }-6. In certain embodiments, the strength of association with the first disease of the NPs used as IVs has a genome-wide significance p-value <₅*10\hat{ }-8. In certain embodiments, the strength of association with the first disease of NPs used as IVs has p-value <1*10\hat{ }5 or more significant than genome-wide significance (p-value<5*10\hat{ }8). In certain embodiments, p-value for the strength of association of NPs selected as IVs, with the first disease is <1*10\hat{ }5. In certain embodiments, p-value for the strength of association of NPs selected as IVs, with the first disease is <1*10\hat{ }-6. In certain embodiments, NPs associated (e.g., p-value <1*10\hat{ }-4, <5*10\hat{ }4, <1*10\hat{ }5, <5*10\hat{ }5, <1*10\hat{ }6, <5*10\hat{ }6, <1*10\hat{ }7, <5*10\hat{ }7, <1*10\hat{ }7, or <5*10\hat{ }-8) with the second disease are excluded from using as IVs. In certain embodiments, for the NPs selected as IVs i) p-value for the strength of association with the first disease is <1*10\hat{ }5, and ii) p-value for the strength of association with the second disease is >1*10\hat{ }5. In certain embodiments, for the NPs selected as IVs i) p-value for the strength of association with the first disease is <1*10\hat{ }5, ii) p-value for the strength of association with the second disease is >1*10\hat{ }5, and/or iii) p-value for the strength of association with confounding traits is >1*10\hat{ }5. In certain embodiments, for causal analysis of a respective NP-cluster of the first set of NP clusters of step (d), the NPs of the respective NP cluster used as IVs have i) p-value for the strength of association with the first disease <1*10\hat{ }5, and ii) p-value for the strength of association with the second disease >1*10\hat{ }-5. In certain embodiments, for causal analysis of a respective NP-cluster of the first set of NP clusters of step (d), NPs of the respective NP cluster used as IVs have i) p-value for the strength of association with the first disease <1*10\hat{ }5, ii) p-value for the strength of association with the second disease >1*10\hat{ }5, and/or iii) p-value for the strength of association with the confounding traits >₁*10\hat{ }-5. In certain embodiments, NPs associated with the second disease, with p-value <1*10\hat{ }5, are excluded from using as IVs. In certain embodiments, NPs associated (e.g., p-value <1*10\hat{ }4, <5*10\hat{ }4, <1*10\hat{ }5, <5*10\hat{ }5, <1*10\hat{ }6, <5*10\hat{ }6, <1*10\hat{ }7, <5*10\hat{ }7, <1*10\hat{ }7, or <5*10\hat{ }-8) with confounding traits are excluded from using as IVs. In certain embodiments, NPs associated with confounding traits, with p-value <1*10\hat{ }5, are excluded from using as IVs. NPs associated with confounding traits can include NPs with known pleiotropic associations (such as p<1*10⁻⁵) to the second disease, and/or NPs associated with known risk factors of the second disease. In certain embodiments, the significance of association with the second disease and/or potential confounders of NPs to be excluded from IVs can be low (e.g. p-value <1*10\hat{ }5) or reach genome-wide (p-value <5*10\hat{ }-8) significance. In certain embodiments, the significance of association with the second disease and/or potential confounders of NPs to be excluded from IVs can be low (e.g. p-value <1*10\hat{ }5) or reach genome-wide (p-value <5*10\hat{ }8) significance. The NPs used as IVs can be independent of each other. In certain embodiments, the level of correlation, or linkage disequilibrium, between the NPs used as IVs has r\hat{ }2<0.0001, <0.0005, <0.001, <0.005, <0.01, <0.05, <0.1, or <0.5. In certain embodiments, the level of correlation, or linkage disequilibrium, between the NPs used as IVs has r\hat{ }2<0.001. In certain embodiments, the level of correlation, or linkage disequilibrium, between the NPs used as IVs has r\hat{ }2<0.01. In certain embodiments, the level of correlation, or linkage disequilibrium, between the NPs used as IVs has r\hat{ }2<0.1. In certain embodiments, the level of correlation, or linkage disequilibrium, between the NPs used as IVs has r\hat{ }2<0.5. In certain embodiments, an independent set of NPs is obtained using the clump_data( ) function in the TwoSampleMR R package, and is used as IVs. In certain embodiments, NPs in genomic regions, such as the major histocompatibility complex (MHC) or HLA region on the short-arm of chromosome 6, that are difficult to genotype, have extensive linkage disequilibrium or pleiotropy, and/or are unreliable, are excluded from using as IVs. In certain embodiments, NPs from MHC or HLA region on the short-arm of chromosome 6 are excluded from using as IVs. In certain embodiments, allele harmonization between the second and third dataset is performed to ensure the summary statistics for the first and second disease are based on the same reference and alternative alleles for each NP used as IVs. In certain embodiments, allele harmonization can be performed using the harmonise_data( ) function in the TwoSampleMR R package. In certain embodiments, NPs used as IVs are selected based on type of NP (e.g. coding, expression, transcription factor, proximal, etc.). In certain embodiments, coding NPs are used as IVs. In certain embodiments, NPs used as IVs are selected based on type of genomic region the NP occurs in (e.g. coding gene, non-coding gene, exon, intron, untranslated regions, eQTLs, transcription factor motifs, promoters, enhances, or other regulatory elements, etc.). In certain embodiments, NPs occurring in coding genes are used as IVs. In certain embodiments, NPs that are mapped to multiple genes and/or assigned to multiple clusters are excluded from being IVs for specific or all clusters. NPs selection of NPs for use as IVs can depend on the type of causal inference, such MR method being used for performing the causal inference analysis of step (e). In certain embodiments, in step (e) stringent set of NP cluster-specific instrumental variables can be used, and NPs with i) weak (e.g., p-value >5*10⁻⁸) associations with the first disease; ii) association or confounding effect (e.g., p-value <1*10⁻⁵) on the second disease; iii) weak mapping to gene(s) (e.g. only by proximity or eQTL in irrelevant cell types); or any combination thereof, are removed, prior to the causal inference, such as Mendelian randomization based method. In certain embodiments, such NPs (e.g., mentioned in the previous line) are not removed, during the causal inference analysis of step (e). The NPs having confounding effect on the second disease, can include NPs with known pleiotropic associations (such as p<1*10⁻⁵) to the second disease, and/or NPs associated with known risk factors of the second disease. NPs selected as IVs can have any one of, any combination of or all, of the properties mentioned in the herein, such as in this paragraph.

In certain embodiments, the selection of the NP cluster in step (e) can be based on the p-value of the causal effect. In certain embodiments, i) for the positive causal NP-clusters (e.g., clusters having positive causal effect on the second disease) selected in the step (e) the p-value for the positive causal estimate on the second disease is below 0.1, below 0.08, below 0.06, below 0.05, below 0.01, below 0.005, below 0.001, below 0.0005, or below 0.0001, and/or ii) for the negative causal NP-clusters (e.g., clusters having negative causal effect on the second disease) selected in the step (e) the p-value for the negative causal estimate on the second disease is below 0.1, below 0.08, below 0.06, below 0.05, below 0.01, below 0.005, below 0.001, below 0.0005, or below 0.0001. In certain embodiments, i) for the positive causal NP-clusters selected in the step (e) the p-value for the positive causal estimate on the second disease is below 0.05, and/or ii) for the negative causal NP-clusters selected in the step (e) the p-value for the negative causal estimate on the second disease is below 0.05. In certain embodiments, i) for each positive causal NP-clusters selected in the step (e) the p-value for the positive causal estimate on the second disease is below 0.05, and ii) for each negative causal NP-clusters selected in the step (e) the p-value for the negative causal estimate on the second disease is below 0.05. In certain embodiments, the NP-clusters selected in the step (e), has positive causal effect on the second disease. In certain embodiments, the NP-clusters selected in the step (e), has negative causal effect on the second disease. In certain embodiments, the NP-clusters selected in the step (e), has positive causal effect on the second disease, and the p-value for the positive causal estimate of each NP cluster of the subset of NP clusters selected in step (e), on the second disease is below 0.05. In certain embodiments, i) the p-value for the positive causal estimate of each positive causal NP cluster selected in step (e), on the second disease is below 0.05 and ii) the p-value for the negative causal estimate of each negative causal NP cluster selected in step (e), on the second disease is below 0.05. In certain embodiments, the NP-clusters selected in the step (e), has negative causal effect on the second disease, and the p-value for the negative causal estimate of each NP cluster of the subset of NP clusters selected in step (e), on the second disease is below 0.05.

In certain embodiments, the selection of the NP cluster in step (e) can be based on Bonferroni-corrected p-value of the causal effect. In certain embodiments, i) for the positive causal NP-clusters selected in the step (e), the Bonferroni-corrected p-value threshold for the positive causal estimate on the second disease is 0.1/[Total number of NP clusters selected], 0.08/[Total number of NP clusters selected], 0.06/[Total number of NP clusters selected], 0.05/[Total number of NP clusters selected], 0.01/[Total number of NP clusters selected], 0.0054[Total number of NP clusters selected], 0.001/[Total number of NP clusters selected], 0.00054[Total number of NP clusters selected], or 0.0001/[Total number of NP clusters selected], and/or ii) for the negative causal NP-clusters selected in the step (e), the Bonferroni-corrected p-value threshold for the negative causal estimate on the second disease is 0.1/[Total number of NP clusters selected], 0.08/[Total number of NP clusters selected], 0.06/[Total number of NP clusters selected], 0.054[Total number of NP clusters selected], 0.01/[Total number of NP clusters selected], 0.0054[Total number of NP clusters selected], 0.001/[Total number of NP clusters selected], 0.00054[Total number of NP clusters selected], or 0.0001/[Total number of NP clusters selected]. In certain embodiments, i) for the positive causal NP-clusters selected in the step (e), the Bonferroni-corrected p-value threshold for the positive causal estimate on the second disease is 0.054[Total number of NP clusters selected], and/or ii) for the negative causal NP-clusters selected in the step (e), the Bonferroni-corrected p-value threshold for the negative causal estimate on the second disease is 0.054[Total number of NP clusters selected]. In certain embodiments, i) for each positive causal NP-clusters selected in the step (e), the Bonferroni-corrected p-value threshold for the positive causal estimate on the second disease is 0.054[Total number of NP clusters selected], and ii) for each negative causal NP-clusters selected in the step (e), the Bonferroni-corrected p-value threshold for the negative causal estimate on the second disease is 0.054[Total number of NP clusters selected]. In certain embodiments, i) for each positive causal NP-clusters selected in the step (e), the Bonferroni-corrected p-value threshold for the positive causal estimate on the second disease is 0.0075, and ii) for each negative causal NP-clusters selected in the step (e), the Bonferroni-corrected p-value threshold for the negative causal estimate on the second disease is 0.0075.

In certain embodiments, the Bonferroni-corrected p-value threshold for the positive causal estimate on the second disease of the NP-clusters selected in the step (e) is 0.05/[Number of NP clusters selected]. In certain embodiments, the Bonferroni-corrected p-value threshold for the negative causal estimate on the second disease of the NP-clusters selected in the step (e) is 0.05/[Number of NP clusters selected]. Causal inference analysis using MR based method is described in Gupta et al., Mendelian randomization’: an approach for exploring causal relations in epidemiology. Public Health, 2017 145, 113-119, which is incorporated herein by reference in its entirety. The causal inference analysis of step (e) can be performed using one or more suitable causal inference, such as MR based methods. In certain embodiments, the causal inference analysis of step (e) can be performed using a plurality of causal inference, such as MR based methods, and each NP-cluster selected in the step (e), has positive causal effect on the second disease based on at least two causal inference methods, or negative causal effect on the second disease based on at least two causal inference methods. In certain embodiments, the causal inference analysis of step (e) can be performed using a plurality of MR based methods, and each NP-cluster selected in the step (e), has positive causal effect on the second disease based on at least two MR based methods, or negative causal effect on the second disease based on at least two MR based methods. Non-limiting examples of the MR based methods used in step (e) can include inverse-weighted (IVW), IVW-random effects, IVW-fixed effects, simple mode, simple mode-NOME, weighted mode, weighted mode-NOME, simple median, weighted median, penalized weighted median, two sample maximum likelihood, Maximum likehoods, RAPS, Egger, Egger-bootstrap, PRESSO-raw, PRESSO-OC, or any combination thereof. In certain embodiments, the causal inference analysis of step (e), is performed using a plurality of MR based methods, and each NP-cluster selected in the step (e), has positive causal effect on the second disease based on at least 40%, at least 43.75 00 at least 50%, at least 60%, at least 70 00 at least 80%, or at least 85%, or at least 87.5 00 of the MR based methods used, or negative causal effect on the second disease based on at least 40%, at least 43.75%, at least 50%, at least 60%, at least 70%, at least 80%, or at least 85%, at least 87.5%, of the MR based methods used. In certain embodiments, the causal inference analysis of step (e), is performed using a plurality of MR based methods, and each NP-cluster selected in the step (e), has positive causal effect on the second disease based on at least 87.5% of the MR based methods used (such as based on at least 14 out of 16 methods), or negative causal effect on the second disease based on at least 87.5%, of the MR based methods used. In certain embodiments, the causal inference analysis of step (e), is performed using a plurality of MR based methods, and each NP-cluster selected in the step (e), has positive causal effect on the second disease based on at least 43.75% of the MR based methods used (such as based on at least 7 out of 16 methods), or negative causal effect on the second disease based on at least 43.75%, of the MR based methods used. In certain embodiments, the causal inference analysis of step (e), is performed using a plurality of MR based methods wherein the plurality of MR based methods includes IVW, and each NP-cluster selected in the step (e), has positive causal effect on the second disease based at least on IVW, or negative causal effect on the second disease based at least on IVW. In certain embodiments, the causal inference analysis of step (e), is performed using a plurality of MR based methods, and each NP-cluster selected in the step (e), has positive causal effect on the second disease based on at least 40%, at least 43.75%, at least 50%, at least 60%, at least 70%, at least 80%, or at least 85%, or at least 87.5%, of the MR based methods used. In certain embodiments, the causal inference analysis of step (e), is performed using a plurality of MR based methods, and each NP-cluster selected in the step (e), has positive causal effect on the second disease based on at least 87.5% of the MR based methods used. In certain embodiments, the causal inference analysis of step (e), is performed using a plurality of MR based methods, and each NP-cluster selected in the step (e), has positive causal effect on the second disease based on at least 43.75% of the MR based methods used. In certain embodiments, the causal inference analysis of step (e), is performed using a plurality of MR based methods wherein the plurality of MR based methods includes IVW, and each NP-cluster selected in the step (e), has positive causal effect on the second disease based at least on IVW. In certain embodiments, the causal inference analysis of step (e), is performed using a plurality of MR based methods, and each NP-cluster selected in the step (e), has negative causal effect on the second disease based on at least 40%, at least 43.75%, at least 50%, at least 60%, at least 70%, at least 80%, or at least 85%, or at least 87.5%, of the MR based methods used. In certain embodiments, the causal inference analysis of step (e), is performed using a plurality of MR based methods, and each NP-cluster selected in the step (e), has negative causal effect on the second disease based on at least 87.5% of the MR based methods used. In certain embodiments, the causal inference analysis of step (e), is performed using a plurality of MR based methods, and each NP-cluster selected in the step (e), has negative causal effect on the second disease based on at least 43.75% of the MR based methods used. In certain embodiments, the causal inference analysis of step (e), is performed using a plurality of MR based methods wherein the plurality of MR based methods includes IVW, and each NP-cluster selected in the step (e), has negative causal effect on the second disease based at least on IVW.

Step (f) can include functionally annotating i) one or more NP cluster of the subset of NP clusters selected in step (e) and/or ii) gene clusters mapped with the one or more NP cluster of the subset of NP clusters. The mapped gene cluster can be a gene cluster of step (c). A gene cluster containing genes mapped (e.g., as identified in step (b)) to the NPs within a NP cluster, is mapped to the NP cluster, and vice versa. In some embodiments, in step (f), for a respective NP cluster of the subset of NP clusters and/or a respective gene cluster mapped with the respective NP cluster, the functionally annotating comprises (i) overlapping the respective mapped gene cluster (i.e., gene cluster mapped with the respective NP cluster), with one or more gene function signature lists to determine, significant overlap between the respective mapped gene cluster and the one or more gene function signature lists; and (ii) annotating the respective NP cluster and/or the respective mapped gene cluster with one or more functional characterizations, based at least on the significant overlap of the mapped gene cluster. In some embodiments, in step (f), for a respective NP cluster of the subset of NP clusters, the functionally annotating comprises (i) overlapping a gene cluster mapped with the respective NP cluster, with one or more gene function signature lists to determine, significant overlap between the mapped gene cluster (i.e., gene cluster mapped with the respective NP cluster), and the one or more gene function signature lists; and (ii) annotating the respective NP cluster with one or more functional characterizations, based at least on the significant overlap of the mapped gene cluster. In some embodiments, in step (f), for a respective gene cluster mapped with a NP cluster of the subset of NP clusters, the functionally annotating comprises (i) overlapping the respective mapped gene cluster (i.e., gene cluster mapped with the NP cluster), with one or more gene function signature lists to determine, significant overlap between the respective mapped gene cluster and the one or more gene function signature lists; and (ii) annotating the respective mapped gene cluster with one or more functional characterizations, based at least on the significant overlap of the mapped gene cluster. In some embodiments, in step (f), for a respective gene cluster mapped to a NP cluster of the subset of NP clusters, the functionally annotating comprises (i) overlapping the respective gene cluster, with one or more gene function signature lists to determine, significant overlap between the gene cluster and the one or more gene function signature lists; and (ii) annotating the respective gene cluster with one or more functional characterizations, based at least on the significant overlap of the mapped gene cluster. The functional annotations for a gene cluster can be used to interpret a NP cluster containing the NPs mapped to the genes of the gene cluster. The one or more gene function signature lists can contain curated signatures of cell types and/or biological functions. Gene function signature lists can contain of a collection of genes (represented as gene symbols) that have been statistically demonstrated using various metrics to be representative of a cell type and/or function, and genes in gene function signature lists, based on cell type and/or function are grouped into one or more functional characterization groups. The overlap, e.g., in step (f)-(i), can include categorical comparison of gene symbols in a given gene cluster, to gene symbols in a given functional characterization group of a gene function signature list. For a respective gene cluster, the categorical comparison can include findings of gene symbols in the gene cluster, within gene symbols in a gene functional characterization group. The categorical comparisons can be conducted using any suitable technique. In some embodiments, the categorical comparisons is conducted using the Fisher's exact test. The significant overlap between, e.g. between a respective gene cluster and a respective functional characterization group, can have a threshold Fisher's adjusted p value. In certain embodiments, the threshold Fisher's adjusted p value for significant overlap is, <000.1, <0.01, <0.05, <0.1, <0.15, <0.2, <0.25, <0.3. In certain particular embodiments, the threshold Fisher's adjusted p value for significant overlap is <0.3. In certain particular embodiments, the threshold Fisher's adjusted p value for significant overlap is <0.2. In certain particular embodiments, the threshold Fisher's adjusted p value for significant overlap is <0.05. The p value used can account for biological variability. The significant overlap, between a respective gene cluster and a respective functional characterization group, can also satisfy overlap of a threshold minimum number of genes between the respective gene cluster and the respective functional characterization group. In certain embodiments, the threshold minimum number of genes are about 1 genes to about 12 genes. In certain embodiments, the threshold minimum number of genes are about 1 gene to about 12 genes. In certain embodiments, the threshold minimum number of genes are about 1 gene to about 2 genes, about 1 gene to about 3 genes, about 1 gene to about 4 genes, about 1 gene to about 5 genes, about 1 gene to about 6 genes, about 1 gene to about 7 genes, about 1 gene to about 8 genes, about 1 gene to about 9 genes, about 1 gene to about 10 genes, about 1 gene to about 11 genes, about 1 gene to about 12 genes, about 2 genes to about 3 genes, about 2 genes to about 4 genes, about 2 genes to about 5 genes, about 2 genes to about 6 genes, about 2 genes to about 7 genes, about 2 genes to about 8 genes, about 2 genes to about 9 genes, about 2 genes to about 10 genes, about 2 genes to about 11 genes, about 2 genes to about 12 genes, about 3 genes to about 4 genes, about 3 genes to about 5 genes, about 3 genes to about 6 genes, about 3 genes to about 7 genes, about 3 genes to about 8 genes, about 3 genes to about 9 genes, about 3 genes to about 10 genes, about 3 genes to about 11 genes, about 3 genes to about 12 genes, about 4 genes to about 5 genes, about 4 genes to about 6 genes, about 4 genes to about 7 genes, about 4 genes to about 8 genes, about 4 genes to about 9 genes, about 4 genes to about 10 genes, about 4 genes to about 11 genes, about 4 genes to about 12 genes, about 5 genes to about 6 genes, about 5 genes to about 7 genes, about 5 genes to about 8 genes, about 5 genes to about 9 genes, about 5 genes to about 10 genes, about 5 genes to about 11 genes, about 5 genes to about 12 genes, about 6 genes to about 7 genes, about 6 genes to about 8 genes, about 6 genes to about 9 genes, about 6 genes to about 10 genes, about 6 genes to about 11 genes, about 6 genes to about 12 genes, about 7 genes to about 8 genes, about 7 genes to about 9 genes, about 7 genes to about 10 genes, about 7 genes to about 11 genes, about 7 genes to about 12 genes, about 8 genes to about 9 genes, about 8 genes to about 10 genes, about 8 genes to about 11 genes, about 8 genes to about 12 genes, about 9 genes to about 10 genes, about 9 genes to about 11 genes, about 9 genes to about 12 genes, about 10 genes to about 11 genes, about 10 genes to about 12 genes, or about 11 genes to about 12 genes. In certain embodiments, the threshold minimum number of genes are about 1 gene, about 2 genes, about 3 genes, about 4 genes, about 5 genes, about 6 genes, about 7 genes, about 8 genes, about 9 genes, about 10 genes, about 11 genes, or about 12 genes. In certain embodiments, the threshold minimum number of genes are at least about 1 gene, about 2 genes, about 3 genes, about 4 genes, about 5 genes, about 6 genes, about 7 genes, about 8 genes, about 9 genes, about 10 genes, or about 11 genes. In certain embodiments, the threshold minimum number of genes are about 1 gene. In certain embodiments, the threshold minimum number of genes are about 3 genes. The threshold minimum number of genes for a gene cluster being overlapped can depend of the size of the gene cluster, and/or specificity of the functional annotation. The threshold minimum number of genes for different gene clusters may be same or different. Once the overlapping one or more functional characterization groups, for a respective gene cluster is identified (e.g., in step (f)-(i), based on significant overlap), in step (f)-(ii), the NP cluster that is mapped to the respective gene cluster, can be functionally annotated based on the overlapping one or more functional characterization groups. In a non-limiting example, as described in Example 1 and Table 8, gene cluster mapped to the NP-cluster 2 (Table 10 cluster 2; Table 13-2), overlaps with the functional characterization groups Glucocorticoid Receptor Signaling, Clathrin-mediated Endocytosis Signaling, Neutrophil degranulation, Fc-gamma receptor signaling pathway involved in phagocytosis of GO gene function signature list, and the functional annotation of NP-cluster 2 (Table 8 cluster 2, Table 13-2) includes Glucocorticoid Receptor Signaling, Clathrin-mediated Endocytosis Signaling, Neutrophil degranulation, Fc-gamma receptor signaling pathway involved in phagocytosis. All clusters of the subset of NP clusters selected in step (e) may or may not be functionally annotated. Every gene clusters mapped to NP cluster of the subset of NP clusters selected in step (e), may or may not have significant overlap. In certain embodiments, all clusters of the subset of NP clusters selected in step (e) are functionally annotated in step (f). All gene clusters of step (c) may or may not be functionally annotated. In certain embodiments, the one or more gene function signature lists contain AMPEL LuGENE, AMPEL Ancestry, AMPEL Endotype.32, Endotype.kidney, AMPEL tissues (Tis), Biologically Informed Gene Clustering (BIG-C) signature, Gene Ontology (GO) database, Hallmark gene sets, KEGG Pathway Database, Reactome signature, BRETIGEA signature, IPA, EnrichR, or any combination thereof. In certain embodiments, the one or more gene function signature lists contain AMPEL LuGENE, AMPEL Ancestry, AMPEL tissues (Tis), Biologically Informed Gene Clustering (BIG-C) signature, Gene Ontology (GO) database, Ingenuity Pathway Analysis (IPA), EnrichR, or any combination thereof. In certain embodiments, the one or more gene function signature lists contain Biologically Informed Gene Clustering (BIG-C) signature. In certain embodiments, the one or more gene function signature lists contain IPA, and/or EnrichR. The gene function lists, the functional characterization groups (e.g. categories) within the list, and genes with the functional characterization groups for AMPEL Ancestry and BIG-C, are provided in Catalina, Michelle D., et al. “Patient ancestry significantly contributes to molecular heterogeneity of systemic lupus erythematosus.” JCI insight 5.15 (2020); for GO is publicly available at http://geneontology.org/; for BRETIGEA is provided in McKenzie, Andrew T., et al. “Brain cell type specific gene expression and co-expression network architectures.” Scientific reports 8.1 (2018): 1-19; for Hallmark gene sets, KEGG Pathway Database, Reactome signature is publicly available at http://www.gsea-msigdb.org/gsea/msigdb/collections.jsp. IPA is publicly available at https://www.qiagen.com/us/products/discovery-and-translational-research/next-generation-sequencing/informatics-and-data/interpretation-content-databases/ingenuity-pathway-analysis/. EnrichR is publicly available at https://maayanlab.cloud/Enrichr/. In certain embodiments, the one or more NP cluster of the subset of NP clusters selected in step (e), can be annotated using Ingenuity Pathway Analysis method available from QIAGEN

The NPs can be single nucleotide polymorphisms (SNPs), indels, splice variants, structural variants, copy number variants, transposons, or other forms of genetic variation. In certain embodiments, the NPs are single nucleotide polymorphisms (SNPs), indels, splice variants, structural variants, copy number variants, or transposons. In certain embodiments, the NPs are SNPs.

The first disease can be lupus, coronary artery disease (CAD), myocardial infarction, ischemic stroke, coronary atherosclerosis, cardiovascular disease, cardiomyopathy, depression, asthma, chronic obstructive pulmonary disease (COPD), diabetes mellitus, nonalcoholic fatty liver disease, metabolic disorder inflammatory bowel disease, multiple sclerosis (MS), or glomerulonephritis. In certain embodiment, the first disease is lupus. The second disease is different from the first disease, and can be selected from lupus, coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, multiple sclerosis (MS), and glomerulonephritis.

In some embodiments, the first disease is lupus, and the second disease is a disease or condition associated with lupus. The disease or condition associated with lupus may be any known to those of skill in the art, e.g., any known lupus comorbidity. In some embodiments, the second disease is an autoimmune disease. The autoimmune disease may be selected from, e.g., celiac disease, myasthenia gravis, rheumatoid arthritis, scleroderma, autoimmune thyroid disease, and Sjogren's syndrome. In some embodiments, the second disease is a kidney disease. In some embodiments, the kidney disease is lupus nephritis. In some embodiments, the second disease is a cancer. The cancer may be selected from, e.g., a blood cancer, a gastrointestinal cancer, a lung cancer, a reproductive organ or tissue cancer, a genitourinary cancer, a liver cancer, and a skin cancer. In some embodiments, the cancer is a bladder cancer, a cervical cancer, an esophageal cancer, an oropharyngeal cancer, a larynx cancer, a gastric cancer, a hepatobiliary cancer, non-Hodgkin's lymphoma, Hodgkin's lymphoma, a leukemia, multiple myeloma, non-melanoma skin cancer, a renal cancer, a thyroid cancer, or a vagina/vulva cancer.

In certain aspects, the first disease is lupus. In certain aspects, the second disease is CAD. In certain embodiments, the first disease is lupus, and the second disease is CAD.

In certain embodiments, the first disease is lupus, and the NPs are SNPs, and the method can determine shared SNPs and/or shared biological pathways between lupus and a second disease. In certain embodiments, the first disease is lupus, the second disease is CAD, the NPs are SNPs, and the method can determine shared SNPs and/or shared biological pathways between lupus and CAD. Lupus can be any type of lupus including but not limited to systemic lupus erythematosus (SLE), lupus nephritis, cutaneous lupus erythematosus, drug-induced lupus, and neonatal lupus. In certain embodiments, lupus can be SLE. In certain embodiments, lupus can be lupus nephritis.

The datasets, such as first, second, and third data set, can contain listing of plurality of NPs and their corresponding p-value value for association with a trait of interest, such as first disease (e.g., for the first and second data set) and second disease (e.g., for the third data set). In certain embodiments, the dataset, such as first, second, and third data set, can be data sets containing summary statistics of association of plurality of NPs with a trait of interest, such as first disease (e.g., for the first and second dataset) and second disease (e.g., for the third data set). In certain embodiments, the first dataset can be a data set containing listing of a first plurality of NPs with or without their corresponding p-value value for association with the first disease. In certain embodiments, the first dataset contains listing of a first plurality of NPs and their corresponding p-value value for association with the first disease. In certain embodiments, the first data set is summary statistics from a Genome-Wide Association Studies (GWAS), summary statistics from Immunochip, a dataset obtained from the phenoScanner database, or any combination thereof. The second dataset can contain data regarding summary statistics of association of a second plurality of NPs with the first disease. In certain embodiments, the second dataset is a GWAS and/or Immunochip dataset. The third dataset can contain data regarding summary statistics of association a third plurality of NPs with the second disease. In certain embodiments, the third dataset is a GWAS and/or Immunochip dataset. The first dataset and second dataset can be same or different. In certain aspects, the first disease is lupus, and the first dataset is a SLE GWAS summary statistics, SLE Immunochip summary statistics, SLE data exported from the phenoscanner database, or any combination thereof. In certain aspects, the first disease is lupus, and the second dataset is a SLE GWAS and/or Immunochip dataset. In certain aspects, the second disease is CAD, and the third dataset is a CAD GWAS and/or Immunochip dataset. In certain embodiments, the SLE GWAS dataset is GCST003155. In certain embodiments, the CAD GWAS dataset is GCST004280, GCST000998, GCST001479, GCST005194, GCST005195, or any combination thereof.

The method can be used to categorize patients with respect to the gene clusters(s) and/or NP cluster(s) that capture a majority of their heritability for the first and/or second disease by identifying the NPs the patient contains and comparing them to the gene clusters and/or NP clusters. In certain embodiments, the method includes determining a treatment for the first and/or second disease, wherein the treatment targets one or more genes on the shared biological pathways determined in step (f). In certain embodiments, the method includes determining presence of the one or more shared NPs in a biological sample from a patient, and a determining and categorizing a risk of the first and/or second disease in the patient. In certain embodiments, the method includes providing the treatment to the patient. Treatments provided can be based on presence of a one or more of NPs with respect to a specific cluster, and/or high ratio of risk-NPs (e.g., positive causal NPs) to protective-NPs (negative causal NPs) in the biological sample. Treatments provided can also be based on a gene, gene cluster, or pathway burden scores calculated from the relevant NPs present in the biological sample. Treatments/drugs may target genes and/or biological pathways associated (see for example, Table 13) with the specific cluster, genes or biological pathways upstream of or related to target genes and/or biological pathways, and/or the risk NPs.

In certain embodiments, the method includes diagnosis of the second disease in a patient, wherein the method comprises detecting presence of the one or more of the shared NPs (e.g., NPs within the shared NP clusters) in a biological sample from the patient. The patient can be determined to have the second disease, or can be determined to be at risk of developing the second disease, when the NPs detected/present in the biological sample comprises a higher number of positive causal NPs, compared to negative shared NPs in a biological sample from the patient. The patient can have the first disease. In certain embodiments, the method includes selecting, recommending and/or administering a treatment to the patient based on the presence of the one or more shared NPs in the biological sample. In certain embodiments, the method includes selecting, recommending and/or administering a treatment to the patient when the NPs detected/present in the biological sample comprises a higher number of positive causal NPs, compared to negative causal NPs in a biological sample from the patient. The treatment can be a treatment for the second disease. In certain embodiments, the treatment targets a biological pathway associated with a shared NP detected in the biological sample. In certain embodiments, the method includes diagnosis of the second disease in a patient, wherein the method comprises detecting presence of one or more of the positive causal shared NPs in a biological sample from the patient. In certain embodiments, the method includes diagnosis of the second disease in a patient, wherein the method comprises detecting presence of a higher number of positive causal shared NPs, compared to negative causal shared NPs in a biological sample from the patient. The patient can have the first disease. In certain embodiments, the method includes selecting, recommending and/or administering a treatment to the patient based on the presence of the one or more positive causal shared NPs in the biological sample. In certain embodiments, the method includes selecting, recommending and/or administering a treatment to the patient based on the presence of a higher number of positive causal shared NPs, compared to negative causal shared NPs in the biological sample. The treatment can be a treatment for the second disease. In certain embodiments, the treatment targets a biological pathway associated with a shared NP detected in the biological sample. A NP within a positive causal shared NP cluster is a positive causal NP, and a NP within a negative causal shared NP cluster is a negative causal NP. In certain embodiments, the method includes determining whether the patient has one or more symptoms of the second disease. In certain embodiments, the method includes selecting, recommending, and/or administering the treatment for the second disease to the patient, when the patient has one or more symptoms of the second disease, and the NPs detected/present in the biological sample comprises a higher number of positive causal NPs compared to negative causal NPs. In certain embodiments, the method includes recommending, performing with and/or administering one or more lifestyle changes for the second disease to the patient, when the patient does not have one or more symptoms of the second disease, and the NPs detected/present in the biological sample comprises a higher number of positive causal NPs compared to negative causal NPs. In certain embodiments, the first disease is lupus, and second disease is CAD. In certain embodiments, the second disease is CAD, and the treatment for CAD can a treatment for CAD mentioned below. In certain embodiments, the second disease is CAD, and the lifestyle change for CAD can a lifestyle change mentioned below.

In certain embodiments, the method further includes step (a′), wherein the step (a′) includes performing a causal inference analysis of one or more NPs within the first set of NPs selected in step (a), to select a subset of NPs from the first set of NPs, and in step (b) the one or more NPs of the subset of NPs selected in step (a′) are mapped to their associated E-, T-, C-, and/or P-genes, to identify the plurality of NP-mapped genes. In certain embodiments, the method includes step (a′), and NPs of the subset of NPs selected in step (a′) are mapped to their associated E-, T-, C-, and/or P-genes, to identify the plurality of NP-mapped genes. In certain embodiment, the method excludes step (a′). In certain embodiments, the causal inference analysis of step (a′) includes causal analysis on a third disease. In certain embodiments, one or more NPs within the subset of NPs selected in step (a′), independently, has a positive or a negative causal effect on the third disease. In certain embodiments, one or more NP within the subset of NPs selected in step (a′), has a positive causal effect on the third disease. In certain embodiments, one or more NP within the subset of NPs selected in step (a′), has a negative causal effect on the third disease.

In certain embodiments, each NP within the subset of NPs selected in step (a′), independently, has a positive or a negative causal effect on the third disease. In certain embodiments, each NP within the subset of NPs selected in step (a′), has a positive causal effect on the third disease. In certain embodiments, each NP within the subset of NPs selected in step (a′), has a negative causal effect on the third disease. In certain embodiments, the p-value for the positive or negative causal estimate of each NPs within the subset of NPs, on the third disease is below 0.1, below 0.08, below 0.06, below 0.05, below 0.01, below 0.005, below 0.001, below 0.0005, or below 0.0001. In certain embodiments, the p-value for the positive or negative causal estimate of each NPs within the subset of NPs, on the third disease is below about 0.05. In certain embodiments, the p-value for the positive causal estimate of each NPs within the subset of NPs, on the third disease is below 0.1, below 0.08, below 0.06, below 0.05, below 0.01, below 0.005, below 0.001, below 0.0005, or below 0.0001. In certain embodiments, the p-value for the negative causal estimate of each NPs within the subset of NPs, on the third disease is below 0.1, below 0.08, below 0.06, below 0.05, below 0.01, below 0.005, below 0.001, below 0.0005, or below 0.0001. In certain embodiments, the p-value for the positive causal estimate of each NPs within the subset of NPs, on the third disease is below about 0.05. In certain embodiments, the p-value for the negative causal estimate of each NPs within the subset of NPs, on the third disease is below about 0.05. In certain embodiments, the causal inference analysis in step (a′) includes a single-NP Mendelian randomization (MR) method and/or a Wald-ratio method. In certain embodiments, the causal inference analysis in step (a′) includes a single-NP Mendelian randomization (MR) method. In certain embodiments, the single-NP MR method and/or a Wald-ratio method of step (a′) includes determining causal effect of the one or more NPs of the first set of NPs selected in step (a), individually, on the third disease, where NPs having positive or negative causal effect on the third disease are selected to form the subset of NPs. In certain embodiments, the single-NP MR method and/or a Wald-ratio method of step (a′) includes determining causal effect of the one or more NPs of the first set of NPs selected in step (a), individually, on the third disease, where NPs having positive causal effect on the third disease are selected to form the subset of NPs. In certain embodiments, the single-NP MR method and/or a Wald-ratio method of step (a′) includes determining causal effect of the one or more NPs of the first set of NPs selected in step (a), individually, on the third disease, where NPs having negative causal effect on the third disease are selected to form the subset of NPs. In certain embodiments, NPs of the first set of NPs having the desired the p-value for the positive or negative causal estimate on the third disease is selected to form the subset of NPs of step (a′). In certain embodiments, in the single-NP Mendelian randomization (MR) method, for a respective NP, the causal effect of the NP on the third disease is determined by using the NP individually as instrument variable, summary statistics from a fourth dataset as exposure, and a fifth dataset as outcome. The fourth dataset can include data regarding summary statistics of association a fourth plurality of NPs with the first disease. The fourth data set can be same or different than the first dataset. In certain embodiments, the first data set and the fourth data set are same. In certain embodiments, the first data set and the fourth data set are different. The fourth data set can be same or different than the second dataset. The fifth dataset can include data regarding association of a fifth plurality of NPs with the third disease. In certain embodiments, the fifth plurality of NPs overlap at least partially with the first plurality of NPs, e.g., at least a portion of the NPs listed within the first dataset are also listed in the fifth dataset, and vice versa. In certain embodiments, the fifth plurality of NPs overlap at least partially with the fourth plurality of NPs, e.g., at least a portion of the NPs listed within the fourth dataset are also listed in the fifth dataset, and vice versa. In certain embodiments, the NPs are selected to form the subset of NPs, based on the p-value of the positive or negative causal effect on the third disease. In certain embodiments, the p-value for the positive or negative causal effect on the third disease of the NPs selected to form the subset of NPs is below 0.1, below 0.08, below 0.06, below 0.05, below 0.01, below 0.005, below 0.001, below 0.0005, or below 0.0001. In certain embodiments, the p-value for the positive or negative causal effect on the third disease of the NPs selected to form the subset of NPs is below about 0.05. In certain embodiments, the p-value for the positive causal effect on the third disease of the NPs selected to form the subset of NPs is below 0.1, below 0.08, below 0.06, below 0.05, below 0.01, below 0.005, below 0.001, below 0.0005, or below 0.0001. In certain embodiments, the p-value for the positive causal effect on the third disease of the NPs selected to form the subset of NPs is below about 0.05. In certain embodiments, the p-value for the negative causal effect on the third disease of the NPs selected to form the subset of NPs is below 0.1, below 0.08, below 0.06, below 0.05, below 0.01, below 0.005, below 0.001, below 0.0005, or below 0.0001. In certain embodiments, the p-value for the negative causal effect on the third disease of the NPs selected to form the subset of NPs is below about 0.05.

In certain embodiments, the subset of NPs selected in step (a′), contains one or more groups of NPs, wherein each group independently have a positive or negative causal effect on the third disease. In certain embodiments, the subset of NPs selected in step (a′), contains one or more groups of NPs, wherein each group independently have a positive causal effect on the third disease. In certain embodiments, the subset of NPs selected in step (a′), contains one or more groups of NPs, wherein each group independently have a negative causal effect on the third disease. In certain embodiments, the p-value for the positive or negative causal estimate for each of the one or more groups of NPs within the subset of NPs, on the third disease is below 0.1, below 0.08, below 0.06, below 0.05, below 0.01, below 0.005, below 0.001, below 0.0005, or below 0.0001. In certain embodiments, the p-value for the positive or negative causal estimate for each of the one or more groups of NPs within the subset of NPs, on the third disease is below about 0.05. In certain embodiments, the p-value for the positive causal estimate for each of the one or more groups of NPs within the subset of NPs, on the third disease is below 0.1, below 0.08, below 0.06, below 0.05, below 0.01, below 0.005, below 0.001, below 0.0005, or below 0.0001. In certain embodiments, the p-value for the positive causal estimate for each of the one or more groups of NPs within the subset of NPs, on the third disease is below about 0.05. In certain embodiments, the p-value for the negative causal estimate for each of the one or more groups of NPs within the subset of NPs, on the third disease is below 0.1, below 0.08, below 0.06, below 0.05, below 0.01, below 0.005, below 0.001, below 0.0005, or below 0.0001. In certain embodiments, the p-value for the negative causal estimate for each of the one or more groups of NPs within the subset of NPs, on the third disease is below about 0.05. In certain embodiments, the NPs of the first set of NPs selected in step (a), are grouped into one or more NP-groups, and the causal inference analysis of step (a′) includes, analyzing causal effect of the NP-groups of the first set of NPs, on the third disease, where NP group(s) having positive or negative causal effect on the third disease is(are) selected to form the subset of NPs. In certain embodiments, the NPs of the first set of NPs selected in step (a), are grouped into one or more NP-groups, and the causal inference analysis of step (a′) includes, analyzing causal effect of the NP-groups of the first set of NPs, on the third disease, where NP group(s) having positive causal effect on the second disease is(are) selected to form the subset of NPs. In certain embodiments, the NPs of the first set of NPs selected in step (a), are grouped into one or more NP-groups, and the causal inference analysis of step (a′) includes, analyzing causal effect of the NP-groups of the first set of NPs, on the third disease, where NP group(s) having negative causal effect on the second disease is(are) selected to form the subset of NPs. In certain embodiments, in step (a′) the causal effect of the NP-groups of the first set of NPs, on the third disease can be performed using Mendelian randomization method, where for causal analysis of a respective NP-group of the first set of NPs, at least 2 NPs within the NP-group collectively are used as instrument variable, summary statistics from the fourth dataset is used as exposure, and a fifth dataset is used as outcome. The fourth dataset can include data regarding summary statistics of association a fourth plurality of NPs with the first disease. The fourth data set can be same or different than the first dataset. In certain embodiments, the first data set and the fourth data set are same. In certain embodiments, the first data set and the fourth data set are different. The fourth data set can be same or different than the second dataset. The fifth dataset can include data regarding association of a fifth plurality of NPs with the third disease. In certain embodiments, the fifth plurality of NPs overlap at least partially with the first plurality of NPs, e.g., at least a portion of the NPs listed within the first dataset are also listed in the fifth dataset, and vice versa. In certain embodiments, the fifth plurality of NPs overlap at least partially with the fourth plurality of NPs, e.g., at least a portion of the NPs listed within the fourth dataset are also listed in the fifth dataset, and vice versa. In certain embodiments, in step (a′), for causal analysis of a respective NP-group of the first set of NPs, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP-group, collectively are used as the instrument variable. In certain embodiments, in step (a′), for causal analysis, independently for each NP-group of the first set of NPs, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 or all, NPs within the NP-group, collectively are used as the instrument variable, wherein number of NPs used for different NP groups can be same or different. In certain embodiments, for the causal analysis of step (a′), the NPs within the first set of NPs are grouped according to the chromosomal location. For, example, in a non-limiting example NPs with each separate chromosome, can form separate groups, e.g., NPs within chromosome 1 can be grouped together, NPs within chromosome 2 can be grouped together, NPs within chromosome 3 can be grouped together, and the like. In certain non-limiting examples, NPs within known chromosomal regions can be grouped together, e.g., NPs within non-HLA region can be grouped together. The chromosomal non-HLA region can exclude chromosomal HLA (chromosome 6 short arm), and/or chromosomal extended HLA region (chromosome 6:27-34 Mb). In certain embodiments, in step (a′), the NPs groups are selected to form the subset of NPs based on the p-value of the positive or negative causal effect on the third disease. In certain embodiments, the p-value for the positive or negative causal effect on the third disease of the NP-group(s) that are selected to form the subset of NPs of step (a′), is below 0.1, below 0.08, below 0.06, below 0.05, below 0.01, below 0.005, below 0.001, below 0.0005, or below 0.0001. In certain embodiments, the p-value for the positive or negative causal effect on the third disease of the NP-group(s) that are selected to form the subset of NPs of step (a′), is below 0.05. In certain embodiments, the p-value for the positive causal effect on the third disease of the NP-group(s) that are selected to form the subset of NPs of step (a′), is below 0.1, below 0.08, below 0.06, below 0.05, below 0.01, below 0.005, below 0.001, below 0.0005, or below 0.0001. In certain embodiments, the p-value for the positive causal effect on the third disease of the NP-group(s) that are selected to form the subset of NPs of step (a′) is below 0.05. In certain embodiments, the p-value for the negative causal effect on the third disease of the NP-group(s) that are selected to form the subset of NPs of step (a′), is below 0.1, below 0.08, below 0.06, below 0.05, below 0.01, below 0.005, below 0.001, below 0.0005, or below 0.0001. In certain embodiments, the p-value for the negative causal effect on the third disease of the NP-group(s) that are selected to form the subset of NPs of step (a′) is below 0.05. In certain embodiments, in step (a′) NPs having confounding effect on the third disease are removed. In certain embodiments, in step (a′) NPs having confounding effect on the third disease are removed prior to the causal effect analysis of the NPs the first set of NPs. In certain embodiments, in step (a′) NPs having confounding effect on the third disease are removed prior to the causal effect analysis of the NP-groups of the first set of NPs. The NPs having confounding effect on the third disease, can include NPs with known pleiotropic associations (such as p<1*10⁻⁵) with the third disease, and/or NPs associated with known risk factors of the third disease. In certain embodiments, in step (a′) NPs having confounding effect on the third disease are not removed. The Mendelian randomization of step (a′), such as for causal effect analysis of individual NPs or NP groups, can be performed using any suitable Mendelian randomization methods, including but not limited to inverse-weighted (IVW), IVW-random effects, IVW-fixed effects, simple mode, simple mode-NOME, weighted mode, weighted mode-NOME, simple median, weighted median, penalized weighted median, two sample maximum likelihood, Maximum likehoods, RAPS, Egger, Egger-bootstrap, PRESSO-raw, PRESSO-OC, or any combination thereof.

The third disease can be different from the first disease. The third disease can be same or different from the second disease. In certain embodiment, the second and the third disease are the same. In certain embodiment, the second and the third disease are different. The fifth dataset can be same or different than the third dataset. In certain embodiment, the second and the third disease are the same, and the fifth dataset and third dataset are same. In certain embodiment, the second and the third disease are the same, and the fifth dataset and third dataset are different. In certain embodiment, the second and the third disease are different, and the fifth dataset and third dataset are different. The fourth dataset can be a GWAS dataset. The fifth dataset can be a GWAS dataset.

In certain embodiments, the third disease is different from the first disease, and be can be lupus, coronary artery disease (CAD), cardiovascular myocardial infraction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis. In certain embodiments, the first disease is lupus, and the second and third disease are same and is CAD. In certain embodiments, the first disease is lupus, and the third disease is CAD, and the second disease is myocardial infraction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis.

The method can be used to categorize patients with respect to the gene clusters(s) and/or NP cluster(s) that capture a majority of their heritability for the first and/or second disease by identifying the NPs the patient contains and comparing them to the gene clusters and/or NP clusters. In certain embodiments, the method includes determining a treatment for the first and/or second disease, wherein the treatment targets one or more genes on the shared biological pathways determined in step (f). In certain embodiments, the method includes determining presence of the one or more shared NPs in a biological sample from a patient, and a determining and categorizing a risk of the first and/or second disease in the patient. In certain embodiments, the method includes providing the treatment to the patient. Treatments provided can be based on presence of a one or more of NPs with respect to a specific cluster, and/or high ratio of risk-NPs (e.g., positive causal NPs) to protective-NPs (negative causal NPs) in the biological sample. Treatments provided can also be based on a gene, gene cluster, or pathway burden scores calculated from the relevant NPs present in the biological sample. Treatments/drugs may target genes and/or biological pathways associated (see for example, Table 13) with the specific cluster, genes or biological pathways upstream of or related to target genes and/or biological pathways, and/or the risk NPs.

Certain embodiments are directed to a method for determining the second disease state of a patient. The method can include detecting one or more shared NPs between the first disease and the second disease, in a biological sample from the patient; and determining the second disease state of the patient, based on the presence of the one or more shared NPs in the biological sample. The one or more shared NPs between the first disease and second disease can be determined using a method, e.g., a method containing steps (a), (b), (c), (d), (e), and/or (f), as described above and elsewhere herein. The first disease and the second disease, can be as described herein. The NPs can be as described herein. In certain embodiments, the NPs are SNPs.

Determining the second disease state of the patient can include, determining whether the patient has the second disease, severity of the second disease in the patient, type of the second disease in the patient, and/or whether the patient is at risk of developing the second disease. Determining that the patient is at risk of developing the second can include determining the type of, and/or severity of the second disease the patient is at risk of developing. In certain embodiments, determining the second disease state of the patient include, determining whether the patient has the second disease, or whether the patient is at risk of developing the second disease. Detecting the one or more shared NPs (e.g., between the first disease and second disease), in the biological sample can include detecting whether the one or more NPs are present in the biological sample.

The one or more shared NPs can include the NPs within the positive causal NP clusters, and the NPs within the negative clusters (e.g., as selected in step (e)). Detecting the one or more shared NPs in the biological sample, can include detecting one to all, or any range or value there between, of the shared NPs, in the biological sample. In certain embodiments, detecting the one or more shared NPs in the biological sample, can include detecting i) one to all, or any range or value there between NPs selected from the NPs listed within the positive causal clusters (e.g., clusters having positive causal effect on the second disease) in the biological sample, and/or ii) one to all, or any range or value there between NPs selected from the NPs listed within the negative causal clusters (e.g., clusters having negative causal effect on the second disease) in the biological sample. In certain embodiments, detecting the one or more shared NPs in the biological sample, can include detecting one to all, or any range or value there between NPs selected from the NPs listed within the positive causal clusters (e.g., clusters having positive causal effect on the second disease) in the biological sample. In certain embodiments, detecting the one or more shared NPs in the biological sample include detecting i) at least 1 NP selected from the NPs listed in each of one or more of the positive causal NP clusters, and/or ii) at least 1 NP selected from the NPs listed in each of one or more of the negative causal NP clusters, in the biological sample, wherein the number of NPs selected from different NP clusters can be same or different. In certain embodiments, detecting the one or more shared NPs in the biological sample include detecting i) at least 1 NP selected from the NPs listed in each of the positive causal NP clusters, and/or ii) at least 1 NP selected from the NPs listed in each of the negative causal NP clusters, in the biological sample, wherein the number of NPs selected from different NP clusters can be same or different. In certain embodiments, detecting the one or more shared NPs in the biological sample include detecting i) one to all, or any range or value there between, NPs selected from the NPs listed in each of one or more of the positive causal NP clusters, and/or ii) one to all, or any range or value there between, NPs selected from the NPs listed in each of one or more of the negative causal NP clusters, in the biological sample, wherein the number of NPs selected from different NP clusters can be same or different. In certain embodiments, detecting the one or more shared NPs in the biological sample include detecting i) one to all, or any range or value there between, NPs selected from the NPs listed in each of the positive causal NP clusters, and/or ii) one to all, or any range or value there between, NPs selected from the NPs listed in each of the negative causal NP clusters, in the biological sample, wherein the number of NPs selected from different NP clusters can be same or different. In certain embodiments, detecting the one or more shared NPs in the biological sample include of at least 1 NP selected from the NPs listed in each of one or more of the positive causal NP clusters, in the biological sample, wherein the number of NPs selected from different NP clusters can be same or different. In certain embodiments, detecting the one or more shared NPs in the biological sample include detecting at least 1 NP selected from the NPs listed in each of the positive causal NP clusters, in the biological sample, wherein the number of NPs selected from different NP clusters can be same or different. In certain embodiments, detecting the one or more shared NPs in the biological sample include detecting one to all, or any range or value there between, NPs selected from the NPs listed in each of one or more of the positive causal NP clusters in the biological sample, wherein the number of NPs selected from different NP clusters can be same or different. In certain embodiments, detecting the one or more shared NPs in the biological sample include detecting one to all, or any range or value there between, NPs selected from the NPs listed in each of the positive causal NP clusters, in the biological sample, wherein the number of NPs selected from different NP clusters can be same or different. Detecting the one or more shared NPs (e.g., between the first disease and second disease), in the biological sample can include detecting presence of the one or more NPs in the biological sample. NPs within a NP cluster can be the NPs listed in the cluster.

In certain embodiments, the patient is determined to have the second disease, or is at risk of developing the second disease, when the one or more shared NPs are present in the biological sample. In certain embodiments, the patient is determined to have the second disease, or is at risk of developing the second disease, when the one or more shared NPs selected from the NPs listed within the positive causal clusters are present in the biological sample. In certain embodiments, the patient is determined to have the second disease, or is at risk of developing the second, when a high proportion of NPs listed in the at least one positive causal NP cluster, are present in the biological sample; or higher number of risk NPs compared to protective NPs are present in the biological sample; or both. NPs within the positive causal clusters are risk NPs, and NPs within the negative causal clusters are protective NPs. In certain embodiments, the patient is determined to have the second disease, or is at risk of developing the second disease, when the one or more NPs detected (e.g., present in the biological sample) comprises one or more NPs listed in the positive causal clusters. In certain embodiments, the patient is determined to have the second disease, or is at risk of developing the second disease, when the one or more NPs detected (e.g., present in the biological sample) comprises higher number of risk NPs compared to protective NPs

In certain embodiments, a second disease risk score for the patient is calculated based on presence of the one or more shared NPs in the biological sample, and the second disease state of the patient is determined based on the second disease risk score.

Detecting the one or more shared NPs, in the biological sample can include detecting whether the one or more shared NPs are present in the biological sample. The one or more shared NPs in the biological sample can be detected, e.g., whether the one or more shared NPs are present in the biological sample can be detected, based on analyzing at least a portion of the nucleic acid of the patient in the biological sample. The nucleic acid can be DNA and/or RNA. Analyzing at least a portion of the nucleic acid of the patient, can include analyzing at least a portion of RNA and/or at least a portion of DNA of the patient, in the biological sample. In certain embodiments, analyzing at least a portion of the nucleic acid of the patient in the biological sample includes analyzing at least a portion of the DNA of the patient in the biological sample. Analyzing at least a portion of the DNA of the patient can include sequencing at least a portion of the DNA of the patient. In certain embodiments, analyzing at least a portion of the nucleic acid of the patient in the biological sample can include sequencing at least a portion of the DNA of the patient in the biological sample. In certain embodiments, analyzing at least a portion of the nucleic acid of the patient in the biological sample can include sequencing the DNA of the patient in the biological sample. The DNA can be sequenced using any known method in the art including but not limited to Sanger sequencing, next-generation sequencing, capillary electrophoresis, fragment analysis, or any combination thereof. In certain embodiments, analyzing at least a portion of the nucleic acid of the patient in the biological sample includes analyzing at least a portion of the RNA of the patient in the biological sample. Analyzing at least a portion of the RNA of the patient can include sequencing and/or quantifying at least a portion of the RNA of the patient. In certain embodiments, analyzing at least a portion of the nucleic acid of the patient in the biological sample can include sequencing and/or quantifying at least a portion of the RNA of the patient in the biological sample. In certain embodiments, analyzing at least a portion of the nucleic acid of the patient in the biological sample can include sequencing and/or quantifying the RNA of the patient in the biological sample. RNA can be any as desired to be analyzed by one of skill in the art e.g., total RNA, mRNA, poly A RNA, non-coding RNA, etc. Incertain embodiments, the method includes analyzing at least a portion of the nucleic acid of the patient in the biological sample. In certain embodiments, the method includes analyzing at least a portion of the nucleic acid of the patient in the biological sample to detect presence of the one or more NPs in the biological sample from the patient. In certain embodiments, analyzing at least a portion of the nucleic acid includes measuring expression of the genes associated with the one or more NPs. The genes associated with a NP, can include the genes mapped to the NP (e.g., as determined in step (b)). In certain embodiments, the genes associated with a NP, can include the E-, C-, T, and/or P-gene associated with the NP. RNA sequencing and quantification, and/or gene expression analysis can be performed using any suitable method including but not limited to RNA sequencing, microarray analysis, RNA-Seq, PCR, northern blotting, fluorescent in situ hybridization, serial analysis of gene expression, tiling arrays or any combination thereof. In certain embodiments, analyzing the nucleic acid includes performing enrichment analysis of the genes associated with the one or more NPs. The enrichment analysis can be performed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment analysis is performed using GSVA. In certain embodiments, the method includes analyzing at least a portion of the nucleic acid of the patient in the biological sample to detect presence of the one or more NPs in the biological sample from the patient, and determining the second disease state of the patient based on the presence of the one or more NPs in the biological sample, wherein the one or more NPs are selected from the NPs within the positive causal NP clusters, and the NPs within the negative causal NP clusters, wherein the patient is determined to have the second disease, or is determined to be at risk of developing the second disease when i) the NPs detected/present in the biological sample comprises higher number of risk NPs compared to protective NPs. In certain embodiments, the method includes analyzing at least a portion of the nucleic acid of the patient in the biological sample, wherein the patient is determined to have the second disease, or is determined to be at risk of developing the second disease when i) the NPs detected/present in the biological sample comprises higher number of risk NPs compared to protective NPs.

The biological sample can be a blood sample, isolated peripheral blood mononuclear cells (PBMCs), tissue biopsy sample, nasal fluid, saliva, urine, stool, or any derivative thereof. In certain embodiments, the biological sample can be a blood sample or any derivative thereof. In certain embodiments, the biological sample can be PBMCs or any derivative thereof. In certain embodiments, the biological sample can be tissue biopsy sample or any derivative thereof. In certain embodiments, the biological sample can be tissue biopsy sample or any derivative thereof. In certain embodiments, the biological sample can be tissue biopsy sample or any derivative thereof. In certain embodiments, the biological sample can be nasal fluid sample or any derivative thereof. In certain embodiments, the biological sample can be saliva sample or any derivative thereof. In certain embodiments, the biological sample can be urine sample or any derivative thereof. In certain embodiments, the biological sample can be stool sample or any derivative thereof.

In certain embodiment, the patient has the first disease. In certain embodiments, the patient does not have the first disease. In certain embodiments, the patient is at an elevated risk of having the first disease. In certain embodiments, the patient is asymptomatic for the first disease. In certain embodiments, the patient is suspected of having the first disease.

In certain embodiments, the method comprises determining one or more symptoms of the second disease in the patient. In certain embodiments, the patient is determined to have the second disease when the one or more shared NPs are present in the biological sample. In certain embodiments, the patient is determined to have the second disease when the one or more shared NPs are present in the biological sample, and the patient has the one or more symptoms of the second disease. In certain embodiments, the patient is determined to have the second disease when one or more NPs listed in the positive causal NP clusters, are present in the biological sample, and/or higher number of risk NPs compared to protective NPs are present in the biological sample. In certain embodiments, the patient is determined to have the second disease when one or more NPs listed in the at least one positive causal NP cluster, are present in the biological sample, and/or higher number of risk NPs compared to protective NPs are present in the biological sample. In certain embodiments, the patient is determined to have the second disease when the NPs detected/present in the biological sample comprises positive causal NPs. In certain embodiments, the patient is determined to have the second disease when the NPs detected/present in the biological sample comprises higher number of risk NPs compared to protective NPs. In certain embodiments, the patient is determined to have the second disease when i) one or more NPs listed in the positive causal NP clusters, are present in the biological sample, and/or higher number of risk NPs compared to protective NPs are present in the biological sample, and ii) the patient has the one or more symptoms of the second disease. In certain embodiments, the patient is determined to have the second disease when i) one or more NPs listed in the at least one positive causal NP cluster, are present in the biological sample, and/or higher number of risk NPs compared to protective NPs are present in the biological sample, and, ii) the patient has the one or more symptoms of the second disease. In certain embodiments, the patient is determined to have the second disease when i) the NPs detected/present in the biological sample comprises positive causal NPs, and ii) the patient has the one or more symptoms of the second disease. In certain embodiments, the patient is determined to have the second disease when i) the NPs detected/present in the biological sample comprises higher number of risk NPs compared to protective NPs, and ii) the patient has the one or more symptoms of the second disease. In certain embodiments, the patient is determined to have the second disease when i) a high proportion of NPs listed in at least one positive causal NP cluster, are present in the biological sample. In certain embodiments, the patient is determined to have the second disease when i) a high proportion of NPs listed in at least one positive causal NP cluster, are present in the biological sample, and/or higher number of risk NPs compared to protective NPs are present in the biological sample, and, ii) the patient has the one or more symptoms of the second disease. In certain embodiments, the patient is determined to be at risk of developing the second disease when i) the one or more shared NPs are present in the biological sample, and ii) one or more symptoms of the second disease are absent in the patient. In certain embodiments, the patient is determined to be at risk of developing the second disease when i) a high proportion of NPs listed in at least one positive causal NP cluster, are present in the biological sample, and/or higher number of risk NPs compared to protective NPs are present in the biological sample, and ii) one or more symptoms of the second disease are absent in the patient. In certain embodiments, the patient is determined to be at risk of developing the second disease when i) one or more NPs listed in the positive causal NP clusters, are present in the biological sample, and/or higher number of risk NPs compared to protective NPs are present in the biological sample and ii) one or more symptoms of the second disease are absent in the patient. In certain embodiments, the patient is determined to be at risk of developing the second disease when i) one or more NPs listed in the at least one positive causal NP cluster, are present in the biological sample, and/or higher number of risk NPs compared to protective NPs are present in the biological sample and ii) one or more symptoms of the second disease are absent in the patient. In certain embodiments, the patient is determined to be at risk of developing the second disease when i) the NPs detected/present in the biological sample comprises positive causal NPs and ii) one or more symptoms of the second are absent in the patient. In certain embodiments, the patient is determined to be at risk of developing the second disease when i) the NPs detected/present in the biological sample comprises higher number of risk NPs compared to protective NPs and ii) one or more symptoms of the second are absent in the patient. In certain embodiments, the patient is determined to be at risk of developing the second disease when i) the NPs detected/present in the biological sample comprises positive causal NPs and ii) the patient does not have any symptom of the second disease. In certain embodiments, the patient is determined to be at risk of developing the second disease when i) the NPs detected/present in the biological sample comprises higher number of risk NPs compared to protective NPs and ii) the patient does not have any symptom of the second disease. The method can determine the severity of, type of the second the patient has, or is at risk of developing, based on the shared NPs present in the biological sample.

In certain embodiments, the method comprises selecting, recommending, and/or administering a treatment to the patient based on the second disease state of the patient. In certain embodiments, the method comprises administering the treatment to the patient, based on the second disease state of the patient. In certain embodiments, the treatment is selected, recommended, and/or administered based on the determination that the patient has the second disease. In certain embodiments, the treatment is administered based on the determination that the patient has the second disease. In certain embodiments, the treatment is selected, recommended, and/or administered based on the determination that the patient is at risk of developing the second disease. In certain embodiments, the treatment is administered based on the determination that the patient is at risk of developing the second disease. In certain embodiments, the treatment is selected, recommended, and/or administered based on i) the presence of the one or more shared NPs in the biological sample from the patient, and/or ii) the patient having one or more symptoms of the second disease. In certain embodiments, the treatment is administered based on i) the presence of the one or more shared NPs in the biological sample from the patient, and/or ii) the patient having one or more symptoms of the second disease, and the method can be directed to treating the second disease. In certain embodiments, the treatment is administered based on i) the presence of the one or more shared NPs in the biological sample from the patient, and ii) the patient having one or more symptoms of the second disease, and the method can be directed to treating the second disease. In certain embodiments, the treatment is administered when the NPs detected/present in the biological sample comprises higher number of risk NPs compared to protective NPs, and/or ii) the patient has one or more symptoms of the second disease, and the method can be directed to treating the second disease. In certain embodiments, the treatment is administered when the NPs detected/present in the biological sample comprises positive causal NPs, and/or ii) the patient has one or more symptoms of the second, and the method can be directed to treating the second disease. The treatment selected, recommended, and/or administered can be based on the one or more shared NPs detected (e.g., present) in the biological sample. In certain embodiments, the treatment administered is based on the one or more shared NPs detected (e.g., present) in the biological sample. In certain embodiments, the treatment is administered i) when one or more shared NPs selected from the NPs listed in the positive causal clusters, are present in the biological sample and/or ii) the patient has one or more symptoms of the second disease. In certain embodiments, the treatment is administered when i) a high proportion of NPs listed in at least one positive causal cluster, is present in the biological sample, and/or ii) the patient has one or more symptoms of the second disease. In certain embodiments, the treatment is administered i) when one or more shared NPs selected from the NPs listed in the positive causal clusters, are present in the biological sample, and ii) the patient has one or more symptoms of the second disease. In certain embodiments, the treatment is administered when i) a high proportion of NPs listed in at least one positive causal cluster, is present in the biological sample, and ii) the patient has one or more symptoms of the second disease.

The treatment selected, recommended, and/or administered can be based on the shared NPs present in the biological sample. The treatment administered can be based on the shared NPs present in the biological sample. In certain embodiments, the treatment is based at least on functional annotation of at least one positive causal cluster; wherein one or more NPs listed in the at least one positive cluster, is present in the biological sample. In certain embodiments, the treatment is based at least on functional annotation (e.g., as determined in step (f)) of at least one positive causal cluster; wherein a high proportion of NPs listed in the at least one positive cluster, is present in the biological sample. Treatments based on a functional annotation of a respective NP cluster may target, i) one or more biological pathways associated with the respective NP cluster, ii) one or more genes associated with the respective NP cluster and/or iii) genes and/or biological pathways upstream of or related to the biological pathways associated with the respective NP cluster. In certain embodiments, the treatment targets one or more genes associated with a positive causal NP cluster, wherein one or more NPs selected from the NPs listed within the positive causal NP cluster are present in the biological sample. Genes associated with a NP cluster are genes mapped to NPs (e.g., as determined in step (b)) within the NP cluster. A high proportion of NPs listed in a NP cluster present in the biological sample can refer at least 20%, 30%, 40%, 50%, 60%, 70%, 80%, 90%, 95%, or 100% of the SNPs listed in the NP cluster are present in the biological sample. The treatment can include one or more treatments of the second disease. In certain embodiments, the treatment is configured to treat the second disease. In certain embodiments, the treatment is configured to reduce severity of the second disease. In certain embodiments, the treatment is configured to reduce a risk of developing the second disease. In certain embodiment, the treatment comprises a pharmaceutical composition. The patient can be a human patient.

In certain embodiments, the patient is determined to be at risk of developing the second disease, when the one or more NPs detected are present in the biological sample, but the patient does not have one or more symptoms of the second disease. In certain embodiments, the patient is determined to be at risk of developing the second disease, when the NPs detected/present in the biological sample comprises higher number of risk NPs compared to protective NPs, but the patient does not have one or more symptoms of the second disease. In certain embodiments, the patient is determined to be at risk of developing the second disease, when the NPs detected/present in the biological sample comprises one or more positive causal NP, but the patient does not have one or more symptoms of the second disease. In certain embodiments, the patient is determined to be at risk of developing the second disease, when the NPs detected/present in the biological sample comprises higher number of risk NPs compared to protective NPs, and the patient does not have any symptom of the second disease. In certain embodiments, the patient is determined to be at risk of developing the second disease, when the NPs detected/present in the biological sample comprises one or more positive causal NP, and the patient does not have any symptom of the second disease. In certain embodiments, the patients is recommended, performed with and/or administered one or more lifestyle changes, when the one or more NPs detected are present in the biological sample. In certain embodiments, the patients is recommended, performed with and/or administered one or more lifestyle changes, when the one or more NPs detected are present in the biological sample, but the patient does not have one or more symptoms of the second disease. In certain embodiments, the patients is recommended, performed with and/or administered one or more lifestyle changes, when the NPs detected/present in the biological sample comprises higher number of risk NPs compared to protective NPs, but the patient does not have one or more symptoms of the second disease. In certain embodiments, the patients is recommended, performed with and/or administered one or more lifestyle changes, when the one or more NPs detected/present in the biological sample comprises one or more positive causal NPs, but the patient does not have one or more symptoms of the second disease. In certain embodiments, the patients is recommended, performed with and/or administered one or more lifestyle changes, when the NPs detected/present in the biological sample comprises higher number of risk NPs compared to protective NPs, and the patient does not have any symptom of the second disease. In certain embodiments, the patients is recommended, performed with and/or administered one or more lifestyle changes, when the one or more NPs detected/present in the biological sample comprises one or more positive causal NPs, and the patient does not have any symptom of the second disease.

The patient can be a human patient.

In certain embodiments, the second disease is CAD. In certain embodiments, the first disease is lupus, and the second disease is CAD. In certain embodiments, the first disease is lupus, the second disease is CAD, and NPs are SNPs. In certain embodiments, the first disease is lupus, the second disease is CAD, and NPs are the shared SNPs between lupus and CAD.

In certain embodiments, the second disease is CAD, and the treatment is a treatment for atherosclerosis. In certain embodiments, the second disease is CAD, and the treatment comprises an anti-IFN antibody such as anifrolumab; an anti-oxidized LDL antibody such as orticumab, an anti-PCSK9 such as alirocumab and/or evolocumab; a JAK inhibitor such as baricitinib and/or tofacitinib; a MTOR inhibitor rapamycin; a MPO inhibitor such as PF-1355; an ACE inhibitor such as captopril; a statin; or any combination thereof. In certain embodiments, the patient has lupus the treatment administered comprises an anti-IFN antibody such as anifrolumab; an anti-oxidized LDL antibody such as orticumab, an anti-PCSK9 such as alirocumab and/or evolocumab; a JAK inhibitor such as baricitinib and/or tofacitinib; a MTOR inhibitor rapamycin; a MPO inhibitor such as PF-1355; or any combination thereof. In certain embodiments, the second disease is CAD, and the one or more lifestyle change recommended, performed with and/or administered are one or more lifestyle changes for CAD as described herein.

An aspect of the present disclosure is directed to a method for determining a coronary artery disease (CAD) state of a patient. The method can include (i) detecting one or more SNPs selected from SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; in a biological sample from the patient; and determining a CAD state of the patient, based on the presence of the one or more SNPs in the biological sample. Determining a CAD state of the patient can include, determining whether the patient has CAD, the severity of the CAD, the type of CAD, and/or whether the patient is at risk of developing CAD. Determining that the patient is at risk of developing CAD can include determining the type of, and/or severity of CAD the patient is at risk of developing. In certain embodiments, determining a CAD state of the patient include, determining whether the patient has CAD, or whether the patient is at risk of developing CAD. The patient is determined to have CAD, or is at risk of developing CAD, when the one or more SNPs are present in the biological sample. The method can determine the severity of, type of CAD the patient has, or is at risk of developing, based on the SNPs present in the biological sample.

In certain embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6. In certain embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66. In some embodiments, the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67. In certain embodiments, the one or more SNPs include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 SNPs. In certain embodiments, the one or more SNPs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451, or any value or range there between, SNPs. In certain embodiments, the one or more SNPs consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 or any value or range there between, SNPs. In certain embodiments, the one or more SNPs comprises 2 to 4,451 SNPs. In certain embodiments, the one or more SNPs comprises 2 to 10, 2 to 50, 2 to 100, 2 to 300, 2 to 100, 2 to 500, 2 to 1,000, 2 to 1,500, 2 to 2,000, 2 to 2,009, 2 to 4,451, 10 to 50, 10 to 100, 10 to 300, 10 to 100, 10 to 500, 10 to 1,000, 10 to 1,500, 10 to 2,000, 10 to 2,009, 10 to 4,451, 50 to 100, 50 to 300, 50 to 100, 50 to 500, 50 to 1,000, 50 to 1,500, 50 to 2,000, 50 to 2,009, 50 to 4,451, 100 to 300, 100 to 100, 100 to 500, 100 to 1,000, 100 to 1,500, 100 to 2,000, 100 to 2,009, 100 to 4,451, 300 to 100, 300 to 500, 300 to 1,000, 300 to 1,500, 300 to 2,000, 300 to 2,009, 300 to 4,451, 100 to 500, 100 to 1,000, 100 to 1,500, 100 to 2,000, 100 to 2,009, 100 to 4,451, 500 to 1,000, 500 to 1,500, 500 to 2,000, 500 to 2,009, 500 to 4,451, 1,000 to 1,500, 1,000 to 2,000, 1,000 to 2,009, 1,000 to 4,451, 1,500 to 2,000, 1,500 to 2,009, 1,500 to 4,451, 2,000 to 2,009, 2,000 to 4,451, or 2,009 to 4,451 SNPs. In certain embodiments, the one or more SNPs comprises at least 2, 10, 50, 100, 300, 100, 500, 1,000, 1,500, 2,000, or 2,009 SNPs. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs selected from each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, and 37, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs selected from each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of 1, 2, 3, 4, 5, 6, 7, 8, 9, 10, 11, 12, 13, 14, 15, 16, 17, 18, 19, 20, 21, 22, 23, 24, 25, or 26, or any range there between Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein number of SNPs selected from different Tables can be same or different. In certain embodiments, the one or more SNPs include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein number of SNPs selected from different Tables can be same or different. As an non-limiting illustrative example, the one or more SNPs include 3 SNPs from a respective Table can denote that method include (i) detecting 3 SNPs from SNPs listed in the respective Table, in a biological sample from the patient; and determining the CAD state of the patient, based on the presence of the 3 SNPs in the biological sample. Detecting the one or more SNPs, in the biological sample can include detecting presence of the one or more SNPs in the biological sample. In certain embodiments, the patient is determined to have CAD, or is at risk of developing CAD, when the one or more SNPs detected (e.g., present in the biological sample) comprises one or more SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67. In certain embodiments, the patient is determined to have CAD, or is at risk of developing CAD, when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. In certain embodiments, the patient is determined to have CAD, or is at risk of developing CAD, when the SNPs detected/present in the biological sample comprise higher number of risk SNPs compared to protective SNPs. In certain embodiments, the patient is determined to have CAD, or is at risk of developing CAD, when a high proportion of SNPs listed in the at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. SNPs within the positive causal clusters (e.g. SNPs within Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67) are risk SNPs, and SNPs within the negative causal clusters (e.g. SNPs within Tables: 13-10; 13-24; 13-25; 13-30; 13-40; 13-45; 13-50; 13-61; and 13-66) are protective SNPs. SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67, include all the SNPs listed in Tables 13-1 to 13-67. As a non-limiting illustrative example, “SNPs listed in Table X and Y” includes x+y SNPs, where Table X contains x SNPs and Table Y contains y SNPs, considering no overlap (e.g., the SNPS are different) exists between x and y SNPs, in the event of overlap, duplicate copies can be excluded from analysis.

The one or more SNPs may or may not include SNPs that are not listed in Tables 13-1 to 13-67. In certain embodiments, the one or more SNPs do not include any SNPs that are not listed in Tables 13-1 to 13-67. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66. In certain embodiments, the one or more SNPs do not include any SNPs that are not listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67.

In certain embodiments, a disease risk score for the patient is calculated based on presence of the one or more SNPs in the biological sample, and the CAD state of the patient is determined based on the disease risk score.

Detecting the one or more SNPs, in the biological sample can include detecting whether the one or more SNPs are present in the biological sample. The one or more SNPs in the biological sample can be detected, e.g., whether the one or more SNPs are present in the biological sample can be detected, based on analyzing at least a portion of the nucleic acid of the patient in the biological sample. Presence of the one or more SNPs in the biological sample can be detected, based on analyzing at least a portion of the nucleic acid of the patient in the biological sample.The nucleic acid can be DNA and/or RNA. Analyzing at least a portion of the nucleic acid of the patient, can include analyzing at least a portion of RNA and/or at least a portion of DNA of the patient, in the biological sample. In certain embodiments, analyzing at least a portion of the nucleic acid includes analyzing at least a portion of the DNA of the patient in the biological sample. In certain embodiments, analyzing at least a portion of the nucleic acid includes analyzing at least a portion of the RNA of the patient in the biological sample. In certain embodiments, analyzing the RNA can include, analyzing mRNA. In certain embodiments, analyzing the nucleic acid includes RNA sequencing. In certain embodiments, analyzing the nucleic acid includes mRNA sequencing. In certain embodiments, analyzing the nucleic acid includes DNA sequencing. In certain embodiments, the method includes analyzing at least a portion of the nucleic acid of the patient in the biological sample. In certain embodiments, the method includes analyzing at least a portion of the nucleic acid of the patient in the biological sample to detect presence of the one or more SNPs in the biological sample from the patient. In certain embodiments, analyzing the at least a portion of nucleic acid includes measuring expression of the genes associated with the one or more SNPs. The genes associated with a SNPs, can include the E-, C-, T, and/or P-gene associated with the SNP. In Tables 13-1 to 13-67, genes associated with the SNPs listed in the Tables are listed. In certain embodiments, analyzing the nucleic acid includes performing enrichment analysis of the genes associated with the one or more SNPs. The enrichment analysis can be performed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment analysis is performed using GSVA.

In certain embodiments, the method includes analyzing at least a portion of the nucleic acid of the patient in the biological sample to detect presence of the one or more SNPs in the biological sample from the patient, and determining the CAD state of the patient based on the presence of the one or more SNPs in the biological sample, wherein the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, and wherein the patient is determined to have CAD, or is determined to be at risk of developing CAD when the SNPs detected/present in the biological sample comprises higher number of risk SNPs compared to protective SNPs.

In certain embodiments, the method includes analyzing at least a portion of the nucleic acid of the patient in the biological sample, wherein the patient is determined to have CAD, or is determined to be at risk of developing CAD when the SNPs detected/present in the biological sample comprises higher number of risk SNPs compared to protective SNPs.

The biological sample can be a blood sample, isolated peripheral blood mononuclear cells (PBMCs), tissue biopsy sample, nasal fluid, saliva, urine, stool, or any derivative thereof. In certain embodiments, the biological sample can be a blood sample or any derivative thereof. In certain embodiments, the biological sample can be PBMCs or any derivative thereof. In certain embodiment, the patient has lupus. In certain embodiments, the patient does not have lupus. In certain embodiments, the patient is at an elevated risk of having lupus. In certain embodiments, the patient is asymptomatic for lupus.

In certain embodiments, the method comprises determining one or more symptoms of CAD in the patient. The one or more symptoms of CAD can include symptoms as understood by one of ordinary skill in the art, or by a physician. Non-limiting symptoms of CAD symptoms can include symptoms identified from echocardiogram, exercise stress test, chest X-ray, cardiac catheterization, etc. In certain embodiments, the patient is determined to have CAD when the one or more SNPs are present in the biological sample. In certain embodiments, the patient is determined to have CAD when the one or more SNPs are present in the biological sample, and the patient has the one or more symptoms of CAD. In certain embodiments, the patient is determined to have CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both. In certain embodiments, the patient is determined to have CAD when i) the SNPs detected/present in the biological sample comprises higher number of risk SNPs compared to protective SNPs and, ii) the patient has the one or more symptoms of CAD. In certain embodiments, the patient is determined to have CAD when i) the SNPs detected/present in the biological sample comprises positive causal SNPs and, ii) the patient has the one or more symptoms of CAD. In certain embodiments, the patient is determined to have CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both, and, ii) the patient has the one or more symptoms of the second disease. In certain embodiments, the patient is determined to be at risk of developing CAD when i) the one or more SNPs are present in the biological sample, and ii) one or more symptoms of CAD are absent in the patient. In certain embodiments, the patient is determined to be at risk of developing CAD when i) one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, are present in the biological sample; or higher number of risk SNPs compared to protective SNPs are present in the biological sample; or both, and ii) one or more symptoms of CAD are absent in the patient. In certain embodiments, the patient is determined to be at risk of developing CAD when i) the SNPs detected/present in the biological sample comprises higher number of risk SNPs compared to protective SNPs and ii) one or more symptoms of CAD are absent in the patient. In certain embodiments, the patient is determined to be at risk of developing CAD when i) the SNPs detected/present in the biological sample comprises positive causal SNPs and ii) one or more symptoms of CAD are absent in the patient. In certain embodiments, the patient is determined to be at risk of developing CAD when i) the SNPs detected/present in the biological sample comprises higher number of risk SNPs compared to protective SNPs and ii) the patient does not have any symptoms of CAD. In certain embodiments, the patient is determined to be at risk of developing CAD when i) the SNPs detected/present in the biological sample comprises positive causal SNPs and ii) the patient does not have any symptoms of CAD. The method can determine the severity of, type of CAD the patient has, or is at risk of developing, based on the SNPs present in the biological sample.

In certain embodiments, the method comprises monitoring the CAD disease state of the patient, wherein the monitoring comprises assessing the CAD disease state of the patient at a plurality of different time points. A difference in the assessment of the CAD disease state of the patient among the plurality of time points can be indicative of one or more clinical indications selected from the group consisting of: (i) a diagnosis of the CAD disease state of the patient, (ii) a prognosis of the CAD disease state of the patient, and (iii) an efficacy or non-efficacy of a course of treatment for treating the CAD disease state of the patient. In certain embodiments, the patient has been administered a treatment, and the method can assess an efficacy or non-efficacy of the treatment, for treating the CAD disease state of the patient.

In certain embodiments, the method comprises selecting, recommending, and/or administering a treatment to the patient, based on the CAD state of the patient. In certain embodiments, the method comprises selecting, recommending, and/or administering a treatment to the patient, based on the CAD state of the patient. In certain embodiments, the treatment is selected, recommended, and/or administered based on the determination that the patient has CAD. In certain embodiments, the treatment is administered based on the determination that the patient has CAD. In certain embodiments, the treatment is selected, recommended, and/or administered based on the determination that the patient is at risk of developing CAD. In certain embodiments, the treatment is administered based on the determination that the patient is at risk of developing CAD. In certain embodiments, the method comprises administering the treatment to the patient, based on the CAD state of the patient. In certain embodiments, the treatment is selected, recommended, and/or administered based on i) the presence of the one or more SNPs in the biological sample from the patient, and/or ii) the patient having one or more symptoms of CAD. In certain embodiments, the treatment is administered based on i) the presence of the one or more SNPs in the biological sample from the patient, and/or ii) the patient having one or more symptoms of CAD, and the method can be directed to treating CAD. In certain embodiments, the treatment is administered when the SNPs detected/present in the biological sample comprises higher number of risk SNPs compared to protective SNPs, and/or ii) the patient has one or more symptoms of CAD, and the method can be directed to treating CAD. In certain embodiments, the treatment is administered based when the SNPs detected/present in the biological sample comprises positive causal SNPs, and/or ii) the patient has one or more symptoms of CAD, and the method can be directed to treating CAD. In certain embodiments, the treatment is administered based on i) the presence of the one or more SNPs in the biological sample from the patient, and ii) the patient having one or more symptoms of CAD, and the method can be directed to treating CAD. The treatment selected, recommended, and/or administered can be based on the one or more SNPs detected (e.g., present) in the biological sample. In certain embodiments, the treatment administered is based on the one or more SNPs detected (e.g., present) in the biological sample.

In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and/or ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and/or ii) the patient has one or more symptoms of CAD. In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and ii) the patient has one or more symptoms of CAD.

The treatment selected, recommended, and/or administered can be based on the SNPs present in the biological sample. The treatment administered can be based on the SNPs present in the biological sample. In certain embodiments, the treatment can be based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein one or more SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein one or more SNPs listed in the Table are present in the biological sample. In certain embodiments, the treatment is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein a high proportion of SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein a high proportion of SNPs listed in the Table are present in the biological sample. Treatments based on a functional annotation of a respective Table may target, i) one or more biological pathways (see for example, Table 13) associated with the respective Table, ii) one or more genes listed in the respective Table and/or iii) genes and/or biological pathways upstream of or related to the biological pathways associated with, gene listed in and/or SNPs listed in the respective Table. The treatment can include one or more treatments of CAD. In certain embodiments, the treatment is configured to treat CAD. In certain embodiments, the treatment is configured to reduce severity of CAD. In certain embodiments, the treatment is configured to reduce a risk of developing CAD. In certain embodiments, the treatment comprises a treatment for atherosclerosis. In certain embodiments, the treatment comprises an anti-IFN antibody such as anifrolumab; an anti-oxidized LDL antibody such as orticumab, an anti-PCSK9 such as alirocumab and/or evolocumab; a JAK inhibitor such as baricitinib and/or tofacitinib; a MTOR inhibitor rapamycin; a MPO inhibitor such as PF-1355; an ACE inhibitor such as captopril; a statin; or any combination thereof. In certain embodiments, the treatment comprises a pharmaceutical composition.

In certain embodiments, the patient is determined to be at risk of developing CAD, when the one or more SNPs detected are present in the biological sample, but the patient does not have one or more symptoms of CAD. In certain embodiments, the patient is determined to be at risk of developing CAD, when the SNPs detected/present in the biological sample comprises higher number of risk SNPs compared to protective SNPs, but the patient does not have one or more symptoms of CAD. In certain embodiments, the patient is determined to be at risk of developing CAD, when the SNPs detected/present in the biological sample comprises one or more positive causal SNPs, but the patient does not have one or more symptoms of CAD. In certain embodiments, the patient is determined to be at risk of developing CAD, when the SNPs detected/present in the biological sample comprises higher number of risk SNPs compared to protective SNPs, and the patient does not have any symptoms of CAD. In certain embodiments, the patient is determined to be at risk of developing CAD, when the SNPs detected/present in the biological sample comprises one or more positive causal SNPs, but the patient does not have any symptoms of CAD. In certain embodiments, the patients is recommended, performed with and/or administered one or more lifestyle changes, when the one or more SNPs detected are present in the biological sample. In certain embodiments, the patients is recommended, performed with and/or administered one or more lifestyle changes, when the one or more SNPs detected are present in the biological sample, but the patient does not have one or more symptoms of CAD. In certain embodiments, the patients is recommended, performed with and/or administered with one or more lifestyle changes, when the SNPs detected/present in the biological sample comprises higher number of risk SNPs compared to protective SNPs, but the patient does not have one or more symptoms of CAD. In certain embodiments, the patients is recommended, performed with and/or administered one or more lifestyle changes, when the SNPs detected/present in the biological sample comprises one or more positive causal SNPs, but the patient does not have one or more symptoms of CAD. In certain embodiments, the patients is recommended, performed with and/or administered with one or more lifestyle changes, when the SNPs detected/present in the biological sample comprises higher number of risk SNPs compared to protective SNPs, but the patient does not any symptoms of CAD. In certain embodiments, the patients is recommended, performed with and/or administered one or more lifestyle changes, when the SNPs detected/present in the biological sample comprises one or more positive causal SNPs, but the patient does not have any symptoms of CAD. The one or more lifestyle changes can include monitoring, such as frequent monitoring the patient for one or more symptoms of CAD. Monitoring can include monitoring through echocardiogram, exercise stress test, chest X-ray, cardiac catheterization, etc, for one or more symptoms of CAD. Frequent monitoring can include a monitoring at a higher frequency, compared to past (e.g., past 1 month, 3 months, 6 months, 1 year, 2 years, 3 years, 5 years, 10 years, etc.) monitoring of the patient. Frequent monitoring can include a monitoring at a higher frequency, compared to the monitoring and/or recommended monitoring of a control subject having similar age, sex, and/or ethnicity as of the patient. In certain embodiments, the SNPs detected/present in the biological sample comprises higher number of risk SNPs compared to protective SNPs, and the method includes i) selecting, recommending, and/or administering the treatment to the patient when the patient has one or more symptoms of CAD, or ii) recommending, performing with and/or administering one or more lifestyle changes when the patient does not have one or more symptoms of CAD.

The patient can be a human patient.

In some embodiments, the method comprises selecting at least one treatment for the patient based on the association of the patient's SNP analysis with a disease or disorder according to the methods of the invention. Selecting a treatment may include recommending a treatment for, administering a treatment to, and/or providing a treatment to the patient. A treatment may be any known in the art as potentially appropriate for treatment of a patient having the disease or disorder, or a condition secondary to the disease or disorder. A treatment may comprise a drug, medical device, surgical procedure, lifestyle modification, physical therapy, psychological counseling, pain management therapy, and/or monitoring of disease, e.g., at increased frequency. A drug may comprise any composition or pharmaceutical, including any known approved or experimental drug, supplement, prebiotic, or probiotic, in any formulation including but not limited to one administered to the patient by any known route including an injection of any kind (e.g., subdermal/subcutaneous, intramuscular, intravenous, intrathecal, inhaled, oral, nasal, topical, patch, implant). A drug may be any known to those of skill in the art as potentially useful for treatment of the disease or disorder. A lifestyle modification may include any behavioral or environmental modification, including modulation of (e.g., increase or decrease as appropriate, in any aspect of), smoking (for cessation or reduction), drug use (for cessation or reduction of recreational or other drugs), sun exposure, infection exposure, weight, physical activity (e.g., to increase cardiovascular fitness or muscle strength through exercise frequency and/or type), diet (e.g., to reduce salt, reduce sugar, eliminate irritating or inflammatory foods, promote weight loss, lower BMI), sleep habits (e.g., to improve sleep quality), stress level (e.g., to decrease through meditation and/or mindfulness), and participation in any program designed to promote such a change. Monitoring may include doctor visits, testing (e.g., exercise stress test, imaging, labs including cholesterol, blood pressure, blood glucose, Alc, inflammatory markers, troponin), and self-monitoring (e.g., weight, waist circumference, blood pressure, blood glucose) to evaluate the development or progression of the disease or disorder or a condition secondary to the disease or disorder.

In some embodiments the treatment is selected based on the presence of a high proportion of NPs with respect to a specific cluster, and/or high ratio of risk-NPs (e.g., positive causal NPs) to protective-NPs (negative causal NPs) in the biological sample. A treatment may target genes and/or biological pathways associated with the specific cluster, for example, the Table 13 clusters, and the risk NPs. In some embodiments, a selected treatment targets a disease or disorder associated with a specific cluster. A disease association with a NP cluster may be designated using any method and resource known to those of skill in the art. In some embodiments, a disease association is designated using a genetic association database. A genetic association database can collate search results from multiple sources, e.g., Elsevier pathway collection, DisGeNET, GWAS catalog, Orphanet, WikiPathway, Ingenuity pathway analysis, KEGG, PheWeb, Jensen DISEASES, PhenGenI Association, GO Biological process, and Human phenotype ontology.

Genetic association databases are known to those of skill in the art and include, as examples, the EnrichR database (Kuleshov M V et al., 2016, Enrichr: a comprehensive gene set enrichment analysis web server 2016 update. Nucleic Acids Res. 44:W90-7, incorporated herein by reference), gprofiler (Reimand J et al, 2007, g:Profiler—a web-based toolset for functional profiling of gene lists from large-scale experiments NAR 35 W193-W200, incorporated herein by reference), clusterProfiler (Wu T et al., 2021, clusterProfiler 4.0: A universal enrichment tool for interpreting omics data, The Innovation, 2(3), 100141, incorporated herein by reference), and ReactomePA.

A selected treatment that targets a disease or disorder associated with a specific cluster can be any known to those of skill in the art for treatment of the associated disease or disorder. In some embodiments, the NP cluster is associated with a disease or disorder selected from myocardial injury, SLE, ischemic stroke, coronary artery disease, X-linked thrombocytopenia, cardiomyopathy, diabetes (e.g., Type I diabetes, Type II diabetes), atherosclerosis, dyslipidemia (e.g., high total cholesterol, high LDL cholesterol, low HDL cholesterol, high triglycerides), ulcerative colitis, inflammatory bowel disease, ischemic heart disease, Sjogren's syndrome, cardiovascular disease, hemolytic anemia, obesity, thyroid disease, angioedema, Behcet syndrome, and chronic inflammatory disease.

In some embodiments, the NP cluster is myocardial injury, and the treatment is selected from one or more treatment for myocardial injury or a condition secondary to myocardial injury known to those of skill in the art, including, e.g.: a drug to prevent further injury, e.g., a treatment for myocardial ischemia, high cholesterol, and/or dyslipidemia, when appropriate, β-blocker, angiotensin-converting enzyme inhibitor (e.g., benazepril, captopril, enalapril, fosinopril, lisinopril, moexipril, perindopril, quinapril, ramipril, trandolapril), angiotensin II receptor blocker (azilsartan, candesartan, eprosartan, irbesartan, losartan, olmesartan, telmisartan, valsartan), statin (e.g., atorvastatin, fluvastatin, lovastatin, pravastatin, rosuvastatin calcium, simvastatin), platelet inhibitor (e.g., ticagrelor, clopidogrel, prasugrel, aspirin, cilostazol); any appropriate treatment described elsewhere herein; any treatment described herein for use when a high proportion of SNPs listed in Table 13-3 is detected in the biological sample; a lifestyle modification (e.g., to diet, smoking, alcohol consumption, weight, physical activity); and increased disease monitoring (e.g., coronary artery calcium, troponin level, EKG, echocardiogram, chest X-ray, CCT, PET, MRI, SPECT, exercise stress test, TEE, MUGA scan, MPI).

In some embodiments, the NP cluster is associated with SLE, and the treatment is selected from one or more treatment for SLE or a condition secondary to SLE known to those of skill in the art, including, e.g.: any approved or experimental lupus drug, e.g., hydroxychloroquine, methotrexate, corticosteroid, an NSAID, an immune suppressant (e.g., azathioprine (Imuran), mycophenolate mofetil (Cellcept), methotrexate, cyclophosphamide (Cytoxan), rituximab, belimumab (Benlysta), Saphnelo (anifrolumab-fnia), Voclosporin); any appropriate treatment described elsewhere herein; any treatment described herein for use when a high proportion of SNPs listed in Table 13-5 or 13-14 is detected in the biological sample; a lifestyle modification (e.g. to diet, smoking, stress, physical activity, sun exposure, exposure to infection); physical therapy; psychological counseling; pain management therapy; and increased disease monitoring (e.g., inflammation markers, ANA, anti-dsDNA, anti-Sm, anti-RNP, anti-Ro/SSA, anti-La/SSB, antiphospholipid antibodies, LAC, aCL, aβ2GPI, autoantibody panel, complement, CBC, ESR, CRP, CMP, urinalysis).

In some embodiments, the NP cluster is associated with ischemic stroke, and the treatment is selected from one or more treatment for ischemic stroke or a condition secondary to ischemic stroke known to those of skill in the art, including, e.g.: any approved or experimental drug to treat or prevent ischemic stroke, e.g., anticoagulant (e.g., apixaban, dabigatran, edoxaban, rivaroxaban, warfarin, thrombolytic (e.g., streptokinase, alteplase, tenecteplase, reteplase, urokinase), platelet inhibitor (e.g., ticagrelor, clopidogrel, prasugrel, aspirin, cilostazol), fibrinolytic (e.g., r-tPA), blood pressure-lowering; any appropriate treatment described elsewhere herein; any treatment described herein for use when a high proportion of SNPs listed in Table 13-5 is detected in the biological sample; a lifestyle modification (e.g. to diet, blood pressure control); thrombectomy or other clot-removal procedure; physical therapy; and increased disease monitoring (e.g., CT, MRI, EEG, evoked response, blood flow test).

In some embodiments, the NP cluster is associated with coronary artery disease/ischemic heart disease, and the treatment is selected from one or more treatment for coronary artery disease/ischemic heart disease or a condition secondary to coronary artery disease/ischemic heart disease known to those of skill in the art, including, e.g.: any approved or experimental drug to treat or prevent coronary artery disease/ischemic heart disease, e.g., nitroglycerin; a beta blocker (e.g., acebutolol, atenolol, betaxolol, bisoprolol/hydrochlorothiazide, bisoprolol, metoprolol, nadolol, propranolol, sotalol); a calcium channel blocker; a thrombolytic (e.g., streptokinase, alteplase, tenecteplase, reteplase, urokinase); any appropriate treatment described elsewhere herein; any treatment described herein for use when a high proportion of SNPs listed in Table 13-5 or 13-23 is detected in the biological sample; a lifestyle modification (e.g., to diet, alcohol consumption, smoking, weight, physical activity, sleep habits, stress level); and increased disease monitoring (e.g., of weight, coronary artery calcium, blood pressure, blood cholesterol, blood glucose, A1c, EKG, echocardiogram, chest X-ray, CCT, PET, MRI, SPECT, exercise stress test, TEE, MUGA scan, MPI).

In some embodiments, the NP cluster is associated with X-linked thrombocytopenia, and the treatment is selected from one or more treatment for X-linked thrombocytopenia or a condition secondary to X-linked thrombocytopenia known to those of skill in the art, including: any approved or experimental drug to treat or prevent X-linked thrombocytopenia, e.g., hematopoietic stem cell transplantation, corticosteroids, intravenous immunoglobulin; any appropriate treatment described elsewhere herein; any treatment described herein for use when a high proportion of SNPs listed in Table 13-13 is detected in the biological sample; a lifestyle modification (e.g. avoiding injury or surgery); and increased disease monitoring (e.g., blood counts, monitoring for infections or malignancies).

In some embodiments, the NP cluster is associated with cardiomyopathy, and the treatment is selected from one or more treatment for cardiomyopathy or a condition secondary to cardiomyopathy known to those of skill in the art, including, e.g.: any approved or experimental drug to treat or prevent cardiomyopathy, e.g., angiotensin-converting enzyme inhibitor (e.g., benazepril, captopril, enalapril, fosinopril, lisinopril, moexipril, perindopril, quinapril, ramipril, trandolapril), angiotensin II receptor blocker (e.g., azilsartan, candesartan, eprosartan, irbesartan, losartan, olmesartan, telmisartan, valsartan), beta blocker (e.g., acebutolol, atenolol, betaxolol, bisoprolol/hydrochlorothiazide, bisoprolol, metoprolol, nadolol, propranolol, sotalol), calcium channel blocker, digoxin, anticoagulant, anti-inflammatory, antiarrhythmic; any appropriate treatment described elsewhere herein; any treatment described herein for use when a high proportion of SNPs listed in Table 13-33 is detected in the biological sample; a lifestyle modification (e.g., to diet, alcohol consumption, smoking, weight, physical activity, sleep, stress); surgery (e.g., septal myectomy, septal ablation, heart transplant); medical device implantation (e.g., pacemaker, CRT, LVAD, ICD); psychological counseling; and increased disease monitoring (e.g., weight, blood pressure, blood cholesterol, blood glucose, A1c, EKG, echocardiogram, chest X-ray, CCT, PET, MRI, SPECT, exercise stress test, TEE, MUGA scan, MPI).

In some embodiments, the NP cluster is associated with diabetes type 1, and the treatment is selected from one or more treatment for diabetes type 1 or a condition secondary to diabetes type 1 known to those of skill in the art, including: any approved or experimental drug to treat or prevent diabetes type 1, e.g., insulin and insulin derivatives, islet cell transplantation; any appropriate treatment described elsewhere herein; any treatment described herein for use when a high proportion of SNPs listed in Table 13-5, 13-33, 13-34, or 13-36 is detected in the biological sample; a lifestyle modification (e.g. to diet, physical activity); and increased disease monitoring (e.g., of blood glucose, A1c, weight, BMI, islet autoimmunity, eye function, cardiovascular function).

In some embodiments, the NP cluster is associated with diabetes type 2, and the treatment is selected from one or more treatment for diabetes type 2 or a condition secondary to diabetes type 2 known to those of skill in the art, including: any approved or experimental drug to treat or prevent diabetes type 2, e.g., Metformin, sulfonylureas, DPP-4 inhibitors, SGLT2 inhibitors, GLP-1 receptor agonists (e.g., semaglutide), Meglitinides, Thiazolidinediones, Alpha-glucosidase inhibitors, insulin; any appropriate treatment described elsewhere herein; any treatment described herein for use when a high proportion of SNPs listed in Table 13-5, 13-33, 13-34, or 13-36 is detected in the biological sample; a lifestyle modification (e.g. to diet, physical activity, smoking, alcohol, blood pressure management, weight, BMI); and increased disease monitoring (e.g., of blood glucose, A1c, weight, BMI, complications including eye function, cardiovascular function).

In some embodiments, the NP cluster is associated with atherosclerosis, and the treatment is selected from one or more treatment for atherosclerosis or a condition secondary to atherosclerosis known to those of skill in the art, including: any approved or experimental drug to treat or prevent atherosclerosis, e.g., a statin (e.g., atorvastatin, fluvastatin, lovastatin, pravastatin, rosuvastatin calcium, simvastatin), a cholesterol absorption inhibitor (e.g., ezetemibe), bile acid sequestrants (e.g., Cholestyramine (Questran®, Questran® Light, Prevalite®, Locholest®, Locholest® Light), Colestipol (Colestid®), Colesevelam Hcl (WelChol®)); PCSK9 inhibitors (e.g., alirocumab, evolocumab); Adenosine triphosphate-citrate lyase (ACL) inhibitors (e.g., Bempedoic acid (Nexletol), Bempedoic acid and ezetimibe (Nexlizet)); other statin combinations (e.g., Caduet® (atorvastatin+amlodipine), Vytorin™ (simvastatin+ezetimibe)); fibrates (e.g., Gemfibrozil (Lopid®), Fenofibrate (Antara®, Lofibra®, Tricor®, Triglide™)), Clofibrate (Atromid-S); niacin; Omega-3 Fatty Acid Ethyl Esters (e.g., Lovaza®, Vascepa™, Epanova®, Omtryg®); Marine-Derived Omega-3 Polyunsaturated Fatty Acids (PUFAs); any appropriate treatment described elsewhere herein; any treatment described herein for use when a high proportion of SNPs listed in Table 13-12, 13-23 or 13-48 is detected in the biological sample; a lifestyle modification (e.g. to diet, physical activity, smoking, alcohol, blood pressure management); medical device implantation (e.g., stent); and increased disease monitoring (e.g., of blood cholesterol, blood lipids, coronary artery calcium).

In some embodiments, the NP cluster is associated with dyslipidemia (including any known abnormality in blood lipid levels, e.g., high LDL cholesterol, low HDL cholesterol, high triglycerides), and the treatment is selected from one or more treatment for dyslipidemia or a condition secondary to dyslipidemia known to those of skill in the art, including: any approved or experimental drug to treat or prevent dyslipidemia, e.g., icosapent ethyl, a statin (e.g., atorvastatin, fluvastatin, lovastatin, pravastatin, rosuvastatin calcium, simvastatin), a cholesterol absorption inhibitor (e.g., ezetemibe), bile acid sequestrants (e.g., Cholestyramine (Questran®, Questran® Light, Prevalite®, Locholest®, Locholest® Light), Colestipol (Colestid®), Colesevelam Hcl (WelChol®)); PCSK9 inhibitors (e.g., alirocumab, evolocumab, inclisiran); Adenosine triphosphate-citrate lyase (ACL) inhibitors (e.g., Bempedoic acid (Nexletol), Bempedoic acid and ezetimibe (Nexlizet)); other statin combinations (e.g., Caduet® (atorvastatin+amlodipine), Vytorin™ (simvastatin+ezetimibe)); fibrates (e.g., Gemfibrozil (Lopid®), Fenofibrate (Antara®, Lofibra®, Tricor®, Triglide™)), Clofibrate (Atromid-S); niacin; Omega-3 Fatty Acid Ethyl Esters (e.g., Lovaza®, Vascepa™, Epanova®, OmtrygR); Marine-Derived Omega-3 Polyunsaturated Fatty Acids (PUFAs); any appropriate treatment described elsewhere herein; any treatment described herein for use when a high proportion of SNPs listed in Table 13-12, 13-18, 13-23 or 13-48 is detected in the biological sample; a lifestyle modification (e.g. to diet, physical activity, smoking, alcohol, blood pressure management); and increased disease monitoring (e.g., of triglycerides, blood cholesterol, blood lipids, coronary artery calcium).

In some embodiments, the NP cluster is associated with ulcerative colitis, and the treatment is selected from one or more treatment for ulcerative colitis or a condition secondary to ulcerative colitis known to those of skill in the art, including: any approved or experimental drug to treat or prevent ulcerative colitis, e.g., aminosalicylates (e.g., sulfasalazine, mesalamine, olsalazine, balsalazide), corticosteroids (e.g., prednisone, prednisolone, methylprednisolone, budesonide), immunomodulators including biologics (e.g., azathioprine, 6-mercaptopurine, cyclosporine, tacrolimus, ozanimod, tofacitinib, upadacitinib, adalimumab, golimumab, infliximab, ustekinumab, vedolizumab), NSAIDs, prebiotics, probiotics; any appropriate treatment described elsewhere herein; any treatment described herein for use when a high proportion of SNPs listed in Table 13-18 is detected in the biological sample; a lifestyle modification (e.g., to diet, stress level, physical activity, sleep, smoking); surgery (e.g., proctocolectomy); pain management therapy; and increased disease monitoring (e.g., infection testing, inflammatory markers, stool analysis, blood count, imaging, e.g., endoscopy, biopsy).

In some embodiments, the NP cluster is associated with inflammatory bowel disease and the treatment is selected from one or more treatment for inflammatory bowel disease or a condition secondary to inflammatory bowel disease known to those of skill in the art, including: any approved or experimental drug to treat or prevent inflammatory bowel disease, e.g., aminosalicylates, antibiotics, corticosteroids, immunomodulators including biologics, NSAIDs, prebiotics, probiotics; any appropriate treatment described elsewhere herein; any treatment described herein for use when a high proportion of SNPs listed in Table 13-18 is detected in the biological sample; a lifestyle modification (e.g., to diet, stress level, physical activity, sleep, smoking); pain management therapy; surgery (e.g., bowel resection, colectomy, proctocolectomy, ileostomy); and increased disease monitoring (e.g., infection testing, inflammatory markers, blood count, stool analysis, imaging, e.g., colonoscopy, upper endoscopy, capsule endoscopy, sigmoidoscopy, endoscopic ultrasound, CT, MRI).

In some embodiments, the NP cluster is associated with Sjogren's syndrome, and the treatment is selected from one or more treatment for Sjogren's syndrome or a condition secondary to Sjogren's syndrome known to those of skill in the art, including: any approved or experimental drug to treat or prevent Sjogren's syndrome, e.g., DMARDS (e.g., hydroxychloroquine, azathioprine, mycophenylate, leflunomide, cyclosporine), cyclophosphamide, biologics (e.g., rituximab, belimumab), NSAIDS, dry eye treatments (e.g., Evoxac® (cevimeline), Salagen® (pilocarpine hydrochloride), NeutraSal®), and dry mouth treatments (e.g., Restasis® (cyclosporine ophthalmic emulsion), Xiidra® (lifitegrast ophthalmic solution), CEQUA™ (cyclosporine ophthalmic solution), TYRVAYA™ (varenicline solution)); any appropriate treatment described elsewhere herein; any treatment described herein for use when a high proportion of SNPs listed in Table 13-23 is detected in the biological sample; a lifestyle modification (e.g., to diet, stress level, physical activity, sleep, smoking); physical therapy; speech therapy, psychological counseling; pain management therapy; and increased disease monitoring (e.g., labs for disease and inflammatory markers, eye tests for tear production, dry spots, dental tests for salivary flow, salivary gland analysis, cancer screening, e.g., for lymphomas, infection testing).

In some embodiments, the NP cluster is associated with cardiovascular disease, and the treatment is selected from one or more treatment for cardiovascular disease or a condition secondary to cardiovascular disease known to those of skill in the art, including: any approved or experimental drug to treat or prevent cardiovascular disease, e.g., nitroglycerin, a beta blocker (e.g., acebutolol, atenolol, betaxolol, bisoprolol/hydrochlorothiazide, bisoprolol, metoprolol, nadolol, propranolol, sotalol), a combined alpha- and beta-blocker (e.g., carvedilol, labetalol hydrochloride), a calcium channel blocker (e.g., amlodipine, diltiazem, felodipine, nifedipine, nimodipine, nisoldipine, verapamil, verelan), a thrombolytic (e.g., streptokinase, alteplase, tenecteplase, reteplase, urokinase), a diuretic, a vasodilator, a drug to prevent further injury, e.g., a treatment for myocardial ischemia, high cholesterol, and/or dyslipidemia, when appropriate, P-blocker, angiotensin-converting enzyme inhibitor (e.g., benazepril, captopril, enalapril, fosinopril, lisinopril, moexipril, perindopril, quinapril, ramipril, trandolapril), angiotensin II receptor blocker (e.g., azilsartan, candesartan, eprosartan, irbesartan, losartan, olmesartan, telmisartan, valsartan), statin (e.g., atorvastatin, fluvastatin, lovastatin, pravastatin, rosuvastatin calcium, simvastatin), angiotensin receptor-neprilysin inhibitor (e.g., sacubitril/valsartan), platelet inhibitor (e.g., ticagrelor, clopidogrel, prasugrel, aspirin, cilostazol), dual antiplatelet therapy, cholesterol absorption inhibitor (e.g., ezetemibe), bile acid sequestrants (e.g., Cholestyramine (Questran®, Questran® Light, Prevalite®, Locholest®, Locholest® Light), Colestipol (Colestid®), Colesevelam Hcl (WelChol®)), PCSK9 inhibitors (e.g., alirocumab, evolocumab), Adenosine triphosphate-citrate lyase (ACL) inhibitors (e.g., Bempedoic acid (Nexletol), Bempedoic acid and ezetimibe (Nexlizet)), other statin combinations (e.g., Caduet® (atorvastatin+amlodipine), Vytorin™ (simvastatin+ezetimibe)), fibrates (e.g., Gemfibrozil (Lopid®), Fenofibrate (Antara®, Lofibra®, Tricor®, Triglide™)), Clofibrate (Atromid-S), niacin, Omega-3 Fatty Acid Ethyl Esters (e.g., Lovaza®, Vascepa™, Epanova®, Omtryg®), Marine-Derived Omega-3 Polyunsaturated Fatty Acids (PUFAs), anticoagulant (e.g., apixaban, dabigatran, edoxaban, rivaroxaban, warfarin, thrombolytic (e.g., streptokinase, alteplase, tenecteplase, reteplase, urokinase), fibrinolytic (e.g., r-tPA), blood pressure-lowering; any appropriate treatment described elsewhere herein, including as described herein for myocardial injury, atherosclerosis, ischemic stroke, coronary artery disease/ischemic heart disease, dyslipidemia, or cardiomyopathy; any treatment described herein for use when a high proportion of SNPs listed in Table 13-28, 13-3, 13-12, 13-23, 13-48, 13-5, or 13-33 is detected in the biological sample; a lifestyle modification (e.g., to diet, alcohol consumption, smoking, weight, physical activity, sleep, stress); surgery (e.g., bypass, angioplasty, carotid endarterectomy, septal myectomy, septal ablation, heart transplant); medical device implantation (e.g., stent, pacemaker, CRT, LVAD, ICD); psychological counseling; and increased disease monitoring (e.g., weight, blood pressure, blood cholesterol, blood glucose, A1c).

In some embodiments, the NP cluster is associated with hemolytic anemia, and the treatment is selected from one or more treatment for hemolytic anemia or a condition secondary to hemolytic anemia known to those of skill in the art, including: any approved or experimental drug to treat or prevent hemolytic anemia, e.g., mitapivat, bone marrow transplant, stem cell transplant, blood transfusion, azathioprine, cyclophosphamide, rituximab, steroid (e.g., triamcinolone, methylprednisolone, dexamethasone, clinacort, kenalog, cortisone), intravenous immunoglobulin; any appropriate treatment described elsewhere herein; any treatment described herein for use when a high proportion of SNPs listed in Table 13-28 is detected in the biological sample; a lifestyle modification (e.g. to diet, physical activity, rest/sleep); surgery, e.g., splenectomy; and increased disease monitoring (e.g., peripheral blood smear, heart monitoring, gallstone treatment or removal).

In some embodiments, the NP cluster is associated with obesity, and the treatment is selected from one or more treatment for obesity or a condition secondary to obesity known to those of skill in the art, including: any approved or experimental drug to treat or prevent obesity, e.g., bupropion-naltrexone, liraglutide, orlistat, phentermine-topiramate; any appropriate treatment described elsewhere herein; any treatment described herein for use when a high proportion of SNPs listed in Table 13-34 is detected in the biological sample; a lifestyle modification (e.g. to diet, physical activity); surgery, e.g, endoscopic sleeve gastroplasty, intragastric balloon, gastric bypass, gastric sleeve, gastric banding; medical device implantation (e.g., vagal nerve blockade); psychological counseling; and increased disease monitoring (e.g., weight, BMI, waist circumference, blood pressure, thyroid function, liver function, diabetes screening, heart function).

In some embodiments, the NP cluster is associated with thyroid disease, and the treatment is selected from one or more treatment for thyroid disease (including but not limited to hyperthyroidism, hypothyroidism, Hashimoto's thyroiditis, Graves' disease, goiter, thyroid nodules, or a condition secondary to thyroid disease known to those of skill in the art, including: any approved or experimental drug to treat or prevent thyroid disease, e.g., antithyroid medication (e.g., methimazole), radioiodine therapy, beta-blockers, thyroid hormone, iron supplement; any appropriate treatment described elsewhere herein; any treatment described herein for use when a high proportion of SNPs listed in Table 13-34 is detected in the biological sample; a lifestyle modification (e.g. to diet, smoking, alcohol consumption, weight, physical activity); surgery, e.g., thyroidectomy; physical therapy; speech therapy, psychological counseling; pain management therapy; and increased disease monitoring (e.g., labs including thyroid hormone and antibody tests, thyroid imaging, thyroid biopsy).

In some embodiments, the NP cluster is associated with angioedema, and the treatment is selected from one or more treatment for angioedema or a condition secondary to angioedema known to those of skill in the art, including: any approved or experimental drug to treat or prevent angioedema, e.g., epinephrine, antihistamines, corticosteroids, C1 inhibitor; any appropriate treatment described elsewhere herein; any treatment described herein for use when a high proportion of SNPs listed in Table 13-5 or 13-48 is detected in the biological sample; a lifestyle modification (e.g. to avoid trigger, e.g., allergens, drugs, infection, trauma); physical therapy; speech therapy, psychological counseling; pain management therapy; and increased disease monitoring (e.g., allergy testing).

In some embodiments, the NP cluster is associated with Behcet syndrome, and the treatment is selected from one or more treatment for Behcet syndrome or a condition secondary to Behcet syndrome known to those of skill in the art, including: any approved or experimental drug to treat or prevent Behcet syndrome, e.g., corticosteroid, local anesthetic, NSAID, colchicine, sulfasalazine, azathioprine, anticoagulant, methotrexate, cyclosporine, cyclophosphamide, chlorambucil, immunosuppressant, interferon alpha, anti-TNF inhibitor (e.g., infliximab, adalimumab), apremilast; any appropriate treatment described elsewhere herein, including treatments for use when a high proportion of SNPs listed in Table 13-8 is detected in the biological sample; a lifestyle modification (e.g., to physical activity, sleep, stress level); pain management therapy; and increased disease monitoring (e.g., for infection when on immunosuppressant).

In some embodiments, the NP cluster is associated with chronic inflammatory disease, and the treatment is selected from one or more treatment for chronic inflammatory disease (including but not limited to asthma, COPD, inflammatory bowel disease, Crohn's, ulcerative colitis, rheumatoid arthritis, SLE, psoriasis, psoriatic arthritis, ankylosing spondylitis, juvenile idiopathic arthritis), or a condition secondary to chronic inflammatory disease known to those of skill in the art, including: any approved or experimental drug known to those of skill in the art for treating or preventing chronic inflammatory disease, e.g., an immunomodulator, immune suppressing small molecule drug, biologic, DMARD, NSAID, corticosteroid (including oral, topical, injectable), vitamin D; any appropriate treatment described elsewhere herein, including as described herein for SLE, Sjogren's syndrome, inflammatory bowel disease, ulcerative colitis, and Type 1 diabetes; any treatment described herein for use when a high proportion of SNPs listed in Table 13-8, 13-18, 13-23, 13-5, 13-14, 13-33, 13-34, or 13-36 is detected in the biological sample; any lifestyle modification known to those of skill in the art and described in the literature (e.g. to diet, smoking, stress, physical activity, sun exposure, exposure to infection); any surgical intervention known to those of skill in the art and described in the literature, e.g., arthroscopy, joint replacement; physical therapy; psychological counseling; pain management therapy; and increased disease monitoring of any feature of the disease or disorder known to those of skill in the art and described in the literature (e.g., clinical examination, labs including inflammatory markers, blood count and autoantibody tests, joint imaging, kidney function, heart function, nerve conductance).

A disease or disorder may be associated with an NP cluster as disclosed herein, e.g., in Table 13. A selected treatment can target a disease or disorder associated with any one or more cluster as set forth in a Table herein selected from: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67.

In some embodiments, the NP cluster is that set forth in Table 13-3, the associated disease or disorder is myocardial injury, and the selected treatment is any known to those of skill in the art, e.g., as set forth herein. In some embodiments, the NP cluster is that set forth in Table 13-5, the associated disease or disorder is SLE, and the selected treatment is any known to those of skill in the art, e.g., as set forth herein. In some embodiments, the NP cluster is that set forth in Table 13-5, the associated disease or disorder is ischemic stroke, and the selected treatment is any known to those of skill in the art, e.g., as set forth herein. In some embodiments, the NP cluster is that set forth in Table 13-5, the associated disease or disorder is coronary artery disease, and the selected treatment is any known to those of skill in the art, e.g., as set forth herein. In some embodiments, the NP cluster is that set forth in Table 13-13, the associated disease or disorder is X-linked thrombocytopenia, and the selected treatment is any known to those of skill in the art, e.g., as set forth herein. In some embodiments, the NP cluster is that set forth in Table 13-33, the associated disease or disorder is cardiomyopathy, and the selected treatment is any known to those of skill in the art, e.g., as set forth herein. In some embodiments, the NP cluster is that set forth in Table 13-33, the associated disease or disorder is diabetes, and the selected treatment is any known to those of skill in the art, e.g., as set forth herein. In some embodiments, the NP cluster is that set forth in Table 13-12, the associated disease or disorder is atherosclerosis, and the selected treatment is any known to those of skill in the art, e.g., as set forth herein. In some embodiments, the NP cluster is that set forth in Table 13-14, the associated disease or disorder is SLE and the selected treatment is any known to those of skill in the art, e.g., as set forth herein. In some embodiments, the NP cluster is set forth in Table 13-14, the associated disease or disorder is dyslipidemia, and the selected treatment is any known to those of skill in the art, e.g., as set forth herein. In some embodiments, the NP cluster is that set forth in Table 13-18, the associated disease or disorder is dyslipidemia, and the selected treatment is any known to those of skill in the art, e.g., as set forth herein. In some embodiments, the NP cluster is set forth in Table 13-18, the associated disease or disorder is ulcerative colitis, and the selected treatment is any known to those of skill in the art, e.g., as set forth herein. In some embodiments, the NP cluster is set forth in Table 13-18, the associated disease or disorder is inflammatory bowel disease, and the selected treatment is any known to those of skill in the art, e.g., as set forth herein. In some embodiments, the NP cluster is set forth in Table 13-23, the associated disease or disorder is ischemic heart disease, and the selected treatment is any known to those of skill in the art, e.g., as set forth herein. In some embodiments, the NP cluster is set forth in Table 13-23, the associated disease or disorder is atherosclerosis, and the selected treatment is any known to those of skill in the art, e.g., as set forth herein. In some embodiments, the NP cluster is set forth in Table 13-23, the associated disease or disorder is Sjogren's syndrome, and the selected treatment is any known to those of skill in the art, e.g., as set forth herein. In some embodiments, the NP cluster is set forth in Table 13-28, the associated disease or disorder is SLE, and the selected treatment is any known to those of skill in the art, e.g., as set forth herein. In some embodiments, the NP cluster is set forth in Table 13-28, the associated disease or disorder is cardiovascular disease, and the selected treatment is any known to those of skill in the art, e.g., as set forth herein. In some embodiments, the NP cluster is set forth in Table 13-28, the associated disease or disorder is hemolytic anemia, and the selected treatment is any known to those of skill in the art, e.g., as set forth herein. In some embodiments, the NP cluster is set forth in Table 13-34, the associated disease or disorder is obesity, and the selected treatment is any known to those of skill in the art, e.g., as set forth herein. In some embodiments, the NP cluster is set forth in Table 13-34, the associated disease or disorder is diabetes type 1, and the selected treatment is any known to those of skill in the art, e.g., as set forth herein. In some embodiments, the NP cluster is set forth in Table 13-34, the associated disease or disorder is thyroid disease, and the selected treatment is any known to those of skill in the art, e.g., as set forth herein. In some embodiments, the NP cluster is set forth in Table 13-48, the associated disease or disorder is atherosclerosis, and the selected treatment is any known to those of skill in the art, e.g., as set forth herein. In some embodiments, the NP cluster is set forth in Table 13-48, the associated disease or disorder is angioedema, and the selected treatment is any known to those of skill in the art, e.g., as set forth herein. In some embodiments, the NP cluster is set forth in Table 13-36, the associated disease or disorder is diabetes type 2, and the selected treatment is any known to those of skill in the art, e.g., as set forth herein. In some embodiments, the NP cluster is set forth in Table 13-8, the associated disease or disorder is Behcet syndrome, and the selected treatment is any known to those of skill in the art, e.g., as set forth herein. In some embodiments, the NP cluster is set forth in Table 13-8, the associated disease or disorder is chronic inflammatory disease, and the selected treatment is any known to those of skill in the art, e.g., as set forth herein.

In certain embodiments, the method comprises administering the treatment to the patient, based on the CAD state of the patient. In certain embodiments, the treatment is selected, recommended, and/or administered based on i) the presence of the one or more SNPs in the biological sample from the patient, and/or ii) the patient having one or more symptoms of CAD. In certain embodiments, the treatment is administered based on i) the presence of the one or more SNPs in the biological sample from the patient, and/or ii) the patient having one or more symptoms of CAD, and the method can be directed to treating CAD. In certain embodiments, the treatment is administered based on i) the presence of the one or more SNPs in the biological sample from the patient, and ii) the patient having one or more symptoms of CAD, and the method can be directed to treating CAD. The treatment selected, recommended, and/or administered can be based on the one or more SNPs detected (e.g., present) in the biological sample. In certain embodiments, the treatment administered is based on the one or more SNPs detected (e.g., present) in the biological sample.

In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and/or ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and/or ii) the patient has one or more symptoms of CAD. In certain embodiments, the treatment is administered i) when one or more SNPs selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample and ii) the patient has one or more symptoms of CAD. In certain embodiments, the treatment is administered when i) a high proportion of SNPs listed in at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, is present in the biological sample, and ii) the patient has one or more symptoms of CAD.

The treatment selected, recommended, and/or administered can be based on the SNPs present in the biological sample. The treatment administered can be based on the SNPs present in the biological sample. In certain embodiments, the treatment can be based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66; wherein one or more SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein one or more SNPs listed in the Table are present in the biological sample. In certain embodiments, the treatment is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66; wherein a high proportion of SNPs listed in the at least one Table, is present in the biological sample. In certain embodiments, the treatment targets at least one or more genes listed in a Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; and 13-66, wherein a high proportion of SNPs listed in the Table are present in the biological sample. In certain embodiments, the treatment can be based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein one or more SNPs listed in the at least one Table, is present in the biological sample. In certain embodiments, the treatment targets at least one or more genes listed in a Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein one or more SNPs listed in the Table are present in the biological sample. In certain embodiments, the treatment is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein a high proportion of SNPs listed in the at least one Table, is present in the biological sample. In certain embodiments, the treatment targets at least one or more genes listed in a Table selected from the Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein a high proportion of SNPs listed in the Table are present in the biological sample. A high proportion of SNPs listed in a Table present in the biological sample can refer at least 20%, 30%, 40%, 50%, 60%, 70%, 80%, 90%, 95%, or 100% of the SNPs listed in the Table are present in the biological sample.

Treatments based on a functional annotation of a respective Table may target, i) one or more biological pathways (see for example, Table 13) associated with the respective Table, ii) one or more genes listed in the respective Table and/or iii) genes and/or biological pathways upstream of or related to the biological pathways associated with, gene listed in and/or SNPs listed in the respective Table. The treatment can include one or more treatments of CAD. In certain embodiments, the treatment is configured to treat CAD. In certain embodiments, the treatment is configured to reduce severity of CAD. In certain embodiments, the treatment is configured to reduce a risk of developing CAD. In certain embodiments, the treatment comprises a treatment for atherosclerosis. In certain embodiments, the treatment comprises an anti-IFN antibody such as anifrolumab; an anti-oxidized LDL antibody such as orticumab, an anti-PCSK9 such as alirocumab and/or evolocumab; a JAK inhibitor such as baricitinib and/or tofacitinib; a MTOR inhibitor rapamycin; a MPO inhibitor such as PF-1355; an ACE inhibitor such as captopril; a statin; or any combination thereof. In certain embodiments, the treatment comprises a pharmaceutical composition.

In certain embodiments, the patient is determined to be at risk of developing CAD, when the one or more SNPs detected are present in the biological sample, but the patient does not have one or more symptoms of CAD. In certain embodiments, the patients is recommended, performed with and/or administered one or more lifestyle changes, when the one or more SNPs detected are present in the biological sample, but the patient does not have one or more symptoms of CAD. The one or more lifestyle changes can include monitoring, such as frequent monitoring the patient for one or more symptoms of CAD. Monitoring can include monitoring through echocardiogram, exercise stress test, chest X-ray, cardiac catheterization, etc, for one or more symptoms of CAD. Frequent monitoring can include a monitoring at a higher frequency, compared to past (e.g., past 1 month, 3 months, 6 months, 1 year, 2 years, 3 years, 5 years, 10 years, etc.) monitoring of the patient.

In certain embodiments, one or more SNPs selected from the SNPs listed in Table 13-5, are present in the biological sample, and the treatment comprises a pharmaceutical composition for anti-platelet/coagulant therapy such as warfarin, and/or aspirin; targeting oxidized LDL molecules such as orticumab; targeting PCSK9 such as alirocumab and/or evolocumab; targeting IKZF1/IKZF3 such as iberdimide; or any combination thereof. In certain embodiments, one or more SNPs selected from the SNPs listed in Table 13-5, are present in the biological sample, and the treatment comprises warfarin, aspirin, orticumab, alirocumab, evolocumab, iberdimide; or any combination thereof. In certain embodiments, one or more SNPs selected from the SNPs listed in Table 13-8, are present in the biological sample, and the treatment comprises a pharmaceutical composition targeting TYK2 such as BMS-986165; targeting IL12RB1/2 such as ustekinumab; comprising an anti-interferon antibody such as anifrolumab; comprising a MTOR inhibitor such as rapamycin; or any combination thereof. In certain embodiments, one or more SNPs selected from the SNPs listed in Table 13-8, are present in the biological sample, and the treatment comprises BMS-986165, ustekinumab, anifrolumab, rapamycin, or any combination thereof. In certain embodiments, one or more SNPs selected from the SNPs listed in Table 13-2, are present in the biological sample, and the treatment comprises a pharmaceutical composition targeting CD38 such as daratumumab; targeting TNFSF13 such as belimumab; targeting SLAMF7 such as elotuzumab; targeting CD80/86 such abatacept; targeting ITGA4 such as natalizumab; a MPO inhibitor such as PF-1355; or any combination thereof. In certain embodiments, one or more SNPs selected from the SNPs listed in Table 13-2, are present in the biological sample, and the treatment comprises a pharmaceutical composition comprises daratumumab, belimumab, elotuzumab, abatacept, natalizumab, PF-1355, colchicine or any combination thereof. In certain embodiments, one or more SNPs selected from the SNPs listed in Table 13-3, are present in the biological sample, and the treatment comprises a pharmaceutical composition comprising a JAK inhibitor such as baricitinib and/or tofacitinib; targeting IL6R such as sarliumab; targeting IL21 such as BOS-161721; or any combination thereof. In certain embodiments, one or more SNPs selected from the SNPs listed in Table 13-3, are present in the biological sample, and the treatment comprises baricitinib, tofacitinib, sarliumab, BOS-161721, or any combination thereof. In certain embodiments, one or more SNPs selected from the SNPs listed in Table 13-4, are present in the biological sample, and the treatment comprises a pharmaceutical composition comprising a MTOR inhibitor such as rapamycin and/or atorvastatin. In certain embodiments, one or more SNPs selected from the SNPs listed in Table 13-4, are present in the biological sample, and the treatment comprises rapamycin; and/or atorvastatin. In certain embodiments, one or more SNPs selected from the SNPs listed in Table 13-44, are present in the biological sample, and the treatment comprises a pharmaceutical composition comprising a cholesterol inhibitor such as pravastatin; and/or ezetimibe. In certain embodiments, one or more SNPs selected from the SNPs listed in Table 13-44, are present in the biological sample, and the treatment comprises pravastatin and/or ezetimibe. In certain embodiments, one or more SNPs selected from the SNPs listed in Table 13-33, are present in the biological sample, and the treatment comprises a pharmaceutical composition for treating irregular heart beat such as amiodarone; comprising a Ca+2 blocker such as nifepidine; a ACE inhibitor such as captopril; or any combination thereof. In certain embodiments, one or more SNPs selected from the SNPs listed in Table 13-33, are present in the biological sample, and the treatment=comprises amiodarone, nifepidine, captopril, or any combination thereof. In certain embodiments, one or more SNPs selected from the SNPs listed in Table 13-19, are present in the biological sample, and the treatment comprises a pharmaceutical composition a PDE4 inhibitor such as apremilast; a PDE5 inhibitor such as dipyridamole; a Ca+2 blocker such as nifepidine; or any combination thereof. In certain embodiments, one or more SNPs selected from the SNPs listed in Table 13-19, are present in the biological sample, and the treatment comprises apremilast, dipyridamole, nifepidine, or any combination thereof.

In certain embodiments, a high proportion of SNPs listed in Table 13-5, are present in the biological sample, and the treatment comprises a pharmaceutical composition for anti-platelet/coagulant therapy such as warfarin, and/or aspirin; targeting oxidized LDL molecules such as orticumab; targeting PCSK9 such as alirocumab and/or evolocumab; targeting IKZF1/IKZF3 such as iberdimide; or any combination thereof. In certain embodiments, a high proportion of SNPs listed in Table 13-5, are present in the biological sample, and the treatment comprises warfarin, aspirin, orticumab, alirocumab, evolocumab, iberdimide; or any combination thereof. In certain embodiments, a high proportion of SNPs listed in Table 13-8, are present in the biological sample, and the treatment comprises a pharmaceutical composition targeting TYK2 such as BMS-986165; targeting IL12RB1/2 such as ustekinumab; comprising an anti-interferon antibody such as anifrolumab; comprising a MTOR inhibitor such as rapamycin; or any combination thereof. In certain embodiments, a high proportion of SNPs listed in Table 13-8, are present in the biological sample, and the treatment comprises BMS-986165, ustekinumab, anifrolumab, rapamycin, or any combination thereof. In certain embodiments, a high proportion of SNPs listed in Table 13-2, are present in the biological sample, and the treatment comprises a pharmaceutical composition targeting CD38 such as daratumumab; targeting TNFSF13 such as belimumab; targeting SLAMF7 such as elotuzumab; targeting CD80/86 such abatacept; targeting ITGA4 such as natalizumab; a MPO inhibitor such as PF-1355; or any combination thereof. In certain embodiments, a high proportion of SNPs listed in Table 13-2, are present in the biological sample, and the treatment comprises a pharmaceutical composition comprises daratumumab, belimumab, elotuzumab, abatacept, natalizumab, PF-1355, colchicine or any combination thereof. In certain embodiments, a high proportion of SNPs listed in Table 13-3, are present in the biological sample, and the treatment comprises a pharmaceutical composition comprising a JAK inhibitor such as baricitinib and/or tofacitinib; targeting IL6R such as sarliumab; targeting IL21 such as BOS-161721; or any combination thereof. In certain embodiments, a high proportion of SNPs listed in Table 13-3, are present in the biological sample, and the treatment comprises baricitinib, tofacitinib, sarliumab, BOS-161721, or any combination thereof. In certain embodiments, a high proportion of SNPs listed in Table 13-4, are present in the biological sample, and the treatment comprises a pharmaceutical composition comprising a MTOR inhibitor such as rapamycin and/or atorvastatin. In certain embodiments, a high proportion of SNPs listed in Table 13-4, are present in the biological sample, and the treatment comprises rapamycin; and/or atorvastatin. In certain embodiments, a high proportion of SNPs listed in Table 13-44, are present in the biological sample, and the treatment comprises a pharmaceutical composition comprising a cholesterol inhibitor such as pravastatin; and/or ezetimibe. In certain embodiments, a high proportion of SNPs listed in Table 13-44, are present in the biological sample, and the treatment comprises pravastatin and/or ezetimibe. In certain embodiments, a high proportion of SNPs listed in Table 13-33, are present in the biological sample, and the treatment comprises a pharmaceutical composition for treating irregular heart beat such as amiodarone; comprising a Ca+2 blocker such as nifepidine; a ACE inhibitor such as captopril; or any combination thereof. In certain embodiments, a high proportion of SNPs listed in Table 13-33, are present in the biological sample, and the treatment comprises amiodarone, nifepidine, captopril, or any combination thereof. In certain embodiments, a high proportion of SNPs listed in Table 13-19, are present in the biological sample, and the treatment comprises a pharmaceutical composition a PDE4 inhibitor such as apremilast; a PDE5 inhibitor such as dipyridamole; a Ca+2 blocker such as nifepidine; or any combination thereof. In certain embodiments, a high proportion of SNPs listed in Table 13-19, are present in the biological sample, and the treatment comprises apremilast, dipyridamole, nifepidine, or any combination thereof.

An aspect of the present disclosure is directed to a method for determining myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders state in a patient. The method can include (i) determining one or more SNPs selected from SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; in a biological sample from the patient; and determining myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders state of the patient, based on the presence of the one or more SNPs in the biological sample.

Determining myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders state of the patient can include, determining whether the patient has myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders respectively, and/or whether the patient is at risk of developing myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders respectively

In certain embodiments, the one or more SNPs include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 SNPs. In certain embodiments, the one or more SNPs comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451, or any value or range there between, SNPs. In certain embodiments, the one or more SNPs consist of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 or any value or range there between, SNPs. In certain embodiments, the one or more SNPs include independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of one or more Tables selected from Tables: 13-2; 13-3; 13-4; 13-5; 13-8; 13-9; 13-12; 13-13; 13-15; 13-16; 13-18; 13-19; 13-21; 13-23; 13-28; 13-32; 13-33; 13-34; 13-36; 13-43; 13-44; 13-51; 13-55; 13-60; 13-65; 13-67; 13-1; 13-10; 13-24; 13-25; 13-30; 13-40; 13-45; 13-50; 13-56; 13-61; and 13-64. In certain embodiments, the one or more SNPs include independently at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, or all, or any range or value there between SNPs from each of one or more Tables selected from Tables: 13-2; 13-3; 13-4; 13-5; 13-8; 13-9; 13-12; 13-13; 13-15; 13-16; 13-18; 13-19; 13-21; 13-23; 13-28; 13-32; 13-33; 13-34; 13-36; 13-43; 13-44; 13-51; 13-55; 13-60; 13-65; and 13-67.

In certain embodiments, a disease risk score for the patient is calculated based on presence of the one or more SNPs in the biological sample, and the myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders state of the patient is determined based on the disease risk score. The one or more SNPs in the biological sample can be determined by analyzing nucleic acid of the patient in the biological sample. The nucleic acid can be DNA and/or RNA. Analyzing the nucleic acid of the patient, can include analyzing RNA and/or DNA of the patient, in the biological sample. In certain embodiments, analyzing the RNA can include, analyzing mRNA. In certain embodiments, analyzing the nucleic acid includes RNA sequencing. In certain embodiments, analyzing the nucleic acid includes mRNA sequencing. In certain embodiments, analyzing the nucleic acid includes DNA sequencing. In certain embodiments, analyzing the nucleic acid measuring expression of the genes associated with the one or more SNPs. The genes associated with a SNPs, can include the E-, C-, T, and/or P-gene associated with the SNP. In certain embodiments, analyzing the nucleic acid includes performing enrichment analysis of the genes associated with the one or more SNPs. The enrichment analysis can be performed using gene set variation analysis (GSVA), gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof. In certain embodiments, the enrichment analysis is performed using GSVA. In certain embodiments, the enrichment analysis is performed using GSVA, wherein at least one GSVA score is generated for each of one or more Tables selected from Table: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; wherein for a respective Table the at least one GSVA score is generated for enrichment of at least gene within a gene cluster mapped to the SNP cluster of the respective Table. The mapping can be performed using the methods of step (c), (d), and/or (e), as described above.

The biological sample can be a blood sample, isolated peripheral blood mononuclear cells (PBMCs), tissue biopsy sample, nasal fluid, saliva, urine, stool, or any derivative thereof. In certain embodiments, the biological sample can be a blood sample or any derivative thereof. In certain embodiments, the biological sample can be PBMCs or any derivative thereof. In certain embodiment, the patient has lupus. In certain embodiments, the patient does not have lupus. In certain embodiments, the patient is at an elevated risk of having lupus. In certain embodiments, the patient is asymptomatic for lupus.

In certain embodiments, the method comprises administering a treatment to the patient based, on the myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders state of the patient. The treatment can be for myocardial infarction, ischemic stroke, coronary atherosclerosis, cardiomyopathy, glomerulonephritis, depression, inflammatory bowel disease, asthma, chronic obstructive pulmonary disease (COPD), diabetes mellitus, nonalcoholic fatty liver disease, and/or metabolic disorders. In certain embodiment, the treatment comprises a pharmaceutical composition.

In certain embodiments, the method is directed to determining myocardial infarction state of the patient. In certain embodiments, the method is directed to determining ischemic stroke state of the patient. In certain embodiments, the method is directed to determining coronary atherosclerosis state of the patient. In certain embodiments, the method is directed to determining cardiomyopathy state of the patient. In certain embodiments, the method is directed to determining glomerulonephritis state of the patient. In certain embodiments, the method is directed to determining depression state of the patient. In certain embodiments, the method is directed to determining inflammatory bowel disease state of the patient. In certain embodiments, the method is directed to determining asthma state of the patient. In certain embodiments, the method is directed to determining COPD state of the patient. In certain embodiments, the method is directed to determining diabetes mellitus state of the patient. In certain embodiments, the method is directed to determining nonalcoholic fatty liver disease state of the patient. In certain embodiments, the method is directed to determining metabolic disorders state of the patient.

In certain embodiments, the treatment administered is for myocardial infarction, and is based on the determination that the patient has myocardial infarction, and/or the patient is at risk of developing myocardial infarction, wherein the treatment is configured to treat, reduce severity of, and/or reduce risk of developing, myocardial infarction. In certain embodiments, the treatment administered is for ischemic stroke, and is based on the determination that the patient has ischemic stroke, and/or the patient is at risk of developing ischemic stroke, wherein the treatment is configured to treat, reduce severity of, and/or reduce risk of developing, ischemic stroke. In certain embodiments, the treatment administered is for coronary atherosclerosis, and is based on the determination that the patient has coronary atherosclerosis, and/or the patient is at risk of developing coronary atherosclerosis, wherein the treatment is configured to treat, reduce severity of, and/or reduce risk of developing, coronary atherosclerosis. In certain embodiments, the treatment administered is for cardiomyopathy, and is based on the determination that the patient has cardiomyopathy, and/or the patient is at risk of developing cardiomyopathy, wherein the treatment is configured to treat, reduce severity of, and/or reduce risk of developing, cardiomyopathy. In certain embodiments, the treatment administered is for glomerulonephritis, and is based on the determination that the patient has glomerulonephritis, and/or the patient is at risk of developing glomerulonephritis, wherein the treatment is configured to treat, reduce severity of, and/or reduce risk of developing, glomerulonephritis. In certain embodiments, the treatment administered is for depression, and is based on the determination that the patient has depression, and/or the patient is at risk of developing depression, wherein the treatment is configured to treat, reduce severity of, and/or reduce risk of developing, depression. In certain embodiments, the treatment administered is for inflammatory bowel disease, and is based on the determination that the patient has inflammatory bowel disease, and/or the patient is at risk of developing inflammatory bowel disease, wherein the treatment is configured to treat, reduce severity of, and/or reduce risk of developing, inflammatory bowel disease. In certain embodiments, the treatment administered is for asthma, and is based on the determination that the patient has asthma, and/or the patient is at risk of developing asthma, wherein the treatment is configured to treat, reduce severity of, and/or reduce risk of developing, asthma. In certain embodiments, the treatment administered is for COPD, and is based on the determination that the patient has COPD, and/or the patient is at risk of developing COPD, wherein the treatment is configured to treat, reduce severity of, and/or reduce risk of developing, COPD. In certain embodiments, the treatment administered is for diabetes mellitus, and is based on the determination that the patient has diabetes mellitus, and/or the patient is at risk of developing diabetes mellitus, wherein the treatment is configured to treat, reduce severity of, and/or reduce risk of developing, diabetes mellitus. In certain embodiments, the treatment administered is for nonalcoholic fatty liver disease, and is based on the determination that the patient has nonalcoholic fatty liver disease, and/or the patient is at risk of developing nonalcoholic fatty liver disease, wherein the treatment is configured to treat, reduce severity of, and/or reduce risk of developing, nonalcoholic fatty liver disease. In certain embodiments, the treatment administered is for metabolic disorders, and is based on the determination that the patient has metabolic disorders, and/or the patient is at risk of developing metabolic disorders, wherein the treatment is configured to treat, reduce severity of, and/or reduce risk of developing, metabolic disorders.

In certain embodiments, the treatment administered can be selected, based on functional annotation of at least one Table selected from Table: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; wherein at least one SNP present in the biological sample, is listed in the at least one Table. The patient can be a human patient.

An aspect of the present disclosure is directed to use of the shared NPs such as SNPs, and/or genes disclosed above and elsewhere herein.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity©.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash© Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowser, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Biological Data Analysis

Certain embodiments, of the present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method comprises identifying a disease or disorder of the subject at a sensitivity or specificity of at least about 70%. In some embodiments, the method comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained. In some embodiments, not more than 10 mL, 5 mL, 1 mL, 0.5 mL, or 250, 200, 150, 100, 50, 40, 30, 20, 10, 9, 8, 7, 6, 5, 4, 3, 2, or 1 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness.

For example, a method as described herein may be performed on a subject prior to, and after, treatment with a first, second, and/or third disease condition therapy to measure the disease's progression or regression in response to the first, second, and/or third disease condition therapy. The first, second, and/or third disease can be as described above.

After obtaining a sample from the subject, the sample may be processed to generate datasets indicative of a disease or disorder of the subject. For example, a presence, absence, or quantitative assessment of nucleic acid molecules of the sample from a panel of condition-associated genomic loci or nucleotide polymorphism may be indicative of first, second, and/or third disease condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that may also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, SIP1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that may be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in first, second and/or third disease patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

### BIG-C™ Big Data Analysis Tool

BIG-C® is a fast and efficient cloud-based tool to functionally categorize gene products. With coverage of over 80% of the genome, BIG-C® leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-C® may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-C® categories are cross-examined with the GO and KEGG terms to obtain additional information and insights.

A sample BIG-C® workflow may comprise the following steps. First, SLE genomic datasets are derived from whole blood, peripheral blood mononuclear cells, affected tissues, and purified immune cells. Second, datasets are analyzed using DE analysis (as shown by a differential expression heatmap) or Weighted Gene Coexpression Network Analysis (WGCNA) (as shown by a gene coexpression plot). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-C® is leveraged to separate the individual annotated genes into one of 53 functional categories (e.g., as described by Labonte et al. 2018, “Identification of alterations in macrophage activation associated with disease activity in systemic lupus erythematosus,” PloS one, 13(12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ may be used downstream of the BIG-C® (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. When combined with BIG-C® categories, the cellular activity may be correlated to specific functions within a given cell type.

A sample I-Scope™ workflow may comprise the following steps. First, candidate genes are identified from SLE (systemic lupus erythematosus) datasets potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ may be used downstream of the BIG-C® (Biologically Informed Gene-Clustering) tool to understand which tissue cell types are present. In conjunction with I-Scope™ (which provides information related to immune cells), T-Scope™ may be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories, ultimately allowing for cellular activity analysis across multiple samples and disease states. When combined with BIG-C® categories, the cellular activity may be correlated to specific functions within a given tissue cell type.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

### CellScan Big Data Analysis Tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide undiscovered insights.

CellScan™ may go beyond typical ‘omics analysis by performing one or more of the following: functionally categorizing genes and their products (e.g., using BIG-C®); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. An example list of reference databases for the content in CellScan™, with both human and mouse species-specific identifiers supported.

### MS (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPA®, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between −100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 1 may be performed of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25(10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between −1 and +1 indicating levels of down-regulation and up-regulation respectively.

A sample MS-Scoring™ 2 workflow may comprise the following steps. First, a signaling pathway of interest is selected from the MS-Scoring™ 2 menu Second, a raw gene expression data is inputted into the MS-Scoring™ 2 tool. Third, enrichment of signaling pathway(s) is assessed on a patient by patient basis. Fourth, the data may then be used to drive insight for the target signaling pathways in individual patient samples.

Results from GSVA Analysis on SLE (systemic lupus erythematosus) signaling pathways may be, e.g., as described by Hänzelmann et al., “GSVA: Gene Set Variation Analysis for Microarray and RNA-Seq Data,” BMC Bioinformatics, vol. 14, no. 1, 2013, p. 7., which is incorporated herein by reference in its entirety.

### CoLTs® (Combined Lupus Treatment Scoring) Analysis Tool

A scoring method called CoLTs®, or Combined Lupus Treatment Scoring, may be configured to assessing and prioritizing the repositioning potential of drug therapies. CoLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available.

CoLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs®' prediction of Stelara/Ustekinumab to be a top priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155, 2018, pp. 1330-1339, which is incorporated herein by reference in its entirety). CoLTs® may be calibrated on SoC (Standard of Care) therapies for the individual autoimmune disease being assessed.

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25(10), 1150-1170, which is incorporated herein by reference in its entirety). For example, CoLT Scoring® of SOC Therapies in Lupus (Belimumab, HCQ, and Rituximab) may be performed.

### Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

Target-Scoring™ may be configured to assessing and prioritizing the potential of molecular targets for further development of drug therapies. The Target-Scoring™ tool is very similar to CoLTs® except it approaches the need for new SLE therapies from a different angle. Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers may better prioritize the development of novel drug therapies addressing the assessed targets of interest.

Target-Scoring™ may utilize 19 different scoring categories to derive a composite score that ranges from −13 to +27 for the suitability of a gene target for SLE therapy development. Target-Scoring™ may be validated through repeated scoring of potential therapies as well as by clinicians (e.g., clinicians specializing in the field of immunology).

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module may comprise computer systems that connect laboratory hardware or instrumentation with computer systems that process laboratory data. In one embodiment, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as first, second, and/or third disease condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as first, second, and/or third disease condition) and individuals not having the condition (e.g., healthy individuals, or individuals who do not have first, second, and/or third disease condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as first, second, and/or third disease condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as first, second, and/or third disease condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as first, second, and/or third disease condition), a prognosis of one or more conditions (e.g., a disease or disorder, such as first, second, and/or third disease condition), a risk of having one or more conditions (e.g., a disease or disorder, such as first, second, and/or third disease condition), a treatment history of one or more conditions (e.g., a disease or disorder, such as first, second, and/or third disease condition), a history of previous treatment for one or more conditions (e.g., a disease or disorder, such as first, second, and/or third disease condition), a history of prescribed medications, a history of prescribed medical devices, age, height, weight, sex, smoking status, and one or more symptoms of the subject.

For example, the disease or disorder may comprise one or more of: lupus, coronary artery disease (CAD), myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis. As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as first, second, and/or third disease condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1},{positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as first, second, and/or third disease condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as first, second, and/or third disease condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as first, second, and/or third disease condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as first, second, and/or third disease condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as first, second, and/or third disease condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as first, second, and/or third disease condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}{2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as first, second, and/or third disease condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as first, second, and/or third disease condition). In some embodiments, the sample is independent of samples used to train the trained algorithm.

The trained algorithm may be trained with a first number of independent training samples associated with a presence of the condition (e.g., a disease or disorder, such as first, second, and/or third disease condition) and a second number of independent training samples associated with an absence of the condition (e.g., a disease or disorder, such as first, second, and/or third disease condition). The first number of independent training samples associated with presence of the condition (e.g., a disease or disorder, such as first, second, and/or third disease condition) may be no more than the second number of independent training samples associated with absence of the condition (e.g., a disease or disorder, such as first, second, and/or third disease condition). The first number of independent training samples associated with a presence of the condition (e.g., a disease or disorder) may be equal to the second number of independent training samples associated with an absence of the condition (e.g., a disease or disorder, such as first, second, and/or third disease condition). The first number of independent training samples associated with a presence of the condition (e.g., a disease or disorder, such as first, second, and/or third disease condition) may be greater than the second number of independent training samples associated with an absence of the condition (e.g., a disease or disorder, such as first, second, and/or third disease condition).

The trained algorithm may comprise a classifier configured to identify the presence (e.g., positive test result) or absence (e.g., negative test result) of one or more conditions (e.g., a disease or disorder, such as first, second, and/or third disease condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as first, second, and/or third disease condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as first, second, and/or third disease condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as first, second, and/or third disease condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as first, second, and/or third disease condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as first, second, and/or third disease condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as first, second, and/or third disease condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples.

### Kits

The present disclosure provides kits for identifying or monitoring a disease or disorder (e.g., first, second, and/or third disease condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., first, second, and/or third disease condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., first, second, and/or third disease condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

### EXAMPLES

The following illustrative examples are representative of embodiments of the software applications, systems, and methods described herein and are not meant to be limiting in any way.

### Example 1: Mendelian Randomization and Molecular Network Analysis Demonstrate Positive and Negative Genetic Causal Associations Between Systemic Lupus Erythematosus and Coronary Artery Disease

Coronary artery disease (CAD) is a leading cause of death in patients with systemic lupus erythematosus (SLE). Despite clinical evidence supporting an association between SLE and CAD, pleiotropy-adjusted genetic association studies have been limited and focused on only a few common risk loci. Here, we report a Mendelian Randomization (MR) analysis that has identified large sets of SLE-risk polymorphisms with positive and negative implications on CAD. We mapped associated SNPs to genes and applied unsupervised clustering based on protein-protein interactions (PPI) to identify biological networks comprised of positive and negative causal sets of genes. In addition to identifying a net positive causal estimate of SLE-associated non-HLA SNPs on CAD by traditional MR approaches, we confirmed the casual effects of specific SNP-to-gene modules on CAD using only SNPs mapping to each PPI-defined functional gene set as instrumental variables. This PPI-based MR approach elucidated various molecular pathways with causal implications between SLE and CAD. These results have defined a broad range of SLE-risk polymorphisms contributing to CAD and have identified biologic pathways likely involved in both pathologies and facilitated drug predictions to reveal known and novel therapeutic interventions for managing the unique inflammatory environment contributing to CAD in SLE.

Systemic lupus erythematosus (SLE) is a female predominant, autoimmune disease characterized by immune dysregulation and multi-organ inflammation that is frequently associated with the development of cardiovascular disease (CVD) (1, 2). SLE exhibits hyperactivity of the innate and adaptive immune systems, increased production of numerous autoantibodies, and disturbed cytokine balance (3). Although CVD is not a diagnostic criterion of SLE and was not included in the original descriptions of the disease, it is currently the main cause of death in SLE (4-6) with coronary artery disease (CAD) directly responsible for one-third to one-half of all CVD cases (7, 8) and 30% of deaths (9). Notably, whereas mortality from infections and active disease have decreased in SLE patients, CVD-related death rates have not improved (10) and the standardized mortality ratio related to CVD has actually increased (11). Women with SLE have a significantly increased risk of stroke and myocardial infarction along with elevated incidence of asymptomatic atherosclerosis compared to the general population (12, 13). Furthermore, traditional CVD risk factors, such as cholesterol, blood pressure, and smoking status fail to fully account for the overall higher risk of acute CVD events in SLE, although the underlying mechanisms remain unknown (14-17). This lack of an understanding for the increased risk of CVD in SLE has resulted in limited treatment options and the puzzling juxtaposition that despite the efficacy of statins and ACEIs/ARBs in treating the general population, they appear to have little effect on CVD outcomes in SLE patients (5, 18). As a result, even though SLE has a prevalence of only about 70 per 100,000, it ranks among the leading causes of death in young women (1), despite the omission of lupus diagnoses in almost half of SLE patients' death certificates (19, 20).

Genetic predisposition imposes important risk factors for both SLE and CVD (21-23). To date, genetic association studies of SLE patients with and without CVD have been limited in size and have detected only a few common genetic risk loci, including IRF8, STAT4, IL19, and SRP54-AS1 (22, 24, 25). Mendelian Randomization (MR) is a causal inference method using genotypes as “treatments” when randomized controlled trials are not feasible. By measuring and correlating the effect sizes of exposure-associated genetic variants in large-scale genetic association studies on traits of interest, a causal effect of the exposure on the outcome can be estimated. Here, we report the application of multiple, complementary MR methods to identify causal paths from SLE-associated variants to CAD using summary statistics from genetic association studies. Using multiple MR algorithms, we have identified large sets of SLE causal variants that also impart genetic risk for CAD, as well as those that appear to diminish the risk of CAD. Using novel approaches to build molecular pathways from genetic risk factors (26), we have developed a map of SLE-derived biologic processes with causal implications on CAD that may account for the genetic basis of the association between these two apparently dissimilar clinical entities and may also provide novel insights into the shared mechanisms underlying each. Understanding the pathogenesis of genetic variants underlying the increased CAD risk in SLE can ultimately provide insight into the immune and inflammatory components of atherosclerosis, as well as reveal opportunities for targeted therapeutics.

### Results

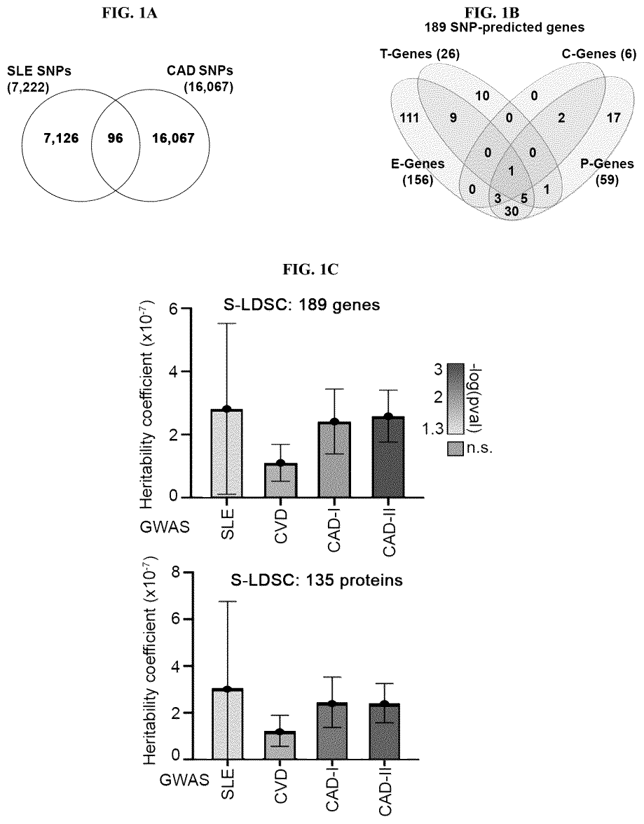

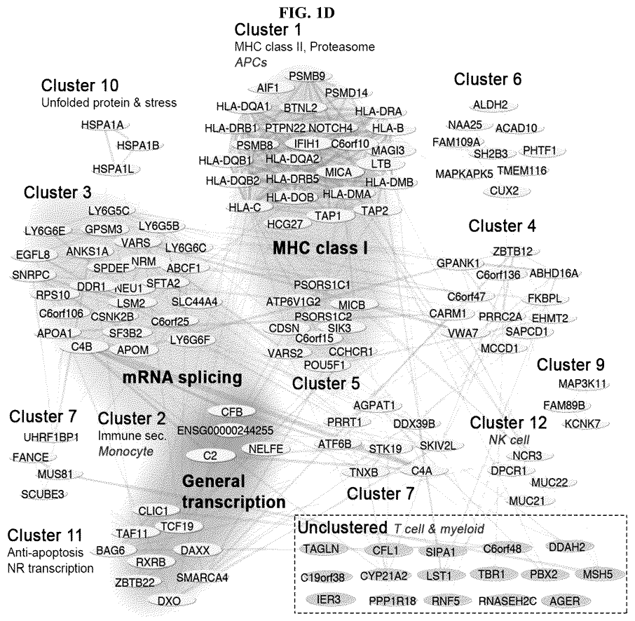

Pathway analysis reveals gene networks implicated by genetic variants associated with both SLE & CAD. To explore the shared genetic predispositions for SLE and CAD, we first identified single nucleotide polymorphisms (SNPs) associated with each trait in multi-ancestral genetic association studies(27-32). In total, 96 SNPs were associated with both conditions (FIG. 1A). To identify putative gene(s) influenced by each of the 96 SNPs associated with both SLE and CAD, we mapped causal SNPs to genes(26), identifying 189 unique genes encoding 135 proteins in STRINGdb (FIG. 1B). Stratified linkage disequilibrium score regression (S-LDSC) was then used to validate the biological relevance of SNP-predicted gene and protein sets by assessing whether they captured more disease heritability than expected by chance with respect to all genes and STRINGdb proteins, respectively(33). Application of S-LDSC using GWAS summary statistics for SLE (GCST003155(27)), CVD (GCST004280(34)) and two CAD datasets (CAD-I, GCST000998(35) and CAD-II, GCST001479(36)) determined that the 189 genes predicted by the 96 overlapping SNPs were significantly (p<0.05) enriched for genomic regions capturing the genetic heritability of CAD and CVD (FIG. 1C). Nearly identical results were also obtained using the smaller subset of 135 protein-coding genes (FIG. 1C).

To assess molecular networks encoded by this set of 135 protein encoding genes, a protein-protein interaction (PPI) network was generated and unsupervised clustering revealed 12 distinct gene clusters of variable sizes that were functionally enriched in a diverse range of immunological and cellular categories (FIG. 1D). Notably, gene clusters with relevance to SLE and CAD/CVD were also identified, including cluster I characterized by canonical pathways for Antigen presentation pathway and B cell development, along with Sudden cardiac death and cluster 3 enriched in Atherosc.Crosis signaling and Lupus erythematosus, systemic, arnongst others (Table 1). Although the molecular pathways associated with SNP-predicted genes suggested a convergence of biological processes underlying SLE and CAD, it remained unclear whether variants associated with both conditions confer similar or opposing effects on SLE and CAD. Importantly, the majority of overlapping SNPs were located in HLA region of chromosome 6.

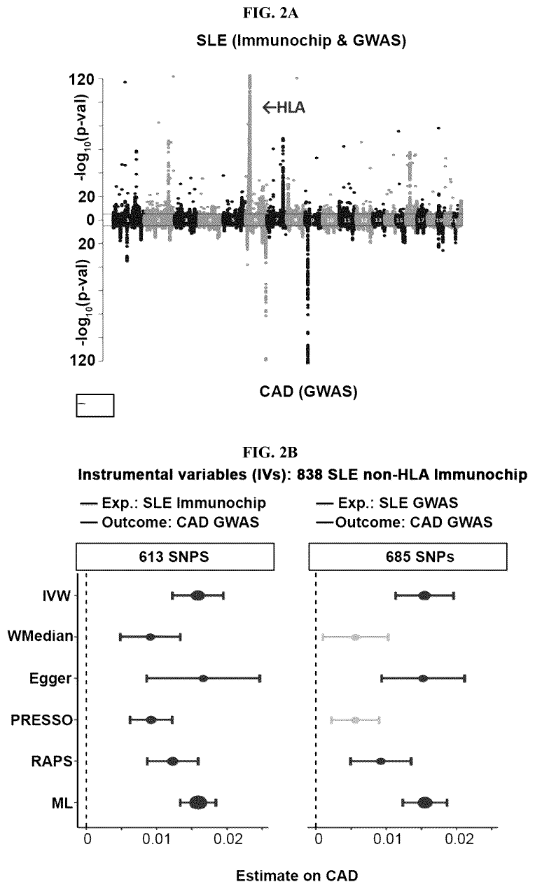

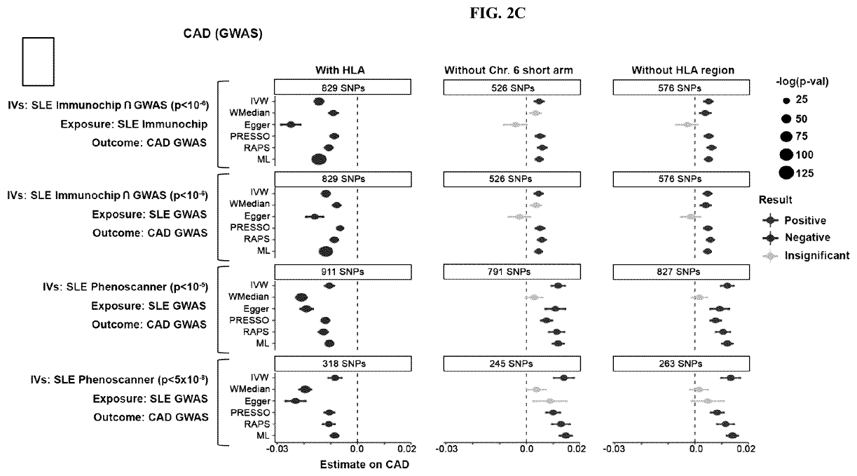

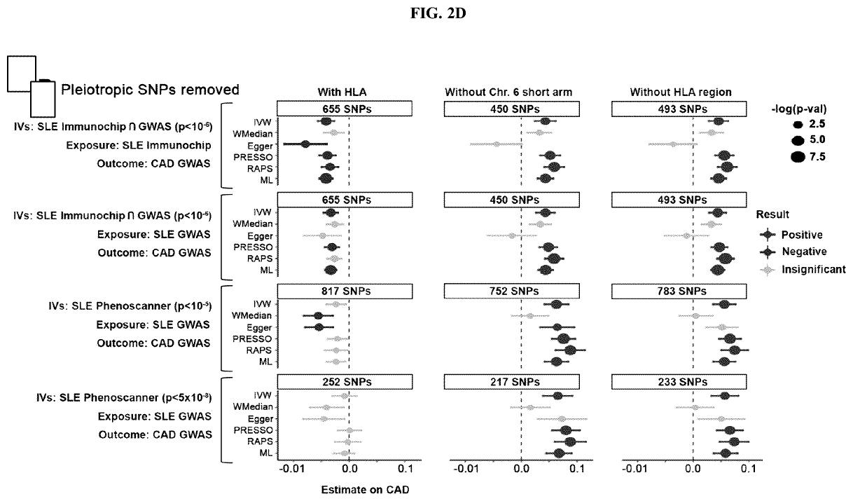

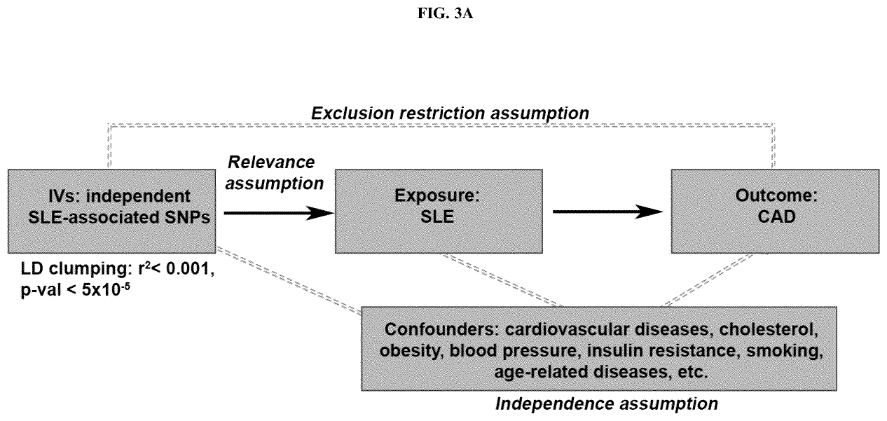

MR estimates a positive correlation between effects of SLE-associated non-HLA variants on SLE and CAD. Next, MR methods were employed to estimate the association between effect sizes of relevant variants on SLE and CAD. We first applied six MR methods using various sets of SLE-associated instrumental variables (IVs) to determine whether they tend to confer similar (positive association) or opposing (negative association) effects on SLE and CAD, noting that this initial approach did not satisfy all assumptions for IV-validity or IV-independence and therefore could only provide an estimated association. These analyses, however, suggested a net-positive correlation for non-HLA SLE-associated SNPs on CAD and a net-negative correlation between effect sizes when including the HLA region (FIG. 2A-2C). Even when SLE-associated SNPs with known pleiotropic associations to CVD or confounders such as cholesterol, obesity, blood pressure, insulin resistance and smoking were removed, the indication of a positive causal relationship between SLE and CAD remained (FIG. 2D).

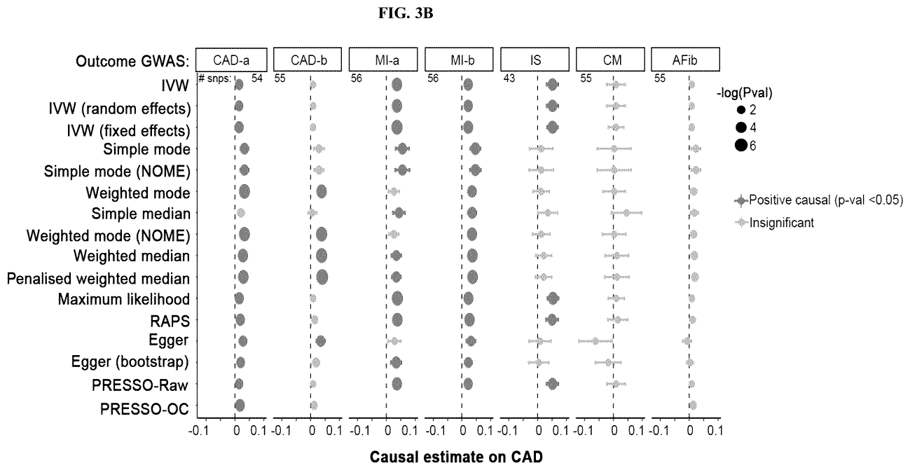

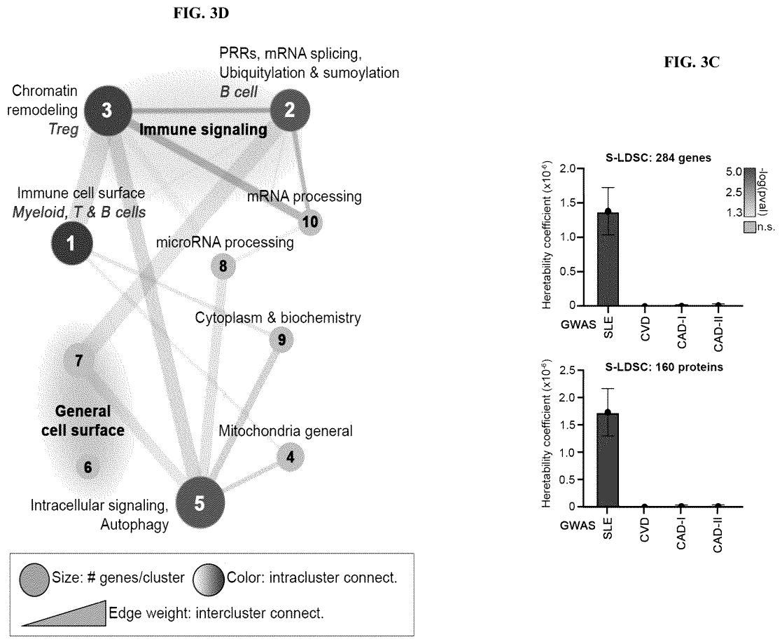

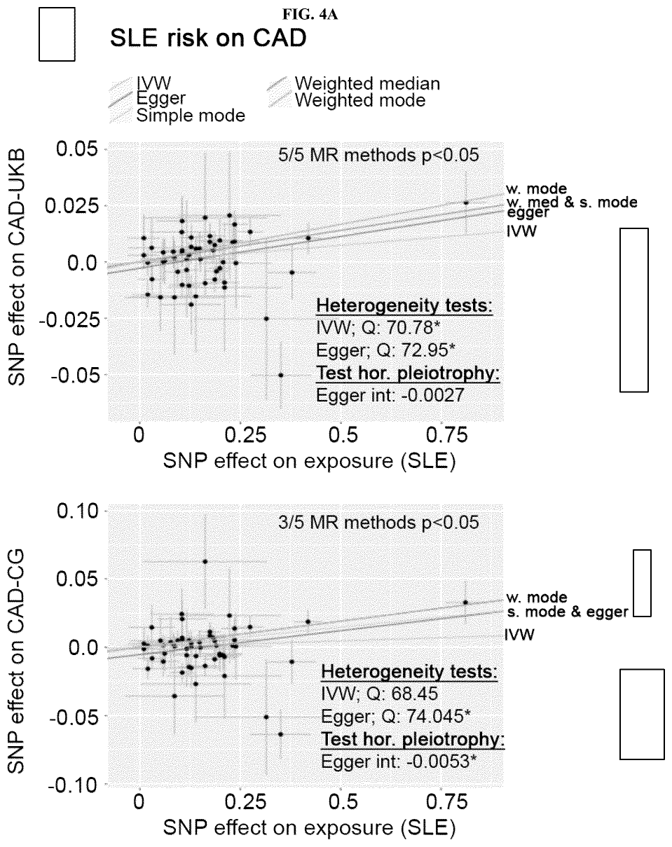

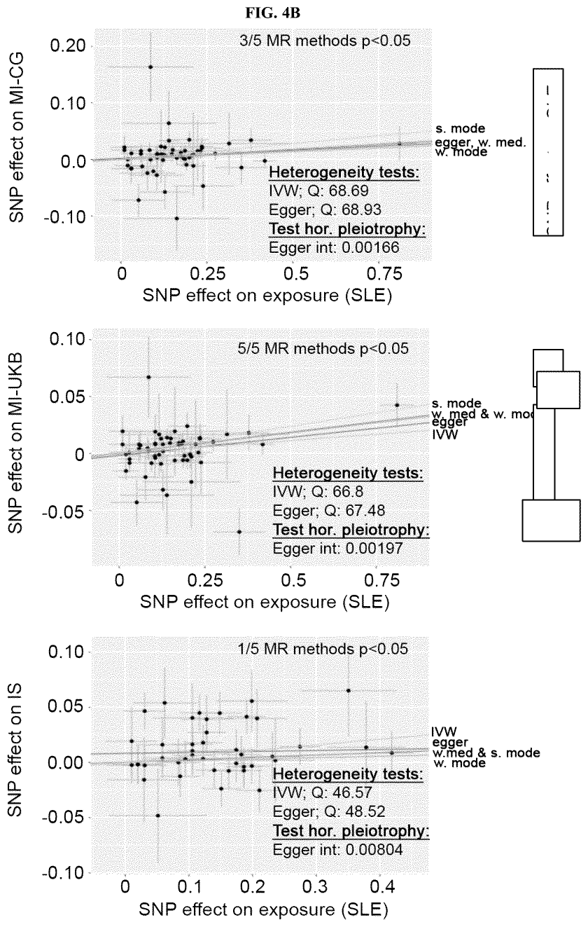

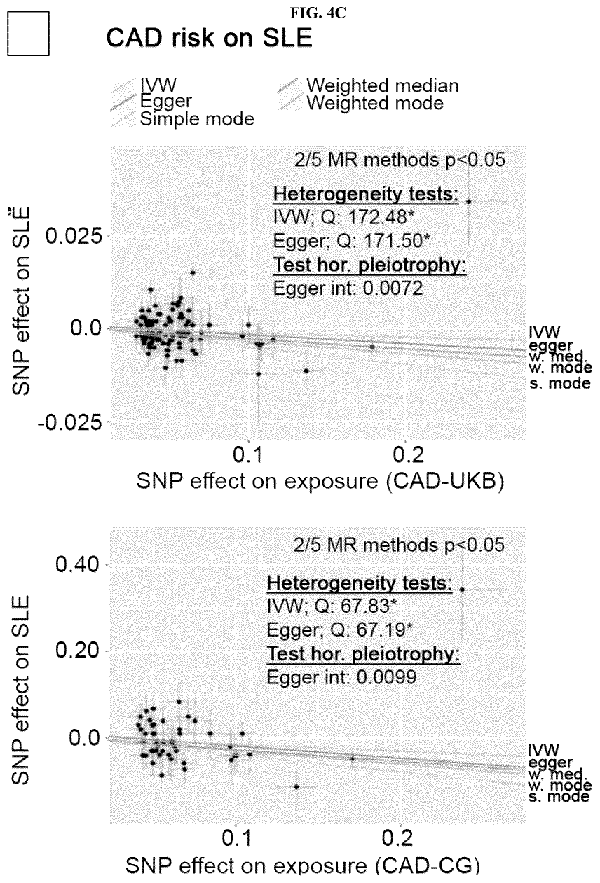

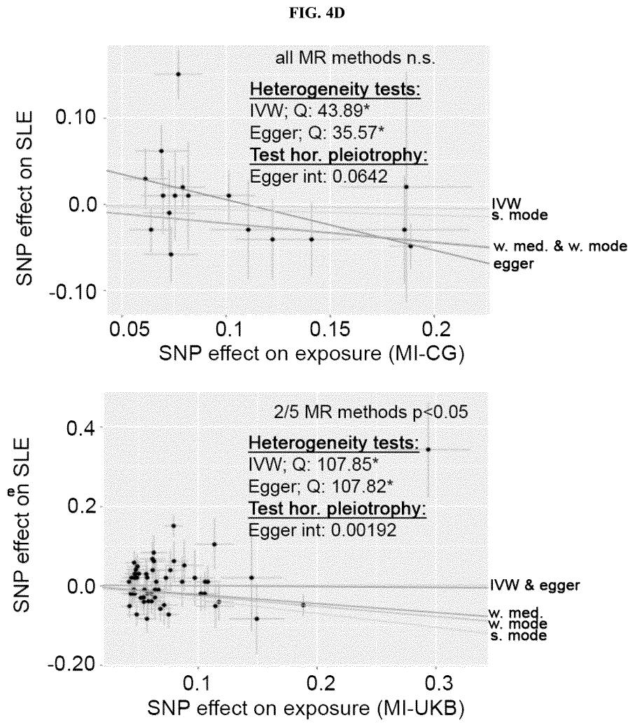

To validate the robustness of our estimated associations by satisfying the MR assumptions, we carried out two-sample MR analyses using multi-ancestral, non-HLA SNPs strongly associated (p<5×10⁻⁸) with SLE, excluding SNPs weakly associated (p<10⁻⁵) with CVD or confounders (Table 2A-2C), followed by stringent LD-clumping to ensure IV-independence (37) (R²=0.001, 100 kb window, 1000G EA reference population) (FIG. 3A). SLE GWAS summary statistics were used for exposure (27), and multiple CAD GWAS were used for outcome (GCST005194,CAD-a and GCST005195, CAD-b (32)). Since CAD is causative of myocardial infarction (MI) and atherosclerosis is common to CAD and ischemic stroke (IS), MR was carried out using summary statistics for 2 additional MI GWAS (GCST003117, MI-a (38) (GCST011365, MI-b (39)) and IS (GCST006906 (40)). Summary statistics for cardiomyopathy from the FinnGen biobank analysis (finn-b-19_CARDMYO, CM) and atrial fibrillation (GCST006414, AFib (41)), which are not associated with atherosclerosis or CAD, were also included for comparison. After LD-clumping, 60 independent SNPs were included in the SLE GWAS and then harmonized with each outcome-GWAS pair before use as IVs for SLE exposure (Table 9A-9F). Between 43-56 harmonized IVs for SLE-exposure were then tested using 16 MR methods, some of which account for additional IV-invalidity, pleiotropy, or heterogeneity, to estimate causal relationships with the various atherosclerotic and cardiac conditions. The majority of MR methods resulted in significant (p<0.05) positive causal estimates of SLE-associated variants on CAD-UKB, and both MI GWAS, but not for cardiomyopathy or Afib (FIG. 3B, FIG. 4A, Table 3A-3H). Directional pleiotropy was only detected between the SLE and CAD-CG GWAS by the MR-Egger intercept test (FIG. 4A), indicating potential bias in the causal estimates based upon effect sizes using the CAD-CG GWAS. Overall, IVW, weighted median, penalized weighted median, maximum likelihood, RAPS and PRESSO were significant in 4 out of 5 outcome GWAS (FIG. 3B). These results establish a positive causal effect of SLE on CAD and suggest that the increased CVD-risk associated with SLE is likely to involve atherosclerosis rather than other aspects of cardiac pathology.

To eliminate the possibility that the positive causal estimate of SLE on CAD is bidirectional and therefore unlikely to represent a true causal relationship, MR was also carried out in the reverse direction, with CAD or MI as exposure and SLE as the outcome. Importantly, none of the 14 methods yielded a significant positive causal estimate of CAD or MI on SLE. Of interest, however, significant (p<0.05) negative causal estimates of CAD and MI on SLE were observed for approximately half of the 14 MR methods tested (FIG. 4B, Table 3A-3H).

To understand the pathways underlying the positive causal estimates of SLE on CAD in greater detail, all SLE-associated SNPs included as putative IVs before harmonization with each GWAS were mapped to genes. Consistent with satisfying the exclusion restriction criteria and independence assumption with respect to traits imposing significant CVD-risk, S-LDSC results demonstrated that the 284 genes and 160 predicted proteins captured a significant portion of SLE heritability (p-values=3.46×10⁻⁵ and 3×10⁻⁵, respectively), but not that of CVD or CAD (FIG. 3C).

Proteins predicted from the SLE IVs were then integrated into connectivity networks in STRINGdb (FIG. 3D). Cluster annotations were dominated by processes commonly dysregulated in SLE as expected, including canonical pathways for Systemic lupus erythematosus in B cell signaling, Th1 pathway and Th2 pathway, as well as GO terms for Regulation of immune response (GO:0050776) and Negative regulation of B cell activation (GO:0050869) (Table 4). Interestingly, disease associations were enriched in various autoimmune diseases (Lupus erythematosus, systemic, Aicardi goutiere's syndrome and Hashimoto's disease), along with cardiovascular dysfunction, such as Arterial embolism and thrombosis, Hypertension, Plaque, atherosclerotic and Ischemic heart disease (Table 4).

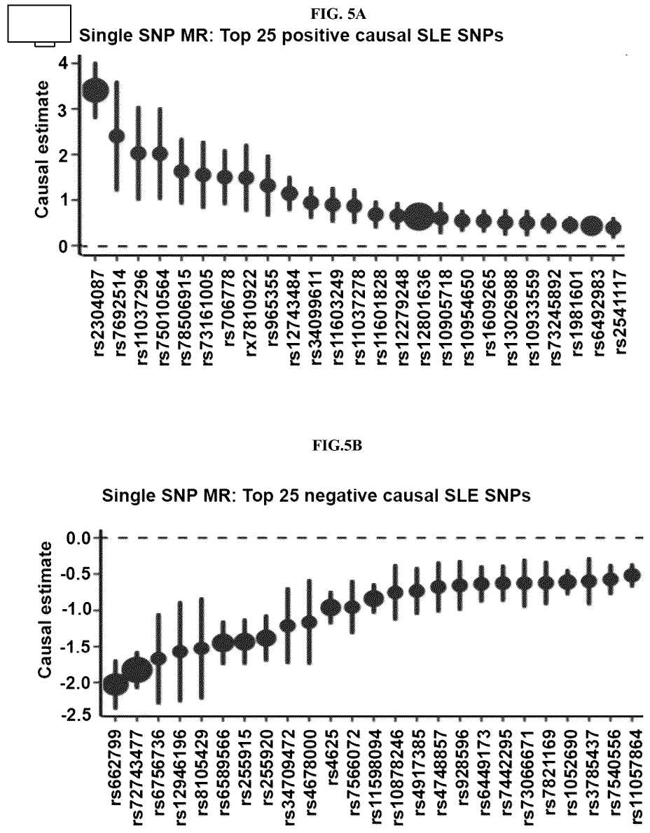

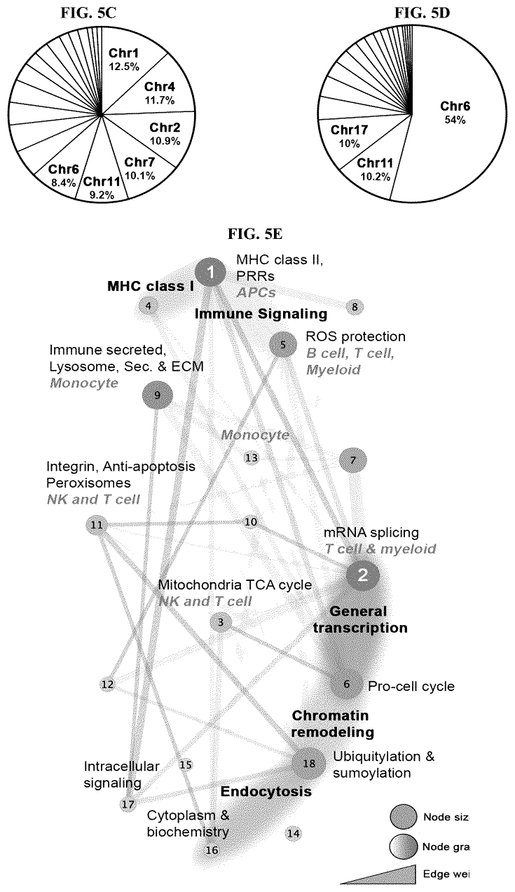

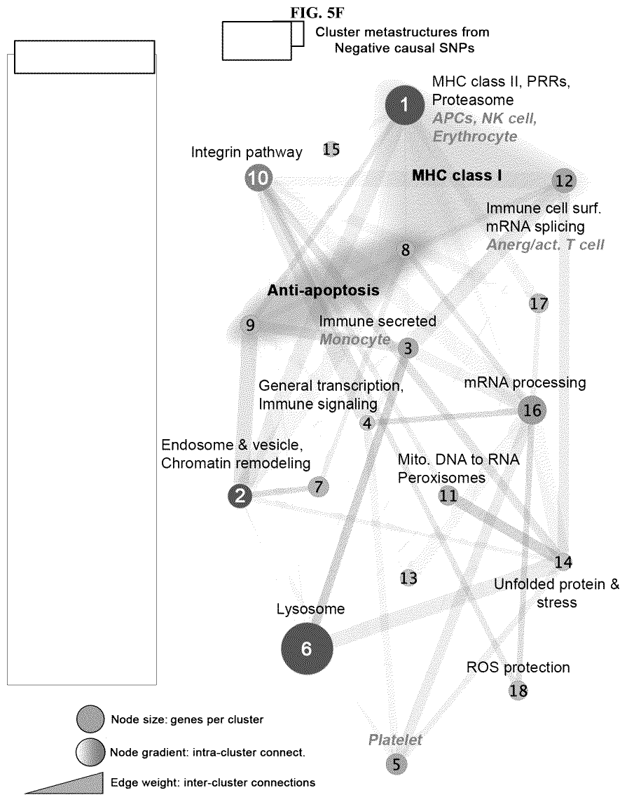

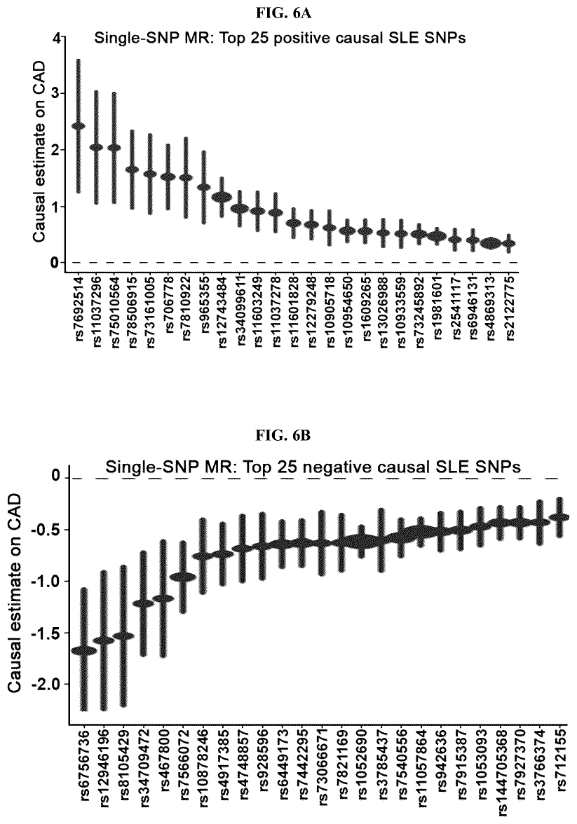

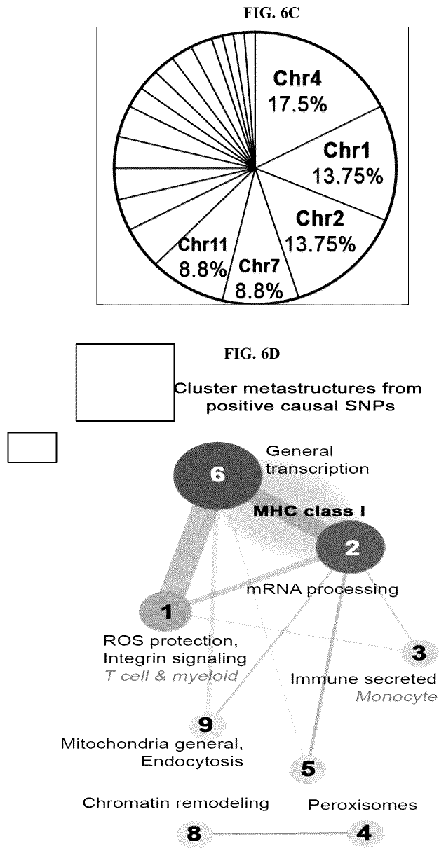

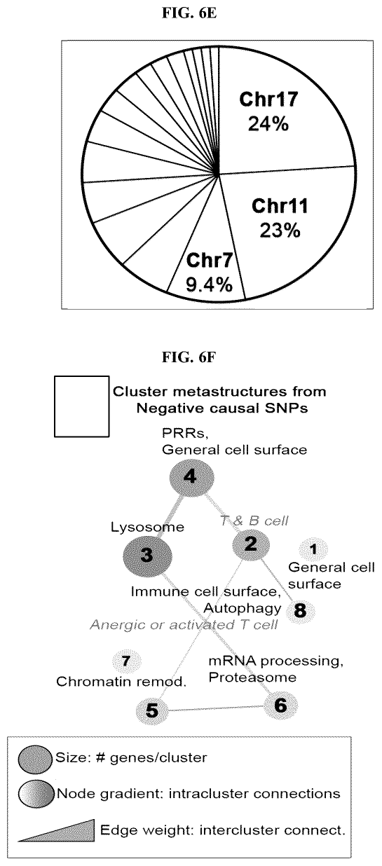

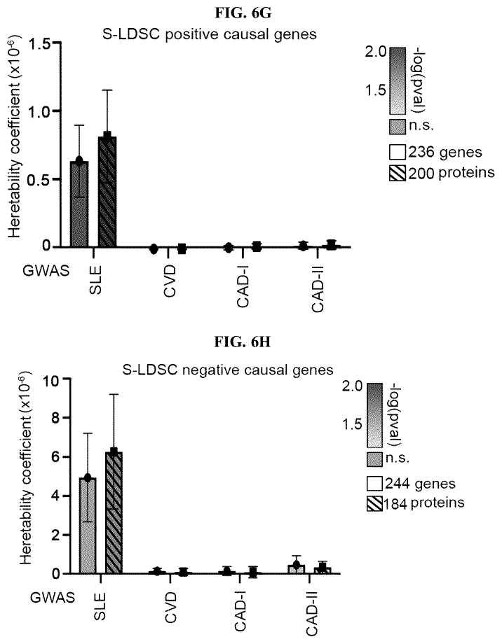

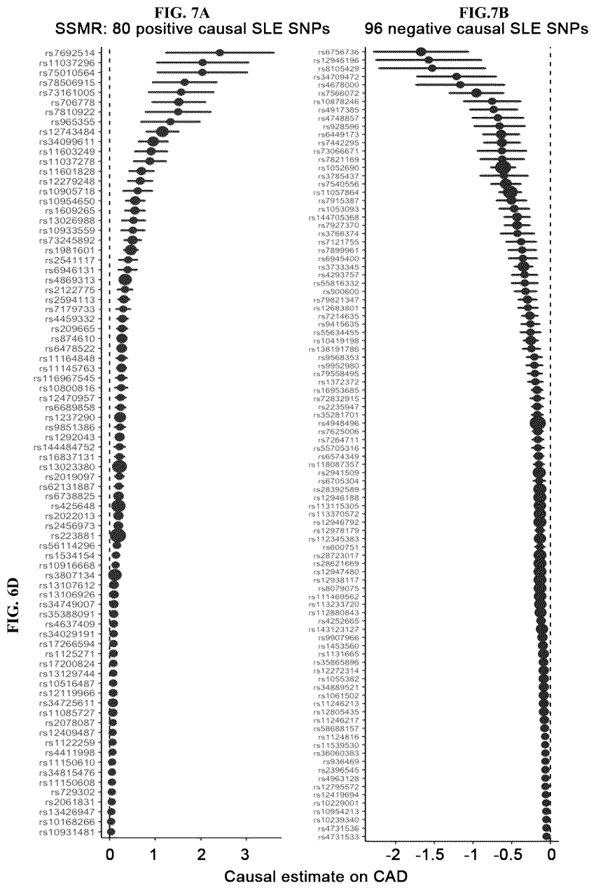

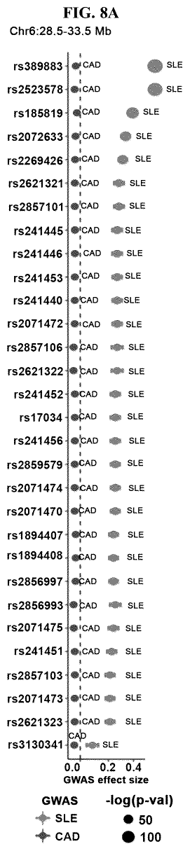

Single-SNP MR identifies gene networks implicated by SLE-associated variants with positive and negative causal estimates on CAD. We next employed single-SNP MR (SSMR) to identify specific SLE-associated variants with positive or negative estimates on CAD. SSMR applied to SLE-associated SNPs, including those in the HLA region, reveal that the majority of negative causal SNPs are located on the short arm of chromosome 6; all but one were tightly packed around the HLA region, spanning chr6:28014374-33683352 (FIG. 5A-5F). When excluding the short-arm of chromosome 6 and SNPs associated with CVD or confounders, SSMR identified 80 and 96 SLE-associated variants with significant (p<0.05) positive (FIG. 6A, top 25) and negative (FIG. 6B, top 25) causal estimates on CAD, respectively (FIG. 7A-7B, Table 3A-3H). The majority of positive-causal SNPs were distributed on chromosomes 1, 2 and 4, whereas over 50% of negative causal SNPs were on chromosomes 7, 11 and 17 (FIG. 6C, 6E).

Non-HLA SLE variants with either significant positive or negative causal estimates on CAD were separately mapped to 236 (FIG. 6D) and 244 predicted genes (FIG. 6F), respectively, for clustering and pathway analysis. Positive SNP-predicted gene clusters were enriched in the canonical pathway for Antigen presentation and functional categories for MHC class I, as well as epigenetic processes, transcription, and endocytosis (FIG. 6E, Table 5A-5B), whereas negative SNP-predicted gene clusters were dominated by processes related to cell death (Pyroptosis (GO:0070269), cluster 2; Regulation of oxidative stress-induced cell death (GO:1903201), cluster 6) and protein degradation (Proteasome, cluster 6 and Autophagy, cluster 8; FIG. 6F, Table 5A-5B). Finally, gene sets predicted by both positive and negative causal SNPs captured a significant portion of SLE heritability, but not that of CAD or CVD, consistent with their selection as IVs for SLE (FIG. 6G-6H).

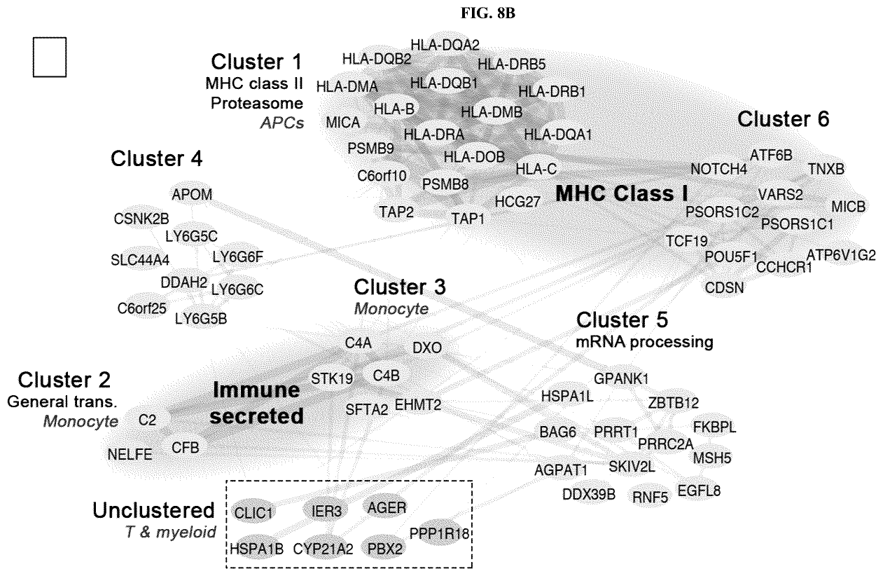

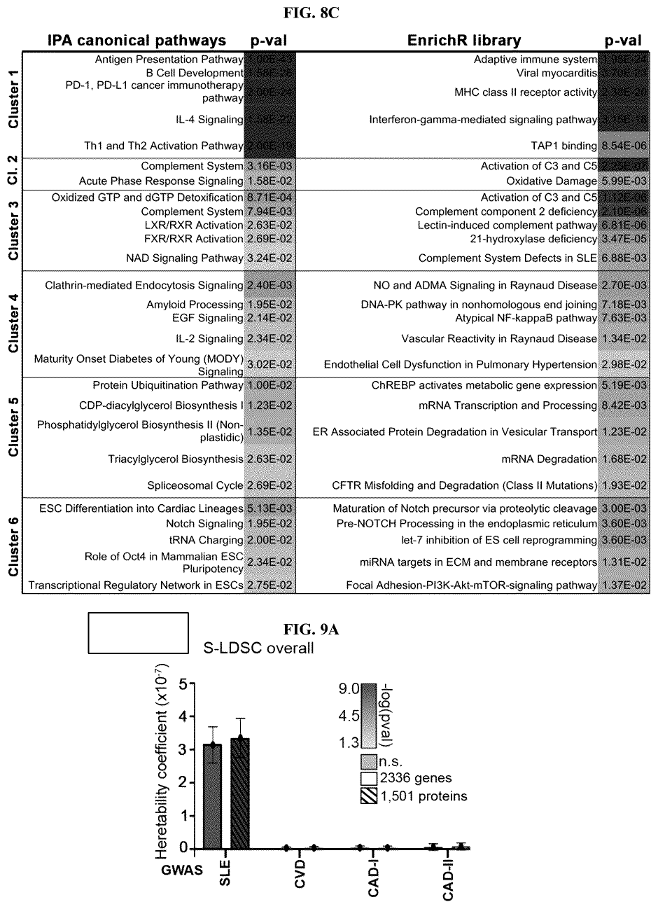

Pathway analysis of HLA region variants associated with SLE-risk and protective of CAD. Risk haplotypes in the HLA region heavily contribute to susceptibility for SLE (42) and CAD (43). However, accurate genotyping of HLA alleles and corresponding GWAS effect size estimates are notoriously unreliable (44). Additionally, the complex genetic architecture of this region makes mapping HLA variants to genes especially challenging given the extensive LD and high density of genes in this region. Nonetheless, an examination of the HLA area (chr6:28.5-33.5 Mb) revealed 30 SNPs significantly (p<10⁻⁶) associated with both SLE and CAD in their respective GWAS. While these SNPs are not independently associated variants, all 30 SNPs had positive effect sizes for SLE but were negative for CAD (FIG. 8A), possibly reflecting the extensive LD in this region. Connectivity mapping and clustering of the 69 protein-encoding genes predicted from these 30 SNPs revealed 6 distinct clusters dominated by processes dysregulated in SLE, including the functional categories for MHC class I and MHC class II in clusters 1 and 6, along with canonical pathways for TH1 and TH2 activation, B cell development, Notch signaling as well as gene ontogeny (GO) terms for Interferon-gamma mediated signaling pathway (GO:0060334) (FIG. 8B-8C). Other pathways of interest involving Complement system abnormalities, LXR RXR activation, and 21-hydroxylase deficiency were predicted by cluster 3 (FIG. 8C).

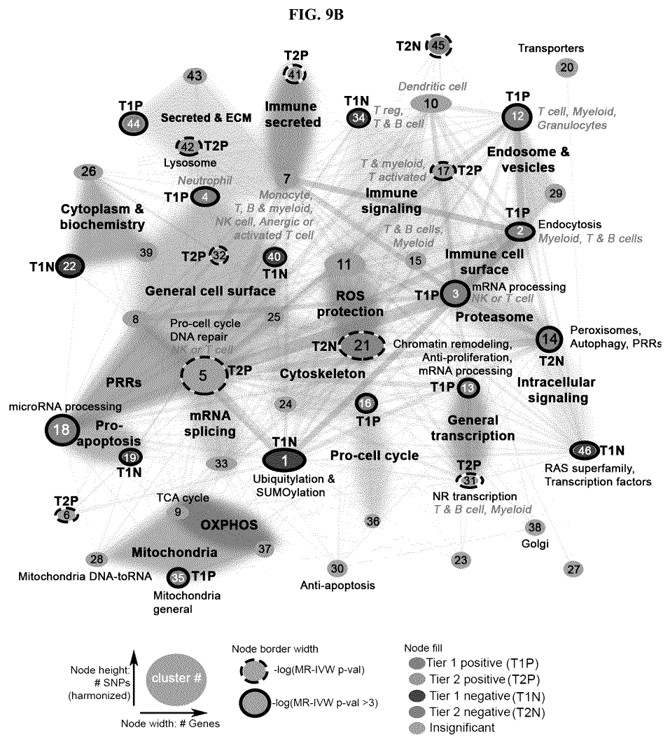

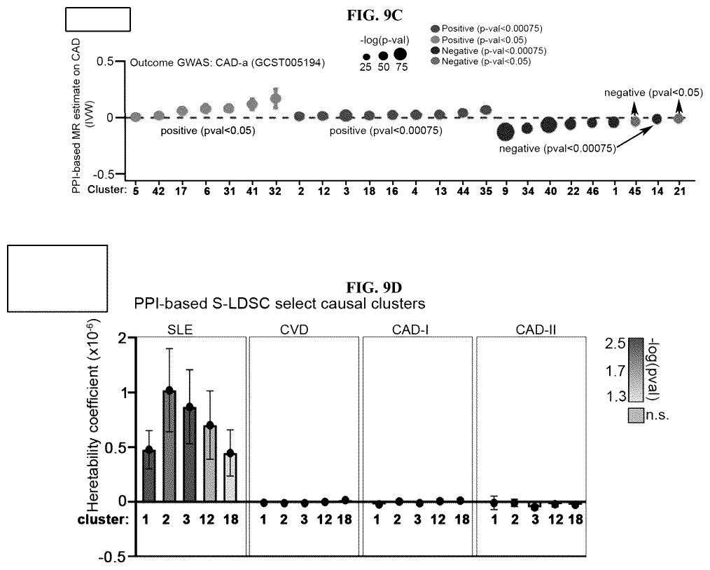

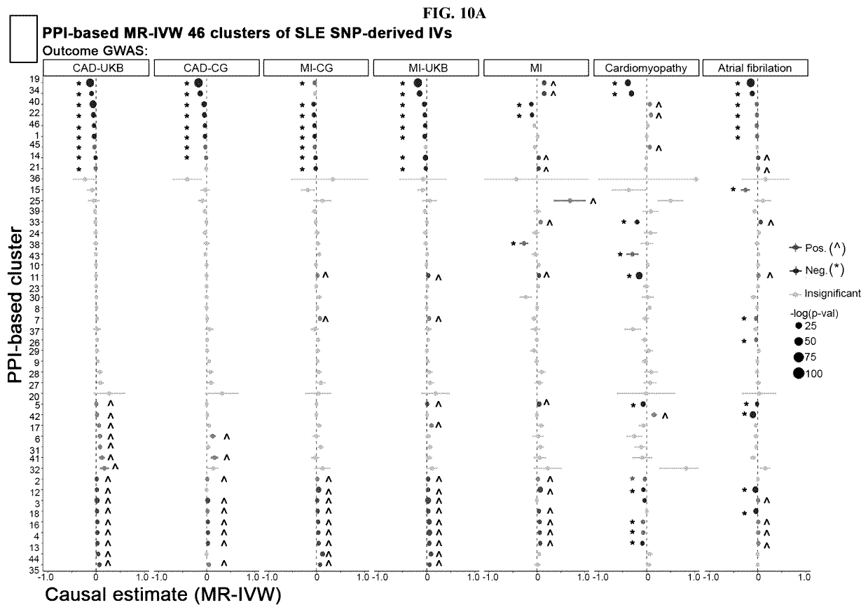

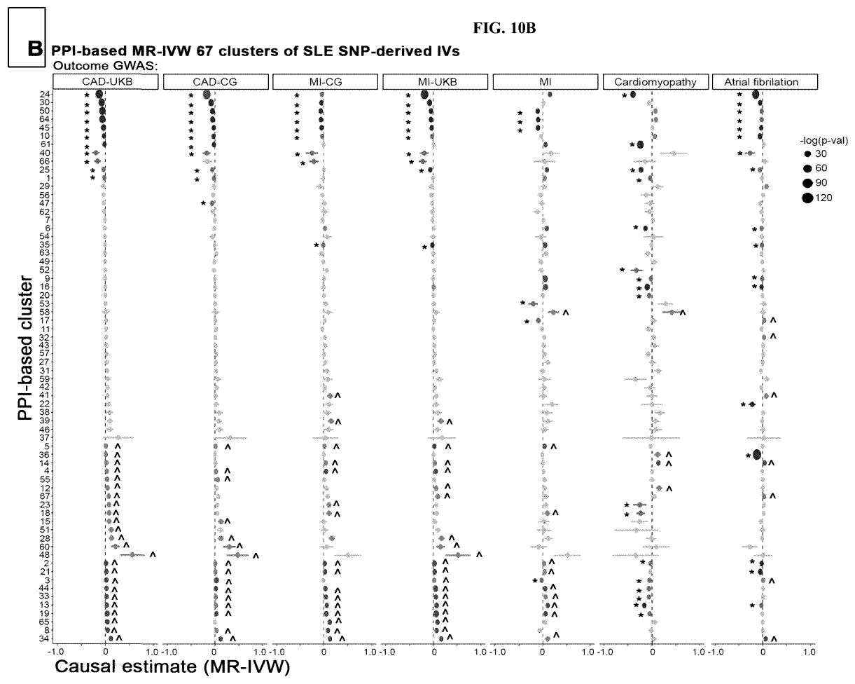

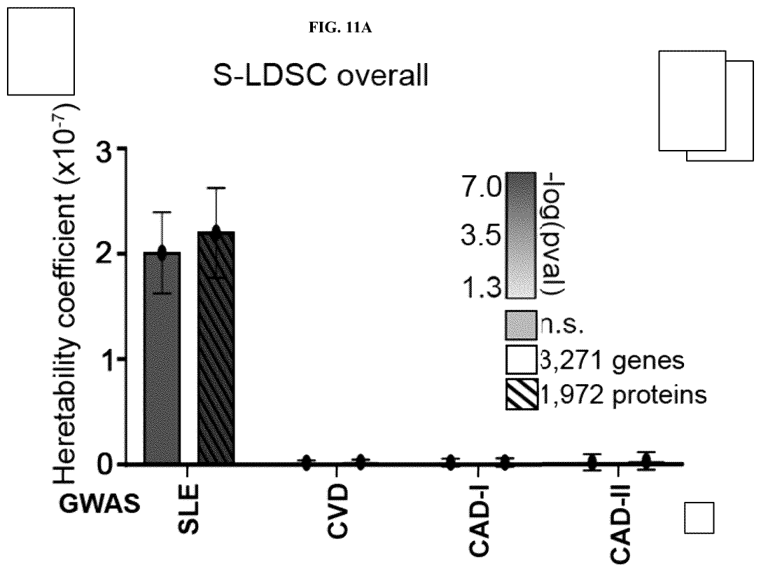

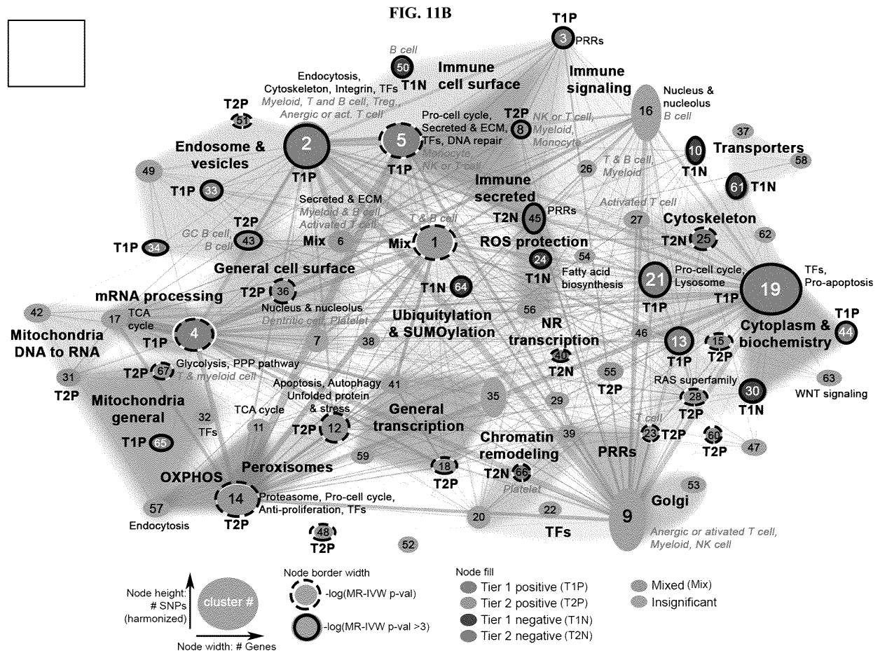

PPI-based MR predicts specific sets of SLE-associated variants and gene pathways causal of CAD. To obtain a more comprehensive view of the possible impact of SLE-derived molecular pathways on atherosclerosis, we mapped SLE-associated, non-HLA Immunochip SNPs with net positive causal estimates on CAD by MR to genes and pathways regardless of their associations with CVD-related traits. In total, 838 SNPs predicted 2,336 putative genes and 1,501 proteins that collectively captured a significant amount of SLE, but not CAD or CVD, heritability (FIG. 9A); these 1,501 proteins clustered into 46 distinct clusters based on PPI connectivity (FIG. 9B). We then grouped SLE-associated SNPs mapping to genes in each of the 46 PPI-based clusters for use as SLE-IVs to estimate cluster-specific associations with atherosclerotic traits. Initial application of MR-IVW to these 46 subsets of SLE SNP-derived IVs yielded 16 and 9 significant (p<0.05) positive and negative causal estimates, respectively (FIG. 9B-9C, FIG. 10A) for CAD. Additional MR methods, including mode and median based methods, MR-Egger, MR-RAPS, MR-PRESSO, and maximum likelihood, were carried out for further validation of the PPI-based MR-IVW causal estimates (Table 3A-3H). Clusters were grouped into tiers with respect to consistency across the various MR methods, with tier 1 clusters yielding significant positive or negative causal estimates by almost all (at least 14/16) MR-methods and tier 2 clusters yielding significant positive or negative causal estimates by MR-IVW or at least 7 MR-methods (FIG. 9B-9C). Finally, when examined individually, 22 of the 46 clusters specifically captured SLE heritability by PPI-based S-LDSC, many with significant causal estimates on CAD by PPI-based MR (FIG. 9D and Table 6A-6H).

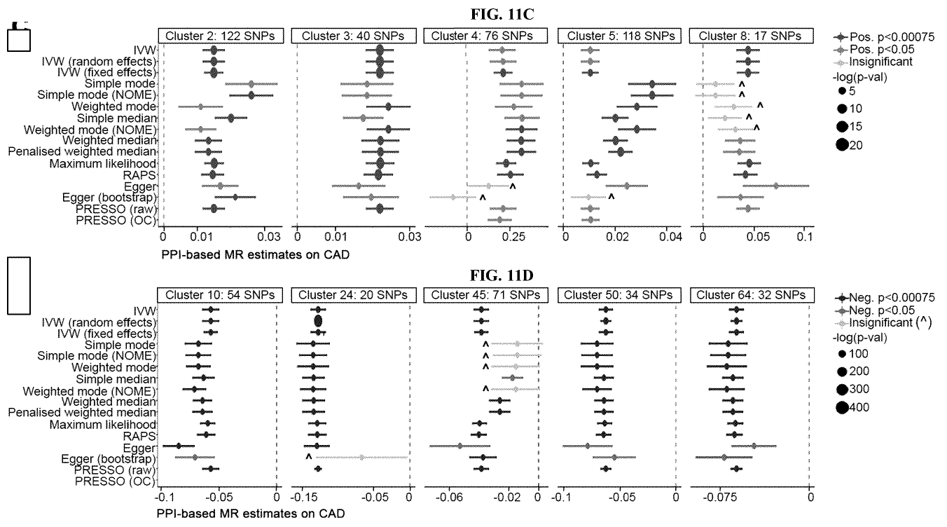

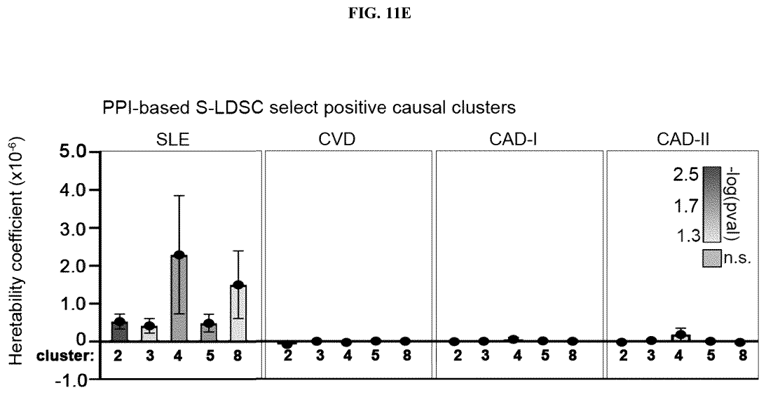

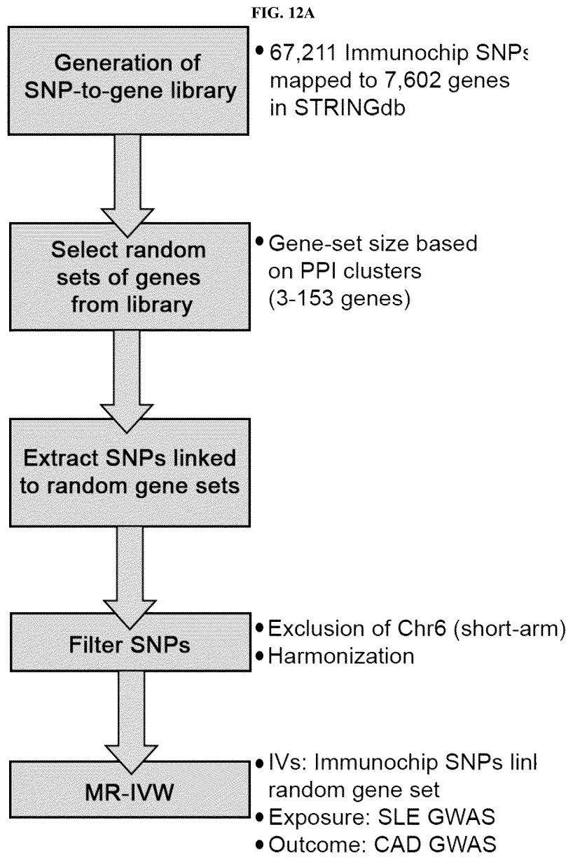

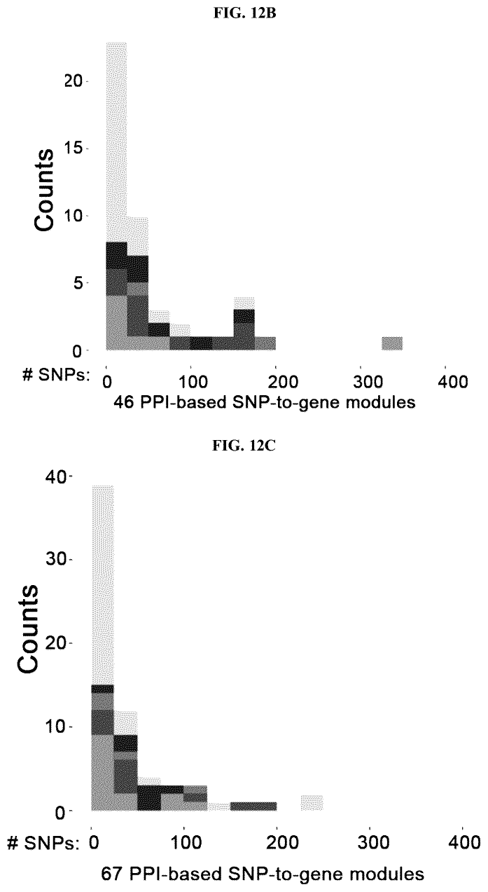

In an effort to support these results by expanding the size of the network, we added 914 multi-ancestral, non-HLA SNPs associated with SLE on the Phenoscanner database to the analysis. Overall, 1,708 unique SNPs predicted 3,272 putative genes and 1,972 proteins that collectively captured a significant amount of SLE heritability, but not that of CAD or CVD (FIG. 11A) and clustered into 67 distinct sets of protein-coding genes (FIG. 11B). PPI-based MR-IVW using these 67 clusters of SLE SNPs as IVs yielded 24 and 11 significant (p<0.05) positive and negative causal estimates on CAD, respectively (FIG. 11C-11D, FIG. 10B), many of which captured SLE heritability, but not that of CAD or CVD by PPI-based S-LDSC (FIG. 11E and Table 6A-6H).

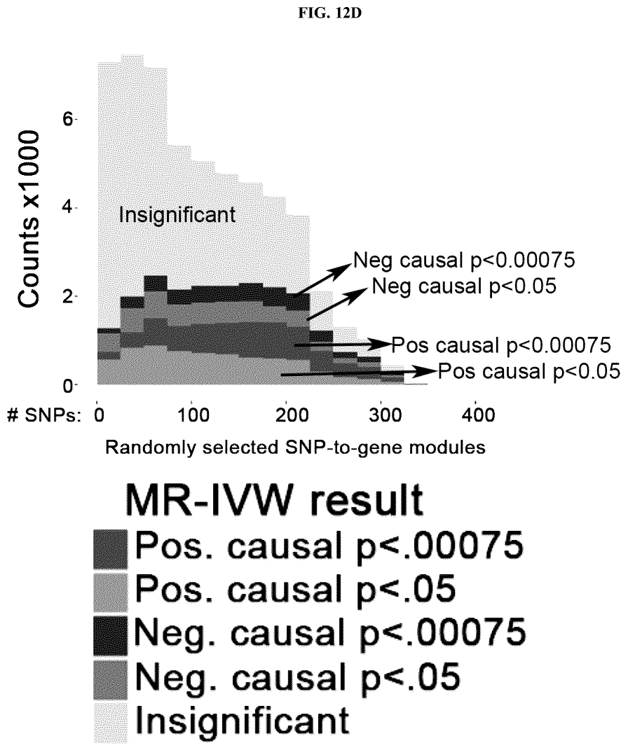

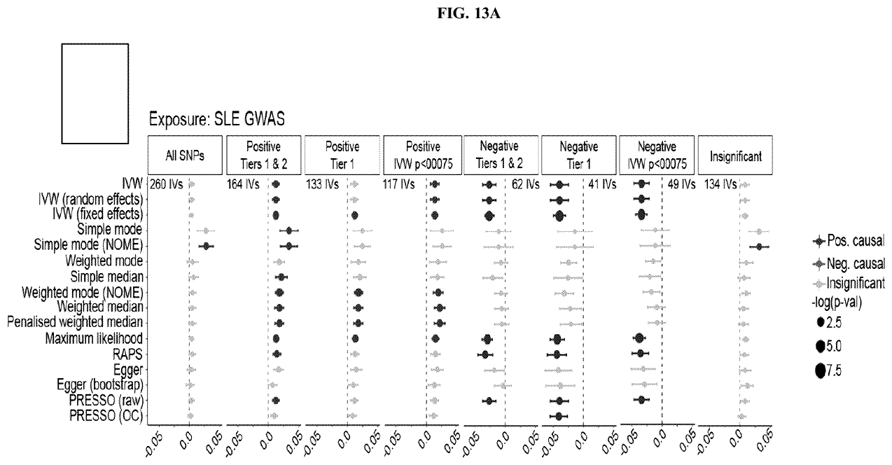

To ensure that the majority of predicted causal clusters are not a result of random chance or multiple-hypothesis testing, we carried out simulations to estimate the false discovery rate with respect to our PPI-based MR approach. A total of 67,211 unique Immunochip SNPs mapping to 7,602 STRINGdb genes were used to generate a SNP-to-gene library. In each simulation, groups of 3 to 152 SNP-predicted genes were randomly selected from the library; up to 400 SNPs mapping to each random gene set were then extracted to generate subsets of SNPs for use as IVs. MR-IVW was carried out for SLE on CAD using these randomly generated SNP-to-gene modules after harmonization and exclusion of HLA region variants (FIG. 12A-12D). Using a Bonferroni-corrected p-value of <0.00075 (0.05/67 clusters), a substantially lower false discovery rate was noted. Importantly, these simulations demonstrate an increase in the false discovery rate of PPI-based MR-IVW as the number of SNPs used as IVs increase, which is likely due to LD. By using our Immunochip derived SNP-to-gene dictionary for random selection of protein clusters and associated SNPs as IVs, our simulations account for both a high degree of LD and pleiotropy and importantly demonstrate a higher rate of significant causal estimates using SLE-derived, as opposed to randomly generated SNP-to-gene modules on CAD.

To assess the reproducibility of the cluster-specific causal estimates, PPI-based MR was repeated using CVD-related GWAS datasets on the MR-base platform (45). The PPI-based MR-IVW causal estimates were highly consistent using summary statistics from 2 CAD and 2 MI GWAS on MR-base, but not cardiomyopathy or AFib (Table 7A-7B), suggesting that the stratified causal estimates on CAD are associated with the atherosclerotic component of CVD. Together, these results support the conclusion that the PPI-based MR results are atherosclerosis-specific and unlikely trivial results of random chance or multiple hypothesis testing.

SLE-derived clusters in all positive and negative causal tiers were annotated using multiple functional and cellular composition tools (FIG. 11B, Table 8A-8E). These results show that a wide range of SNP-predicted biological functions known to be involved in SLE pathogenesis have causal implications on CAD by MR, such as Neutrophil degranulation (clusters 2 and 43), Th1 and Th2 activation, and/or Th17 activation (clusters 3, 5, 8, and 9), Interferon signaling (cluster 8), Leukocyte extravasation signaling (cluster 12), Leukocyte trans-endothelial migration (cluster 28) and Leukocyte adhesion to endothelial cells (cluster 2). In addition to immune-related pathways, many of these positive causal clusters were enriched in disease phenotypes associated with cardiovascular disease, including Th1 cell activation and proliferation in atherosclerosis (cluster 9) and Lipid and atherosclerosis (cluster 12). Interestingly, several clusters were enriched in autonomic nervous system control related to cardiac function (Cardiac muscle contraction, clusters 13 and 33) or Neuroinflammation (cluster 60) (FIG. 11B, Table 8A-8E).

In contrast, SLE-derived clusters with negative causal estimates on CAD were enriched for oxidative stress (cluster 10), nitric oxide (clusters 24, 40, and 64), and HDL cholesterol (clusters 24 and 50) (FIG. 11B, Table 8A-8E). Pathway enrichment was further reflected in assigned functional categories, with ROS protection (clusters 24 and 45), NR transcription (cluster 40) and ubiquitylation and SUMOylation (cluster 64) dominating clusters with protective estimates on CAD (FIG. 11B). These results are highly consistent with the enrichments associated with negative causal variants in our single-SNP MR and HLA-specific pathway analyses.

PPI-based MR stratifies SNPs, genes, and networks underlying the positive and negative causal effects of SLE on CAD. To further validate the causal effects of the 67 SNP-to-gene modules identified by PPI-based MR, we carried out additional MR analyses with respect to PPI-based MR cluster-groupings after accounting for pleiotropy and LD.

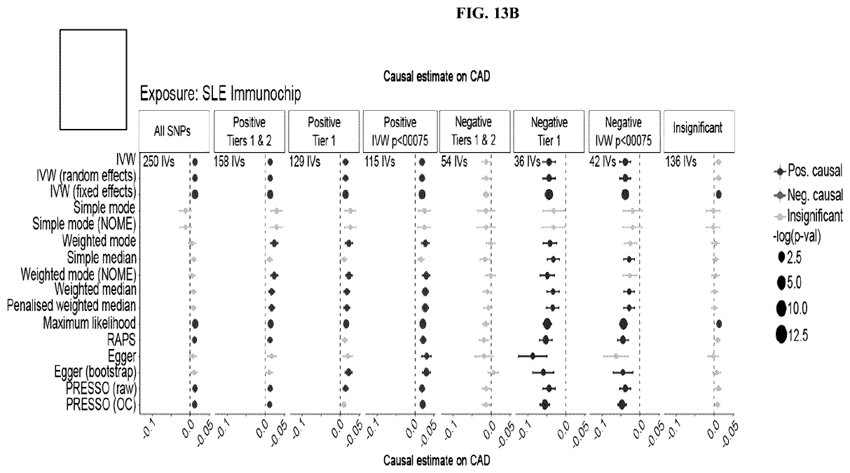

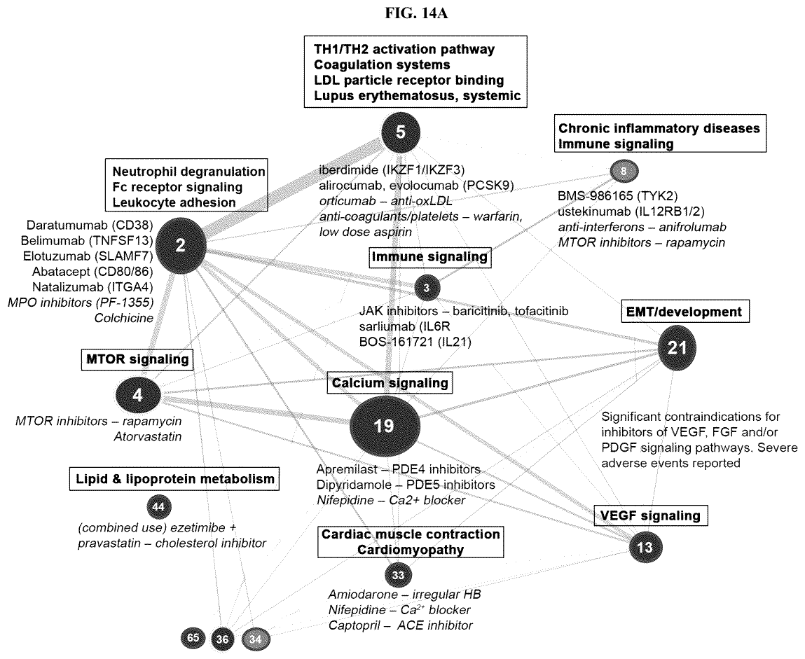

Causal estimates of SLE on CAD with IVs derived from clusters meeting the tier 1 or the tier 1 and 2 criteria, as well as those that surpassed the MR-IVW p-value <0.00075 threshold were universally more positive, significant, and consistent than those based upon all SNPs (FIG. 13A-13B, Table 3A-3H). Similarly, negative causal estimates for SLE on CAD were obtained using IVs meeting the negative tier 1 and MR-1VW p-value <0.00075 thresholds from the 67-cluster network. In contrast, IVs derived from clusters with insignificant causal estimates generally failed to reach significance in either direction. While these trends were observed using summary statistics from both the SLE GWAS and SLE Immunochip, causal estimates were more significant using the SLE Immunochip, consistent with its larger sample size. Importantly, these results demonstrate that PPI-based MR can be used to identify independent IVs satisfying MR assumptions that underly both positive and negative causal effects of SLE on CAD.

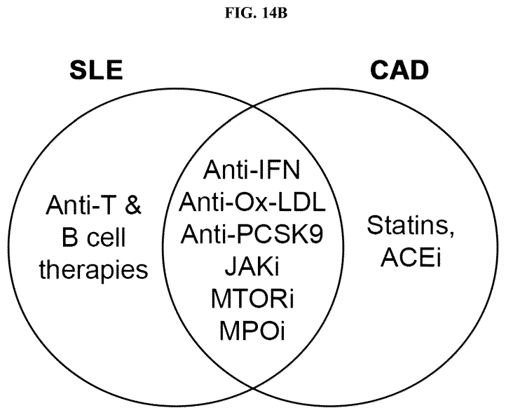

Pathway analysis facilitates drug prediction. Pathways associated with positive causal clusters were used to facilitate identification of new therapeutic interventions for managing the unique inflammatory environment contributing to CAD in SLE (FIG. 14A). Canonical pathways related to immune function in clusters 2, 3, 5 and 8 predicted drugs targeting T and B cells and inflammatory cytokines, including daratumumab (CD38), belimumab (TNFSF13), elotuzumab (SLAMF7), abatacept (CD80/86), iberdimide (IKZF1/IKZF3) and sarliumab (IL6R). Broader analysis of pathway categories also suggested the utility of targeting interferon signaling with anifrolumab (cluster 8), as well as anti-platelet/coagulant therapy to combat dyslipidemia (cluster 5) (46). Additional noteworthy targets include PCSK9 (cluster 5), a protease involved in the degradation and recycling of the LDL receptor targeted by alirocumab and evolocumab, and oxidated LDL molecules (cluster 5) targeted by orticumab (FIG. 14B). Table 13 lists the SNPs within, the functional annotation of, and mapped genes of the 67 SNP clusters. SNPs within, the functional annotations of, and mapped genes of the individaul clusters are listed in Table 13-1 to 13-67, where Table 13-X (X: 1-67) lists the SNPs within, functional annotations of, and mapped genes of cluster X of the 67 clusters, e.g., Table 13-1 lists SNPs within functional annotations of, and mapped genes of cluster 1 of the 67 clusters. For any cluster that had insignificant causal estimate, no annotation (N/A, non-applicable) is listed.

### Discussion

Although genetic association studies have been successful in mapping disease loci in both immune and cardiovascular diseases, the genetic and molecular basis for the increased CAD predisposition in SLE patients has remained largely unexplained. Considering the limited data on CAD in SLE, we developed a novel approach that utilized GWAS summary statistics for both diseases to identify and interpret various sets of SLE-associated variants with causal implications on CAD. The new findings include genetic variants associated with both traits, HLA variants with opposing effects on SLE and CAD, SLE-associated variants with positive and negative causal estimates on CAD by single-SNP MR and large sets of SLE-associated SNPs with causal implications on CAD. Moreover, the causal relationship with SLE appears to be focused on the atherosclerotic process, evidenced by positive estimates with CAD, MI and ischemic stroke, but not other cardiac conditions, such as cardiomyopathy or AFib. Furthermore, we developed and carried out a novel PPI-based MR approach to identify specific sets of SLE variants mapping to biologically relevant gene sets with causal implications on CAD. By coupling various MR methods with network modeling and variant interpretation, we not only provided substantial evidence of shared genetic risk but also identified the putative molecular pathways involved in the development of CAD in SLE. Moreover, a number of the immune and inflammatory pathways identified in these analyses could well contribute to the pathogenesis of CAD even in the absence of SLE or other recognized autoimmune conditions. This points to the larger implication that CAD itself is a heterogeneous condition and subpopulations, such as those driven by SLE-associated processes, might require potentially distinct treatment strategies, at least partially motivated by unique genetic predispositions.

Causal inference using traditional MR methods rely on strict assumptions for independent IVs, including that they are associated with the exposure, but not with the outcome or any potential confounders. Given the extensive pleiotropy underlying complex traits such as SLE and CVD, efforts to satisfy these assumptions can result in biasing the analyses by excluding previously established associations. Furthermore, the exclusion of SNPs associated with CVD-related traits results in the loss of relevant molecular information. While the use of SLE IVs that are also associated with CVD or confounders in traditional MR disqualifies the causal estimates from representing an effect on CAD directly through SLE, these SNPs can be just as important with respect to understanding the relevant biological pathways underlying CAD in SLE. Similarly, stringent LD-clumping to obtain an independent set of IVs not only reduces the statistical power of MR (47), but also can omit additional SNPs, genes, and pathways underlying CAD in SLE. Due to our rigorous efforts to satisfy the assumptions and account for LD in the traditional MR analyses, while also employing numerous MR methods that account for IV-invalidity, pleiotropy, or heterogeneity, these results may give overly-conservative estimates of the causal effects and underlying mechanisms as a result of over-pruning.

To overcome these limitations of traditional MR, we developed and employed a novel PPI-based MR approach using networks comprehensively derived from large sets of SLE-associated SNPs, regardless of their associations with CVD-related traits. By generating cluster-specific associations between effect sizes on SLE and CAD, biologically relevant SNP-to-gene modules can be categorized as having similar (positive estimates) or opposing (negative estimates) effects on SLE and CAD. Traditional MR using independent, SLE-specific IVs mapping to positive and negative clusters, separately, confirmed that the groups of causal clusters are representative of positive and negative causal effects on CAD through SLE, respectively. We believe that our PPI-based MR approach is particularly beneficial in cases when the exposure is complex and heterogeneous, such as SLE which embodies a diverse range of molecular and pathophysiological mechanisms that we expect to impose unique casual effects on CAD.

An essential component of this network modeling and variant interpretation approach is our comprehensive mapping of genetic variants to genes. Genetic variants are typically mapped to genes with respect to genomic location, identifying genes containing and/or nearby the SNPs of interest. Additionally, more recent advances have given rise to identification of trans-acting genomic regions that can epigenetically and/or transcriptionally influence genes at distant locations. This is especially important for complex, polygenic traits, such as SLE and CAD, of which most associated variants are non-coding. Here, we link SNPs to genes via amino acid changes in encoded proteins, proximity, expression quantitative trait loci (eQTL) predictions, and regulatory elements in an effort to be as comprehensive as possible. Our subsequent PPI-based clustering elucidated a broad range of biologically relevant molecular networks within the diverse set of implicated genes and importantly served to filter out noise. Furthermore, our PPI-based MR approach served to highlight SNP-to-gene modules contributing most to the causal effects of SLE on CAD. Together, these results demonstrate how SLE genetics can be used to identify both known and novel loci and pathways with causal implications on CAD.

Numerous biologically relevant SNP-to-gene sets were determined to have positive causal effects on CAD through SLE by MR, spanning inflammatory factors, adaptive and innate immunity, intracellular signaling, cell differentiation, microRNA and mRNA processing, mitochondrial function, and more. A wide range of enrichments amongst positive causal clusters have been hypothesized and/or demonstrated to contribute to CVD in SLE patients, including glucocorticoids, neutrophil cell death (NETosis) and degranulation, TNF-like weak inducer of apoptosis (TWEAK) signaling, canonical and alternative complement pathways, Th1 differentiation, lipid and lipoprotein metabolism among others.

Considering the drastically increased prevalence and mortality of CAD in SLE, the considerable portion of SLE-associated risk variants with negative causal effects on CAD was unexpected and suggested that numerous variants contributing to SLE have atheroprotective effects. Further SNP-to-gene mapping and detailed pathway analyses revealed that these variants are involved in various processes, predominantly related to oxidative stress and cholesterol homeostasis, whose atheroprotective effects have been found to be impaired in certain disease-related contexts, such as SLE. For example, the enzyme responsible for maintaining cholesterol homeostasis though lipoprotein lipase synthesis, cholesterol 27-hydroxylase, has been shown to be decreased in human monocytes and aortic endothelial cells of SLE patients, and is thought to impair the protective mechanism of efflux of cellular cholesterol (48). Cyp27a1 is the gene that encodes the cholesterol 27-hydroxylase and is an LXR target activated by oxysterols as well as a target of RXR and PPAR in human macrophages (49). LXR activation has additional proatherogenic and atheroprotective effects, as LXR activation in the liver promotes atherosclerosis via excess lipogenesis, whereas LXR activation in macrophages and dendritic cells has anti-inflammatory effects, linking lipid metabolism, immune cell function, and inflammation (50).

Our approach also has the advantage of identifying “actionable” points of therapeutic intervention with the potential to impact the inflammatory environment associated with CAD in SLE. This is especially important given that CAD risk in SLE cannot be fully accounted for by the increased prevalence of traditional atherosclerotic risk factors. SLE subjects therefore may derive particular benefit from treatments that mitigate inflammatory intermediates such as type I interferons with anifrolumab. Our findings also highlight additional putative targets, including PCSK9 involved in LDL receptor recycling. Inhibitors of PCSK9 activity, such as alirocumab and evolocumab are FDA approved to treat hyperlipidemia and may prove to be effective in controlling atherosclerosis in chronic inflammatory conditions (51). Finally, recent reports also support targeting oxidized LDL molecules (anti-oxLDL, orticumab) for the prevention of cardiovascular events in SLE (52).

SLE genetic association studies have been restricted in size and scope, yielding limited power and genomic coverage, especially considering the extensive heterogeneity and polygenicity of lupus. To maximize both power and scope, we used the largest genetic association study for SLE, which is limited to Immunochip SNPs, the largest SLE GWAS, as well as SLE-associated SNPs pooled from the Phenoscanner platform. However, most genetic association studies, including the multi-ancestral data used in this study, are heavily biased towards European ancestries. This is especially problematic given the increased CVD morbidity and mortality in SLE patients of African-ancestry (53) in addition to the ancestry-dependent disparities observed in both SLE and CAD. It is also of note, that certain risk factors leading to distinct phenotypic outcomes such as CAD are likely to be impacted by environmental factors that cannot be accounted for by genetics alone. This is important with respect to the higher disease burden observed in African ancestry patients, where barriers to treatment (such as delayed diagnosis and/or limited access to a specialist) may contribute to elevated mortality in this population and further underscores the importance of generating large datasets with diverse patient populations. In addition, the ability to map genetic variants to implicated genes is limited to known SNP-to-gene relationships included in Ensembl's variant effect predictor (VEP), Genotype-Tissue Expression (GTEx), and Human ACtive Enhancer to interpret Regulatory variants (HACER) databases. Although putative causal pathways associated with the HLA region are intriguing, mapping of the SNPs within the HLA region to genes is challenging because of the extensive LD across the region. Genes included in our PPI networks and clusters are protein-coding genes and interactions included in STRINGdb. This is a potential shortcoming of our pipeline especially considering the large number of non-coding genes implicated in our SNP-to-gene predictions in addition to the growing evidence highlighting the contributions of non-coding long RNAs and microRNAs in both SLE and CAD (54, 55). Ultimately, however, our robust SNP-to-gene mapping approach, which included multiple sources of information in combination with biologically informed clustering employing numerous sources of annotation, enabled comprehensive analysis of both small and large sets of genetic variants to specific pathways with excellent reproducibility.

In summary, we have employed various approaches to clearly identify shared genetic risk factors for SLE and CAD. In addition to showing a net positive causal effect of SLE on CAD by traditional MR, we mapped these variants to genes and pathways for interpretability and confirmed the implications of specific SLE-derived SNP-to-gene modules on CAD via a novel PPI-based MR approach. Both positive and negative causal effects were established by multiple approaches. Some of the SLE-derived risk involved pleiotropic pathways, only some of which have previously been assigned a role in SLE pathogenesis, and novel pathways not previously known to be involved in CAD and atherosclerosis pathogenesis. These results have provided new information about shared molecular pathways in SLE and CAD, as well as the genetic and molecular information to consider novel therapeutic interventions in these conditions.

### Method Details

Identification of SLE- and CAD-associated SNPs and overlap. SNPs associated with each disease were obtained from previous GWAS and Immunochip studies. For CAD, we used a comprehensive multi-ancestral meta-analysis of GWAS (32). For SLE, we included results of multiple GWAS and Immunochip studies to account for as many ancestries as possible (27-31). In total, 7,222 and 16,163 unique SNPs were significantly (p<10⁻⁶) associated with SLE and CAD, respectively, and were employed in these studies. A full list of the SNPs, chromosome locations, positions and sources used are detailed in Table 9A-9F.

Identification of SNP-predicted genes. Expression quantitative trait loci (eQTLs) were identified using GTEx (56) version 6.8 (GTEXportal.org) and mapped to their associated eQTL expression genes (E-Genes). To find SNPs in enhancers and promoters, and their associated transcription factors and downstream target genes (T-Genes), we queried the atlas of Human Active Enhancers to interpret Regulatory variants (57) (HACER, http://bioinfo.vanderbilt.edu/AE/HACER). To find SNPs in exons of protein-coding genes (C-Genes) and include proximal genes (P-Genes, within 5 kb), we queried the human Ensembl genome browser's variant effect predictor (58) (VEP, ensembl.org/info/docs/tools/vep, GRCh38.p12).

Stratified Linkage Disequilibrium Score Regression (S-LDSC). S-LDSC (33) was used to obtain gene-set specific disease-heritability estimates using GWAS summary statistics. Pre-processed summary statistics from SLE, CAD, CVD GWAS were obtained from Broad webpage (https://alkesgroup.broadinstitute.org/LDSCORE/all_sumstats/). Using the S-LDSC software provided on github (https://github.com/bulik/ldsc) and reference data on the Broad webpage (https://alkesgroup.broadinstitute.org/LDSCORE/), annotation and LD score files were generated for each SNP-predicted gene- and protein-set, separately. Using standard parameters, the “make_annot.py” and “ldsc.py” (with the “--l2” flag) scripts were first used to generate the gene-set-specific annotation and LD files, then the “ldsc.py” (with the “--h2-cts” flag) script was used to generate stratified heritability scores for each GWAS.

Network analysis and visualization. Protein-protein interaction (PPI) networks of SNP-predicted protein-coding genes were generated by STRING (59) (https://string-db.org, version 11.0b), and resulting networks were imported into Cytoscape (60) (version 3.6.1) for visualization and partitioned with MCODE via the clusterMaker2 (61) (version 1.2.1) plugin. Metastructures are based on PPI networks.

Functional gene set analysis. Predicted genes were examined using Biologically Informed Gene Clustering (BIG-C; version 4.4.). BIG-C is a custom functional clustering tool developed to annotate the biological meaning of large lists of genes and has been previously described (62-64) I-Scope is a custom clustering tool used to identify immune cell types in large gene datasets (65). The Ingenuity Pathway Analysis (IPA; https://www.qiagenbioinformatics.com) platform and EnrichR(66) (https://maayanlab.cloud/Enrichr/) web server provided additional molecular pathway enrichment analysis.

Mendelian Randomization (MR). MR was used to test for causal relationships between SLE and CAD using the MR-Base (45) (https://www.mrbase.org) TwoSampleMR (45) package in R (https://github.com/MRCIEU/TwoSampleMR). Various sets of SLE-associated genetic variants used as instrumental variables (IVs) and summary statistics for SLE-exposure were manually imported into R and summary statistics were carried out for MR-base compatibility using the ‘format data’ command. All effect sizes and standard errors were obtained from the exposure summary statistics used in each analysis, regardless of the study in which each IV was associated with the exposure. Given the availability of well-powered CAD/MI GWAS on MR-Base, IVs for CAD and MI were directly obtained from each exposure GWAS using the ‘extract instruments’ command for the bidirectional analyses. Data from the SLE and all CVD-related GWAS studies used in our MR analyses are publicly available and also accessible through the MR-Base software, which was used to obtain the outcome summary statistics via the ‘extract outcome data’ command. The ‘allele harmonization’ command was used to ensure the effect estimates of the exposure and outcome are based on matching alleles, excluding SNPs with completely mismatching alleles from the MR analysis or reversing the effect and non-effect alleles along with the effect estimates when applicable. Because of the allele harmonization step and because some SNPs are absent from the available summary statistics, a small proportion of SNPs used as IVs are absent from the final MR calculations. Up to sixteen individual MR methods were carried out through the TwoSampleMR package, including inverse variance weighted (IVW), simple mode, weighted mode, simple median, weighted median (WMedian), MR-Egger, MR-PRESSO (raw and outlier-corrected), MR-RAPS, and two sample maximum likelihood (ML). The ‘MR report’ function was used to generate a summary containing heterogeneity and directional pleiotropy tests and scatterplots (FIG. 4A-4D). MR-IVW and MR-Egger heterogeneity test results (Q-value) indicate whether significant heterogeneity was detected, which does not necessarily indicate biased causal estimates. MR-Egger intercept indicate whether significant directional horizontal pleiotropy was detected, which usually indicates biased causal estimates. For single-SNP MR, the ‘MR single-SNP’ function was also carried out using the Wald Ratio method. Full details of all MR results are included in Table 2A-2C.

Selection of valid, independent instrumental variables for traditional MR analysis. Traditional MR methods, such as MR-IVW, operate under assumptions for instrumental variable (IV) validity: 1) the relevance assumption, 2) the exclusion restriction criteria assumption, and 3) the independence assumption. To satisfy the relevance assumption, SNPs significantly (genome-wide significance p-value <5×10⁻⁸) associated with SLE (27-29, 67-81) were obtained from the Phenoscanner database (www.phenoscanner.medschl.cam.ac.uk) (82, 83) (Table 9A-9F). To satisfy the exclusion restriction criteria and independence assumptions, 89,336 SNPs weakly associated (p-value <1×10⁻⁵) with CVD and confounders including cholesterol, obesity, blood pressure, insulin resistance, smoking, age-related diseases, and many more, were excluded from being IVs for SLE-exposure (see Table 2A-2C for the full list of excluded traits). HLA-region SNPs were conservatively removed from MR analyses by excluding the short-arm of chromosome 6. Stringent LD clumping (37) was employed using the clump data (R²=0.001, 100 kb window, 1000G EA reference population) function to generate an independent set of 60 SLE-IVs harmonized for each GWAS.

PPI-based MR. SLE-associated variants from the Immunochip (31) and Phenoscanner database (82, 83) were linked to their most likely genes, and the genes used to generate PPI-informed gene clusters. The SLE-associated SNPs mapping to genes in each of PPI-based clusters were then extracted to “reverse engineer” subsets of SNPs that could be used separately as SLE-IVs for MR to independently estimate the causal effects of each PPI-informed SNP-to-Gene module on CAD. Up to sixteen MR methods were carried out for each SNP-to-gene module through the TwoSampleMR package.

In additional analyses (related to FIG. 13A-13B) using the combined Immunochip and Phenoscanner SNP dataset, filtering eliminated SNPs weakly associated (p-value <1×10⁻⁵) with CVD and confounders including cholesterol, obesity, blood pressure, insulin resistance, smoking, age-related diseases, and many more (Table 2A-2C). HLA-region SNPs were conservatively removed from MR analyses by excluding the short-arm of chromosome 6. Stringent LD clumping (37) was employed using the clump_data (R²=0.001, 100 kb window, 1000G EA reference population) function to generate an independent set of SLE-IVs. Various analyses were performed using independent, valid IVs derived from ‘All SNPs’, SNPs mapping to ‘Insignificant’ clusters, ‘Positive Tier 1’ clusters, ‘Positive Tier 1 and Tier 2’ clusters, ‘Positive MR-IVW p-value<0.00075’ clusters, ‘Negative MR-IVW p-value<0.00075’ clusters, ‘Negative Tier 1’ clusters, and ‘Negative Tier 1 and Tier 2’ clusters.

Monte Carlo Simulations for expected MR results using random sets of Immunochip-derived SNP-to-Gene modules. Monte Carlo Simulations were implemented and performed to estimate the false discovery rate with respect to significant PPI-based MR causal estimates. 120,026 Immunochip SNPs included in the SLE summary statistics were mapped to putative genes using the VEP, including regulatory effects, to generate an Immunochip SNP-to-Gene library with 67,211 unique SNPs mapping to 7,602 STRINGdb proteins. In each simulation, a random set of 3 to 152 SNP-predicted proteins were selected from the 7,602 proteins and used to extract up to 400 Immunochip SNPs. MR-IVW was then performed for SLE on CAD using harmonized, non-HLA SNPs (via removal of the entire short-arm of chromosome 6) from the random set of Immunochip SNPs as IVs. By using our Immunochip derived SNP-to-Gene dictionary for random selection of protein clusters and associated SNPs to generate random sets of IVs, our simulations account for both a high degree of LD and pleiotropy, especially considering the major influence of loci associated with diabetes in development of the Immunochip.

Drug candidate identification. Drug candidates were identified using LINCS (84), STITCH(85) (v5.0), IPA and literature mining. Each of the database tools includes either a programmatic method of matching existing therapeutics to their targets or else is a list of drugs and targets for achieving the same end.

TABLE 1
Canonical pathway and disease phenotype enrichments for network analysis of the 96
SNPs overlapping SLE and CAD. Heatmaps depict p-values from Fisher's exact test
that measures the significance of overlap between genes in each cluster and genes
within an annotation, red shading proportional to significance of each enrichment.

| | | P- | | | |
|---|---|---|---|---|---|
| Cluster | IPA canonical pathways | value | EnrichR | P-value | Dataset |
| 1 | Antigen Presentation | 1.00E−39 | Downstream TCR | 4.35E−17 | Reactome 2016 |
| | Pathway | | signaling | | |
| | B Cell Development | 2.51E−24 | MHC1 Causes AP | 5.80E−13 | Elsevier Pathway |
| | | | Failure in Cancer | | Collection |
| | | | Immune Escape | | |
| | PD-1, PD-L1 cancer | 6.31E−22 | Dendritic Cell Type | 5.54E−11 | HuBMAP ASCT |
| | immunotherapy | | 2 (Dc2) | | plus B augmented |
| | | | | | withRNAseq |
| | | | | | Coexpression |
| | Th1 and Th2 Activation | 2.51E−19 | Blood Vessel | 8.36E−11 | HuBMAP ASCT |
| | Pathway | | Endothelial Cell | | plus B augmented |
| | | | | | with RNAseq |
| | | | | | Coexpression |
| | MSP-RON Signaling In | 5.01E−17 | Sudden cardiac | 5.68E−06 | Human |
| | Macrophages | | death | | Phenotype |
| | | | | | Ontology |
| 2 | Complement System | 3.16E−03 | Activation of C3 | 4.50E−07 | Reactome 2016 |
| | | | and C5 Homo | | |
| | | | sapiens | | |
| | Acute Phase Response | 1.55E−02 | Lectin Induced | 2.60E−03 | BioCarta 2016 |
| | Signaling | | Complement | | |
| | | | Pathway Homo | | |
| | | | sapiens | | |
| 3 | FXR/RXR Activation | 3.55E−04 | Lupus | 4.43E−06 | dbGAP |
| | | | Erythematosus, | | |
| | | | Systemic | | |
| | Clathrin-mediated | 1.23E−03 | Behcet Syndrome | 2.66E−03 | dbGAP |
| | Endocytosis | | | | |
| | Atherosclerosis Signaling | 8.91E−03 | Metabolic pathway | 2.06E−02 | WikiPathway |
| | | | of LDL, HDL and | | 2021 Human |
| | | | TG, including | | |
| | | | diseases | | |
| | IL-12 Signaling and | 9.77E−03 | PPAR-alpha | 3.33E−02 | WikiPathway |
| | Production in Macrophages | | pathway | | 2021 Human |
| | Production of NO and ROS | 1.86E−02 | Phospholipids | 4.08E−02 | dbGAP |
| | in Macrophages | | | | |
| 4 | Triacylglycerol | 2.51E−02 | BAT3 complex | 2.75E−10 | Jensen |
| | Degradation | | | | Compartments |
| | Caveolar-mediated | 3.72E−02 | ER membrane | 3.19E−09 | Jensen |
| | Endocytosis | | insertion complex | | Compartments |
| 5 | ESC Differentiation into | 4.27E−03 | Behcet Syndrome | 1.58E−18 | PhenGenI |
| | Cardiac Lineages | | | | Association 2021 |
| | tRNA Charging | 1.62E−02 | Immune System | 7.02E−08 | DisGeNET |
| | | | Diseases | | |
| | Role of OCT4 in | 1.95E−02 | Glomerulonephritis, | 2.93E−07 | PhenGenI |
| | Mammalian ESC | | Membranous | | Association 2021 |
| | Pluripotency | | | | |
| | Transcriptional Regulatory | 2.29E−02 | let-7 inhibition of | 3.00E−03 | WikiPathway |
| | Network in ESCs | | ES cell | | 2021 Human |
| | | | reprogramming | | |
| | Crosstalk between DC and | 3.80E−02 | combined | 1.84E−02 | Clin Var 2019 |
| | NK Cells | | OXPHOS | | |
| | | | deficiency | | |
| 6 | Fatty Acid α-oxidation | 7.59E−03 | Mean arterial | 5.68E−10 | GWAS Catalog |
| | | | pressure | | 2019 |
| | Role of JAK2 in Hormone- | 1.29E−02 | Coronary heart | 2.52E−08 | GWAS Catalog |
| | like Cytokine Signaling | | disease | | 2019 |
| | Noradrenaline and | 1.32E−02 | Coronary Disease | 1.02E−05 | PhenGenI |
| | Adrenaline Degradation | | | | Association 2021 |
| | Oxidative Ethanol | 2.09E−02 | Myocardial | 4.94E−03 | Human |
| | Degradation III | | infarction | | Phenotype |
| | | | | | Ontology |
| | p38 MAPK Signaling | 4.37E−02 | negative regulation | 2.70E−03 | GO Biological |
| | | | of chemokine- | | Process 2021 |
| | | | mediated signaling | | |
| 7 | Complement System | 1.26E−02 | 21-hydroxylase | 4.89E−05 | Rare Diseases |
| | | | deficiency | | GeneRIF Gene |
| | | | | | Lists |
| | Triacylglycerol | 1.58E−02 | Activation of C3 | 2.40E−03 | BioPlanet 2019 |
| | Biosynthesis | | and C5 | | |
| | | 1.66E−02 | ChREBP activates | 3.20E−03 | BioPlanet 2019 |
| | | | metabolic gene | | |
| | | | expression | | |
| | FXR/RXR Activation | 4.17E−02 | Focal Adhesion- | 6.03E−03 | WikiPathway |
| | | | PI3K-Akt-mTOR- | | 2021 Human |
| | | | signaling | | |
| | PI3K Signaling in B | 4.79E−02 | Complement | 1.75E−02 | Rare Diseases |
| | Lymphocytes | | component | | GeneRIF Gene |
| | | | deficiency | | Lists |
| 8 | Role of BRCA1 in DNA | 1.35E−02 | Fanconi anemia | 4.28E−05 | KEGG 2021 |
| | Damage Response | | pathway | | Human |
| | Hereditary Breast Cancer | 2.40E−02 | interstrand cross- | 4.44E−05 | GO Biological |
| | Signaling | | link repair | | Process 2021 |
| 9 | CD27 Signaling in | 7.24E−03 | negative regulation | 9.00E−04 | GO Biological |
| | Lymphocytes | | of SMAD protein | | Process 2021 |
| | | | signal transduction | | |
| | NGF Signaling | 1.48E−02 | mitogen-activated | 1.65E−03 | GO Molecular |
| | | | protein kinase | | Function 2021 |
| | | | kinase kinase | | |
| | | | binding | | |
| | IL-15 Production | 1.55E−02 | Tandem pore | 1.80E−03 | BioPlanet 2019 |
| | | | domain potassium | | |
| | | | channels | | |
| | Reelin Signaling in | 1.58E−02 | Endocarditis | 2.70E−03 | Jensen |
| | Neurons | | | | DISEASES |
| | Production of NO and ROS | 2.40E−02 | Brain ischemia | 5.24E−03 | Jensen |
| | in Macrophages | | | | DISEASES |
| 10 | BAG2 Signaling Pathway | 1.26E−05 | chaperone cofactor- | 1.95E−09 | GO Biological |
| | | | dependent protein | | Process 2021 |
| | | | refolding | | |
| | Unfolded protein response | 1.45E−05 | cellular response to | 3.72E−09 | GO Biological |
| | | | topologically | | Process 2021 |
| | | | incorrect protein | | |
| | PKR in IFN Induction and | 3.31E−05 | regulation of | 2.00E−07 | GO Biological |
| | Antiviral Response | | cellular response to | | Process 2021 |
| | | | stress | | |
| | eNOS Signaling | 4.47E−05 | Carotid artery | 4.14E−06 | DisGeNET |
| | | | occlusion | | |
| | Aldosterone Signaling in | 4.79E−05 | Hypoxia and p53 in | 3.15E−03 | BioCarta 2016 |
| | Epith. Cells | | the Cardiovascular | | |
| | | | system H. sapiens | | |
| 11 | Glucocorticoid Receptor | 1.12E−03 | Lupus | 6.33E−06 | dbGAP |
| | Signaling | | Erythematosus, | | |
| | | | Systemic | | |
| | Oxidized GTP and dGTP | 1.51E−03 | Coronary artery | 8.52E−03 | GWAS Catalog |
| | Detoxification | | disease or ischemic | | 2019 |
| | | | stroke | | |
| | Aryl Hydrocarbon | 1.58E−03 | Lipoproteins, LDL | 1.61E−02 | dbGAP |
| | Receptor Signaling | | | | |
| | RAR Activation | 2.40E−03 | Behcet Syndrome | 2.62E−02 | dbGAP |
| | Retinoic acid Mediated | 2.29E−02 | Coronary Disease | 3.63E−02 | dbGAP |
| | Apoptosis | | | | |
| 12 | Crosstalk between DC and | 1.51E−02 | Behcet Syndrome | 4.69E−09 | PhenGenI |
| | NK Cells | | | | Association 2021 |
| | Natural killer cell signaling | 3.31E−02 | Systemic Lupus | 3.47E−07 | PhenGenI |
| | | | Erythematosus | | Association 2021 |

Table 2: List of all included SLE and excluded CVD/confounder-associated traits from the Phenoscanner database for use as SLE-IVs in MR analyses.

TABLE 2A
Summary of SLE included traits

| Summary of SLE included traits: | References (#) |
|---|---|
| Systemic lupus erythematosus | 27, 28, 29, 68, 69, 70, 71, 72, 73, 74, 75, |
| Lupus erythematosus systemic | 76, 77, 78, 79, 80, 81, 86 & UK Biobank |
| Systemic lupus erythematosus SLE | |
| Systemic lupus erythematosus SLE females | |

TABLE 2B
Summary of CVD/confounder excluded traits

Adiposity; Age related disease endophenotypes; Age related diseases and mortality; Age related
diseases mortality and associated endophenotypes; Bilirubin levels in extreme obesity; Blood
pressure; Blood pressure traits multi trait analysis; C reactive protein levels or HDL cholesterol
levels pleiotropy; C reactive protein levels or LDL cholesterol levels pleiotropy; C reactive protein
levels or triglyceride levels pleiotropy; Cardiovascular disease in hypertension ACE inhibitor
interaction; Cardiovascular disease in hypertension calcium channel blocker interaction;
Cardiovascular disease risk factors; Childhood obesity; Cholesterol and Triglycerides; Coronary
artery disease; Coronary artery disease or ischemic stroke; Coronary artery disease or large artery
stroke; Coronary disease; Coronary heart disease; Coronary heart disease or coronary artery
disease; Coronary heart disease with local African versus European ancestry; Diabetes diagnosed
by doctor; Diabetes in response to antihypertensive drug treatment treatment strategy interaction;
Diabetes incident; Diabetes mellitus; Diabetes mellitus type 1; Diabetes mellitus type 2; Diarrhoea
in darapladib treated cardiovascular disease time to event; Diastolic blood pressure response to
hydrochlorothiazide in hypertension; Differential response to pravastatin therapy for coronary heart
disease; Essential hypertension; Glomerular filtration rate in diabetics creatinine; HDL cholesterol;
HDL Cholesterol Triglycerides HDLC TG; HDL Cholesterol in HIV infection; HDL cholesterol
large lipoprotein fraction concentration; HDL cholesterol large lipoprotein fraction concentration in
fasting sample; HDL cholesterol levels; HDL cholesterol lipoprotein fraction concentration; HDL
cholesterol lipoprotein fraction concentration by NMR; HDL cholesterol lipoprotein fraction
concentration by NMR in fasting sample; HDL cholesterol lipoprotein fraction concentration in
fasting sample; HDL cholesterol mean particle size lipoprotein fraction concentration; HDL
cholesterol mean size lipoprotein fraction concentration; HDL cholesterol mean size lipoprotein
fraction concentration in fasting sample; HDL cholesterol medium lipoprotein fraction
concentration; HDL cholesterol medium lipoprotein fraction concentration in fasting sample; HDL
cholesterol small lipoprotein fraction concentration; HDL cholesterol small lipoprotein fraction
concentration in fasting sample; HDL cholesterol total lipoprotein fraction concentration; HDL
cholesterol total lipoprotein fraction concentration in fasting sample; Heart diseases; Hepatic lipid
content in extreme obesity; High density lipoprotein; High density lipoprotein cholesterol levels;
Hypertension; Hypertension early onset hypertension; Hypertension risk in short sleep duration;
Hypertension with local African versus European ancestry; Hypertriglyceridemia; Incident
coronary heart disease; Incident coronary heart disease excluding revascularization procedures;
Incident myocardial infarction; Ischemic stroke large artery atherosclerosis; Large artery stroke;
Large vessel stroke; LDL cholesterol; LDL cholesterol levels; LDL oxidized; Lipid levels in
hepatitis C treatment; Lipid traits; Lipoprotein associated phospholipase A2 activity change in
response to darapladib treatment in cardiovascular disease; Lipoprotein phospholipase A2 activity
in cardiovascular disease; log Urinary albumin creatinine ratio in diabetics; Low density
lipoprotein; Low density lipoprotein cholesterol; Low high density lipoprotein cholesterol levels;
Major coronary event in cardiovascular disease time to event darapladib treatment interaction;
Major coronary event in darapladib treated cardiovascular disease time to event; Major coronary
event in placebo treated cardiovascular disease time to event; Mean arterial pressure; Mean arterial
pressure alcohol consumption interaction; Mean arterial pressure long term average; Medication
adherence in chronic diseases; Metabolic traits; Metabolite levels; Moderate or severe diarrhoea in
darapladib treated cardiovascular disease time to event; Myocardial infarction; Myocardial
infarction early age of onset; Myocardial infarction early onset; Myocardial infarction in
cardiovascular disease time to event darapladib treatment interaction; Myocardial infarction in
coronary artery disease; Myocardial infarction in darapladib treated cardiovascular disease time to
event; Myocardial infarction in hypertension calcium channel blocker interaction; Myocardial
infarction in patients with coronary artery disease by angiography; Myocardial infarction male;
Myocardial infarction sudden cardiac arrest in patients with coronary artery disease; Myocardial
infarction sudden cardiac death; Myocardial infarction sudden cardiac death in ST elevation
myocardial infarction with ventricular fibrillation; Myocardial infarction women; Obesity; Obesity
and osteoporosis; Obesity body mass index; Obesity body mass index 30 kgm2; Obesity body
mass index females; Obesity body mass index males; Obesity class 1; Obesity class 2; Obesity
class 3; Obesity early onset extreme; Obesity extreme; Obesity in adult survivors of childhood
cancer exposed to cranial radiation; Obesity in adult survivors of childhood cancer not exposed to
cranial radiation; Obesity related traits; Perioperative myocardial infarction in coronary artery
bypass surgery; Response to angiotensin II receptor blocker therapy; Response to angiotensin II
receptor blocker therapy opposite direction w diuretic therapy; Response to statins LDL cholesterol
change; Self-reported diabetes; Self-reported hypertension; Smoking cessation; Smoking cessation
in chronic obstructive pulmonary disease; Statin response difference HDL; Statin response
difference LDL; Statin response difference triglycerides; Systolic blood pressure response to
hydrochlorothiazide in hypertension; Triglyceride levels; Triglycerides; Triglycerides Blood
Pressure TG BP; Type 2 diabetes young onset and obesity; Urinary albumin to creatinine ratio in
diabetes; Waist Circumference Triglycerides WC TG;

TABLE 2C
Excluded CVD traits. Listed by: Excluded Trait | Study | Pubmed ID | ancestry Year.

Adiposity | Lindgren CM | 19557161 | European | 2009; Adiposity | Kilpelainen TO | 21706003 |
Mixed | 2011; Adiposity | Liu XG | 19268274 | Mixed | 2009; Adiposity | Plourde M | 23505185 |
European | 2012; Age related disease endophenotypes | He L | 27790247 | European | 2016; Age
related diseases and mortality | He L | 27790247 | European | 2016; Age related diseases mortality
and associated endophenotypes | He L | 27790247 | European | 2016; Bilirubin levels in extreme
obesity | DiStefano JK | 25246029 | European | 2014; Blood pressure | Simino J | 24954895 | Mixed
| 2014; Blood pressure | Kelly TN | 24001895 | East Asian | 2013; Blood pressure traits multi trait
analysis | Liang J | 28498854 | Mixed | 2017; C reactive protein levels or HDL cholesterol levels
pleiotropy | Ligthart S | 27286809 | Unspecified | 2016; C reactive protein levels or LDL cholesterol
levels pleiotropy | Ligthart S | 27286809 | Unspecified | 2016; C reactive protein levels or
triglyceride levels pleiotropy | Ligthart S | 27286809 | Unspecified | 2016; Cardiovascular disease in
hypertension ACE inhibitor interaction | Bis JC | 26516778 | Mixed | 2015; Cardiovascular disease
in hypertension calcium channel blocker interaction | Bis JC | 26516778 | Mixed | 2015;
Cardiovascular disease risk factors | Middelberg RP | 21943158 | European | 2011; Childhood
obesity | EGGC | 22484627 | European | 2012; Cholesterol and Triglycerides | Surakka I | 23031429
| European | 2012; Coronary artery disease | van der Harst P | 29212778 | Mixed | 2018; Coronary
artery disease | CARDIOGRAMplusC4D | 26343387 | Mixed | 2015; Coronary artery disease |
Nelson CP | 28714975 | Mixed | 2017; Coronary artery disease | CARDIOGRAMplusC4D |
23202125 | Mixed | 2013; Coronary artery disease | CARDIoGRAM | 21378990 | European | 2011; |
Coronary artery disease | C4D | 21378988 | Mixed | 2011; Coronary artery disease | Howson_JMM
28530674 | Mixed | 2017; Coronary artery disease | Howson_JMM | 28530674 | European | 2017;
Coronary artery disease | Dichgans M | 24262325 | Unspecified | 2013; Coronary artery disease |
Reilly MP | 21239051 | European | 2011; Coronary artery disease or ischemic stroke | Dichgans M |
24262325 | Unspecified | 2013; Coronary artery disease or large artery stroke | Dichgans M |
24262325 | Unspecified | 2013; Coronary disease | Lettre G | 21347282 | African | 2011; Coronary
disease | Samani NJ | 17634449 | European | 2007; Coronary disease | WTCCC | 17554300 |
European | 2007; Coronary disease | C4D | 21378988 | Mixed | 2011; Coronary heart disease |
Lettre G | 21347282 | African | 2011; Coronary heart disease | Richards JB | 20011104 | European
2009; Coronary heart disease | McPherson | 17478681 | Mixed | 2007; Coronary heart disease |
CARDIoGRAM | 21378990 | European | 2011; Coronary heart disease | C4D | 21378988 | Mixed |
2011; Coronary heart disease | Mehta NN | 21846871 | Mixed | 2011; Coronary heart disease |
Samani NJ | 17634449 | European | 2007; Coronary heart disease | Davies RW | 22319020 |
European | 2012; Coronary heart disease | Domarkiene I | 24778558 | European | 2013; Coronary
heart disease | WTCCC | 17554300 | European | 2007; Coronary heart disease | Lu X | 22751097 |
East Asian | 2012; Coronary heart disease | Erdmann J | 19198612 | European | 2009; Coronary
heart disease | Hager J | 22745674 | Arab | 2012; Coronary heart disease | Takeuchi F | 21971053 |
East Asian | 2011; Coronary heart disease | Wild PS | 21606135 | European | 2011; Coronary heart
disease | Tregouet DA | 19198611 | European | 2009; Coronary heart disease | Lee JY | 23364394 |
East Asian | 2013; Coronary heart disease | Erdmann J | 21088011 | European | 2010; Coronary
heart disease or coronary artery disease | Grallert H | 22003152 | European | 2011; Coronary heart
disease with local African versus European ancestry | Lettre G | 21347282 | African | 2011;
Diabetes diagnosed by doctor | Neale B | UKBB | European | 2017; Diabetes in response to
antihypertensive drug treatment treatment strategy interaction | Chang SW | 27670767 | Mixed |
2016; Diabetes incident | Meigs JB | 17903298 | European | 2007; Diabetes mellitus | Meigs JB |
17903298 | European | 2007; Diabetes mellitus type 1 | Barrett JC | 19430480 | European | 2009;
Diabetes mellitus type 1 | Grant SF | 18840781 | European | 2008; Diabetes mellitus type 1 | Cooper
JD | 18978792 | European | 2008; Diabetes mellitus type 1 | WTCCC | 17554300 | European | 2007;
Diabetes mellitus type 1 | Todd JA | 17554260 | European | 2007; Diabetes mellitus type 1 |
Hakonarson H | 17632545 | European | 2007; Diabetes mellitus type 1 | Plagnol V | 21829393 |
Mixed | 2011; Diabetes mellitus type 1 | Bradfield JP | 21980299 | European | 2011; Diabetes
mellitus type 1 | Huang J | 22293688 | European | 2012; Diabetes mellitus type 1 | Hakonarson H |
18198356 | European | 2008; Diabetes mellitus type 1 | Wallace C | 19966805 | European | 2009;
Diabetes mellitus type 2 | Aberg K | 19875103 | Mixed | 2009; Diabetes mellitus type 2 | Murea M |
21546767 | African | 2011; Diabetes mellitus type 2 | Palmer ND | 22238593 | African | 2012;
Diabetes mellitus type 2 | MAGIC | 21873549 | European | 2011; Diabetes mellitus type 2 | Scott LJ
| 17463248 | European | 2007; Diabetes mellitus type 2 | Zeggini E | 18372903 | European | 2008;
Diabetes mellitus type 2 | Sim X | 21490949 | Mixed | 2011; Diabetes mellitus type 2 | Voight BF |
20581827 | European | 2010; Diabetes mellitus type 2 | Salonen JT | 17668382 | European | 2007;
Diabetes mellitus type 2 | Qi L | 20418489 | European | 2010; Diabetes mellitus type 2 | Kooner JS |
21874001 | South Asian | 2011; Diabetes mellitus type 2 | Rung J | 19734900 | European | 2009;
Diabetes mellitus type 2 | Saxena R | 17463246 | European | 2007; Diabetes mellitus type 2 |
Zeggini E | 17463249 | European | 2007; Diabetes mellitus type 2 | WTCCC | 17554300 | European
| 2007; Diabetes mellitus type 2 | Cho YS | 22158537 | Mixed | 2011; Diabetes mellitus type 2 |
Takeuchi F | 19401414 | East Asian | 2009; Diabetes mellitus type 2 | Unoki H | 18711366 | Mixed |
2008; Diabetes mellitus type 2 | Parra EJ | 21573907 | Hispanic | 2011; Diabetes mellitus type 2 |
Imamura M | 22456796 | East Asian | 2012; Diabetes mellitus type 2 | Timpson NJ | 19056611 |
European | 2008; Diabetes mellitus type 2 | Steinthorsdottir V | 17460697 | European | 2007;
Diabetes mellitus type 2 | Sladek R | 17293876 | European | 2007; Diabetes mellitus type 2 | Tsai FJ
| 20174558 | East Asian | 2010; Diabetes mellitus type 2 | Below JE | 21647700 | Hispanic | 2011;
Diabetes mellitus type 2 | Huang J | 22293688 | European | 2012; Diabetes mellitus type 2 | Shu XO
| 20862305 | Mixed | 2010; Diabetes mellitus type 2 | Zabaneh D | 20694148 | South Asian | 2010;
Diabetes mellitus type 2 | Kho AN | 22101970 | Mixed | 2011; Diabetes mellitus type 2 | Yasuda K |
18711367 | Mixed | 2008; Diabetes mellitus type 2 | Cui B | 21799836 | East Asian | 2011; Diabetes
mellitus type 2 | Yamauchi T | 20818381 | Mixed | 2010; Diabetes mellitus type 2 | Wallace C |
19966805 | European | 2009; Diarrhoea in darapladib treated cardiovascular disease time to event |
Yeo A | 28753643 | Mixed | 2017; Diastolic blood pressure response to hydrochlorothiazide in
hypertension | Salvi E | 27802415 | Mixed | 2016; Differential response to pravastatin therapy for
coronary heart disease | Shiffman D | 22666496 | Mixed | 2012; Essential hypertension | Neale B |
UKBB | European | 2017; Essential hypertension | Salvi | 22184326 | European | 2011; Glomerular
filtration rate in diabetics creatinine | CKDGen | 26831199 | European | 2016; HDL cholesterol |
Nagy R | 28270201 | European | 2017; HDL cholesterol | GLGC | 24097068 | European | 2013;
HDL cholesterol | Surakka I | 25961943 | European | 2015; HDL cholesterol | GLGC | 20686565 |
Mixed | 2010; HDL cholesterol | Lettre G | 21347282 | African | 2011; HDL cholesterol | Willer CJ |
18193043 | European | 2008; HDL cholesterol | Kathiresan S | 18193044 | Mixed | 2008; HDL
cholesterol | Kathiresan S | 19060906 | European | 2008; HDL cholesterol | Waterworth DM |
20864672 | Mixed | 2010; HDL cholesterol | Aulchenko YS | 19060911 | European | 2008; HDL
cholesterol | Below JE | 26780889 | Hispanic | 2016; HDL cholesterol | Coram MA | 23726366 |
Mixed | 2013; HDL cholesterol | van Leeuwen EM | 27036123 | Mixed | 2016; HDL cholesterol
Weissglas Volkov D | 23505323 | Hispanic | 2013; HDL cholesterol | Heid IM | 20031538
European | 2008; HDL cholesterol | Lu X | 26582766 | East Asian | 2015; HDL cholesterol | Kim YJ
| 21909109 | East Asian | 2011; HDL cholesterol | Sabatti C | 19060910 | European | 2008; HDL
cholesterol | Zhu Y | 28371326 | East Asian | 2017; HDL cholesterol | Ko A | 24886709 | Hispanic |
2014; HDL cholesterol | Hiura Y | 19359809 | East Asian | 2009; HDL cholesterol | Ridker PM |
20031564 | European | 2009; HDL Cholesterol Triglycerides HDLC TG | Kraja AT | 21386085 |
European | 2011; HDL Cholesterol in HIV infection | Moore CB | 25884002 | Unspecified | 2015;
HDL cholesterol large lipoprotein fraction concentration | Chasman DI | 19936222 | European |
2009; HDL cholesterol large lipoprotein fraction concentration in fasting sample | Chasman DI |
19936222 | European | 2009; HDL cholesterol levels | Spracklen CN | 28334899 | East Asian |
2017; HDL cholesterol levels | Kurano M | 26763881 | Asain | 2016; HDL cholesterol lipoprotein
fraction concentration | Chasman DI | 19936222 | European | 2009; HDL cholesterol lipoprotein
fraction concentration by NMR | Chasman DI | 19936222 | European | 2009; HDL cholesterol
lipoprotein fraction concentration by NMR in fasting sample | Chasman DI | 19936222 | European |
2009; HDL cholesterol lipoprotein fraction concentration in fasting sample | Chasman DI |
19936222 | European | 2009; HDL cholesterol mean particle size lipoprotein fraction concentration
| Chasman DI | 19936222 | European | 2009; HDL cholesterol mean size lipoprotein fraction
concentration | Chasman DI | 19936222 | European | 2009; HDL cholesterol mean size lipoprotein
fraction concentration in fasting sample | Chasman DI | 19936222 | European | 2009; HDL
cholesterol medium lipoprotein fraction concentration | Chasman DI | 19936222 | European | 2009;
HDL cholesterol medium lipoprotein fraction concentration in fasting sample | Chasman DI |
19936222 | European | 2009; HDL cholesterol small lipoprotein fraction concentration | Chasman
DI | 19936222 | European | 2009; HDL cholesterol small lipoprotein fraction concentration in
fasting sample | Chasman DI | 19936222 | European | 2009; HDL cholesterol total lipoprotein
fraction concentration | Chasman DI | 19936222 | European | 2009; HDL cholesterol total
lipoprotein fraction concentration in fasting sample | Chasman DI | 19936222 | European | 2009;
Heart diseases | Larson MG | 17903304 | European | 2007; Hepatic lipid content in extreme obesity
| DiStefano JK | 25246029 | European | 2014; High density lipoprotein | Prins B | 28887542 |
European | 2017; High density lipoprotein | GLGC | 24097068 | European | 2013; High density
lipoprotein | GLGC | 20686565 | Mixed | 2010; High density lipoprotein | Spracklen CN | 28334899
| East Asian | 2017; High density lipoprotein cholesterol levels | Southam L | 28548082 | Mixed |
2017; Hypertension | BPExome | 27618447 | Mixed | 2016; Hypertension | Kim SJ | 22322875 |
East Asian | 2012; Hypertension | ICBP | 21909115 | European | 2011; Hypertension | WTCCC |
17554300 | European | 2007; Hypertension | Guo Y | 22384028 | East Asian | 2012; Hypertension |
Adeyemo A | 19609347 | African | 2009; Hypertension | Arnett DK | 19454037 | Mixed | 2009;
Hypertension | Levy D | 19430479 | European | 2009; Hypertension | Padmanabhan S | 21082022 |
European | 2010; Hypertension | Org E | 19304780 | European | 2009; Hypertension | Kato N |
21572416 | East Asian | 2011; Hypertension | Lettre G | 21347282 | African | 2011; Hypertension |
Johnson | 22100073 | European | 2011; Hypertension | Newton Cheh C | 19430483 | Mixed | 2009;
Hypertension | Ganesh SK | 19862010 | European | 2009; Hypertension | Yamada | 21211798 | East
Asian | 2010; Hypertension | Lu X | 22751097 | East Asian | 2012; Hypertension | Saxena R |
17463246 | European | 2007; Hypertension | Lu X | 25249183 | East Asian | 2014; Hypertension |
Park YM | 28273873 | East Asian | 2017; Hypertension | Liang J | 28498854 | Mixed | 2017;
Hypertension | Liu X | 27480026 | East Asian | 2016; Hypertension early onset hypertension | Yang
| 22479346 | Mixed | 2012; Hypertension risk in short sleep duration | Kim SJ | 22322875 | East
Asian | 2012; Hypertension with local African versus European ancestry | Lettre G | 21347282 |
African | 2011; Hypertriglyceridemia | Weissglas Volkov D | 23505323 | Hispanic | 2013; Incident
coronary heart disease | Jensen | 21880673 | European | 2011; Incident coronary heart disease |
Barbalic | 21829389 | African | 2011; Incident coronary heart disease | Dehghan A | 26950853 |
Mixed | 2016; Incident coronary heart disease excluding revascularization procedures | Barbalic |
21829389 | African | 2011; Incident myocardial infarction | Dehghan A | 26950853 | Mixed | 2016;
Ischemic stroke large artery atherosclerosis | Cheng YC | 26732560 | Mixed | 2016; Ischemic stroke
large artery atherosclerosis | Pulit SL | 26708676 | Mixed | 2015; Large artery stroke | Dichgans M |
24262325 | Unspecified | 2013; Large artery stroke | Traylor M | 27997041 | European | 2016; Large
vessel stroke | ISGS | 17434096 | European | 2007; LDL cholesterol | Surakka I | 25961943 |
European | 2015; LDL cholesterol | GLGC | 20686565 | Mixed | 2010; LDL cholesterol | GLGC |
24097068 | European | 2013; LDL cholesterol | Willer CJ | 18193043 | European | 2008; LDL
cholesterol | Kathiresan S | 19060906 | European | 2008; LDL cholesterol | Waterworth DM |
20864672 | Mixed | 2010; LDL cholesterol | Lu X | 26582766 | East Asian | 2015; LDL cholesterol |
Kathiresan S | 18193044 | Mixed | 2008; LDL cholesterol | Aulchenko YS | 19060911 | European |
2008; LDL cholesterol | Lettre G | 21347282 | African | 2011; LDL cholesterol | Below JE |
26780889 | Hispanic | 2016; LDL cholesterol | Sabatti C | 19060910 | European | 2008; LDL
cholesterol | Wallace C | 18179892 | European | 2008; LDL cholesterol | Sandhu MS | 18262040 |
European | 2008; LDL cholesterol | Burkhardt R | 18802019 | Mixed | 2008; LDL cholesterol |
Rasmussen Torvik LJ | 23067351 | African | 2012; LDL cholesterol levels | Spracklen CN |
28334899 | East Asian | 2017; LDL cholesterol levels | Kurano M | 26763881 | Asain | 2016; LDL
oxidized | Makela KM | 23247145 | European | 2012; Lipid levels in hepatitis C treatment | Clark PJ
| 22497812 | Mixed | 2012; Lipid traits | Keller M | 24023261 | European | 2013; Lipid traits | Wu Y
| 24023260 | Filipino | 2013; Lipoprotein associated phospholipase A2 activity change in response
to darapladib treatment in cardiovascular disease | Yeo A | 28753643 | Mixed | 2017; Lipoprotein
phospholipase A2 activity in cardiovascular disease | Yeo A | 28753643 | Mixed | 2017; log Urinary
albumin creatinine ratio in diabetics | CKDGen | 26631737 | European | 2016; Low density
lipoprotein | GLGC | 24097068 | European | 2013; Low density lipoprotein | GLGC | 20686565 |
Mixed | 2010; Low density lipoprotein | Prins B | 28887542 | European | 2017; Low density
lipoprotein | Spracklen CN | 28334899 | East Asian | 2017; Low density lipoprotein cholesterol |
Southam L | 28548082 | Mixed | 2017; Low high density lipoprotein cholesterol levels | Wakil SM |
26879886 | Arab | 2016; Major coronary event in cardiovascular disease time to event darapladib
treatment interaction | Yeo A | 28753643 | Mixed | 2017; Major coronary event in darapladib
treated cardiovascular disease time to event | Yeo A | 28753643 | Mixed | 2017; Major coronary
event in placebo treated cardiovascular disease time to event | Yeo A | 28753643 | Mixed | 2017;
Mean arterial pressure | Johnson | 22100073 | European | 2011; Mean arterial pressure | Wain LV |
21909110 | European | 2011; Mean arterial pressure | de Las Fuentes | 22510845 | European | 2012;
Mean arterial pressure | Guo Y | 22384028 | East Asian | 2012; Mean arterial pressure | Levy D |
17903302 | European | 2007; Mean arterial pressure | Kato N | 26390057 | Mixed | 2015; Mean
arterial pressure alcohol consumption interaction | Simino J | 24376456 | Unspecified | 2013; Mean
arterial pressure long term average | Li C | 28348047 | Mixed | 2017; Medication adherence in
chronic diseases | Seo I | 25317111 | East Asian | 2014; Metabolic traits | Sabatti C | 19060910 |
European | 2008; Metabolite levels | Kim YJ | 21909109 | East Asian | 2011; Moderate or severe
diarrhoea in darapladib treated cardiovascular disease time to event | Yeo A | 28753643 | Mixed |
2017; Myocardial infarction | CARDIOGRAMplusC4D | 26343387 | Mixed | 2015; Myocardial
infarction | Kathiresan S | 19198609 | European | 2009; Myocardial infarction | Aoki A | 21107343 |
East Asian | 2010; Myocardial infarction | Helgadottir A | 17478679 | European | 2007; Myocardial
infarction | Yamada | 21211798 | East Asian | 2010; Myocardial infarction | CARDIoGRAM |
21378990 | European | 2011; Myocardial infarction | Erdmann J | 21088011 | European | 2010;
Myocardial infarction | Ebana | 17211523 | East Asian | 2007; Myocardial infarction | Ozaki |
12426569 | East Asian | 2002; Myocardial infarction | Takeuchi F | 21971053 | East Asian | 2011;
Myocardial infarction | ODonnell CJ | 22144573 | European | 2011; Myocardial infarction |
Gudbjartsson DF | 19198610 | Mixed | 2009; Myocardial infarction | Soranzo N | 19820697 |
European | 2009; Myocardial infarction | GUGC | 23263486 | European | 2012; Myocardial
infarction | Ishii | 17066261 | East Asian | 2006; Myocardial infarction | Hirokawa M | 24916648 |
East Asian | 2014; Myocardial infarction | Wakil SM | 26708285 | Arab | 2016; Myocardial
infarction early age of onset | Kathiresan S | 19198609 | European | 2009; Myocardial infarction
early onset | Kathiresan S | 19198609 | European | 2009; Myocardial infarction in cardiovascular
disease time to event darapladib treatment interaction | Yeo A | 28753643 | Mixed | 2017;
Myocardial infarction in coronary artery disease | Reilly MP | 21239051 | European | 2011;
Myocardial infarction in darapladib treated cardiovascular disease time to event | Yeo A | 28753643
| Mixed | 2017; Myocardial infarction in hypertension calcium channel blocker interaction | Bis JC |
26516778 | Mixed | 2015; Myocardial infarction in patients with coronary artery disease by
angiography | Reilly MP | 21239051 | European | 2011; Myocardial infarction male | Yamada |
21211798 | East Asian | 2010; Myocardial infarction sudden cardiac arrest in patients with coronary
artery disease | Aouizerat BE | 21658281 | European | 2011; Myocardial infarction sudden cardiac
death | Arking DE | 21738491 | European | 2011; Myocardial infarction sudden cardiac death in ST
elevation myocardial infarction with ventricular fibrillation | Marsman | 21461030 | European |
2011; Myocardial infarction women | Yamada | 21211798 | East Asian | 2010; Obesity | Neale B |
UKBB | European | 2017; Obesity | Foster MC | 22044751 | European | 2011; Obesity | Paternoster
L | 21935397 | European | 2011; Obesity | Cotsapas C | 19553259 | European | 2009; Obesity |
Wang K | 21552555 | European | 2011; Obesity | Melka MG | 22013104 | European | 2011; Obesity
| EGGC | 22484627 | European | 2012; Obesity | Meyre D | 19151714 | European | 2009; Obesity |
Jiao H | 21708048 | European | 2011; Obesity | Liu YZ | 19714249 | European | 2009; Obesity |
Dorajoo R | 21544081 | Mixed | 2011; Obesity | Scherag A | 20421936 | European | 2010; Obesity |
Hinney A | 18159244 | European | 2007; Obesity | Wan ES | 21037115 | European | 2010; Obesity |
Wen W | 22344219 | East Asian | 2012; Obesity | Okada Y | 22344221 | East Asian | 2012; Obesity |
GIANT | 23563607 | European | 2013; Obesity | Kim HJ | 23818313 | Mixed | 2012; Obesity and
osteoporosis | Liu YZ | 19714249 | European | 2009; Obesity body mass index | Jiao H | 21708048 |
European | 2011; Obesity body mass index | Paternoster L | 21935397 | European | 2011; Obesity
body mass index | Willer CJ | 19079261 | European | 2008; Obesity body mass index | Thorleifsson
G | 19079260 | Mixed | 2008; Obesity body mass index | Meyre D | 19151714 | European | 2009;
Obesity body mass index | Wang K | 21552555 | European | 2011; Obesity body mass index |
Scherag A | 20421936 | European | 2010; Obesity body mass index | Yanagiya | 17855449 | East
Asian | 2007; Obesity body mass index | Tanabe | 17200173 | East Asian | 2007; Obesity body mass
index 30 kgm2 | Speliotes EK | 20935630 | European | 2010; Obesity body mass index females |
Jiao H | 21708048 | European | 2011; Obesity body mass index males | Jiao H | 21708048 |
European | 2011; Obesity class 1 | GIANT | 23563607 | European | 2013; Obesity class 2 | GIANT |
23563607 | European | 2013; Obesity class 3 | GIANT | 23563607 | European | 2013; Obesity early
onset extreme | Wheeler E | 23563609 | European | 2013; Obesity early onset extreme | Scherag A |
20421936 | European | 2010; Obesity early onset extreme | Hinney A | 18159244 | European | 2007;
Obesity extreme | Paternoster L | 21935397 | European | 2011; Obesity extreme | Cotsapas C |
19553259 | European | 2009; Obesity in adult survivors of childhood cancer exposed to cranial
radiation | Wilson CL | 25963547 | Unspecified | 2015; Obesity in adult survivors of childhood
cancer not exposed to cranial radiation | Wilson CL | 25963547 | Unspecified | 2015; Obesity
related traits | Melka MG | 22013104 | European | 2011; Perioperative myocardial infarction in
coronary artery bypass surgery | Kertai MD | 25948407 | Unspecified | 2015; Response to
angiotensin II receptor blocker therapy | Turner ST | 22566498 | Mixed | 2012; Response to
angiotensin II receptor blocker therapy opposite direction w diuretic therapy | Turner ST |
22566498 | Mixed | 2012; Response to statins LDL cholesterol change | Postmus I | 25350695 |
European | 2014; Self-reported diabetes | Neale B | UKBB | European | 2017; Self-reported
hypertension | Neale B | UKBB | European | 2017; Smoking cessation | Argos M | 24665060 | South
Asian | 2014; Smoking cessation | Uhl GR | 20811658 | European | 2010; Smoking cessation in
chronic obstructive pulmonary disease | Siedlinski M | 21685187 | European | 2011; Statin response
difference HDL | Barber M | 20339536 | European | 2010; Statin response difference LDL | Barber
M | 20339536 | European | 2010; Statin response difference triglycerides | Barber M | 20339536 |
European | 2010; Systolic blood pressure response to hydrochlorothiazide in hypertension | Salvi E |
27802415 | Mixed | 2016; Triglyceride levels | Spracklen CN | 28334899 | East Asian | 2017;
Triglyceride levels | Kurano M | 26763881 | Asain | 2016; Triglyceride levels | Zhu Y | 28371326 |
East Asian | 2017; Triglyceride levels | Southam L | 28548082 | Mixed | 2017; Triglycerides | Prins
B | 28887542 | European | 2017; Triglycerides | GLGC | 24097068 | European | 2013; Triglycerides
| GLGC | 20686565 | Mixed | 2010; Triglycerides | Spracklen CN | 28334899 | East Asian | 2017;
Triglycerides | Kathiresan S | 19060906 | European | 2008; Triglycerides | Surakka I | 25961943 |
European | 2015; Triglycerides | Aulchenko YS | 19060911 | European | 2008; Triglycerides |
Waterworth DM | 20864672 | Mixed | 2010; Triglycerides | Willer CJ | 18193043 | European | 2008;
Triglycerides | Lu X | 26582766 | East Asian | 2015; Triglycerides | Kamatani Y | 20139978 | East
Asian | 2010; Triglycerides | Kathiresan S | 18193044 | Mixed | 2008; Triglycerides | Saxena R |
17463246 | European | 2007; Triglycerides | Coram MA | 23726366 | Mixed | 2013; Triglycerides |
Below JE | 26780889 | Hispanic | 2016; Triglycerides | Kim YJ | 21909109 | East Asian | 2011;
Triglycerides | Ko A | 24886709 | Hispanic | 2014; Triglycerides | van Leeuwen EM | 27036123 |
Mixed | 2016; Triglycerides | Kooner JS | 18193046 | Mixed | 2008; Triglycerides | Tan A |
22171074 | East Asian | 2011; Triglycerides | Pollin TI | 19074352 | European | 2008; Triglycerides
Blood Pressure TG BP | Kraja AT | 21386085 | European | 2011; Type 2 diabetes young onset and
obesity | Ma RC | 24465431 | East Asian | 2014; Urinary albumin to creatinine ratio in diabetes |
CKDGen | 26631737 | European | 2016; Waist Circumference Triglycerides WC TG | Kraja AT |
21386085 | European | 2011

Table 3: Full MR results for FIGS. 3, 4, 6, 9, 11, and 13

TABLE 3A
MR results for SLE risk on CAD (FIG. 3). Listed by: Exposure GWAS
| Outcome | MR Method | # SNPs used as IVs | Beta | SE | Pval.

SLE GWAS | Coronary artery disease || id:ebi-a-GCST005194 (CAD-a) Maximum
likelihood | 54 | 0.015290802 | 0.006143039 | 0.012805732; SLE GWAS | Coronary artery
disease || id:ebi-a-GCST005194 (CAD-a) | MR
Egger | 54 | 0.028049502 | 0.012616451 | 0.030569527; SLE GWAS | Coronary artery
disease || id:ebi-a-GCST005194 (CAD-a) | MR Egger
(bootstrap) | 54 | 0.020108073 | 0.009937195 | 0.025; SLE GWAS | Coronary artery
disease || id:ebi-a-GCST005194 (CAD-a) | Simple
median | 54 | 0.021556333 | 0.012183182 | 0.076835225; SLE GWAS | Coronary artery
disease || id:ebi-a-GCST005194 (CAD-a) | Weighted
median | 54 | 0.027935725 | 0.009420111 | 0.003021511; SLE GWAS | Coronary artery
disease || id:ebi-a-GCST005194 (CAD-a) | Penalised weighted
median | 54 | 0.029352045 | 0.009614673 | 0.002266879; SLE GWAS | Coronary artery
disease || id:ebi-a-GCST005194 (CAD-a) | Inverse variance
weighted | 54 | 0.014705928 | 0.006925477 | 0.03371587; SLE GWAS | Coronary artery
disease || id:ebi-a-GCST005194 (CAD-a) | Inverse variance weighted (multiplicative
random effects) | 54 | 0.014705928 | 0.006925477 | 0.03371587; SLE GWAS | Coronary
artery disease || id:ebi-a-GCST005194 (CAD-a) | Inverse variance weighted (fixed
effects) | 54 | 0.014705928 | 0.005902712 | 0.012724621; SLE GWAS | Coronary artery
disease || id:ebi-a-GCST005194 (CAD-a) | Simple
mode | 54 | 0.033278815 | 0.013822956 | 0.019576044; SLE GWAS | Coronary artery
disease || id:ebi-a-GCST005194 (CAD-a) | Weighted
mode | 54 | 0.033278815 | 0.009524991 | 0.000969901; SLE GWAS | Coronary artery
disease || id:ebi-a-GCST005194 (CAD-a) | Weighted mode
(NOME) | 54 | 0.033278815 | 0.009682061 | 0.001151373; SLE GWAS | Coronary artery
disease || id:ebi-a-GCST005194 (CAD-a) | Simple mode
(NOME) |54 | 0.033278815 | 0.014579917 | 0.026499411; SLE GWAS | Coronary artery
disease || id:ebi-a-GCST005194 (CAD-a) | Robust adjusted profile score
(RAPS) | 54 | 0.018834077 | 0.007533841 | 0.012421774; SLE GWAS | Coronary artery
disease || id:ebi-a-GCST005194 (CAD-a) | MR-PRESSO
(raw) | 54 | 0.0147 | 0.00693 | 0.0384; SLE GWAS | Coronary artery disease || id:ebi-a-
GCST005194 (CAD-a) | MR-PRESSO (outlier-corrected) | 53 | 0.0178 | 0.00635 | 0.00713;
SLE GWAS | Coronary artery disease || id:ebi-a-GCST005195 (CAD-b) | Maximum
likelihood | 55 | 0.00966599 | 0.007523088 | 0.198847049; SLE GWAS | Coronary artery
disease || id:ebi-a-GCST005195 (CAD-b) | MR
Egger | 55 | 0.035029704 | 0.014850198 | 0.022049416; SLE GWAS | Coronary artery
disease || id:ebi-a-GCST005195 (CAD-b) | MR Egger
(bootstrap) | 55 | 0.019640205 | 0.011803281 | 0.052; SLE GWAS | Coronary artery
disease || id:ebi-a-GCST005195 (CAD-b) | Simple
median | 55 | 0.006588173 | 0.015221679 | 0.665149066; SLE GWAS | Coronary artery
disease || id:ebi-a-GCST005195 (CAD-b) | Weighted
median | 55 | 0.038171371 | 0.011340109 | 0.00076253; SLE GWAS | Coronary artery
disease || id:ebi-a-GCST005195 (CAD-b) | Penalised weighted
median| 55 | 0.040183064 | 0.011041478 | 0.000273398; SLE GWAS | Coronary artery
disease || id:ebi-a-GCST005195 (CAD-b) Inverse variance
weighted | 55 | 0.009300709 | 0.008479458 | 0.272706157; SLE GWAS | Coronary artery
disease || id:ebi-a-GCST005195 (CAD-b) | Inverse variance weighted (multiplicative
random effects) | 55 | 0.009300709 | 0.008479458 | 0.272706157; SLE GWAS | Coronary
artery disease || id:ebi-a-GCST005195 (CAD-b) | Inverse variance weighted (fixed
effects) | 55 | 0.009300709 | 0.007241302 | 0.199002994; SLE GWAS | Coronary artery
disease || id:ebi-a-GCST005195 (CAD-b) | Simple
mode | 55 | 0.029495771 | 0.018333035 | 0.113472648; SLE GWAS | Coronary artery
disease || id:ebi-a-GCST005195 (CAD-b) | Weighted
mode | 55 | 0.038097903 | 0.011987018 | 0.002452246; SLE GWAS | Coronary artery
disease || id:ebi-a-GCST005195 (CAD-b) | Weighted mode
(NOME) | 55 | 0.038097903 | 0.010686392 | 0.000770675; SLE GWAS | Coronary artery
disease || id:ebi-a-GCST005195 (CAD-b) | Simple mode
(NOME) | 55 | 0.029495771 | 0.017286492 | 0.093699743; SLE GWAS | Coronary artery
disease || id:ebi-a-GCST005195 (CAD-b) | Robust adjusted profile score
(RAPS) | 55 | 0.014956473 | 0.008832582 | 0.090392758; SLE GWAS | Coronary artery
disease || id:ebi-a-GCST005195 (CAD-b) | MR-PRESSO
(raw) | 55 | 0.0093 | 0.00848 | 0.2776; SLE GWAS | Coronary artery disease || id:ebi-a-
GCST005195 (CAD-b) | MR-PRESSO (outlier-corrected) | 54 | 0.0131 | 0.00779 | 0.0985;
SLE GWAS | Myocardial infarction || id:ieu-a-798 (MI-a) | Maximum
likelihood | 56 | 0.03827387 | 0.011448214 | 0.00082813; SLE GWAS | Myocardial
infarction || id:ieu-a-798 (MI-a) | MR
Egger | 56 | 0.028690496 | 0.022642416 | 0.210552527; SLE GWAS | Myocardial
infarction || id:ieu-a-798 (MI-a) | MR Egger
(bootstrap) | 56 | 0.034193154 | 0.017986375 | 0.013; SLE GWAS | Myocardial infarction ||
id:ieu-a-798 (MI-a) | Simple median | 56 | 0.043835302 | 0.020695715 | 0.034167793; SLE
GWAS | Myocardial infarction | id:ieu-a-798 (MI-a) | Weighted
median | 56 | 0.034663127 | 0.016942928 | 0.040768582; SLE GWAS | Myocardial
infarction || id:ieu-a-798 (MI-a) | Penalised weighted
median | 56 | 0.03453482 | 0.015585901 | 0.026707049; SLE GWAS | Myocardial
infarction || id:ieu-a-798 (MI-a) | Inverse variance
weighted | 56 | 0.036889277 | 0.012364858 | 0.002850683; SLE GWAS | Myocardial
infarction || id:ieu-a-798 (MI-a) | Inverse variance weighted (multiplicative random
effects) | 56 | 0.036889277 | 0.012364858 | 0.002850683; SLE GWAS | Myocardial
infarction || id:ieu-a-798 (MI-a) | Inverse variance weighted (fixed
effects) | 56 | 0.036889277 | 0.011044441 | 0.000837554; SLE GWAS | Myocardial
infarction || id:ieu-a-798 (MI-a) | Simple
mode | 56 | 0.055681844 | 0.023073443 | 0.019170243; SLE GWAS | Myocardial
infarction || id:ieu-a-798 (MI-a) | Weighted
mode | 56 | 0.026582567 | 0.019367358 | 0.175465292; SLE GWAS | Myocardial
infarction || id:ieu-a-798 (MI-a) | Weighted mode
(NOME) | 56 | 0.026582567 | 0.017207625 | 0.128126387; SLE GWAS | Myocardial
infarction || id:ieu-a-798 (MI-a) | Simple mode
(NOME) | 56 | 0.055681844 | 0.024658491 | 0.027926489; SLE GWAS | Myocardial
infarction || id:ieu-a-798 (MI-a) | Robust adjusted profile score
(RAPS) | 56 | 0.038293116 | 0.012622377 | 0.002415357; SLE GWAS | Myocardial
infarction || id:ieu-a-798 (MI-a) | MR-PRESSO (raw) | 56 | 0.0369 | 0.0124 | 0.00424; SLE
GWAS | Myocardial infarction || id:ieu-a-798 (MI-a) | MR-PRESSO (outlier-
corrected) | | | |; SLE GWAS | Myocardial infarction | id:ebi-a-GCST011365 (MI-
b) | Maximum likelihood | 56 | 0.023162893 | 0.008152856 | 0.004496142; SLE
GWAS | Myocardial infarction | id:ebi-a-GCST011365 (MI-b) | MR
Egger | 56 | 0.031882017 | 0.01565068 | 0.046553924; SLE GWAS | Myocardial infarction
|| id:ebi-a-GCST011365 (MI-b) | MR Egger
(bootstrap) | 56 |0.022904806 | 0.012684503 | 0.04; SLE GWAS | Myocardial infarction ||
id:ebi-a-GCST011365 (MI-b) | Simple
median | 56 | 0.036037004 | 0.014729338 | 0.014420518; SLE GWAS | Myocardial
infarction || id:ebi-a-GCST011365 (MI-b) | Weighted
median | 56 | 0.036950539 | 0.01192361 | 0.00194215; SLE GWAS | Myocardial infarction
|| id:ebi-a-GCST011365 (MI-b) | Penalised weighted
median | 56 | 0.037575354 | 0.011983071 | 0.001714425; SLE GWAS | Myocardial
infarction | id:ebi-a-GCST011365 (MI-b) | Inverse variance
weighted | 56 | 0.022254954 | 0.008695698 | 0.010488136; SLE GWAS | Myocardial
infarction | id:ebi-a-GCST011365 (MI-b) | Inverse variance weighted (multiplicative
random effects) | 56 | 0.022254954 | 0.008695698 | 0.010488136; SLE
GWAS | Myocardial infarction | id:ebi-a-GCST011365 (MI-b) | Inverse variance weighted
(fixed effects) | 56 | 0.022254954 | 0.007850516 | 0.004584872; SLE GWAS | Myocardial
infarction || id:ebi-a-GCST011365 (MI-b) | Simple
mode | 56 | 0.047336088 | 0.017546482 | 0.009250518; SLE GWAS | Myocardial
infarction || id:ebi-a-GCST011365 (MI-b) | Weighted
mode | 56 | 0.03565302 | 0.013516383 | 0.010830508; SLE GWAS | Myocardial infarction
|| id:ebi-a-GCST011365 (MI-b) | Weighted mode
(NOME) | 56 | 0.03565302 | 0.011616673 | 0.003330472; SLE GWAS | Myocardial
infarction || id:ebi-a-GCST011365 (MI-b) | Simple mode
(NOME) | 56 | 0.047336088 | 0.018896136 | 0.01523431; SLE GWAS | Myocardial
infarction || id:ebi-a-GCST011365 (MI-b) | Robust adjusted profile score
(RAPS) | 56 | 0.027029541 | 0.009032275 | 0.002766566; SLE GWAS | Myocardial
infarction || id:ebi-a-GCST011365 (MI-b) | MR-PRESSO
(raw) | 56 | 0.0223 | 0.0087 | 0.0133; SLE GWAS | Myocardial infarction | id:ebi-a-
GCST011365 (MI-b) | MR-PRESSO (outlier-corrected) | | | |; SLE GWAS | Ischemic
stroke | id:ieu-a-1108 (IS) | Maximum
likelihood | 43 | 0.053573525 | 0.019411429 | 0.005781977; SLE GWAS | Ischemic stroke
|| id:ieu-a-1108 (IS) | MR Egger | 43 | 0.008927524 | 0.038599667 | 0.818243782; SLE
GWAS | Ischemic stroke | id:ieu-a-1108 (IS) | MR Egger
(bootstrap) | 43 | 0.004928575 | 0.034940833 | 0.443; SLE GWAS | Ischemic stroke
id:ieu-a-1108 (IS) | Simple median | 43 | 0.035673098 | 0.034732031 | 0.304375695; SLE
GWAS | Ischemic stroke | id:ieu-a-1108 (IS) | Weighted
median | 43 | 0.02157389 | 0.02801011 | 0.441170593; SLE GWAS | Ischemic stroke ||
id:ieu-a-1108 (IS) | Penalised weighted
median | 43 | 0.02140939 | 0.028647361 | 0.454856858; SLE GWAS | Ischemic stroke ||
id:ieu-a-1108 (IS) | Inverse variance
weighted | 43 | 0.051734409 | 0.020155559 | 0.010265472; SLE GWAS | Ischemic stroke ||
id:ieu-a-1108 (IS) | Inverse variance weighted (multiplicative random
effects) | 43 | 0.051734409 | 0.020155559 | 0.010265472; SLE GWAS | Ischemic stroke ||
id:ieu-a-1108 (IS) | Inverse variance weighted (fixed
effects) | 43 | 0.051734409 | 0.018770987 | 0.005849804; SLE GWAS | Ischemic stroke ||
id:ieu-a-1108 (IS) | Simple mode | 43 | 0.013110656 | 0.04073537 | 0.749163218; SLE
GWAS | Ischemic stroke | id:ieu-a-1108 (IS) | Weighted
mode | 43 | 0.013110656 | 0.028633959 | 0.649405321; SLE GWAS | Ischemic stroke ||
id:ieu-a-1108 (IS) | Weighted mode
(NOME) | 43 | 0.013110656 | 0.03066335 | 0.671150245; SLE GWAS | Ischemic stroke ||
id:ieu-a-1108 (IS) | Simple mode (NOME) | 43 | 0.013110656 | 0.04286415 | 0.761218189;
SLE GWAS | Ischemic stroke | id:ieu-a-1108 (IS) | Robust adjusted profile score
(RAPS) | 43 | 0.05030586 | 0.021213661 | 0.01772133; SLE GWAS | Ischemic stroke ||
id:ieu-a-1108 (IS) | MR-PRESSO (raw) | 43 | 0.0517 | 0.0202 | 0.0139; SLE
GWAS | Ischemic stroke || id:ieu-a-1108 (IS) | MR-PRESSO (outlier-corrected) | | | |;
SLE_GWAS | Cardiomyopathy || id:finn-b-I9_CARDMYO (CM) | Maximum
likelihood | 55 | 0.010479327 | 0.028441476 | 0.712536021;
SLE_GWAS | Cardiomyopathy || id:finn-b-I9_CARDMYO (CM) | MR Egger | 55 | −0.061381478
| 0.058103051 | 0.295566119; SLE_GWAS | Cardiomyopathy || id:finn-b-
I9_CARDMYO (CM) | MR Egger (bootstrap) | 55 | −0.017570857 | 0.045175691 | 0.354;
SLE_GWAS | Cardiomyopathy || id:finn-b-I9_CARDMYO (CM) | Simple
median | 55 | 0.046119875 | 0.053199274 | 0.385982136; SLE_GWAS | Cardiomyopathy ||
id:finn-b-I9 CARDMYO (CM) | Weighted
median | 55 | 0.012663363 | 0.040849612 | 0.756561269; SLE_GWAS | Cardiomyopathy ||
id:finn-b-I9 CARDMYO (CM) | Penalised weighted
median | 55 | 0.012705124 | 0.042748499 | 0.766308999; SLE_GWAS | Cardiomyopathy ||
id:finn-b-I9 CARDMYO (CM) | Inverse variance
weighted | 55 | 0.009816647 | 0.032528581 | 0.762815754; SLE_GWAS | Cardiomyopathy ||
id:finn-b-I9 CARDMYO (CM) | Inverse variance weighted (multiplicative random
effects) | 55 | 0.009816647 | 0.032528581 | 0.762815754; SLE_GWAS | Cardiomyopathy ||
id:finn-b-I9 CARDMYO (CM) | Inverse variance weighted (fixed
effects) | 55 | 0.009816647 | 0.027421722 | 0.720352315; SLE_GWAS | Cardiomyopathy ||
id:finn-b-I9 CARDMYO (CM) | Simple
mode | 55 | 0.003104534 | 0.058574964 | 0.957926675; SLE_GWAS | Cardiomyopathy ||
id:finn-b-I9 CARDMYO (CM) | Weighted
mode | 55 | 0.003104534 | 0.039283248 | 0.937301336; SLE_GWAS | Cardiomyopathy ||
id:finn-b-I9_CARDMYO (CM) | Weighted mode
(NOME) 55 | 0.003104534 | 0.041220643 | 0.940242401; SLE_GWAS | Cardiomyopathy ||
id:finn-b-I9 CARDMYO (CM) | Simple mode
(NOME) | 55 | 0.003104534 | 0.059180001 | 0.958356415; SLE_GWAS | Cardiomyopathy ||
id:finn-b-I9 CARDMYO (CM) | Robust adjusted profile score
(RAPS) | 55 | 0.015746425 | 0.034854658 | 0.651431878; SLE_GWAS | Cardiomyopathy ||
id:finn-b-I9 CARDMYO (CM) | MR-PRESSO
(raw) | 55 | 0.009816647 | 0.032528581 | 0.763974961; SLE_GWAS | Cardiomyopathy ||
id:finn-b-I9 CARDMYO (CM) | MR-PRESSO (outlier-corrected) | | | |;
SLE GWAS | Atrial fibrillation || id:ebi-a-GCST006414 (Afib) | Maximum
likelihood 55 | 0.009960258 | 0.007304858 | 0.172721375; SLE_GWAS | Atrial
fibrillation | id:ebi-a-GCST006414 (Afib) | MR Egger | 55 | −0.006803663
| 0.015766784 | 0.667841529; SLE_GWAS | Atrial fibrillation || id:ebi-a-
GCST006414 (Afib) | MR Egger (bootstrap) | 55 | 0.003561018 | 0.012060918 | 0.376;
SLE GWAS | Atrial fibrillation || id:ebi-a-GCST006414 (Afib) | Simple
median | 55 | 0.018361103 | 0.014396019 | 0.20215738; SLE_GWAS | Atrial fibrillation ||
id:ebi-a-GCST006414 (Afib) | Weighted
median 55 | 0.018324172 | 0.010573216 | 0.083082392; SLE_GWAS | Atrial fibrillation ||
id:ebi-a-GCST006414 (Afib) | Penalised weighted
median | 55 | 0.02042147 | 0.011255263 | 0.069617247; SLE_GWAS | Atrial fibrillation ||
id:ebi-a-GCST006414 (Afib) | Inverse variance
weighted | 55 | 0.009583089 | 0.008688878 | 0.270064303; SLE_GWAS | Atrial fibrillation ||
id:ebi-a-GCST006414 (Afib) | Inverse variance weighted (multiplicative random
effects) | 55 | 0.009583089 | 0.008688878 | 0.270064303; SLE_GWAS | Atrial fibrillation ||
id:ebi-a-GCST006414 (Afib) | Inverse variance weighted (fixed
effects) | 55 | 0.009583089 | 0.006993965 | 0.170626335; SLE_GWAS | Atrial fibrillation ||
id:ebi-a-GCST006414 (Afib) | Simple
mode | 55 | 0.023916455 | 0.016830679 | 0.161064914; SLE_GWAS | Atrial fibrillation ||
id:ebi-a-GCST006414 (Afib) | Weighted
mode | 55 | 0.016114424 | 0.011113512 | 0.152846369; SLE_GWAS | Atrial fibrillation ||
id:ebi-a-GCST006414 (Afib) | Weighted mode
(NOME) | 55 | 0.016114424 | 0.010433117 | 0.128296994; SLE_GWAS | Atrial fibrillation ||
id:ebi-a-GCST006414 (Afib) | Simple mode
(NOME) | 55 | 0.023916455 | 0.016820933 | 0.160826788; SLE_GWAS |Atrial fibrillation ||
id:ebi-a-GCST006414 (Afib) | Robust adjusted profile score
(RAPS) | 55 | 0.012365489 | 0.009560748 | 0.195886559; SLE_GWAS | Atrial fibrillation ||
id:ebi-a-GCST006414 (Afib) | MR-PRESSO
(raw) | 55 | 0.009583089 | 0.008688878 | 0.274955395; SLE_GWAS | Atrial fibrillation ||
id:ebi-a-GCST006414 (Afib) | MR-PRESSO (outlier-
corrected) | 54 | 0.014679252 | 0.008307351 | 0.082982525;

TABLE 3B
MR results for CAD risk on SLE using the MR base platform (FIG. 4). Listed
by: Exposure GWAS | Outcome | Method | # SNPs | Beta | SE | Pval.

Coronary artery disease || id:ebi-a-GCST005194 (CAD-a) | SLE_GWAS | Maximum
likelihood | 111 | −0.114181591 | 0.061408 | 0.062972148; Coronary artery disease || id:ebi-
a-GCST005194 (CAD-a) | SLE_GWAS | MR Egger | 111 | −0.236410779
| 0.174998 | 0.179513016; Coronary artery disease || id:ebi-a-GCST005194
(CAD-a) |SLE_GWAS | MR Egger (bootstrap) | 111 −0.091224492 |0.128159 | 0.247;
Coronary artery disease || id:ebi-a-GCST005194 (CAD-a) | SLE_GWAS | Simple
median | 111 | −0.272875839 | 0.10721 | 0.010919999; Coronary artery disease || id:ebi-a-
GCST005194 (CAD-a) | SLE_GWAS | Weighted median | 111 | −0.272540023
| 0.113663 | 0.016494655; Coronary artery disease || id:ebi-a-GCST005194
(CAD-a) | SLE_GWAS | Penalised weighted median | 111 | −0.275705165
| 0.121999 | 0.023827113; Coronary artery disease || id:ebi-a-GCST005194
(CAD-a) | SLE_GWAS | Inverse variance weighted | 111 | −0.112473913
| 0.075922 | 0.138489157; Coronary artery disease || id:ebi-a-GCST005194
(CAD-a) |SLE_GWAS | Inverse variance weighted (multiplicative random effects) | 111 | −0.112473913
| 0.075922 | 0.138489157; Coronary artery disease || id:ebi-a-GCST005194
(CAD-a) | SLE_GWAS | Inverse variance weighted (fixed effects) | 111 | −0.112473913
| 0.060631 | 0.063587319; Coronary artery disease || id:ebi-a-GCST005194
(CAD-a) | SLE_GWAS | Simple mode | 111 | −0.483646162 | 0.23513 | 0.04205842;
Coronary artery disease || id:ebi-a-GCST005194 (CAD-a) | SLE_GWAS | Weighted
mode | 111 | −0.33653999 | 0.125393 | 0.008401889; Coronary artery disease || id:ebi-a-
GCST005194 (CAD-a) | SLE_GWAS | Weighted mode (NOME) | 111 | −0.33653999
| 0.124527 | 0.00797245; Coronary artery disease ||
(CAD-a) | Simple id:ebi-a-GCST005194
SLE_GWAS | mode (NOME) | 111 | −0.483646162
| 0.254464 | 0.059966164; Coronary artery disease || id:ebi-a-GCST005194
(CAD-a) | SLE_GWAS | Robust adjusted profile score (RAPS) | 111 | −0.180808859
| 0.075862 | 0.017154628; Coronary artery disease ||id:ebi-a-GCST005195
(CAD-b) | SLE_GWAS Maximum likelihood | 49 | −0.155218502
| 0.073449 | 0.034576442; Coronary artery disease || id:ebi-a-GCST005195
(CAD-b) | SLE GWAS | MR Egger | 49 | − 0.285002506 | 0.213854 | 0.189056416;
Coronary artery disease || id:ebi-a-GCST005195 (CAD-b) | SLE_GWAS | MR Egger
(bootstrap) | 49 | −0.289358792 | 0.145914 | 0.029; Coronary artery disease || id:ebi-a-
GCST005195 (CAD-b) | SLE_GWAS | Simple median | 49 | −0.204998406
| 0.121811 | 0.092391041; Coronary artery disease || id:ebi-a-GCST005195
(CAD-b) | SLE_GWAS | Weighted median | 49 | −0.271983691 | 0.121545 | 0.025239528;
Coronary artery disease || id:ebi-a-GCST005195 (CAD-b) SLE GWAS | Penalised
weighted median | 49 | −0.27911421 | 0.12441 | 0.02486451; Coronary artery disease ||
id:ebi-a-GCST005195 (CAD-b) | SLE_GWAS | Inverse variance weighted | 49 | −0.153829903
| 0.086335 | 0.074786318; Coronary artery disease || id:ebi-a-GCST005195
(CAD-b) | SLE_GWAS | Inverse variance weighted (multiplicative random effects) | 49 | −0.153829903
| 0.086335 | 0.074786318; Coronary artery disease || id:ebi-a-GCST005195
(CAD-b) | SLE_GWAS |Inverse variance weighted (fixed effects) | 49 | −0.153829903
| 0.072625 | 0.034162617; Coronary artery disease || id:ebi-a-GCST005195
(CAD-b) | SLE_GWAS | Simple mode | 49| −0.392781259 | 0.241161 | 0.109920942;
Coronary artery disease || id:ebi-a-GCST005195 (CAD-b) | SLE_GWAS | Weighted
mode | 49 | −0.303433337 | 0.133885 | 0.027974766; Coronary artery disease || id:ebi-a-
GCST005195 (CAD-b) | SLE_GWAS | Weighted mode (NOME) | 49 | −0.312368129
| 0.134241 | 0.024235802; Coronary artery disease || id:ebi-a-GCST005195
(CAD-b) | SLE_GWAS | Simple mode (NOME) | 49 | −0.392781259
| 0.254327 | 0.129061148; Coronary artery disease || id:ebi-a-GCST005195
(CAD-b) | SLE_GWAS | Robust adjusted profile score (RAPS) | 49 | −0.177130259
| 0.088581 | 0.045539897; Myocardial infarction || id:ebi-a-GCST011365 (MI-
a) | SLE_GWAS | Maximum likelihood | 57 | 0.004270697 | 0.066415 | 0.948728509;
Myocardial infarction || nid:ebi-a-GCST011365 (MI-a) | SLE_GWAS | MR Egger | 57 | −0.020200346
| 0.216482 | 0.925994972; Myocardial infarction || id:ebi-a-GCST011365 (MI-
a) | SLE_GWAS | MR Egger (bootstrap) | 57 | −0.134616328 | 0.132849 | 0.149; Myocardial
infarction || id:ebi-a-GCST011365 (MI-a) | SLE_GWAS | Simple
median | 57 | 0.092035537 | 0.113557 | 0.417664981; Myocardial infarction || id:ebi-a-
GCST011365 (MI-a) | SLE_GWAS | Weighted median | 57 | −0.225030554
| 0.106627 | 0.034820252; Myocardial infarction || id:ebi-a-GCST011365 (MI-
a) | SLE_GWAS | Penalised weighted median | 57 | −0.237111727 | 0.11494 | 0.039120006;
Myocardial infarction || id:ebi-a-GCST011365 (MI-a) | SLE_GWAS | Inverse variance
weighted | 57 | 0.004039881 | 0.090597 | 0.964432675; Myocardial infarction || id:ebi-a-
GCST011365 (MI-a) | SLE_GWAS | Inverse variance weighted (multiplicative random
effects) | 57 | 0.004039881 | 0.090597 | 0.964432675; Myocardial infarction || id:ebi-a-
GCST011365 (MI-a) | SLE_GWAS | Inverse variance weighted (fixed
effects) | 57 | 0.004039881 | 0.06528 | 0.950654403; Myocardial infarction || id:ebi-a-
GCST011365 (MI-a) | SLE_GWAS | Simple mode | 57 | −0.349376544
| 0.247592 | 0.163747956; Myocardial infarction || id:ebi-a-GCST011365 (MI-
a) | SLE_GWAS | Weighted mode | 57 | −0.258778805 | 0.120762 | 0.036482689;
Myocardial infarction || id:ebi-a-GCST011365 (MI-a) | SLE_GWAS | Weighted mode
(NOME) | 57 | −0.26884522 | 0.121513 | 0.031028007; Myocardial infarction || id:ebi-a-
GCST011365 (MI-a) | SLE_GWAS | Simple mode (NOME) | 57 | −0.349376544
| 0.260291 | 0.184930907; Myocardial infarction || id:ebi-a-GCST011365 (MI-
a) | SLE_GWAS | Robust adjusted profile score (RAPS) | 57 | −0.025673122
| 0.091116 | 0.77812603; Myocardial infarction || id:ieu-a-798 (MI-
b) | SLE_GWAS | Maximum likelihood | 17 | −0.02377718 | 0.08646 | 0.783310481;
Myocardial infarction || id:ieu-a-798 (MI-b) | SLE_GWAS | MR Egger | 17 | −0.588263575
| 0.328514 | 0.093543667; Myocardial infarction || id:ieu-a-798 (MI-
b) | SLE_GWAS | MR Egger (bootstrap) | 17 | −0.131464626 | 0.144343 | 0.17; Myocardial
infarction || id:ieu-a-798 (MI-b) | SLE_GWAS | Simple
median | 17 | 0.099199518 | 0.150685 | 0.510329252; Myocardial infarction || id:ieu-a-798
(MI-b) | SLE_GWAS | Weighted median | 17 | −0.223573654 | 0.118887 | 0.06003209;
Myocardial infarction || id:ieu-a-798 (MI-b) | SLE_GWAS | Penalised weighted
median | 17 | −0.236006061 | 0.12217 | 0.053386993; Myocardial infarction || id:ieu-a-798
(MI-b) | SLE GWAS | Inverse variance weighted | 17 | −0.022734651
| 0.139514 | 0.870552985; Myocardial infarction || id:ieu-a-798 (MI-
b) | SLE_GWAS | Inverse variance weighted (multiplicative random effects) | 17 | −0.022734651
| 0.139514 | 0.870552985; Myocardial infarction || id:ieu-a-798 (MI-
b) | SLE_GWAS | Inverse variance weighted (fixed effects) | 17 | −0.022734651
| 0.08423 | 0.78722782; Myocardial infarction || id:ieu-a-798 (MI-
b) | SLE_GWAS | Simple mode | 17 | −0.063824419 | 0.18504 | 0.73464535; Myocardial
infarction || id:ieu-a-798 (MI-b) | SLE_GWAS | Weighted mode | 17 | −0.217935206
| 0.124668 | 0.099599948; Myocardial infarction || id:ieu-a-798 (MI-
b) | SLE_GWAS | Weighted mode (NOME) | 17 | −0.217935206 | 0.13007 | 0.113260261;
Myocardial infarction || id:ieu-a-798 (MI-b) | SLE_GWAS | Simple mode (NOME) | 17 | −0.063824419
| 0.19626 | 0.749241169; Myocardial infarction || id:ieu-a-798 (MI-
b) | SLE_GWAS | Robust adjusted profile score(RAPS) | 17 | −0.098128658
| 0.128011 | 0.443341691; Ischemic stroke || id:ieu-a-
1108 | SLE_GWAS | Robust adjusted profile score (RAPS) | 1 | −0.150879014
| 0.266619 | 0.571462911;

TABLE 3C
SSMR results for positive causal SNPs (FIG. 6). Listed by: Exposure
GWAS | Outcome | SNP ID |chr |position | Beta | SE | Pval.

SLE GWAS | Coronary artery disease || id:ebi-a-
GCST005194 | rs7692514 | 4 | 66142596 | 2.417829648 | 1.174090117 | 0.039463399; SLE
GWAS | Coronary artery disease || id:ebi-a-
GCST005194 | rs11037296 | 11 | 43183773 | 2.040133167 | 0.994941791 | 0.040315171;
SLE GWAS | Coronary artery disease || id:ebi-a-
GCST005194 | rs75010564 | 5 | 38543824 | 2.032226217 | 0.975074615 | 0.037144244;
SLE GWAS | Coronary artery disease || id:ebi-a-
GCST005194 | rs78506915 | 13 | 44588317 | 1.646246203 | 0.691827392 | 0.017333225;
SLE GWAS | Coronary artery disease || id:ebi-a-
GCST005194 | rs73161005 | 22 | 30143240 | 1.567787065 | 0.703494196 | 0.025842434;
SLE GWAS | Coronary artery disease || id:ebi-a-
GCST005194 | rs706778 | 10 | 6098949 | 1.517537479 | 0.572845274 | 0.0080701; SLE
GWAS | Coronary artery disease || id:ebi-a-
GCST005194 | rs7810922 | 7 | 101597464 | 1.502437353 | 0.706444054 | 0.033439983;
SLE GWAS | Coronary artery disease || id:ebi-a-
GCST005194 | rs965355 | 15 | 60059824 | 1.333288777 | 0.63679464 | 0.036282256; SLE
GWAS |Coronary artery disease || id:ebi-a-
GCST005194 | rs12743484 | 1 | 26335929 | 1.158260605 | 0.346488215 | 0.000829206;
SLE GWAS | Coronary artery disease || id:ebi-a-
GCST005194 | rs34099611 | 8 | 144886628 | 0.955317508 | 0.311839394 | 0.0021877; SLE
GWAS | Coronary artery disease || id:ebi-a-
GCST005194 | rs11603249 | 11 | 43173812 | 0.910050407 | 0.348457962 | 0.009010632;
SLE GWAS | Coronary artery disease || id:ebi-a-
GCST005194 | rs11037278 | 11 | 43173294 | 0.882985711 | 0.345074875 | 0.010502704;
SLE GWAS | Coronary artery disease || id:ebi-a-
GCST005194 | rs11601828 | 11 | 43167522 | 0.698610448 | 0.262616336 | 0.007809561;
SLE GWAS | Coronary artery disease || id:ebi-a-
GCST005194 | rs12279248 | 11 | 43178098 | 0.670564043 | 0.260066663 | 0.009925001;
SLE GWAS | Coronary artery disease || id:ebi-a-
GCST005194 | rs10905718 | 10 | 6114856 | 0.616079867 | 0.308039934 | 0.045500264;
SLE GWAS | Coronary artery disease || id:ebi-a-
rs10954650 | 7 | 139326552 | 0.561592444 | 0.199602134 | 0.004899634;
GCST005194 | Coronary artery disease || id:ebi-a-
SLE GWAS | rs1609265 | 2 | 229140950 | 0.554840437 | 0.210117089 | 0.008275326;
GCST005194 | rs10954650 | 7 | 139326552 | 0.561592444 | 0.199602134 | 0.004899634;
SLE GWAS | Coronary artery disease || id:ebi-a-
GCST005194 | rs13026988 | 2 | 242407179 | 0.524378487 | 0.246965352 | 0.033729753;
SLE GWAS | Coronary artery disease || id:ebi-a-
GCST005194 | rs10933559 | 2 | 242407746 | 0.510846139 | 0.246965352 | 0.038593674;
SLE GWAS | Coronary artery disease || id:ebi-a-
GCST005194 | rs73245892 | 4 | 46916798 | 0.502989724 | 0.177411104 | 0.004580205;
SLE GWAS | Coronary artery disease || id:ebi-a-
GCST005194 rs1981601 | 7 | 22202485 | 0.46659019 | 0.145331371 | 0.001324921; SLE
GWAS | Coronary artery disease || id:ebi-a-
GCST005194 | rs2541117 | 12 | 11831208 | 0.405970442 | 0.19283596 | 0.035268407; SLE
GWAS | Coronary artery disease || id:ebi-a-GCST005194
| rs6946131 | 7 | 54783373 | 0.395821181 | 0.19283596 | 0.040108322; SLE
GWAS | Coronary artery disease || id:ebi-a-
GCST005194 | rs4869313 | 5 | 96224121 | 0.341520043 | 0.097822324 | 0.000480806; SLE
GWAS |Coronary artery disease || id:ebi-a-
GCST005194 | rs2122775 | 11 | 103399264 | 0.337775574 | 0.155150503 | 0.02947435;
SLE GWAS | Coronary artery disease || id:ebi-a-
GCST005194 | rs2594113 | 3 | 71663248 | 0.313587795 | 0.116826826 | 0.007270131; SLE
GWAS | Coronary artery disease || id:ebi-a-
GCST005194 | rs7179733 | 15 | 32373693 | 0.298311761 | 0.150430717 | 0.04736175; SLE
GWAS | Coronary artery disease || id:ebi-a-
GCST005194 | rs4459332 | 11 | 72508267 | 0.282853459 | 0.109239957 | 0.009617526;
SLE GWAS | Coronary artery disease || id:ebi-a-
GCST005194 | rs209665 | 20 | 32128889 | 0.27294016 | 0.107226492 | 0.010913557; SLE
GWAS | Coronary artery disease || id:ebi-a-
GCST005194 | rs874610 | 9 | 100782695 | 0.270081478 | 0.090945804 | 0.002980936; SLE
GWAS | Coronary artery disease || id:ebi-a-
GCST005194 | rs6478522 | 9 | 100790547 | 0.265947578 | 0.092323771 | 0.003969228;
Coronary artery disease || id:ebi-a-
rs11164848 | 1 | 93421110 | 0.263192298 | 0.109176064 | 0.015921315;
SLE GWAS | Coronary artery disease || id:ebi-a-GCST005194 SLE GWAS |
GCST005194 | rs11145763 | 9 | 139263596 | 0.260200322 | 0.093736762 | 0.005505556;
SLE GWAS | Coronary artery disease || id:ebi-a-
GCST005194 | rs116967545 | 10 | 64503831 | 0.255815775 | 0.10979958 | 0.019814454;
SLE GWAS | Coronary artery disease || id:ebi-a-
GCST005194 | rs10800816 | 1 | 202125363 | 0.254149585 | 0.127074793 | 0.045500264;
SLE GWAS | Coronary artery disease || id:ebi-a-
GCST005194 | rs12470957 | 2 | 85773252 | 0.241774622 | 0.105004306 |0.021305808;
SLE GWAS | Coronary artery disease || id:ebi-a-
GCST005194 | rs6689858 | 1 | 197376021 | 0.240265357 | 0.102970867 | 0.019630657;
SLE GWAS | Coronary artery disease || id:ebi-a-
GCST005194 | rs1237290 | 1 | 114135147 | 0.2290673 | 0.071958314 | 0.001455899; SLE
GWAS | Coronary artery disease || id: ebi-a-
GCST005194 | rs9851386 | 3 | 174764910 | 0.227877301 | 0.106665971 | 0.032649786;
SLE GWAS | Coronary artery disease || id:ebi-a-
GCST005194 | rs1292043 | 17 | 57934169 | 0.22047468 | 0.078906728 | 0.005204154; SLE
GWAS | Coronary artery disease || id:ebi-a-
GCST005194 | rs144484752 | 14 | 37439749 | 0.218966846 | 0.11055679 | 0.04763811;
SLE GWAS | Coronary artery disease || id:ebi-a-
GCST005194 | rs16837131 | 2 | 202173812 | 0.217516258 | 0.094863919 | 0.021852076;
SLE GWAS | Coronary artery disease || id:ebi-a-
GCST005194 | rs13023380 | 2 | 163154363 | 0.217348974 | 0.055998207 | 0.000103876;
SLE GWAS | Coronary artery disease || id:ebi-a-
GCST005194 | rs2019097 | 2 | 202193463 | 0.214641594 | 0.091031033 | 0.018378904;
SLE GWAS | Coronary artery disease || id:ebi-a-
GCST005194 | rs62131887 | 19 | 10587596 | 0.201183142 | 0.08818987 | 0.022533658;
SLE GWAS | Coronary artery disease || id:ebi-a-
GCST005194 | rs6738825 | 2 | 198896895 | 0.196686058 | 0.068360398 |0.004012301;
SLE GWAS | Coronary artery disease || id:ebi-a-
GCST005194 | rs425648 | 19 | 18202112 | 0.193879069 | 0.051701085 | 0.000176835; SLE
GWAS | Coronary artery disease || id:ebi-a-
GCST005194 | rs2022013 | 1 | 183353853 | 0.193088143 | 0.068360398 | 0.004734538;
SLE GWAS | Coronary artery disease || id:ebi-a-
GCST005194 | rs2456973 | 12 | 56416928 | 0.190304461 | 0.069623583 | 0.006269685;
SLE GWAS | Coronary artery disease || id:ebi-a-
GCST005194 | rs223881 | 16 57386566 | 0.182589941 | 0.043120872 | 2.29E−05; SLE
GWAS | Coronary artery disease || id:ebi-a-
GCST005194 | rs56114296 | 17 | 26110748 | 0.160270766 | 0.066397889 | 0.015787424;
SLE GWAS | Coronary artery disease || id:ebi-a-
GCST005194 | rs1534154 | 3 | 119311030 | 0.139900324 | 0.068991941 | 0.042582933;
SLE GWAS | Coronary artery disease || id:ebi-a-
GCST005194 | rs10916668 | 1 | 20233086 | 0.13218404 | 0.061429927 | 0.031414236; SLE
GWAS | Coronary artery disease || id:ebi-a-
GCST005194 | rs3807134 | 7 | 128577338 | 0.117331184 | 0.03279443 | 0.000346528; SLE
GWAS | Coronary artery disease || id:ebi-a-
GCST005194 | rs13107612 | 4 | 102739980 | 0.095782481 | 0.034986415 | 0.006186956;
SLE GWAS | Coronary artery disease || id:ebi-a-
GCST005194 | rs13106926 | 4 | 102739791 | 0.095208933 | 0.034986415 | 0.006502346;
SLE GWAS | Coronary artery disease || id:ebi-a-
GCST005194 | rs34749007 | 4 | 102754507 | 0.094635385 | 0.035559963 | .007784181;
SLE GWAS | Coronary artery disease || id:ebi-a-
GCST005194 | rs35388091 | 4 | 102739452 | 0.091194099 | 0.034986415 | 0.00914575;
SLE GWAS | Coronary artery disease || id:ebi-a-
GCST005194 | rs4637409 | 4 | 102753408 | 0.090047003 | 0.035559963 | 0.011333055;
SLE GWAS | Coronary artery disease || id:ebi-a-
GCST005194 | rs34029191 | 4 | 102752201 | 0.087752812 | 0.035559963 | 0.01359683;
SLE GWAS | Coronary artery disease || id:ebi-a-
GCST005194 | rs17266594 | 4 | 102750922 | 0.085458621 | 0.035559963 | 0.016251149;
SLE GWAS | Coronary artery disease || id:ebi-a-
GCST005194 | rs1125271 | 4 | 102745985 | 0.084311525 | 0.033265772 | 0.011261347;
SLE GWAS | Coronary artery disease || id:ebi-a-
GCST005194 | rs17200824 | 4 | 102752589 | 0.083737977 | 0.035559963 | 0.018530747;
SLE GWAS | Coronary artery disease || id:ebi-a-
GCST005194 | rs13129744 | 4 | 102756731 | 0.079723143 | 0.036133511 | 0.027359559;
SLE GWAS | Coronary artery disease || id:ebi-a-
GCST005194 | rs10516487 | 4 | 102751076 | 0.0780025 | 0.032118676 | 0.015158439; SLE
GWAS | Coronary artery disease || id:ebi-a-
GCST005194 | rs12119966 | 1 | 183378051 | 0.07688472 | 0.02987937
GCST005194 | rs12119966 | 1 | 183378051
GWAS | Coronary artery disease || id:ebi-a-
GCST005194 | rs34725611 | 19 | 10477067 | 0.07423989 | 0.02672636 | 0.005473204; SLE
GWAS | Coronary artery disease || id:ebi-a-
GCST005194 | rs11085727 | 19 | 10466123 | 0.070421838 | 0.02672636 | 0.008415696;
SLE GWAS | Coronary artery disease || id:ebi-a-
GCST005194 | rs2078087 | 1 | 183358405 | 0.069251875 | 0.031373778 | 0.027291915;
SLE GWAS | Coronary artery disease || id:ebi-a-
GCST005194 | rs12409487 | 1 | 183345350 | 0.066682008 | 0.02987937 | 0.025634313;
SLE GWAS | Coronary artery disease || id:ebi-a-
GCST005194 | rs1122259 | 1 | 183356931 | 0.065224478 | 0.02987937 | 0.029041199; SLE
GWAS | Coronary artery disease || id:ebi-a-
GCST005194 | rs4411998 | 4 | 102738147 | 0.062516709 | 0.029824485 | 0.036068546;
SLE GWAS | Coronary artery disease || id:ebi-a-
GCST005194 | rs11150610 | 16 | 31334236 | 0.055523646 | 0.027524543 | 0.04367033;
SLE GWAS | Coronary artery disease || id:ebi-a-
GCST005194 | rs34815476 | 7 | 128563333 | 0.052471088 | 0.023320484 | 0.024448945;
SLE GWAS | Coronary artery disease || id:ebi-a-
GCST005194 | rs11150608 | 16 | 31301125 | 0.051088189 | 0.025992237 | 0.049354402;
SLE GWAS | Coronary artery disease || id:ebi-a-
GCST005194 | rs729302 | 7 | 128568960 | 0.04846288 | 0.022227336 | 0.029233167; SLE
GWAS | Coronary artery disease || id: ebi-a-
GCST005194 | rs2061831 | 8 | 11339882 | 0.047402643 | 0.024071655 | 0.048926597; SLE
GWAS | Coronary artery disease || id: ebi-a-
GCST005194 | rs13426947 | 2 | 191933254 | 0.037877497 | 0.016200616 | 0.019385787;
SLE GWAS | Coronary artery disease || id:ebi-a-
GCST005194 | rs10168266 | 2 | 191935804 | 0.033253848 | 0.015740155 | 0.034628504;
SLE GWAS | Coronary artery disease || id:ebi-a-
GCST005194 | rs10931481 | 2 | 191954852 | 0.032990895 | 0.016775031 | 0.049221652;

TABLE 3D
SSMR results for negative causal SNPs (FIG. 6). Listed by: Exposure
GWAS | Outcome | SNP ID | chr | position |Beta | SE | Pval.

SLE GWAS | Coronary artery disease || id:ebi-a-GCST005194 rs6756736 | 2 | 43558743 | −1.67158593
| 0.606944891 | 0.005885408; SLE GWAS | Coronary artery disease || id:ebi-a-
GCST005194 | rs12946196 | 17 | 75807910 | −1.572086767 | 0.676594305 | 0.02015073;
SLE GWAS | Coronary artery disease || id:ebi-a-
GCST005194 | rs8105429 | 19 | 18481333 | −1.527587396 | 0.683394361 | 0.025398046;
SLE GWAS | Coronary artery disease || id:ebi-a-
GCST005194 | rs34709472 | 19 | 55704944 | −1.212581614 |0.507079584 | 0.016788629;
SLE GWAS | Coronary artery disease || id:ebi-a-
GCST005194 | rs4678000 | 3 | 121890735 | −1.164140201 | 0.567145226 | 0.040108322;
SLE GWAS | Coronary artery disease || id:ebi-a-
GCST005194 | rs7566072 | 2 | 236203142 | −0.955317508 | 0.346488215 | 0.005830887;
SLE GWAS | Coronary artery disease || id:ebi-a-
GCST005194 | rs10878246 | 12 | 40632099 | −0.752425412 | 0.363588119 | 0.0385044;
SLE GWAS | Coronary artery disease || id:ebi-a-
GCST005194 | rs4917385 | 10 | 105003721 | −0.732226072 | 0.302990099 | 0.015663354;
SLE GWAS | Coronary artery disease || id:ebi-a-
GCST005194 | rs4748857 | 10 | 23599558 | −0.678126935 | 0.326688889 | 0.037916378;
SLE GWAS | Coronary artery disease || id:ebi-a-GCST005194 | rs928596 | 1 | 1155947875
| −0.656478547 | 0.323189439 | 0.042229641; SLE GWAS | Coronary artery disease || id:ebi-
a-GCST005194 | rs64491734 | 9966105 | −0.636020359 | 0.230049917 | 0.005697414;
SLE GWAS | Coronary artery disease | id:ebi-a-GCST005194 | rs7442295 | 4 | 9966380 | −0.625871098
| 0.230049917 | 0.006516588; SLE GWAS | Coronary artery disease || id:ebi-
a-GCST005194 | rs73066671 | 19 | 55696684 | −0.625871098 | 0.311244005 | 0.044339236;
SLE GWAS | Coronary artery disease || id:ebi-a-GCST005194 | rs7821169 | 8 | 59187800 | −0.622213596
| 0.27926122 | 0.025875836; SLE GWAS | Coronary artery disease || id:ebi-a-
GCST005194 | rs1052690 | 9 | 86258685 | −0.610778843 | 0.153719507 | 7.09E−05;
SLE GWAS | Coronary artery disease || id:ebi-a-GCST005194 | rs3785437 | 17 | 73906222 | −0.598806401
| 0.304477831 | 0.049221652; SLE GWAS | Coronary artery disease || id:ebi-
a-GCST005194 | rs7540556 | 1 | 169531442 | −0.574538914 | 0.187135532 | 0.002139331;
SLE GWAS | Coronary artery disease || id:ebi-a-
GCST005194 | rs11057864 | 12 | 125335950 | −0.518499074 | 0.13996294 | 0.00021177;
SLE GWAS | Coronary artery disease || id:ebi-a-
GCST005194 | rs7915387 | 10 | 73010024 | −0.502311165 | 0.190418612 | 0.008341354;
SLE GWAS | Coronary artery disease || id:ebi-a-
GCST005194 | rs1053093 | 1 | 183905073 | −0.466866008 | 0.189452873 | 0.013728663;
SLE GWAS | Coronary artery disease || id:ebi-a-
GCST005194 | rs144705368 | 2 | 10642477 | −0.430160843 | 0.162314025 | 0.008044969;
SLE GWAS | Coronary artery disease || id:ebi-a-
GCST005194 | rs7927370 | 11 | 55136219 | −0.428788378 | 0.159821123 | 0.007298096;
SLE GWAS | Coronary artery disease || id:ebi-a-
GCST005194 | rs3766374 | 1 | 160720554 | −0.426268964 | 0.213134482 | 0.045500264;
SLE GWAS | Coronary artery disease || id:ebi-a-
GCST005194 | rs7121755 | 11 | 115455636 | −0.377554144 | 0.190418612 | 0.047394398;
SLE GWAS | Coronary artery disease || id:ebi-a-
GCST005194 | rs7899961 | 10 | 60026425 | −0.363633991 | 0.176160467 | 0.038996845;
SLE GWAS | Coronary artery disease || id:ebi-a-GCST005194 | rs6945400 | 7 | 51079850
| −0.355224136 | 0.179303612 | 0.047576464; SLE GWAS | Coronary artery disease || id:ebi-
a-GCST005194 | rs3733345 | 4 | 954247 | −0.347023918 | 0.113075209 | 0.002148014; SLE
GWAS | Coronary artery disease || id:ebi-a-GCST005194 | rs4293757 | 4 | 30943209 | −0.333153736
| 0.161677549 | 0.039340638; SLE GWAS | Coronary artery disease || id:ebi-
a-GCST005194 | rs55816332 | 7 | 31423490 | −0.331073713 | 0.165536857 | 0.045500264;
SLE GWAS | Coronary artery disease || id:ebi-a-GCST005194 | rs500600 | 11 | 30481057
| −0.320905437 | 0.13718095 |0.019320652; SLE GWAS | Coronary artery disease | id:ebi-a-
GCST005194 | rs79821347 | 3 | 50531835 | −0.295176991 | 0.114843781 | 0.010162575;
SLE GWAS | Coronary artery disease || id:ebi-a-GCST005194 | rs12683801 | 9 | 5272381 | −0.288992674
| 0.122975606 | 0.018773411; SLE GWAS | Coronary artery disease || id:ebi-
a-GCST005194 | rs7214635 | 17 | 4601020 | −0.264563369 | 0.10050452 | 0.008479572;
SLE GWAS | Coronary artery disease || id:ebi-a-
GCST005194 | rs9415635 | 10 | 63753482 | −0.25734358 | 0.115024782 | 0.025267514;
SLE GWAS | Coronary artery disease || id:ebi-a-
GCST005194 | rs55634455 | 2 | 136593694 | −0.256301811 | 0.126772939 | 0.043203309;
SLE GWAS | Coronary artery disease || id:ebi-a-
GCST005194 | rs10419198 | 19 | 50038017 | −0.252167911 | 0.089567837 | 0.004871889;
SLE GWAS | Coronary artery disease || id:ebi-a-
GCST005194 | rs138191786 | 7 | 27415597 | −0.244485902 | 0.109043008 | 0.02495457;
SLE GWAS | Coronary artery disease || id:ebi-a-
GCST005194 | rs956835313 | 50481713 | −0.206479056 | 0.09529802 | 0.03026028;
SLE GWAS |Coronary artery disease || id:ebi-a-
GCST005194 | rs9952980 | 18 | 42888797 | −0.205941734 | 0.101254686 | 0.04196185;
SLE GWAS | Coronary artery disease || id:ebi-a-
GCST005194 | rs79558495 | 7 | 100365435 | −0.199358297 | 0.089806773 | 0.026428398;
SLE GWAS | Coronary artery disease || id:ebi-a-GCST005194 | rs13723721 | 30120947 | −0.192915342
| 0.090945804 | 0.033903956; SLE GWAS | Coronary artery disease || id:ebi-
a-GCST005194 | rs16953685 | 15 | 70034088 | −0.17161888 | 0.067498639 | 0.011004585;
SLE GWAS | Coronary artery disease || id:ebi-a-
GCST005194 | rs72832915 | 17 | 37834808 | −0.170215992 | 0.084458317 | 0.043864365;
SLE GWAS | Coronary artery disease || id:ebi-a-GCST005194 | rs2235947 | 20 | 8628696 | −0.169652049
0.06786082 | 0.012419331; SLE GWAS | Coronary artery disease || id:ebi-a-
GCST005194 | rs35281701 | 15 | 69992054 | −0.168746597 | 0.06893478 | 0.014368488;
SLE GWAS | Coronary artery disease || id:ebi-a-
GCST005194 | rs4948496 | 10 | 63805617 | −0.162419036 | 0.040068132 | 5.04E−05; SLE
GWAS | Coronary artery disease || id:ebi-a-GCST005194 | rs7625006 | 3 | 51372940 | −0.161797154
| 0.061818724 | 0.008863258; SLE GWAS | Coronary artery disease || id:ebi-
a-GCST005194 | rs7264711 | 20 | 8630944 | −0.161169446 | 0.06786082 | 0.01754895; SLE
GWAS | Coronary artery disease || id:ebi-a-GCST005194 | rs55705316 | 1 | 206933517 | −0.160594822
| 0.074120687 | 0.03026028; SLE GWAS | Coronary artery disease || id:ebi-a-
GCST005194 | rs6574349 | 14 | 77497666 | −0.151626519 | 0.058317892 | 0.009322376;
SLE GWAS | Coronary artery disease || id:ebi-a-
GCST005194 | rs118087357 | 20 | 54465778 | −0.148090469 | 0.063182311 | 0.019085352;
SLE GWAS | Coronary artery disease || id:ebi-a-
GCST005194 | rs2941509 | 17 | 37921194 | −0.143730251 | 0.04277686 | 0.000779425;
SLE GWAS | Coronary artery disease || id:ebi-a-GCST005194 | rs6705304 | 2 | 43596746 | −0.142447922
| 0.072082081 | 0.048133208; SLE GWAS | Coronary artery disease || id:ebi-
a-GCST005194 | rs28392589 | 17 | 37992885 | −0.136086262 | 0.040693756 | 0.000825334;
SLE GWAS | Coronary artery disease || id:ebi-a
GCST005194 | rs12946188 | 17 | 37984430 | −0.135822017 | 0.040693756 | 0.000844864;
SLE GWAS | Coronary artery disease || id:ebi-a-
GCST005194 | rs113115305 | 17 | 37970687 | −0.135822017 | 0.042543472 | 0.001410242;
SLE GWAS | Coronary artery disease || id:ebi-a-
GCST005194 | rs113370572 | 17 | 37933468 | −0.135557772 | 0.042543472 | 0.001440874;
SLE GWAS | Coronary artery disease || id:ebi-a-
GCST005194 | rs12946792 | 17 | 37985322 | −0.135293527 | 0.040693756 | 0.000885216;
SLE GWAS | Coronary artery disease || id:ebi-a-
GCST005194 | rs12978179 | 19 | 2183175 | −0.135109217 | 0.05461862 | 0.013372784;
SLE GWAS | Coronary artery disease || id:ebi-a-
GCST005194 | rs112345383 | 17 | 37942018 | −0.135029281 | 0.042543472 | 0.001503987;
SLE GWAS | Coronary artery disease || id:ebi-a-
GCST005194 | rs600751 | 11 | 126059460 | −0.134724842 | 0.059210281 | 0.022884204;
SLE GWAS | Coronary artery disease || id:ebi-a-
GCST005194 | rs28621669 | 17 | 38011266 | −0.134236546 | 0.040693756 | 0.000971332;
SLE GWAS | Coronary artery disease || id:ebi-a-
GCST005194 | rs28723017 | 17 | 38011451 | −0.134236546 | 0.040693756 | 0.000971332;
SLE GWAS | Coronary artery disease || id:ebi-a-
GCST005194 | rs12947480 | 17 | 38009676 | −0.133972301 | 0.040693756 | 0.000994041;
SLE GWAS | Coronary artery disease || id:ebi-a-
GCST005194 | rs12938117 | 17 | 38008165 | −0.133708056 | 0.040693756 | 0.001017241;
SLE GWAS | Coronary artery disease || id:ebi-a-
GCST005194 | rs8079075 | 17 | 38010815 | −0.13344381 | 0.040693756 | 0.001040942;
SLE GWAS | Coronary artery disease || id:ebi-a-
GCST005194 | rs111469562 | 17 | 37982697 | −0.132893895 | 0.041067019 | 0.001212068;
SLE GWAS | Coronary artery disease || id:ebi-a-
GCST005194 | rs113233720 | 17 | 38008191 | −0.130088074 | 0.041067019 | 0.00153649;
SLE GWAS | Coronary artery disease || id:ebi-a-
GCST005194 | rs112880843 | 17 | 38024298 | −0.127027178 | 0.041067019 | 0.001980322;
SLE GWAS | Coronary artery disease || id:ebi-a-
GCST005194 | rs4252665 | 17 | 37885383 | −0.120465656 | 0.046571774 | 0.009690929;
SLE GWAS | Coronary artery disease || id:ebi-a-
GCST005194 | rs143123127 | 17 | 38009000 | −0.1085098 | 0.034255055 | 0.00153649;
SLE GWAS | Coronary artery disease || id:ebi-a-
GCST005194 | rs9907966 | 17 | 38018803 | −0.10226288 | 0.036994324 | 0.005704756;
SLE GWAS | Coronary artery disease || id:ebi-a-
GCST005194 | rs1453560 | 17 | 38023441 | −0.096618733 | 0.035794684 | 0.006949634;
SLE GWAS | Coronary artery disease | id:ebi-a-GCST005194 | rs1131665 | 11 | 613208 | −0.089217483
| 0.03084647 | 0.003824232; SLE GWAS | Coronary artery disease || id:ebi-a-
GCST005194 | rs35865896 | 11 | 584591 | −0.088183004 | 0.03325759 | 0.008013152; SLE
GWAS | Coronary artery disease || id:ebi-a-GCST005194 | rs12272314 | 11 | 590648 | −0.085895555
| 0.03084647 | 0.005359125; SLE GWAS | Coronary artery disease | id:ebi-a-
GCST005194 | rs1055382 | 11 | 612182 | −0.085420994 | 0.03084647 | 0.005618882; SLE
GWAS | Coronary artery disease || id:ebi-a-GCST005194 | rs1061502 | 11 | 614318 | −0.084946433
| 0.03084647 | 0.005889945; SLE GWAS | Coronary artery disease || id:ebi-a-
GCST005194 | rs34889521 | 11 | 617519 | −0.084946433 | 0.03084647 | 0.005889945; SLE
GWAS | Coronary artery disease || id:ebi-a-GCST005194 | rs11246213 | 11 | 612967 | −0.084471872
| 0.03084647 | 0.006172738; SLE GWAS | Coronary artery disease || id:ebi-a-
GCST005194 | rs12805435 | 11 | 612355 | −0.083997311 | 0.03084647 | 0.0064677; SLE
GWAS | Coronary artery disease | id:ebi-a-GCST005194 | rs11246217 | 11 | 623765 | −0.077080426
| 0.029577373 | 0.009159025; SLE GWAS | Coronary artery disease || id:ebi-
a-GCST005194 | rs58688157 | 11 | 625085 | −0.073943432 | 0.029577373 | 0.012419331;
SLE GWAS | Coronary artery disease || id:ebi-a-GCST005194 | rs1124816 | 11 | 619671 | −0.067669444
| 0.030921799 | 0.028640056; SLE GWAS | Coronary artery disease | id:ebi-
a-GCST005194 | 11 | 609353 | −0.06677316 | 0.030921799 | 0.030817575;
SLE GWAS | Coronary artery disease || id:ebi-a-GCST005194 | rs36060383 | 11 | 598723 | −0.06453245
| 0.030921799 | 0.036892065; SLE GWAS | Coronary artery disease || id:ebi-a-
GCST005194 | rs2396545 | 11 | 601785 | −0.064084308 | 0.030921799 | 0.038222212; SLE
GWAS | Coronary artery disease || id:ebi-a-GCST005194 | rs936469 | 11 | 606749 | −0.064084308
| 0.030921799 | 0.038222212; SLE GWAS | Coronary artery disease || id:ebi-
a-GCST005194 | rs4963128 | 11 | 589564 | −0.063663805 | 0.032118676 | 0.047463254;
SLE GWAS | Coronary artery disease || id:ebi-a-GCST005194 | rs12795572 | 11 | 590999 | −0.063188024
| 0.030921799 0.041005117; SLE GWAS | Coronary artery disease || id:ebi-
a-GCST005194 | rs12419694 | 11 | 598033 | −0.058258462 | 0.029577373 | 0.04887311;
SLE GWAS | Coronary artery disease || id:ebi-a-
GCST005194 | rs10229001 | 7 | 128599397 | −0.053876948 | 0.024605221 | 0.028549254;
SLE GWAS | Coronary artery disease || id:ebi-a-
GCST005194 | rs10954213 | 7 | 128589427 | −0.052604265 | 0.024605221 | 0.032522341;
SLE GWAS | Coronary artery disease || id:ebi-a-
GCST005194 | rs10239340 | 7 | 128668510 | −0.051755809 | 0.024180993 | 0.032326421;
SLE GWAS | Coronary artery disease || id:ebi-a-
GCST005194 | rs4731536 | 7 | 128640293 | −0.050058897 | 0.024180993 0.038435918;
SLE GWAS | Coronary artery disease || id:ebi-a-
GCST005194 | rs4731533 | 7 | 128602142 | −0.049102203 | 0.02334367 | 0.035426602;

TABLE 3E
46 PPI-based cluser MR analysis (FIG. 9). Listed by: Cluster
| Exposure_data | number of SNPs linking to each
cluster | Outcome | MR method | # SNPs used as IVs | beta | SE | pval.

1 | SLE GWAS | 130 | Coronary artery disease ∥ id: ebi-a-GCST005194 | Maximum
likelihood | 112 | −0.040596674 | 0.004210508 | 5.32706E−22; 1 | SLE
GWAS | 130 | Coronary artery disease ∥ id: ebi-a-GCST005194 | MR Egger | 112
| −0.049736772 | 0.011322688 | 2.58806E−05; 1 | SLE GWAS | 130 | Coronary artery disease
∥ id: ebi-a-GCST005194 | MR Egger (bootstrap) | 112 | −0.025229339 | 0.008356851 | 0;
1 | SLE GWAS | 130 | Coronary artery disease ∥ id: ebi-a-GCST005194 | Simple
median | 112 | −0.052020716 | 0.008025622 | 9.06168E−11; 1 | SLE
GWAS | 130 | Coronary artery disease ∥ id: ebi-a-GCST005194 | Weighted median | 112
| −0.051050324 | 0.0067614 | 4.3439E−14; 1 | SLE GWAS | 130 | Coronary artery disease ∥
id: ebi-a-GCST005194 | Penalised weighted median | 112 | −0.05446263
| 0.006478375 | 4.21224E−17; 1 | SLE GWAS | 130 | Coronary artery disease ∥
id: ebi-a-GCST005194 | Inverse variance weighted | 112 | −0.039402541
| 0.006130859 | 1.30215E−10; 1 | SLE GWAS | 130 | Coronary artery disease
∥ id: ebi-a-GCST005194 | Inverse variance weighted (multiplicative random
effects) | 112 | −0.039402541 | 0.006130859 | 1.30215E−10; 1 | SLE
GWAS | 130 | Coronary artery disease ∥ id: ebi-a-GCST005194 | Inverse variance weighted
(fixed effects) | 112 | −0.039402541 | 0.003869825 | 2.3862E−24; 1 | SLE
GWAS | 130 | Coronary artery disease ∥ id: ebi-a-GCST005194 | Simple mode | 112
| −0.058996198 | 0.015915729 | 0.000329209; 1 | SLE GWAS | 130 | Coronary artery disease
∥ id: ebi-a-GCST005194 | Weighted mode | 112 | −0.053095706
| 0.01507099 | 0.000620578; 1 | SLE GWAS | 130 | Coronary artery disease ∥
id: ebi-a-GCST005194 | Weighted mode (NOME) | 112 | −0.058996198 |
0.01316617 | 1.8155E−05; 1 | SLE GWAS | 130 | Coronary artery disease ∥
id: ebi-a-GCST005194 | Simple mode (NOME) | 112 | −0.058996198 |
0.011812704 | 2.21267E−06; 1 | SLE GWAS | 130 | Coronary artery disease
∥ id: ebi-a-GCST005194 | Robust adjusted profile score (RAPS) | 112
| −0.050416442 | 0.006516204 | 1.01719E−14; 1 | SLE GWAS | 130 | Coronary artery disease
∥ id: ebi-a-GCST005194 | Raw | 112 | −0.039402541 | 0.006130859 | 3.36211E−09; 1 | SLE
GWAS | 130 | Coronary artery disease ∥ id: ebi-a-GCST005194 | Outlier-corrected | 108
| −0.049649417 | 0.005833342 | 1.16018E−13; 2 | SLE GWAS | 111 | Coronary artery disease
∥ id: ebi-a-GCST005194 | Maximum
likelihood | 100 | 0.014137052 | 0.00317366 | 8.4091E−06; 2 | SLE GWAS | 111 | Coronary
artery disease ∥ id: ebi-a-GCST005194 | MR
Egger | 100 | 0.013926524 | 0.006991596 | 0.049165205; 2 | SLE GWAS | 111 | Coronary
artery disease ∥ id: ebi-a-GCST005194 | MR Egger
(bootstrap) | 100 | 0.019237226 | 0.006363077 | 0.001; 2 | SLE GWAS | 111 | Coronary
artery disease ∥ id: ebi-a-GCST005194 | Simple
median | 100 | 0.020162768 | 0.005419767 | 0.000199043; 2 | SLE GWAS | 111 | Coronary
artery disease ∥ id: ebi-a-GCST005194 | Weighted
median | 100 | 0.014302374 | 0.004359956 | 0.001036621; 2 | SLE GWAS | 111 | Coronary
artery disease ∥ id: ebi-a-GCST005194 | Penalised weighted
median | 100 | 0.014221578 | 0.004475635 | 0.001485225; 2 | SLE GWAS | 111 | Coronary
artery disease ∥ id: ebi-a-GCST005194 | Inverse variance
weighted | 100 | 0.013764509 | 0.004006626 | 0.0005916; 2 | SLE GWAS | 111 | Coronary
artery disease ∥ id: ebi-a-GCST005194 | Inverse variance weighted (multiplicative random
effects) | 100 | 0.013764509 | 0.004006626 | 0.0005916; 2 | SLE GWAS | 111 | Coronary
artery disease ∥ id: ebi-a-GCST005194 | Inverse variance weighted (fixed
effects) | 100 | 0.013764509 | 0.003105941 | 9.35053E−06; 2 | SLE GWAS | 111 | Coronary
artery disease ∥ id: ebi-a-GCST005194 | Simple
mode | 100 | 0.024476764 | 0.007356246 | 0.001231574; 2 | SLE GWAS | 111 | Coronary
artery disease ∥ id: ebi-a-GCST005194 | Weighted
mode | 100 | 0.01324961 | 0.006672417 | 0.049829055; 2 | SLE GWAS | 111 | Coronary
artery disease ∥ id: ebi-a-GCST005194 | Weighted mode
(NOME) | 0.01324961 | 0.00489074 | 0.007950569; 2 | SLE GWAS | 111 | Coronary
artery disease ∥ id: ebi-a-GCST005194 | Simple mode
(NOME) | 100 | 0.024476764 | 0.006240812 | 0.000162012; 2 | SLE
GWAS | 111 | Coronary artery disease ∥ id: ebi-a-GCST005194 | Robust adjusted profile
score (RAPS) | 100 | 0.013240963 | 0.004047694 | 0.001070785; 2 | SLE
GWAS | 111 | Coronary artery disease ∥ id: ebi-a-
GCST005194 | Raw | 100 | 0.013764509 | 0.004006626 | 0.000865874; 2 | SLE
GWAS | 111 | Coronary artery disease ∥ id: ebi-a-GCST005194 | Outlier-
corrected | 98 | | 0.013019763 | 0.003760107 | 0.000797519; 3 | SLE
GWAS | 175 | Coronary artery disease ∥ id: ebi-a-GCST005194 | Maximum
likelihood | 147 | 0.020700948 | 0.00228433 | 1.28E−19; 3 | SLE GWAS | 175 | Coronary
artery disease ∥ id: ebi-a-GCST005194 | MR
Egger | 147 | 0.011641466 | 0.004845728 | 0.017552338; 3 | SLE GWAS | 175 | Coronary
artery disease ∥ id: ebi-a-GCST005194 | MR Egger
(bootstrap) | 147 | 0.020427756 | 0.005248426 | 0; 3 | SLE GWAS | 175 | Coronary artery
disease ∥ id: ebi-a-GCST005194 | Simple
median | 147 | 0.025224122 | 0.003692108 | 8.37958E−12; 3 | SLE GWAS | 175 | Coronary
artery disease ∥ id: ebi-a-GCST005194 | Weighted
median | 147 | 0.021605791 | 0.003200074 | 1.46169E−11; 3 | SLE GWAS | 175 | Coronary
artery disease ∥ id: ebi-a-GCST005194 | Penalised weighted
median | 147 | 0.021568007 | 0.003284696 | 5.16117E−11; 3 | SLE GWAS | 175 | Coronary
artery disease ∥ id: ebi-a-GCST005194 | Inverse variance
weighted | 147 | 0.020519045 | 0.002797373 | 2.22E−13; 3 | SLE GWAS | 175 | Coronary
artery disease ∥ id: ebi-a-GCST005194 | Inverse variance weighted (multiplicative random
effects) | 147 | 0.020519045 | 0.002797373 | 2.22E−13; 3 | SLE GWAS | 175 | Coronary
artery disease ∥ id: ebi-a-GCST005194 | Inverse variance weighted (fixed
effects) | 147 | 0.020519045 | 0.0022404 | 5.25E−20; 3 | SLE GWAS | 175 | Coronary artery
disease ∥ id: ebi-a-GCST005194 | Simple
mode | 147 | 0.024113792 | 0.006023053 | 9.89414E−05; 3 | SLE GWAS | 175 | Coronary
artery disease ∥ id: ebi-a-GCST005194 | Weighted
mode | 147 | 0.024113792 | 0.005275778 | 1.02775E−05; 3 | SLE GWAS | 175 | Coronary
artery disease ∥ id: ebi-a-GCST005194 | Weighted mode
(NOME) | 147 | 0.024113792 | 0.004681372 | 8.2564E−07; 3 | SLE GWAS | 175 | Coronary
artery disease ∥ id: ebi-a-GCST005194 | Simple mode
(NOME) | 147 | 0.024113792 | 0.005479776 | 2.07184E−05; 3 | SLE
GWAS | 175 | Coronary artery disease ∥ id: ebi-a-GCST005194 | Robust adjusted profile
score (RAPS) | 147 | 0.019734864 | 0.002470426 | 1.37E−15; 3 | SLE
GWAS | 175 | Coronary artery disease ∥ id: ebi-a-
GCST005194 | Raw | 147 | 0.020519045 | 0.002797373 | 1.4124E−11; 3 | SLE
GWAS | 175 | Coronary artery disease ∥ id: ebi-a-GCST005194 | Outlier-
corrected | 143 | 0.019130599 | 0.002051171 | 1.97853E−16; 4 | SLE GWAS | 30 | Coronary
artery disease ∥ id: ebi-a-GCST005194 | Maximum
likelihood | 28 | 0.027340095 | 0.005110471 | 8.80424E−08; 4 | SLE GWAS | 30 | Coronary
artery disease ∥ id: ebi-a-GCST005194 | MR
Egger | 28 | 0.020203984 | 0.015519441 | 0.204384289; 4 | SLE GWAS | 30 | Coronary
artery disease ∥ id: ebi-a-GCST005194 | MR Egger
(bootstrap) | 28 | 0.033000482 | 0.018482083 | 0.037; 4 | SLE GWAS | 30 | Coronary artery
disease ∥ id: ebi-a-GCST005194 | Simple
median | 28 | 0.027575883 | 0.006755909 | 4.46993E−05; 4 | SLE GWAS | 30 | Coronary
artery disease ∥ id: ebi-a-GCST005194 | Weighted
median | 28 | 0.027999724 | 0.006669798 | 2.69299E−05; 4 | SLE GWAS | 30 | Coronary
artery disease ∥ id: ebi-a-GCST005194 | Penalised weighted
median | 28 | 0.027999724 | 0.006631993 | 2.42233E−05; 4 | SLE GWAS | 30 | Coronary
artery disease ∥ id: ebi-a-GCST005194 | Inverse variance
weighted | 28 | 0.027210874 | 0.005066274 | 7.83084E−08; 4 | SLE GWAS | 30 | Coronary
artery disease ∥ id: ebi-a-GCST005194 | Inverse variance weighted (multiplicative random
effects) | 28 | 0.027210874 | 0.002479042 | 4.96499E−28; 4 | SLE GWAS | 30 | Coronary
artery disease ∥ id: ebi-a-GCST005194 | Inverse variance weighted (fixed
effects) | 28 | 0.027210874 | 0.005066274 | 7.83084E−08; 4 | SLE GWAS | 30 | Coronary
artery disease ∥ id: ebi-a-GCST005194 | Simple
mode | 28 | 0.028366579 | 0.01044271 | 0.011373055; 4 | SLE GWAS | 30 | Coronary
artery disease ∥ id: ebi-a-GCST005194 | Weighted
mode | 28 | 0.029015657 | 0.010652253 | 0.011173384; 4 | SLE GWAS | 30 | Coronary
artery disease ∥ id: ebi-a-GCST005194 | Weighted mode
(NOME) | 28 | 0.029015657 | 0.01056911 | 0.01062077; 4 | SLE GWAS | 30 | Coronary
artery disease ∥ id: ebi-a-GCST005194 | Simple mode
(NOME) | 28 | 0.028366579 | 0.011403071 | 0.019331393; 4 | SLE GWAS | 30 | Coronary
artery disease ∥ id: ebi-a-GCST005194 | Robust adjusted profile score
(RAPS) | 28 | 0.027308332 | 0.005271592 | 2.21551E−07; 4 | SLE GWAS | 30 | Coronary
artery disease ∥ id: ebi-a-GCST005194 | Raw | 28 | 0.027210874 | 0.002479042 | 1.85498E−11;
4 | SLE GWAS | 30 | Coronary artery disease ∥ id: ebi-a-GCST005194 | Outlier-
corrected | NA | NA | NA | NA; 5 | SLE GWAS | 383 | Coronary artery disease ∥ id: ebi-a-
GCST005194 | Maximum likelihood | 332 | 0.007169151 | 0.002155343 | 0.000880318;
5 | SLE GWAS | 383 | Coronary artery disease ∥ id: ebi-a-GCST005194 | MR Egger | 332
| −0.024067023 | 0.007895042 | 0.002486778; 5 | SLE GWAS | 383 | Coronary artery disease
∥ id: ebi-a-GCST005194 | MR Egger (bootstrap) | 332 | −0.009474606 |
0.006434155 | 0.079; 5 | SLE GWAS | 383 | Coronary artery disease ∥
id: ebi-a-GCST005194 | Simple median | 332 | 0.017446507 | 0.003890442 | 7.3101E−06;
5 | SLE GWAS | 383 | Coronary artery disease ∥ id: ebi-a-GCST005194 | Weighted
median | 332 | 0.004087708 | 0.003482348 | 0.240460399; 5 | SLE GWAS | 383 | Coronary
artery disease ∥ id: ebi-a-GCST005194 | Penalised weighted
median | 332 | 0.00585235 | 0.003522655 | 0.096643886; 5 | SLE GWAS | 383 | Coronary
artery disease ∥ id: ebi-a-GCST005194 | Inverse variance
weighted | 332 | 0.007093381 | 0.003290484 | 0.031105106; 5 | SLE
GWAS | 383 | Coronary artery disease ∥ id: ebi-a-GCST005194 | Inverse variance weighted
(multiplicative random effects) | 332 | 0.007093381 | 0.003290484 | 0.031105106; 5 | SLE
GWAS | 383 | Coronary artery disease ∥ id: ebi-a-GCST005194 | Inverse variance weighted
(fixed effects) | 332 0.007093381 | 0.002045789 | 0.000525697; 5 | SLE
GWAS | 383 | Coronary artery disease ∥ id: ebi-a-GCST005194 | Simple
mode | 332 | 0.038702154 | 0.013159703 | 0.003502412; 5 | SLE GWAS | 383 | Coronary
artery disease ∥ id: ebi-a-GCST005194 | Weighted
mode | 332 | 0.006063549 | 0.012145722 | 0.617946658; 5 | SLE GWAS | 383 | Coronary
artery disease ∥ id: ebi-a-GCST005194 | Weighted mode
(NOME) | 332 | 0.016943084 | 0.010246695 | 0.099173752; 5 | SLE
GWAS | 383 | Coronary artery disease ∥ id: ebi-a-GCST005194 | Simple mode
(NOME) | 332 | 0.038702154 | 0.012292129 | 0.00179031; 5 | SLE GWAS | 383 | Coronary
artery disease ∥ id: ebi-a-GCST005194 | Robust adjusted profile score
(RAPS) | 332 | 0.006960397 | 0.003381289 | 0.039541794; 5 | SLE GWAS | 383 | Coronary
artery disease ∥ id: ebi-a-
GCST005194 | Raw | 332 0.007093381 | 0.003290484 | 0.031825456; 5 | SLE
GWAS | 383 | Coronary artery disease ∥ id: ebi-a-GCST005194 | Outlier-
corrected | 322 | 0.004448755 | 0.002819951 | 0.115642392; 6 | SLE GWAS | 5 | Coronary
artery disease ∥ id: ebi-a-GCST005194 | Maximum
likelihood | 4 | 0.086382094 | 0.043626564 | 0.047699672; 6 | SLE GWAS | 5 | Coronary
artery disease ∥ id: ebi-a-GCST005194 | MR
Egger | 4 | 0.073283809 | 0.082603898 | 0.468585591; 6 | SLE GWAS | 5 | Coronary artery
disease ∥ id: ebi-a-GCST005194 | MR Egger
(bootstrap) | 4 | 0.076401959 | 0.131670962 | 0.236; 6 | SLE GWAS | 5 | Coronary artery
disease ∥ id: ebi-a-GCST005194 | Simple
median | 4 | 0.083826967 | 0.121587723 | 0.490548864; 6 | SLE GWAS | 5 | Coronary
artery disease ∥ id: ebi-a-GCST005194 | Weighted
median | 4 | 0.083605432 | 0.0782942 | 0.28559411; 6 | SLE GWAS | 5 | Coronary artery
disease ∥ id: ebi-a-GCST005194 | Penalised weighted
median | 4 | 0.083605432 | 0.061838159 | 0.176374065; 6 | SLE GWAS | 5 | Coronary
artery disease ∥ id: ebi-a-GCST005194 | Inverse variance
weighted | 4 | 0.081469907 | 0.039192013 | 0.037641485; 6 | SLE GWAS | 5 | Coronary
artery disease ∥ id: ebi-a-GCST005194 | Inverse variance weighted (multiplicative random
effects) | 4 | 0.081469907 | 0.032515264 | 0.012224742; 6 | SLE GWAS | 5 | Coronary
artery disease ∥ id: ebi-a-GCST005194 | Inverse variance weighted (fixed
effects) | 4 | 0.081469907 | 0.039192013 | 0.037641485; 6 | SLE GWAS | 5 | Coronary
artery disease ∥ id: ebi-a-GCST005194 | Simple
mode | 4 | 0.07036745 | 0.062527309 | 0.342331723; 6 | SLE GWAS | 5 | Coronary artery
disease ∥ id: ebi-a-GCST005194 | Weighted
mode | 4 | 0.082227423 | 0.045510464 | 0.168532328; 6 | SLE GWAS | 5 | Coronary artery
disease ∥ id: ebi-a-GCST005194 | Weighted mode
(NOME) | 4 | 0.082227423 | 0.041372043 | 0.141024215; 6 | SLE GWAS | 5 | Coronary
artery disease ∥ id: ebi-a-GCST005194 | Simple mode
(NOME) | 4 | 0.07036745 | 0.059530375 | 0.322347383; 6 | SLE GWAS | 5 | Coronary
artery disease ∥ id: ebi-a-GCST005194 | Robust adjusted profile score
(RAPS) | 4 | 0.086375697 | 0.046853293 | 6.53E−02; 6 | SLE GWAS | 5 | Coronary artery
disease ∥ id: ebi-a-GCST005194 | Raw | 4 | 0.081469907 | 0.032515264 | 0.087275745;
6 | SLE GWAS | 5 | Coronary artery disease ∥ id: ebi-a-GCST005194 | Outlier-
corrected | NA | NA | NA | NA; 7 | SLE GWAS | 39 | Coronary artery disease ∥ id: ebi-a-
GCST005194 | Maximum likelihood | 33 | 0.034857511 | 0.013371841 | 0.00913968;
7 | SLE GWAS | 39 | Coronary artery disease ∥ id: ebi-a-GCST005194 | MR
Egger | 33 | 0.028461602 | 0.041865706 | 0.501657031; 7 | SLE GWAS | 39 | Coronary
artery disease ∥ id: ebi-a-GCST005194 | MR Egger
(bootstrap) | 33 | 0.047688951 | 0.028144988 | 0.047; 7 | SLE GWAS | 39 | Coronary artery
disease ∥ id: ebi-a-GCST005194 | Simple
median | 33 | 0.0134151 | 0.02122223 | 0.527305281; 7 | SLE GWAS | 39 | Coronary artery
disease ∥ id: ebi-a-GCST005194 | Weighted
median | 33 | 0.016796287 | 0.018634586 | 0.367402168; 7 | SLE GWAS | 39 | Coronary
artery disease ∥ id: ebi-a-GCST005194 | Penalised weighted
median | 33 | 0.014975311 | 0.018467854 | 0.417431534; 7 | SLE GWAS | 39 | Coronary
artery disease ∥ id: ebi-a-GCST005194 | Inverse variance
weighted | 33 | 0.024458475 | 0.018815389 | 0.193628857; 7 | SLE GWAS | 39 | Coronary
artery disease ∥ id: ebi-a-GCST005194 | Inverse variance weighted (multiplicative random
effects) | 33 | 0.024458475 | 0.018815389 | 0.193628857; 7 | SLE GWAS | 39 | Coronary
artery disease ∥ id: ebi-a-GCST005194 | Inverse variance weighted (fixed
effects) | 33 | 0.024458475 | 0.011004658 | 0.026245715; 7 | SLE GWAS | 39 | Coronary
artery disease ∥ id: ebi-a-GCST005194 | Simple
mode | 33 | 0.009121752 | 0.025855856 | 0.726557317; 7 | SLE GWAS | 39 | Coronary
artery disease ∥ id: ebi-a-GCST005194 | Weighted
mode 33 | 0.009121752 | 0.018155739 | 0.618814328; 7 | SLE GWAS | 39 | Coronary
artery disease ∥ id: ebi-a-GCST005194 | Weighted mode
(NOME) | 33 | 0.009121752 | 0.02140677 | 0.67287656; 7 | SLE GWAS | 39 | Coronary
artery disease ∥ id: ebi-a-GCST005194 | Simple mode
(NOME) | 33 | 0.009121752 | 0.029951078 | 0.762676991; 7 | SLE GWAS | 39 | Coronary
artery disease ∥ id: ebi-a-GCST005194 | Robust adjusted profile score
(RAPS) | 33 | 0.011053874 | 0.021681714 | 0.610174282; 7 | SLE GWAS | 39 | Coronary
artery disease ∥ id: ebi-a-
GCST005194 | Raw | 33 | 0.024458475 | 0.018815389 | 0.202914943; 7 | SLE
GWAS | 39 | Coronary artery disease ∥ id: ebi-a-GCST005194 | Outlier-corrected | 30
| −0.001417548 | 0.015347896 | 0.927046267; 8 | SLE GWAS | 67 | Coronary artery disease ∥
id: ebi-a-GCST005194 | Maximum
likelihood | 57 | 0.019716672 | 0.007800171 | 0.011480483; 8 | SLE GWAS | 67 | Coronary
artery disease ∥ id: ebi-a-GCST005194 | MR Egger | 57 | −0.018950473 |
0.022745198 | 0.40835603; 8 | SLE GWAS | 67 | Coronary artery disease ∥
id: ebi-a-GCST005194 | MR Egger (bootstrap) | 57 | 0.012954469 | 0.025989027 | 0.31;
8 | SLE GWAS | 67 | Coronary artery disease ∥ id: ebi-a-GCST005194 | Simple
median | 57 | 0.0134151 | 0.013638453 | 0.325300771; 8 | SLE GWAS | 67 | Coronary
artery disease ∥ id: ebi-a-GCST005194 | Weighted
median | 57 | 0.001119914 | 0.011457596 | 0.922135416; 8 | SLE GWAS | 67 | Coronary
artery disease ∥ id: ebi-a-GCST005194 | Penalised weighted
median | 57 | 0.002324955 | 0.011137365 | 0.834641323; 8 | SLE GWAS | 67 | Coronary
artery disease ∥ id: ebi-a-GCST005194 | Inverse variance
weighted | 57 | 0.016028351 | 0.013517121 | 0.235708611; 8 | SLE GWAS | 67 | Coronary
artery disease ∥ id: ebi-a-GCST005194 | Inverse variance weighted (multiplicative random
effects) | 57 | 0.016028351 | 0.013517121 | 0.235708611; 8 | SLE GWAS | 67 | Coronary
artery disease ∥ id: ebi-a-GCST005194 | Inverse variance weighted (fixed
effects) | 57 | 0.016028351 | 0.006623969 | 0.015531161; 8 | SLE GWAS | 67 | Coronary
artery disease ∥ id: ebi-a-GCST005194 | Simple
mode | 57 | 0.01902842 | 0.019123299 | 0.323997753; 8 | SLE GWAS | 67 | Coronary
artery disease ∥ id: ebi-a-GCST005194 | Weighted
mode | 57 | 0.002388694 | 0.012668543 | 0.851124707; 8 | SLE GWAS | 67 | Coronary
artery disease ∥ id: ebi-a-GCST005194 | Weighted mode
(NOME) | 57 | 0.002388694 | 0.01313993 | 0.856404817; 8 | SLE GWAS | 67 | Coronary
artery disease ∥ id: ebi-a-GCST005194 | Simple mode
(NOME) | 57 | 0.01902842 | 0.01957218 | 0.335123061; 8 | SLE GWAS | 67 | Coronary
artery disease ∥ id: ebi-a-GCST005194 | Robust adjusted profile score
(RAPS) | 57 | 0.004647331 | 0.01171684 | 0.691635247; 8 | SLE GWAS | 67 | Coronary
artery disease ∥ id: ebi-a-
GCST005194 | Raw | 57 | 0.016028351 | 0.013517121 | 0.24071571; 8 | SLE
GWAS | 67 | Coronary artery disease ∥ id: ebi-a-GCST005194 | Outlier-corrected | 52
| −0.000376469 | 0.009633856 | 0.968981025; 9 | SLE GWAS | 23 | Coronary artery disease ∥
id: ebi-a-GCST005194 | Maximum likelihood | 18 | 0.055391035 | 0.013560579 | 4.41319E−05;
9 | SLE GWAS | 23 | Coronary artery disease ∥ id: ebi-a-GCST005194 | MR
Egger | 18 | −0.041461152 | 0.05086826 | 0.426999348; 9 | SLE GWAS | 23 | Coronary
artery disease ∥ id: ebi-a-GCST005194 | MR Egger (bootstrap) | 18 | −0.017984062
| 0.03139111 | 0.272; 9 | SLE GWAS | 23 | Coronary artery disease ∥ id: ebi-a-
GCST005194 | Simple median | 18 | 0.050146018 | 0.036808496 | 0.173087768; 9 | SLE
GWAS | 23 | Coronary artery disease ∥ id: ebi-a-GCST005194 | Weighted median | 18
| −0.002215633 | 0.016823028 | 0.895219682; 9 | SLE GWAS | 23 | Coronary artery disease ∥
id: ebi-a-GCST005194 | Penalised weighted median | 18 | −0.006166006 |
0.016912528 | 0.715423354; 9 | SLE GWAS | 23 | Coronary artery disease ∥
id: ebi-a-GCST005194 | Inverse variance
weighted | 18 | 0.039572721 | 0.024961382 | 0.112885071; 9 | SLE GWAS | 23 | Coronary
artery disease ∥ id: ebi-a-GCST005194 | Inverse variance weighted (multiplicative random
effects) | 18 | 0.039572721 | 0.024961382 | 0.112885071; 9 | SLE GWAS | 23 | Coronary
artery disease ∥ id: ebi-a-GCST005194 | Inverse variance weighted (fixed
effects) | 18 | 0.039572721 | 0.010932778 | 0.000295013; 9 | SLE GWAS | 23 | Coronary
artery disease ∥ id: ebi-a-GCST005194 | Simple mode | 18 | −3.77128E−05 |
0.025044221 | 0.998816037; 9 | SLE GWAS | 23 | Coronary artery disease ∥ id: ebi-a-
GCST005194 | Weighted mode | 18 | −0.005746771 | 0.015902877 | 0.72227734; 9 | SLE
GWAS | 23 | Coronary artery disease ∥ id: ebi-a-GCST005194 | Weighted mode
(NOME) | 18 | −0.005746771 | 0.013925546 | 0.685000186; 9 | SLE GWAS | 23 | Coronary
artery disease ∥ id: ebi-a-GCST005194 | Simple mode (NOME) | 18 | −3.77128E−05 |
0.023959413 | 0.998762431; 9 | SLE GWAS | 23 | Coronary artery disease ∥ id: ebi-a-
GCST005194 | Robust adjusted profile score
(RAPS) | 18 | 0.042549357 | 0.029878743 | 0.154426829; 9 | SLE GWAS | 23 | Coronary
artery disease ∥ id: ebi-a-
GCST005194 | Raw | 18 | 0.039572721 | 0.024961382 | 0.131309751; 9 | SLE
GWAS | 23 | Coronary artery disease ∥ id: ebi-a-GCST005194 | Outlier-corrected | 14
| −0.001522359 | 0.017175544 | 0.930722668; 10 | SLE GWAS | 99 | Coronary artery disease
∥ id: ebi-a-GCST005194 | Maximum likelihood | 88 | 0.001688992 | 0.0063701 | 7.91E−01;
10 | SLE GWAS | 99 | Coronary artery disease ∥ id: ebi-a-GCST005194 | MR
Egger | 88 | 0.002388639 | 0.013414804 | 0.859094676; 10 | SLE GWAS | 99 | Coronary
artery disease ∥ id: ebi-a-GCST005194 | MR Egger (bootstrap) | 88 | −0.000490223 |
0.009768488 | 0.473; 10 | SLE GWAS | 99 | Coronary artery disease ∥
id: ebi-a-GCST005194 | Simple median | 88 | −0.006130446 | 0.014830406 | 0.679335505;
10 | SLE GWAS | 99 | Coronary artery disease ∥ id: ebi-a-GCST005194 | Weighted
median | 88 | 0.007009364 | 0.009242661 | 0.448229057; 10 | SLE GWAS | 99 | Coronary
artery disease ∥ id: ebi-a-GCST005194 | Penalised weighted
median | 88 | 0.006769094 | 0.009664502 | 0.483672626; 10 | SLE GWAS | 99 | Coronary
artery disease ∥ id: ebi-a-GCST005194 | Inverse variance
weighted | 88 | 0.00155512 | 0.009497176 | 0.869931477; 10 | SLE GWAS | 99 | Coronary
artery disease ∥ id: ebi-a-GCST005194 | Inverse variance weighted (multiplicative random
effects) | 88 | 0.00155512 | 0.009497176 | 0.869931477; 10 | SLE GWAS | 99 | Coronary
artery disease ∥ id: ebi-a-GCST005194 | Inverse variance weighted (fixed
effects) | 88 | 0.00155512 | 0.005665503 | 0.783708623; 10 | SLE GWAS | 99 | Coronary
artery disease ∥ id: ebi-a-GCST005194 | Simple mode | 88
| −0.00498192 | 0.020009457 | 0.80396442; 10 | SLE GWAS | 99 | Coronary artery disease ∥
id: ebi-a-GCST005194 | Weighted mode | 88 | −0.013424588 | 0.711462119;
10 | SLE GWAS | 99 | Coronary artery disease ∥ id: ebi-a-GCST005194 | Weighted mode
(NOME) | 88 | −0.00498192 | 0.006965562 | 0.476387175; 10 | SLE GWAS | 99 | Coronary
artery disease ∥ id: ebi-a-GCST005194 | Simple mode (NOME) | 88 | −0.00498192 |
0.01660506 | 0.764874363; 10 | SLE GWAS | 99 | Coronary artery disease ∥
id: ebi-a-GCST005194 | Robust adjusted profile score (RAPS) | 88
| −0.000101238 | 0.010430293 | 0.992255763; 10 | SLE GWAS | 99 | Coronary artery disease
∥ id: ebi-a-GCST005194 | Raw | 88 | 0.00155512 | 0.009497176 | 0.870311277; 10 | SLE
GWAS | 99 | Coronary artery disease ∥ id: ebi-a-GCST005194 | Outlier-corrected | 85
| −0.001005036 | 0.008710688 | 0.908419577; 11 | SLE GWAS | 183 | Coronary artery
disease ∥ id: ebi-a-GCST005194 | Maximum
likelihood | 153 | 0.004218142 | 0.003464523 | 0.223404707; 11 | SLE
GWAS | 183 | Coronary artery disease ∥ id: ebi-a-GCST005194 | MR
Egger | 153 | 0.000593841 | 0.009383445 | 0.949622417; 11 | SLE GWAS | 183 | Coronary
artery disease ∥ id: ebi-a-GCST005194 | MR Egger (bootstrap) | 153
| −0.017179745 | 0.008887907 | 0.028; 11 | SLE GWAS | 183 | Coronary artery disease ∥
id: ebi-a-GCST005194 | Simple median | 153 | −0.008050252 | 0.006319519 | 0.202709141;
11 | SLE GWAS | 183 | Coronary artery disease ∥ id: ebi-a-GCST005194 | Weighted
median | 153 | −0.011653751 | 0.005132871 | 0.02318237; 11 | SLE
GWAS | 183 | Coronary artery disease ∥ id: ebi-a-GCST005194 | Penalised weighted
median | 153 | −0.01363292 | 0.004949462 | 0.005879542; 11 | SLE
GWAS | 183 | Coronary artery disease ∥ id: ebi-a-GCST005194 | Inverse variance
weighted | 153 | 0.00406774 | 0.005293402 | 0.442217073; 11 | SLE
GWAS | 183 | Coronary artery disease ∥ id: ebi-a-GCST005194 | Inverse variance weighted
(multiplicative random effects) | 153 | 0.00406774 | 0.005293402 | 0.442217073; 11 | SLE
GWAS | 183 | Coronary artery disease ∥ id: ebi-a-GCST005194 | Inverse variance weighted
(fixed effects) | 153 | 0.00406774 | 0.003246124 | 0.210166852; 11 | SLE
GWAS | 183 | Coronary artery disease ∥ id: ebi-a-GCST005194 | Simple mode | 153
| −0.020021608 | 0.012009239 | 0.097536984; 11 | SLE GWAS | 183 | Coronary artery
disease ∥ id: ebi-a-GCST005194 | Weighted mode | 153 | −0.020021608 |
0.010404667 | 0.05618644; 11 | SLE GWAS | 183 | Coronary artery disease
∥ id: ebi-a-GCST005194 | Weighted mode (NOME) | 153 | −0.020021608 |
0.007958922 | 0.01292131; 11 | SLE GWAS | 183 | Coronary artery disease
∥ id: ebi-a-GCST005194 | Simple mode (NOME) | 153 | −0.020021608 |
0.009306045 | 0.033019383; 11 | SLE GWAS | 183 | Coronary artery
disease ∥ id: ebi-a-GCST005194 | Robust adjusted profile score
(RAPS) | 153 | 0.007109548 | 0.005216204 | 0.172890884; 11 | SLE
GWAS | 183 | Coronary artery disease ∥ id: ebi-a-
GCST005194 | Raw | 153 | 0.00406774 | 0.005293402 | 0.443409358; 11 | SLE
GWAS | 183 | Coronary artery disease ∥ id: ebi-a-GCST005194 | Outlier-corrected | 150
| −7.75232E−05 | 0.004520642 | 0.986340914; 12 | SLE GWAS | 199 | Coronary artery
disease ∥ id: ebi-a-GCST005194 | Maximum
likelihood | 165 | 0.01775061 | 0.002950532 | 1.78702E−09; 12 | SLE
GWAS | 199 | Coronary artery disease ∥ id: ebi-a-GCST005194 | MR
Egger | 165 | 0.005099811 | 0.010293066 | 0.620942195; 12 | SLE GWAS | 199 | Coronary
artery disease ∥ id: ebi-a-GCST005194 | MR Egger
(bootstrap) | 165 | 0.036185436 | 0.007953832 | 0; 12 | SLE GWAS | 199 | Coronary artery
disease ∥ id: ebi-a-GCST005194 | Simple
median | 165 | 0.028298072 | 0.004531685 | 4.25178E−10; 12 | SLE
GWAS | 199 | Coronary artery disease ∥ id: ebi-a-GCST005194 | Weighted
median | 165 | 0.029165831 | 0.004252552 | 6.96213E−12; 12 | SLE
GWAS | 199 | Coronary artery disease ∥ id: ebi-a-GCST005194 | Penalised weighted
median | 165 | 0.029930164 | 0.004290403 | 3.03545E−12; 12 | SLE
GWAS | 199 | 0.017599506 | 0.004469683 | 8.2325E−05; 12 | SLE
weighted | 165 | 0.017599506 | 0.004469683 | 8.2325E−05; 12 | SLE
GWAS | 199 | Coronary artery disease ∥ id: ebi-a-GCST005194 | Inverse variance weighted
(multiplicative random effects) | 165 | 0.017599506 | 0.004469683 | 8.2325E−05; 12 | SLE
GWAS | 199 | Coronary artery disease ∥ id: ebi-a-GCST005194 | Inverse variance weighted
(fixed effects) | 165 | 0.017599506 | 0.002831219 | 5.09243E−10; 12 | SLE
GWAS | 199 | Coronary artery disease ∥ id: ebi-a-GCST005194 | Simple
mode | 165 | 0.033947559 | 0.008642438 | 0.000125951; 12 | SLE GWAS | 199 | Coronary
artery disease ∥ id: ebi-a-GCST005194 | Weighted
mode | 165 | 0.033947559 | 0.008226939 | 5.84859E−05; 12 | SLE GWAS | 199 | Coronary
artery disease ∥ id: ebi-a-GCST005194 | Weighted mode
(NOME) | 165 | 0.033947559 | 0.006781432 | 1.42251E−06; 12 | SLE
GWAS | 199 | Coronary artery disease ∥ id: ebi-a-GCST005194 | Simple mode
(NOME) | 165 | 0.033947559 | 0.007054243 | 3.36252E−06; 12 | SLE
GWAS | 199 | Coronary artery disease ∥ id: ebi-a-GCST005194 | Robust adjusted profile
score (RAPS) | 165 | 0.025205363 | 0.00308905 | 3.36181E−16; 12 | SLE
GWAS | 199 | Coronary artery disease ∥ id: ebi-a-
GCST005194 | Raw | 165 | 0.017599506 | 0.004469683 | 0.000121474; 12 | SLE
GWAS | 199 | Coronary artery disease ∥ id: ebi-a-GCST005194 | Outlier-
corrected | 152 | 0.02306565 | 0.003377256 | 1.94952E−10; 13 | SLE GWAS | 55 | Coronary
artery disease ∥ id: ebi-a-GCST005194 | Maximum
likelihood | 49 | 0.032262402 | 0.005371819 | 1.90318E−09; 13 | SLE
GWAS | 55 | Coronary artery disease ∥ id: ebi-a-GCST005194 | MR
Egger | 49 | 0.03386487 | 0.018951719 | 0.080403538; 13 | SLE GWAS | 55 | Coronary
artery disease ∥ id: ebi-a-GCST005194 | MR Egger
(bootstrap) | 49 | 0.037742726 | 0.012393679 | 0; 13 | SLE GWAS | 55 | Coronary artery
disease ∥ id: ebi-a-GCST005194 | Simple
median | 49 | 0.027140881 | 0.007701119 | 0.000424639; 13 | SLE GWAS | 55 | Coronary
artery disease ∥ id: ebi-a-GCST005194 | Weighted
median 49 | 0.027999969 | 0.006882521 | 4.73631E−05; 13 | SLE GWAS | 55 | Coronary
artery disease ∥ id: ebi-a-GCST005194 | Penalised weighted
median | 49 | 0.027999919 | 0.006808654 | 3.91565E−05; 13 | SLE GWAS | 55 | Coronary
artery disease ∥ id: ebi-a-GCST005194 | Inverse variance
weighted | 49 | 0.029111718 | 0.008418114 | 0.000543751; 13 | SLE GWAS | 55 | Coronary
artery disease ∥ id: ebi-a-GCST005194 | Inverse variance weighted (multiplicative random
effects) | 49 | 0.029111718 | 0.008418114 | 0.000543751; 13 | SLE GWAS | 55 | Coronary
artery disease ∥ id: ebi-a-GCST005194 | Inverse variance weighted (fixed
effects) | 49 | 0.029111718 | 0.005056901 | 8.57081E−09; 13 | SLE GWAS | 55 | Coronary
artery disease ∥ id: ebi-a-GCST005194 | Simple
mode 49 | 0.024984841 | 0.009489371 | 0.011354969; 13 | SLE GWAS | 55 | Coronary
artery disease ∥ id: ebi-a-GCST005194 | Weighted
mode 49 | 0.029404803 | 0.00925608 | 0.002603496; 13 | SLE GWAS | 55 | Coronary
artery disease ∥ id: ebi-a-GCST005194 | Weighted mode
(NOME) | 49 | 0.029404803 | 0.007478028 | 0.000270003; 13 | SLE GWAS | 55 | Coronary
artery disease ∥ id: ebi-a-GCST005194 | Simple mode
(NOME) | 49 | 0.024984841 | 0.008373353 | 0.00446591; 13 | SLE GWAS | 55 | Coronary
artery disease ∥ id: ebi-a-GCST005194 | Robust adjusted profile score
(RAPS) | 49 | 0.026797048 | 0.007893982 | 0.000687246; 13 | SLE GWAS | 55 | Coronary
artery disease ∥ id: ebi-a-
GCST005194 | Raw | 49 | 0.029111718 | 0.008418114 | 0.00114954; 13 | SLE
GWAS | 55 | Coronary artery disease ∥ id: ebi-a-GCST005194 | Outlier-
corrected 43 | 0.026021853 | 0.00505033 | 6.47966E−06; 14 | SLE GWAS | 173 | Coronary
artery disease ∥ id: ebi-a-GCST005194 | Maximum likelihood | 155
| −0.012047063 | 0.002165946 | 2.66649E−08; 14 | SLE GWAS | 173 | Coronary artery
disease ∥ id: ebi-a-GCST005194 | MR Egger | 155 | −0.012160884
| 0.006261037 | 0.053935984; 14 | SLE GWAS | 173 | Coronary artery
disease ∥ id: ebi-a-GCST005194 | MR Egger (bootstrap) | 155
| −0.014409099 | 0.006250615 | 0.007; 14 | SLE GWAS | 173 | Coronary artery disease ∥
id: ebi-a-GCST005194 | Simple median | 155 -0.007706055 | 0.00405125 | 0.057152527;
14 | SLE GWAS | 173 | Coronary artery disease ∥ id: ebi-a-GCST005194 | Weighted
median | 155 | −0.005874657 | 0.003326022 | 0.077350327; 14 | SLE
GWAS | 173 | Coronary artery disease ∥ id: ebi-a-GCST005194 | Penalised weighted
median | 155 | −0.005831025 | 0.003391878 | 0.08559364; 14 | SLE
GWAS | 173 | Coronary artery disease ∥ id: ebi-a-GCST005194 | Inverse variance
weighted | 155 | −0.011990738 | 0.003368334 | 0.000371078; 14 | SLE
GWAS | 173 | Coronary artery disease ∥ id: ebi-a-GCST005194 | Inverse variance weighted
(multiplicative random effects) | 155 | −0.011990738 | 0.003368334 | 0.000371078;
14 | SLE GWAS | 173 | Coronary artery disease ∥ id: ebi-a-GCST005194 | Inverse variance
weighted (fixed effects) | 155 | −0.011990738 | 0.002117151 | 1.48214E−08; 14 | SLE
GWAS | 173 | Coronary artery disease ∥ id: ebi-a-GCST005194 | Simple mode | 155
| −0.044811149 | 0.008465696 | 4.0722E−07; 14 | SLE GWAS | 173 | Coronary artery disease
∥ id: ebi-a-GCST005194 | Weighted mode | 155 | −0.003659637 |
0.005819181 | 0.53035137; 14 | SLE GWAS | 173 | Coronary artery disease
∥ id: ebi-a-GCST005194 | Weighted mode (NOME) | 155 | −0.003659637 |
0.004481346 | 0.415396822; 14 | SLE GWAS | 173 | Coronary artery
disease ∥ id: ebi-a-GCST005194 | Simple mode (NOME) | 155 | −0.044811149 |
0.007284923 | 6.34627E−09; 14 | SLE GWAS | 173 | Coronary artery
disease ∥ id: ebi-a-GCST005194 | Robust adjusted profile score (RAPS) | 155
| −0.012026199 | 0.003313567 | 0.000284101; 14 | SLE GWAS | 173 | Coronary artery
disease ∥ id: ebi-a-GCST005194 | Raw | 155 | −0.011990738 | 0.003368334 | 0.000493833;
14 | SLE GWAS | 173 | Coronary artery disease ∥ id: ebi-a-GCST005194 | Outlier-
corrected | 151 | −0.012925255 | 0.002956697 | 2.2942E−05; 15 | SLE GWAS | 3 | Coronary
artery disease ∥ id: ebi-a-GCST005194 | Maximum likelihood | 2 | −0.096866713
| 0.089209354 | 0.27755163; 15 | SLE GWAS | 3 | Coronary artery disease ∥
id: ebi-a-GCST005194 | Inverse variance weighted | 2 | −0.079675638
| 0.100808459 | 0.429313708; 15 | SLE GWAS | 3 | Coronary artery disease ∥
id: ebi-a-GCST005194 | Inverse variance weighted (multiplicative random effects) | 2
| −0.079675638 | 0.100808459 | 0.429313708; 15 | SLE GWAS | 3 | Coronary artery disease ∥
id: ebi-a-GCST005194 | Inverse variance weighted (fixed effects) | 2 | −0.079675638
| 0.071646971 | 0.266112909; 15 | SLE GWAS | 3 | Coronary artery disease ∥
id: ebi-a-GCST005194 | Robust adjusted profile score (RAPS) | 2 | −0.094980859
| 0.101806207 | 0.350841939; 16 | SLE GWAS | 53 | Coronary artery disease
∥ id: ebi-a-GCST005194 | Maximum
likelihood | 45 | 0.026691892 | 0.00534619 | 5.95429E−07; 16 | SLE GWAS | 53 | Coronary
artery disease ∥ id: ebi-a-GCST005194 | MR
Egger | 45 | 0.030928344 | 0.009808501 | 0.002942297; 16 | SLE GWAS | 53 | Coronary
artery disease ∥ id: ebi-a-GCST005194 | MR Egger
(bootstrap) | 45 | 0.029688774 | 0.009478703 | 0; 16 | SLE GWAS | 53 | Coronary artery
disease ∥ id: ebi-a-GCST005194 | Simple
median | 45 | 0.027152662 0.008290645 | 0.001056259; 16 | SLE GWAS | 53 | Coronary
artery disease ∥ id: ebi-a-GCST005194 | Weighted
median | 45 0.027857739 | 0.007027537 | 7.36786E−05; 16 | SLE GWAS | 53 | Coronary
artery disease ∥ id: ebi-a-GCST005194 | Penalised weighted
median | 45 | 0.027857739 | 0.006933776 | 5.87723E−05; 16 | SLE GWAS | 53 | Coronary
artery disease ∥ id: ebi-a-GCST005194 | Inverse variance
weighted | 45 | 0.026534506 | 0.005254468 | 4.42056E−07; 16 | SLE GWAS | 53 | Coronary
artery disease ∥ id: ebi-a-GCST005194 | Inverse variance weighted (multiplicative random
effects) | 45 | 0.026534506 | 0.005254468 | 4.42056E−07; 16 | SLE GWAS | 53 | Coronary
artery disease ∥ id: ebi-a-GCST005194 | Inverse variance weighted (fixed
effects) | 45 | 0.026534506 | 0.005234991 | 4.0058E−07; 16 | SLE GWAS | 53 | Coronary
artery disease ∥ id: ebi-a-GCST005194 | Simple
mode | 45 | 0.027999129 | 0.008230457 | 0.001434871; 16 | SLE GWAS | 53 | Coronary
artery disease ∥ id: ebi-a-GCST005194 | Weighted
mode | 45 | 0.024591721 | 0.007467711 | 0.001960553; 16 | SLE GWAS | 53 | Coronary
artery disease ∥ id: ebi-a-GCST005194 | Weighted mode
(NOME) | 45 | 0.024591721 | 0.006795461 | 0.000759662; 16 | SLE GWAS | 53 | Coronary
artery disease ∥ id: ebi-a-GCST005194 | Simple mode
(NOME) | 45 | 0.027999129 | 0.007833153 | 0.000866492; 16 | SLE GWAS | 53 | Coronary
artery disease ∥ id: ebi-a-GCST005194 | Robust adjusted profile score
(RAPS) | 45 | 0.027730598 | 0.005581473 | 6.7532E−07; 16 | SLE GWAS | 53 | Coronary
artery disease ∥ id: ebi-a-GCST005194 | Raw | 45 | 0.026534506 | 0.005254468 | 8.1756E−06;
16 | SLE GWAS | 53 | Coronary artery disease ∥ id: ebi-a-GCST005194 | Outlier-
corrected | NA | NA | NA | NA; 17 | SLE GWAS | 40 | Coronary artery disease ∥ id: ebi-a-
GCST005194 | Maximum likelihood | 31 | 0.125887148 | 0.028445399 | 9.61873E−06;
17 | SLE GWAS | 40 | Coronary artery disease ∥ id: ebi-a-GCST005194 | MR
Egger 31 | 0.039194482 | 0.063735138 | 0.543377669; 17 | SLE GWAS | 40 | Coronary
artery disease ∥ id: ebi-a-GCST005194 | MR Egger
(bootstrap) | 31 | 0.086891469 | 0.045306561 | 0.026; 17 | SLE GWAS | 40 | Coronary
artery disease ∥ id: ebi-a-GCST005194 | Simple
median | 31 | 0.072726798 | 0.036879013 | 0.048605323; 17 | SLE GWAS | 40 | Coronary
artery disease ∥ id: ebi-a-GCST005194 | Weighted
median | 31 | 0.047832723 | 0.032649555 | 0.142911515; 17 | SLE GWAS | 40 | Coronary
artery disease ∥ id: ebi-a-GCST005194 | Penalised weighted
median | 31 | 0.04157154 | 0.031566132 | 0.187849844; 17 | SLE GWAS | 40 | Coronary
artery disease ∥ id: ebi-a-GCST005194 | Inverse variance
weighted | 31 | 0.062049156 | 0.029228385 | 0.03376161; 17 | SLE GWAS | 40 | Coronary
artery disease ∥ id: ebi-a-GCST005194 | Inverse variance weighted (multiplicative random
effects) | 31 | 0.062049156 | 0.029228385 | 0.03376161; 17 | SLE GWAS | 40 | Coronary
artery disease ∥ id: ebi-a-GCST005194 | Inverse variance weighted (fixed
effects) | 31 | 0.062049156 | 0.01735271 | 0.000349209; 17 | SLE GWAS | 40 | Coronary
artery disease ∥ id: ebi-a-GCST005194 | Simple
mode | 31 | 0.076773891 | 0.048023493 | 0.120373721; 17 | SLE GWAS | 40 | Coronary
artery disease ∥ id: ebi-a-GCST005194 | Weighted
mode 31 | 0.03609628 | 0.036485307 | 0.330410906; 17 | SLE GWAS | 40 | Coronary
artery disease ∥ id: ebi-a-GCST005194 | Weighted mode
(NOME) | 31 | 0.07168919 | 0.042424746 | 0.101434499; 17 | SLE GWAS | 40 | Coronary
artery disease ∥ id: ebi-a-GCST005194 | Simple mode
(NOME) | 31 | 0.076773891 | 0.05540847 | 0.176085155; 17 | SLE GWAS | 40 | Coronary
artery disease ∥ id: ebi-a-GCST005194 | Robust adjusted profile score
(RAPS) | 31 | 0.080503782 | 0.042369643 | 0.057428601; 17 | SLE GWAS | 40 | Coronary
artery disease ∥ id: ebi-a-
GCST005194 | Raw | 31 | 0.062049156 | 0.029228385 | 0.042125754; 17 | SLE
GWAS | 40 | Coronary artery disease ∥ id: ebi-a-GCST005194 | Outlier-
corrected | 28 | 0.034873812 | 0.026362808 | 0.196983343; 18 | SLE
GWAS | 196 | Coronary artery disease ∥ id: ebi-a-GCST005194 | Maximum
likelihood | 173 | 0.021369097 | 0.003149087 | 1.15438E−11; 18 | SLE
GWAS | 196 | Coronary artery disease ∥ id: ebi-a-GCST005194 | MR
Egger | 173 | 0.002862544 | 0.01176624 | 0.808076613; 18 | SLE GWAS | 196 | Coronary
artery disease ∥ id: ebi-a-GCST005194 | MR Egger
(bootstrap) | 173 | 0.018463564 | 0.00752718 | 0.011; 18 | SLE GWAS | 196 | Coronary
artery disease ∥ id: ebi-a-GCST005194 | Simple
median | 173 | 0.027140881 | 0.004839235 | 2.0408E−08; 18 | SLE GWAS | 196 | Coronary
artery disease ∥ id: ebi-a-GCST005194 | Weighted
median | 173 | 0.026392268 | 0.004626719 | 1.16811E−08; 18 | SLE
GWAS | 196 | Coronary artery disease ∥ id: ebi-a-GCST005194 | Penalised weighted
median | 173 | 0.02740625 | 0.004508942 | 1.21539E−09; 18 | SLE GWAS | 196 | Coronary
artery disease ∥ id: ebi-a-GCST005194 | Inverse variance
weighted | 173 | 0.020833422 | 0.004934227 | 2.41901E−05; 18 | SLE
GWAS | 196 | Coronary artery disease ∥ id: ebi-a-GCST005194 | Inverse variance weighted
(multiplicative random effects) | 173 | 0.020833422 | 0.004934227 | 2.41901E−05; 18 | SLE
GWAS | 196 | Coronary artery disease ∥ id: ebi-a-GCST005194 | Inverse variance weighted
(fixed effects) | 173 | 0.020833422 | 0.002953143 | 1.73023E−12; 18 | SLE
GWAS | 196 | Coronary artery disease ∥ id: ebi-a-GCST005194 | Simple
mode | 173 | 0.026804821 | 0.00885364 | 0.002844924; 18 | SLE GWAS | 196 | Coronary
artery disease ∥ id: ebi-a-GCST005194 | Weighted
mode | 173 | 0.026804821 | 0.008466944 | 0.00182955; 18 | SLE GWAS | 196 | Coronary
artery disease ∥ id: ebi-a-GCST005194 | Weighted mode
(NOME) | 173 | 0.026804821 | 0.006488791 | 5.62933E−05; 18 | SLE
GWAS | 196 | Coronary artery disease ∥ id: ebi-a-GCST005194 | Simple mode
(NOME) | 173 | 0.026804821 | 0.00774473 | 0.000678712; 18 | SLE
GWAS | 196 | Coronary artery disease ∥ id: ebi-a-GCST005194 | Robust adjusted profile
score (RAPS) | 173 | 0.018347675 | 0.004466351 | 3.99137E−05; 18 | SLE
GWAS | 196 | Coronary artery disease ∥ id: ebi-a-
GCST005194 | Raw | 173 | 0.020833422 | 0.004934227 | 3.91172E−05; 18 | SLE
GWAS | 196 | Coronary artery disease ∥ id: ebi-a-GCST005194 | Outlier-
corrected | 169 | 0.014760848 | 0.003872698 | 0.000193578; 19 | SLE
GWAS | 24 | Coronary artery disease ∥ id: ebi-a-GCST005194 | Maximum likelihood | 19
| −0.126360449 | 0.011849307 | 1.5008E−26; 19 | SLE GWAS | 24 | Coronary artery disease ∥
id: ebi-a-GCST005194 | MR Egger | 19 | −0.147803239 | 0.017701546 | 2.0285E−07;
19 | SLE GWAS | 24 | Coronary artery disease ∥ id: ebi-a-GCST005194 | MR Egger
(bootstrap) | 19 | −0.079579488 | 0.091677506 | 0.197; 19 | SLE GWAS 24 | Coronary
artery disease ∥ id: ebi-a-GCST005194 | Simple median | 19 | −0.133708056
| 0.015393849 | 3.76052E−18; 19 | SLE GWAS | 24 | Coronary artery disease
∥ id: ebi-a-GCST005194 | Weighted median | 19 | −0.133709374 | 0.015669595 | 1.42544E−17;
19 | SLE GWAS | 24 | Coronary artery disease ∥ id: ebi-a-GCST005194 | Penalised
weighted median | 19 | −0.133709374 | 0.015387811 | 3.64665E−18; 19 | SLE
GWAS | 24 | Coronary artery disease ∥ id: ebi-a-GCST005194 | Inverse variance
weighted | 19 | −0.126245868 | 0.010036026 | 2.74559E−36; 19 | SLE
GWAS | 24 | Coronary artery disease ∥ id: ebi-a-GCST005194 | Inverse variance weighted
(multiplicative random effects) | 19 | −0.126245868 | 0.006129369 | 2.92707E−94; 19 | SLE
GWAS | 24 | Coronary artery disease ∥ id: ebi-a-GCST005194 | Inverse variance weighted
(fixed effects) | 19 | −0.126245868 | 0.010036026 | 2.74559E−36; 19 | SLE
GWAS | 24 | Coronary artery disease ∥ id: ebi-a-GCST005194 | Simple mode | 19
| −0.134846109 | 0.022023571 | 8.77235E−06; 19 | SLE GWAS | 24 | Coronary artery disease
∥ id: ebi-a-GCST005194 | Weighted mode | 19 | −0.134846109 | 0.021302676 | 5.77418E−06;
19 | SLE GWAS | 24 | Coronary artery disease ∥ id: ebi-a-GCST005194 | Weighted
mode (NOME) | 19 | −0.134846109 | 0.017525232 | 4.24849E−07; 19 | SLE
GWAS | 24 | Coronary artery disease ∥ id: ebi-a-GCST005194 | Simple mode
(NOME) | 19 | −0.134846109 | 0.018763429 | 1.08941E−06; 19 | SLE
GWAS | 24 | Coronary artery disease ∥ id: ebi-a-GCST005194 | Robust adjusted profile
score (RAPS) | 19 | −0.128183504 | 0.012329452 | 2.5713E−25; 19 | SLE
GWAS | 24 | Coronary artery disease ∥ id: ebi-a-GCST005194 | Raw | 19
| −0.126245868 | 0.006129369 | 5.79412E−14; 19 | SLE GWAS | 24 | Coronary artery disease
∥ id: ebi-a-GCST005194 | Outlier-corrected | NA | NA | NA | NA; 20 | SLE
GWAS | 2 | Coronary artery disease ∥ id: ebi-a-GCST005194 | Maximum
likelihood | 2 | 0.577575085 | 0.524644651 | 0.270945336; 20 | SLE GWAS | 2 | Coronary
artery disease ∥ id: ebi-a-GCST005194 | Inverse variance
weighted | 2 | 0.275873213 | 0.320243194 | 0.388990741; 20 | SLE GWAS | 2 | Coronary
artery disease ∥ id: ebi-a-GCST005194 | Inverse variance weighted (multiplicative random
effects) | 2 | 0.275873213 | 0.320243194 | 0.388990741; 20 | SLE GWAS | 2 | Coronary
artery disease ∥ id: ebi-a-GCST005194 | Inverse variance weighted (fixed
effects) | 2 | 0.275873213 | 0.132951808 | 0.037987782; 20 | SLE GWAS | 2 | Coronary
artery disease ∥ id: ebi-a-GCST005194 | Robust adjusted profile score
(RAPS) | 2 | 0.577653475 | 0.580385456 | 0.319593871; 21 | SLE GWAS | 222 | Coronary
artery disease ∥ id: ebi-a-GCST005194 | Maximum likelihood | 196
| −0.008714533 | 0.002087058 | 2.97318E−05; 21 | SLE GWAS | 222 | Coronary artery
disease ∥ id: ebi-a-GCST005194 | MR Egger | 196 | −0.007619203
| 0.005725254 | 0.184814809; 21 | SLE GWAS | 222 | Coronary artery
disease ∥ id: ebi-a-GCST005194 | MR Egger (bootstrap) | 196 | −1.22E−02
| 0.005220978 | 0.009; 21 | SLE GWAS | 222 | Coronary artery disease ∥ id: ebi-a-
GCST005194 | Simple median | 196 | −0.008253392 | 0.004126435 | 0.04548661; 21 | SLE
GWAS | 222 | Coronary artery disease ∥ id: ebi-a-GCST005194 | Weighted median | 196
| −0.005042651 | 0.003102417 | 0.104078579; 21 | SLE GWAS | 222 | Coronary artery
disease ∥ id: ebi-a-GCST005194 | Penalised weighted median | 196
| −0.004086783 | 0.003021779 | 0.176233666; 21 | SLE GWAS | 222 | Coronary artery
disease ∥ id: ebi-a-GCST005194 | Inverse variance weighted | 196
| −0.00853801 | 0.003661643 | 0.019714205; 21 | SLE GWAS | 222 | Coronary artery disease
∥ id: ebi-a-GCST005194 | Inverse variance weighted (multiplicative random
effects) | 196 | −0.00853801 | 0.003661643 | 0.019714205; 21 | SLE
GWAS | 222 | Coronary artery disease ∥ id: ebi-a-GCST005194 | Inverse variance weighted
(fixed effects) | 196 | −0.00853801 | 0.002006598 | 2.09078E−05; 21 | SLE
GWAS | 222 | Coronary artery disease ∥ id: ebi-a-GCST005194 | Simple mode | 196
| −0.008084724 | 0.010630739 | 0.447871697; 21 | SLE GWAS | 222 | Coronary artery
disease ∥ id: ebi-a-GCST005194 | Weighted mode | 196
| −0.001886697 | 0.010320649 | 0.855138574; 21 | SLE GWAS | 222 | Coronary artery
disease ∥ id: ebi-a-GCST005194 | Weighted mode (NOME) | 196
| −0.001886697 | 0.003734614 | 0.613994422; 21 | SLE GWAS | 222 | Coronary artery
disease ∥ id: ebi-a-GCST005194 | Simple mode (NOME) | 196
| −0.008084724 | 0.00539689 | 0.135741504; 21 | SLE GWAS | 222 | Coronary artery disease
∥ id: ebi-a-GCST005194 | Robust adjusted profile score (RAPS) | 196
| −0.008824297 | 0.003162706 | 0.005269016; 21 | SLE GWAS | 222 | Coronary artery
disease ∥ id: ebi-a-GCST005194 | Raw | 196 −0.00853801 | 0.003661643 | 0.020735262;
21 | SLE GWAS | 222 | Coronary artery disease ∥ id: ebi-a-GCST005194 | Outlier-
corrected | 190 | −0.0112021 | 0.002733433 | 6.17536E−05; 22 | SLE GWAS | 48 | Coronary
artery disease ∥ id: ebi-a-GCST005194 | Maximum likelihood | 42
| −0.060244435 | 0.006313771 | 1.40439E−21; 22 | SLE GWAS | 48 | Coronary artery disease
∥ id: ebi-a-GCST005194 | MR Egger | 42 | −0.144539904 | 0.025653356 | 1.54278E−06;
22 | SLE GWAS | 48 | Coronary artery disease ∥ id: ebi-a-GCST005194 | MR Egger
(bootstrap) | 42 | −0.094451907 | 0.022351236 | 0; 22 | SLE GWAS | 48 | Coronary artery
disease ∥ id: ebi-a-GCST005194 | Simple median | 42 | −0.063864775
| 0.008131036 | 4.01546E−15; 22 | SLE GWAS | 48 | Coronary artery disease
∥ id: ebi-a-GCST005194 | Weighted median | 42 | −0.064185466 | 0.007801344 | 1.9118E−16;
22 | SLE GWAS | 48 | Coronary artery disease ∥ id: ebi-a-GCST005194 | Penalised
weighted median | 42 | −0.064437364 | 0.008043931 | 1.14075E−15; 22 | SLE
GWAS | 48 | Coronary artery disease ∥ id: ebi-a-GCST005194 | Inverse variance
weighted | 42 | −0.056140228 | 0.008482171 | 3.62579E−11; 22 | SLE
GWAS | 48 | Coronary artery disease ∥ id: ebi-a-GCST005194 | Inverse variance weighted
(multiplicative random effects) | 42 | −0.056140228 | 0.008482171 | 3.62579E−11; 22 | SLE
GWAS | 48 | Coronary artery disease ∥ id: ebi-a-GCST005194 | Inverse variance weighted
(fixed effects) | 42 | −0.056140228 | 0.005718613 | 9.50328E−23; 22 | SLE
GWAS | 48 | Coronary artery disease ∥ id: ebi-a-GCST005194 | Simple mode | 42
| −0.069979573 | 0.01162 4.02132E−07; 22 | SLE GWAS | 48 | Coronary artery disease ∥
id: ebi-a-GCST005194 | Weighted mode | 42 | −0.069979573 | 0.011019585 | 1.37265E−07;
22 | SLE GWAS | 48 | Coronary artery disease ∥ id: ebi-a-GCST005194 | Weighted mode
(NOME) | 42 | −0.069979573 | 0.010048371 | 1.8508E−08; 22 | SLE GWAS | 48 | Coronary
artery disease ∥ id: ebi-a-GCST005194 | Simple mode (NOME) | 42
| −0.069979573 | 0.010631874 | 6.43484E−08; 22 | SLE GWAS | 48 | Coronary artery disease
∥ id: ebi-a-GCST005194 | Robust adjusted profile score (RAPS) | 42
| −0.062588372 | 0.007712504 | 4.8506E−16; 22 | SLE GWAS | 48 | Coronary artery disease ∥
id: ebi-a-GCST005194 | Raw | 42 | −0.056140228 | 0.008482171 | 5.71026E−08; 22 | SLE
GWAS | 48 | Coronary artery disease ∥ id: ebi-a-GCST005194 | Outlier-corrected | 40
| −0.05812076 | 0.007607433 | 2.88023E−09; 23 | SLE GWAS | 12 | Coronary artery disease ∥
id: ebi-a-GCST005194 | Maximum
likelihood | 12 | 0.007520114 | 0.01084069 | 0.487874556; 23 | SLE GWAS | 12 | Coronary
artery disease ∥ id: ebi-a-GCST005194 | MR
Egger | 12 | 0.049920632 | 0.017908439 | 0.019200383; 23 | SLE GWAS | 12 | Coronary
artery disease ∥ id: ebi-a-GCST005194 | MR Egger
(bootstrap) | 12 | 0.047226527 | 0.018289205 | 0.008; 23 | SLE GWAS | 12 | Coronary
artery disease ∥ id: ebi-a-GCST005194 | Simple median | 12
| −0.020758396 | 0.036763963 | 0.572318806; 23 | SLE GWAS | 12 | Coronary artery disease
∥ id: ebi-a-GCST005194 | Weighted median | 12 | 0.017030482 | 0.01228098 | 0.16552215;
23 | SLE GWAS | 12 | Coronary artery disease ∥ id: ebi-a-GCST005194 | Penalised
weighted median | 12 | 0.017030482 | 0.012214489 | 0.163231485; 23 | SLE
GWAS | 12 | Coronary artery disease ∥ id: ebi-a-GCST005194 | Inverse variance
weighted | 12 | 0.007308402 | 0.015748499 | 0.642597775; 23 | SLE GWAS | 12 | Coronary
artery disease ∥ id: ebi-a-GCST005194 | Inverse variance weighted (multiplicative random
effects) | 12 | 0.007308402 | 0.015748499 | 0.642597775; 23 | SLE GWAS | 12 | Coronary
artery disease ∥ id: ebi-a-GCST005194 | Inverse variance weighted (fixed
effects) | 12 | 0.007308402 | 0.010457349 | 0.48462883; 23 | SLE GWAS | 12 | Coronary
artery disease ∥ id: ebi-a-GCST005194 | Simple
mode | 12 | 0.024592024 | 0.02209693 | 0.289473955; 23 | SLE GWAS | 12 | Coronary
artery disease ∥ id: ebi-a-GCST005194 | Weighted
mode | 12 | 0.020591704 | 0.011653271 | 0.104917831; 23 | SLE GWAS | 12 | Coronary
artery disease ∥ id: ebi-a-GCST005194 | Weighted mode
(NOME) | 12 | 0.020591704 | 0.011170847 | 0.092365005; 23 | SLE GWAS | 12 | Coronary
artery disease ∥ id: ebi-a-GCST005194 | Simple mode
(NOME) | 12 | 0.024592024 | 0.022271227 | 0.29307246; 23 | SLE GWAS | 12 | Coronary
artery disease ∥ id: ebi-a-GCST005194 | Robust adjusted profile score
(RAPS) | 12 | 0.008882621 | 0.016235536 | 0.584303296; 23 | SLE GWAS | 12 | Coronary
artery disease ∥ id: ebi-a-
GCST005194 | Raw | 12 | 0.007308402 | 0.015748499 | 0.651646666; 23 | SLE
GWAS | 12 | Coronary artery disease ∥ id: ebi-a-GCST005194 | Outlier-
corrected | NA | NA | NA | NA; 24 | SLE GWAS | 17 | Coronary artery disease ∥ id: ebi-a-
GCST005194 | Maximum likelihood | 14 | −0.00616578 | 0.031520063 | 8.45E−01; 24 | SLE
GWAS | 17 | Coronary artery disease ∥ id: ebi-a-GCST005194 | MR Egger | 14
| −0.023608569 | 0.080987568 | 0.775640145; 24 | SLE GWAS | 17 | Coronary artery disease
∥ id: ebi-a-GCST005194 | MR Egger (bootstrap) | 14 -0.015796381 | 0.057596639 | 0.377;
24 | SLE GWAS | 17 | Coronary artery disease ∥ id: ebi-a-GCST005194 | Simple
median | 14 | −0.018577879 | 0.047360109 | 0.694859839; 24 | SLE GWAS | 17 | Coronary
artery disease ∥ id: ebi-a-GCST005194 | Weighted
median | 14 | 0.006170169 | 0.037778182 | 0.870261554; 24 | SLE GWAS | 17 | Coronary
artery disease ∥ id: ebi-a-GCST005194 | Penalised weighted
median | 14 | 0.006170169 | 0.038230175 | 0.871782091; 24 | SLE GWAS | 17 | Coronary
artery disease ∥ id: ebi-a-GCST005194 | Inverse variance weighted | 14
| −0.005128747 | 0.038264899 | 8.93E−01; 24 | SLE GWAS | 17 | Coronary artery disease ∥
id: ebi-a-GCST005194 | Inverse variance weighted (multiplicative random effects) | 14
| −0.005128747 | 0.038264899 | 8.93E−01; 24 | SLE GWAS | 17 | Coronary artery disease ∥
id: ebi-a-GCST005194 | Inverse variance weighted (fixed effects) | 14
| −0.005128747 | 0.023218564 | 8.25E−01; 24 | SLE GWAS | 17 | Coronary artery disease ∥
id: ebi-a-GCST005194 | Simple mode | 14 | −0.049490138 | 0.051833748 | 0.357104099;
24 | SLE GWAS | 17 | Coronary artery disease ∥ id: ebi-a-GCST005194 | Weighted
mode | 14 | 0.000405125 | 0.031240495 | 0.989850267; 24 | SLE GWAS | 17 | Coronary
artery disease ∥ id: ebi-a-GCST005194 | Weighted mode (NOME) | 14
| −0.003158822 | 0.033869377 | 0.927115206; 24 | SLE GWAS | 17 | Coronary artery disease
∥ id: ebi-a-GCST005194 | Simple mode (NOME) | 14
| −0.049490138 | 0.056784458 | 0.399267296; 24 | SLE GWAS | 17 | Coronary artery disease
∥ id: ebi-a-GCST005194 | Robust adjusted profile score (RAPS) | 14
| −0.001999408 | 0.050851343 | 9.69E−01; 24 | SLE GWAS | 17 | Coronary artery disease ∥
id: ebi-a-GCST005194 | Raw | 14 | −0.005128747 | 0.038264899 | 0.895429503; 24 | SLE
GWAS | 17 | Coronary artery disease ∥ id: ebi-a-GCST005194 | Outlier-corrected | 12
| −0.032104907 | 0.030603788 | 0.31665742; 25 | SLE GWAS | 10 | Coronary artery disease ∥
id: ebi-a-GCST005194 | Maximum likelihood | 7 | −0.388643999
| 0.180072084 | 0.030906923; 25 | SLE GWAS | 10 | Coronary artery disease
∥ id: ebi-a-GCST005194 | MR Egger | 7 | −0.08877765 | 0.243003529 | 0.729807068;
25 | SLE GWAS | 10 | Coronary artery disease ∥ id: ebi-a-GCST005194 | MR Egger
(bootstrap) | 7 | −0.111368427 | 0.196730269 | 0.27; 25 | SLE GWAS | 10 | Coronary artery
disease ∥ id: ebi-a-GCST005194 | Simple
median | 7 | 0.107937471 | 0.179476386 | 0.547572217; 25 | SLE GWAS | 10 | Coronary
artery disease ∥ id: ebi-a-GCST005194 | Weighted median | 7 | −0.00955557
| 0.369865518 | 0.979388742; 25 | SLE GWAS | 10 | Coronary artery disease ∥
id: ebi-a-GCST005194 | Penalised weighted median | 7 | −0.00955557
| 1.215384845 | 0.993726956; 25 | SLE GWAS | 10 | Coronary artery disease ∥
id: ebi-a-GCST005194 | Inverse variance weighted | 7 | −0.041843218
| 0.113752499 | 0.712989269; 25 | SLE GWAS | 10 | Coronary artery disease
∥ id: ebi-a-GCST005194 | Inverse variance weighted (multiplicative random effects) | 7
| −0.041843218 | 0.113752499 | 0.712989269; 25 | SLE GWAS | 10 | Coronary artery disease
∥ id: ebi-a-GCST005194 | Inverse variance weighted (fixed effects) | 7
| −0.041843218 | 0.0556926 | 0.452457347; 25 | SLE GWAS | 10 | Coronary artery disease ∥
id: ebi-a-GCST005194 | Simple mode | 7 | −0.186314706 | 0.228110439 | 0.445268642;
25 | SLE GWAS | 10 | Coronary artery disease ∥ id: ebi-a-GCST005194 | Weighted
mode | 7 | 0.028088458 | 0.103730972 | 0.795633036; 25 | SLE GWAS | 10 | Coronary
artery disease ∥ id: ebi-a-GCST005194 | Weighted mode (NOME) | 7 | −0.074240325
| 0.088262302 | 0.432511235; 25 | SLE GWAS | 10 | Coronary artery disease
∥ id: ebi-a-GCST005194 | Simple mode (NOME) | 7 | −0.186314706
| 0.278790391 | 0.528800448; 25 | SLE GWAS | 10 | Coronary artery disease
∥ id: ebi-a-GCST005194 | Robust adjusted profile score (RAPS) | 7 | −0.015616241
| 0.216797804 | 0.942576951; 25 | SLE GWAS | 10 | Coronary artery disease
∥ id: ebi-a-GCST005194 | Raw | 7 | −0.041843218 | 0.113752499 | 0.725616946; 25 | SLE
GWAS | 10 | Coronary artery disease ∥ id: ebi-a-GCST005194 | Outlier-
corrected | 6 | 0.090729923 | 0.127370221 | 0.50810941; 26 | SLE GWAS | 38 | Coronary
artery disease ∥ id: ebi-a-GCST005194 | Maximum
likelihood | 30 | 0.030095761 | 0.011820523 | 0.010894643; 26 | SLE
GWAS | 38 | Coronary artery disease ∥ id: ebi-a-GCST005194 | MR
Egger | 30 | 0.022615663 | 0.027952588 | 0.425295557; 26 | SLE GWAS | 38 | Coronary
artery disease ∥ id: ebi-a-GCST005194 | MR Egger
(bootstrap) | 30 | 0.067932496 | 0.021956 | 0.004; 26 | SLE GWAS | 38 | Coronary artery
disease ∥ id: ebi-a-GCST005194 | Simple
median | 30 | 0.012516502 | 0.021805107 | 0.565956284; 26 | SLE GWAS | 38 | Coronary
artery disease ∥ id: ebi-a-GCST005194 | Weighted
median | 30 | 0.018923255 | 0.016448582 | 0.249959; 26 | SLE GWAS | 38 | Coronary
artery disease ∥ id: ebi-a-GCST005194 | Penalised weighted
median | 30 | 0.01754646 | 0.016079339 | 0.275166135; 26 | SLE GWAS | 38 | Coronary
artery disease ∥ id: ebi-a-GCST005194 | Inverse variance
weighted | 30 | 0.025794359 | 0.015992023 | 0.106755025; 26 | SLE GWAS | 38 | Coronary
artery disease ∥ id: ebi-a-GCST005194 | Inverse variance weighted (multiplicative random
effects) | 30 | 0.025794359 | 0.015992023 | 0.106755025; 26 | SLE GWAS | 38 | Coronary
artery disease ∥ id: ebi-a-GCST005194 | Inverse variance weighted (fixed
effects) | 30 | 0.025794359 | 0.010603386 | 0.014988655; 26 | SLE GWAS | 38 | Coronary
artery disease ∥ id: ebi-a-GCST005194 | Simple
mode | 30 | 0.047917991 | 0.028229301 | 0.100318834; 26 | SLE GWAS | 38 | Coronary
artery disease ∥ id: ebi-a-GCST005194 | Weighted
mode | 30 | 0.028763722 | 0.015251922 | 0.069359676; 26 | SLE GWAS | 38 | Coronary
artery disease ∥ id: ebi-a-GCST005194 | Weighted mode
(NOME) | 30 | 0.032594576 | 0.015110568 | 0.039423619; 26 | SLE GWAS | 38 | Coronary
artery disease ∥ id: ebi-a-GCST005194 | Simple mode
(NOME) | 30 | 0.047917991 | 0.027258433 | 0.089314251; 26 | SLE GWAS | 38 | Coronary
artery disease ∥ id: ebi-a-GCST005194 | Robust adjusted profile score
(RAPS) | 30 0.015605897 | 0.017718872 | 0.378453149; 26 | SLE GWAS | 38 | Coronary
artery disease ∥ id: ebi-a-
GCST005194 | Raw | 30 | 0.025794359 | 0.015992023 | 0.11758589; 26 | SLE
GWAS | 38 | Coronary artery disease ∥ id: ebi-a-GCST005194 | Outlier-
corrected | 29 | 0.015223914 | 0.014845741 | 0.313918042; 27 | SLE GWAS | 9 | Coronary
artery disease ∥ id: ebi-a-GCST005194 | Maximum
likelihood | 7 | 0.221162227 | 0.060946616 | 0.000284757; 27 | SLE GWAS | 9 | Coronary
artery disease ∥ id: ebi-a-GCST005194 | MR
Egger | 7 | 0.435305801 | 0.324653526 | 0.237663568; 27 | SLE GWAS | 9 | Coronary
artery disease ∥ id: ebi-a-GCST005194 | MR Egger
(bootstrap) | 7 | 0.083267467 | 0.080822612 | 0.145; 27 | SLE GWAS | 9 | Coronary artery
disease ∥ id: ebi-a-GCST005194 | Simple median | 7 | −0.011697435
| 0.096122101 | 0.903141757; 27 | SLE GWAS | 9 | Coronary artery disease ∥
id: ebi-a-GCST005194 | Weighted median | 7 | 0.090281425 | 0.053866546 | 0.093734215;
27 | SLE GWAS | 9 | Coronary artery disease ∥ id: ebi-a-GCST005194 | Penalised weighted
median | 7 | 0.138881216 | 0.051786256 | 0.007322369; 27 | SLE GWAS | 9 | Coronary
artery disease ∥ id: ebi-a-GCST005194 | Inverse variance
weighted | 7 | 0.094322108 | 0.067436085 | 0.161906277; 27 | SLE GWAS 9 | Coronary
artery disease ∥ id: ebi-a-GCST005194 | Inverse variance weighted (multiplicative random
effects) | 7 | 0.094322108 | 0.067436085 | 0.161906277; 27 | SLE GWAS | 9 | Coronary
artery disease ∥ id: ebi-a-GCST005194 | Inverse variance weighted (fixed
effects) | 7 | 0.094322108 | 0.026365791 | 0.000346972; 27 | SLE GWAS | 9 | Coronary
artery disease ∥ id: ebi-a-GCST005194 | Simple mode | 7 | −0.046444117
| 0.118900905 | 0.709575988; 27 | SLE GWAS | 9 | Coronary artery disease ∥
id: ebi-a-GCST005194 | Weighted mode | 7 | 0.110409199 | 0.051602199 | 0.076190133;
27 | SLE GWAS | 9 | Coronary artery disease ∥ id: ebi-a-GCST005194 | Weighted mode
(NOME) | 7 | 0.170162843 | 0.048466645 | 0.012657159; 27 | SLE GWAS | 9 | Coronary
artery disease ∥ id: ebi-a-GCST005194 | Simple mode (NOME) | 7 | −0.046444117
| 0.153986966 | 0.773129213; 27 | SLE GWAS | 9 | Coronary artery disease ∥
id: ebi-a-GCST005194 | Robust adjusted profile score
(RAPS) | 7 | 0.110589451 | 0.093593479 | 0.237367012; 27 | SLE GWAS | 9 | Coronary
artery disease ∥ id: ebi-a-
GCST005194 | Raw | 7 | 0.094322108 | 0.067436085 | 0.211414852; 27 | SLE
GWAS | 9 | Coronary artery disease ∥ id: ebi-a-GCST005194 | Outlier-
corrected | 4 | 0.061058641 | 0.052423942 | 0.328333875; 28 | SLE GWAS | 11 | Coronary
artery disease ∥ id: ebi-a-GCST005194 | Maximum
likelihood | 8 | 0.154905589 | 0.045218174 | 0.000613133; 28 | SLE GWAS | 11 | Coronary
artery disease ∥ id: ebi-a-GCST005194 | MR
Egger | 8 | 0.102948516 | 0.131928919 | 0.464856283; 28 | SLE GWAS | 11 | Coronary
artery disease ∥ id: ebi-a-GCST005194 | MR Egger
(bootstrap) | 8 | 0.11835666 | 0.06090723 | 0.024; 28 | SLE GWAS | 11 | Coronary artery
disease ∥ id: ebi-a-GCST005194 | Simple median | 8 | −0.020569631
| 0.075783637 | 0.786063594; 28 | SLE GWAS | 11 | Coronary artery disease
∥ id: ebi-a-GCST005194 | Weighted median | 8 | 0.029940269 | 0.039630117 | 0.44995334;
28 | SLE GWAS | 11 | Coronary artery disease ∥ id: ebi-a-GCST005194 | Penalised
weighted median | 8 | 0.014706425 | 0.039845566 | 0.712063911; 28 | SLE
GWAS | 11 | Coronary artery disease ∥ id: ebi-a-GCST005194 | Inverse variance
weighted | 8 | 0.080547858 | 0.060651697 | 0.184165017; 28 | SLE GWAS | 11 | Coronary
artery disease ∥ id: ebi-a-GCST005194 | Inverse variance weighted (multiplicative random
effects) | 8 | 0.080547858 | 0.060651697 | 0.184165017; 28 | SLE GWAS | 11 | Coronary
artery disease ∥ id: ebi-a-GCST005194 | Inverse variance weighted (fixed
effects) | 8 | 0.080547858 | 0.025811477 | 0.001804696; 28 | SLE GWAS | 11 | Coronary
artery disease ∥ id: ebi-a-GCST005194 | Simple
mode | 8 | 0.10167178 | 0.094844738 | 0.319289299; 28 | SLE GWAS | 11 | Coronary
artery disease ∥ id: ebi-a-GCST005194 | Weighted
mode | 8 | 0.026862313 | 0.037716094 | 0.499369772; 28 | SLE GWAS | 11 | Coronary
artery disease ∥ id: ebi-a-GCST005194 | Weighted mode
(NOME) | 8 | 0.068755615 | 0.043981563 | 0.16196071; 28 | SLE GWAS | 11 | Coronary
artery disease ∥ id: ebi-a-GCST005194 | Simple mode
(NOME) | 8 | 0.10167178 | 0.12471532 | 0.441790816; 28 | SLE GWAS | 11 | Coronary
artery disease ∥ id: ebi-a-GCST005194 | Robust adjusted profile score
(RAPS) | 8 | 0.070939296 | 0.085330738 | 0.405778668; 28 | SLE GWAS | 11 | Coronary
artery disease ∥ id: ebi-a-
GCST005194 | Raw | 8 | 0.080547858 | 0.060651697 | 0.225829259; 28 | SLE
GWAS | 11 | Coronary artery disease ∥ id: ebi-a-GCST005194 | Outlier-corrected | 5
| −0.000108179 | 0.032452013 | 0.997499883; 29 | SLE GWAS | 15 | Coronary artery disease
∥ id: ebi-a-GCST005194 | Maximum
likelihood | 13 | 0.037338233 | 0.020703916 | 0.071319422; 29 | SLE
GWAS | 15 | Coronary artery disease ∥ id: ebi-a-GCST005194 | MR
Egger | 13 | 0.031636967 | 0.06452988 | 0.633576268; 29 | SLE GWAS | 15 | Coronary
artery disease ∥ id: ebi-a-GCST005194 | MR Egger
(bootstrap) | 13 | 0.045584458 | 0.046900041 | 0.159; 29 | SLE GWAS | 15 | Coronary
artery disease ∥ id: ebi-a-GCST005194 | Simple
median | 13 | 0.029009826 | 0.037472301 | 0.438831924; 29 | SLE GWAS | 15 | Coronary
artery disease ∥ id: ebi-a-GCST005194 | Weighted
median | 13 | 0.060826686 | 0.0254044 | 0.016650459; 29 | SLE GWAS | 15 | Coronary
artery disease ∥ id: ebi-a-GCST005194 | Penalised weighted
median | 13 | 0.063555272 | 0.025592802 | 0.01301619; 29 | SLE GWAS | 15 | Coronary
artery disease ∥ id: ebi-a-GCST005194 | Inverse variance
weighted | 13 | 0.026480369 | 0.033376593 | 0.427555621; 29 | SLE GWAS | 15 | Coronary
artery disease ∥ id: ebi-a-GCST005194 | Inverse variance weighted (multiplicative random
effects) | 13 | 0.026480369 | 0.033376593 | 0.427555621; 29 | SLE GWAS | 15 | Coronary
artery disease ∥ id: ebi-a-GCST005194 | Inverse variance weighted (fixed
effects) | 13 | 0.026480369 | 0.016693548 | 0.112679493; 29 | SLE GWAS | 15 | Coronary
artery disease ∥ id: ebi-a-GCST005194 | Simple
mode | 13 0.050251972 | 0.044658025 | 0.28248137; 29 | SLE GWAS | 15 | Coronary
artery disease ∥ id: ebi-a-GCST005194 | Weighted
mode | 13 | 0.054523933 | 0.024023085 | 0.042465019; 29 | SLE GWAS | 15 | Coronary
artery disease ∥ id: ebi-a-GCST005194 | Weighted mode
(NOME) | 13 | 0.058795894 | 0.021734837 | 0.019126429; 29 | SLE GWAS | 15 | Coronary
artery disease ∥ id: ebi-a-GCST005194 | Simple mode
(NOME) | 13 | 0.050251972 | 0.047910213 | 0.314900325; 29 | SLE GWAS | 15 | Coronary
artery disease ∥ id: ebi-a-GCST005194 | Robust adjusted profile score
(RAPS) | 13 | 0.015848639 | 0.034470354 | 0.645677061; 29 | SLE GWAS | 15 | Coronary
artery disease ∥ id: ebi-a-
GCST005194 | Raw | 13 | 0.026480369 | 0.033376593 | 0.442960948; 29 | SLE
GWAS | 15 | Coronary artery disease ∥ id: ebi-a-GCST005194 | Outlier-corrected | 10
| −0.005091009 0.029735594 | 0.867846722; 30 | SLE GWAS | 10 | Coronary artery disease
∥ id: ebi-a-GCST005194 | Maximum likelihood | 6 | 0.013646927 | 0.027204808 | 6.16E−01;
30 | SLE GWAS | 10 | Coronary artery disease ∥ id: ebi-a-GCST005194 | MR Egger | 6
| −0.054647874 | 0.061050528 | 0.42131643; 30 | SLE GWAS | 10 | Coronary artery disease ∥
id: ebi-a-GCST005194 | MR Egger (bootstrap) | 6 | −0.060222325 | 0.057157085 | 0.137;
30 | SLE GWAS | 10 | Coronary artery disease ∥ id: ebi-a-GCST005194 | Simple
median | 6 | 0.05418221 | 0.045040773 | 2.29E−01; 30 | SLE GWAS | 10 | Coronary artery
disease ∥ id: ebi-a-GCST005194 | Weighted
median | 6 | 0.005549205 | 0.032724164 | 8.65E−01; 30 | SLE GWAS | 10 | Coronary artery
disease ∥ id: ebi-a-GCST005194 | Penalised weighted
median | 6 | 0.005549205 | 0.032019373 | 8.62E−01; 30 | SLE GWAS | 10 | Coronary artery
disease ∥ id: ebi-a-GCST005194 | Inverse variance
weighted | 6 | 0.012375875 | 0.025902811 | 0.63280598; 30 | SLE GWAS | 10 | Coronary
artery disease ∥ id: ebi-a-GCST005194 | Inverse variance weighted (multiplicative random
effects) | 6 | 0.012375875 | 0.024740249 | 0.616911428; 30 | SLE GWAS | 10 | Coronary
artery disease ∥ id: ebi-a-GCST005194 | Inverse variance weighted (fixed
effects) | 6 | 0.012375875 | 0.025902811 | 6.33E−01; 30 | SLE GWAS | 10 | Coronary artery
disease ∥ id: ebi-a-GCST005194 | Simple
mode | 6 | 0.054912025 | 0.056495332 | 0.375703771; 30 | SLE GWAS | 10 | Coronary
artery disease ∥ id: ebi-a-GCST005194 | Weighted mode | 6 | −0.033099236
| 0.035247354 | 3.91E−01; 30 | SLE GWAS | 10 | Coronary artery disease ∥
id: ebi-a-GCST005194 | Weighted mode (NOME) | 6 | −0.033099236
| 0.034259509 | 3.78E−01; 30 | SLE GWAS | 10 | Coronary artery disease ∥
id: ebi-a-GCST005194 | Simple mode
(NOME) | 6 | 0.054912025 | 0.053468548 | 0.351515792; 30 | SLE GWAS | 10 | Coronary
artery disease ∥ id: ebi-a-GCST005194 | Robust adjusted profile score
(RAPS) | 6 | 0.013652578 | 0.030189436 | 6.51E−01; 30 | SLE GWAS | 10 | Coronary
artery disease ∥ id: ebi-a-
GCST005194 | Raw | 6 | 0.012375875 | 0.024740249 | 0.638146447; 30 | SLE
GWAS | 10 | Coronary artery disease ∥ id: ebi-a-GCST005194 | Outlier-
corrected | NA NA NA | NA; 31 | SLE GWAS | 14 | Coronary artery disease ∥ id: ebi-a-
GCST005194 | Maximum likelihood | 11 | 0.106077517 | 0.032616939 | 0.001145067;
31 | SLE GWAS | 14 | Coronary artery disease ∥ id: ebi-a-GCST005194 | MR
Egger | 11 | 0.05374609 | 0.074211386 | 0.487323008; 31 | SLE GWAS | 14 | Coronary
artery disease ∥ id: ebi-a-GCST005194 | MR Egger
(bootstrap) | 11 | 0.022223599 | 0.065314432 | 0.362; 31 | SLE GWAS | 14 | Coronary
artery disease ∥ id: ebi-a-GCST005194 | Simple
median | 11 | 0.109835592 | 0.057132027 | 0.054544481; 31 | SLE GWAS | 14 | Coronary
artery disease ∥ id: ebi-a-GCST005194 | Weighted median | 11 | −0.002091136
| 0.042698319 | 0.960939474; 31 | SLE GWAS | 14 | Coronary artery disease
∥ id: ebi-a-GCST005194 | Penalised weighted median | 11 | −0.006360125
| 0.042721042 | 0.881651709; 31 | SLE GWAS | 14 | Coronary artery disease
∥ id: ebi-a-GCST005194 | Inverse variance
weighted | 11 | 0.083799516 | 0.03222313 | 0.009306052; 31 | SLE GWAS | 14 | Coronary
artery disease ∥ id: ebi-a-GCST005194 | Inverse variance weighted (multiplicative random
effects) | 11 | 0.083799516 | 0.03222313 | 0.009306052; 31 | SLE GWAS | 14 | Coronary
artery disease ∥ id: ebi-a-GCST005194 | Inverse variance weighted (fixed
effects) | 11 | 0.083799516 | 0.025539982 | 0.001033991; 31 | SLE GWAS | 14 | Coronary
artery disease ∥ id: ebi-a-GCST005194 | Simple
mode | 11 | 0.024187619 | 0.056254855 | 0.676339022; 31 | SLE GWAS | 14 | Coronary
artery disease ∥ id: ebi-a-GCST005194 | Weighted
mode | 11 | 0.002484934 | 0.039663386 | 0.951279332; 31 | SLE GWAS | 14 | Coronary
artery disease ∥ id: ebi-a-GCST005194 | Weighted mode
(NOME) | 11 | 0.007135509 | 0.061207275 | 0.909501385; 31 | SLE GWAS | 14 | Coronary
artery disease ∥ id: ebi-a-GCST005194 | Simple mode
(NOME) | 11 | 0.024187619 | 0.063779583 | 0.712439866; 31 | SLE GWAS | 14 | Coronary
artery disease ∥ id: ebi-a-GCST005194 | Robust adjusted profile score
(RAPS) | 11 | 0.103875724 | 0.043413437 | 0.016724496; 31 | SLE GWAS | 14 | Coronary
artery disease ∥ id: ebi-a-
GCST005194 | Raw | 11 | 0.083799516 | 0.03222313 | 0.026464176; 31 | SLE
GWAS | 14 | Coronary artery disease ∥ id: ebi-a-GCST005194 | Outlier-
corrected | NA | NA | NA | NA; 32 | SLE GWAS | 5 | Coronary artery disease ∥ id: ebi-a-
GCST005194 | Maximum likelihood | 4 | 0.1999604 | 0.12874024 | 0.120373397; 32 | SLE
GWAS | 5 | Coronary artery disease ∥ id: ebi-a-GCST005194 | MR
Egger | 4 | 0.33313951 | 0.19083613 | 0.2229818; 32 | SLE GWAS | 5 | Coronary artery
disease ∥ id: ebi-a-GCST005194 | MR Egger
(bootstrap) | 4 | 0.149147053 | 0.219688216 | 0.149; 32 | SLE GWAS | 5 | Coronary artery
disease ∥ id: ebi-a-GCST005194 | Simple median | 4 | −0.029331485
| 0.294325734 | 0.920617002; 32 | SLE GWAS | 5 | Coronary artery disease ∥
id: ebi-a-GCST005194 | Weighted median | 4 | 0.167476147 | 1.94900449 | 0.931522801;
32 | SLE GWAS | 5 | Coronary artery disease ∥ id: ebi-a-GCST005194 | Penalised weighted
median | 4 | 0.167476147 | 0.474044417 | 0.723869468; 32 | SLE GWAS | 5 | Coronary
artery disease ∥ id: ebi-a-GCST005194 | Inverse variance
weighted | 4 | 0.172274435 | 0.083197597 | 0.038390019; 32 | SLE GWAS | 5 | Coronary
artery disease ∥ id: ebi-a-GCST005194 | Inverse variance weighted (multiplicative random
effects) | 4 | 0.172274435 | 0.053987053 | 0.001417654; 32 | SLE GWAS | 5 | Coronary
artery disease ∥ id: ebi-a-GCST005194 | Inverse variance weighted (fixed
effects) | 4 | 0.172274435 | 0.083197597 | 0.038390019; 32 | SLE GWAS | 5 | Coronary
artery disease ∥ id: ebi-a-GCST005194 | Simple
mode | 4 | 0.105444338 | 0.178816309 | 0.596871403; 32 | SLE GWAS | 5 | Coronary
artery disease ∥ id: ebi-a-GCST005194 | Weighted
mode | 4 | 0.167138891 | 0.121370705 | 0.262247488; 32 | SLE GWAS | 5 | Coronary
artery disease ∥ id: ebi-a-GCST005194 | Weighted mode
(NOME) | 4 | 0.1833743 | 0.090714124 | 0.136464658; 32 | SLE GWAS | 5 | Coronary
artery disease ∥ id: ebi-a-GCST005194 | Simple mode
(NOME) | 4 | 0.105444338 | 0.142995332 | 0.514319259; 32 | SLE GWAS | 5 | Coronary
artery disease ∥ id: ebi-a-GCST005194 | Robust adjusted profile score
(RAPS) | 4 | 0.199954274 | 0.137070538 | 0.144628651; 32 | SLE GWAS | 5 | Coronary
artery disease ∥ id: ebi-a-
GCST005194 | Raw | 4 | 0.172274435 | 0.053987053 | 0.049671764; 32 | SLE
GWAS | 5 | Coronary artery disease ∥ id: ebi-a-GCST005194 | Outlier-
corrected | NA | NA | NA | NA; 33 | SLE GWAS | 51 | Coronary artery disease ∥ id: ebi-a-
GCST005194 | Maximum likelihood | 45 | −0.012390755 | 0.010214949 | 0.225129015;
33 | SLE GWAS | 51 | Coronary artery disease ∥ id: ebi-a-GCST005194 | MR Egger | 45
| −0.018717662 | 0.029022719 | 0.522397868; 33 | SLE GWAS | 51 | Coronary artery disease
∥ id: ebi-a-GCST005194 | MR Egger (bootstrap) | 45 | −0.010437306 | 0.023427125 | 0.311;
33 | SLE GWAS | 51 | Coronary artery disease ∥ id: ebi-a-GCST005194 | Simple
median | 45 | −0.027951057 | 0.016374855 | 0.087831377; 33 | SLE GWAS | 51 | Coronary
artery disease ∥ id: ebi-a-GCST005194 | Weighted median | 45 | −0.028053816
| 0.010789715 | 0.009320977; 33 | SLE GWAS | 51 | Coronary artery disease
∥ id: ebi-a-GCST005194 | Penalised weighted median | 45
| −0.02859096 | 0.010738604 | 0.007757484; 33 | SLE GWAS | 51 | Coronary artery disease ∥
id: ebi-a-GCST005194 | Inverse variance weighted | 45 | −0.01061875
| 0.01830562 | 0.561859704; 33 | SLE GWAS | 51 | Coronary artery disease ∥
id: ebi-a-GCST005194 | Inverse variance weighted (multiplicative random effects) | 45
| −0.01061875 | 0.01830562 | 0.561859704; 33 | SLE GWAS | 51 | Coronary artery disease ∥
id: ebi-a-GCST005194 | Inverse variance weighted (fixed effects) | 45 | −0.01061875
| 0.007701408 | 0.167954442; 33 | SLE GWAS | 51 | Coronary artery disease ∥
id: ebi-a-GCST005194 | Simple mode | 45 | −0.018755589 | 0.021221783 | 0.381613595;
33 | SLE GWAS | 51 | Coronary artery disease ∥ id: ebi-a-GCST005194 | Weighted
mode | 45 | −0.018755589 | 0.015078603 | 0.220139241; 33 | SLE GWAS | 51 | Coronary
artery disease ∥ id: ebi-a-GCST005194 | Weighted mode (NOME) | 45 | −0.018755589
| 0.009891484 | 0.064520791; 33 | SLE GWAS | 51 | Coronary artery disease
∥ id: ebi-a-GCST005194 | Simple mode (NOME) | 45 | −0.018755589
| 0.016897791 | 0.273052848; 33 | SLE GWAS | 51 | Coronary artery disease
∥ id: ebi-a-GCST005194 | Robust adjusted profile score (RAPS) | 45
| −0.016440471 | 0.020374046 | 0.419705627; 33 | SLE GWAS | 51 | Coronary artery disease
∥ id: ebi-a-GCST005194 | Raw | 45 | −0.01061875 | 0.01830562 | 0.564818642; 33 | SLE
GWAS | 51 | Coronary artery disease ∥ id: ebi-a-GCST005194 | Outlier-corrected | 39
| −0.025133733 | 0.012663371 | 0.054425597; 34 | SLE GWAS | 31 | Coronary artery disease
∥ id: ebi-a-GCST005194 | Maximum likelihood | 25
| −0.106540608 | 0.010744459 | 3.55166E−23; 34 | SLE GWAS | 31 | Coronary artery disease
∥ id: ebi-a-GCST005194 | MR Egger | 25 -0.176987492 | 0.020892847 | 1.58464E−08;
34 | SLE GWAS | 31 | Coronary artery disease ∥ id: ebi-a-GCST005194 | MR Egger
(bootstrap) | 25 | 0.133767525 | 0.049585389 | 0.008; 34 | SLE GWAS | 31 | Coronary
artery disease ∥ id: ebi-a-GCST005194 | Simple median | 25 | −0.132893895
| 0.01368908 | 2.78693E−22; 34 | SLE GWAS | 31 | Coronary artery disease ∥
id: ebi-a-GCST005194 | Weighted median | 25 | −0.130119405 | 0.014001992 | 1.50111E−20;
34 | SLE GWAS | 31 | Coronary artery disease ∥ id: ebi-a-GCST005194 | Penalised
weighted median | 25 | −0.133444458 | 0.014199029 | 5.55363E−21; 34 | SLE
GWAS | 31 | Coronary artery disease ∥ id: ebi-a-GCST005194 | Inverse variance
weighted | 25 | −0.094832056 | 0.015361258 | 6.6813E−10; 34 | SLE GWAS | 31 | Coronary
artery disease ∥ id: ebi-a-GCST005194 | Inverse variance weighted (multiplicative random
effects) | 25 | −0.094832056 | 0.015361258 | 6.6813E−10; 34 | SLE GWAS | 31 | Coronary
artery disease ∥ id: ebi-a-GCST005194 | Inverse variance weighted (fixed effects) | 25
| −0.094832056 | 0.008742343 | 2.05086E−27; 34 | SLE GWAS | 31 | Coronary artery disease
∥ id: ebi-a-GCST005194 | Simple mode | 25 | −0.133854863 | 0.019022283 | 2.82701E−07;
34 | SLE GWAS | 31 | Coronary artery disease ∥ id: ebi-a-GCST005194 | Weighted
mode | 25 | −0.133854863 | 0.018182362 | 1.33079E−07; 34 | SLE GWAS | 31 | Coronary
artery disease ∥ id: ebi-a-GCST005194 | Weighted mode (NOME) | 25 | −0.133854863
| 0.014680327 | 2.89013E−09; 34 | SLE GWAS | 31 | Coronary artery disease
∥ id: ebi-a-GCST005194 | Simple mode (NOME) | 25 | −0.133854863
| 0.015252759 | 5.9032E−09; 34 | SLE GWAS | 31 | Coronary artery disease ∥
id: ebi-a-GCST005194 | Robust adjusted profile score (RAPS) | 25 | −0.11727425
| 0.013696681 | 1.10697E−17; 34 | SLE GWAS | 31 | Coronary artery disease ∥
id: ebi-a-GCST005194 | Raw | 25 | −0.094832056 | 0.015361258 | 2.22765E−06; 34 | SLE
GWAS | 31 | Coronary artery disease ∥ id: ebi-a-GCST005194 | Outlier-corrected | 22
| −0.118621756 | 0.008389691 | 3.3451E−12; 35 | SLE GWAS | 19 | Coronary artery disease ∥
id: ebi-a-GCST005194 | Maximum likelihood | 16 | 0.074400943 | 0.011944786 | 4.70207E−10;
35 | SLE GWAS | 19 | Coronary artery disease ∥ id: ebi-a-GCST005194 | MR
Egger | 16 | 0.038262595 | 0.024316018 | 0.13791164; 35 | SLE GWAS | 19 | Coronary
artery disease ∥ id: ebi-a-GCST005194 | MR Egger
(bootstrap) | 16 | 0.055080535 | 0.025919553 | 0.017; 35 | SLE GWAS | 19 | Coronary
artery disease ∥ id: ebi-a-GCST005194 | Simple
median | 16 | 0.070212653 | 0.018397469 | 0.000135397; 35 | SLE GWAS | 19 | Coronary
artery disease ∥ id: ebi-a-GCST005194 | Weighted
median | 16 | 0.066303968 | 0.014921241 | 8.84676E−06; 35 | SLE GWAS | 19 | Coronary
artery disease ∥ id: ebi-a-GCST005194 | Penalised weighted
median | 16 | 0.066303968 | 0.015722848 | 2.47524E−05; 35 | SLE GWAS | 19 | Coronary
artery disease ∥ id: ebi-a-GCST005194 | Inverse variance
weighted | 16 | 0.069901897 | 0.01324241 | 1.30147E−07; 35 | SLE GWAS | 19 | Coronary
artery disease ∥ id: ebi-a-GCST005194 | Inverse variance weighted (multiplicative random
effects) | 16 | 0.069901897 | 0.01324241 | 1.30147E−07; 35 | SLE GWAS | 19 | Coronary
artery disease ∥ id: ebi-a-GCST005194 | Inverse variance weighted (fixed
effects) | 16 | 0.069901897 | 0.010853864 | 1.19255E−10; 35 | SLE GWAS | 19 | Coronary
artery disease ∥ id: ebi-a-GCST005194 | Simple
mode | 16 | 0.056480813 | 0.02015768 | 0.013406871; 35 | SLE GWAS | 19 | Coronary
artery disease ∥ id: ebi-a-GCST005194 | Weighted
mode | 16 | 0.059302565 | 0.015335238 | 0.001519985; 35 | SLE GWAS | 19 | Coronary
artery disease ∥ id: ebi-a-GCST005194 | Weighted mode
(NOME) | 16 | 0.060713441 | 0.014221383 | 0.00067226; 35 | SLE GWAS | 19 | Coronary
artery disease ∥ id: ebi-a-GCST005194 | Simple mode
(NOME) | 16 | 0.056480813 | 0.019415508 | 0.010794555; 35 | SLE GWAS | 19 | Coronary
artery disease ∥ id: ebi-a-GCST005194 | Robust adjusted profile score
(RAPS) | 16 | 0.068377 | 0.014778282 | 3.71256E−06; 35 | SLE GWAS | 19 | Coronary
artery disease ∥ id: ebi-a-GCST005194 | Raw | 16 | 0.069901897 | 0.01324241 | 9.27408E−05;
35 | SLE GWAS | 19 | Coronary artery disease ∥ id: ebi-a-GCST005194 | Outlier-
corrected | NA | NA | NA | NA; 36 | SLE GWAS | 2 | Coronary artery disease ∥ id: ebi-a-
GCST005194 | Maximum likelihood | 2 | −0.405317764 | 0.832295512 | 0.626266973;
36 | SLE GWAS | 2 | Coronary artery disease ∥ id: ebi-a-GCST005194 | Inverse variance
weighted | 2 | −0.229730844 | 0.239799972 | 0.338057562; 36 | SLE GWAS | 2 | Coronary
artery disease ∥ id: ebi-a-GCST005194 | Inverse variance weighted (multiplicative random
effects) | 2 | −0.229730844 | 0.239799972 | 0.338057562; 36 | SLE GWAS | 2 | Coronary
artery disease ∥ id: ebi-a-GCST005194 | Inverse variance weighted (fixed effects) | 2
| −0.229730844 | 0.235932639 | 0.330198709; 36 | SLE GWAS | 2 | Coronary artery disease ∥
id: ebi-a-GCST005194 | Robust adjusted profile score (RAPS) | 2 | −0.405321801
| 0.983209326 | 0.680160847; 37 | SLE GWAS | 9 | Coronary artery disease ∥
id: ebi-a-GCST005194 | Maximum
likelihood | 7 | 0.072291822 | 0.063505167 | 0.254969666; 37 | SLE GWAS | 9 | Coronary
artery disease ∥ id: ebi-a-GCST005194 | MR Egger | 7 | −0.126353312
| 0.128819964 | 0.371711063; 37 | SLE GWAS | 9 | Coronary artery disease ∥
id: ebi-a-GCST005194 | MR Egger (bootstrap) | 7 | −0.016492138 | 0.170040871 | 0.457;
37 | SLE GWAS | 9 | Coronary artery disease ∥ id: ebi-a-GCST005194 | Simple
median | 7 | 0.04840523 | 0.080364625 | 0.546961076; 37 | SLE GWAS | 9 | Coronary
artery disease ∥ id: ebi-a-GCST005194 | Weighted
median | 7 | 0.000251349 | 0.051013285 | 0.99606874; 37 | SLE GWAS | 9 | Coronary
artery disease ∥ id: ebi-a-GCST005194 | Penalised weighted median | 7
| −0.000120648 | 0.054912048 | 0.998246954; 37 | SLE GWAS | 9 | Coronary artery disease ∥
id: ebi-a-GCST005194 | Inverse variance
weighted | 7 | 0.025379979 | 0.074684037 | 0.733983514; 37 | SLE GWAS | 9 | Coronary
artery disease ∥ id: ebi-a-GCST005194 | Inverse variance weighted (multiplicative random
effects) | 7 | 0.025379979 | 0.074684037 | 0.733983514; 37 | SLE GWAS | 9 | Coronary
artery disease ∥ id: ebi-a-GCST005194 | Inverse variance weighted (fixed
effects) | 7 | 0.025379979 | 0.032394841 | 0.433358538; 37 | SLE GWAS | 9 | Coronary
artery disease ∥ id: ebi-a-GCST005194 | Simple mode | 7 | −0.02330335
| 0.060710398 | 0.714327518; 37 | SLE GWAS | 9 | Coronary artery disease ∥
id: ebi-a-GCST005194 | Weighted mode | 7 | −0.012678267 | 0.043426292 | 0.780157054;
37 | SLE GWAS | 9 | Coronary artery disease ∥ id: ebi-a-GCST005194 | Weighted mode
(NOME) | 7 | −0.02330335 | 0.042408716 | 0.602502332; 37 | SLE GWAS | 9 | Coronary
artery disease ∥ id: ebi-a-GCST005194 | Simple mode (NOME) | 7 | −0.02330335
| 0.058146083 | 0.702469052; 37 | SLE GWAS | 9 | Coronary artery disease ∥
id: ebi-a-GCST005194 | Robust adjusted profile score (RAPS) | 7 | −0.000272286
| 0.08432586 | 0.997423658; 37 | SLE GWAS | 9 | Coronary artery disease ∥
id: ebi-a-GCST005194 | Raw | 7 | 0.025379979 | 0.074684037 | 0.745564068; 37 | SLE
GWAS | 9 | Coronary artery disease ∥ id: ebi-a-GCST005194 | Outlier-
corrected | 5 | 0.028063473 | 0.051248083 | 0.613087553; 38 | SLE GWAS | 10 | Coronary
artery disease ∥ id: ebi-a-GCST005194 | Maximum likelihood | 10 | −0.003105316
| 0.033275752 | 0.925648792; 38 | SLE GWAS | 10 | Coronary artery disease
∥ id: ebi-a-GCST005194 | MR Egger | 10 | −0.093195349 | 0.101134492 | 0.383737628;
38 | SLE GWAS | 10 | Coronary artery disease ∥ id: ebi-a-GCST005194 | MR Egger
(bootstrap) | 10 | −0.037514691 | 0.1070968 | 0.364; 38 | SLE GWAS | 10 | Coronary artery
disease ∥ id: ebi-a-GCST005194 | Simple
median | 10 | 0.018801465 | 0.045060426 | 0.676495515; 38 | SLE GWAS | 10 | Coronary
artery disease ∥ id: ebi-a-GCST005194 | Weighted median | 10 | −0.023490087
| 0.036464661 | 0.519453706; 38 | SLE GWAS | 10 | Coronary artery disease
∥ id: ebi-a-GCST005194 | Penalised weighted median | 10 | −0.023525201
| 0.038176459 | 0.537747548; 38 | SLE GWAS | 10 | Coronary artery disease
∥ id: ebi-a-GCST005194 | Inverse variance weighted | 10 | −0.001991513
| 0.048284789 | 0.967100464; 38 | SLE GWAS | 10 | Coronary artery disease
∥ id: ebi-a-GCST005194 | Inverse variance weighted (multiplicative random effects) | 10
| −0.001991513 | 0.048284789 | 0.967100464; 38 | SLE GWAS | 10 | Coronary artery disease
∥ id: ebi-a-GCST005194 | Inverse variance weighted (fixed effects) | 10 | −0.001991513
| 0.024463338 | 0.935117431; 38 | SLE GWAS | 10 | Coronary artery disease
∥ id: ebi-a-GCST005194 | Simple mode | 10 | 0.012077367 | 0.052740192 | 0.823989931;
38 | SLE GWAS | 10 | Coronary artery disease ∥ id: ebi-a-GCST005194 | Weighted
mode | 10 | −0.018864272 | 0.037696523 | 0.628783916; 38 | SLE GWAS | 10 | Coronary
artery disease ∥ id: ebi-a-GCST005194 | Weighted mode (NOME) | 10 | −0.018864272
| 0.037375905 | 0.625883005; 38 | SLE GWAS | 10 | Coronary artery disease
∥ id: ebi-a-GCST005194 | Simple mode
(NOME) | 10 | 0.012077367 | 0.050937406 | 0.817886518; 38 | SLE GWAS | 10 | Coronary
artery disease ∥ id: ebi-a-GCST005194 | Robust adjusted profile score (RAPS) | 10
| −0.02116154 | 0.044395829 | 0.633607068; 38 | SLE GWAS | 10 | Coronary artery disease ∥
id: ebi-a-GCST005194 | Raw | 10 | −0.001991513 | 0.048284789 | 0.968000968; 38 | SLE
GWAS | 10 | Coronary artery disease ∥ id: ebi-a-GCST005194 | Outlier-corrected | 8
| −0.003148674 | 0.029342249 | 0.917555045; 39 | SLE GWAS | 7 | Coronary artery disease
id: ebi-a-GCST005194 | Maximum likelihood | 7 | −0.023919495
| 0.028785669 | 0.406001014; 39 | SLE GWAS | 7 | Coronary artery disease ∥
id: ebi-a-GCST005194 | MR Egger | 7 | 0.230059194 | 0.118993771 | 0.11100958; 39 | SLE
GWAS | 7 | Coronary artery disease ∥ id: ebi-a-GCST005194 | MR Egger
(bootstrap) | 7 | 0.074217801 | 0.070925375 | 0.122; 39 | SLE GWAS | 7 | Coronary artery
disease ∥ id: ebi-a-GCST005194 | Simple median | 7
| −0.069623583 | 0.046572797 | 0.134929845; 39 | SLE GWAS | 7 | Coronary artery disease ∥
id: ebi-a-GCST005194 | Weighted median | 7 | 0.014454787 | 0.035553677 | 0.684329436;
39 | SLE GWAS | 7 | Coronary artery disease ∥ id: ebi-a-GCST005194 | Penalised weighted
median | 7 | 0.014454787 | 0.036465696 | 0.691814307; 39 | SLE GWAS | 7 | Coronary
artery disease ∥ id: ebi-a-GCST005194 | Inverse variance weighted | 7 | −0.020541443
| 0.030679014 | 0.503138274; 39 | SLE GWAS | 7 | Coronary artery disease ∥
id: ebi-a-GCST005194 | Inverse variance weighted (multiplicative random effects) | 7
| −0.020541443 | 0.030679014 | 0.503138274; 39 | SLE GWAS | 7 | Coronary artery disease ∥
id: ebi-a-GCST005194 | Inverse variance weighted (fixed effects) | 7 | −0.020541443
| 0.026034374 | 0.430104793; 39 | SLE GWAS | 7 | Coronary artery disease ∥
id: ebi-a-GCST005194 | Simple mode | 7 | 0.004908554 | 0.049680403 | 0.924513246;
39 | SLE GWAS | 7 | Coronary artery disease ∥ id: ebi-a-GCST005194 | Weighted
mode | 7 | 0.020714248 | 0.03652858 | 0.591234331; 39 | SLE GWAS | 7 | Coronary artery
disease ∥ id: ebi-a-GCST005194 | Weighted mode
(NOME) | 7 | 0.020714248 | 0.036035847 | 0.586302368; 39 | SLE GWAS | 7 | Coronary
artery disease ∥ id: ebi-a-GCST005194 | Simple mode
(NOME) | 7 | 0.004908554 | 0.053637337 | 0.930063237; 39 | SLE GWAS | 7 | Coronary
artery disease ∥ id: ebi-a-GCST005194 | Robust adjusted profile score (RAPS) | 7
| −0.026698495 | 0.037729497 | 0.479175541; 39 | SLE GWAS | 7 | Coronary artery disease ∥
id: ebi-a-GCST005194 | Raw | 7 | −0.020541443 | 0.030679014 | 0.528048624; 39 | SLE
GWAS | 7 | Coronary artery disease ∥ id: ebi-a-GCST005194 | Outlier-
corrected | NA | NA | NA | NA; 40 | SLE GWAS | 37 | Coronary artery disease ∥ id: ebi-a-
GCST005194 | Maximum likelihood | 33 | −0.065792965 | 0.006310014 | 1.87172E−25;
40 | SLE GWAS | 37 | Coronary artery disease ∥ id: ebi-a-GCST005194 | MR Egger | 33
| −0.07695487 | 0.02672363 | 0.007156897; 40 | SLE GWAS | 37 | Coronary artery disease ∥
id: ebi-a-GCST005194 | MR Egger (bootstrap) | 33 | −0.040987689 | 0.025175017 | 0.059;
40 | SLE GWAS | 37 | Coronary artery disease ∥ id: ebi-a-GCST005194 | Simple
median | 33 | −0.06453245 | 0.00817179 | 2.85746E−15; 40 | SLE GWAS | 37 | Coronary
artery disease ∥ id: ebi-a-GCST005194 | Weighted median | 33 | −0.064254009
| 0.007872311 | 3.2945E−16; 40 | SLE GWAS | 37 | Coronary artery disease
id: ebi-a-GCST005194 | Penalised weighted median | 33 | −0.064329866
| 0.00830242 | 9.31108E−15; 40 | SLE GWAS | 37 | Coronary artery disease ∥
id: ebi-a-GCST005194 | Inverse variance weighted | 33 | −0.063284571
| 0.005836843 | 2.17043E−27; 40 | SLE GWAS | 37 | Coronary artery disease
∥ id: ebi-a-GCST005194 | Inverse variance weighted (multiplicative random effects) | 33
| −0.063284571 | 0.004566044 | 1.10944E−43; 40 | SLE GWAS | 37 | Coronary artery disease
∥ id: ebi-a-GCST005194 | Inverse variance weighted (fixed effects) | 33 | −0.063284571
| 0.005836843 | 2.17043E−27; 40 | SLE GWAS | 37 | Coronary artery disease
∥ id: ebi-a-GCST005194 | Simple mode | 33 | −0.07037917 | 0.014904432 | 4.44995E−05;
40 | SLE GWAS | 37 | Coronary artery disease ∥ id: ebi-a-GCST005194 | Weighted
mode | 33 | −0.07037917 | 0.014276144 | 2.4364E−05; 40 | SLE GWAS | 37 | Coronary
artery disease ∥ id: ebi-a-GCST005194 | Weighted mode (NOME) | 33 | −0.07037917
| 0.013210942 | 7.6654E−06; 40 | SLE GWAS | 37 | Coronary artery disease ∥
id: ebi-a-GCST005194 | Simple mode (NOME) | 33 | −0.07037917
| 0.013592412 | 1.18464E−05; 40 | SLE GWAS | 37 | Coronary artery disease ∥
id: ebi-a-GCST005194 | Robust adjusted profile score (RAPS) | 33 | −0.065339501
| 0.006414506 | 2.28503E−24; 40 | SLE GWAS | 37 | Coronary artery disease
∥ id: ebi-a-GCST005194 | Raw | 33 | −0.063284571 | 0.004566044 | 4.49593E−15; 40 | SLE
GWAS | 37 | Coronary artery disease ∥ id: ebi-a-GCST005194 | Outlier-
corrected | NA | NA | NA | NA; 41 | SLE GWAS | 5 | Coronary artery disease ∥ id: ebi-a-
GCST005194 | Maximum likelihood | 4 | 0.144602098 | 0.058378924 | 0.013250778;
41 | SLE GWAS | 5 | Coronary artery disease ∥ id: ebi-a-GCST005194 | MR
Egger | 4 | 0.021843947 | 0.136486073 | 0.887548724; 41 | SLE GWAS | 5 | Coronary
artery disease ∥ id: ebi-a-GCST005194 | MR Egger
(bootstrap) | 4 | 0.087099554 | 0.123381251 | 0.195; 41 | SLE GWAS | 5 | Coronary artery
disease ∥ id: ebi-a-GCST005194 | Simple
median | 4 | 0.117931162 | 0.127060811 | 0.353331113; 41 | SLE GWAS | 5 | Coronary
artery disease ∥ id: ebi-a-GCST005194 | Weighted
median | 4 | 0.09835273 | 0.065910855 | 0.135644524; 41 | SLE GWAS | 5 | Coronary
artery disease ∥ id: ebi-a-GCST005194 | Penalised weighted
median 4 0.09835273 | 0.081711957 | 0.228724255; 41 | SLE GWAS | 5 | Coronary
artery disease ∥ id: ebi-a-GCST005194 | Inverse variance
weighted | 4 | 0.122985753 | 0.047303564 | 0.009324394; 41 | SLE GWAS | 5 | Coronary
artery disease ∥ id: ebi-a-GCST005194 | Inverse variance weighted (multiplicative random
effects) | 4 | 0.122985753 | 0.047303564 | 0.009324394; 41 | SLE GWAS | 5 | Coronary
artery disease ∥ id: ebi-a-GCST005194 | Inverse variance weighted (fixed
effects) | 4 | 0.122985753 | 0.042556667 | 0.003853286; 41 | SLE GWAS | 5 | Coronary
artery disease ∥ id: ebi-a-GCST005194 | Simple
mode | 4 | 0.058152119 | 0.098228987 | 0.5954994; 41 | SLE GWAS | 5 | Coronary artery
disease ∥ id: ebi-a-GCST005194 | Weighted
mode | 4 | 0.056875544 | 0.059906048 | 0.412453407; 41 | SLE GWAS | 5 | Coronary
artery disease ∥ id: ebi-a-GCST005194 | Weighted mode
(NOME) | 4 | 0.07219445 | 0.052205601 | 0.260654106; 41 | SLE GWAS | 5 | Coronary
artery disease ∥ id: ebi-a-GCST005194 | Simple mode
(NOME) | 4 | 0.058152119 | 0.083160317 | 0.534719685; 41 | SLE GWAS | 5 | Coronary
artery disease ∥ id: ebi-a-GCST005194 | Robust adjusted profile score
(RAPS) | 4 | 0.144614591 | 0.059661415 | 0.015353921; 41 | SLE GWAS | 5 | Coronary
artery disease ∥ id: ebi-a-
GCST005194 | Raw | 4 | 0.122985753 | 0.047303564 | 0.080380981; 41 | SLE
GWAS | 5 | Coronary artery disease ∥ id: ebi-a-GCST005194 | Outlier-
corrected | NA | NA | NA | NA; 42 | SLE GWAS | 79 | Coronary artery disease ∥ id: ebi-a-
GCST005194 | Maximum likelihood 65 | 0.023997617 | 0.006786943 | 0.000406465;
42 | SLE GWAS | 79 | Coronary artery disease ∥ id: ebi-a-GCST005194 | MR
Egger | 65 | 0.0179139 | 0.023089884 | 0.440750241; 42 | SLE GWAS | 79 | Coronary
artery disease ∥ id: ebi-a-GCST005194 | MR Egger
(bootstrap) | 65 | 0.010505166 | 0.014065276 | 0.229; 42 | SLE GWAS | 79 | Coronary
artery disease ∥ id: ebi-a-GCST005194 | Simple
median | 65 | 0.026473587 | 0.010857467 | 0.014757182; 42 | SLE GWAS | 79 | Coronary
artery disease ∥ id: ebi-a-GCST005194 | Weighted
median | 65 | 0.022365344 | 0.008839541 | 0.011401453; 42 | SLE GWAS | 79 | Coronary
artery disease ∥ id: ebi-a-GCST005194 | Penalised weighted
median | 65 | 0.021314128 | 0.009103081 | 0.01921059; 42 | SLE GWAS | 79 | Coronary
artery disease ∥ id: ebi-a-GCST005194 | Inverse variance
weighted | 65 | 0.0212703 | 0.009582954 | 0.026446106; 42 | SLE GWAS | 79 | Coronary
artery disease ∥ id: ebi-a-GCST005194 | Inverse variance weighted (multiplicative random
effects) | 65 | 0.0212703 | 0.009582954 | 0.026446106; 42 | SLE GWAS | 79 | Coronary
artery disease ∥ id: ebi-a-GCST005194 | Inverse variance weighted (fixed
effects) | 65 | 0.0212703 | 0.0062538 | 0.000670956; 42 | SLE GWAS | 79 | Coronary artery
disease ∥ id: ebi-a-GCST005194 | Simple
mode | 65 | 0.02168865 | 0.011916899 | 0.07343716; 42 | SLE GWAS | 79 | Coronary
artery disease ∥ id: ebi-a-GCST005194 | Weighted
mode | 65 | 0.016033235 | 0.010430289 | 0.129178743; 42 | SLE GWAS | 79 | Coronary
artery disease ∥ id: ebi-a-GCST005194 | Weighted mode
(NOME) | 65 0.016033235 | 0.010420714 | 0.1288336; 42 | SLE GWAS | 79 | Coronary
artery disease ∥ id: ebi-a-GCST005194 | Simple mode
(NOME) | 65 | 0.02168865 | 0.011757263 | 0.069709222; 42 | SLE GWAS | 79 | Coronary
artery disease ∥ id: ebi-a-GCST005194 | Robust adjusted profile score
(RAPS) | 65 | 0.020611509 | 0.009540527 | 0.030740465; 42 | SLE GWAS | 79 | Coronary
artery disease ∥ id: ebi-a-
GCST005194 | Raw | 65 | 0.0212703 | 0.009582954 | 0.029996569; 42 | SLE
GWAS | 79 | Coronary artery disease ∥ id: ebi-a-GCST005194 | Outlier-
corrected | 62 | 0.021649501 | 0.007993826 | 0.008764323; 43 | SLE GWAS | 8 | Coronary
artery disease ∥ id: ebi-a-GCST005194 | Maximum likelihood | 8 | 7.99752E−05 |
0.021227325 | 0.996993929; 43 | SLE GWAS | 8 | Coronary artery disease ∥ id: ebi-a-
GCST005194 | MR Egger | 8 | −0.028445191 | 0.045408664 | 0.554104607; 43 | SLE
GWAS | 8 | Coronary artery disease ∥ id: ebi-a-GCST005194 | MR Egger (bootstrap) | 8
| −0.014848258 | 0.093128245 | 0.437; 43 | SLE GWAS | 8 | Coronary artery disease ∥ id: ebi-
a-GCST005194 | Simple median | 8 | 0.015208247 | 0.028835759 | 0.597909407; 43 | SLE
GWAS | 8 | Coronary artery disease ∥ id: ebi-a-GCST005194 | Weighted
median | 8 | 0.009856703 | 0.025035761 | 0.693798885; 43 | SLE GWAS | 8 | Coronary
artery disease ∥ id: ebi-a-GCST005194 | Penalised weighted
median | 8 | 0.010040404 | 0.025379493 | 0.692392082; 43 | SLE GWAS | 8 | Coronary
artery disease ∥ id: ebi-a-GCST005194 | Inverse variance weighted | 8 | 7.18604E−05
| 0.024489339 | 0.997658728; 43 | SLE GWAS | 8 | Coronary artery disease ∥ id: ebi-a-
GCST005194 | Inverse variance weighted (multiplicative random effects) | 8 | 7.18604E−05
| 0.024489339 | 0.997658728; 43 | SLE GWAS | 8 | Coronary artery disease ∥ id: ebi-a-
GCST005194 | Inverse variance weighted (fixed effects) | 8 | 7.18604E−05
| 0.019992381 | 0.997132099; 43 | SLE GWAS | 8 | Coronary artery disease ∥ id: ebi-a-
GCST005194 | Simple mode | 8 | 0.012228051 | 0.030099771 | 0.696690958; 43 | SLE
GWAS | 8 | Coronary artery disease ∥ id: ebi-a-GCST005194 | Weighted
mode | 8 | 0.008953687 | 0.027229092 | 0.751904557; 43 | SLE GWAS | 8 | Coronary
artery disease ∥ id: ebi-a-GCST005194 | Weighted mode
(NOME) | 8 | 0.008953687 | 0.026895098 | 0.748949559; 43 | SLE GWAS | 8 | Coronary
artery disease ∥ id: ebi-a-GCST005194 | Simple mode
(NOME) | 8 | 0.012228051 | 0.030179482 | 0.697443803; 43 | SLE GWAS | 8 | Coronary
artery disease ∥ id: ebi-a-GCST005194 | Robust adjusted profile score
(RAPS) | 8 | 0.008695108 | 0.023440709 | 0.710681868; 43 | SLE GWAS | 8 | Coronary
artery disease ∥ id: ebi-a-GCST005194 | Raw | 8 | 7.18604E−05
| 0.024489339 | 0.997740601; 43 | SLE GWAS | 8 | Coronary artery disease ∥ id: ebi-a-
GCST005194 | Outlier-corrected | NA | NA | NA | NA; 44 | SLE GWAS | 21 | Coronary
artery disease ∥ id: ebi-a-GCST005194 | Maximum
likelihood 17 | 0.044250374 | 0.011553742 | 0.000128164; 44 | SLE
GWAS | 21 | Coronary artery disease ∥ id: ebi-a-GCST005194 | MR
Egger | 17 | 0.048938181 | 0.021128157 | 0.035102917; 44 | SLE GWAS | 21 | Coronary
artery disease ∥ id: ebi-a-GCST005194 | MR Egger
(bootstrap) | 17 | 0.043607794 | 0.029867846 | 0.067; 44 | SLE GWAS | 21 | Coronary
artery disease ∥ id: ebi-a-GCST005194 | Simple
median | 17 | 0.045710485 | 0.015428727 | 0.003049668; 44 | SLE GWAS | 21 | Coronary
artery disease ∥ id: ebi-a-GCST005194 | Weighted
median | 17 | 0.045710485 | 0.01414023 | 0.001226456; 44 | SLE GWAS | 21 | Coronary
artery disease ∥ id: ebi-a-GCST005194 | Penalised weighted
median | 17 | 0.045710485 | 0.014600626 | 0.001743778; 44 | SLE GWAS | 21 | Coronary
artery disease ∥ id: ebi-a-GCST005194 | Inverse variance
weighted | 17 | 0.044359369 | 0.011298657 | 8.63435E−05; 44 | SLE GWAS | 21 | Coronary
artery disease ∥ id: ebi-a-GCST005194 | Inverse variance weighted (multiplicative random
effects) | 17 | 0.044359369 | 0.004105314 | 3.247E−27; 44 | SLE GWAS | 21 | Coronary
artery disease ∥ id: ebi-a-GCST005194 | Inverse variance weighted (fixed
effects) | 17 | 0.044359369 | 0.011298657 | 8.63435E−05; 44 | SLE GWAS | 21 | Coronary
artery disease ∥ id: ebi-a-GCST005194 | Simple
mode | 17 | 0.045839232 | 0.017452763 | 0.018327118; 44 | SLE GWAS | 21 | Coronary
artery disease ∥ id: ebi-a-GCST005194 | Weighted
mode | 17 | 0.045839232 | 0.016065544 | 0.011503018; 44 | SLE GWAS | 21 | Coronary
artery disease ∥ id: ebi-a-GCST005194 | Weighted mode
(NOME) | 17 | 0.045839232 | 0.01638131 | 0.01288743; 44 | SLE GWAS | 21 | Coronary
artery disease ∥ id: ebi-a-GCST005194 | Simple mode
(NOME) | 17 | 0.045839232 | 0.016639458 | 0.014092792; 44 | SLE GWAS | 21 | Coronary
artery disease ∥ id: ebi-a-GCST005194 | Robust adjusted profile score
(RAPS) | 17 | 0.044622523 | 0.011987122 | 0.00019723; 44 | SLE GWAS | 21 | Coronary
artery disease ∥ id: ebi-a-GCST005194 | Raw | 17 | 0.044359369 | 0.004105314 | 9.2527E−09;
44 | SLE GWAS | 21 | Coronary artery disease ∥ id: ebi-a-GCST005194 | Outlier-
corrected | NA | NA | NA | NA; 45 | SLE GWAS | 43 | Coronary artery disease ∥ id: ebi-a-
GCST005194 | Maximum likelihood | 40 | −0.037772646 | 0.006126983 | 7.0498E−10;
45 | SLE GWAS | 43 | Coronary artery disease ∥ id: ebi-a-GCST005194 | MR Egger | 40
| −0.032941547 | 0.048006536 | 0.496759839; 45 | SLE GWAS | 43 | Coronary artery disease
∥ id: ebi-a-GCST005194 | MR Egger (bootstrap) | 40 | −0.115968904 | 0.029347039 | 0;
45 | SLE GWAS | 43 | Coronary artery disease ∥ id: ebi-a-GCST005194 | Simple
median | 40 | −0.063425914 | 0.008056898 | 3.4832E−15; 45 | SLE GWAS | 43 | Coronary
artery disease ∥ id: ebi-a-GCST005194 | Weighted median | 40 | −0.064074583
| 0.00785538 | 3.44082E−16; 45 | SLE GWAS | 43 | Coronary artery disease ∥
id: ebi-a-GCST005194 | Penalised weighted median | 40 | −0.06411991
| 0.00813668 | 3.26457E−15; 45 | SLE GWAS | 43 | Coronary artery disease ∥
id: ebi-a-GCST005194 | Inverse variance weighted | 40 | −0.034328045
| 0.012056477 | 0.004409594; 45 | SLE GWAS | 43 | Coronary artery disease
∥ id: ebi-a-GCST005194 | Inverse variance weighted (multiplicative random effects) | 40
| −0.034328045 | 0.012056477 | 0.004409594; 45 | SLE GWAS | 43 | Coronary artery disease
∥ id: ebi-a-GCST005194 | Inverse variance weighted (fixed effects) | 40
| −0.034328045 | 0.005406871 | 2.16766E−10; 45 | SLE GWAS | 43 | Coronary artery disease
∥ id: ebi-a-GCST005194 | Simple mode | 40 | −0.070293992 | 0.011822515 | 6.15987E−07;
45 | SLE GWAS | 43 | Coronary artery disease ∥ id: ebi-a-GCST005194 | Weighted
mode | 40 | −0.070293992 | 0.011158123 | 1.98595E−07; 45 | SLE GWAS | 43 | Coronary
artery disease ∥ id: ebi-a-GCST005194 | Weighted mode (NOME) | 40 | −0.071124613
| 0.010081687 | 1.80228E−08; 45 | SLE GWAS | 43 | Coronary artery disease
∥ id: ebi-a-GCST005194 | Simple mode (NOME) | 40 | −0.070293992
| 0.011348876 | 2.78558E−07; 45 | SLE GWAS | 43 | Coronary artery disease
∥ id: ebi-a-GCST005194 | Robust adjusted profile score (RAPS) | 40 | −0.057835532
| 0.006154517 | 5.60081E−21; 45 | SLE GWAS | 43 | Coronary artery disease
∥ id: ebi-a-GCST005194 | Raw | 40 | −0.034328045 | 0.012056477 | 0.006999244; 45 | SLE
GWAS | 43 | Coronary artery disease ∥ id: ebi-a-GCST005194 | Outlier-corrected | 35
| −0.059539145 | 0.005467296 | 1.25716E−12; 46 | SLE GWAS | 73 | Coronary artery disease
∥ id: ebi-a-GCST005194 | Maximum likelihood | 63 | −0.051043543
| 0.006004835 | 1.88927E−17; 46 | SLE GWAS | 73 | Coronary artery disease
∥ id: ebi-a-GCST005194 | MR Egger | 63 | −0.09727318 | 0.015890084 | 7.29295E−08;
46 | SLE GWAS | 73 | Coronary artery disease ∥ id: ebi-a-GCST005194 | MR Egger
(bootstrap) | 63 | −0.091245031 | 0.01864619 | 0.00E+00; 46 | SLE GWAS | 73 | Coronary
artery disease ∥ id: ebi-a-GCST005194 | Simple median | 63 | −0.035072837
| 0.009726908 | 0.000311248; 46 | SLE GWAS | 73 | Coronary artery disease
∥ id: ebi-a-GCST005194 | Weighted median | 63 | −0.063588047 | 0.007666127 | 1.08881E−16;
46 | SLE GWAS | 73 | Coronary artery disease ∥ id: ebi-a-GCST005194 | Penalised
weighted median | 63 | −0.064075669 | 0.008449449 | 3.36578E−14; 46 | SLE
GWAS | 73 | Coronary artery disease ∥ id: ebi-a-GCST005194 | Inverse variance
weighted | 63 | −0.043765551 | 0.008894917 | 8.6417E−07; 46 | SLE GWAS | 73 | Coronary
artery disease ∥ id: ebi-a-GCST005194 | Inverse variance weighted (multiplicative random
effects) | 63 | −0.043765551 | 0.008894917 | 8.6417E−07; 46 | SLE GWAS | 73 | Coronary
artery disease ∥ id: ebi-a-GCST005194 | Inverse variance weighted (fixed effects) | 63
| −0.043765551 | 0.0051911 | 3.43072E−17; 46 | SLE GWAS | 73 | Coronary artery disease ∥
id: ebi-a-GCST005194 | Simple mode | 63 | −0.067871824 | 0.012744861 | 1.48152E−06;
46 | SLE GWAS | 73 | Coronary artery disease ∥ id: ebi-a-GCST005194 | Weighted
mode | 63 | −0.067871824 | 0.011759405 | 2.69714E−07; 46 | SLE GWAS | 73 | Coronary
artery disease ∥ id: ebi-a-GCST005194 | Weighted mode (NOME) | 63 | −0.067871824
| 0.008737052 | 9.99062E−11; 46 | SLE GWAS | 73 | Coronary artery disease
∥ id: ebi-a-GCST005194 | Simple mode (NOME) | 63 | −0.067871824
| 0.010244024 | 9.52371E−09; 46 | SLE GWAS | 73 | Coronary artery disease
∥ id: ebi-a-GCST005194 | Robust adjusted profile score (RAPS) | 63 | −0.047007976
| 0.008797918 | 9.13806E−08; 46 | SLE GWAS | 73 | Coronary artery disease
∥ id: ebi-a-GCST005194 | Raw | 63 | −0.043765551 | 0.008894917 | 6.69581E−06; 46 | SLE
GWAS | 73 | Coronary artery disease ∥ id: ebi-a-GCST005194 | Outlier-corrected | 62
| −0.04576325 | 0.007603892 | 1.08847E−07;

TABLE 3F
67 PPI-based cluser | MR analysis (FIG. 11). Listed by:
Cluster | Exposure_data number of SNPs linking to
each cluster | Outcome | MR method | # SNPs used as IVs | beta SE | pval.

1 | SLE GWAS | 133 | Coronary artery disease || id: ebi-a-GCST005194 | Maximum
likelihood | 115 | −0.021694669 | 0.003899657 | 2.64833E−08; 1 | SLE
GWAS | 133 | Coronary artery disease || id: ebi-a-GCST005194 | MR Egger | 115
| −0.028104951 | 0.013494365 | 0.039533492; 1 | SLE GWAS | 133 | Coronary artery disease
|| id: ebi-a-GCST005194 | MR Egger | (bootstrap) | 115 | 0.009943603 | 0.008463107 | 0.133;
1 | SLE GWAS | 133 | Coronary artery disease || id: ebi-a-GCST005194 | Simple
median | 115 | −0.024687438 | 0.009104503 | 0.006696673; 1 | SLE
GWAS | 133 | Coronary artery disease || id: ebi-a-GCST005194 | Weighted
median | 115 | 0.007414983 | 0.006986954 | 0.288571268; 1 | SLE GWAS | 133 | Coronary
artery disease || id: ebi-a-GCST005194 | Penalised weighted
median | 115 | 0.0218566 | 0.00632451 | 0.000548547; 1 | SLE GWAS | 133 | Coronary
artery disease || id: ebi-a-GCST005194 | Inverse variance weighted | 115
| −0.020597271 | 0.006362712 | 0.001207154; 1 | SLE GWAS | 133 | Coronary artery disease
|| id: ebi-a-GCST005194 | Inverse variance weighted (multiplicative random
effects) | 115 | −0.020597271 | 0.006362712 | 0.001207154; 1 | SLE
GWAS | 133 | Coronary artery disease || id: ebi-a-GCST005194 | Inverse variance weighted
(fixed effects) | 115 | −0.020597271 | 0.003607266 | 1.13016E−08; 1 | SLE
GWAS | 133 | Coronary artery disease || id: ebi-a-GCST005194 | Simple
mode | 115 | 0.017843263 | 0.023811537 | 0.455187584; 1 | SLE GWAS | 133 | Coronary
artery disease || id: ebi-a-GCST005194 | Weighted
mode | 115 | 0.023746616 | 0.013148423 | 0.073550179; 1 | SLE GWAS | 133 | Coronary
artery disease || id: ebi-a-GCST005194 | Weighted mode
(NOME) | 115 | 0.023746616 | 0.009864902 | 0.017681482; 1 | SLE
GWAS | 133 | Coronary artery disease || id: ebi-a-GCST005194 | Simple mode
(NOME) | 115 | 0.017843263 | 0.03004369 | 0.55374866; 1 | SLE GWAS | 133 | Coronary
artery disease || id: ebi-a-GCST005194 | Robust adjusted profile score (RAPS) | 115
| −0.03068692 | 0.007200353 | 2.0273E−05; 1 | SLE GWAS | 133 | Coronary artery disease ||
id: ebi-a-GCST005194 | Raw | 115 | −0.020597271 | 0.006362712 | 0.001580969; 1 | SLE
GWAS | 133 | Coronary artery disease || id: ebi-a-GCST005194 | Outlier-corrected | 113
| −0.019367366 | 0.006187551 | 0.002229074; 2 | SLE GWAS | 191 | Coronary artery disease
|| id: ebi-a-GCST005194 | Maximum
likelihood | 168 | 0.016670264 | 0.002492865 | 2.27495E−11; 2 | SLE
GWAS | 191 | Coronary artery disease || id: ebi-a-GCST005194 | MR
Egger | 168 | 0.014540062 | 0.005179966 | 0.005599732; 2 | SLE GWAS | 191 | Coronary
artery disease || id: ebi-a-GCST005194 | MR Egger
(bootstrap) | 168 | 0.015433534 | 0.005990206 | 0.005; 2 | SLE GWAS | 191 | Coronary
artery disease || id: ebi-a-GCST005194 | Simple
median | 168 | 0.023673477 | 0.00399989 | 3.24827E−09; 2 | SLE GWAS | 191 | Coronary
artery disease || id: ebi-a-GCST005194 | Weighted
median | 168 | 0.014217027 | 0.003489893 | 4.62578E−05; 2 | SLE GWAS | 191 | Coronary
artery disease || id: ebi-a-GCST005194 | Penalised weighted
median | 168 | 0.014166589 | 0.003644624 | 0.000101498; 2 | SLE GWAS | 191 | Coronary
artery disease || id: ebi-a-GCST005194 | Inverse variance
weighted | 168 | 0.016421129 | 0.002821886 | 5.91286E−09; 2 | SLE
GWAS | 191 | Coronary artery disease || id: ebi-a-GCST005194 | Inverse variance weighted
(multiplicative random effects) | 168 | 0.016421129 | 0.002821886 | 5.91286E−09; 2 | SLE
GWAS | 191 | Coronary artery disease || id: ebi-a-GCST005194 | Inverse variance weighted
(fixed effects) | 168 | 0.016421129 | 0.002449014 | 2.0113E−11; 2 | SLE
GWAS | 191 | Coronary artery disease || id: ebi-a-GCST005194 | Simple
mode | 168 | 0.029440452 | 0.008189295 | 0.000426746; 2 | SLE GWAS | 191 | Coronary
artery disease || id: ebi-a-GCST005194 | Weighted
mode | 168 | 0.006829808 | 0.006248636 | 0.275964546; 2 | SLE GWAS | 191 | Coronary
artery disease || id: ebi-a-GCST005194 | Weighted mode
(NOME) | 168 | 0.010598249 | 0.004963085 | 0.034184868; 2 | SLE
GWAS | 191 | Coronary artery disease || id: ebi-a-GCST005194 | Simple mode
(NOME) | 168 | 0.029440452 | 0.00672552 | 2.11122E−05; 2 | SLE GWAS | 191 | Coronary
artery disease || id: ebi-a-GCST005194 | Robust adjusted profile score
(RAPS) | 168 | 0.016111755 | 0.002776658 | 6.53061E−09; 2 | SLE GWAS | 191 | Coronary
artery disease || id: ebi-a-
GCST005194 | Raw | 168 | 0.016421129 | 0.002821886 | 2.94649E−08; 2 | SLE
GWAS | 191 | Coronary artery disease || id: ebi-a-GCST005194 | Outlier-
corrected | 166 | 0.015611793 | 0.002619238 | 1.47983E−08; 3 | SLE GWAS | 62 | Coronary
artery disease || id: ebi-a-GCST005194 | Maximum
likelihood | 46 | 0.021945406 | 0.003668241 | 2.20E−09; 3 | SLE GWAS | 62 | Coronary
artery disease || id: ebi-a-GCST005194 | MR
Egger | 46 | 0.016161823 | 0.006989505 | 0.025501065; 3 | SLE GWAS | 62 | Coronary
artery disease || id: ebi-a-GCST005194 | MR Egger
(bootstrap) | 46 | 0.019967466 | 0.007013428 | 0.001; 3 | SLE GWAS | 62 | Coronary artery
disease || id: ebi-a-GCST005194 | Simple
median | 46 | 0.017569785 | 0.0055897 | 0.00167087; 3 | SLE GWAS | 62 | Coronary artery
disease || id: ebi-a-GCST005194 | Weighted
median | 46 | 0.022170472 | 0.004959129 | 7.79866E−06; 3 | SLE GWAS | 62 | Coronary
artery disease || id: ebi-a-GCST005194 | Penalised weighted
median | 46 | 0.022170229 | 0.004846319 | 4.77008E−06; 3 | SLE GWAS | 62 | Coronary
artery disease || id: ebi-a-GCST005194 | Inverse variance
weighted | 46 | 0.021807228 | 0.003613444 | 1.59E−09; 3 | SLE GWAS | 62 | Coronary
artery disease || id: ebi-a-GCST005194 | Inverse variance weighted (multiplicative random
effects) | 46 | 0.021807228 | 0.003590954 | 1.26E−09; 3 | SLE GWAS | 62 | Coronary artery
disease || id: ebi-a-GCST005194 | Inverse variance weighted (fixed
effects) | 46 | 0.021807228 | 0.003613444 | 1.59E−09; 3 | SLE GWAS | 62 | Coronary artery
disease || id: ebi-a-GCST005194 | Simple
mode | 46 | 0.018691291 | 0.006989976 | 0.01040819; 3 | SLE GWAS | 62 | Coronary
artery disease || id: ebi-a-GCST005194 | Weighted
mode | 46 | 0.022492005 | 0.005788196 | 0.000332099; 3 | SLE GWAS | 62 | Coronary
artery disease || id: ebi-a-GCST005194 | Weighted mode
(NOME) | 46 | 0.024392362 | 0.005508755 | 6.00834E−05; 3 | SLE GWAS | 62 | Coronary
artery disease || id: ebi-a-GCST005194 | Simple mode
(NOME) | 46 | 0.018691291 | 0.006878494 | 0.00930847; 3 | SLE GWAS | 62 | Coronary
artery disease || id: ebi-a-GCST005194 | Robust adjusted profile score
(RAPS) | 46 | 0.021415922 | 0.003775109 | 1.40E−08; 3 | SLE GWAS | 62 | Coronary
artery disease || id: ebi-a-GCST005194 | Raw | 46 | 0.021807228 | 0.003590954 | 2.43415E−07;
3 | SLE GWAS | 62 | Coronary artery disease || id: ebi-a-GCST005194 | Outlier-
corrected | NA | NA | NA | NA; 4 | SLE GWAS | 95 | Coronary artery disease || id: ebi-a-
GCST005194 | Maximum likelihood | 84 | 0.038037894 | 0.005917767 | 1.29512E−10;
4 | SLE GWAS | 95 | Coronary artery disease || id: ebi-a-GCST005194 | MR
Egger | 84 | 0.010994234 | 0.015051472 | 0.467201623; 4 | SLE GWAS | 95 | Coronary
artery disease || id: ebi-a-GCST005194 | MR Egger
(bootstrap) | 84 | 0.00559573 | 0.015235927 | 0.367; 4 | SLE GWAS | 95 | Coronary artery
disease || id: ebi-a-GCST005194 | Simple
median | 84 | 0.034223328 | 0.010337977 | 0.00093147; 4 | SLE GWAS | 95 | Coronary
artery disease || id: ebi-a-GCST005194 | Weighted
median | 84 | 0.032267485 | 0.008596177 | 0.000174242; 4 | SLE GWAS | 95 | Coronary
artery disease || id: ebi-a-GCST005194 | Penalised weighted
median | 84 | 0.032399304 | 0.008578914 | 0.000158971; 4 | SLE GWAS | 95 | Coronary
artery disease || id: ebi-a-GCST005194 | Inverse variance
weighted | 84 | 0.031628601 | 0.009769575 | 0.001205992; 4 | SLE GWAS | 95 | Coronary
artery disease || id: ebi-a-GCST005194 | Inverse variance weighted (multiplicative random
effects) | 84 | 0.031628601 | 0.009769575 | 0.001205992; 4 | SLE GWAS | 95 | Coronary
artery disease || id: ebi-a-GCST005194 | Inverse variance weighted (fixed
effects) | 84 | 0.031628601 | 0.005115782 | 6.30725E−10; 4 | SLE GWAS | 95 | Coronary
artery disease || id: ebi-a-GCST005194 | Simple
mode | 84 | 0.029336331 | 0.012090772 | 0.017418026; 4 | SLE GWAS | 95 | Coronary
artery disease || id: ebi-a-GCST005194 | Weighted
mode | 84 | 0.029336331 | 0.010855121 | 0.008343199; 4 | SLE GWAS | 95 | Coronary
artery disease || id: ebi-a-GCST005194 | Weighted mode
(NOME) | 84 | 0.029336331 | 0.00814988 | 0.000541037; 4 | SLE GWAS | 95 | Coronary
artery disease || id: ebi-a-GCST005194 | Simple mode
(NOME) | 84 | 0.029336331 | 0.010711026 | 0.007543176; 4 | SLE GWAS | 95 | Coronary
artery disease || id: ebi-a-GCST005194 | Robust adjusted profile score
(RAPS) | 84 | 0.029832159 | 0.008780649 | 0.000680074; 4 | SLE GWAS | 95 | Coronary
artery disease || id: ebi-a-
GCST005194 | Raw | 84 | 0.031628601 | 0.009769575 | 0.001734149; 4 | SLE
GWAS | 95 | Coronary artery disease || id: ebi-a-GCST005194 | Outlier-
corrected | 74 | 0.01892742 | 0.006798066 | 0.006828596; 5 | SLE GWAS | 143 | Coronary
artery disease || id: ebi-a-GCST005194 | Maximum
likelihood | 124 | 0.01298068 | 0.003090876 | 2.67297E−05; 5 | SLE
GWAS | 143 | Coronary artery disease || id: ebi-a-GCST005194 | MR
Egger | 124 | 0.019824113 | 0.009798175 | 0.04523323; 5 | SLE GWAS | 143 | Coronary
artery disease || id: ebi-a-GCST005194 | MR Egger
(bootstrap) | 124 | 0.007096513 | 0.007243014 | 0.156; 5 | SLE GWAS | 143 | Coronary
artery disease || id: ebi-a-GCST005194 | Simple
median | 124 | 0.02182981 | 0.005073168 | 1.68505E−05; 5 | SLE GWAS | 143 | Coronary
artery disease || id: ebi-a-GCST005194 | Weighted
median | 124 | 0.02066011 | 0.004497244 | 4.34936E−06; 5 | SLE GWAS | 143 | Coronary
artery disease || id: ebi-a-GCST005194 | Penalised weighted
median | 124 | 0.022345517 | 0.004596284 | 1.16412E−06; 5 | SLE GWAS | 143 | Coronary
artery disease || id: ebi-a-GCST005194 | Inverse variance
weighted | 124 | 0.012656512 | 0.004200845 | 0.002588079; 5 | SLE
GWAS | 143 | Coronary artery disease || id: ebi-a-GCST005194 | Inverse variance weighted
(multiplicative random effects) | 124 | 0.012656512 | 0.004200845 | 0.002588079; 5 | SLE
GWAS | 143 | Coronary artery disease || id: ebi-a-GCST005194 | Inverse variance weighted
(fixed effects) | 124 | 0.012656512 | 0.002988961 | 2.29144E−05; 5 | SLE
GWAS | 143 | Coronary artery disease || id: ebi-a-GCST005194 | Simple
mode | 124 | 0.036036704 | 0.009025367 | 0.000111536; 5 | SLE GWAS | 143 | Coronary
artery disease || id: ebi-a-GCST005194 | Weighted
mode | 124 | 0.025987505 | 0.007834899 | 0.001197111; 5 | SLE GWAS | 143 | Coronary
artery disease || id: ebi-a-GCST005194 | Weighted mode
(NOME) | 124 | 0.025987505 | 0.007632019 | 0.000893857; 5 | SLE
GWAS | 143 | Coronary artery disease || id: ebi-a-GCST005194 | Simple mode
(NOME) | 124 | 0.036036704 | 0.008810551 | 7.73274E−05; 5 | SLE
GWAS | 143 | Coronary artery disease || id: ebi-a-GCST005194 | Robust adjusted profile
score (RAPS) | 124 | 0.013864395 | 0.004169568 | 0.000883744; 5 | SLE
GWAS | 143 | Coronary artery disease || id: ebi-a-
GCST005194 | Raw | 124 | 0.012656512 | 0.004200845 | 0.003142874; 5 | SLE
GWAS | 143 | Coronary artery disease || id: ebi-a-GCST005194 | Outlier-
corrected | 121 | 0.011585697 | 0.003520024 | 0.001309647; 6 | SLE
GWAS | 61 | Coronary artery disease || id: ebi-a-GCST005194 | Maximum likelihood | 51
| −0.005355239 | 0.004755843 | 0.260151331; 6 | SLE GWAS | 61 | Coronary artery disease ||
id: ebi-a-GCST005194 | MR Egger | 51 | −0.064532441 | 0.021563123 | 0.004321996;
6 | SLE GWAS | 61 | Coronary artery disease || id: ebi-a-GCST005194 | MR Egger
(bootstrap) | 51 | 0.057514853 | 0.01265526 | 0; 6 | SLE GWAS | 61 | Coronary artery
disease || id: ebi-a-GCST005194 | Simple
median | 51 | 0.019931565 | 0.009776449 | 0.041476716; 6 | SLE GWAS | 61 | Coronary
artery disease || id: ebi-a-GCST005194 | Weighted
median | 51 | 0.02708103 | 0.006686823 | 5.12374E−05; 6 | SLE GWAS | 61 | Coronary
artery disease || id: ebi-a-GCST005194 | Penalised weighted
median | 51 | 0.027999139 | 0.006678299 | 2.7583E−05; 6 | SLE GWAS | 61 | Coronary
artery disease || id: ebi-a-GCST005194 | Inverse variance weighted | 51
| −0.004815657 | 0.009836941 | 0.624453619; 6 | SLE GWAS | 61 | Coronary artery disease ||
id: ebi-a-GCST005194 | Inverse variance weighted (multiplicative random effects) | 51
| −0.004815657 | 0.009836941 | 0.624453619; 6 | SLE GWAS | 61 | Coronary artery disease ||
id: ebi-a-GCST005194 | Inverse variance weighted (fixed effects) | 51
| −0.004815657 | 0.004481328 | 0.282551692; 6 | SLE GWAS | 61 | Coronary artery disease ||
id: ebi-a-GCST005194 | Simple mode | 51 | 0.027156987 | 0.008543687 | 0.002539216;
6 | SLE GWAS | 61 | Coronary artery disease || id: ebi-a-GCST005194 | Weighted
mode | 51 | 0.027156987 | 0.007554761 | 0.000741873; 6 | SLE GWAS | 61 | Coronary
artery disease || id: ebi-a-GCST005194 | Weighted mode
(NOME) | 51 | 0.027156987 | 0.006235674 | 6.592E−05; 6 | SLE GWAS | 61 | Coronary
artery disease || id: ebi-a-GCST005194 | Simple mode
(NOME) | 51 | 0.027156987 | 0.007134285 | 0.000385686; 6 | SLE GWAS | 61 | Coronary
artery disease || id: ebi-a-GCST005194 | Robust adjusted profile score (RAPS) | 51
| −0.054880932 | 0.013058154 | 2.64E−05; 6 | SLE GWAS | 61 | Coronary artery disease ||
id: ebi-a-GCST005194 | Raw | 51 | −0.004815657 | 0.009836941 | 0.626594592; 6 | SLE
GWAS | 61 | Coronary artery disease || id: ebi-a-GCST005194 | Outlier-corrected | 47
| −0.000251302 | 0.00892484 | 0.977658259; 7 | SLE GWAS | 56 | Coronary artery disease ||
id: ebi-a-GCST005194 | Maximum likelihood | 50
| −0.004849829 | 0.00586843 | 0.408561974; 7 | SLE GWAS | 56 | Coronary artery disease ||
id: ebi-a-GCST005194 | MR Egger | 50 | −0.023546351 | 0.015962215 | 0.14670711;
7 | SLE GWAS | 56 | Coronary artery disease || id: ebi-a-GCST005194 | MR Egger
(bootstrap) | 50 | −0.017243272 | 0.011603969 | 0.065; 7 | SLE GWAS | 56 | Coronary
artery disease || id: ebi-a-GCST005194 | Simple
median | 50 | 0.011708373 | 0.012515986 | 0.349544354; 7 | SLE GWAS | 56 | Coronary
artery disease || id: ebi-a-GCST005194 | Weighted median | 50
| −0.005042651 | 0.009370176 | 0.59046678; 7 | SLE GWAS | 56 | Coronary artery disease ||
id: ebi-a-GCST005194 | Penalised weighted median | 50
| −0.005042651 | 0.009462453 | 0.594094765; 7 | SLE GWAS | 56 | Coronary artery disease ||
id: ebi-a-GCST005194 | Inverse variance weighted | 50
| −0.004857434 | 0.009097634 | 0.59339497; 7 | SLE GWAS | 56 | Coronary artery disease ||
id: ebi-a-GCST005194 | Inverse variance weighted (multiplicative random effects) | 50
| −0.004857434 | 0.009097634 | 0.59339497; 7 | SLE GWAS | 56 | Coronary artery disease ||
id: ebi-a-GCST005194 | Inverse variance weighted (fixed effects) | 50
| −0.004857434 | 0.005522487 | 0.379090304; 7 | SLE GWAS | 56 | Coronary artery disease ||
id: ebi-a-GCST005194 | Simple mode | 50 | −0.017920265 | 0.015302221 | 0.247226529;
7 | SLE GWAS | 56 | Coronary artery disease || id: ebi-a-GCST005194 | Weighted
mode | 50 | −0.009200835 | 0.011341255 | 0.421133204; 7 | SLE GWAS | 56 | Coronary
artery disease || id: ebi-a-GCST005194 | Weighted mode (NOME) | 50
| −0.009200835 | 0.008395078 | 0.278445189; 7 | SLE GWAS | 56 | Coronary artery disease ||
id: ebi-a-GCST005194 | Simple mode (NOME) | 50
| −0.017920265 | 0.013806429 | 0.200376012; 7 | SLE GWAS | 56 | Coronary artery disease ||
id: ebi-a-GCST005194 | Robust adjusted profile score (RAPS) | 50
| −0.004879486 | 0.010223981 | 0.633177741; 7 | SLE GWAS | 56 | Coronary artery disease ||
id: ebi-a-GCST005194 | Raw | 50 | −0.004857434 | 0.009097634 | 0.595809029; 7 | SLE
GWAS | 56 | Coronary artery disease || id: ebi-a-GCST005194 | Outlier-corrected | 49
| −0.006305097 | 0.008429829 | 0.458138854; 8 | SLE GWAS | 22 | Coronary artery disease ||
id: ebi-a-GCST005194 | Maximum likelihood | 18 | 0.050306465 | 0.010430299 | 1.41338E−06;
8 | SLE GWAS | 22 | Coronary artery disease || id: ebi-a-GCST005194 | MR
Egger | 18 | 0.081908905 | 0.029477341 | 0.013417549; 8 | SLE GWAS | 22 | Coronary
artery disease || id: ebi-a-GCST005194 | MR Egger
(bootstrap) | 18 | 0.046060594 | 0.02008727 | 0.01; 8 | SLE GWAS | 22 | Coronary artery
disease || id: ebi-a-GCST005194 | Simple
median | 18 | 0.022211483 | 0.0155458 | 0.153068306; 8 | SLE GWAS | 22 | Coronary
artery disease || id: ebi-a-GCST005194 | Weighted
median | 18 | 0.041694866 | 0.014160904 | 0.003236184; 8 | SLE GWAS | 22 | Coronary
artery disease || id: ebi-a-GCST005194 | Penalised weighted
median | 18 | 0.041542391 | 0.01397456 | 0.002951779; 8 | SLE GWAS | 22 | Coronary
artery disease || id: ebi-a-GCST005194 | Inverse variance
weighted | 18 | 0.048969037 | 0.010405884 | 2.5275E−06; 8 | SLE GWAS | 22 | Coronary
artery disease || id: ebi-a-GCST005194 | Inverse variance weighted (multiplicative random
effects) | 18 | 0.048969037 | 0.010405884 | 2.5275E−06; 8 | SLE GWAS | 22 | Coronary
artery disease || id: ebi-a-GCST005194 | Inverse variance weighted (fixed
effects) | 18 | 0.048969037 | 0.009807681 | 5.9471E−07; 8 | SLE GWAS | 22 | Coronary
artery disease || id: ebi-a-GCST005194 | Simple
mode | 18 | 0.012591774 | 0.019458307 | 0.526199445; 8 | SLE GWAS | 22 | Coronary
artery disease || id: ebi-a-GCST005194 | Weighted
mode | 18 | 0.034316045 | 0.016801622 | 0.056936012; 8 | SLE GWAS | 22 | Coronary
artery disease || id: ebi-a-GCST005194 | Weighted mode
(NOME) | 18 | 0.037936757 | 0.014649843 | 0.019087283; 8 | SLE GWAS | 22 | Coronary
artery disease || id: ebi-a-GCST005194 | Simple mode
(NOME) | 18 | 0.012591774 | 0.020071848 | 0.538775028; 8 | SLE GWAS | 22 | Coronary
artery disease || id: ebi-a-GCST005194 | Robust adjusted profile score
(RAPS) | 18 | 0.047420695 | 0.010817754 | 1.16735E−05; 8 | SLE GWAS | 22 | Coronary
artery disease || id: ebi-a-
GCST005194 | Raw | 18 | 0.048969037 | 0.010405884 | 0.000203641; 8 | SLE
GWAS | 22 | Coronary artery disease || id: ebi-a-GCST005194 | Outlier-
corrected | NA | NA | NA | NA; 9 | SLE GWAS | 264 | Coronary artery disease || id: ebi-a-
GCST005194 | Maximum likelihood | 231 | 0.004329378 | 0.001897656 | 0.02252277;
9 | SLE GWAS | 264 | Coronary artery disease || id: ebi-a-GCST005194 | MR Egger | 231
| −0.003506251 | 0.004839623 | 0.469505472; 9 | SLE GWAS | 264 | Coronary artery disease
| id: ebi-a-GCST005194 | MR Egger | (bootstrap) | 231
| −0.012151743 | 0.004939684 | 0.008; 9 | SLE GWAS | 264 | Coronary artery disease ||
id: ebi-a-GCST005194 | Simple median | 231 | 0.008523279 | 0.003243529 | 0.008594413;
9 | SLE GWAS | 264 | Coronary artery disease || id: ebi-a-GCST005194 | Weighted
median | 231 | −0.000675689 | 0.00292899 | 0.817555614; 9 | SLE GWAS | 264 | Coronary
artery disease || id: ebi-a-GCST005194 | Penalised weighted median | 231
| −0.000719118 | 0.002975181 | 0.809008251; 9 | SLE GWAS | 264 | Coronary artery disease
|| id: ebi-a-GCST005194 | Inverse variance
weighted | 231 | 0.004275448 | 0.002560797 | 0.095003068; 9 | SLE
GWAS | 264 | Coronary artery disease || id: ebi-a-GCST005194 | Inverse variance weighted
(multiplicative random effects) | 231 | 0.004275448 | 0.002560797 | 0.095003068; 9 | SLE
GWAS | 264 | Coronary artery disease || id: ebi-a-GCST005194 | Inverse variance weighted
(fixed effects) | 231 | 0.004275448 | 0.001856041 | 0.021248972; 9 | SLE
GWAS | 264 | Coronary artery disease || id: ebi-a-GCST005194 | Simple
mode | 231 | 0.029222351 | 0.006469479 | 1.00285E−05; 9 | SLE GWAS | 264 | Coronary
artery disease || id: ebi-a-GCST005194 | Weighted mode | 231
| −0.000581841 | 0.004417848 | 0.895334877; 9 | SLE GWAS | 264 | Coronary artery disease
|| id: ebi-a-GCST005194 | Weighted mode (NOME) | 231
| −0.000581841 | 0.004145607 | 0.888505341; 9 | SLE GWAS | 264 | Coronary artery disease
|| id: ebi-a-GCST005194 | Simple mode
(NOME) | 231 | 0.029222351 | 0.006817679 | 2.67336E−05; 9 | SLE
GWAS | 264 | Coronary artery disease || id: ebi-a-GCST005194 | Robust adjusted profile
score (RAPS) | 231 | 0.004239736 | 0.002534984 | 0.094427614; 9 | SLE
GWAS | 264 | Coronary artery disease || id: ebi-a-
GCST005194 | Raw | 231 | 0.004275448 | 0.002560797 | 0.096363506; 9 | SLE
GWAS | 264 | Coronary artery disease || id: ebi-a-GCST005194 | Outlier-
corrected | 226 | 0.003006825 | 0.00223585 | 0.180036056; 10 | SLE
GWAS | 94 | Coronary artery disease || id: ebi-a-GCST005194 | Maximum likelihood | 82
| −0.031001073 | 0.005018084 | 6.50E−10; 10 | SLE GWAS | 94 | Coronary artery disease ||
id: ebi-a-GCST005194 | MR Egger | 82 | −0.092287761 | 0.017220511 | 7.86139E−07;
10 | SLE GWAS | 94 | Coronary artery disease || id: ebi-a-GCST005194 | MR Egger
(bootstrap) | 82 | −0.04392945 | 0.018306041 | 0.01; 10 | SLE GWAS | 94 | Coronary artery
disease || id: ebi-a-GCST005194 | Simple median | 82
| −0.018013596 | 0.009018 | 0.045769198; 10 | SLE GWAS | 94 | Coronary artery disease ||
id: ebi-a-GCST005194 | Weighted median | 82 | −0.039676392 | 0.007559388 | 1.53239E−07;
10 | SLE GWAS | 94 | Coronary artery disease || id: ebi-a-GCST005194 | Penalised
weighted median | 82 | −0.040986607 | 0.007480592 | 4.27593E−08; 10 | SLE
GWAS | 94 | Coronary artery disease || id: ebi-a-GCST005194 | Inverse variance
weighted | 82 | −0.029604363 | 0.008008113 | 0.000218337; 10 | SLE
GWAS | 94 | Coronary artery disease || id: ebi-a-GCST005194 | Inverse variance weighted
(multiplicative random effects) | 82 | −0.029604363 | 0.008008113 | 0.000218337; 10 | SLE
GWAS | 94 | Coronary artery disease || id: ebi-a-GCST005194 | Inverse variance weighted
(fixed effects) | 82 | −0.029604363 | 0.004610925 | 1.35843E−10; 10 | SLE
GWAS | 94 | Coronary artery disease || id: ebi-a-GCST005194 | Simple mode | 82
| −0.055807921 | 0.01676699 | 0.001315694; 10 | SLE GWAS | 94 | Coronary artery disease ||
id: ebi-a-GCST005194 | Weighted mode | 82 | −0.064101605 | 0.013863817 | 1.40836E−05;
10 | SLE GWAS | 94 | Coronary artery disease || id: ebi-a-GCST005194 | Weighted mode
(NOME) | 82 | −0.064101605 | 0.010224714 | 1.66494E−08; 10 | SLE
GWAS | 94 | Coronary artery disease || id: ebi-a-GCST005194 | Simple mode
(NOME) | 82 | −0.055807921 | 0.017690952 | 0.002255711; 10 | SLE
GWAS | 94 | Coronary artery disease || id: ebi-a-GCST005194 | Robust adjusted profile
score (RAPS) | 82 | −0.036783469 | 0.008162925 | 6.60105E−06; 10 | SLE
GWAS | 94 | Coronary artery disease || id: ebi-a-GCST005194 | Raw | 82
| −0.029604363 | 0.008008113 | 0.000395798; 10 | SLE GWAS | 94 | Coronary artery disease
| id: ebi-a-GCST005194 | Outlier-corrected | 78 | −0.032684627 | 0.006445191 | 2.65793E−06;
11 | SLE GWAS | 16 | Coronary artery disease || id: ebi-a-GCST005194 | Maximum
likelihood | 14 | 0.029892197 | 0.014884943 | 0.044620226; 11 | SLE
GWAS | 16 | Coronary artery disease || id: ebi-a-GCST005194 | MR
Egger | 14 | 0.001106137 | 0.056736038 | 0.984765698; 11 | SLE GWAS | 16 | Coronary
artery disease || id: ebi-a-GCST005194 | MR Egger | (bootstrap) | 14
| −0.011362478 | 0.034077582 | 0.364; 11 | SLE GWAS | 16 | Coronary artery disease ||
id: ebi-a-GCST005194 | Simple median | 14 | 0.016350957 | 0.029783854 | 0.583014173;
11 | SLE GWAS | 16 | Coronary artery disease || id: ebi-a-GCST005194 | Weighted
median | 14 | 0.007209443 | 0.016658349 | 0.665172757; 11 | SLE GWAS | 16 | Coronary
artery disease || id: ebi-a-GCST005194 | Penalised weighted
median | 14 | 0.007126832 | 0.016711023 | 0.66976184; 11 | SLE GWAS | 16 | Coronary
artery disease || id: ebi-a-GCST005194 | Inverse variance
weighted | 14 | 0.021283189 | 0.029515099 | 0.470851091; 11 | SLE GWAS | 16 | Coronary
artery disease || id: ebi-a-GCST005194 | Inverse variance weighted (multiplicative random
effects) | 14 | 0.021283189 | 0.029515099 | 0.470851091; 11 | SLE GWAS | 16 | Coronary
artery disease || id: ebi-a-GCST005194 | Inverse variance weighted (fixed
effects) | 14 | 0.021283189 | 0.012177406 | 0.08050551; 11 | SLE GWAS | 16 | Coronary
artery disease || id: ebi-a-GCST005194 | Simple
mode | 14 | 0.070512962 | 0.028322184 | 0.027111498; 11 | SLE GWAS | 16 | Coronary
artery disease || id: ebi-a-GCST005194 | Weighted
mode | 14 | 0.003021977 | 0.014830258 | 0.841687613; 11 | SLE GWAS | 16 | Coronary
artery disease || id: ebi-a-GCST005194 | Weighted mode
(NOME) | 14 | 0.006235833 | 0.013842518 | 0.659782749; 11 | SLE GWAS | 16 | Coronary
artery disease || id: ebi-a-GCST005194 | Simple mode
(NOME) | 14 | 0.070512962 | 0.036545782 | 0.075792354; 11 | SLE GWAS | 16 | Coronary
artery disease || id: ebi-a-GCST005194 | Robust adjusted profile score
(RAPS) | 14 | 0.021936808 | 0.031624776 | 0.487896137; 11 | SLE GWAS | 16 | Coronary
artery disease || id: ebi-a-
GCST005194 | Raw | 14 | 0.021283189 | 0.029515099 | 0.483618238; 11 | SLE
GWAS | 16 | Coronary artery disease || id: ebi-a-GCST005194 | Outlier-
corrected | 10 | 0.012030035 | 0.018449332 | 0.53066197; 12 | SLE GWAS | 56 | Coronary
artery disease || id: ebi-a-GCST005194 | Maximum
likelihood | 47 | 0.119556528 | 0.01728226 | 4.58466E−12; 12 | SLE GWAS | 56 | Coronary
artery disease || id: ebi-a-GCST005194 | MR
Egger | 47 | 0.069806155 | 0.040477153 | 0.091466488; 12 | SLE GWAS | 56 | Coronary
artery disease || id: ebi-a-GCST005194 | MR Egger
(bootstrap) | 47 | 0.073564274 | 0.025287275 | 0.005; 12 | SLE GWAS | 56 | Coronary
artery disease || id: ebi-a-GCST005194 | Simple
median | 47 | 0.006034673 | 0.024891905 | 0.808442975; 12 | SLE GWAS | 56 | Coronary
artery disease || id: ebi-a-GCST005194 | Weighted median | 47
| −0.000748073 | 0.016781699 | 0.964444693; 12 | SLE GWAS | 56 | Coronary artery disease
| id: ebi-a-GCST005194 | Penalised weighted median | 47
| −0.013835282 | 0.016678508 | 0.406805955; 12 | SLE GWAS | 56 | Coronary artery disease
| id: ebi-a-GCST005194 | Inverse variance
weighted | 47 | 0.048112414 | 0.02372673 | 0.042583457; 12 | SLE GWAS | 56 | Coronary
artery disease || id: ebi-a-GCST005194 | Inverse variance weighted (multiplicative random
effects) | 47 | 0.048112414 | 0.02372673 | 0.042583457; 12 | SLE GWAS | 56 | Coronary
artery disease || id: ebi-a-GCST005194 | Inverse variance weighted (fixed
effects) | 47 | 0.048112414 | 0.009576359 | 5.05846E−07; 12 | SLE GWAS | 56 | Coronary
artery disease || id: ebi-a-GCST005194 | Simple
mode | 47 | 0.031809754 | 0.027656652 | 0.25601974; 12 | SLE GWAS | 56 | Coronary
artery disease || id: ebi-a-GCST005194 | Weighted mode | 47
| −0.01270927 | 0.013939 | 0.366639443; 12 | SLE GWAS | 56 | Coronary artery disease ||
id: ebi-a-GCST005194 | Weighted mode (NOME) | 47
| −0.01270927 | 0.013287509 | 0.343828546; 12 | SLE GWAS | 56 | Coronary artery disease ||
id: ebi-a-GCST005194 | Simple mode
(NOME) | 47 | 0.031809754 | 0.028697044 | 0.273421834; 12 | SLE GWAS | 56 | Coronary
artery disease || id: ebi-a-GCST005194 | Robust adjusted profile score
(RAPS) | 47 | 0.017588666 | 0.024109007 | 0.465666829; 12 | SLE GWAS | 56 | Coronary
artery disease || id: ebi-a-
GCST005194 | Raw | 47 | 0.048112414 | 0.02372673 | 0.048398788; 12 | SLE
GWAS | 56 | Coronary artery disease || id: ebi-a-GCST005194 | Outlier-
corrected | 39 | 0.002113271 | 0.016369268 | 0.897959509; 13 | SLE
GWAS | 44 | Coronary artery disease || id: ebi-a-GCST005194 | Maximum
likelihood | 37 | 0.03346501 | 0.007589515 | 1.0367E−05; 13 | SLE GWAS | 44 | Coronary
artery disease || id: ebi-a-GCST005194 | MR
Egger | 37 | 0.044843418 | 0.013010303 | 0.001493092; 13 | SLE GWAS | 44 | Coronary
artery disease || id: ebi-a-GCST005194 | MR Egger
(bootstrap) | 37 | 0.030791207 | 0.019056783 | 0.049; 13 | SLE GWAS | 44 | Coronary
artery disease || id: ebi-a-GCST005194 | Simple
median | 37 | 0.037889049 | 0.012956333 | 0.003451596; 13 | SLE GWAS | 44 | Coronary
artery disease || id: ebi-a-GCST005194 | Weighted
median | 37 | 0.037930803 | 0.010416838 | 0.000271268; 13 | SLE GWAS | 44 | Coronary
artery disease || id: ebi-a-GCST005194 | Penalised weighted
median | 37 | 0.037978394 | 0.010309457 | 0.000229746; 13 | SLE GWAS | 44 | Coronary
artery disease || id: ebi-a-GCST005194 | Inverse variance
weighted | 37 | 0.031121478 | 0.008379926 | 0.00020416; 13 | SLE GWAS | 44 | Coronary
artery disease || id: ebi-a-GCST005194 | Inverse variance weighted (multiplicative random
effects) | 37 | 0.031121478 | 0.008379926 | 0.00020416; 13 | SLE GWAS | 44 | Coronary
artery disease || id: ebi-a-GCST005194 | Inverse variance weighted (fixed
effects) | 37 | 0.031121478 | 0.007080913 | 1.1071E−05; 13 | SLE GWAS | 44 | Coronary
artery disease || id: ebi-a-GCST005194 | Simple
mode | 37 | 0.044933299 | 0.014725656 | 0.00426134; 13 | SLE GWAS | 44 | Coronary
artery disease || id: ebi-a-GCST005194 | Weighted
mode | 37 | 0.044933299 | 0.012083212 | 0.000679176; 13 | SLE GWAS | 44 | Coronary
artery disease || id: ebi-a-GCST005194 | Weighted mode
(NOME) | 37 | 0.044933299 | 0.010821475 | 0.000192873; 13 | SLE GWAS | 44 | Coronary
artery disease || id: ebi-a-GCST005194 | Simple mode
(NOME) | 37 | 0.044933299 | 0.013364191 | 0.001844052; 13 | SLE GWAS | 44 | Coronary
artery disease || id: ebi-a-GCST005194 | Robust adjusted profile score
(RAPS) | 37 | 0.037006035 | 0.008620019 | 1.76248E−05; 13 | SLE GWAS | 44 | Coronary
artery disease || id: ebi-a-
GCST005194 | Raw | 37 | 0.031121478 | 0.008379926 | 0.00068862; 13 | SLE
GWAS | 44 | Coronary artery disease || id: ebi-a-GCST005194 | Outlier-
corrected | NA | NA | NA | NA; 14 | SLE GWAS | 96 | Coronary artery disease || id: ebi-a-
GCST005194 | Maximum likelihood | 81 | 0.029070114 | 0.007821993 | 0.000202035;
14 | SLE GWAS | 96 | Coronary artery disease || id: ebi-a-GCST005194 | MR Egger | 81
| −0.033740198 | 0.020735966 | 0.107690945; 14 | SLE GWAS | 96 | Coronary artery disease
|| id: ebi-a-GCST005194 | MR Egger | (bootstrap) | 81 | −0.010893862 | 0.016670988 | 0.257;
14 | SLE GWAS | 96 | Coronary artery disease || id: ebi-a-GCST005194 | Simple
median | 81 | 0.044078184 | 0.014601799 | 0.002538771; 14 | SLE GWAS | 96 | Coronary
artery disease || id: ebi-a-GCST005194 | Weighted
median | 81 | 0.006163824 | 0.01090832 | 0.57203488; 14 | SLE GWAS | 96 | Coronary
artery disease || id: ebi-a-GCST005194 | Penalised weighted
median | 81 | 0.005873589 | 0.01106682 | 0.595599871; 14 | SLE GWAS | 96 | Coronary
artery disease || id: ebi-a-GCST005194 | Inverse variance
weighted | 81 | 0.023879493 | 0.01129836 | 0.034554909; 14 | SLE GWAS | 96 | Coronary
artery disease || id: ebi-a-GCST005194 | Inverse variance weighted (multiplicative random
effects) | 81 | 0.023879493 | 0.01129836 | 0.034554909; 14 | SLE GWAS | 96 | Coronary
artery disease || id: ebi-a-GCST005194 | Inverse variance weighted (fixed
effects) | 81 | 0.023879493 | 0.00668073 | 0.000351054; 14 | SLE GWAS | 96 | Coronary
artery disease || id: ebi-a-GCST005194 | Simple
mode | 81 | 0.039696417 | 0.017944121 | 0.029802751; 14 | SLE GWAS | 96 | Coronary
artery disease || id: ebi-a-GCST005194 | Weighted
mode | 81 | 0.005577054 | 0.014512918 | 0.70178896; 14 | SLE GWAS | 96 | Coronary
artery disease || id: ebi-a-GCST005194 | Weighted mode
(NOME) | 81 | 0.005577054 | 0.012326737 | 0.65217972; 14 | SLE GWAS | 96 | Coronary
artery disease || id: ebi-a-GCST005194 | Simple mode
(NOME) | 81 | 0.039696417 | 0.017769631 | 0.028278005; 14 | SLE GWAS | 96 | Coronary
artery disease || id: ebi-a-GCST005194 | Robust adjusted profile score
(RAPS) | 81 | 0.021883418 | 0.01253924 | 0.080950944; 14 | SLE GWAS | 96 | Coronary
artery disease || id: ebi-a-
GCST005194 | Raw | 81 | 0.023879493 | 0.01129836 | 0.037670966; 14 | SLE
GWAS | 96 | Coronary artery disease || id: ebi-a-GCST005194 | Outlier-
corrected | 76 | 0.012736906 | 0.009182711 | 0.169534094; 15 | SLE GWAS | 6 | Coronary
artery disease || id: ebi-a-GCST005194 | Maximum
likelihood | 4 | 0.086382094 | 0.043626564 | 0.047699672; 15 | SLE GWAS | 6 | Coronary
artery disease || id: ebi-a-GCST005194 | MR
Egger | 4 | 0.073283809 | 0.082603898 | 0.468585591; 15 | SLE GWAS | 6 | Coronary
artery disease || id: ebi-a-GCST005194 | MR Egger
(bootstrap) | 4 | 0.075852096 | 0.135408444 | 0.244; 15 | SLE GWAS | 6 | Coronary artery
disease || id: ebi-a-GCST005194 | Simple
median | 4 | 0.083826967 | 0.104310572 | 0.421611456; 15 | SLE GWAS | 6 | Coronary
artery disease || id: ebi-a-GCST005194 | Weighted
median | 4 | 0.083605432 | 0.056471944 | 0.138745909; 15 | SLE GWAS | 6 | Coronary
artery disease || id: ebi-a-GCST005194 | Penalised weighted
median | 4 | 0.083605432 | 0.062114953 | 0.178309242; 15 | SLE GWAS | 6 | Coronary
artery disease || id: ebi-a-GCST005194 | Inverse variance
weighted | 4 | 0.081469907 | 0.039192013 | 0.037641485; 15 | SLE GWAS | 6 | Coronary
artery disease || id: ebi-a-GCST005194 | Inverse variance weighted (multiplicative random
effects) | 4 | 0.081469907 | 0.032515264 | 0.012224742; 15 | SLE GWAS | 6 | Coronary
artery disease || id: ebi-a-GCST005194 | Inverse variance weighted (fixed
effects) | 4 | 0.081469907 | 0.039192013 | 0.037641485; 15 | SLE GWAS | 6 | Coronary
artery disease || id: ebi-a-GCST005194 | Simple
mode | 4 | 0.07036745 | 0.060300468 | 0.327554703; 15 | SLE GWAS | 6 | Coronary artery
disease || id: ebi-a-GCST005194 | Weighted
mode | 4 | 0.082227423 | 0.045207122 | 0.166502664; 15 | SLE GWAS | 6 | Coronary
artery disease || id: ebi-a-GCST005194 | Weighted mode
(NOME) | 4 | 0.082227423 | 0.042758386 | 0.150183959; 15 | SLE GWAS | 6 | Coronary
artery disease || id: ebi-a-GCST005194 | Simple mode
(NOME) | 4 | 0.07036745 | 0.054083911 | 0.284143107; 15 | SLE GWAS | 6 | Coronary
artery disease || id: ebi-a-GCST005194 | Robust adjusted profile score
(RAPS) | 4 | 0.086375697 | 0.046853293 | 0.0652509; 15 | SLE GWAS | 6 | Coronary
artery disease || id: ebi-a-
GCST005194 | Raw | 4 | 0.081469907 | 0.032515264 | 0.087275745; 15 | SLE
GWAS | 6 | Coronary artery disease || id: ebi-a-GCST005194 | Outlier-
corrected | NA | NA | NA | NA; 16 | SLE GWAS | 265 | Coronary artery disease || id: ebi-a-
GCST005194 | Maximum likelihood | 233 | 0.006173563 | 0.002370206 | 0.009196743;
16 | SLE GWAS | 265 | Coronary artery disease || id: ebi-a-GCST005194 | MR
Egger | 233 | −0.04558586 | 0.010063235 | 9.46068E−06; 16 | SLE GWAS | 265 | Coronary
artery disease || id: ebi-a-GCST005194 | MR Egger | (bootstrap) | 233
| −0.011422425 | 0.009340665 | 0.11; 16 | SLE GWAS | 265 | Coronary artery disease ||
id: ebi-a-GCST005194 | Simple median | 233 | 0.014864829 | 0.004069997 | 0.000259907;
16 | SLE GWAS | 265 | Coronary artery disease || id: ebi-a-GCST005194 | Weighted
median | 233 | 0.002390695 | 0.003724719 | 0.520973295; 16 | SLE
GWAS | 265 | Coronary artery disease || id: ebi-a-GCST005194 | Penalised weighted
median | 233 | 0.002807383 | 0.003898207 | 0.471418611; 16 | SLE
GWAS | 265 | Coronary artery disease || id: ebi-a-GCST005194 | Inverse variance
weighted | 233 | 0.006117102 | 0.00340734 | 0.072610403; 16 | SLE
GWAS | 265 | Coronary artery disease || id: ebi-a-GCST005194 | Inverse variance weighted
(multiplicative random effects) | 233 | 0.006117102 | 0.00340734 | 0.072610403; 16 | SLE
GWAS | 265 | Coronary artery disease || id: ebi-a-GCST005194 | Inverse variance weighted
(fixed effects) | 233 | 0.006117102 | 0.002283132 | 0.007378519; 16 | SLE
GWAS | 265 | Coronary artery disease || id: ebi-a-GCST005194 | Simple
mode | 233 | 0.031544862 | 0.014213976 | 0.027435404; 16 | SLE GWAS | 265 | Coronary
artery disease || id: ebi-a-GCST005194 | Weighted mode | 233
| −0.015487849 | 0.012646535 | 0.221941123; 16 | SLE GWAS | 265 | Coronary artery
disease || id: ebi-a-GCST005194 | Weighted mode (NOME) | 233
| −0.015487849 | 0.014968255 | 0.301880259; 16 | SLE GWAS | 265 | Coronary artery
disease || id: ebi-a-GCST005194 | Simple mode
(NOME) | 233 | 0.031544862 | 0.015614157 | 0.044504464; 16 | SLE
GWAS | 265 | Coronary artery disease || id: ebi-a-GCST005194 | Robust adjusted profile
score (RAPS) | 233 | 0.005021045 | 0.003548336 | 0.157056083; 16 | SLE
GWAS | 265 | Coronary artery disease || id: ebi-a-
GCST005194 | Raw | 233 | 0.006117102 | 0.00340734 | 0.073911861; 16 | SLE
GWAS | 265 | Coronary artery disease || id: ebi-a-GCST005194 | Outlier-
corrected | 229 | 0.004797338 | 0.003168073 | 0.131340712; 17 | SLE
GWAS | 28 | Coronary artery disease || id: ebi-a-GCST005194 | Maximum
likelihood | 22 | 0.021798646 | 0.011772792 | 0.064081529; 17 | SLE
GWAS | 28 | Coronary artery disease || id: ebi-a-GCST005194 | MR
Egger | 22 | 0.023533735 | 0.031325012 | 0.461235875; 17 | SLE GWAS | 28 | Coronary
artery disease || id: ebi-a-GCST005194 | MR Egger
(bootstrap) | 22 | 0.017718352 | 0.025979911 | 0.245; 17 | SLE GWAS | 28 | Coronary
artery disease || id: ebi-a-GCST005194 | Simple
median | 22 | 0.010101357 | 0.019855421 | 0.61093059; 17 | SLE GWAS | 28 | Coronary
artery disease || id: ebi-a-GCST005194 | Weighted
median | 22 | 0.02339029 | 0.017645353 | 0.184979547; 17 | SLE GWAS | 28 | Coronary
artery disease || id: ebi-a-GCST005194 | Penalised weighted
median | 22 | 0.029905562| 0.018167243 | 0.099737924; 17 | SLE GWAS | 28 | Coronary
artery disease || id: ebi-a-GCST005194 | Inverse variance
weighted | 22 | 0.020857404 | 0.014605463 | 0.153276049; 17 | SLE GWAS | 28 | Coronary
artery disease || id: ebi-a-GCST005194 | Inverse variance weighted (multiplicative random
effects) | 22 | 0.020857404 | 0.014605463 | 0.153276049; 17 | SLE GWAS | 28 | Coronary
artery disease || id: ebi-a-GCST005194 | Inverse variance weighted (fixed
effects) | 22 | 0.020857404 | 0.011005889 | 0.058077425; 17 | SLE GWAS | 28 | Coronary
artery disease || id: ebi-a-GCST005194 | Simple
mode | 22 | 0.010612141 | 0.031297071 | 0.73791843; 17 | SLE GWAS | 28 | Coronary
artery disease || id: ebi-a-GCST005194 | Weighted
mode | 22 | 0.047931878 | 0.023742466 | 0.05645523; 17 | SLE GWAS | 28 | Coronary
artery disease || id: ebi-a-GCST005194 | Weighted mode
(NOME) | 22 | 0.053460728 | 0.023270275 | 0.031977033; 17 | SLE GWAS | 28 | Coronary
artery disease || id: ebi-a-GCST005194 | Simple mode
(NOME) | 22 | 0.010612141 | 0.031220477 | 0.737300864; 17 | SLE GWAS | 28 | Coronary
artery disease || id: ebi-a-GCST005194 | Robust adjusted profile score
(RAPS) | 22 | 0.02493327 | 0.015916888 | 0.117239475; 17 | SLE GWAS | 28 | Coronary
artery disease || id: ebi-a-
GCST005194 | Raw | 22 | 0.020857404 | 0.014605463 | 0.167980077; 17 | SLE
GWAS | 28 | Coronary artery disease || id: ebi-a-GCST005194 | Outlier-
corrected | 21 | 0.026836412 | 0.013225936 | 0.055972559; 18 | SLE
GWAS | 27 | Coronary artery disease || id: ebi-a-GCST005194 | Maximum
likelihood | 25 | 0.168492336 | 0.026824252 | 3.3566E−10; 18 | SLE GWAS | 27 | Coronary
artery disease || id: ebi-a-GCST005194 | MR
Egger | 25 | 0.022890419 | 0.067728514 | 0.738448899; 18 | SLE GWAS | 27 | Coronary
artery disease || id: ebi-a-GCST005194 | MR Egger
(bootstrap) | 25 | 0.07141364 | 0.061809945 | 0.122; 18 | SLE GWAS | 27 | Coronary artery
disease || id: ebi-a-GCST005194 | Simple
median | 25 | 0.13072427 | 0.047458287 | 0.005878031; 18 | SLE GWAS | 27 | Coronary
artery disease || id: ebi-a-GCST005194 | Weighted
median | 25 | 0.010723324 | 0.027507982 | 0.696665287; 18 | SLE GWAS | 27 | Coronary
artery disease || id: ebi-a-GCST005194 | Penalised weighted
weighted | 25 | 0.009166981 | 0.026649261 | 0.730856582; 18 | SLE GWAS | 27 | Coronary
artery disease || id: ebi-a-GCST005194 | Inverse variance
weighted | 25 | 0.081021574 | 0.035827378 | 0.023731838; 18 | SLE GWAS | 27 | Coronary
artery disease || id: ebi-a-GCST005194 | Inverse variance weighted (multiplicative random
effects) | 25 | 0.081021574 | 0.035827378 | 0.023731838; 18 | SLE GWAS | 27 | Coronary
artery disease || id: ebi-a-GCST005194 | Inverse variance weighted (fixed
effects) | 25 | 0.081021574 | 0.014024372 | 7.59548E−09; 18 | SLE GWAS | 27 | Coronary
artery disease || id: ebi-a-GCST005194 | Simple
mode | 25 | 0.146655165 | 0.049671385 | 0.006945077; 18 | SLE GWAS | 27 | Coronary
artery disease || id: ebi-a-GCST005194 | Weighted
mode | 25 | 0.01399105 | 0.027418137 | 0.614513558; 18 | SLE GWAS | 27 | Coronary
artery disease || id: ebi-a-GCST005194 | Weighted mode
(NOME) | 25 | 0.032943066 | 0.045255835 | 0.473699289; 18 | SLE GWAS | 27 | Coronary
artery disease || id: ebi-a-GCST005194 | Simple mode
(NOME) | 25 | 0.146655165 | 0.064682999 | 0.03266121; 18 | SLE GWAS | 27 | Coronary
artery disease || id: ebi-a-GCST005194 | Robust adjusted profile score
(RAPS) | 25 | 0.075726281 | 0.03912892 | 0.052953236; 18 | SLE GWAS | 27 | Coronary
artery disease || id: ebi-a-
GCST005194 | Raw | 25 | 0.081021574 | 0.035827378 | 0.033071683; 18 | SLE
GWAS | 27 || Coronary artery disease || id: ebi-a-GCST005194 | Outlier-
corrected | 18 | 0.06953482 | 0.027505579 | 0.021666638; 19 | SLE
GWAS | 216 || Coronary artery disease || id: ebi-a-GCST005194 | Maximum
likelihood | 183 || 0.042462056 | 0.003625047 | 1.08674E−31; 19 | SLE
GWAS | 216 | Coronary artery disease || id: ebi-a-GCST005194 | MR
Egger | 183 | 0.029989823 | 0.009089039 | 0.00116616; 19 | SLE GWAS | 216 | Coronary
artery disease || id: ebi-a-GCST005194 | MR Egger
(bootstrap) | 183 | 0.038028418 | 0.007746113 | 0; 19 | SLE GWAS | 216 | Coronary artery
disease || id: ebi-a-GCST005194 | Simple
median | 183 | 0.031378534 | 0.005610628 | 2.23571E−08; 19 | SLE
GWAS | 216 | Coronary artery disease || id: ebi-a-GCST005194 | Weighted
median | 183 | 0.0290067 | 0.005018355 | 7.46469E−09; 19 | SLE GWAS | 216 | Coronary
artery disease || id: ebi-a-GCST005194 | Penalised weighted
median | 183 | 0.028976883 | 0.005033889 | 8.59461E−09; 19 | SLE
GWAS | 216 | Coronary artery disease || id: ebi-a-GCST005194 | Inverse variance
weighted | 183 | 0.039440412 | 0.005311997 | 1.12967E−13; 19 | SLE
GWAS | 216 | Coronary artery disease || id: ebi-a-GCST005194 | Inverse variance weighted
(multiplicative random effects) | 183 | 0.039440412 | 0.005311997 | 1.12967E−13; 19 | SLE
GWAS | 216 | Coronary artery disease || id: ebi-a-GCST005194 | Inverse variance weighted
(fixed effects) | 183 | 0.039440412 | 0.00329614 | 5.37837E−33; 19 | SLE
GWAS | 216 | Coronary artery disease || id: ebi-a-GCST005194 | Simple
mode | 183 | 0.031144073 | 0.011943768 | 0.009875754; 19 | SLE GWAS | 216 | Coronary
artery disease || id: ebi-a-GCST005194 | Weighted
mode | 183 | 0.031144073 | 0.010623232 | 0.003803943; 19 | SLE GWAS | 216 | Coronary
artery disease || id: ebi-a-GCST005194 | Weighted mode
(NOME) | 183 | 0.031144073 | 0.005018503 | 3.57902E−09; 19 | SLE
GWAS | 216 | Coronary artery disease || id: ebi-a-GCST005194 | Simple mode
(NOME) | 183 | 0.031144073 | 0.006650608 | 5.51538E−06; 19 | SLE
GWAS | 216 | Coronary artery disease || id: ebi-a-GCST005194 | Robust adjusted profile
score (RAPS) | 183 | 0.035382829 | 0.004931942 | 7.27217E−13; 19 | SLE
GWAS | 216 | Coronary artery disease || id: ebi-a-
GCST005194 | Raw | 183 | 0.039440412 | 0.005311997 | 4.20538E−12; 19 | SLE
GWAS | 216 | Coronary artery disease || id: ebi-a-GCST005194 | Outlier-
corrected | 177 | 0.032961089 | 0.004122153 | 1.64346E−13; 20 | SLE
GWAS | 40 | Coronary artery disease || id: ebi-a-GCST005194 | Maximum
likelihood | 33 | 0.008252411 | 0.005290463 | 0.118791662; 20 | SLE
GWAS | 40 | Coronary artery disease || id: ebi-a-GCST005194 | MR Egger | 33
| −0.000636613 | 0.00693499 | 0.927449732; 20 | SLE GWAS | 40 | Coronary artery disease ||
id: ebi-a-GCST005194 | MR Egger | (bootstrap) | 33 | 0.001366288 | 0.00922926 | 0.451;
20 | SLE GWAS | 40 | Coronary artery disease || id: ebi-a-GCST005194 | Simple
median | 33 | 0.00738558 | 0.012972436 | 0.569133113; 20 | SLE GWAS | 40 | Coronary
artery disease || id: ebi-a-GCST005194 | Weighted
median | 33 | 0.003524081 | 0.006514333 | 0.588525867; 20 | SLE GWAS | 40 | Coronary
artery disease || id: ebi-a-GCST005194 | Penalised weighted
median | 33 | 0.00352281 | 0.006404147 | 0.582262723; 20 | SLE GWAS | 40 | Coronary
artery disease || id: ebi-a-GCST005194 | Inverse variance
weighted | 33 | 0.008150189 | 0.00586579 | 0.164697634; 20 | SLE GWAS | 40 | Coronary
artery disease || id: ebi-a-GCST005194 | Inverse variance weighted (multiplicative random
effects) | 33 | 0.008150189 | 0.00586579 | 0.164697634; 20 | SLE GWAS | 40 | Coronary
artery disease || id: ebi-a-GCST005194 | Inverse variance weighted (fixed
effects) | 33 | 0.008150189 | 0.005199711 | 0.117013939; 20 | SLE GWAS | 40 | Coronary
artery disease || id: ebi-a-GCST005194 | Simple
mode | 33 | 0.01945133 | 0.015707247 | 0.224588274; 20 | SLE GWAS | 40 | Coronary
artery disease || id: ebi-a-GCST005194 | Weighted
mode | 33 | 0.003192664 | 0.010481315 | 0.762639041; 20 | SLE GWAS | 40 | Coronary
artery disease || id: ebi-a-GCST005194 | Weighted mode
(NOME) | 33 | 0.003192664 | 0.005747958 | 0.582454173; 20 | SLE GWAS | 40 | Coronary
artery disease || id: ebi-a-GCST005194 | Simple mode
(NOME) | 33 | 0.01945133 | 0.012893003 | 0.141193275; 20 | SLE GWAS | 40 | Coronary
artery disease || id: ebi-a-GCST005194 | Robust adjusted profile score
(RAPS) | 33 | 0.00742878 | 0.006210132 | 0.231604681; 20 | SLE GWAS | 40 | Coronary
artery disease || id: ebi-a-
GCST005194 | Raw | 33 | 0.008150189 | 0.00586579 | 0.174290506; 20 | SLE
GWAS | 40 | Coronary artery disease || id: ebi-a-GCST005194 | Outlier-
corrected | NA | NA | NA | NA; 21 | SLE GWAS | 133 | Coronary artery disease || id: ebi-a-
GCST005194 | Maximum likelihood | 111 | 0.022197905 | 0.003780252 | 4.30389E−09;
21 | SLE GWAS | 133 | Coronary artery disease || id: ebi-a-GCST005194 | MR
Egger | 111 | 0.021921391 | 0.008359408 | 0.009981451; 21 | SLE GWAS | 133 | Coronary
artery disease || id: ebi-a-GCST005194 | MR Egger | (bootstrap) | 111 | 1.84E−02
| 0.008216929 | 0.01; 21 | SLE GWAS | 133 | Coronary artery disease || id: ebi-a-
GCST005194 | Simple median | 111 | 0.027152662 | 0.005621362 | 1.36352E−06; 21 | SLE
GWAS | 133 | Coronary artery disease || id: ebi-a-GCST005194 | Weighted
median | 111 | 0.026759371 | 0.004882805 | 4.24538E−08; 21 | SLE
GWAS | 133 | Coronary artery disease || id: ebi-a-GCST005194 | Penalised weighted
median | 111| 0.026808658 | 0.005030954 | 9.88951E−08; 21 | SLE
GWAS | 133 | Coronary artery disease || id: ebi-a-GCST005194 | Inverse variance
weighted | 111 | 0.021756255 | 0.003691773 | 3.78852E−09; 21 | SLE
GWAS | 133 | Coronary artery disease || id: ebi-a-GCST005194 | Inverse variance weighted
(multiplicative random effects) | 111 | 0.021756255 | 0.003626054 | 1.9734E−09; 21 | SLE
GWAS | 133 | Coronary artery disease || id: ebi-a-GCST005194 | Inverse variance weighted
(fixed effects) | 111 | 0.021756255 | 0.003691773 | 3.78852E−09; 21 | SLE
GWAS | 133 | Coronary artery disease || id: ebi-a-GCST005194 | Simple
mode | 111 | 0.028811361 | 0.010558449 | 0.007403432; 21 | SLE GWAS | 133 | Coronary
artery disease || id: ebi-a-GCST005194 | Weighted
mode | 111 | 0.028811361 | 0.009518501 | 0.003077827; 21 | SLE GWAS | 133 | Coronary
artery disease || id: ebi-a-GCST005194 | Weighted mode
(NOME) | 111 | 0.028811361 | 0.007193148 | 0.000112842; 21 | SLE
GWAS | 133 | Coronary artery disease || id: ebi-a-GCST005194 | Simple mode
(NOME) | 111 | 0.028811361 | 0.008314918 | 0.000756928; 21 | SLE
GWAS | 133 | Coronary artery disease || id: ebi-a-GCST005194 | Robust adjusted profile
score (RAPS) | 111 | 0.023101737 | 0.003954061 | 5.14124E−09; 21 | SLE
GWAS | 133 | Coronary artery disease || id: ebi-a-
GCST005194 | Raw | 111 | 0.021756255 | 0.003626054 | 2.57256E−08; 21 | SLE
GWAS | 133 | Coronary artery disease || id: ebi-a-GCST005194 | Outlier-
corrected | NA | NA | NA | NA; 22 | SLE GWAS | 10 | Coronary artery disease || id: ebi-a-
GCST005194 | Maximum likelihood | 8 | 0.081934561 | 0.052031926 | 0.115326206;
22 | SLE GWAS | 10 | Coronary artery disease || id: ebi-a-GCST005194 | MR
Egger | 8 | 0.060289831 | 0.0628103 | 0.374186918; 22 | SLE GWAS | 10 | Coronary artery
disease || id: ebi-a-GCST005194 | MR Egger
(bootstrap) | 8 | 0.045527317 | 0.070869115 | 0.239; 22 | SLE GWAS | 10 | Coronary artery
disease || id: ebi-a-GCST005194 | Simple
median | 8 | 0.058371013 | 0.091703747 | 0.524439369; 22 | SLE GWAS | 10 | Coronary
artery disease || id: ebi-a-GCST005194 | Weighted
median | 8 | 0.058908848 | 0.068803506 | 0.391892901; 22 | SLE GWAS | 10 | Coronary
artery disease || id: ebi-a-GCST005194 | Penalised weighted
median | 8 | 0.058908848 | 0.07279247 | 0.418359183; 22 | SLE GWAS | 10 | Coronary
artery disease || id: ebi-a-GCST005194 | Inverse variance
weighted | 8 | 0.069530278 | 0.042707716 | 0.103514382; 22 | SLE GWAS | 10 | Coronary
artery disease || id: ebi-a-GCST005194 | Inverse variance weighted (multiplicative random
effects) | 8 | 0.069530278 | 0.033581873 | 0.038408305; 22 | SLE GWAS | 10 | Coronary
artery disease || id: ebi-a-GCST005194 | Inverse variance weighted (fixed
effects) | 8 | 0.069530278 | 0.042707716 | 0.103514382; 22 | SLE GWAS | 10 | Coronary
artery disease || id: ebi-a-GCST005194 | Simple
mode | 8 | 0.049731859 | 0.103101984 | 0.644270581; 22 | SLE GWAS | 10 | Coronary
artery disease || id: ebi-a-GCST005194 | Weighted
mode | 8 | 0.058510952 | 0.0489957 | 0.271289917; 22 | SLE GWAS | 10 | Coronary artery
disease || id: ebi-a-GCST005194 | Weighted mode
(NOME) | 8 | 0.058510952 | 0.043711154 | 0.222546224; 22 | SLE GWAS | 10 | Coronary
artery disease || id: ebi-a-GCST005194 | Simple mode
(NOME) | 8 | 0.049731859 | 0.092950245 | 0.609191962; 22 | SLE GWAS | 10 | Coronary
artery disease || id: ebi-a-GCST005194 | Robust adjusted profile score
(RAPS) | 8 | 0.075600479 | 0.0537849 | 0.159840595; 22 | SLE GWAS | 10 | Coronary
artery disease || id: ebi-a-
GCST005194 | Raw | 8 | 0.069530278 | 0.033581873 | 0.077163136; 22 | SLE
GWAS | 10 | Coronary artery disease || id: ebi-a-GCST005194 | Outlier-
corrected NA | NA | NA | NA; 23 | SLE GWAS | 14 | Coronary artery disease || id: ebi-a-
GCST005194 | Maximum likelihood | 13 | 0.101953878 | 0.020167991 | 4.29867E−07;
23 | SLE GWAS | 14 | Coronary artery disease || id: ebi-a-GCST005194 | MR
Egger | 13 | 0.029130559 | 0.071747186 | 0.692516725; 23 | SLE GWAS | 14 | Coronary
artery disease || id: ebi-a-GCST005194 | MR Egger
(bootstrap) | 13 | 0.155619158 | 0.052368357 | 0.003; 23 | SLE GWAS | 14 | Coronary
artery disease || id: ebi-a-GCST005194 | Simple
median | 13 | 0.072079607 | 0.037183269 | 0.052562776; 23 | SLE GWAS | 14 | Coronary
artery disease || id: ebi-a-GCST005194 | Weighted
median | 13 | 0.009028788 | 0.024571427 | 0.713282878; 23 | SLE GWAS | 14 | Coronary
artery disease || id: ebi-a-GCST005194 | Penalised weighted
median | 13 | 0.007606574 | 0.025198386 | 0.762753053; 23 | SLE GWAS | 14 | Coronary
artery disease || id: ebi-a-GCST005194 | Inverse variance
weighted | 13 | 0.076985929 | 0.030944868 | 0.012852096; 23 | SLE GWAS | 14 | Coronary
artery disease || id: ebi-a-GCST005194 | Inverse variance weighted (multiplicative random
effects) | 13 | 0.076985929 | 0.030944868 | 0.012852096; 23 | SLE GWAS | 14 | Coronary
artery disease || id: ebi-a-GCST005194 | Inverse variance weighted (fixed
effects) | 13 | 0.076985929 | 0.015772351 | 1.05512E−06; 23 | SLE GWAS | 14 | Coronary
artery disease || id: ebi-a-GCST005194 | Simple
mode | 13 | 0.035942179 | 0.037765534 | 0.360016122; 23 | SLE GWAS | 14 | Coronary
artery disease || id: ebi-a-GCST005194 | Weighted
mode | 13 | 0.008892222 | 0.024392617 | 0.721793771; 23 | SLE GWAS | 14 | Coronary
artery disease || id: ebi-a-GCST005194 | Weighted mode
(NOME) | 13 | 0.008892222 | 0.026090285 | 0.739128164; 23 | SLE GWAS | 14 | Coronary
artery disease || id: ebi-a-GCST005194 | Simple mode
(NOME) | 13 | 0.035942179 | 0.040853791 | 0.396257541; 23 | SLE GWAS | 14 | Coronary
artery disease || id: ebi-a-GCST005194 | Robust adjusted profile score
(RAPS) | 13 | 0.047877836 | 0.029936621 | 0.109752495; 23 | SLE GWAS | 14 | Coronary
artery disease || id: ebi-a-
GCST005194 | Raw | 13 | 0.076985929 | 0.030944868 | 0.028544842; 23 | SLE
GWAS | 14 | Coronary artery disease || id: ebi-a-GCST005194 | Outlier-
corrected | 11 | 0.017933763 | 0.0178097 | 0.337694874; 24 | SLE GWAS | 27 | Coronary
artery disease || id: ebi-a-GCST005194 | Maximum likelihood | 20
| −0.128752757 | 0.011966721 | 5.36E−27; 24 | SLE GWAS | 27 | Coronary artery disease ||
id: ebi-a-GCST005194 | MR Egger | 20 | −0.12920744 | 0.017260242 | 6.2286E−07;
24 | SLE GWAS | 27 | Coronary artery disease || id: ebi-a-GCST005194 | MR Egger
(bootstrap) | 20 | −0.069229833 | 0.062763081 | 0.11; 24 | SLE GWAS | 27 | Coronary
artery disease || id: ebi-a-GCST005194 | Simple median | 20
| −0.133840178 | 0.016018583 | 6.52627E−17; 24 | SLE GWAS | 27 | Coronary artery disease
|| id: ebi-a-GCST005194 | Weighted median | 20 | −0.133708623 | 0.014753447 | 1.27059E−19;
24 | SLE GWAS | 27 | Coronary artery disease || id: ebi-a-GCST005194 | Penalised
weighted median | 20 | −0.133708623 | 0.015269175 | 2.00929E−18; 24 | SLE
GWAS | 27 | Coronary artery disease || id: ebi-a-GCST005194 | Inverse variance
weighted | 20 | −0.127415345 | 0.010019122 | 4.74E−37; 24 | SLE GWAS | 27 | Coronary
artery disease || id: ebi-a-GCST005194 | Inverse variance weighted (multiplicative random
effects) | 20 | −0.127415345 | 0.004415103 | 3.91E−183; 24 | SLE GWAS | 27 | Coronary
artery disease || id: ebi-a-GCST005194 | Inverse variance weighted (fixed effects) | 20
| −0.127415345 | 0.010019122 | 4.74E−37; 24 | SLE GWAS | 27 | Coronary artery disease ||
id: ebi-a-GCST005194 | Simple mode | 20 | −0.13436559 | 0.022244082 | 8.24021E−06;
24 | SLE GWAS | 27 | Coronary artery disease || id: ebi-a-GCST005194 | Weighted
mode | 20 | −0.13436559 | 0.022248384 | 8.26062E−06; 24 | SLE GWAS | 27 | Coronary
artery disease || id: ebi-a-GCST005194 | Weighted mode (NOME) | 20
| −0.13436559 | 0.018341786 | 6.04174E−07; 24 | SLE GWAS | 27 | Coronary artery disease ||
id: ebi-a-GCST005194 | Simple mode (NOME) | 20
| −0.13436559 | 0.017556016 | 3.21427E−07; 24 | SLE GWAS | 27 | Coronary artery disease ||
id: ebi-a-GCST005194 | Robust adjusted profile score (RAPS) | 20
| −0.128625969 | 0.012278076 | 1.11E−25; 24 | SLE GWAS | 27 | Coronary artery disease ||
id: ebi-a-GCST005194 | Raw | 20 | −0.127415345 | 0.004415103 | 3.72422E−17; 24 | SLE
GWAS | 27 | Coronary artery disease || id: ebi-a-GCST005194 | Outlier-
corrected | NA | NA | NA | NA; 25 | SLE GWAS | 51 | Coronary artery disease || id: ebi-a-
GCST005194 | Maximum likelihood | 42 | −0.044670285 | 0.007491835 | 2.48363E−09;
25 | SLE GWAS | 51 | Coronary artery disease || id: ebi-a-GCST005194 | MR Egger | 42
| −0.07040739 | 0.022582255 | 0.003369688; 25 | SLE GWAS | 51 | Coronary artery disease ||
id: ebi-a-GCST005194 | MR Egger | (bootstrap) | 42 | 0.034430089 | 0.013835837 | 0.004;
25 | SLE GWAS | 51 | Coronary artery disease || id: ebi-a-GCST005194 | Simple
median | 42 | −0.111364268 | 0.017586271 | 2.41291E−10; 25 | SLE GWAS | 51 | Coronary
artery disease || id: ebi-a-GCST005194 | Weighted
median | 42 | 0.007016224 | 0.013456475 | 0.602087235; 25 | SLE GWAS | 51 | Coronary
artery disease || id: ebi-a-GCST005194 | Penalised weighted
median | 42 | 0.015737564 | 0.011558624 | 0.173342171; 25 | SLE GWAS | 51 | Coronary
artery disease || id: ebi-a-GCST005194 | Inverse variance weighted | 42
| −0.036975651 | 0.014893242 | 0.013038594; 25 | SLE GWAS | 51 | Coronary artery disease
|| id: ebi-a-GCST005194 | Inverse variance weighted (multiplicative random effects) | 42
| −0.036975651 | 0.014893242 | 0.013038594; 25 | SLE GWAS | 51 | Coronary artery disease
|| id: ebi-a-GCST005194 | Inverse variance weighted (fixed effects) | 42
| −0.036975651 | 0.006482294 | 1.16959E−08; 25 | SLE GWAS | 51 | Coronary artery disease
|| id: ebi-a-GCST005194 | Simple mode | 42 | −0.128174067 | 0.021596716 | 5.3551E−07;
25 | SLE GWAS | 51 | Coronary artery disease || id: ebi-a-GCST005194 | Weighted
mode | 42 | 0.015756339 | 0.011348139 | 0.172497495; 25 | SLE GWAS | 51 | Coronary
artery disease || id: ebi-a-GCST005194 | Weighted mode
(NOME) | 42 | 0.01287773 | 0.013163549 | 0.333669757; 25 | SLE GWAS | 51 | Coronary
artery disease || id: ebi-a-GCST005194 | Simple mode (NOME) | 42
| −0.128174067 | 0.015301287 | 2.0254E−10; 25 | SLE GWAS | 51 | Coronary artery disease ||
id: ebi-a-GCST005194 | Robust adjusted profile score (RAPS) | 42
| −0.096629135 | 0.014421627 | 2.08001E−11; 25 | SLE GWAS | 51 | Coronary artery disease
|| id: ebi-a-GCST005194 | Raw | 42 | −0.036975651 | 0.014893242 | 0.017222901; 25 | SLE
GWAS | 51 | Coronary artery disease || id: ebi-a-GCST005194 | Outlier-corrected | 36
| −0.0806993 | 0.013018406 | 4.2278E−07; 26 | SLE GWAS | 2 | Coronary artery disease ||
id: ebi-a-GCST005194 | Robust adjusted profile score (RAPS) | 1
| −0.053273707 | 0.078373152 | 0.496666346; 27 | SLE GWAS | 28 | Coronary artery disease
|| id: ebi-a-GCST005194 | Maximum
likelihood | 19 | 0.038955811 | 0.015591862 | 0.012473044; 27 | SLE
GWAS | 28 | Coronary artery disease || id: ebi-a-GCST005194 | MR
Egger | 19 | 0.031955893 | 0.025982859 | 0.235490194; 27 | SLE GWAS | 28 | Coronary
artery disease || id: ebi-a-GCST005194 | MR Egger
(bootstrap) | 19 | 0.086934786 | 0.040893962 | 0.017; 27 | SLE GWAS | 28 | Coronary
artery disease || id: ebi-a-GCST005194 | Simple median | 19
| −0.007848553 | 0.037811383 | 0.835563818; 27 | SLE GWAS | 28 | Coronary artery disease
|| id: ebi-a-GCST005194 | Weighted median | 19
| −0.009051582 | 0.02279835 | 0.691346851; 27 | SLE GWAS | 28 | Coronary artery disease ||
id: ebi-a-GCST005194 | Penalised weighted median | 19
| −0.013085187 | 0.021380825 | 0.540533985; 27 | SLE GWAS | 28 | Coronary artery disease
|| id: ebi-a-GCST005194 | Inverse variance
weighted | 19 | 0.035039446 | 0.020434769 | 0.086400709; 27 | SLE GWAS | 28 | Coronary
artery disease || id: ebi-a-GCST005194 | Inverse variance weighted (multiplicative random
effects) | 19 | 0.035039446 | 0.020434769 | 0.086400709; 27 | SLE GWAS | 28 | Coronary
artery disease || id: ebi-a-GCST005194 | Inverse variance weighted (fixed
effects) | 19 | 0.035039446 | 0.014154243 | 0.013303351; 27 | SLE GWAS | 28 | Coronary
artery disease || id: ebi-a-GCST005194 | Simple mode | 19
| −0.008334034 | 0.047854436 | 0.863688281; 27 | SLE GWAS | 28 | Coronary artery disease
|| id: ebi-a-GCST005194 | Weighted mode | 19 | 0.005926165 | 0.021657736 | 0.787485516;
27 | SLE GWAS | 28 | Coronary artery disease || id: ebi-a-GCST005194 | Weighted mode
(NOME) | 19 | 0.017809664 | 0.024884757 | 0.483366994; 27 | SLE GWAS | 28 | Coronary
artery disease || id: ebi-a-GCST005194 | Simple mode (NOME) | 19
| −0.008334034 | 0.045112261 | 0.85549857; 27 | SLE GWAS | 28 | Coronary artery disease ||
id: ebi-a-GCST005194 | Robust adjusted profile score
(RAPS) | 19 | 0.013251028 | 0.017102514 | 0.438457843; 27 | SLE GWAS | 28 | Coronary
artery disease || id: ebi-a-
GCST005194 | Raw | 19 | 0.035039446 | 0.020434769 | 0.103566022; 27 | SLE
GWAS | 28 | Coronary artery disease || id: ebi-a-GCST005194 | Outlier-corrected | 18
| −0.00316232 | 0.017565755 | 0.859259442; 28 | SLE GWAS | 27 | Coronary artery disease ||
id: ebi-a-GCST005194 | Maximum likelihood | 23 | 0.252460139 | 0.034074392 | 1.27172E−13;
28 | SLE GWAS | 27 | Coronary artery disease || id: ebi-a-GCST005194 | MR
Egger | 23 | 0.162248325 | 0.087251852 | 0.077018639; 28 | SLE GWAS | 27 | Coronary
artery disease || id: ebi-a-GCST005194 | MR Egger
(bootstrap) | 23 | 0.17740224 | 0.052134607 | 0.001; 28 | SLE GWAS | 27 | Coronary artery
disease || id: ebi-a-GCST005194 | Simple
median | 23 | 0.111514424 | 0.060177786 | 0.063870482; 28 | SLE GWAS | 27 | Coronary
artery disease || id: ebi-a-GCST005194 | Weighted
median | 23 | 0.073870346 | 0.045010708 | 0.100761568; 28 | SLE GWAS | 27 | Coronary
artery disease || id: ebi-a-GCST005194 | Penalised weighted
median | 23 | 0.112583271 | 0.043713397 | 0.010009913; 28 | SLE GWAS | 27 | Coronary
artery disease || id: ebi-a-GCST005194 | Inverse variance
weighted | 23 | 0.133120243 | 0.044894008 | 0.003024746; 28 | SLE GWAS | 27 | Coronary
artery disease || id: ebi-a-GCST005194 | Inverse variance weighted (multiplicative random
effects) | 23 | 0.133120243 | 0.044894008 | 0.003024746; 28 | SLE GWAS | 27 | Coronary
artery disease || id: ebi-a-GCST005194 | Inverse variance weighted (fixed
effects) | 23 | 0.133120243 | 0.016692326 | 1.52459E−15; 28 | SLE GWAS | 27 | Coronary
artery disease || id: ebi-a-GCST005194 | Simple
mode | 23 | 0.071251129 | 0.070413801 | 0.322590247; 28 | SLE GWAS | 27 | Coronary
artery disease || id: ebi-a-GCST005194 | Weighted
mode | 23 | 0.084151115 | 0.048072276 | 0.093964765; 28 | SLE GWAS | 27 | Coronary
artery disease || id: ebi-a-GCST005194 | Weighted mode
(NOME) | 23 | 0.180901011 | 0.034328742 | 2.74898E−05; 28 | SLE GWAS | 27 | Coronary
artery disease || id: ebi-a-GCST005194 | Simple mode
(NOME) | 23 | 0.071251129 | 0.077042676 | 0.365089874; 28 | SLE GWAS | 27 | Coronary
artery disease || id: ebi-a-GCST005194 | Robust adjusted profile score
(RAPS) | 23 | 0.14259412 | 0.058804593 | 0.01531342; 28 | SLE GWAS | 27 | Coronary
artery disease || id: ebi-a-
GCST005194 | Raw | 23 | 0.133120243 | 0.044894008 | 0.007147377; 28 | SLE
GWAS | 27 | Coronary artery disease || id: ebi-a-GCST005194 | Outlier-
corrected | 19 | 0.103670561 | 0.036926015 | 0.01164743; 29 | SLE GWAS | 12 | Coronary
artery disease || id: ebi-a-GCST005194 | Maximum likelihood | 9
| −0.055639256 | 0.026284898 | 0.034278818; 29 | SLE GWAS | 12 | Coronary artery disease
|| id: ebi-a-GCST005194 | MR Egger | 9 | −0.100568311 | 0.087103121 | 0.286147836;
29 | SLE GWAS | 12 | Coronary artery disease || id: ebi-a-GCST005194 | MR Egger
(bootstrap) | 9 | −0.02594045 | 0.077892944 | 0.342; 29 | SLE GWAS | 12 | Coronary artery
disease || id: ebi-a-GCST005194 | Simple median | 9
| −0.019726682 | 0.053629012 | 0.712995517; 29 | SLE GWAS | 12 | Coronary artery disease
|| id: ebi-a-GCST005194 | Weighted median | 9
| −0.006176295 | 0.028927136 | 0.830927514; 29 | SLE GWAS | 12 | Coronary artery disease
|| id: ebi-a-GCST005194 | Penalised weighted median | 9
| −0.005266588 | 0.027210987 | 0.846531079; 29 | SLE GWAS | 12 | Coronary artery disease
|| id: ebi-a-GCST005194 | Inverse variance weighted | 9
| −0.039408903 | 0.051085507 | 0.440451978; 29 | SLE GWAS | 12 | Coronary artery disease
|| id: ebi-a-GCST005194 | Inverse variance weighted (multiplicative random effects) | 9
| −0.039408903 | 0.051085507 | 0.440451978; 29 | SLE GWAS | 12 | Coronary artery disease
|| id: ebi-a-GCST005194 | Inverse variance weighted (fixed effects) | 9
| −0.039408903 | 0.021266725 | 0.063871167; 29 | SLE GWAS | 12 | Coronary artery disease
|| id: ebi-a-GCST005194 | Simple mode | 9 | −0.064630713 | 0.050540841 | 0.236819996;
29 | SLE GWAS | 12 | Coronary artery disease || id: ebi-a-GCST005194 | Weighted
mode | 9 | −0.014591784 | 0.024954386 | 0.57484; 29 | SLE GWAS | 12 | Coronary artery
disease || id: ebi-a-GCST005194 | Weighted mode (NOME) | 9
| −0.031271427 | 0.026766558 | 0.27631971; 29 | SLE GWAS | 12 | Coronary artery disease ||
id: ebi-a-GCST005194 | Simple mode (NOME) | 9
| −0.064630713 | 0.047241475 | 0.208471976; 29 | SLE GWAS | 12 | Coronary artery disease
|| id: ebi-a-GCST005194 | Robust adjusted profile score (RAPS) | 9
| −0.047081887 | 0.045416146 | 0.299886278; 29 | SLE GWAS | 12 | Coronary artery disease
|| id: ebi-a-GCST005194 | Raw | 9 | −0.039408903 | 0.051085507 | 0.462626925; 29 | SLE
GWAS | 12 | Coronary artery disease || id: ebi-a-GCST005194 | Outlier-corrected | 7
| −0.105348237 | 0.027682774 | 0.00890977; 30 | SLE GWAS | 69 | Coronary artery disease ||
id: ebi-a-GCST005194 | Maximum likelihood | 59
| −0.083545935 | 0.005721351 | 2.70795E−48; 30 | SLE GWAS | 69 | Coronary artery disease
|| id: ebi-a-GCST005194 | MR Egger | 59 | −0.132691663 | 0.01486826 | 2.05146E−12;
30 | SLE GWAS | 69 | Coronary artery disease || id: ebi-a-GCST005194 | MR Egger
(bootstrap) | 59 | −0.060499401 | 0.014738538 | 0; 30 | SLE GWAS | 69 | Coronary artery
disease || id: ebi-a-GCST005194 | Simple median | 59
| −0.077080426 | 0.008068693 | 1.25963E−21; 30 | SLE GWAS | 69 | Coronary artery disease
|| id: ebi-a-GCST005194 | Weighted median | 59 | −0.073845824 | 0.007539107 | 1.18255E−22;
30 | SLE GWAS | 69 | Coronary artery disease || id: ebi-a-GCST005194 | Penalised
weighted median | 59 | −0.073929631 | 0.007648298 | 4.19857E−22; 30 | SLE
GWAS | 69 | Coronary artery disease || id: ebi-a-GCST005194 | Inverse variance
weighted | 59 | −0.077299804 | 0.007332911 | 5.56109E−26; 30 | SLE
GWAS | 69 | Coronary artery disease || id: ebi-a-GCST005194 | Inverse variance weighted
(multiplicative random effects) | 59 | −0.077299804 | 0.007332911 | 5.56109E−26; 30 | SLE
GWAS | 69 | Coronary artery disease || id: ebi-a-GCST005194 | Inverse variance weighted
(fixed effects) | 59 | −0.077299804 | 0.005001911 | 7.08435E−54; 30 | SLE
GWAS | 69 | Coronary artery disease || id: ebi-a-GCST005194 | Simple mode | 59
| −0.075066854 | 0.013988046 | 1.46827E−06; 30 | SLE GWAS | 69 | Coronary artery disease
|| id: ebi-a-GCST005194 | Weighted mode | 59 | −0.071829917 | 0.011269899 | 3.27505E−08;
30 | SLE GWAS | 69 | Coronary artery disease || id: ebi-a-GCST005194 | Weighted
mode (NOME) | 59 | −0.075066854 | 0.010651773 | 2.4406E−09; 30 | SLE
GWAS | 69 | Coronary artery disease || id: ebi-a-GCST005194 | Simple mode
(NOME) | 59 | −0.075066854 | 0.012876071 | 2.59489E−07; 30 | SLE
GWAS | 69 | Coronary artery disease || id: ebi-a-GCST005194 | Robust adjusted profile
score (RAPS) | 59 | −0.088080406 | 0.007210768 | 2.58116E−34; 30 | SLE
GWAS | 69 | Coronary artery disease || id: ebi-a-GCST005194 | Raw | 59
| −0.077299804 | 0.007332911 | 4.24019E−15; 30 | SLE GWAS | 69 | Coronary artery disease
|| id: ebi-a-GCST005194 | Outlier-corrected | NA | NA | NA | NA; 31 | SLE
GWAS | 19 | Coronary artery disease || id: ebi-a-GCST005194 | Maximum
likelihood | 16 | 0.066099658 | 0.020536213 | 0.001287786; 31 | SLE
GWAS | 19 | Coronary artery disease || id: ebi-a-GCST005194 | MR
Egger | 16 | 0.062477279 | 0.048923945 | 0.222366352; 31 | SLE GWAS | 19 | Coronary
artery disease || id: ebi-a-GCST005194 | MR Egger
(bootstrap) | 16 | 0.078993324 | 0.048252912 | 0.051; 31 | SLE GWAS | 19 | Coronary
artery disease || id: ebi-a-GCST005194 | Simple
median | 16 | 0.028824899 | 0.041372533 | 0.485980652; 31 | SLE GWAS | 19 | Coronary
artery disease || id: ebi-a-GCST005194 | Weighted
median | 16 | 0.054712796 | 0.026357 | 0.037909168; 31 | SLE GWAS | 19 | Coronary
artery disease || id: ebi-a-GCST005194 | Penalised weighted
median | 16 | 0.060567492 | 0.026136696 | 0.020485465; 31 | SLE GWAS | 19 | Coronary
artery disease || id: ebi-a-GCST005194 | Inverse variance
weighted | 16 | 0.044657009 | 0.029030063 | 0.123974749; 31 | SLE GWAS | 19 | Coronary
artery disease || id: ebi-a-GCST005194 | Inverse variance weighted (multiplicative random
effects) | 16 | 0.044657009 | 0.029030063 | 0.123974749; 31 | SLE GWAS | 19 | Coronary
artery disease || id: ebi-a-GCST005194 | Inverse variance weighted (fixed
effects) | 16 | 0.044657009 | 0.01570681 | 0.004466833; 31 | SLE GWAS | 19 | Coronary
artery disease || id: ebi-a-GCST005194 | Simple
mode | 16 | 0.013152768 | 0.047120412 | 0.783956058; 31 | SLE GWAS | 19 | Coronary
artery disease || id: ebi-a-GCST005194 | Weighted
mode | 16 | 0.056520427 | 0.025217872 | 0.040561054; 31 | SLE GWAS | 19 | Coronary
artery disease || id: ebi-a-GCST005194 | Weighted mode
(NOME) | 16 | 0.065193959 | 0.024776692 | 0.018884926; 31 | SLE GWAS | 19 | Coronary
artery disease || id: ebi-a-GCST005194 | Simple mode
(NOME) | 16 | 0.013152768 | 0.054758861 | 0.81343128; 31 | SLE GWAS | 19 | Coronary
artery disease || id: ebi-a-GCST005194 | Robust adjusted profile score
(RAPS) | 16 | 0.04322277 | 0.03065155 | 0.158500333; 31 | SLE GWAS | 19 | Coronary
artery disease || id: ebi-a-
GCST005194 | Raw | 16 | 0.044657009 | 0.029030063 | 0.144800096; 31 | SLE
GWAS | 19 | Coronary artery disease || id: ebi-a-GCST005194 | Outlier-
corrected | 13 | 0.055517886 | 0.023861518 | 0.038301083; 32 | SLE
GWAS | 26 | Coronary artery disease || id: ebi-a-GCST005194 | Maximum
likelihood | 22 | 0.027328393 | 0.010304049 | 0.007996929; 32 | SLE
GWAS | 26 | Coronary artery disease || id: ebi-a-GCST005194 | MR
Egger | 22 | 0.022640397 | 0.035778067 | 0.534032633; 32 | SLE GWAS | 26 | Coronary
artery disease || id: ebi-a-GCST005194 | MR Egger
(bootstrap) | 22 | 0.00151295 | 0.018779872 | 0.497; 32 | SLE GWAS | 26 | Coronary artery
disease || id: ebi-a-GCST005194 | Simple median | 22
| −0.007811008 | 0.026247922 | 0.766019344; 32 | SLE GWAS | 26 | Coronary artery disease
|| id: ebi-a-GCST005194 | Weighted median | 22
| −0.010192459 | 0.017696982 | 0.56465348; 32 | SLE GWAS | 26 | Coronary artery disease ||
id: ebi-a-GCST005194 | Penalised weighted median | 22
| −0.035941235 | 0.015542271 | 0.020751091; 32 | SLE GWAS | 26 | Coronary artery disease
|| id: ebi-a-GCST005194 | Inverse variance
weighted | 22 | 0.022272316 | 0.018569374 | 0.230368086; 32 | SLE GWAS | 26 | Coronary
artery disease || id: ebi-a-GCST005194 | Inverse variance weighted (multiplicative random
effects) | 22 | 0.022272316 | 0.018569374 | 0.230368086; 32 | SLE GWAS | 26 | Coronary
artery disease || id: ebi-a-GCST005194 | Inverse variance weighted (fixed
effects) | 22 | 0.022272316 | 0.009139682 | 0.014814557; 32 | SLE GWAS | 26 | Coronary
artery disease || id: ebi-a-GCST005194 | Simple mode | 22
| −0.007643401 | 0.032349096 | 0.81550576; 32 | SLE GWAS | 26 | Coronary artery disease ||
id: ebi-a-GCST005194 | Weighted mode | 22 | −0.011507913 | 0.016276492 | 0.487322141;
32 | SLE GWAS | 26 | Coronary artery disease || id: ebi-a-GCST005194 | Weighted mode
(NOME) | 22 | −0.007643401 | 0.017407859 | 0.665089901; 32 | SLE
GWAS | 26 | Coronary artery disease || id: ebi-a-GCST005194 | Simple mode
(NOME) | 22 | −0.007643401 | 0.033300308 | 0.820678263; 32 | SLE
GWAS | 26 | Coronary artery disease || id: ebi-a-GCST005194 | Robust adjusted profile
score (RAPS) | 22 | 0.024501975 | 0.021181743 | 0.24737464; 32 | SLE
GWAS | 26 | Coronary artery disease || id: ebi-a-
GCST005194 | Raw | 22 | 0.022272316 | 0.018569374 | 0.243726523; 32 | SLE
GWAS | 26 | Coronary artery disease || id: ebi-a-GCST005194 | Outlier-
corrected | 19 | 0.017044782 | 0.016960684 | 0.328236271; 33 | SLE
GWAS | 41 | Coronary artery disease || id: ebi-a-GCST005194 | Maximum
likelihood | 36 | 0.028384663 | 0.00547853 | 2.20613E−07; 33 | SLE GWAS | 41 | Coronary
artery disease || id: ebi-a-GCST005194 | MR
Egger | 36 | 0.036937114 | 0.016035763 | 0.027498128; 33 | SLE GWAS | 41 | Coronary
artery disease || id: ebi-a-GCST005194 | MR Egger
(bootstrap) | 36 | 0.034937817 | 0.01102238 | 0.001; 33 | SLE GWAS | 41 | Coronary artery
disease || id: ebi-a-GCST005194 | Simple
median | 36 | 0.027575883 | 0.007916122 | 0.000494886; 33 | SLE GWAS | 41 | Coronary
artery disease || id: ebi-a-GCST005194 | Weighted
median | 36 | 0.028014204 | 0.006992851 | 6.17242E−05; 33 | SLE GWAS | 41 | Coronary
artery disease || id: ebi-a-GCST005194 | Penalised weighted
median | 36 | 0.028001093 | 0.006698669 | 2.91383E−05; 33 | SLE GWAS | 41 | Coronary
artery disease || id: ebi-a-GCST005194 | Inverse variance
weighted | 36 | 0.027780974 | 0.00721341 | 0.000117494; 33 | SLE GWAS | 41 | Coronary
artery disease || id: ebi-a-GCST005194 | Inverse variance weighted (multiplicative random
effects) | 36 | 0.027780974 | 0.00721341 | 0.000117494; 33 | SLE GWAS | 41 | Coronary
artery disease || id: ebi-a-GCST005194 | Inverse variance weighted (fixed
effects) | 36 | 0.027780974 | 0.005315448 | 1.72786E−07; 33 | SLE GWAS | 41 | Coronary
artery disease || id: ebi-a-GCST005194 | Simple
mode | 36 | 0.02711099 | 0.007941733 | 0.001635057; 33 | SLE GWAS | 41 | Coronary
artery disease || id: ebi-a-GCST005194 | Weighted
mode | 36 | 0.02711099 | 0.007183387 | 0.000596858; 33 | SLE GWAS | 41 | Coronary
artery disease || id: ebi-a-GCST005194 | Weighted mode
(NOME) | 36 | 0.02711099 | 0.006762993 | 0.000304416; 33 | SLE GWAS | 41 | Coronary
artery disease || id: ebi-a-GCST005194 | Simple mode
(NOME) | 36 | 0.02711099 | 0.007703075 | 0.001220972; 33 | SLE GWAS | 41 | Coronary
artery disease || id: ebi-a-GCST005194 | Robust adjusted profile score
(RAPS) | 36 | 0.027535731 | 0.005697466 | 1.34506E−06; 33 | SLE GWAS | 41 | Coronary
artery disease || id: ebi-a-
GCST005194 | Raw | 36 | 0.027780974 | 0.00721341 | 0.000478928; 33 | SLE
GWAS | 41 | Coronary artery disease || id: ebi-a-GCST005194 | Outlier-
corrected | 35 | 0.025285984 | 0.006558887 | 0.000489364; 34 | SLE GWAS | 8 | Coronary
artery disease || id: ebi-a-GCST005194 | Maximum
likelihood | 8 | 0.12676842 | 0.017043461 | 1.02259E−13; 34 | SLE GWAS | 8 | Coronary
artery disease || id: ebi-a-GCST005194 | MR
Egger | 8 | 0.130043858 | 0.041422662 | 0.020082249; 34 | SLE GWAS | 8 | Coronary
artery disease || id: ebi-a-GCST005194 | MR Egger
(bootstrap) | 8 | 0.114890967 | 0.03746003 | 0; 34 | SLE GWAS | 8 | Coronary artery
disease || id: ebi-a-GCST005194 | Simple
median | 8 | 0.103119008 | 0.026161924 | 8.09506E−05; 34 | SLE GWAS | 8 | Coronary
artery disease || id: ebi-a-GCST005194 | Weighted
median | 8 | 0.128582851 | 0.021875239 | 4.1523E−09; 34 | SLE GWAS | 8 | Coronary
artery disease || id: ebi-a-GCST005194 | Penalised weighted
median | 8 | 0.129993336 | 0.021763065 | 2.32763E−09; 34 | SLE GWAS | 8 | Coronary
artery disease || id: ebi-a-GCST005194 | Inverse variance
weighted | 8 | 0.117004731 | 0.022564949 | 2.15734E−07; 34 | SLE GWAS | 8 | Coronary
artery disease || id: ebi-a-GCST005194 | Inverse variance weighted (multiplicative random
effects) | 8 | 0.117004731 | 0.022564949 | 2.15734E−07; 34 | SLE GWAS | 8 | Coronary
artery disease || id: ebi-a-GCST005194 | Inverse variance weighted (fixed
effects) | 8 | 0.117004731 | 0.013589278 | 7.30116E−18; 34 | SLE GWAS | 8 | Coronary
artery disease || id: ebi-a-GCST005194 | Simple
mode | 8 | 0.12824114 | 0.028675573 | 0.002893485; 34 | SLE GWAS | 8 | Coronary artery
disease || id: ebi-a-GCST005194 | Weighted
mode | 8 | 0.117504586 | 0.019439454 | 0.000518708; 34 | SLE GWAS | 8 | Coronary
artery disease || id: ebi-a-GCST005194 | Weighted mode
(NOME) | 8 | 0.12363976 | 0.017281338 | 0.000184629; 34 | SLE GWAS | 8 | Coronary
artery disease || id: ebi-a-GCST005194 | Simple mode
(NOME) | 8 | 0.12824114 | 0.024101877 | 0.001098327; 34 | SLE GWAS | 8 | Coronary
artery disease || id: ebi-a-GCST005194 | Robust adjusted profile score
(RAPS) | 8 | 0.130727609 | 0.017871598 | 2.57717E−13; 34 | SLE GWAS | 8 | Coronary
artery disease || id: ebi-a-
GCST005194 | Raw | 8 | 0.117004731 | 0.022564949 | 0.001273515; 34 | SLE
GWAS | 8 | Coronary artery disease || id: ebi-a-GCST005194 | Outlier-
corrected | NA | NA | NA | NA; 35 | SLE GWAS | 163 | Coronary artery disease || id: ebi-a-
GCST005194 | Maximum likelihood | 140 | −0.001334246 | 0.002168176 | 0.538305653;
35 | SLE GWAS | 163 | Coronary artery disease || id: ebi-a-GCST005194 | MR
Egger | 140 | −0.005695371 | 0.005411348 | 0.294415738; 35 | SLE
GWAS | 163 | Coronary artery disease || id: ebi-a-GCST005194 | MR Egger
(bootstrap) | 140 | −0.010810641 | 0.007217628 | 0.064; 35 | SLE GWAS | 163 | Coronary
artery disease || id: ebi-a-GCST005194 | Simple
median | 140 | 0.001790188 | 0.003326911 | 0.590512693; 35 | SLE
GWAS | 163 | Coronary artery disease || id: ebi-a-GCST005194 | Weighted median | 140
| −0.001675603 | 0.003148939 | 0.594645222; 35 | SLE GWAS | 163 | Coronary artery
disease || id: ebi-a-GCST005194 | Penalised weighted median | 140
| −0.001635578 | 0.003178012 | 0.606794617; 35 | SLE GWAS | 163 | Coronary artery
disease || id: ebi-a-GCST005194 | Inverse variance weighted | 140
| −0.001317224 | 0.002797366 | 0.637726315; 35 | SLE GWAS | 163 | Coronary artery
disease || id: ebi-a-GCST005194 | Inverse variance weighted (multiplicative random
effects) | 140 | −0.001317224 | 0.002797366 | 0.637726315; 35 | SLE
GWAS | 163 | Coronary artery disease || id: ebi-a-GCST005194 | Inverse variance weighted
(fixed effects) | 140 | −0.001317224 | 0.002135363 | 0.537325841; 35 | SLE
GWAS | 163 | Coronary artery disease || id: ebi-a-GCST005194 | Simple mode | 140
| −0.00098413 | 0.006873592 | 0.886358977; 35 | SLE GWAS | 163 | Coronary artery disease
| id: ebi-a-GCST005194 | Weighted mode | 140 | −0.00098413 | 0.005400266 | 0.85566207;
35 | SLE GWAS | 163 | Coronary artery disease || id: ebi-a-GCST005194 | Weighted mode
(NOME) | 140 | −0.00098413 | 0.004019283 | 0.806932096; 35 | SLE
GWAS | 163 | Coronary artery disease || id: ebi-a-GCST005194 | Simple mode
(NOME) | 140 | −0.00098413 | 0.00584936 | 0.866634564; 35 | SLE
GWAS | 163 | 140 | −0.00123334 | 0.002667859 | 0.643869144; 35 | SLE
score (RAPS) | Coronary artery disease || id: ebi-a-GCST005194 | Robust adjusted profile
GWAS | 163 | Coronary artery disease || id: ebi-a-GCST005194 | Raw | 140
| −0.001317224 | 0.002797366 | 0.638464437; 35 | SLE GWAS | 163 | Coronary artery
disease || id: ebi-a-GCST005194 | Outlier-corrected | 138
| −0.001146076 | 0.002398482 | 0.633530052; 36 | SLE GWAS | 59 | Coronary artery disease
|| id: ebi-a-GCST005194 | Maximum
likelihood | 46 | 0.013479591 | 0.006676407 | 0.043488389; 36 | SLE
GWAS | 59 | Coronary artery disease || id: ebi-a-GCST005194 | MR
Egger | 46 | 0.007948478 | 0.020999107 | 0.706868274; 36 | SLE GWAS | 59 | Coronary
artery disease || id: ebi-a-GCST005194 | MR Egger | (bootstrap) | 46
| −0.00171898 | 0.014337016 | 0.462; 36 | SLE GWAS | 59 | Coronary artery disease || id: ebi-
a-GCST005194 | Simple median | 46 | 0.025659682 | 0.009590494 | 0.007461053; 36 | SLE
GWAS | 59 | Coronary artery disease || id: ebi-a-GCST005194 | Weighted
median | 46 | 0.02104092 | 0.00883861 | 0.017285928; 36 | SLE GWAS | 59 | Coronary
artery disease || id: ebi-a-GCST005194 | Penalised weighted
median | 46 | 0.02104092 | 0.008925423 | 0.018402702; 36 | SLE GWAS | 59 | Coronary
artery disease || id: ebi-a-GCST005194 | Inverse variance
weighted | 46 | 0.013128296 | 0.006559969 | 0.045362856; 36 | SLE GWAS | 59 | Coronary
artery disease || id: ebi-a-GCST005194 | Inverse variance weighted (multiplicative random
effects) | 46 | 0.013128296 | 0.0051168 | 0.010296072; 36 | SLE GWAS | 59 | Coronary
artery disease || id: ebi-a-GCST005194 | Inverse variance weighted (fixed
effects) | 46 | 0.013128296 | 0.006559969 | 0.045362856; 36 | SLE GWAS | 59 | Coronary
artery disease || id: ebi-a-GCST005194 | Simple
mode | 46 | 0.031094319 | 0.015645893 | 0.052987058; 36 | SLE GWAS | 59 | Coronary
artery disease || id: ebi-a-GCST005194 | Weighted
mode | 46 | 0.027957759 | 0.013708691 | 0.047306133; 36 | SLE GWAS | 59 | Coronary
artery disease || id: ebi-a-GCST005194 | Weighted mode
(NOME) | 46 | 0.031094319 | 0.014789185 | 0.041135481; 36 | SLE GWAS | 59 | Coronary
artery disease || id: ebi-a-GCST005194 | Simple mode
(NOME) | 46 | 0.031094319 | 0.016187139 | 0.061089986; 36 | SLE GWAS | 59 | Coronary
artery disease || id: ebi-a-GCST005194 | Robust adjusted profile score
(RAPS) | 46 | 0.013020709 | 0.006981732 | 0.062185827; 36 | SLE GWAS | 59 | Coronary
artery disease || id: ebi-a-
GCST005194 | Raw | 46 | 0.013128296 | 0.0051168 | 0.013697813; 36 | SLE
GWAS | 59 | Coronary artery disease || id: ebi-a-GCST005194 | Outlier-
corrected | NA | NA | NA | NA; 37 | SLE GWAS | 2 | Coronary artery disease || id: ebi-a-
GCST005194 | Maximum likelihood | 2 | 0.577575085 | 0.524644651 | 0.270945336;
37 | SLE GWAS | 2 | Coronary artery disease || id: ebi-a-GCST005194 | Inverse variance
weighted | 2 | 0.275873213 | 0.320243194 | 0.388990741; 37 | SLE GWAS | 2 | Coronary
artery disease || id: ebi-a-GCST005194 | Inverse variance weighted (multiplicative random
effects) | 2 | 0.275873213 | 0.320243194 | 0.388990741; 37 | SLE GWAS | 2 | Coronary
artery disease || id: ebi-a-GCST005194 | Inverse variance weighted (fixed
effects) | 2 | 0.275873213 | 0.132951808 | 0.037987782; 37 | SLE GWAS | 2 | Coronary
artery disease || id: ebi-a-GCST005194 | Robust adjusted profile score
(RAPS) | 2 | 0.577653475 | 0.580385456 | 0.319593871; 38 | SLE GWAS | 15 | Coronary
artery disease || id: ebi-a-GCST005194 | Maximum
likelihood | 14 | 0.203958479 | 0.047607699 | 1.8344E−05; 38 | SLE GWAS | 15 | Coronary
artery disease || id: ebi-a-GCST005194 | MR
Egger | 14 | 0.322611272 | 0.130928911 | 0.029818303; 38 | SLE GWAS | 15 | Coronary
artery disease || id: ebi-a-GCST005194 | MR Egger
(bootstrap) | 14 | 0.263021786 | 0.065360637 | 0.001; 38 | SLE GWAS | 15 | Coronary
artery disease || id: ebi-a-GCST005194 | Simple
median | 14 | 0.008656195 | 0.043218959 | 0.841256133; 38 | SLE GWAS | 15 | Coronary
artery disease || id: ebi-a-GCST005194 | Weighted
median | 14 | 0.043412359 | 0.036266177 | 0.231287795; 38 | SLE GWAS | 15 | Coronary
artery disease || id: ebi-a-GCST005194 | Penalised weighted
median | 14 | 0.041346164 | 0.035633138 | 0.245914868; 38 | SLE GWAS | 15 | Coronary
artery disease || id: ebi-a-GCST005194 | Inverse variance
weighted | 14 | 0.090973393 | 0.067606583 | 0.178422123; 38 | SLE GWAS | 15 | Coronary
artery disease || id: ebi-a-GCST005194 | Inverse variance weighted (multiplicative random
effects) | 14 | 0.090973393 | 0.067606583 | 0.178422123; 38 | SLE GWAS | 15 | Coronary
artery disease || id: ebi-a-GCST005194 | Inverse variance weighted (fixed
effects) | 14 | 0.090973393 | 0.020594766 | 9.99384E−06; 38 | SLE GWAS | 15 | Coronary
artery disease || id: ebi-a-GCST005194 | Simple
mode | 14 | 0.037142579 | 0.040198605 | 0.372331314; 38 | SLE GWAS | 15 | Coronary
artery disease || id: ebi-a-GCST005194 | Weighted
mode | 14 | 0.04225039 | 0.036740837 | 0.270877327; 38 | SLE GWAS | 15 | Coronary
artery disease || id: ebi-a-GCST005194 | Weighted mode
(NOME) | 14 | 0.04225039 | 0.048581245 | 0.400245626; 38 | SLE GWAS | 15 | Coronary
artery disease || id: ebi-a-GCST005194 | Simple mode
(NOME) | 14 | 0.037142579 | 0.038427183 | 0.351397205; 38 | SLE GWAS | 15 | Coronary
artery disease || id: ebi-a-GCST005194 | Robust adjusted profile score
(RAPS) | 14 | 0.033069533 | 0.079354388 | 0.676874258; 38 | SLE GWAS | 15 | Coronary
artery disease || id: ebi-a-
GCST005194 | Raw | 14 | 0.090973393 | 0.067606583 | 0.201422872; 38 | SLE
GWAS | 15 | Coronary artery disease || id: ebi-a-GCST005194 | Outlier-
corrected | 10 | 0.033729111 | 0.015264257 | 0.054473739; 39 | SLE
GWAS | 12 | Coronary artery disease || id: ebi-a-GCST005194 | Maximum
likelihood | 10 | 0.166578177 | 0.029277673 | 1.27339E−08; 39 | SLE
GWAS | 12 | Coronary artery disease || id: ebi-a-GCST005194 | MR Egger | 10
| −0.038176858 | 0.077996444 | 0.637647649; 39 | SLE GWAS | 12 | Coronary artery disease
|| id: ebi-a-GCST005194 | MR Egger | (bootstrap) | 10 | 0.207489721 | 0.083369213 | 0.004;
39 | SLE GWAS | 12 | Coronary artery disease || id: ebi-a-GCST005194 | Simple
median | 10 | 0.101965017 | 0.04748218 | 0.031758462; 39 | SLE GWAS | 12 | Coronary
artery disease || id: ebi-a-GCST005194 | Weighted median | 10
| −0.006075473 | 0.028042407 | 0.828478733; 39 | SLE GWAS | 12 | Coronary artery disease
|| id: ebi-a-GCST005194 | Penalised weighted median | 10
| −0.007056267 | 0.027637281 | 0.798478284; 39 | SLE GWAS | 12 | Coronary artery disease
|| id: ebi-a-GCST005194 | Inverse variance
weighted | 10 | 0.102379609 | 0.056630215 | 0.070627996; 39 | SLE GWAS | 12 | Coronary
artery disease || id: ebi-a-GCST005194 | Inverse variance weighted (multiplicative random
effects) | 10 | 0.102379609 | 0.056630215 | 0.070627996; 39 | SLE GWAS | 12 | Coronary
artery disease || id: ebi-a-GCST005194 | Inverse variance weighted (fixed
effects) | 10 | 0.102379609 | 0.017382154 | 3.86368E−09; 39 | SLE GWAS | 12 | Coronary
artery disease || id: ebi-a-GCST005194 | Simple
mode | 10 | 0.063021937 | 0.0409178 | 0.157896221; 39 | SLE GWAS | 12 | Coronary
artery disease || id: ebi-a-GCST005194 | Weighted
mode | 10 | 0.015436806 | 0.025666152 | 0.56238409; 39 | SLE GWAS | 12 | Coronary
artery disease || id: ebi-a-GCST005194 | Weighted mode
(NOME) | 10 | 0.023367661 | 0.025960262 | 0.391498769; 39 | SLE GWAS | 12 | Coronary
artery disease || id: ebi-a-GCST005194 | Simple mode
(NOME) | 10 | 0.063021937 | 0.040526321 | 0.154347619; 39 | SLE GWAS | 12 | Coronary
artery disease || id: ebi-a-GCST005194 | Robust adjusted profile score
(RAPS) | 10 | 0.020735875 | 0.04244399 | 0.625162591; 39 | SLE GWAS | 12 | Coronary
artery disease || id: ebi-a-
GCST005194 | Raw | 10 | 0.102379609 | 0.056630215 | 0.104086832; 39 | SLE
GWAS | 12 | Coronary artery disease || id: ebi-a-GCST005194 | Outlier-
corrected | 7 | 0.056869251 | 0.029183814 | 0.099243537; 40 | SLE GWAS | 4 | Coronary
artery disease || id: ebi-a-GCST005194 | Maximum likelihood | 4
| −0.230727841 | 0.101292677 | 0.022736506; 40 | SLE GWAS | 4 | Coronary artery disease ||
id: ebi-a-GCST005194 | MR Egger | 4 | −0.279900521 | 0.1968023 | 0.2908946; 40 | SLE
GWAS | 4 | Coronary artery disease || id: ebi-a-GCST005194 | MR Egger | (bootstrap) | 4
| −0.156124192 | 0.28983918 | 0.223; 40 | SLE GWAS | 4 | Coronary artery disease || id: ebi-a-
GCST005194 | Simple median | 4 | −0.155259034 | 0.25685192 | 0.545531845; 40 | SLE
GWAS | 4 | Coronary artery disease || id: ebi-a-GCST005194 | Weighted median | 4
| −0.145309309 | 0.624446084 | 0.815993492; 40 | SLE GWAS | 4 | Coronary artery disease ||
id: ebi-a-GCST005194 | Penalised weighted median | 4
| −0.145309309 | 0.197494174 | 0.461873699; 40 | SLE GWAS | 4 | Coronary artery disease ||
id: ebi-a-GCST005194 | Inverse variance weighted | 4
| −0.198422102 | 0.065230605 | 0.002351244; 40 | SLE GWAS | 4 | Coronary artery disease ||
id: ebi-a-GCST005194 | Inverse variance weighted (multiplicative random effects) | 4
| −0.198422102 | 0.065230605 | 0.002351244; 40 | SLE GWAS | 4 | Coronary artery disease ||
id: ebi-a-GCST005194 | Inverse variance weighted (fixed effects) | 4
| −0.198422102 | 0.061714945 | 0.001303816; 40 | SLE GWAS | 4 | Coronary artery disease ||
id: ebi-a-GCST005194 | Simple mode | 4 | −0.247623838 | 0.148424689 | 0.193838714;
40 | SLE GWAS | 4 | Coronary artery disease || id: ebi-a-GCST005194 | Weighted
mode | 4 | −0.121103903 | 0.114084014 | 0.366333106; 40 | SLE GWAS | 4 | Coronary
artery disease || id: ebi-a-GCST005194 | Weighted mode (NOME) | 4
| −0.245479433 | 0.084763783 | 0.062703813; 40 | SLE GWAS | 4 | Coronary artery disease ||
id: ebi-a-GCST005194 | Simple mode (NOME) | 4
| −0.247623838 | 0.106542866 | 0.102692363; 40 | SLE GWAS | 4 | Coronary artery disease ||
id: ebi-a-GCST005194 | Robust adjusted profile score (RAPS) | 4
| −0.230716094 | 0.110699366 | 0.037144874; 40 | SLE GWAS | 4 | Coronary artery disease ||
id: ebi-a-GCST005194 | Raw | 4 | −0.198422102 | 0.065230605 | 0.055785453; 40 | SLE
GWAS | 4 | Coronary artery disease || id: ebi-a-GCST005194 | Outlier-
corrected | NA | NA | NA | NA; 41 | SLE GWAS | 19 | Coronary artery disease || id: ebi-a-
GCST005194 | Maximum likelihood | 17 | 0.086533541 | 0.032018317 | 0.006879407;
41 | SLE GWAS | 19 | Coronary artery disease || id: ebi-a-GCST005194 | MR Egger | 17
| −0.104713982 | 0.067205442 | 0.140050414; 41 | SLE GWAS | 19 | Coronary artery disease
|| id: ebi-a-GCST005194 | MR Egger | (bootstrap) | 17 | −0.013706626 | 0.07104363 | 0.407;
41 | SLE GWAS | 19 | Coronary artery disease || id: ebi-a-GCST005194 | Simple
median | 17 | 0.072079607 | 0.041793778 | 0.084590706; 41 | SLE GWAS | 19 | Coronary
artery disease || id: ebi-a-GCST005194 | Weighted median | 17
| −0.006408996 | 0.033364217 | 0.847670221; 41 | SLE GWAS | 19 | Coronary artery disease
|| id: ebi-a-GCST005194 | Penalised weighted median | 17
| −0.006559855 | 0.032086579 | 0.838007843; 41 | SLE GWAS | 19 | Coronary artery disease
|| id: ebi-a-GCST005194 | Inverse variance
weighted | 17 | 0.053283674 | 0.032610667 | 0.10227273; 41 | SLE GWAS | 19 | Coronary
artery disease || id: ebi-a-GCST005194 | Inverse variance weighted (multiplicative random
effects) | 17 | 0.053283674 | 0.032610667 | 0.10227273; 41 | SLE GWAS | 19 | Coronary
artery disease || id: ebi-a-GCST005194 | Inverse variance weighted (fixed
effects) | 17 | 0.053283674 | 0.022732466 | 0.019080928; 41 | SLE GWAS | 19 | Coronary
artery disease || id: ebi-a-GCST005194 | Simple
mode | 17 | 0.0126533 | 0.036569362 | 0.733844444; 41 | SLE GWAS | 19 | Coronary
artery disease || id: ebi-a-GCST005194 | Weighted
mode | 17 | 0.001679421 | 0.030807251 | 0.957200681; 41 | SLE GWAS | 19 | Coronary
artery disease || id: ebi-a-GCST005194 | Weighted mode
(NOME) | 17 | 0.003508401 | 0.032775029 | 0.916084066; 41 | SLE GWAS | 19 | Coronary
artery disease || id: ebi-a-GCST005194 | Simple mode
(NOME) | 17 | 0.0126533 | 0.036567646 | 0.733832473; 41 | SLE GWAS | 19 | Coronary
artery disease || id: ebi-a-GCST005194 | Robust adjusted profile score
(RAPS) | 17 | 0.048472743 | 0.037551214 | 0.196757926; 41 | SLE GWAS | 19 | Coronary
artery disease || id: ebi-a-
GCST005194 | Raw | 17 | 0.053283674 | 0.032610667 | 0.121792381; 41 | SLE
GWAS | 19 | Coronary artery disease || id: ebi-a-GCST005194 | Outlier-
corrected | 16 | 0.030755397 | 0.023928429 | 0.21817603; 42 | SLE GWAS | 23 | Coronary
artery disease || id: ebi-a-GCST005194 | Maximum
likelihood | 20 | 0.117029062 | 0.030615232 | 0.000132065; 42 | SLE
GWAS | 23 | Coronary artery disease || id: ebi-a-GCST005194 | MR
Egger | 20 | 0.022733871 | 0.066021616 | 0.734580965; 42 | SLE GWAS | 23 | Coronary
artery disease || id: ebi-a-GCST005194 | MR Egger
(bootstrap) | 20 | 0.068117796 | 0.048225445 | 0.078; 42 | SLE GWAS | 23 | Coronary
artery disease || id: ebi-a-GCST005194 | Simple
median | 20 | 0.038888276 | 0.044962083 | 0.387086725; 42 | SLE GWAS | 23 | Coronary
artery disease || id: ebi-a-GCST005194 | Weighted
median | 20 | 0.021807157 | 0.033801872 | 0.518832311; 42 | SLE GWAS | 23 | Coronary
artery disease || id: ebi-a-GCST005194 | Penalised weighted
median | 20 | 0.022931879 | 0.033869764 | 0.498367565; 42 | SLE GWAS | 23 | Coronary
artery disease || id: ebi-a-GCST005194 | Inverse variance
weighted | 20 | 0.052672697 | 0.036493483 | 0.148923221; 42 | SLE GWAS | 23 | Coronary
artery disease || id: ebi-a-GCST005194 | Inverse variance weighted (multiplicative random
effects) | 20 | 0.052672697 | 0.036493483 | 0.148923221; 42 | SLE GWAS | 23 | Coronary
artery disease || id: ebi-a-GCST005194 | Inverse variance weighted (fixed
effects) | 20 | 0.052672697 | 0.018238977 | 0.003877992; 42 | SLE GWAS | 23 | Coronary
artery disease || id: ebi-a-GCST005194 | Simple
mode | 20 | 0.074246683 | 0.056165968 | 0.201891725; 42 | SLE GWAS | 23 | Coronary
artery disease || id: ebi-a-GCST005194 | Weighted
mode | 20 | 0.012109927 | 0.028390891 | 0.67450627; 42 | SLE GWAS | 23 | Coronary
artery disease || id: ebi-a-GCST005194 | Weighted mode
(NOME) | 20 | 0.027644116 | 0.038109597 | 0.477056213; 42 | SLE GWAS | 23 | Coronary
artery disease || id: ebi-a-GCST005194 | Simple mode
(NOME) | 20 | 0.074246683 | 0.067587401 | 0.28569995; 42 | SLE GWAS | 23 | Coronary
artery disease || id: ebi-a-GCST005194 | Robust adjusted profile score
(RAPS) | 20 | 0.049347956 | 0.047880834 | 0.302709147; 42 | SLE GWAS | 23 | Coronary
artery disease || id: ebi-a-
GCST005194 | Raw | 20 | 0.052672697 | 0.036493483 | 0.165209178; 42 | SLE
GWAS | 23 | Coronary artery disease || id: ebi-a-GCST005194 | Outlier-
corrected | 16 | 0.047292947 | 0.032357811 | 0.164493532; 43 | SLE
GWAS | 27 | Coronary artery disease || id: ebi-a-GCST005194 | Maximum
likelihood | 18 | 0.03171484 | 0.013608827 | 0.019781796; 43 | SLE GWAS | 27 | Coronary
artery disease || id: ebi-a-GCST005194 | MR
Egger | 18 | 0.069815148 | 0.037441261 | 0.080679762; 43 | SLE GWAS | 27 | Coronary
artery disease || id: ebi-a-GCST005194 | MR Egger
(bootstrap) | 18 | 0.061505672 | 0.024237359 | 0.005; 43 | SLE GWAS | 27 | Coronary
artery disease || id: ebi-a-GCST005194 | Simple
median | 18 | 0.017752998 | 0.03306224 | 0.591297378; 43 | SLE GWAS | 27 | Coronary
artery disease || id: ebi-a-GCST005194 | Weighted
median | 18 | 0.054594227 | 0.018820866 | 0.00372296; 43 | SLE GWAS | 27 | Coronary
artery disease || id: ebi-a-GCST005194 | Penalised weighted
median | 18 | 0.064589872 | 0.018197921 | 0.000386257; 43 | SLE GWAS | 27 | Coronary
artery disease || id: ebi-a-GCST005194 | Inverse variance
weighted | 18 | 0.02845562 | 0.021165223 | 0.178802375; 43 | SLE GWAS | 27 | Coronary
artery disease || id: ebi-a-GCST005194 | Inverse variance weighted (multiplicative random
effects) | 18 | 0.02845562 | 0.021165223 | 0.178802375; 43 | SLE GWAS | 27 | Coronary
artery disease || id: ebi-a-GCST005194 | Inverse variance weighted (fixed
effects) | 18 | 0.02845562 | 0.01201946 | 0.017910542; 43 | SLE GWAS | 27 | Coronary
artery disease || id: ebi-a-GCST005194 | Simple
mode | 18 | 0.04013914 | 0.031198075 | 0.215482593; 43 | SLE GWAS | 27 | Coronary
artery disease || id: ebi-a-GCST005194 | Weighted
mode | 18 | 0.052383992 | 0.014911071 | 0.002667234; 43 | SLE GWAS | 27 | Coronary
artery disease || id: ebi-a-GCST005194 | Weighted mode
(NOME) | 18 | 0.052383992 | 0.014913024 | 0.002669897; 43 | SLE GWAS | 27 | Coronary
artery disease || id: ebi-a-GCST005194 | Simple mode
(NOME) | 18 | 0.04013914 | 0.031573258 | 0.220740977; 43 | SLE GWAS | 27 | Coronary
artery disease || id: ebi-a-GCST005194 | Robust adjusted profile score
(RAPS) | 18 | 0.02917555 | 0.024857217 | 0.240504991; 43 | SLE GWAS | 27 | Coronary
artery disease || id: ebi-a-
GCST005194 | Raw | 18 | 0.02845562 | 0.021165223 | 0.196468699; 43 | SLE
GWAS | 27 | Coronary artery disease || id: ebi-a-GCST005194 | Outlier-
corrected | 17 | 0.033601764 | 0.019818671 | 0.109356854; 44 | SLE
GWAS | 36 | Coronary artery disease || id: ebi-a-GCST005194 | Maximum
likelihood | 35 | 0.024281147 | 0.004580508 | 1.15188E−07; 44 | SLE
GWAS | 36 | Coronary artery disease || id: ebi-a-GCST005194 | MR
Egger | 35 | 0.026027966 | 0.013502534 | 0.06254409; 44 | SLE GWAS | 36 | Coronary
artery disease || id: ebi-a-GCST005194 | MR Egger
(bootstrap) | 35 | 0.046548955 | 0.013633832 | 0; 44 | SLE GWAS | 36 | Coronary artery
disease || id: ebi-a-GCST005194 | Simple
median | 35 | 0.027140881 | 0.006703736 | 5.15205E−05; 44 | SLE GWAS | 36 | Coronary
artery disease || id: ebi-a-GCST005194 | Weighted
median | 35 | 0.027147111 | 0.006031158 | 6.75887E−06; 44 | SLE GWAS | 36 | Coronary
artery disease || id: ebi-a-GCST005194 | Penalised weighted
median | 35 | 0.027146355 | 0.00627865 | 1.53505E−05; 44 | SLE GWAS | 36 | Coronary
artery disease || id: ebi-a-GCST005194 | Inverse variance
weighted | 35 | 0.024230323 | 0.005117314 | 2.19088E−06; 44 | SLE GWAS | 36 | Coronary
artery disease || id: ebi-a-GCST005194 | Inverse variance weighted (multiplicative random
effects) | 35 | 0.024230323 | 0.005117314 | 2.19088E−06; 44 | SLE GWAS | 36 | Coronary
artery disease || id: ebi-a-GCST005194 | Inverse variance weighted (fixed
effects) | 35 | 0.024230323 | 0.004514433 | 7.99232E−08; 44 | SLE GWAS | 36 | Coronary
artery disease || id: ebi-a-GCST005194 | Simple
mode | 35 | 0.027842213 | 0.009751833 | 0.007281579; 44 | SLE GWAS | 36 | Coronary
artery disease || id: ebi-a-GCST005194 | Weighted
mode | 35 | 0.028770296 | 0.008776184 | 0.002413263; 44 | SLE GWAS | 36 | Coronary
artery disease || id: ebi-a-GCST005194 | Weighted mode
(NOME) | 35 | 0.028770296 | 0.009281919 | 0.00387677; 44 | SLE GWAS | 36 | Coronary
artery disease || id: ebi-a-GCST005194 | Simple mode
(NOME) | 35 | 0.027842213 | 0.009416674 | 0.005619071; 44 | SLE GWAS | 36 | Coronary
artery disease || id: ebi-a-GCST005194 | Robust adjusted profile score
(RAPS) | 35 | 0.024193262 | 0.004743792 | 3.39682E−07; 44 | SLE GWAS | 36 | Coronary
artery disease || id: ebi-a-GCST005194 | Raw | 35 | 0.024230323 | 0.005117314 | 3.77952E−05;
44 | SLE GWAS | 36 | Coronary artery disease || id: ebi-a-GCST005194 | Outlier-
corrected | NA | NA | NA | NA; 45 | SLE GWAS | 77 | Coronary artery disease || id: ebi-a-
GCST005194 | Maximum likelihood | 71 | −0.039960862 | 0.004654206 | 9.00752E−18;
45 | SLE GWAS | 77 | Coronary artery disease || id: ebi-a-GCST005194 | MR Egger | 71
| −0.050575161 | 0.019789591 | 0.012806057; 45 | SLE GWAS | 77 | Coronary artery disease
|| id: ebi-a-GCST005194 | MR Egger | (bootstrap) | 71 −0.036914678 | 0.009212316 | 0;
45 | SLE GWAS | 77 | Coronary artery disease || id: ebi-a-GCST005194 | Simple
median | 71 | −0.017779982 | 0.007385634 | 0.016067714; 45 | SLE GWAS | 77 | Coronary
artery disease || id: ebi-a-GCST005194 | Weighted median | 71
| −0.026313787 | 0.006329426 | 3.21928E−05; 45 | SLE GWAS | 77 | Coronary artery disease
|| id: ebi-a-GCST005194 | Penalised weighted median | 71
| −0.026313787 | 0.006720196 | 9.01692E−05; 45 | SLE GWAS | 77 | Coronary artery disease
|| id: ebi-a-GCST005194 | Inverse variance weighted | 71
| −0.038603069 | 0.004836351 | 1.44146E−15; 45 | SLE GWAS | 77 | Coronary artery disease
|| id: ebi-a-GCST005194 | Inverse variance weighted (multiplicative random effects) | 71
| −0.038603069 | 0.004836351 | 1.44146E−15; 45 | SLE GWAS | 77 | Coronary artery disease
|| id: ebi-a-GCST005194 | Inverse variance weighted (fixed effects) | 71
| −0.038603069 | 0.004417093 | 2.34198E−18; 45 | SLE GWAS | 77 | Coronary artery disease
|| id: ebi-a-GCST005194 | Simple mode | 71 | −0.015924216 | 0.016271691 | 0.33112542;
45 | SLE GWAS | 77 | Coronary artery disease || id: ebi-a-GCST005194 | Weighted
mode | 71 | −0.017042623 | 0.014651384 | 0.248694619; 45 | SLE GWAS | 77 | Coronary
artery disease || id: ebi-a-GCST005194 | Weighted mode (NOME) | 71
| −0.018161029 | 0.015957736 | 0.258971282; 45 | SLE GWAS | 77 | Coronary artery disease
|| id: ebi-a-GCST005194 | Simple mode (NOME) | 71
| −0.015924216 | 0.015904096 | 0.320147367; 45 | SLE GWAS | 77 | Coronary artery disease
|| id: ebi-a-GCST005194 | Robust adjusted profile score (RAPS) | 71
| −0.040324756 | 0.004997781 | 7.11484E−16; 45 | SLE GWAS | 77 | Coronary artery disease
|| id: ebi-a-GCST005194 | Raw | 71 | −0.038603069 | 0.004836351 | 1.97291E−11; 45 | SLE
GWAS | 77 | Coronary artery disease || id: ebi-a-GCST005194 | Outlier-
corrected | NA | NA | NA | NA; 46 | SLE GWAS | 8 | Coronary artery disease || id: ebi-a-
GCST005194 | Maximum likelihood | 6 | 0.208528109 | 0.054730536 | 0.000138918;
46 | SLE GWAS | 8 | Coronary artery disease || id: ebi-a-GCST005194 | MR
Egger | 6 | 0.371972102 | 0.412124926 | 0.417790824; 46 | SLE GWAS | 8 | Coronary
artery disease || id: ebi-a-GCST005194 | MR Egger
(bootstrap) | 6 | 0.073512703 | 0.08725636 | 0.192; 46 | SLE GWAS | 8 | Coronary artery
disease || id: ebi-a-GCST005194 | Simple
median | 6 | 0.041087383 | 0.07541302 | 0.585869467; 46 | SLE GWAS | 8 | Coronary
artery disease || id: ebi-a-GCST005194 | Weighted
median | 6 | 0.09293994 | 0.055804025 | 0.095818984; 46 | SLE GWAS | 8 | Coronary
artery disease || id: ebi-a-GCST005194 | Penalised weighted
median | 6 | 0.142536834 | 0.051451678 | 0.005600385; 46 | SLE GWAS | 8 | Coronary
artery disease || id: ebi-a-GCST005194 | Inverse variance
weighted | 6 | 0.103388074 | 0.070190974 | 0.140763448; 46 | SLE GWAS | 8 | Coronary
artery disease || id: ebi-a-GCST005194 | Inverse variance weighted (multiplicative random
effects) | 6 | 0.103388074 | 0.070190974 | 0.140763448; 46 | SLE GWAS | 8 | Coronary
artery disease || id: ebi-a-GCST005194 | Inverse variance weighted (fixed
effects) | 6 | 0.103388074 | 0.026696289 | 0.000107614; 46 | SLE GWAS | 8 | Coronary
artery disease || id: ebi-a-GCST005194 | Simple mode | 6
| −0.026269636 | 0.102749084 | 0.808391955; 46 | SLE GWAS | 8 | Coronary artery disease ||
id: ebi-a-GCST005194 | Weighted mode | 6 | 0.110288917 | 0.05238861 | 0.089162154;
46 | SLE GWAS | 8 | Coronary artery disease || id: ebi-a-GCST005194 | Weighted mode
(NOME) | 6 | 0.175416843 | 0.051520516 | 0.019151116; 46 | SLE GWAS | 8 | Coronary
artery disease || id: ebi-a-GCST005194 | Simple mode (NOME) | 6
| −0.026269636 | 0.090529698 | 0.783330318; 46 | SLE GWAS | 8 | Coronary artery disease ||
id: ebi-a-GCST005194 | Robust adjusted profile score
(RAPS) | 6 | 0.136783652 | 0.083303761 | 0.1005928; 46 | SLE GWAS | 8 | Coronary
artery disease || id: ebi-a-
GCST005194 | Raw | 6 | 0.103388074 | 0.070190974 | 0.200753157; 46 | SLE
GWAS | 8 | Coronary artery disease || id: ebi-a-GCST005194 | Outlier-
corrected | 3 | 0.081352704 | 0.029393881 | 0.109516326; 47 | SLE GWAS | 16 | Coronary
artery disease || id: ebi-a-GCST005194 | Maximum likelihood | 14
| −0.029789667 | 0.0214566 | 0.16502488; 47 | SLE GWAS | 16 | Coronary artery disease ||
id: ebi-a-GCST005194 | MR Egger | 14 | 0.021098354 | 0.032420034 | 0.527452405;
47 | SLE GWAS | 16 | Coronary artery disease || id: ebi-a-GCST005194 | MR Egger
(bootstrap) | 14 | 0.001502648 | 0.061688261 | 0.496; 47 | SLE GWAS | 16 | Coronary
artery disease || id: ebi-a-GCST005194 | Simple median | 14
| −0.036507176 | 0.036266097 | 0.3141042; 47 | SLE GWAS | 16 | Coronary artery disease ||
id: ebi-a-GCST005194 | Weighted median | 14 | −0.011306348 | 0.02538648 | 0.656053208;
47 | SLE GWAS | 16 | Coronary artery disease || id: ebi-a-GCST005194 | Penalised
weighted median | 14 | −0.011306348 | 0.024927824 | 0.650143129; 47 | SLE
GWAS | 16 | Coronary artery disease || id: ebi-a-GCST005194 | Inverse variance
weighted | 14 | −0.026354801 | 0.022961924 | 0.251067237; 47 | SLE
GWAS | 16 | Coronary artery disease || id: ebi-a-GCST005194 | Inverse variance weighted
(multiplicative random effects) | 14 | −0.026354801 | 0.022961924 | 0.251067237; 47 | SLE
GWAS | 16 | Coronary artery disease || id: ebi-a-GCST005194 | Inverse variance weighted
(fixed effects) | 14 | −0.026354801 | 0.019518301 | 0.176932276; 47 | SLE
GWAS | 16 | Coronary artery disease || id: ebi-a-GCST005194 | Simple mode | 14
| −0.017661557 | 0.033711959 | 0.609167182; 47 | SLE GWAS | 16 | Coronary artery disease
|| id: ebi-a-GCST005194 | Weighted mode | 14
| −0.015067511 | 0.021921855 | 0.503954137; 47 | SLE GWAS | 16 | Coronary artery disease
|| id: ebi-a-GCST005194 | Weighted mode (NOME) | 14
| −0.015067511 | 0.023408088 | 0.530969893; 47 | SLE GWAS | 16 | Coronary artery disease
|| id: ebi-a-GCST005194 | Simple mode (NOME) | 14
| −0.017661557 | 0.031665066 | 0.586484213; 47 | SLE GWAS | 16 | Coronary artery disease
|| id: ebi-a-GCST005194 | Robust adjusted profile score (RAPS) | 14
| −0.026715285 | 0.026355442 | 0.310748143; 47 | SLE GWAS | 16 | Coronary artery disease
|| id: ebi-a-GCST005194 | Raw | 14 | −0.026354801 | 0.022961924 | 0.271750896; 47 | SLE
GWAS | 16 | Coronary artery disease || id: ebi-a-GCST005194 | Outlier-
corrected | NA | NA | NA | NA; 48 | SLE GWAS | 3 | Coronary artery disease || id: ebi-a-
GCST005194 | Maximum likelihood | 3 | 0.875124916 | 0.612883219 | 0.153325825;
48 | SLE GWAS | 3 | Coronary artery disease || id: ebi-a-GCST005194 | MR
Egger | 3 | 0.886074395 | 0.583047221 | 0.37050422; 48 | SLE GWAS | 3 | Coronary artery
disease || id: ebi-a-GCST005194 | MR Egger | (bootstrap) | 3
| −0.276778033 | 23.63391145 | 0.833; 48 | SLE GWAS | 3 | Coronary artery disease || id: ebi-
a-GCST005194 | Simple median | 3 | 0.651308882 | 0.921999 | 0.479933495; 48 | SLE
GWAS | 3 | Coronary artery disease || id: ebi-a-GCST005194 | Weighted
median | 3 | 0.380340445 | 16.79481637 | 0.981932411; 48 | SLE GWAS | 3 | Coronary
artery disease || id: ebi-a-GCST005194 | Penalised weighted
median | 3 | 0.380340445 | 74.03252452 | 0.995900904; 48 | SLE GWAS | 3 | Coronary
artery disease || id: ebi-a-GCST005194 | Inverse variance
weighted | 3 | 0.567463513 | 0.245872514 | 0.021001455; 48 | SLE GWAS | 3 | Coronary
artery disease || id: ebi-a-GCST005194 | Inverse variance weighted (multiplicative random
effects) | 3 | 0.567463513 | 0.245872514 | 0.021001455; 48 | SLE GWAS | 3 | Coronary
artery disease || id: ebi-a-GCST005194 | Inverse variance weighted (fixed
effects) | 3 | 0.567463513 | 0.087998352 | 1.12911E−10; 48 | SLE GWAS | 3 | Coronary
artery disease || id: ebi-a-GCST005194 | Simple
mode | 3 | 0.803248111 | 0.545393441 | 0.278695506; 48 | SLE GWAS | 3 | Coronary
artery disease || id: ebi-a-GCST005194 | Weighted
mode | 3 | 0.671205089 | 0.418146515 | 0.249668038; 48 | SLE GWAS | 3 | Coronary
artery disease || id: ebi-a-GCST005194 | Weighted mode
(NOME) | 3 | 0.671205089 | 0.098164079 | 0.020726543; 48 | SLE GWAS | 3 | Coronary
artery disease || id: ebi-a-GCST005194 | Simple mode
(NOME) | 3 | 0.803248111 | 0.133059789 | 0.026360449; 48 | SLE GWAS | 3 | Coronary
artery disease || id: ebi-a-GCST005194 | Robust adjusted profile score
(RAPS) | 3 | 0.875019225 | 0.63323209 | 0.167023844; 49 | SLE GWAS | 32 | Coronary
artery disease || id: ebi-a-GCST005194 | Maximum likelihood | 27 | 1.0404E−05
| 0.012578096 | 0.999340025; 49 | SLE GWAS | 32 | Coronary artery disease || id: ebi-a-
GCST005194 | MR Egger | 27 | 0.012293741 | 0.029702151 | 0.682476293; 49 | SLE
GWAS | 32 | Coronary artery disease || id: ebi-a-GCST005194 | MR Egger
(bootstrap) | 27 | 0.014268183 | 0.026214371 | 0.296; 49 | SLE GWAS | 32 | Coronary
artery disease || id: ebi-a-GCST005194 | Simple median | 27
| −0.040597044 | 0.0294635 | 0.168241629; 49 | SLE GWAS | 32 | Coronary artery disease ||
id: ebi-a-GCST005194 | Weighted median | 27 | 0.008515617 | 0.015104883 | 0.572913531;
49 | SLE GWAS | 32 | Coronary artery disease || id: ebi-a-GCST005194 | Penalised
weighted median | 27 | 0.008486451 | 0.014503695 | 0.558464784; 49 | SLE
GWAS | 32 | Coronary artery disease || id: ebi-a-GCST005194 | Inverse variance
weighted | 27 | 1.0404E−05 | 0.019724438 | 0.99957914; 49 | SLE GWAS | 32 | Coronary
artery disease || id: ebi-a-GCST005194 | Inverse variance weighted (multiplicative random
effects) | 27 | 1.0404E−05 | 0.019724438 | 0.99957914; 49 | SLE GWAS | 32 | Coronary
artery disease || id: ebi-a-GCST005194 | Inverse variance weighted (fixed
effects) | 27 | 1.0404E−05 | 0.010931707 | 0.999240628; 49 | SLE GWAS | 32 | Coronary
artery disease || id: ebi-a-GCST005194 | Simple
mode | 27 | 0.016021277 | 0.034127567 | 0.642659346; 49 | SLE GWAS | 32 | Coronary
artery disease || id: ebi-a-GCST005194 | Weighted
mode | 27 | 0.00919211 | 0.013916885 | 0.514742551; 49 | SLE GWAS | 32 | Coronary
artery disease || id: ebi-a-GCST005194 | Weighted mode
(NOME) | 27 | 0.016021277 | 0.037576366 | 0.673350741; 49 | SLE GWAS | 32 | Coronary
artery disease || id: ebi-a-GCST005194 | Simple mode
(NOME) | 27 | 0.00919211 | 0.014793097 | 0.53976106; 49 | SLE GWAS | 32 | Coronary
artery disease || id: ebi-a-GCST005194 | Robust adjusted profile score (RAPS) | 27
| −0.006362768 | 0.01991155 | 0.749308245; 49 | SLE GWAS | 32 | Coronary artery disease ||
id: ebi-a-GCST005194 | Raw | 27 | 1.0404E−05 | 0.019724438 | 0.999583166; 49 | SLE
GWAS | 32 | Coronary artery disease || id: ebi-a-GCST005194 | Outlier-corrected | 25
| −0.015115672 | 0.01589095 | 0.350978339; 50 | SLE GWAS | 39 | Coronary artery disease ||
id: ebi-a-GCST005194 | Maximum likelihood | 35
| −0.063920739 | 0.006220487 | 9.05501E−25; 50 | SLE GWAS | 39 | Coronary artery disease
|| id: ebi-a-GCST005194 | MR Egger | 35 | −0.076950434 | 0.02131413 | 0.001001687;
50 | SLE GWAS | 39 | Coronary artery disease || id: ebi-a-GCST005194 | MR Egger
(bootstrap) | 35 | −0.051910753 | 0.01900237 | 0.001; 50 | SLE GWAS | 39 | Coronary
artery disease || id: ebi-a-GCST005194 | Simple median | 35
| −0.06453245 | 0.008330183 | 9.42205E−15; 50 | SLE GWAS | 39 | Coronary artery disease ||
id: ebi-a-GCST005194 | Weighted median | 35 | −0.064259217 | 0.00853977 | 5.28411E−14;
50 | SLE GWAS | 39 | Coronary artery disease || id: ebi-a-GCST005194 | Penalised
weighted median | 35 | −0.064335111 | 0.007963117 | 6.52267E−16; 50 | SLE
GWAS | 39 | Coronary artery disease || id: ebi-a-GCST005194 | Inverse variance
weighted | 35 | −0.062716572 | 0.005836664 | 6.23744E−27; 50 | SLE
GWAS | 39 | Coronary artery disease || id: ebi-a-GCST005194 | Inverse variance weighted
(multiplicative random effects) | 35 | −0.062716572 | 0.004320432 | 9.55668E−48; 50 | SLE
GWAS | 39 | Coronary artery disease || id: ebi-a-GCST005194 | Inverse variance weighted
(fixed effects) | 35 | −0.062716572 | 0.005836664 | 6.23744E−27; 50 | SLE
GWAS | 39 | Coronary artery disease || id: ebi-a-GCST005194 | Simple mode | 35
| −0.071239725 | 0.014048051 | 1.38907E−05; 50 | SLE GWAS | 39 | Coronary artery disease
|| id: ebi-a-GCST005194 | Weighted mode | 35 | −0.070525373 | 0.014443618 | 2.43589E−05;
50 | SLE GWAS | 39 | Coronary artery disease || id: ebi-a-GCST005194 | Weighted
mode (NOME) | 35 | −0.070525373 | 0.01254822 | 2.6827E−06; 50 | SLE
GWAS | 39 | Coronary artery disease || id: ebi-a-GCST005194 | Simple mode
(NOME) | 35 | −0.071239725 | 0.013277049 | 5.75511E−06; 50 | SLE
GWAS | 39 | Coronary artery disease || id: ebi-a-GCST005194 | Robust adjusted profile
score (RAPS) | 35 | −0.064698498 | 0.006408648 | 5.78359E−24; 50 | SLE
GWAS | 39 | Coronary artery disease || id: ebi-a-GCST005194 | Raw | 35
| −0.062716572 | 0.004320432 | 3.90173E−16; 50 | SLE GWAS | 39 | Coronary artery disease
|| id: ebi-a-GCST005194 | Outlier-corrected | NA | NA | NA | NA; 51 | SLE
GWAS | 5 | Coronary artery disease || id: ebi-a-GCST005194 | Maximum
likelihood | 4 | 0.144184674 | 0.059604007 | 0.015561387; 51 | SLE GWAS | 5 | Coronary
artery disease || id: ebi-a-GCST005194 | MR
Egger | 4 | 0.153374352 | 0.235183979 | 0.58124236; 51 | SLE GWAS | 5 | Coronary artery
disease || id: ebi-a-GCST005194 | MR Egger | (bootstrap) | 4
| −0.006221992 | 0.181779888 | 0.494; 51 | SLE GWAS | 5 | Coronary artery disease || id: ebi-
a-GCST005194 | Simple median | 4 | 0.138325048 | 0.076700529 | 0.071318637; 51 | SLE
GWAS | 5 | Coronary artery disease || id: ebi-a-GCST005194 | Weighted
median | 4 | 0.059851214 | 1.295078152 | 0.963139393; 51 | SLE GWAS | 5 | Coronary
artery disease || id: ebi-a-GCST005194 | Penalised weighted
median | 4 | 0.059851214 | 0.102656608 | 0.559877488; 51 | SLE GWAS | 5 | Coronary
artery disease || id: ebi-a-GCST005194 | Inverse variance
weighted | 4 | 0.12276036 | 0.046998289 | 0.00900096; 51 | SLE GWAS | 5 | Coronary
artery disease || id: ebi-a-GCST005194 | Inverse variance weighted (multiplicative random
effects) | 4 | 0.12276036 | 0.046998289 | 0.00900096; 51 | SLE GWAS | 5 | Coronary artery
disease || id: ebi-a-GCST005194 | Inverse variance weighted (fixed
effects) | 4 | 0.12276036 | 0.04308682 | 0.004383766; 51 | SLE GWAS | 5 | Coronary artery
disease || id: ebi-a-GCST005194 | Simple
mode | 4 | 0.206595221 | 0.090989228 | 0.107870758; 51 | SLE GWAS | 5 | Coronary
artery disease || id: ebi-a-GCST005194 | Weighted
mode | 4 | 0.055343727 | 0.065994818 | 0.46324792; 51 | SLE GWAS | 5 | Coronary artery
disease || id: ebi-a-GCST005194 | Weighted mode
(NOME) | 4 | 0.058655804 | 0.073558602 | 0.48349751; 51 | SLE GWAS | 5 | Coronary
artery disease || id: ebi-a-GCST005194 | Simple mode
(NOME) | 4 | 0.206595221 | 0.089591557 | 0.104415776; 51 | SLE GWAS | 5 | Coronary
artery disease || id: ebi-a-GCST005194 | Robust adjusted profile score
(RAPS) | 4 | 0.144206127 | 0.061332715 | 0.018712442; 51 | SLE GWAS | 5 | Coronary
artery disease || id: ebi-a-
GCST005194 | Raw | 4 | 0.12276036 | 0.046998289 | 0.079546476; 51 | SLE
GWAS | 5 | Coronary artery disease || id: ebi-a-GCST005194 | Outlier-
corrected | NA | NA | NA | NA; 52 | SLE GWAS | 8 | Coronary artery disease || id: ebi-a-
GCST005194 | Maximum likelihood | 8 | 0.004332126 | 0.021142442 | 0.837648787;
52 | SLE GWAS | 8 | Coronary artery disease || id: ebi-a-GCST005194 | MR Egger | 8
| −0.025945212 | 0.057127427 | 0.665677134; 52 | SLE GWAS | 8 | Coronary artery disease ||
id: ebi-a-GCST005194 | MR Egger | (bootstrap) | 8 | −0.021375486 | 0.08959224 | 0.4;
52 | SLE GWAS | 8 | Coronary artery disease || id: ebi-a-GCST005194 | Simple
median | 8 | 0.015208247 | 0.027069848 | 0.574242065; 52 | SLE GWAS | 8 | Coronary
artery disease || id: ebi-a-GCST005194 | Weighted
median | 8 | 0.009960033 | 0.026559621 | 0.707655552; 52 | SLE GWAS | 8 | Coronary
artery disease || id: ebi-a-GCST005194 | Penalised weighted
median | 8 | 0.010144929 | 0.025519889 | 0.690976464; 52 | SLE GWAS | 8 | Coronary
artery disease || id: ebi-a-GCST005194 | Inverse variance
weighted | 8 | 0.003772197 | 0.025975282 | 0.884535166; 52 | SLE GWAS | 8 | Coronary
artery disease || id: ebi-a-GCST005194 | Inverse variance weighted (multiplicative random
effects) | 8 | 0.003772197 | 0.025975282 | 0.884535166; 52 | SLE GWAS | 8 | Coronary
artery disease || id: ebi-a-GCST005194 | Inverse variance weighted (fixed
effects) | 8 | 0.003772197 | 0.019788589 | 0.848819532; 52 | SLE GWAS | 8 | Coronary
artery disease || id: ebi-a-GCST005194 | Simple
mode | 8 | 0.013173625 | 0.032191095 | 0.694601089; 52 | SLE GWAS | 8 | Coronary
artery disease || id: ebi-a-GCST005194 | Weighted
mode | 8 | 0.008235886 | 0.02625827 | 0.76292731; 52 | SLE GWAS | 8 | Coronary artery
disease || id: ebi-a-GCST005194 | Weighted mode
(NOME) | 8 | 0.008235886 | 0.026963053 | 0.768905812; 52 | SLE GWAS | 8 | Coronary
artery disease || id: ebi-a-GCST005194 | Simple mode
(NOME) | 8 | 0.013173625 | 0.031616927 | 0.689404034; 52 | SLE GWAS | 8 | Coronary
artery disease || id: ebi-a-GCST005194 | Robust adjusted profile score
(RAPS) | 8 | 0.011253156 | 0.024044992 | 0.639781638; 52 | SLE GWAS | 8 | Coronary
artery disease || id: ebi-a-
GCST005194 | Raw | 8 | 0.003772197 | 0.025975282 | 0.888628285; 52 | SLE
GWAS | 8 | Coronary artery disease || id: ebi-a-GCST005194 | Outlier-
corrected | NA NA NA NA; 53 | SLE GWAS | 9 | Coronary artery disease || id: ebi-a-
GCST005194 | Maximum likelihood | 7 | 0.010476033 | 0.030912329 | 0.734688858;
53 | SLE GWAS | 9 | Coronary artery disease || id: ebi-a-GCST005194 | MR Egger | 7
| −0.015744234 | 0.070891177 | 0.833030048; 53 | SLE GWAS | 9 | Coronary artery disease ||
id: ebi-a-GCST005194 | MR Egger | (bootstrap) | 7 | 0.046894013 | 0.102980114 | 0.322;
53 | SLE GWAS | 9 | Coronary artery disease || id: ebi-a-GCST005194 | Simple
median | 7 | 0.062008503 | 0.05169046 | 0.230290017; 53 | SLE GWAS | 9 | Coronary
artery disease || id: ebi-a-GCST005194 | Weighted median | 7
| −0.01772272 | 0.040021336 | 0.657887404; 53 | SLE GWAS | 9 | Coronary artery disease ||
id: ebi-a-GCST005194 | Penalised weighted median | 7
| −0.01772272 | 0.040133799 | 0.658785279; 53 | SLE GWAS | 9 | Coronary artery disease ||
id: ebi-a-GCST005194 | Inverse variance
weighted | 7 | 0.008760306 | 0.033890588 | 0.796030399; 53 | SLE GWAS | 9 | Coronary
artery disease || id: ebi-a-GCST005194 | Inverse variance weighted (multiplicative random
effects) | 7 | 0.008760306 | 0.033890588 | 0.796030399; 53 | SLE GWAS | 9 | Coronary
artery disease || id: ebi-a-GCST005194 | Inverse variance weighted (fixed
effects) | 7 | 0.008760306 | 0.028295913 | 0.756868069; 53 | SLE GWAS | 9 | Coronary
artery disease || id: ebi-a-GCST005194 | Simple
mode | 7 | 0.053116966 | 0.049378597 | 0.323398932; 53 | SLE GWAS | 9 | Coronary
artery disease || id: ebi-a-GCST005194 | Weighted mode | 7
| −0.001078478 | 0.036895057 | 0.977628432; 53 | SLE GWAS | 9 | Coronary artery disease ||
id: ebi-a-GCST005194 | Weighted mode
(NOME) | 7 | 0.004943238 | 0.038583616 | 0.90224228; 53 | SLE GWAS | 9 | Coronary
artery disease || id: ebi-a-GCST005194 | Simple mode
(NOME) | 7 | 0.053116966 | 0.048100813 | 0.311776046; 53 | SLE GWAS | 9 | Coronary
artery disease || id: ebi-a-GCST005194 | Robust adjusted profile score
(RAPS) | 7 | 0.008904427 | 0.037479131 | 0.812204077; 53 | SLE GWAS | 9 | Coronary
artery disease || id: ebi-a-
GCST005194 | Raw | 7 | 0.008760306 | 0.033890588 | 0.804668984; 53 | SLE
GWAS | 9 | Coronary artery disease || id: ebi-a-GCST005194 | Outlier-
corrected | NA | NA | NA | NA; 54 | SLE GWAS | 4 | Coronary artery disease || id: ebi-a-
GCST005194 | Maximum likelihood | 4 | −0.004413443 | 0.039121148 | 0.91017741;
54 | SLE GWAS | 4 | Coronary artery disease || id: ebi-a-GCST005194 | MR Egger | 4
| −0.031019418 | 0.109836283 | 0.804169024; 54 | SLE GWAS | 4 | Coronary artery disease
id: ebi-a-GCST005194 | MR Egger | (bootstrap) | 4 | −0.006995239 | 0.075167927 | 0.457;
54 | SLE GWAS | 4 | Coronary artery disease || id: ebi-a-GCST005194 | Simple
median | 4 | −0.012280399 | 0.074419371 | 0.868931266; 54 | SLE GWAS | 4 | Coronary
artery disease || id: ebi-a-GCST005194 | Weighted median | 4
| −0.012413382 | 0.050809363 | 0.806988511; 54 | SLE GWAS | 4 | Coronary artery disease ||
id: ebi-a-GCST005194 | Penalised weighted median | 4
| −0.012413382 | 0.050791161 | 0.806920709; 54 | SLE GWAS | 4 | Coronary artery disease ||
id: ebi-a-GCST005194 | Inverse variance weighted | 4
| −0.004204122 | 0.038213587 | 0.912396355; 54 | SLE GWAS | 4 | Coronary artery disease ||
id: ebi-a-GCST005194 | Inverse variance weighted (multiplicative random effects) | 4
| −0.004204122 | 0.025514887 | 0.869123969; 54 | SLE GWAS | 4 | Coronary artery disease ||
id: ebi-a-GCST005194 | Inverse variance weighted (fixed effects) | 4
| −0.004204122 | 0.038213587 | 0.912396355; 54 | SLE GWAS | 4 | Coronary artery disease ||
id: ebi-a-GCST005194 | Simple mode | 4 | −0.01833099 | 0.063354626 | 0.791165231;
54 | SLE GWAS | 4 | Coronary artery disease || id: ebi-a-GCST005194 | Weighted
mode | 4 | −0.013200403 | 0.049759175 | 0.807973897; 54 | SLE GWAS | 4 | Coronary
artery disease || id: ebi-a-GCST005194 | Weighted mode (NOME) | 4
| −0.013200403 | 0.047897298 | 0.800749868; 54 | SLE GWAS | 4 | Coronary artery disease ||
id: ebi-a-GCST005194 | Simple mode (NOME) | 4
| −0.01833099 | 0.060878256 | 0.78299503; 54 | SLE GWAS | 4 | Coronary artery disease ||
id: ebi-a-GCST005194 | Robust adjusted profile score (RAPS) | 4
| −0.004405884 | 0.041556193 | 0.915564638; 54 | SLE GWAS | 4 | Coronary artery disease ||
id: ebi-a-GCST005194 | Raw | 4 | −0.004204122 | 0.025514887 | 0.879600609; 54 | SLE
GWAS | 4 | Coronary artery disease || id: ebi-a-GCST005194 | Outlier-
corrected | NA | NA | NA | NA; 55 | SLE GWAS | 27 | Coronary artery disease || id: ebi-a-
GCST005194 | Maximum likelihood | 25 | 0.058443345 | 0.014869103 | 8.47614E−05;
55 | SLE GWAS | 27 | Coronary artery disease || id: ebi-a-GCST005194 | MR
Egger | 25 | 0.006482464 | 0.036672654 | 0.861239837; 55 | SLE GWAS | 27 | Coronary
artery disease || id: ebi-a-GCST005194 | MR Egger
(bootstrap) | 25 | 0.021221856 | 0.033474328 | 0.275; 55 | SLE GWAS | 27 | Coronary
artery disease || id: ebi-a-GCST005194 | Simple
median | 25 | 0.058346581 | 0.028938139 | 0.04377363; 55 | SLE GWAS | 27 | Coronary
artery disease || id: ebi-a-GCST005194 | Weighted
median | 25 | 0.006547076 | 0.017494546 | 0.708229307; 55 | SLE GWAS | 27 | Coronary
artery disease || id: ebi-a-GCST005194 | Penalised weighted
median | 25 | 0.005493102 | 0.017580288 | 0.75469246; 55 | SLE GWAS | 27 | Coronary
artery disease || id: ebi-a-GCST005194 | Inverse variance
weighted | 25 | 0.041391273 | 0.020953062 | 0.048219552; 55 | SLE GWAS | 27 | Coronary
artery disease || id: ebi-a-GCST005194 | Inverse variance weighted (multiplicative random
effects) | 25 | 0.041391273 | 0.020953062 | 0.048219552; 55 | SLE GWAS | 27 | Coronary
artery disease || id: ebi-a-GCST005194 | Inverse variance weighted (fixed
effects) | 25 | 0.041391273 | 0.011801502 | 0.000452698; 55 | SLE GWAS | 27 | Coronary
artery disease || id: ebi-a-GCST005194 | Simple
mode | 25 | 0.043686513 | 0.027613291 | 0.126720976; 55 | SLE GWAS | 27 | Coronary
artery disease || id: ebi-a-GCST005194 | Weighted
mode | 25 | 0.008163952 | 0.01586914 | 0.611639424; 55 | SLE GWAS | 27 | Coronary
artery disease || id: ebi-a-GCST005194 | Weighted mode
(NOME) | 25 | 0.008163952 | 0.015932351 | 0.613045384; 55 | SLE GWAS | 27 | Coronary
artery disease || id: ebi-a-GCST005194 | Simple mode
(NOME) | 25 | 0.043686513 | 0.03037487 | 0.163277746; 55 | SLE GWAS | 27 | Coronary
artery disease || id: ebi-a-GCST005194 | Robust adjusted profile score
(RAPS) | 25 | 0.031937499 | 0.023277008 | 0.170044115; 55 | SLE GWAS | 27 | Coronary
artery disease || id: ebi-a-
GCST005194 | Raw | 25 | 0.041391273 | 0.020953062 | 0.05982754; 55 | SLE
GWAS | 27 | Coronary artery disease || id: ebi-a-GCST005194 | Outlier-
corrected | 23 | 0.016158975 | 0.018995795 | 0.404121664; 56 | SLE
GWAS | 19 | Coronary artery disease || id: ebi-a-GCST005194 | Maximum likelihood | 16
| −0.0439015 | 0.018842021 | 0.019807299; 56 | SLE GWAS | 19 | Coronary artery disease ||
id: ebi-a-GCST005194 | MR Egger | 16 | −0.035949495 | 0.037588971 | 0.355102681;
56 | SLE GWAS | 19 | Coronary artery disease || id: ebi-a-GCST005194 | MR Egger
(bootstrap) | 16 | −0.055599106 | 0.030467613 | 0.029; 56 | SLE GWAS | 19 | Coronary
artery disease || id: ebi-a-GCST005194 | Simple median | 16
| −0.036034263 | 0.036499828 | 0.323522672; 56 | SLE GWAS | 19 | Coronary artery disease
|| id: ebi-a-GCST005194 | Weighted median | 16
| −0.041324625 | 0.023687232 | 0.081055407; 56 | SLE GWAS | 19 | Coronary artery disease
|| id: ebi-a-GCST005194 | Penalised weighted median | 16
| −0.041324625 | 0.023333964 | 0.076559449; 56 | SLE GWAS | 19 | Coronary artery disease
|| id: ebi-a-GCST005194 | Inverse variance weighted | 16
| −0.036688513 | 0.022159142 | 0.097786051; 56 | SLE GWAS | 19 | Coronary artery disease
|| id: ebi-a-GCST005194 | Inverse variance weighted (multiplicative random effects) | 16
| −0.036688513 | 0.022159142 | 0.097786051; 56 | SLE GWAS | 19 | Coronary artery disease
|| id: ebi-a-GCST005194 | Inverse variance weighted (fixed effects) | 16
| −0.036688513 | 0.0167329 | 0.028336395; 56 | SLE GWAS | 19 | Coronary artery disease ||
id: ebi-a-GCST005194 | Simple mode | 16 | −0.026117491 | 0.041386089 | 0.537493248;
56 | SLE GWAS | 19 | Coronary artery disease || id: ebi-a-GCST005194 | Weighted
mode | 16 | −0.036311532 | 0.020689276 | 0.099640318; 56 | SLE GWAS | 19 | Coronary
artery disease || id: ebi-a-GCST005194 | Weighted mode (NOME) | 16
0| −.041408552 | 0.020561452 | 0.062319056; 56 | SLE GWAS | 19 | Coronary artery disease
|| id: ebi-a-GCST005194 | Simple mode (NOME) | 16
| −0.026117491 | 0.040533386 | 0.52908264; 56 | SLE GWAS | 19 | Coronary artery disease ||
id: ebi-a-GCST005194 | Robust adjusted profile score (RAPS) | 16
| −0.039341396 | 0.025403904 | 0.121469289; 56 | SLE GWAS | 19 | Coronary artery disease
|| id: ebi-a-GCST005194 | Raw | 16 | −0.036688513 | 0.022159142 | 0.1185514; 56 | SLE
GWAS | 19 | Coronary artery disease || id: ebi-a-GCST005194 | Outlier-
corrected | NA | NA | NA | NA; 57 | SLE GWAS | 27 | Coronary artery disease || id: ebi-a-
GCST005194 | Maximum likelihood | 25 | 0.049924111 | 0.011627944 | 1.7591E−05;
57 | SLE GWAS | 27 | Coronary artery disease || id: ebi-a-GCST005194 | MR
Egger | 25 | 0.032288249 | 0.034885682 | 0.36429289; 57 | SLE GWAS | 27 | Coronary
artery disease || id: ebi-a-GCST005194 | MR Egger
(bootstrap) | 25 | 0.045953617 | 0.020549359 | 0.014; 57 | SLE GWAS | 27 | Coronary
artery disease || id: ebi-a-GCST005194 | Simple
median | 25 | 0.016833366 | 0.023675126 | 0.477075025; 57 | SLE GWAS | 27 | Coronary
artery disease || id: ebi-a-GCST005194 | Weighted
median | 25 | 0.017009252 | 0.012661741 | 0.179156051; 57 | SLE GWAS | 27 | Coronary
artery disease || id: ebi-a-GCST005194 | Penalised weighted
median | 25 | 0.017005856 | 0.013159357 | 0.196252767; 57 | SLE GWAS | 27 | Coronary
artery disease || id: ebi-a-GCST005194 | Inverse variance
weighted | 25 | 0.032334522 | 0.022722192 | 0.154725399; 57 | SLE GWAS | 27 | Coronary
artery disease || id: ebi-a-GCST005194 | Inverse variance weighted (multiplicative random
effects) | 25 | 0.032334522 | 0.022722192 | 0.154725399; 57 | SLE GWAS | 27 | Coronary
artery disease || id: ebi-a-GCST005194 | Inverse variance weighted (fixed
effects) | 25 | 0.032334522 | 0.009321488 | 0.000522759; 57 | SLE GWAS | 27 | Coronary
artery disease || id: ebi-a-GCST005194 | Simple mode | 25
| −0.017555901 | 0.028292722 | 0.540770628; 57 | SLE GWAS | 27 | Coronary artery disease
|| id: ebi-a-GCST005194 | Weighted mode | 25 | 0.01604482 | 0.013023286 | 0.229879902;
57 | SLE GWAS | 27 | Coronary artery disease || id: ebi-a-GCST005194 | Weighted mode
(NOME) | 25 | 0.01604482 | 0.011261509 | 0.167104729; 57 | SLE GWAS | 27 | Coronary
artery disease || id: ebi-a-GCST005194 | Simple mode (NOME) | 25
| −0.017555901 | 0.025994997 | 0.505904427; 57 | SLE GWAS | 27 | Coronary artery disease
|| id: ebi-a-GCST005194 | Robust adjusted profile score
(RAPS) | 25 | 0.019707352 | 0.021670208 | 0.363127702; 57 | SLE GWAS | 27 | Coronary
artery disease || id: ebi-a-
GCST005194 | Raw | 25 | 0.032334522 | 0.022722192 | 0.167595152; 57 | SLE
GWAS | 27 | Coronary artery disease || id: ebi-a-GCST005194 | Outlier-
corrected | 23 | 0.007081578 | 0.014676658 | 0.634212602; 58 | SLE
GWAS | 10 | Coronary artery disease || id: ebi-a-GCST005194 | Maximum
likelihood | 8 | 0.078110366 | 0.065190308 | 0.230843106; 58 | SLE GWAS | 10 | Coronary
artery disease || id: ebi-a-GCST005194 | MR Egger | 8
| −0.09678514 | 0.165028521 | 0.578936603; 58 | SLE GWAS | 10 | Coronary artery disease ||
id: ebi-a-GCST005194 | MR Egger | (bootstrap) | 8 | −0.102518491 | 0.145770797 | 0.21;
58 | SLE GWAS | 10 | Coronary artery disease || id: ebi-a-GCST005194 | Simple
median | 8 | 0.156013204 | 0.083103594 | 0.06047233; 58 | SLE GWAS | 10 | Coronary
artery disease || id: ebi-a-GCST005194 | Weighted
median | 8 | 0.024656239 | 0.07894727 | 0.754802856; 58 | SLE GWAS | 10 | Coronary
artery disease || id: ebi-a-GCST005194 | Penalised weighted
median | 8 | 0.025273143 | 0.080167913 | 0.752569874; 58 | SLE GWAS | 10 | Coronary
artery disease || id: ebi-a-GCST005194 | Inverse variance
weighted | 8 | 0.019084815 | 0.070203235 | 0.78573664; 58 | SLE GWAS | 10 | Coronary
artery disease || id: ebi-a-GCST005194 | Inverse variance weighted | (multiplicative random
effects) | 8 | 0.019084815 | 0.070203235 | 0.78573664; 58 | SLE GWAS | 10 | Coronary
artery disease || id: ebi-a-GCST005194 | Inverse variance weighted (fixed
effects) | 8 || 0.019084815 | 0.035002543 | 0.585587901; 58 | SLE GWAS | 10 | Coronary
artery disease || id: ebi-a-GCST005194 | Simple
mode | 8 | 0.184471332 | 0.127553407 | 0.191358983; 58 | SLE GWAS | 10 | Coronary
artery disease || id: ebi-a-GCST005194 | Weighted
mode | 8 | 0.115674898 | 0.090449403 | 0.241698063; 58 | SLE GWAS | 10 | Coronary
artery disease || id: ebi-a-GCST005194 | Weighted mode
(NOME) | 8 | 0.161539187 | 0.121535994 | 0.225482457; 58 | SLE GWAS | 10 | Coronary
artery disease || id: ebi-a-GCST005194 | Simple mode
(NOME) | 8 | 0.184471332 | 0.107367835 | 0.129469894; 58 | SLE GWAS | 10 | Coronary
artery disease || id: ebi-a-GCST005194 | Robust adjusted profile score
(RAPS) | 8 | 0.062232613 | 0.096369186 | 0.518426414; 58 | SLE GWAS | 10 | Coronary
artery disease || id: ebi-a-
GCST005194 | Raw | 8 | 0.019084815 | 0.070203235 | 0.793580013; 58 | SLE
GWAS | 10 | Coronary artery disease || id: ebi-a-GCST005194 | Outlier-
corrected | 5 | 0.150194277 | 0.054967194 | 0.052311519; 59 | SLE GWAS | 8 | Coronary
artery disease || id: ebi-a-GCST005194 | Maximum
likelihood | 8 | 0.087228984 | 0.055424295 | 0.115524398; 59 | SLE GWAS | 8 | Coronary
artery disease || id: ebi-a-GCST005194 | MR
Egger | 8 || 0.085857146 | 0.109260636 | 0.461878799; 59 | SLE GWAS | 8 | Coronary
artery disease || id: ebi-a-GCST005194 | MR Egger
(bootstrap) | 8 | 0.052734293 | 0.109874792 | 0.303; 59 | SLE GWAS | 8 | Coronary artery
disease || id: ebi-a-GCST005194 | Simple
median | 8 | 0.016803152 | 0.094240565 || 0.858486887; 59 | SLE GWAS | 8 | Coronary
artery disease || id: ebi-a-GCST005194 | Weighted
median | 8 | 0.012837184 | 0.076943506 | 0.86749666; 59 | SLE GWAS | 8 | Coronary
artery disease || id: ebi-a-GCST005194 | Penalised weighted
median | 8 | 0.012837184 | 0.073987725 | 0.862255091; 59 | SLE GWAS | 8 | Coronary
artery disease || id: ebi-a-GCST005194 | Inverse variance
weighted | 8 | 0.05249497 | 0.055716111 | 0.346097141; 59 | SLE GWAS | 8 | Coronary
artery disease || id: ebi-a-GCST005194 | Inverse variance weighted (multiplicative random
effects) | 8 | 0.05249497 | 0.055716111 | 0.346097141; 59 | SLE GWAS | 8 | Coronary
artery disease || id: ebi-a-GCST005194 | Inverse variance weighted (fixed
effects) | 8 | 0.05249497 | 0.041263856 | 0.203309882; 59 | SLE GWAS | 8 | Coronary
artery disease || id: ebi-a-GCST005194 | Simple
mode | 8 | 0.085654662 | 0.099181095 | 0.416404563; 59 | SLE GWAS | 8 | Coronary
artery disease || id: ebi-a-GCST005194 | Weighted
mode | 8 | 0.01171503 | 0.060636102 | 0.852286879; 59 | SLE GWAS | 8 | Coronary artery
disease || id: ebi-a-GCST005194 | Weighted mode
(NOME) | 8 | 0.042847507 | 0.066880831 | 0.542143206; 59 | SLE GWAS | 8 | Coronary
artery disease || id: ebi-a-GCST005194 | Simple mode
(NOME) | 8 | 0.085654662 | 0.098404464 | 0.412914771; 59 | SLE GWAS | 8 | Coronary
artery disease || id: ebi-a-GCST005194 | Robust adjusted profile score
(RAPS) | 8 | 0.075722514 | 0.08608801 | 0.37907918; 59 | SLE GWAS | 8 | Coronary
artery disease || id: ebi-a-
GCST005194 | Raw | 8 | 0.05249497 | 0.055716111 | 0.377468281; 59 | SLE
GWAS | 8 | Coronary artery disease || id: ebi-a-GCST005194 | Outlier-
corrected | NA | NA | NA | NA; 60 | SLE GWAS | 4 | Coronary artery disease || id: ebi-a-
GCST005194 | Maximum likelihood | 4 | 0.262408698 | 0.11145097 | 0.018548813;
60 | SLE GWAS | 4 | Coronary artery disease || id: ebi-a-GCST005194 | MR
Egger | 4 | 0.226084435 | 0.189201187 | 0.354593098; 60 | SLE GWAS | 4 | Coronary
artery disease || id: ebi-a-GCST005194 | MR Egger
(bootstrap) | 4 | 0.166378192 | 0.271746588 | 0.119; 60 | SLE GWAS | 4 | Coronary artery
disease || id: ebi-a-GCST005194 | Simple
median | 4 | 0.275087253 | 0.249558494 | 0.27033314; 60 | SLE GWAS | 4 | Coronary
artery disease || id: ebi-a-GCST005194 | Weighted
median | 4 | 0.163989456 | 1.084258114 | 0.879781844; 60 | SLE GWAS | 4 | Coronary
artery disease || id: ebi-a-GCST005194 | Penalised weighted
median | 4 | 0.163989456 | 0.280199195 | 0.558372725; 60 | SLE GWAS | 4 | Coronary
artery disease || id: ebi-a-GCST005194 | Inverse variance
weighted | 4 | 0.212939365 | 0.077515063 | 0.006013016; 60 | SLE GWAS | 4 | Coronary
artery disease || id: ebi-a-GCST005194 | Inverse variance weighted (multiplicative random
effects) | 4 | 0.212939365 | 0.077515063 | 0.006013016; 60 | SLE GWAS | 4 | Coronary
artery disease || id: ebi-a-GCST005194 | Inverse variance weighted (fixed
effects) | 4 | 0.212939365 | 0.059380797 | 0.000335793; 60 | SLE GWAS | 4 | Coronary
artery disease || id: ebi-a-GCST005194 | Simple
mode | 4 | 0.342089193 | 0.171183922 | 0.139545957; 60 | SLE GWAS | 4 | Coronary
artery disease || id: ebi-a-GCST005194 | Weighted
mode | 4 | 0.188659613 | 0.095418697 | 0.142448136; 60 | SLE GWAS | 4 | Coronary
artery disease || id: ebi-a-GCST005194 | Weighted mode
(NOME) | 4 | 0.200461888 | 0.067998934 | 0.060120637; 60 | SLE GWAS | 4 | Coronary
artery disease || id: ebi-a-GCST005194 | Simple mode
(NOME) | 4 | 0.342089193 | 0.154361274 | 0.113448277; 60 | SLE GWAS | 4 | Coronary
artery disease || id: ebi-a-GCST005194 | Robust adjusted profile score
(RAPS) | 4 | 0.262448283 | 0.121670339 | 0.03100223; 60 | SLE GWAS | 4 | Coronary
artery disease || id: ebi-a-
GCST005194 | Raw | 4 | 0.212939365 | 0.077515063 | 0.070916218; 60 | SLE
GWAS | 4 | Coronary artery disease || id: ebi-a-GCST005194 | Outlier-
corrected | NA NA NA NA; 61 | SLE GWAS | 79 | Coronary artery disease || id: ebi-a-
GCST005194 | Maximum likelihood | 71 | −0.017829126 | 0.004469997 | 6.64584E−05;
61 | SLE GWAS | 79 | Coronary artery disease || id: ebi-a-GCST005194 | MR Egger | 71
| −0.025769648 | 0.011425378 | 0.027277774; 61 | SLE GWAS | 79 | Coronary artery disease
|| id: ebi-a-GCST005194 | MR Egger | (bootstrap) | 71 −0.00929326 | 0.013076168 | 0.242;
61 | SLE GWAS | 79 | Coronary artery disease || id: ebi-a-GCST005194 | Simple
median | 71 | −0.020254389 | 0.006566096 | 0.002037624; 61 | SLE GWAS | 79 | Coronary
artery disease || id: ebi-a-GCST005194 | Weighted median | 71
| −0.028053045 | 0.00615565 | 5.18193E−06; 61 | SLE GWAS | 79 | Coronary artery disease ||
id: ebi-a-GCST005194 | Penalised weighted median | 71
| −0.028053045 | 0.005736762 | 1.00811E−06; 61 | SLE GWAS | 79 | Coronary artery disease
|| id: ebi-a-GCST005194 | Inverse variance weighted | 71
| −0.0174862 | 0.004621084 | 0.000154325; 61 | SLE GWAS | 79 | Coronary artery disease ||
id: ebi-a-GCST005194 | Inverse variance weighted (multiplicative random effects) | 71
| −0.0174862 | 0.004621084 | 0.000154325; 61 | SLE GWAS | 79 | Coronary artery disease ||
id: ebi-a-GCST005194 | Inverse variance weighted (fixed effects) | 71
| −0.0174862 | 0.004396787 | 6.97782E−05; 61 | SLE GWAS | 79 | Coronary artery disease ||
id: ebi-a-GCST005194 | Simple mode | 71 | −0.028617859 | 0.010468736 | 0.007925864;
61 | SLE GWAS | 79 | Coronary artery disease || id: ebi-a-GCST005194 | Weighted
mode | 71 | −0.028617859 | 0.00818923 | 0.00082799; 61 | SLE GWAS | 79 | Coronary
artery disease || id: ebi-a-GCST005194 | Weighted mode (NOME) | 71
| −0.028617859 | 0.008896832 | 0.00196489; 61 | SLE GWAS | 79 | Coronary artery disease ||
id: ebi-a-GCST005194 | Simple mode (NOME) | 71
| −0.028617859 | 0.009807086 | 0.00473195; 61 | SLE GWAS | 79 | Coronary artery disease ||
id: ebi-a-GCST005194 | Robust adjusted profile score (RAPS) | 71
| −0.017588109 | 0.004876414 | 0.000310031; 61 | SLE GWAS | 79 | Coronary artery disease
|| id: ebi-a-GCST005194 | Raw | 71 | −0.0174862 | 0.004621084 | 0.000322271; 61 | SLE
GWAS | 79 | Coronary artery disease || id: ebi-a-GCST005194 | Outlier-
corrected | NA NA NA NA; 62 | SLE GWAS | 7 | Coronary artery disease || id: ebi-a-
GCST005194 | Maximum likelihood | 7 | −0.027316568 | 0.021890891 | 0.212085669;
62 | SLE GWAS | 7 | Coronary artery disease || id: ebi-a-GCST005194 | MR Egger | 7
| −0.012587185 | 0.089454229 | 0.893591441; 62 | SLE GWAS | 7 | Coronary artery disease ||
id: ebi-a-GCST005194 | MR Egger | (bootstrap) | 7 | −0.042650389 | 0.046783352 | 0.17;
62 | SLE GWAS | 7 | Coronary artery disease || id: ebi-a-GCST005194 | Simple
median | 7 | −0.041324625 | 0.031910404 | 0.195313181; 62 | SLE GWAS | 7 | Coronary
artery disease || id: ebi-a-GCST005194 | Weighted median | 7
| −0.035326443 | 0.025939722 | 0.173239954; 62 | SLE GWAS | 7 | Coronary artery disease ||
id: ebi-a-GCST005194 | Penalised weighted median | 7
| −0.035781375 | 0.025704945 | 0.163921323; 62 | SLE GWAS | 7 | Coronary artery disease ||
id: ebi-a-GCST005194 | Inverse variance weighted | 7
| −0.023287087 | 0.031159985 | 0.454858653; 62 | SLE GWAS | 7 | Coronary artery disease ||
id: ebi-a-GCST005194 | Inverse variance weighted (multiplicative random effects) | 7
| −0.023287087 | 0.031159985 | 0.454858653; 62 | SLE GWAS | 7 | Coronary artery disease ||
id: ebi-a-GCST005194 | Inverse variance weighted (fixed effects) | 7
| −0.023287087 | 0.020019678 | 0.24474435; 62 | SLE GWAS | 7 | Coronary artery disease ||
id: ebi-a-GCST005194 | Simple mode | 7 | −0.043275246 | 0.034936261 | 0.2617197;
62 | SLE GWAS | 7 | Coronary artery disease || id: ebi-a-GCST005194 | Weighted
mode | 7 | −0.037216084 | 0.024303302 | 0.176571587; 62 | SLE GWAS | 7 | Coronary
artery disease || id: ebi-a-GCST005194 | Weighted mode (NOME) | 7
| −0.037216084 | 0.023687623 | 0.16721154; 62 | SLE GWAS | 7 | Coronary artery disease ||
id: ebi-a-GCST005194 | Simple mode (NOME) | 7
| −0.043275246 | 0.032852943 | 0.235824374; 62 | SLE GWAS | 7 | Coronary artery disease ||
id: ebi-a-GCST005194 | Robust adjusted profile score (RAPS) | 7
| −0.030695037 | 0.029715183 | 0.301615662; 62 | SLE GWAS | 7 | Coronary artery disease ||
id: ebi-a-GCST005194 | Raw | 7 | −0.023287087 | 0.031159985 | 0.483107645; 62 | SLE
GWAS | 7 | Coronary artery disease || id: ebi-a-GCST005194 | Outlier-
corrected | NA | NA | NA | NA; 63 | SLE GWAS | 14 | Coronary artery disease || id: ebi-a-
GCST005194 | Maximum likelihood | 12 | −0.000188753 | 0.01851594 | 0.991866439;
63 | SLE GWAS | 14 | Coronary artery disease || id: ebi-a-GCST005194 | MR
Egger | 12 | 0.014182937 | 0.050694463 | 0.785353361; 63 | SLE GWAS | 14 | Coronary
artery disease || id: ebi-a-GCST005194 | MR Egger | (bootstrap) | 12
| −0.000193509 | 0.050474358 | 0.506; 63 | SLE GWAS | 14 | Coronary artery disease ||
id: ebi-a-GCST005194 | Simple median | 12 | 0.000284061 | 0.025304254 | 0.991043279;
63 | SLE GWAS | 14 | Coronary artery disease || id: ebi-a-GCST005194 | Weighted
median | 12 | 0.006335859 | 0.018790899 | 0.735983471; 63 | SLE GWAS | 14 | Coronary
artery disease || id: ebi-a-GCST005194 | Penalised weighted
median | 12 | 0.006325497 | 0.019861989 | 0.750126573; 63 | SLE GWAS | 14 | Coronary
artery disease || id: ebi-a-GCST005194 | Inverse variance weighted | 12
| −0.000112257 | 0.029910532 | 0.997005483; 63 | SLE GWAS | 14 | Coronary artery disease
|| id: ebi-a-GCST005194 | Inverse variance weighted (multiplicative random effects) | 12
| −0.000112257 | 0.029910532 | 0.997005483; 63 | SLE GWAS | 14 | Coronary artery disease
|| id: ebi-a-GCST005194 | Inverse variance weighted (fixed effects) | 12
| −0.000112257 | 0.01443248 | 0.993794075; 63 | SLE GWAS | 14 | Coronary artery disease ||
id: ebi-a-GCST005194 | Simple mode | 12 | 0.004041475 | 0.023460745 | 0.866357381;
63 | SLE GWAS | 14 | Coronary artery disease || id: ebi-a-GCST005194 | Weighted
mode | 12 | 0.004041475 | 0.01752202 | 0.821819432; 63 | SLE GWAS | 14 | Coronary
artery disease || id: ebi-a-GCST005194 | Weighted mode
(NOME) | 12 | 0.004041475 | 0.016252669 | 0.808201238; 63 | SLE GWAS | 14 | Coronary
artery disease || id: ebi-a-GCST005194 | Simple mode
(NOME) | 12 | 0.004041475 | 0.022050098 | 0.857908273; 63 | SLE GWAS | 14 | Coronary
artery disease || id: ebi-a-GCST005194 | Robust adjusted profile score (RAPS) | 12
| −0.00538804 | 0.029918243 | 0.857080239; 63 | SLE GWAS | 14 | Coronary artery disease ||
id: ebi-a-GCST005194 | Raw | 12 | −0.000112257 | 0.029910532 | 0.997072683; 63 | SLE
GWAS | 14 | Coronary artery disease || id: ebi-a-GCST005194 | Outlier-corrected | 10
| −0.009120889 | 0.020815497 | 0.671585994; 64 | SLE GWAS | 35 | Coronary artery disease
|| id: ebi-a-GCST005194 | Maximum likelihood | 32
| −0.061992457 | 0.006232206 | 2.59607E−23; 64 | SLE GWAS | 35 | Coronary artery disease
|| id: ebi-a-GCST005194 | MR Egger | 32 | −0.046370719 | 0.018447729 | 0.017550031;
64 | SLE GWAS | 35 | Coronary artery disease || id: ebi-a-GCST005194 | MR Egger
(bootstrap) | 32 | −0.071560263 | 0.023398936 | 0.002; 64 | SLE GWAS | 35 | Coronary
artery disease || id: ebi-a-GCST005194 | Simple median | 32
| −0.064075027 | 0.008562014 | 7.22922E−14; 64 | SLE GWAS | 35 | Coronary artery disease
|| id: ebi-a-GCST005194 | Weighted median | 32 | −0.064083621 | 0.008308175 | 1.22585E−14;
64 | SLE GWAS | 35 | Coronary artery disease || id: ebi-a-GCST005194 | Penalised
weighted median | 32 | −0.064084308 | 0.008182437 | 4.8042E−15; 64 | SLE
GWAS | 35 | Coronary artery disease || id: ebi-a-GCST005194 | Inverse variance
weighted | 32 | −0.06103238 | 0.005867262 | 2.42297E−25; 64 | SLE GWAS | 35 | Coronary
artery disease || id: ebi-a-GCST005194 | Inverse variance weighted (multiplicative random
effects) | 32 | −0.06103238 | 0.004510808 | 1.03663E−41; 64 | SLE GWAS | 35 | Coronary
artery disease || id: ebi-a-GCST005194 | Inverse variance weighted (fixed effects) | 32
| −0.06103238 | 0.005867262 | 2.42297E−25; 64 | SLE GWAS | 35 | Coronary artery disease ||
id: ebi-a-GCST005194 | Simple mode | 32 | −0.068062435 | 0.015903521 | 0.000166944;
64 | SLE GWAS | 35 | Coronary artery disease || id: ebi-a-GCST005194 | Weighted
mode | 32 | −0.069228968 | 0.015087755 | 6.96246E−05; 64 | SLE GWAS | 35 | Coronary
artery disease || id: ebi-a-GCST005194 | Weighted mode (NOME) | 32
| −0.069228968 | 0.0147412 | 5.11987E−05; 64 | SLE GWAS | 35 | Coronary artery disease ||
id: ebi-a-GCST005194 | Simple mode (NOME) | 32
| −0.068062435 | 0.014686408 | 6.10829E−05; 64 | SLE GWAS | 35 | Coronary artery disease
|| id: ebi-a-GCST005194 | Robust adjusted profile score (RAPS) | 32
| −0.06275167 | 0.006423604 | 1.53075E−22; 64 | SLE GWAS | 35 | Coronary artery disease ||
id: ebi-a-GCST005194 | Raw | 32 | −0.06103238 | 0.004510808 | 1.50344E−14; 64 | SLE
GWAS | 35 | Coronary artery disease || id: ebi-a-GCST005194 | Outlier-
corrected | NA | NA | NA | NA; 65 | SLE GWAS | 22 | Coronary artery disease || id: ebi-a-
GCST005194 | Maximum likelihood | 18 | 0.044079171 | 0.011562362 | 0.000137681;
65 | SLE GWAS | 22 | Coronary artery disease || id: ebi-a-GCST005194 | MR
Egger | 18 | 0.049077808 | 0.021072154 | 0.033286334; 65 | SLE GWAS | 22 | Coronary
artery disease || id: ebi-a-GCST005194 | MR Egger
(bootstrap) | 18 | 0.044792984 | 0.026699192 | 0.049; 65 | SLE GWAS | 22 | Coronary
artery disease || id: ebi-a-GCST005194 | Simple
median | 18 | 0.045486414 | 0.015855896 | 0.004121127; 65 | SLE GWAS | 22 | Coronary
artery disease || id: ebi-a-GCST005194 | Weighted
median | 18 | 0.045710485 | 0.013875883 | 0.000986883; 65 | SLE GWAS | 22 | Coronary
artery disease || id: ebi-a-GCST005194 | Penalised weighted
median | 18 | 0.045710485 | 0.013968405 | 0.001066311; 65 | SLE GWAS | 22 | Coronary
artery disease || id: ebi-a-GCST005194 | Inverse variance
weighted | 18 | 0.044328019 | 0.011295497 | 8.69472E−05; 65 | SLE GWAS | 22 | Coronary
artery disease || id: ebi-a-GCST005194 | Inverse variance weighted (multiplicative random
effects) | 18 | 0.044328019 | 0.003994578 | 1.29659E−28; 65 | SLE GWAS | 22 | Coronary
artery disease || id: ebi-a-GCST005194 | Inverse variance weighted (fixed
effects) | 18 | 0.044328019 | 0.011295497 | 8.69472E−05; 65 | SLE GWAS | 22 | Coronary
artery disease || id: ebi-a-GCST005194 | Simple
mode | 18 | 0.045965112 | 0.017072136 | 0.015417455; 65 | SLE GWAS | 22 | Coronary
artery disease || id: ebi-a-GCST005194 | Weighted
mode | 18 | 0.045965112 | 0.016660757 | 0.013415701; 65 | SLE GWAS | 22 | Coronary
artery disease || id: ebi-a-GCST005194 | Weighted mode
(NOME) | 18 | 0.045965112 | 0.015938521 | 0.010308161; 65 | SLE GWAS | 22 | Coronary
artery disease || id: ebi-a-GCST005194 | Simple mode
(NOME) | 18 | 0.045965112 | 0.016199029 | 0.011369834; 65 | SLE GWAS | 22 | Coronary
artery disease || id: ebi-a-GCST005194 | Robust adjusted profile score
(RAPS) | 18 | 0.044594428 | 0.011984108 | 0.000198333; 65 | SLE GWAS | 22 | Coronary
artery disease || id: ebi-a-GCST005194 | Raw | 18 | 0.044328019 | 0.003994578 | 3.29783E−09;
65 | SLE GWAS | 22 | Coronary artery disease || id: ebi-a-GCST005194 | Outlier-
corrected | NA | NA | NA | NA; 66 | SLE GWAS | 7 | Coronary artery disease || id: ebi-a-
GCST005194 | Maximum likelihood | 6 | −0.216791606 | 0.077542498 | 0.005177496;
66 | SLE GWAS | 7 | Coronary artery disease || id: ebi-a-GCST005194 | MR Egger | 6
| −0.212792422 | 0.112756233 | 0.132178754; 66 | SLE GWAS | 7 | Coronary artery disease ||
id: ebi-a-GCST005194 | MR Egger | (bootstrap) | 6 | −0.068134279 | 0.172003458 | 0.337;
66 | SLE GWAS | 7 | Coronary artery disease || id: ebi-a-GCST005194 | Simple
median | 6 | −0.213422965 | 0.104541205 | 0.041199165; 66 | SLE GWAS | 7 | Coronary
artery disease || id: ebi-a-GCST005194 | Weighted median | 6
| −0.15275753 | 0.291924261 | 0.600780842; 66 | SLE GWAS | 7 | Coronary artery disease ||
id: ebi-a-GCST005194 | Penalised weighted median | 6
| −0.15275753 | 0.115550436 | 0.186168574; 66 | SLE GWAS | 7 | Coronary artery disease ||
id: ebi-a-GCST005194 | Inverse variance weighted | 6
| −0.163102892 | 0.05993631 | 0.006503159; 66 | SLE GWAS | 7 | Coronary artery disease ||
id: ebi-a-GCST005194 | Inverse variance weighted (multiplicative random effects) | 6
| −0.163102892 | 0.05993631 | 0.006503159; 66 | SLE GWAS | 7 | Coronary artery disease ||
id: ebi-a-GCST005194 | Inverse variance weighted (fixed effects) | 6
| −0.163102892 | 0.045131858 | 0.000301603; 66 | SLE GWAS | 7 | Coronary artery disease
id: ebi-a-GCST005194 | Simple mode | 6 | −0.218011995 | 0.104822742 | 0.092083471;
66 | SLE GWAS | 7 | Coronary artery disease || id: ebi-a-GCST005194 | Weighted
mode | 6 | −0.156687472 | 0.072944517 | 0.084454461; 66 | SLE GWAS | 7 | Coronary
artery disease || id: ebi-a-GCST005194 | Weighted mode (NOME) | 6
| −0.169463414 | 0.053943299 | 0.025622725; 66 | SLE GWAS | 7 | Coronary artery disease ||
id: ebi-a-GCST005194 | Simple mode (NOME) | 6
| −0.218011995 | 0.080246901 | 0.041936365; 66 | SLE GWAS | 7 | Coronary artery disease ||
id: ebi-a-GCST005194 | Robust adjusted profile score (RAPS) | 6
| −0.211931777 | 0.094459982 | 0.024857221; 66 | SLE GWAS | 7 | Coronary artery disease ||
id: ebi-a-GCST005194 | Raw | 6 | −0.163102892 | 0.05993631 | 0.04171176; 66 | SLE
GWAS | 7 | Coronary artery disease || id: ebi-a-GCST005194 | Outlier-
corrected | NA | NA | NA | NA; 67 | SLE GWAS | 17 | Coronary artery disease || id: ebi-a-
GCST005194 | Maximum likelihood | 14 | 0.106664813 | 0.013086265 | 3.61231E−16;
67 | SLE GWAS | 17 | Coronary artery disease || id: ebi-a-GCST005194 | MR
Egger | 14 | 0.007723285 | 0.050607703 | 0.881241169; 67 | SLE GWAS | 17 | Coronary
artery disease || id: ebi-a-GCST005194 | MR Egger
(bootstrap) | 14 | 0.144579802 | 0.040636912 | 0; 67 | SLE GWAS | 17 | Coronary artery
disease || id: ebi-a-GCST005194 | Simple
median | 14 | 0.026309865 | 0.026001579 | 0.311606692; 67 | SLE GWAS | 17 | Coronary
artery disease || id: ebi-a-GCST005194 | Weighted
median | 14 | 0.015020317 | 0.013419707 | 0.263023668; 67 | SLE GWAS | 17 | Coronary
artery disease || id: ebi-a-GCST005194 | Penalised weighted
median | 14 | 0.01487686 | 0.014170589 | 0.293791383; 67 | SLE GWAS | 17 | Coronary
artery disease || id: ebi-a-GCST005194 | Inverse variance
weighted | 14 | 0.073253914 | 0.034466081 | 0.033553966; 67 | SLE GWAS | 17 | Coronary
artery disease || id: ebi-a-GCST005194 | Inverse variance weighted (multiplicative random
effects) | 14 | 0.073253914 | 0.034466081 | 0.033553966; 67 | SLE GWAS | 17 | Coronary
artery disease || id: ebi-a-GCST005194 | Inverse variance weighted (fixed
effects) | 14 | 0.073253914 | 0.01015612 | 5.48187E−13; 67 | SLE GWAS | 17 | Coronary
artery disease || id: ebi-a-GCST005194 | Simple
mode | 14 | 0.019109804 | 0.015855822 | 0.249598164; 67 | SLE GWAS | 17 | Coronary
artery disease || id: ebi-a-GCST005194 | Weighted
mode | 14 | 0.019109804 | 0.011798681 | 0.129299244; 67 | SLE GWAS | 17 | Coronary
artery disease || id: ebi-a-GCST005194 | Weighted mode
(NOME) | 14 | 0.019109804 | 0.011916609 | 0.132804586; 67 | SLE GWAS | 17 | Coronary
artery disease || id: ebi-a-GCST005194 | Simple mode
(NOME) | 14 | 0.019109804 | 0.016880087 | 0.278046301; 67 | SLE GWAS | 17 | Coronary
artery disease || id: ebi-a-GCST005194 | Robust adjusted profile score
(RAPS) | 14 | 0.033604536 | 0.022280645 | 0.131493391; 67 | SLE GWAS | 17 | Coronary
artery disease || id: ebi-a-
GCST005194 | Raw | 14 | 0.073253914 | 0.034466081 | 0.053294929; 67 | SLE
GWAS | 17 | Coronary artery disease || id: ebi-a-GCST005194 | Outlier-
corrected | 11 | 0.025411991 | 0.015194152 | 0.125376267;

TABLE 3G
MR results (FIG. 13A). Listed by: Cluster | Exposure GWAS | Outcome | MR
method | # SNPs | beta | SE | pval.

All SNPs | SLE GWAS | Coronary artery disease ∥ id: ebi-a-GCST005194 | Maximum
likelihood | 260 | 0.00391388 | 0.00413889 | 0.34433461; All SNPs | SLE
GWAS | Coronary artery disease ∥ id: ebi-a-GCST005194 | MR
Egger | 260 | 0.00327573 | 0.00732222 | 0.6549847; All SNPs | SLE GWAS | Coronary
artery disease ∥ id: ebi-a-GCST005194 | MR Egger
(bootstrap) | 260 | 0.00159623 | 0.00689207 | 0.41; All SNPs | SLE GWAS | Coronary
artery disease ∥ id: ebi-a-GCST005194 | Simple
median | 260 | 0.00727793 | 0.00848138 | 0.39083375; All SNPs | SLE GWAS | Coronary
artery disease ∥ id: ebi-a-GCST005194 | Weighted
median | 260 | 0.00460796 | 0.00664421 | 0.48797697; All SNPs | SLE GWAS | Coronary
artery disease ∥ id: ebi-a-GCST005194 | Penalised weighted
median | 260 | 0.00496983 | 0.00650418 | 0.44480903; All SNPs | SLE GWAS | Coronary
artery disease ∥ id: ebi-a-GCST005194 | Inverse variance
weighted | 260 | 0.00364131 | 0.00510378 | 0.47556552; All SNPs | SLE
GWAS | Coronary artery disease ∥ id: ebi-a-GCST005194 | Inverse variance weighted
(multiplicative random effects) | 260 | 0.00364131 | 0.00510378 | 0.47556552; All
SNPs | SLE GWAS | Coronary artery disease ∥ id: ebi-a-GCST005194 | Inverse variance
weighted (fixed effects) | 260 | 0.00364131 | 0.0037465 | 0.33108924; All SNPs | SLE
GWAS | Coronary artery disease ∥ id: ebi-a-GCST005194 | Simple
mode | 260 | 0.02749447 | 0.01455907 | 0.06007934; All SNPs | SLE GWAS | Coronary
artery disease ∥ id: ebi-a-GCST005194 | Weighted
mode | 260 | 0.00537483 | 0.00941918 | 0.56874827; All SNPs | SLE GWAS | Coronary
artery disease ∥ id: ebi-a-GCST005194 | Weighted mode
(NOME) | 260 | 0.00537483 | 0.00593879 | 0.36628681; All SNPs | SLE GWAS | Coronary
artery disease ∥ id: ebi-a-GCST005194 | Simple mode
(NOME) | 260 | 0.02749447 | 0.012022 | 0.02300212; All SNPs | SLE GWAS | Coronary
artery disease ∥ id: ebi-a-GCST005194 | Robust adjusted profile score
(RAPS) | 260 | 0.00491233 | 0.00549519 | 0.37135801; All SNPs | SLE GWAS | Coronary
artery disease ∥ id: ebi-a-GCST005194 | MR-PRESSO
(raw) | 260 | 0.00364131 | 0.00510378 | 0.47620789; All SNPs | SLE GWAS | Coronary
artery disease ∥ id: ebi-a-GCST005194 | MR-PRESSO (outlier-
corrected) | 255 | 0.00209381 | 0.00482779 | 0.6648747; Positive-causal Tier 1 and Tier
2 | SLE GWAS | Coronary artery disease ∥ id: ebi-a-GCST005194 | Maximum
likelihood | 164 | 0.01276242 | 0.00467853 | 0.0063745; Positive-causal Tier 1 and Tier
2 | SLE GWAS | Coronary artery disease ∥ id: ebi-a-GCST005194 | MR
Egger | 164 | 0.0171299 | 0.00868691 | 0.05032141; Positive-causal Tier 1 and Tier 2 | SLE
GWAS | Coronary artery disease ∥ id: ebi-a-GCST005194 | MR Egger
(bootstrap) | 164 | 0.00710273 | 0.00767102 | 0.176; Positive-causal Tier 1 and Tier 2 | SLE
GWAS | Coronary artery disease ∥ id: ebi-a-GCST005194 | Simple
median | 164 | 0.02141378 | 0.00950742 | 0.02430191; Positive-causal Tier 1 and Tier
2 | SLE GWAS | Coronary artery disease ∥ id: ebi-a-GCST005194 | Weighted
median | 164 | 0.01802509| 0.00743329 | 0.01531206; Positive-causal Tier 1 and Tier
2 | SLE GWAS | Coronary artery disease ∥ id: ebi-a-GCST005194 | Penalised weighted
median | 164 | 0.01816446 | 0.00717028 | 0.01129946; Positive-causal Tier 1 and Tier
2 | SLE GWAS | Coronary artery disease ∥ id: ebi-a-GCST005194 | Inverse variance
weighted | 164 | 0.01236131 | 0.00595914 | 0.03804731; Positive-causal Tier 1 and Tier
2 | SLE GWAS | Coronary artery disease ∥ id: ebi-a-GCST005194 | Inverse variance
weighted (multiplicative random effects) | 164 | 0.01236131 | 0.00595914 | 0.03804731;
Positive-causal Tier 1 and Tier 2 | SLE GWAS | Coronary artery disease ∥ id: ebi-a-
GCST005194 | Inverse variance weighted (fixed
effects) | 164 | 0.01236131 | 0.00428945 | 0.00395421; Positive-causal Tier 1 and Tier
2 | SLE GWAS | Coronary artery disease ∥ id: ebi-a-GCST005194 | Simple
mode | 164 | 0.03396991 | 0.01463561 | 0.02152286; Positive-causal Tier 1 and Tier
2 | SLE GWAS | Coronary artery disease ∥ id: ebi-a-GCST005194 | Weighted
mode | 164 | 0.01810676 | 0.00957599 | 0.06041865; Positive-causal Tier 1 and Tier
2 | SLE GWAS | Coronary artery disease ∥ id: ebi-a-GCST005194 | Weighted mode
(NOME) | 164 | 0.01810676 | 0.0063545 | 0.00494516; Positive-causal Tier 1 and Tier
2 | SLE GWAS | Coronary artery disease ∥ id: ebi-a-GCST005194 | Simple mode
(NOME) | 164 | 0.03396991 | 0.01392296 | 0.01576514; Positive-causal Tier 1 and Tier
2 | SLE GWAS | Coronary artery disease ∥ id: ebi-a-GCST005194 | Robust adjusted profile
score (RAPS) | 164 | 0.01405697 | 0.00640752 | 0.02824817; Positive-causal Tier 1 and
Tier 2 | SLE GWAS | Coronary artery disease ∥ id: ebi-a-GCST005194 | MR-PRESSO
(raw) | 164 | 0.01236131 | 0.00595914 | 0.03961934; Positive-causal Tier 1 and Tier
2 | SLE GWAS | Coronary artery disease ∥ id: ebi-a-GCST005194 | MR-PRESSO (outlier-
corrected) | 161 | 0.00986883 | 0.00562089 | 0.08104589; Positive-causal Tier 1 | SLE
GWAS | Coronary artery disease ∥ id: ebi-a-GCST005194 | Maximum
likelihood | 133 | 0.01308005 | 0.00505562 | 0.00967516; Positive-causal Tier 1 | SLE
GWAS | Coronary artery disease ∥ id: ebi-a-GCST005194 | MR
Egger | 133 | 0.0147978 | 0.00954029 | 0.12329396; Positive-causal Tier 1 | SLE
GWAS | Coronary artery disease ∥ id: ebi-a-GCST005194 | MR Egger
(bootstrap) | 133 | 0.01006575 | 0.00860565 | 0.125; Positive-causal Tier 1 | SLE
GWAS | Coronary artery disease ∥ id: ebi-a-GCST005194 | Simple
median | 133 | 0.0206695 | 0.01062334 | 0.05169446; Positive-causal Tier 1 | SLE
GWAS | Coronary artery disease ∥ id: ebi-a-GCST005194 | Weighted
median | 133 | 0.01813236 | 0.00739774 | 0.01424337; Positive-causal Tier 1 | SLE
GWAS | Coronary artery disease ∥ id: ebi-a-GCST005194 | Penalised weighted
median | 133 | 0.01813963 | 0.00745942 | 0.01502511; Positive-causal Tier 1 | SLE
GWAS | Coronary artery disease ∥ id: ebi-a-GCST005194 | Inverse variance
weighted | 133 | 0.01203144 | 0.00673853 | 0.07418496; Positive-causal Tier 1 | SLE
GWAS | Coronary artery disease ∥ id: ebi-a-GCST005194 | Inverse variance weighted
(multiplicative random effects) | 133 | 0.01203144 | 0.00673853 | 0.07418496; Positive-
causal Tier 1 | SLE GWAS | Coronary artery disease ∥ id: ebi-a-GCST005194 | Inverse
variance weighted (fixed effects) | 133 | 0.01203144 | 0.00459831 | 0.0088838; Positive-
causal Tier 1 | SLE GWAS | Coronary artery disease ∥ id: ebi-a-GCST005194 | Simple
mode | 133 | 0.02517371 | 0.01613804 | 0.12117816; Positive-causal Tier 1 | SLE
GWAS | Coronary artery disease ∥ id: ebi-a-GCST005194 | Weighted
mode | 133 | 0.01852232 | 0.01198381 | 0.12459265; Positive-causal Tier 1 | SLE
GWAS | Coronary artery disease ∥ id: ebi-a-GCST005194 | Weighted mode
(NOME) | 133 | 0.01852232 | 0.00637512 | 0.00430215; Positive-causal Tier 1 | SLE
GWAS | Coronary artery disease ∥ id: ebi-a-GCST005194 | Simple mode
(NOME) | 133 | 0.02517371 | 0.01347593 | 0.06397226; Positive-causal Tier 1 | SLE
GWAS | Coronary artery disease ∥ id: ebi-a-GCST005194 | Robust adjusted profile score
(RAPS) | 133 | 0.0128258 | 0.00706773 | 0.06957043; Positive-causal Tier 1 | SLE
GWAS | Coronary artery disease ∥ id: ebi-a-GCST005194 | MR-PRESSO
(raw) | 133 | 0.01203144 | 0.00673853 | 0.07648124; Positive-causal Tier 1 | SLE
GWAS | Coronary artery disease ∥ id: ebi-a-GCST005194 | MR-PRESSO (outlier-
corrected) | 129 | 0.00894607 | 0.00619578 | 0.1512108; Positive-causal MR-IVW p-
value < 0.00075 | SLE GWAS | Coronary artery disease ∥ id: ebi-a-GCST005194 | Maximum
likelihood | 117 | 0.015379 | 0.00583127 | 8.36E−03; Positive-causal MR-IVW p-
value < 0.00075 | SLE GWAS | Coronary artery disease ∥ id: ebi-a-GCST005194 | MR
Egger | 117 | 0.01855383 | 0.01037295 | 0.0763002; Positive-causal MR-IVW p-
value < 0.00075 | SLE GWAS | Coronary artery disease ∥ id: ebi-a-GCST005194 | MR Egger
(bootstrap) | 117 | 0.01331575 | 0.0096114 | 0.086; Positive-causal MR-IVW p-
value < 0.00075 | SLE GWAS | Coronary artery disease ∥ id: ebi-a-GCST005194 | Simple
median | 117 | 0.01856629 | 0.01143091 | 0.10432935; Positive-causal MR-IVW p-
value < 0.00075 | SLE GWAS | Coronary artery disease ∥ id: ebi-a-GCST005194 | Weighted
median | 117 | 0.0221029 | 0.00871229 | 0.01118136; Positive-causal MR-IVW p-
value < 0.00075 | SLE GWAS | Coronary artery disease ∥ id: ebi-a-GCST005194 | Penalised
weighted median | 117 | 0.02210599 | 0.00888328 | 0.01282856; Positive-causal MR-IVW
p-value < 0.00075 | SLE GWAS | Coronary artery disease ∥ id: ebi-a-GCST005194 | Inverse
variance weighted | 117 | 0.01411765 | 0.00717875 | 4.92E−02; Positive-causal MR-IVW p-
value < 0.00075 | SLE GWAS | Coronary artery disease ∥ id: ebi-a-GCST005194 | Inverse
variance weighted (multiplicative random effects) | 117 | 0.01411765 | 0.00717875 | 4.92E−
02; Positive-causal MR-IVW p-value < 0.00075 | SLE GWAS | Coronary artery disease ∥
id: ebi-a-GCST005194 | Inverse variance weighted (fixed
effects) | 117 | 0.01411765 | 0.00525151 |7.18E−03; Positive-causal MR-IVW p-
value < 0.00075 | SLE GWAS | Coronary artery disease ∥ id: ebi-a-GCST005194 | Simple
mode | 117 | 0.0265057 | 0.01954883 | 0.17777373; Positive-causal MR-IVW p-
value < 0.00075 | SLE GWAS | Coronary artery disease ∥ id: ebi-a-GCST005194 | Weighted
mode | 117 | 0.01968787 | 0.01404779 | 0.16373692; Positive-causal MR-IVW p-
value < 0.00075 | SLE GWAS | Coronary artery disease ∥ id: ebi-a-GCST005194 | Weighted
mode (NOME) | 117 | 0.01968787 | 0.00778374 | 0.01276933; Positive-causal MR-IVW p-
value < 0.00075 | SLE GWAS | Coronary artery disease ∥ id: ebi-a-GCST005194 | Simple
mode (NOME) | 117 | 0.0265057 | 0.01552385 | 0.0904196; Positive-causal MR-IVW p-
value < 0.00075 | SLE GWAS | Coronary artery disease ∥ id: ebi-a-GCST005194 | Robust
adjusted profile score (RAPS) | 117 | 0.01550812 | 0.00798438 | 5.21E−02; Positive-causal
MR-IVW p-value < 0.00075 | SLE GWAS | Coronary artery disease ∥ id: ebi-a-
GCST005194 | MR-PRESSO (raw) | 117 | 0.01411765 | 0.00717875 | 0.05161886;
Positive-casual MR-IVW p-value < 0.00075 | SLE GWAS | Coronary artery disease ∥ id: ebi-
a-GCST005194 | MR-PRESSO (outlier-
corrected) | 116 | 0.01224365 | 0.00704022 | 0.08469293; Negative-causal Tier 1 and Tier
2 | SLE GWAS | Coronary artery disease ∥ id: ebi-a-GCST005194 | Maximum
likelihood | 62 | −0.0294783 | 0.00848042 | 0.00050888; Negative-causal Tier 1 and Tier
2 | SLE GWAS | Coronary artery disease ∥ id: ebi-a-GCST005194 | MR Egger | 62 | −0.0177862 |
0.01652333 | 0.28604349; Negative-causal Tier 1 and Tier 2 | SLE
GWAS | Coronary artery disease ∥ id: ebi-a-GCST005194 | MR Egger (bootstrap) | 62 | −0.0034443 |
0.0147415 | 0.401; Negative-causal Tier 1 and Tier 2 | SLE GWAS | Coronary
artery disease | id: ebi-a-GCST005194 | Simple median | 62 | −0.02054 |
0.01666381 | 0.21772042; Negative-causal Tier 1 and Tier 2 | SLE
GWAS | Coronary artery disease ∥ id: ebi-a-GCST005194 | Weighted median | 62| −0.0056944 |
0.01210317 | 0.63800864; Negative-causal Tier 1 and Tier 2 | SLE
GWAS | Coronary artery disease ∥ id: ebi-a-GCST005194 | Penalised weighted
median | 62 | −0.00544 | 0.01241635 | 0.66128956; Negative-causal Tier 1 and Tier 2 | SLE
GWAS | Coronary artery disease ∥ id: ebi-a-GCST005194 | Inverse variance
weighted | 62 | −0.026251 | 0.01112607 | 0.01830385; Negative-causal Tier 1 and Tier
2 | SLE GWAS | Coronary artery disease ∥ id: ebi-a-GCST005194 | Inverse variance
weighted (multiplicative random effects) | 62 | −0.026251 | 0.01112607 | 0.01830385;
Negative-causal Tier 1 and Tier 2 | SLE GWAS | Coronary artery disease ∥ id: ebi-a-
GCST005194 | Inverse variance weighted (fixed effects) | 62 | −0.026251 |
0.00760399 | 0.0005559; Negative-causal Tier 1 and Tier 2 | SLE
GWAS | Coronary artery disease ∥ id: ebi-a-GCST005194 | Simple mode | 62 | −0.010628 |
0.02045318 | 0.60520358; Negative-causal Tier 1 and Tier 2 | SLE
GWAS | Coronary artery disease ∥ id: ebi-a-GCST005194 | Weighted mode | 62 | −0.006857 |
0.01124272 | 0.54418695; Negative-causal Tier 1 and Tier 2 | SLE
GWAS | Coronary artery disease ∥ id: ebi-a-GCST005194 | Weighted mode (NOME) | 62 | −0.006857 |
0.0110847 | 0.53848002; Negative-causal Tier 1 and Tier 2 | SLE
GWAS | Coronary artery disease ∥ id: ebi-a-GCST005194 | Simple mode (NOME) | 62 | −0.010628 |
0.02441606 | 0.66488907; Negative-causal Tier 1 and Tier 2 | SLE
GWAS | Coronary artery disease ∥ id: ebi-a-GCST005194 | Robust adjusted profile score
(RAPS) | 62 | −0.0329958 | 0.01231037 | 0.00735507; Negative-causal Tier 1 and Tier
2 | SLE GWAS | Coronary artery disease ∥ id: ebi-a-GCST005194 | MR-PRESSO
(raw) | 62 | −0.026251 | 0.01112607 | 0.02152078; Negative-causal Tier 1 and Tier 2 | SLE
GWAS | Coronary artery disease ∥ id: ebi-a-GCST005194 | MR-PRESSO (outlier-
corrected) | | | |; Negative-causal Tier 1 | SLE GWAS | Coronary artery disease ∥ id: ebi-
a-GCST005194 | Maximum likelihood | 41 | −0.0431742 | 0.01159112 | 0.0001955;
Negative-causal Tier 1 | SLE GWAS | Coronary artery disease ∥ id: ebi-a-
GCST005194 | MR Egger | 41 | −0.0408695 | 0.02236202 | 0.07526375; Negative-causal
Tier 1 | SLE GWAS | Coronary artery disease ∥ id: ebi-a-GCST005194 | MR Egger
(bootstrap) | 41 | −0.0378234 | 0.02385499 | 0.054; Negative-causal Tier 1 | SLE
GWAS | Coronary artery disease ∥ id: ebi-a-GCST005194 | Simple median | 41 | −0.0251154 |
0.02426454 | 0.30063732; Negative-causal Tier 1 | SLE GWAS | Coronary
artery disease | id: ebi-a-GCST005194 | Weighted median | 41
0.0214734 | 0.01833815 | 0.24161066; Negative-causal Tier 1 | SLE GWAS | Coronary
artery disease | id: ebi-a-GCST005194 | Penalised weighted median | 41 | −0.020521 |
0.0176714 | 0.24553793; Negative-causal Tier 1 | SLE GWAS | Coronary artery
disease | id: ebi-a-GCST005194 | Inverse variance weighted | 41 | −0.039399 |
0.01484755 | 0.00796454; Negative-causal Tier 1 | SLE GWAS | Coronary
artery disease | id: ebi-a-GCST005194 | Inverse variance weighted (multiplicative random
effects) | 41 | −0.039399 | 0.01484755 | 0.00796454; Negative-causal Tier 1 | SLE
GWAS | Coronary artery disease ∥ id: ebi-a-GCST005194 | Inverse variance weighted
(fixed effects) | 41 | −0.039399 | 0.01022724 | 0.00011699; Negative-causal Tier 1 | SLE
GWAS | Coronary artery disease ∥ id: ebi-a-GCST005194 | Simple mode | 41 | −0.0136888 |
0.02882848 | 0.63748625; Negative-causal Tier 1 | SLE GWAS | Coronary
artery disease | id: ebi-a-GCST005194 | Weighted mode | 41 | −0.0240864 |
0.01349456 | 0.08186313; Negative-causal Tier 1 | SLE GWAS | Coronary
artery disease | id: ebi-a-GCST005194 | Weighted mode (NOME) | 41 | −0.0310182 |
0.01543743 | 0.05128818; Negative-causal Tier 1 | SLE GWAS | Coronary
artery disease | id: ebi-a-GCST005194 | Simple mode (NOME) | 41 | −0.0136888 |
0.03071507 | 0.65823824; Negative-causal Tier 1 | SLE GWAS | Coronary
artery disease | id: ebi-a-GCST005194 | Robust adjusted profile score (RAPS) | 41 | −0.0438782 |
0.01593987 | 0.00591006; Negative-causal Tier 1 | SLE GWAS | Coronary
artery disease | id: ebi-a-GCST005194 | MR-PRESSO (raw) | 41 | −0.039399 |
0.01484755 | 0.01137031; Negative-causal Tier 1 | SLE GWAS | Coronary
artery disease | id: ebi-a-GCST005194 | MR-PRESSO (outlier-corrected) | 40 | −0.0404701 |
0.01410666 | 0.00661751; Negative-causal MR-IVW p-value < 0.00075 | SLE
GWAS | Coronary artery disease ∥ id: ebi-a-GCST005194 | Maximum likelihood | 49 | −0.0376213 |
0.0101698 | 0.00021618; Negative-causal MR-IVW p-value < 0.00075 | SLE
GWAS | Coronary artery disease ∥ id: ebi-a-GCST005194 | MR Egger | 49 | −0.0307312 |
0.02009799 | 0.13295081; Negative-causal MR-IVW p-value < 0.00075 | SLE
GWAS | Coronary artery disease ∥ id: ebi-a-GCST005194 | MR Egger (bootstrap) | 49 | −0.0289513 |
0.02062338 | 0.086; Negative-causal MR-IVW p-value < 0.00075 | SLE
GWAS |Coronary artery disease ∥ id: ebi-a-GCST005194 | Simple median | 49 | −0.0201993 |
0.01817733 | 0.26646596; Negative-causal MR-IVW p-value < 0.00075 | SLE
GWAS |Coronary artery disease ∥ id: ebi-a-GCST005194 | Weighted median | 49 | −0.0086248 |
0.01517963 | 0.56990965; Negative-causal MR-IVW p-value < 0.00075 | SLE |
GWAS | Coronary artery disease ∥ id: ebi-a-GCST005194 | Penalised weighted
median | 49 | −0.0079257 | 0.01477298 | 0.59161249; Negative-causal MR-IVW p-
value < 0.00075 | SLE GWAS | Coronary artery disease ∥ id: ebi-a-GCST005194 | Inverse
variance weighted | 49 |−0.0341934 | 0.01274725 | 0.00730933; Negative-causal MR-IVW
p-value < 0.00075 | SLE GWAS | Coronary artery disease ∥ id: ebi-a-GCST005194 | Inverse
variance weighted (multiplicative random effects) | 49 | −0.0341934 |
0.01274725 | 0.00730933; Negative-causal MR-IVW p-value < 0.00075 | SLE
GWAS |Coronary artery disease ∥ id: ebi-a-GCST005194 | Inverse variance weighted
(fixed effects) | 49 | −0.0341934 | 0.0090623 | 0.0001612; Negative-causal MR-IVW p-
value < 0.00075 | SLE GWAS | Coronary artery disease ∥ id: ebi-a-GCST005194 | Simple
mode | 49 | −0.0110106 | 0.02306041 | 0.63519538; Negative-causal MR-IVW p-
value < 0.00075 | SLE GWAS | Coronary artery disease ∥ id: ebi-a-GCST005194 | Weighted
mode | 49 | −0.0143141 | 0.01286279 | 0.2713248; Negative-causal MR-IVW p-
value < 0.00075 | SLE GWAS | Coronary artery disease ∥ id: ebi-a-GCST005194 | Weighted
mode (NOME) | 49 | −0.0176175 | 0.01382604 | 0.20871799; Negative-causal MR-IVW p-
value < 0.00075 | SLE GWAS | Coronary artery disease ∥ id: ebi-a-GCST005194 | Simple
mode (NOME) | 49 | −0.0110106 | 0.02466241 | 0.65727725; Negative-causal MR-IVW p-
value < 0.00075 | SLE GWAS | Coronary artery disease ∥ id: ebi-a-GCST005194 | Robust
adjusted profile score (RAPS) | 49 | −0.0360293 | 0.01374009 | 8.74E−03; Negative-causal
MR-IVW p-value < 0.00075 | SLE GWAS | Coronary artery disease ∥ id: ebi-a-
GCST005194 | MR-PRESSO (raw) | 49 | −0.0341934 | 0.01274725 | 0.0099946; Negative-
causal MR-IVW p-value < 0.00075 | SLE GWAS | Coronary artery disease ∥ id: ebi-a-
GCST005194 | MR-PRESSO (outlier-corrected) | | | |; Insignificant | SLE
GWAS | Coronary artery disease ∥ id: ebi-a-GCST005194 | Maximum
likelihood | 134 | 0.00982146 | 0.00524729 | 0.06124505; Insignificant | SLE
GWAS | Coronary artery disease ∥ id: ebi-a-GCST005194 | MR
Egger | 134 | 0.0088871 | 0.01021107 | 0.38569463; Insignificant | SLE GWAS | Coronary
artery disease | id: ebi-a-GCST005194 | MR Egger
(bootstrap) | 134 | 0.01252031 | 0.01006407 | 0.111; Insignificant | SLE GWAS | Coronary
artery disease | id: ebi-a-GCST005194 | Simple
median | 134 | 0.00711385 | 0.01051951 | 0.49888022; Insignificant | SLE
GWAS | Coronary artery disease ∥ id: ebi-a-GCST005194 | Weighted
median | 134 | 0.00620539 | 0.00822329 | 0.45048185; Insignificant | SLE
GWAS | Coronary artery disease ∥ id: ebi-a-GCST005194 | Penalised weighted
median | 134 | 0.00613414 | 0.00817826 | 0.45322156; Insignificant | SLE
GWAS | Coronary artery disease ∥ id: ebi-a-GCST005194 | Inverse variance
weighted | 134 | | 0.00881587 | 0.00473924 | 0.06285931; Insignificant | SLE
GWAS |Coronary artery disease ∥ id: ebi-a-GCST005194 | Inverse variance weighted
(multiplicative random effects) | 134 | 0.00881587 | 0.00710064 | 0.21439903;
Insignificant | SLE GWAS | Coronary artery disease ∥ id: ebi-a-GCST005194 | Inverse
variance weighted (fixed effects) | 134 | 0.00881587 | 0.00473924 | 0.06285931;
Insignificant | SLE GWAS | Coronary artery disease ∥ id: ebi-a-GCST005194 | Simple
mode | 134 | 0.03191362 | 0.01667894 | 0.05784476; Insignificant SLE GWAS | Coronary
artery disease | id: ebi-a-GCST005194 | Weighted
mode | 134 | 0.01068318 | 0.01162744 | 0.35986877; Insignificant SLE GWAS | Coronary
artery disease | id: ebi-a-GCST005194 Weighted mode
(NOME) | 134 | 0.01068318 | 0.00699667 | 0.129162; Insignificant | SLE
GWAS | Coronary artery disease ∥ id: ebi-a-GCST005194 | Simple mode
(NOME) | 134 | 0.03191362 | 0.01553496 | 0.04190417; Insignificant | SLE
GWAS | Coronary artery disease ∥ id: ebi-a-GCST005194 | Robust adjusted profile score
(RAPS) | 134 | 0.00785148 | 0.00724744 | 0.2786553; Insignificant | SLE
GWAS | Coronary artery disease ∥ id: ebi-a-GCST005194 | MR-PRESSO
(raw) | 134 | 0.00881587 | 0.00710064 | 0.21658416; Insignificant | SLE GWAS | Coronary
artery disease | id: ebi-a-GCST005194 | MR-PRESSO (outlier-
corrected) | 131 | 0.00352178 | 0.00672363 | 0.60131562;

TABLE 3H
MR results (FIG. 13B). Listed by: Cluster | Exposure GWAS | Outcome | MR
method | # SNPs | beta | SE | pval.

All SNPs | SLE_Immunochip | Coronary artery disease ∥ id: ebi-a-
GCST005194 | Maximum likelihood | 250 | 0.01324279 | 0.00367047 | 0.00030865; All
SNPs | SLE_Immunochip | Coronary artery disease ∥ id: ebi-a-GCST005194 | MR
Egger | 250 | 0.00731712 | 0.0101945 | 0.47358597; All
SNPs | SLE_Immunochip | Coronary artery disease ∥ id: ebi-a-GCST005194 | MR Egger
(bootstrap) | 250 | 0.01055473 | 0.00817715 | 0.104; All
SNPs | SLE_Immunochip | Coronary artery disease ∥ id: ebi-a-GCST005194 | Simple
median | 250 | 0.01016148 | 0.00598228 | 0.08939503; All
SNPs | SLE_Immunochip | Coronary artery disease ∥ id: ebi-a-GCST005194 | Weighted
median | 250 | 0.00977587 | 0.0062102 | 0.1154496; All
SNPs | SLE_Immunochip | Coronary artery disease ∥ id: ebi-a-GCST005194 | Penalised
weighted median | 250 | 0.00974897 | 0.00614288 | 0.11250479; All
SNPs | SLE_Immunochip | Coronary artery disease ∥ id: ebi-a-GCST005194 | Inverse
variance weighted | 250 | 0.01266393 | 0.00461884 | 0.00611038; All
SNPs | SLE_Immunochip | Coronary artery disease ∥ id: ebi-a-GCST005194 | Inverse
variance weighted (multiplicative random
effects) | 250 | 0.01266393 | 0.00461884 | 0.00611038; All
SNPs | SLE_Immunochip | Coronary artery disease ∥ id: ebi-a-GCST005194 | Inverse
variance weighted (fixed effects) | 250 | 0.01266393 | 0.00346566 | 0.00025807; All
SNPs | SLE_Immunochip | Coronary artery disease ∥ id: ebi-a-GCST005194 | Simple
mode | 250 | −0.0121726 | 0.01609973 | 0.45031979; All
SNPs | SLE_Immunochip | Coronary artery disease ∥ id: ebi-a-GCST005194 | Weighted
mode | 250 | 0.00527088 | 0.0099465 | 0.59663716; All
SNPs | SLE_Immunochip | Coronary artery disease ∥ id: ebi-a-GCST005194 | Weighted
mode (NOME) | 250 | 0.00527088 | 0.00893171 | 0.5556377; All
SNPs | SLE_Immunochip | Coronary artery disease ∥ id: ebi-a-GCST005194 | Simple mode
(NOME) | 250 | −0.0121726 | 0.01608942 | 0.45002974; All
SNPs | SLE_Immunochip | Coronary artery disease ∥ id: ebi-a-GCST005194 | Robust
adjusted profile score (RAPS) | 250 | 0.01161658 | 0.00483635 | 0.01630883; All
SNPs | SLE_Immunochip | Coronary artery disease ∥ id: ebi-a-GCST005194 | MR-PRESSO
(raw) | 250 | 0.01266393 | 0.00461884 | 0.00655367; All
SNPs | SLE_Immunochip | Coronary artery disease ∥ id: ebi-a-GCST005194 | MR-PRESSO
(outlier-corrected) | 245 | 0.01185503 | 0.00432168 | 0.00653675; Positive-causal Tier 1
and Tier 2 | SLE_Immunochip | Coronary artery disease ∥ id: ebi-a-
GCST005194 | Maximum likelihood | 158 | 0.01452796 | 0.00436028 | 0.0008626;
Positive-causal Tier 1 and Tier 2 | SLE_Immunochip | Coronary artery disease ∥ id: ebi-a-
GCST005194 | MR Egger | 158 | 0.01830587 | 0.01192641 | 0.12683355; Positive-causal
Tier 1 and Tier 2 | SLE_Immunochip | Coronary artery disease ∥ id: ebi-a-
GCST005194 | MR Egger (bootstrap) | 158 | 0.01154169 | 0.00900306 | 0.104; Positive-
causal Tier 1 and Tier 2 | SLE_Immunochip | Coronary artery disease ∥ id: ebi-a-
GCST005194 | Simple median | 158 | 0.01269369 | 0.00722147 | 0.07878668; Positive-
causal Tier 1 and Tier 2 | SLE_Immunochip | Coronary artery disease ∥ id: ebi-a-
GCST005194 | Weighted median | 158 | 0.01695522 | 0.00730158 | 0.02022596; Positive-
causal Tier 1 and Tier 2 | SLE_Immunochip | Coronary artery disease ∥ id: ebi-a-
GCST005194 | Penalised weighted median | 158 | 0.01708549 | 0.00727902 | 0.01891393;
Positive-causal Tier 1 and Tier 2 | SLE_Immunochip | Coronary artery disease ∥ id: ebi-a-
GCST005194 | Inverse variance weighted | 158 | 0.01344975 | 0.00562456 | 0.01679102;
Positive-causal Tier 1 and Tier 2 | SLE_Immunochip | Coronary artery disease ∥ id: ebi-a-
GCST005194 | Inverse variance weighted (multiplicative random
effects) | 158 | 0.01344975 | 0.00562456 | 0.01679102; Positive-causal Tier 1 and Tier
2 | SLE_Immunochip | Coronary artery disease ∥ id: ebi-a-GCST005194 | Inverse variance
weighted (fixed effects) | 158 | 0.01344975 | 0.00397622 | 0.00071818; Positive-causal Tier
1 and Tier 2 | SLE_Immunochip | Coronary artery disease ∥ id: ebi-a-GCST005194 | Simple
mode | 158 | 0.03019639 | 0.01579163 | 0.05767463; Positive-causal Tier 1 and Tier
2 | SLE_Immunochip | Coronary artery disease ∥ id: ebi-a-GCST005194 | Weighted
mode | 158 | 0.02427805 | 0.00950485 | 0.01159237; Positive-causal Tier 1 and Tier
2 | SLE_Immunochip | Coronary artery disease ∥ id: ebi-a-GCST005194 | Weighted mode
(NOME) | 158 | 0.02427805 | 0.00933426 | 0.01018462; Positive-causal Tier 1 and Tier
2 | SLE_Immunochip | Coronary artery disease ∥ id: ebi-a-GCST005194 | Simple mode
(NOME) | 158 | 0.03019639 | 0.01610148 | 0.06259732; Positive-causal Tier 1 and Tier
2 | SLE_Immunochip | Coronary artery disease ∥ id: ebi-a-GCST005194 | Robust adjusted
profile score (RAPS) | 158 | 0.01301722 | 0.00610672 | 0.03303777; Positive-causal Tier 1
and Tier 2 | SLE_Immunochip | Coronary artery disease ∥ id: ebi-a-GCST005194 | MR-
PRESSO (raw) | 158 | 0.01344975 | 0.00562456 | 0.01797469; Positive-causal Tier 1 and
Tier 2 | SLE_Immunochip | Coronary artery disease ∥ id: ebi-a-GCST005194 | MR-
PRESSO (outlier-corrected) | 155 | 0.01211297 | 0.0052847 | 0.02325556; Positive-causal
Tier 1 | SLE_Immunochip | Coronary artery disease ∥ id: ebi-a-GCST005194 | Maximum
likelihood | 129 | 0.0160215 | 0.00480981 | 0.00086533; Positive-causal Tier
1 | SLE_Immunochip | Coronary artery disease ∥ id: ebi-a-GCST005194 | MR
Egger | 129 | 0.02075489 | 0.01286819 | 0.10925197; Positive-causal Tier
1 | SLE_Immunochip | Coronary artery disease ∥ id: ebi-a-GCST005194 | MR Egger
(bootstrap) | 129 | 0.0225367 | 0.0094175 | 0.009; Positive-causal Tier
1 | SLE_Immunochip | Coronary artery disease ∥ id: ebi-a-GCST005194 | Simple
median | 129 | 0.01087301 | 0.00770211 | 0.15804037; Positive-causal Tier
1 | SLE_Immunochip | Coronary artery disease ∥ id: ebi-a-GCST005194 | Weighted
median | 129 | 0.01754756 | 0.00765771 | 0.02193514; Positive-causal Tier
1 | SLE_Immunochip | Coronary artery disease ∥ id: ebi-a-GCST005194 | Penalised
weighted median | 129 | 0.01755902 | 0.00787559 | 0.02577744; Positive-causal Tier
1 | SLE_Immunochip | Coronary artery disease ∥ id: ebi-a-GCST005194 | Inverse variance
weighted | 129 | 0.01453428 | 0 .00635003 | 0.02208786; Positive-causal Tier
1 | SLE_Immunochip | Coronary artery disease ∥ id: ebi-a-GCST005194 | Inverse variance
weighted (multiplicative random effects) | 129 | 0.01453428 | 0.00635003 | 0.02208786;
Positive-causal Tier 1 | SLE_Immunochip | Coronary artery disease ∥ id: ebi-a-
GCST005194 | Inverse variance weighted (fixed
effects) | 129 | 0.01453428 | 0.00433616 | 0.00080266; Positive-causal Tier
1 | SLE_Immunochip | Coronary artery disease ∥ id: ebi-a-GCST005194 | Simple
mode | 129 | 0.0275948 | 0.01531548 | 0.07393698; Positive-causal Tier
1 | SLE_Immunochip | Coronary artery disease ∥ id: ebi-a-GCST005194 | Weighted
mode | 129 | 0.02340909 | 0.01003113 | 0.02117278; Positive-causal Tier
1 | SLE_Immunochip | Coronary artery disease ∥ id: ebi-a-GCST005194 | Weighted mode
(NOME) | 129 | 0.02340909 | 0.00892355 | 0.00976629; Positive-causal Tier
1 | SLE_Immunochip | Coronary artery disease ∥ id: ebi-a-GCST005194 | Simple mode
(NOME) | 129 | 0.0275948 | 0.0161255 | 0.08945678; Positive-causal Tier
1 | SLE_Immunochip | Coronary artery disease ∥ id: ebi-a-GCST005194 | Robust adjusted
profile score (RAPS) | 129 | 0.0129632 | 0.00693128 | 0.06144938; Positive-causal Tier
1 | SLE_Immunochip | Coronary artery disease ∥ id: ebi-a-GCST005194 | MR-PRESSO
(raw) | 129 | 0.01453428 | 0.00635003 | 0.02372629; Positive-causal Tier
1 | SLE_Immunochip | Coronary artery disease ∥ id: ebi-a-GCST005194 | MR-PRESSO
(outlier-corrected) | 124 | 0.01115835 | 0.00567814 | 0.0516518; Positive-causal MR-IVW
p-value < 0.00075 | SLE_Immunochip | Coronary artery disease ∥ id: ebi-a-
GCST005194 | Maximum likelihood | 115 | 0.02034331 | 0.00518792 | 8.81E−05; Positive-
causal MR-IVW p-value < 0.00075 | SLE_Immunochip | Coronary artery disease ∥ id: ebi-a-
GCST005194 | MR Egger | 115 | 0.03056855 | 0.01344607 | 0.02489041; Positive-causal
MR-IVW p-value < 0.00075 | SLE_Immunochip | Coronary artery disease ∥ id: ebi-a-
GCST005194 | MR Egger (bootstrap) | 115 | 0.02979344 | 0.01095336 | 0.002; Positive-
causal MR-IVW p-value < 0.00075 | SLE_Immunochip | Coronary artery disease ∥ id: ebi-a-
GCST005194 | Simple median | 115 | 0.01600021 | 0.00832895 | 0.05472723; Positive-
causal MR-IVW p-value < 0.00075 | SLE_Immunochip | Coronary artery disease ∥ id: ebi-a-
GCST005194 | Weighted median | 115 | 0.02707248 | 0.00837499 | 0.00122695; Positive-
causal MR-IVW p-value < 0.00075 | SLE_Immunochip | Coronary artery disease ∥ id: ebi-a-
GCST005194 | Penalised weighted median | 115 | 0.02707911 | 0.00854154 | 0.00152291;
Positive-causal MR-IVW p-value < 0.00075 | SLE_Immunochip | Coronary artery disease ∥
id: ebi-a-GCST005194 | Inverse variance weighted | 115 | 0.01835041 | 0.0064594 | 4.50E−
03; Positive-causal MR-IVW p-value < 0.00075 | SLE_Immunochip | Coronary artery
disease | id: ebi-a-GCST005194 | Inverse variance weighted (multiplicative random
effects) | 115 | 0.01835041 | 0.0064594 | 4.50E−03; Positive-causal MR-IVW p-
value < 0.00075 | SLE_Immunochip | Coronary artery disease ∥ id: ebi-a-
GCST005194 | Inverse variance weighted (fixed
effects) | 115 | 0.01835041 | 0.00467005 | 8.52E−05; Positive-causal MR-IVW p-
value < 0.00075 | SLE_Immunochip | Coronary artery disease ∥ id: ebi-a-
GCST005194 | Simple mode | 115 | 0.02531232 | 0.01615301 | 0.11987842; Positive-
causal MR-IVW p-value < 0.00075 | SLE_Immunochip | Coronary artery disease ∥ id: ebi-a-
GCST005194 | Weighted mode | 115 | 0.0273921 | 0.01081231 | 0.01265407; Positive-
causal MR-IVW p-value < 0.00075 | SLE_Immunochip | Coronary artery disease ∥ id: ebi-a-
GCST005194 | Weighted mode (NOME) | 115 | 0.02947187 | 0.01024785 | 0.00480916;
Positive-causal MR-IVW p-value < 0.00075 | SLE_Immunochip | Coronary artery disease ∥
id: ebi-a-GCST005194 | Simple mode
(NOME) | 115 | 0.02531232 | 0.01804452 | 0.1634028; Positive-causal MR-IVW p-
value < 0.00075 | SLE_Immunochip | Coronary artery disease ∥ id: ebi-a-
GCST005194 | Robust adjusted profile score
(RAPS) | 115 | 0.02113771 | 0.00726291 | 3.61E−03; Positive-causal MR-IVW p-
value < 0.00075 | SLE_Immunochip | Coronary artery disease ∥ id: ebi-a-
GCST005194 | MR-PRESSO (raw) | 115 | 0.01835041 | 0.0064594 | 0.0053303; Positive-
causal MR-IVW p-value < 0.00075 | SLE_Immunochip | Coronary artery disease ∥ id: ebi-a-
GCST005194 | MR-PRESSO (outlier-
corrected) | 112 0.01989322 | 0.00597268 | 0.00117678; Negative-causal Tier 1 and Tier
2 | SLE_Immunochip | Coronary artery disease ∥ id: ebi-a-GCST005194 | Maximum
likelihood | 54 | −0.0136133 | 0.00724621 | 0.06028873; Negative-causal Tier 1 and Tier
2 | SLE_Immunochip | Coronary artery disease ∥ id: ebi-a-GCST005194 | MR Egger | 54 | −0.0184199 |
0.02402969 | 0.44681742; Negative-causal Tier 1 and Tier
2 | SLE_Immunochip | Coronary artery disease ∥ id: ebi-a-GCST005194 | MR Egger
(bootstrap) | 54 | 0.00653564 | 0.01415626 | 0.34; Negative-causal Tier 1 and Tier
2 | SLE_Immunochip | Coronary artery disease ∥ id: ebi-a-GCST005194 | Simple
median | 54 | −0.016065 | 0.01291408 | 0.21350243; Negative-causal Tier 1 and Tier
2 | SLE_Immunochip | Coronary artery disease ∥ id: ebi-a-GCST005194 | Weighted
median | 54 | −0.0093167 | 0.0108865 | 0.39210566; Negative-causal Tier 1 and Tier
2 | SLE_Immunochip | Coronary artery disease ∥ id: ebi-a-GCST005194 | Penalised
weighted median | 54 | −0.0072026 | 0.01146249 | 0.52976661; Negative-causal Tier 1 and
Tier 2 | SLE_Immunochip | Coronary artery disease ∥ id: ebi-a-GCST005194 | Inverse
variance weighted | 54 | −0.0126818 | 0.01096282 | 0.24735398; Negative-causal Tier 1 and
Tier 2 | SLE_Immunochip | Coronary artery disease ∥ id: ebi-a-GCST005194 | Inverse
variance weighted (multiplicative random effects) | 54 | −0.0126818 |
0.01096282 | 0.24735398; Negative-causal Tier 1 and Tier
2 | SLE_Immunochip | Coronary artery disease ∥ id: ebi-a-GCST005194 | Inverse variance
weighted (fixed effects) | 54 | −0.0126818 | 0.00677109 | 0.06107771; Negative-causal Tier
1 and Tier 2 | SLE_Immunochip | Coronary artery disease ∥ id: ebi-a-GCST005194 | Simple
mode | 54 | −0.0127362 | 0.02295917 | 0.58140988; Negative-causal Tier 1 and Tier
2 | SLE_Immunochip | Coronary artery disease ∥ id: ebi-a-GCST005194 | Weighted
mode | 54 | −0.0004983 | 0.01256046 | 0.96850404; Negative-causal Tier 1 and Tier
2 | SLE_Immunochip | Coronary artery disease ∥ id: ebi-a-GCST005194 | Weighted mode
(NOME) | 54 | −0.0004983 | 0.01321525 | 0.97006381; Negative-causal Tier 1 and Tier
2 | SLE_Immunochip | Coronary artery disease ∥ id: ebi-a-GCST005194 | Simple mode
(NOME) | 54 | −0.0127362 | 0.02489304 | 0.61102908; Negative-causal Tier 1 and Tier
2 | SLE_Immunochip | Coronary artery disease ∥ id: ebi-a-GCST005194 | Robust adjusted
profile score (RAPS) | 54 | −0.0189141 | 0.01187237 | 0.11113319; Negative-causal Tier 1
and Tier 2 | SLE_Immunochip | Coronary artery disease ∥ id: ebi-a-GCST005194 | MR-
PRESSO (raw) | 54 |−0.0126818 | 0.01096282 | 0.25254142; Negative-causal Tier 1 and
Tier 2 | SLE_Immunochip | Coronary artery disease ∥ id: ebi-a-GCST005194 | MR-
PRESSO (outlier-corrected) | 52 | −0.0128084 | 0.01019389 | 0.21466899; Negative-causal
Tier 1 | SLE_Immunochip | Coronary artery disease ∥ id: ebi-a-GCST005194 | Maximum
likelihood | 36 | −0.048655 | 0.01066058 | 5.0185E−06; Negative-causal Tier
1 | SLE_Immunochip | Coronary artery disease ∥ id: ebi-a-GCST005194 | MR Egger | 36 | −0.0869952 |
0.03702208 | 0.02472765; Negative-causal Tier
1 | SLE_Immunochip | Coronary artery disease ∥ id: ebi-a-GCST005194 | MR Egger
(bootstrap) | 36 | −0.0591592 | 0.02791304 | 0.019; Negative-causal Tier
1 | SLE_Immunochip | Coronary artery disease ∥ id: ebi-a-GCST005194 | Simple
median | 36 | −0.0328077 | 0.01604377 | 0.04086618; Negative-causal Tier
1 | SLE_Immunochip | Coronary artery disease ∥ id: ebi-a-GCST005194 | Weighted
median | 36 | −0.0333915 | 0.01633625 | 0.04095241; Negative-causal Tier
1 | SLE_Immunochip | Coronary artery disease ∥ id: ebi-a-GCST005194 | Penalised
weighted median | 36 | −0.0343239 | 0.01659368 | 0.03859371; Negative-causal Tier
1 | SLE_Immunochip | Coronary artery disease ∥ id: ebi-a-GCST005194 | Inverse variance
weighted | 36 | −0.0441376 | 0.01638592 | 0.00706789; Negative-causal Tier
1 | SLE_Immunochip | Coronary artery disease ∥ id: ebi-a-GCST005194 | Inverse variance
weighted (multiplicative random effects) | 36 | −0.0441376 | 0.01638592 | 0.00706789;
Negative-causal Tier 1 | SLE_Immunochip | Coronary artery disease ∥ id: ebi-a-
GCST005194 | Inverse variance weighted (fixed effects) | 36 | −0.0441376 |
0.0097068 | 5.4395E−06; Negative-causal Tier
1 | SLE_Immunochip | Coronary artery disease ∥ id: ebi-a-GCST005194 | Simple
mode | 36 | −0.0316247 | 0.02903104 | 0.28344803; Negative-causal Tier
1 | SLE_Immunochip | Coronary artery disease ∥ id: ebi-a-GCST005194 | Weighted
mode | 36 | −0.0417143 | 0.01782945 | 0.0251354; Negative-causal Tier
1 | SLE_Immunochip | Coronary artery disease ∥ id: ebi-a-GCST005194 | Weighted mode
(NOME) | 36 | −0.0484406 | 0.01923614 | 0.01652102; Negative-causal Tier
1 | SLE_Immunochip | Coronary artery disease ∥ id: ebi-a-GCST005194 | Simple mode
(NOME) | 36 | −0.0316247 | 0.03063472 | 0.30900581; Negative-causal Tier
1 | SLE_Immunochip | Coronary artery disease ∥ id: ebi-a-GCST005194 | Robust adjusted
profile score (RAPS) | 36 | −0.0524318 | 0.01633032 | 0.00132411; Negative-causal Tier
1 | SLE_Immunochip | Coronary artery disease ∥ id: ebi-a-GCST005194 | MR-PRESSO
(raw) | 36 | −0.0441376 | 0.01638592 | 0.01077774; Negative-causal Tier
1 | SLE_Immunochip | Coronary artery disease ∥ id: ebi-a-GCST005194 | MR-PRESSO
(outlier-corrected) | 32 | −0.0552726 | 0.01268263 | 0.0001338; Negative-causal MR-IVW
p-value < 0.00075 | SLE_Immunochip | Coronary artery disease ∥ id: ebi-a-
GCST005194 | Maximum likelihood | 42 | −0.0433335 | 0.00936341 | 3.6928E−06;
Negative-causal MR-IVW p-value < 0.00075 | SLE_Immunochip | Coronary artery disease ∥
id: ebi-a-GCST005194 | MR Egger | 42 | −0.0626122 | 0.03340908 | 0.068233; Negative-
causal MR-IVW p-value < 0.00075 | SLE_Immunochip | Coronary artery disease ∥ id: ebi-a-
GCST005194 | MR Egger (bootstrap) | 42 | −0.0446436 | 0.02587872 | 0.042; Negative-
causal MR-IVW p-value < 0.00075 | SLE_Immunochip | Coronary artery disease ∥ id: ebi-a-
GCST005194 | Simple median | 42 | −0.0284403 | 0.01428402 | 0.04647485; Negative-
causal MR-IVW p-value < 0.00075 | SLE_Immunochip | Coronary artery disease ∥ id: ebi-a-
GCST005194 | Weighted median | 42 | −0.0282995 | 0.01399869 | 0.04321954; Negative-
causal MR-IVW p-value < 0.00075 | SLE_Immunochip | Coronary artery disease ∥ id: ebi-a-
GCST005194 | Penalised weighted median | 42 | −0.0283187 | 0.01439378 | 0.04913413;
Negative-causal MR-IVW p-value < 0.00075 | SLE_Immunochip | Coronary artery disease ∥
id: ebi-a-GCST005194 | Inverse variance weighted | 42 | −0.0386569 |
0.01419302 | 0.00645636; Negative-causal MR-IVW p-
value < 0.00075 | SLE_Immunochip | Coronary artery disease ∥ id: ebi-a-
GCST005194 | Inverse variance weighted (multiplicative random effects) | 42 | −0.0386569 |
0.01419302 | 0.00645636; Negative-causal MR-IVW p-
value < 0.00075 | SLE_Immunochip | Coronary artery disease ∥ id: ebi-a-
GCST005194 | Inverse variance weighted (fixed effects) | 42 | −0.0386569 |
0.00865344 | 7.9239E−06; Negative-causal MR-IVW p-
value < 0.00075 | SLE_Immunochip | Coronary artery disease ∥ id: ebi-a-
GCST005194 | Simple mode | 42 | −0.0186047 | 0.0257424 | 0.47395195; Negative-causal
MR-IVW p-value < 0.00075 | SLE_Immunochip | Coronary artery disease ∥ id: ebi-a-
GCST005194 | Weighted mode | 42 | −0.0249463 | 0.01686732 | 0.14678961; Negative-
causal MR-IVW p-value < 0.00075 | SLE_Immunochip | Coronary artery disease ∥ id: ebi-a-
GCST005194 | Weighted mode (NOME) | 42 | −0.0265318 | 0.01835982 | 0.15603012;
Negative-causal MR-IVW p-value < 0.00075 | SLE_Immunochip | Coronary artery disease ∥
id: ebi-a-GCST005194 | Simple mode (NOME) | 42 | −0.0186047 |
0.02655042 | 0.48742982; Negative-causal MR-IVW p-
value < 0.00075 | SLE_Immunochip | Coronary artery disease ∥ id: ebi-a-
GCST005194 | Robust adjusted profile score (RAPS) | 42 | −0.0446155 |
0.01444644 | 2.01E−03; Negative-causal MR-IVW p-
value < 0.00075 | SLE_Immunochip | Coronary artery disease ∥ id: ebi-a-
GCST005194 | MR-PRESSO (raw) | 42 | −0.0386569 | 0.01419302 | 0.00944305; Negative-
causal MR-IVW p-value < 0.00075 | SLE_Immunochip | Coronary artery disease ∥ id: ebi-a-
GCST005194 | MR-PRESSO (outlier-corrected) | 38 | −0.0470622 |
0.01146131 | 0.00021305; Insignificant | SLE_Immunochip | Coronary artery
disease ∥ id: ebi-a-GCST005194 | Maximum
likelihood | 136 | 0.01362361 | 0.0047226 | 0.00391704;
Insignificant | SLE_Immunochip | Coronary artery disease ∥ id: ebi-a-GCST005194 | MR
Egger | 136 | −0.0016329 | 0.01441888 | 0.91000618;
Insignificant | SLE_Immunochip | Coronary artery disease ∥ id: ebi-a-GCST005194 | MR
Egger (bootstrap) | 136 | 0.00691259 | 0.01082987 | 0.261;
Insignificant | SLE_Immunochip | Coronary artery disease ∥ id: ebi-a-
GCST005194 | Simple median | 136 | 0.00456323 | 0.0076355 | 0.55008456;
Insignificant | SLE_Immunochip | Coronary artery disease ∥ id: ebi-a-
GCST005194 | Weighted median | 136 | 0.00210469 | 0.00806598 | 0.79414355;
Insignificant | SLE_Immunochip | Coronary artery disease ∥ id: ebi-a-
GCST005194 | Penalised weighted median | 136 | 0.00188309 | 0.00795639 | 0.81290797;
Insignificant | SLE_Immunochip | Coronary artery disease ∥ id: ebi-a-
GCST005194 | Inverse variance weighted | 136 | 0.012596 | 0.00679481 | 0.06377255;
Insignificant | SLE_Immunochip | Coronary artery disease ∥ id: ebi-a-
GCST005194 | Inverse variance weighted (multiplicative random
effects) | 136 | 0.012596 | 0.00679481 | 0.06377255;
Insignificant | SLE_Immunochip | Coronary artery disease ∥ id: ebi-a-
GCST005194 | Inverse variance weighted (fixed
effects) | 136 | 0.012596 | 0.00442807 | 0.00444702;
Insignificant | SLE_Immunochip | Coronary artery disease ∥ id: ebi-a-
GCST005194 | Simple mode | 136 | −0.0016151 | 0.01853292 | 0.93068217;
Insignificant | SLE_Immunochip | Coronary artery disease ∥ id: ebi-a-
GCST005194 | Weighted mode | 136 | 0.00480095 | 0.01030854 | 0.64216222;
Insignificant | SLE_Immunochip | Coronary artery disease ∥ id: ebi-a-
GCST005194 | Weighted mode (NOME) | 136 | 0.00480095 0.0108731 | 0.65952553;
Insignificant | SLE_Immunochip | Coronary artery disease ∥ id: ebi-a-
GCST005194 | Simple mode (NOME) | 136 | −0.0016151 | 0.01810749 | 0.92905787;
Insignificant | SLE_Immunochip | Coronary artery disease ∥ id: ebi-a-
GCST005194 | Robust adjusted profile score
(RAPS) | 136 | 0.0101828 | 0.00700973 | 0.14631658;
Insignificant | SLE_Immunochip | Coronary artery disease ∥ id: ebi-a-GCST005194 | MR-
PRESSO (raw) | 136 | 0.012596 | 0.00679481 | 0.06595511;
Insignificant | SLE_Immunochip | Coronary artery disease ∥ id: ebi-a-GCST005194 | MR-
PRESSO (outlier-corrected) | 130 | 0.00989766 | 0.00608074 | 0.1060275;

TABLE 4
Pathway analysis of SLE IVs determined to be causal of CAD by traditional MR.
Gene set enrichments for each cluster were determined using IPA and the EnrichR library database.
P-values from Fisher's exact test measures the significance of overlap between
genes in each cluster and genes within an annotation.

| | | P- | | P- | |
|---|---|---|---|---|---|
| Cluster | IPA | value | EnrichR | value | Dataset |
| 1 | Semaphorin | 0.000304 | B lymphocyte cell surface molecules | 1.64E−07 | BioPlanet |
| | neuronal | | | | |
| | repulsive | | | | |
| | signaling | | | | |
| | pathway | | | | |
| | Leukocyte | 0.000623 | systemic lupus erythematosus | 5.74E−05 | ClinVar |
| | extravasation | | | | 2019 |
| | signaling | | | | |
| | IL-10 | 0.00182 | myocardial infarction 1 | 0.01357 | Clin Var |
| | signaling | | | | 2019 |
| | Crosstalk | | | | |
| | between | 0.00288 | Fc receptor complex | 2.79E−08 | Jensen |
| | dendritic cells | | | | COMPARTMENTS |
| | and NK cells | | | | |
| | TH2 pathway | 0.0064 | IgG immunoglobulin complex | 6.59E−08 | Jensen |
| | | | | | COMPARTMENTS |
| | | | interleukin-12 complex | 5.30E−07 | Jensen |
| | | | | | COMPARTMENTS |
| | | | High endothelial venule | 4.85E−06 | Jensen |
| | | | | | TISSUES |
| | | | | | GO |
| | | | regulation of immune response | 9.84E−07 | Biological |
| | | | (GO: 0050776) | | Process |
| | | | | | 2021 |
| 2 | Role of | | | | |
| | hypercytokine | 0.00234 | negative regulation of B cell activation | 1.30E−06 | GO |
| | mia/hyperchemokinemia | | (GO: 0050869) | | Biological |
| | on the | | | | Process |
| | pathogenesis | | | | 2021 |
| | of influenza | | | | |
| | Systemic | 0.00275 | Interferon regulatory factor 5 complex | 1.01E−13 | Jensen |
| | lupus | | | | COMPARTMENTS |
| | erythematosus | | | | |
| | in B cell | | | | |
| | signaling | | | | |
| | pathway | | | | |
| | IL-13 | 0.00421 | interleukin-35 complex | 6.65E−09 | Jensen |
| | signaling | | | | COMPARTMENTS |
| | pathway | | | | |
| | Role of tissue | 0.00421 | Interferon regulatory factor complex | 8.34E−08 | Jensen |
| | factor in | | | | COMPARTMENTS |
| | cancer | | | | |
| | TH1 pathway | 0.00464 | interleukin-12 complex | 3.61E−07 | Jensen |
| | | | | | COMPARTMENTS |
| | | | interleukin-23 complex | 4.02E−06 | Jensen |
| | | | | | COMPARTMENTS |
| | | | Toll-like receptor signaling pathway | 1.46E−04 | KEGG |
| | | | | | 2021 |
| | | | | | Human |
| | | | Lipid and atherosclerosis | 1.22E−03 | KEGG |
| | | | | | 2021 |
| | | | | | Human |
| | | | Vitamin B-complex deficiencies | 1.47E−08 | PheWeb |
| | | | | | 2019 |
| | | | Systemic lupus erythematosus | 2.06E−27 | GWAS |
| | | | | | Catalog |
| | | | | | 2019 |
| 3 | Acute myeloid | 0.00441 | response to anticoagulant | 1.32E−08 | DisGeNET |
| | leukemia | | | | |
| | signaling | | | | |
| | ATM | 0.00519 | Systemic lupus erythematosus | 3.11E−07 | GWAS |
| | signaling | | | | Catalog |
| | | | | | 2019 |
| | Kinetochore | 0.00648 | aicardi goutieres syndrome | 0.006484 | ClinVar |
| | metaphase | | | | 2019 |
| | signaling | | | | |
| | pathway | | | | |
| | Ferroptosis | 0.009 | Hypothyroidism (Hashimoto's disease) | 0.000996 | PheWeb |
| | signaling | | | | 2019 |
| | pathway | | | | |
| | HOTAIR | 0.0135 | Arterial embolism and thrombosis | 0.000369 | PheWeb |
| | regulatory | | | | 2019 |
| | pathway | | | | |
| | | | beta-catenin-TCF complex assembly | 6.97E−06 | GO |
| | | | (GO: 1904837) | | Biological |
| | | | | | Process |
| | | | | | 2021 |
| | | | histone acetyltransferase activity | 8.56E−06 | GO |
| | | | (GO: 0004402) | | Molecular |
| | | | | | Function |
| | | | | | 2021 |
| | | | ATM Signaling Network in Development | 2.67E−05 | WikiPathway |
| | | | and Disease WP3878 | | 2021 |
| | | | | | Human |
| 4 | Arsenate | 0.00188 | Pyrimidine metabolism | 0.000274 | KEGG |
| | detoxification | | | | 2021 |
| | I | | | | Human |
| | Guanosine | 0.00489 | Metabolism of nucleotides Homo sapiens | 0.00066 | |
| | nucleotides | | R-HSA-15869 | | |
| | degradation III | | | | |
| | Urate | 0.00527 | Abacavir metabolism Homo sapiens R- | Reactome 2016 | |
| | biosunthesis/ | | HSA-2161541 | | |
| | inosine 5′- | | | | |
| | phosphate | | | | |
| | degradation | | | | |
| | Adenosine | 0.00602 | | 3.41E−05 | Jensen |
| | nucleotides | | Mitochondrial proton-transporting ATP | | COMPARTMENTS |
| | degradation II | | synthase complex | | |
| | Purine | 0.00715 | Hypertension | 3.88E−06 | PhenGenI |
| | nucleotides | | | | Association |
| | degradation II | | | | 2021 |
| | | | Arterial Pressure | 0.00024 | PhenGenI |
| | | | | | Association |
| | | | | | 2021 |
| | | | Plaque, Atherosclerotic | 0.00096 | PhenGenI |
| | | | | | Association |
| | | | | | 2021 |
| | | | Mean blood pressure | 9.44E−05 | DisGeNET |
| 5 | Production of | 6.26E−08 | interleukin-23 & 27-mediated signaling | 6.28E−05 | GO |
| | No and ROS | | pathway (GO: 0038155) | | Biological |
| | in | | | | Process |
| | macrophages | | | | 2021 |
| | Oncostatin M | 1.56E−07 | growth hormone receptor binding | 6.28E−05 | GO |
| | signaling | | (GO: 0005131) | | Molecular |
| | | | | | Function |
| | | | | | 2021 |
| | STAT3 | 3.93E−07 | caveola (GO: 0005901) | 7.13E−05 | GO Cellular |
| | pathway | | | | Compartment |
| | | | | | 2021 |
| | CNTF | 4.93E−07 | Blood platelet | 0.000151 | Jensen |
| | signaling | | | | TISSUES |
| | SPINK1 | 1.97E−06 | interleukin-23 complex | 1.83E−06 | Jensen |
| | general cancer | | | | COMPARTMENTS |
| | pathway | | | | |
| | | | interleukin-12 complex | 3.55E−06 | Jensen |
| | | | | | COMPARTMENTS |
| | | | Carotid Stenosis | 0.02271 | dbGaP |
| | | | Systemic lupus erythematosus | 1.55E−14 | GWAS |
| | | | | | Catalog |
| | | | | | 2019 |
| | | | Autoantibodies | 1.88E−07 | PhenGenI |
| | | | | | Association |
| | | | | | 2021 |
| | | | Lupus Erythematosus, Systemic | 1.46−11 | PhenGenI |
| | | | | | Association |
| | | | | | 2021 |
| 6 | No enrichment | | Interleukin-2 signaling pathway (all 3 | 7.57E−05 | |
| | | | genes) | | BioPlanet |
| | | | | | 2019 |
| | | | regulation of cytokine production | 4.14E−07 | GO |
| | | | (GO:0001817) | | Biological |
| | | | | | Process |
| | | | | | 2021 |
| | | | T cell receptor signaling pathway | 4.84E−07 | GO |
| | | | (GO: 0050852) | | Biological |
| | | | | | Process |
| | | | | | 2021 |
| | | | antigen receptor-mediated signaling | 7.79E−07 | GO |
| | | | pathway (GO: 0050851) | | Biological |
| | | | | | Process |
| | | | | | 2021 |
| | | | gamma-delta T cell receptor complex | 2.75E−07 | Jensen |
| | | | | | COMPARTMENTS |
| 7 | G alpha (i) | 0.00149 | Dopamine receptors | 2.50E−03 | BioPlanet |
| | signaling | | | | 2019 |
| | G protein | 0.0183 | G alpha (i) signaling events | 4.21E−03 | BioPlanet |
| | signaling | | | | 2019 |
| | mediated by | | | | |
| | tybby | | | | |
| | Apelin muscle | 0.0199 | NPHP complex | 7.67E−04 | Jensen |
| | signaling | | | | COMPARTMENTS |
| | pathway | | | | |
| | Breat cancer | 0.0244 | Aortic valve stenosis | 4.99E−03 | Jensen |
| | regulation by | | | | Diseases |
| | stathmin 1 | | | | |
| | CREB | 0.0253 | Systemic lupus erythematosus | 0.000658 | GWAS |
| | signlaing in | | | | Catalog |
| | neurons | | | | 2019 |
| 8 | Oxidized GTP | 0.0017 | positive regulation of basement membrane | 1.75E−03 | GO |
| | and dGTP | | assembly involved in embryonic body | | Biological |
| | detoxification | | morphogenesis (GO: 1904261) | | Process |
| | | | | | 2021 |
| | Trehalose | 0.00205 | telomerase RNA binding (GO: 0070034) | 7.68E−03 | GO |
| | degradation II | | | | Molecular |
| | | | | | Function |
| | | | | | 2021 |
| | Coronavius | 0.0131 | COPI vesicle coat (GO: 0030126) | 0.004542 | GO Cellular |
| | replication | | | | Compartment |
| | pathway | | | | 2021 |
| | CSDE1 | 0.0163 | RISC complex (GO: 0016442) | 0.004542 | GO Cellular |
| | signaling | | | | Compartment |
| | pathway | | | | 2021 |
| | Caveolar- | 0.0218 | Autoimmune Diseases | 0.000471 | PhenGenI |
| | mediated | | | | Association |
| | endocytosis | | | | 2021 |
| | signaling | | | | |
| 9 | Choline | 5.53E−06 | glycerophospholipid biosynthetic process | 1.34E−05 | GO |
| | biosynthesis | | (GO: 0046474) | | Biological |
| | III | | | | Process 2021 |
| | Phosphatidylet | 0.000251 | platelet activating factor metabolic process | 0.002398 | GO |
| | hanolamine | | (GO: 0046469) | | Biological |
| | biosynthesis | | | | Process |
| | ((( | | | | 2021 |
| | Selenocysteine | 0.00151 | Type 2 diabetes with neurological | 2.24E−05 | PheWeb |
| | biosynthesis | | manifestations | | 2019 |
| | II | | | | |
| | Phosphatidylcholine | 0.00176 | Anticoagulants | 0.01135 | PhenGenI |
| | biosynthesis I | | | | Association |
| | | | | | 2021 |
| | Phospholipases | 0.0167 | Sphingolipid levels | 0.00509 | GWAS |
| | | | | | Catalog |
| | | | | | 2019 |
| 10 | Dolichyl- | 0.00293 | Bile acid and bile salt biosynthesis via 27- | 0.002098 | BioPlanet |
| | diphosphooligosaccharide | | hydroxycholesterol | | 2019 |
| | biosynthesis | | | | |
| | Mineralcorticoid | 0.0041 | B cell chemotaxis (GO: 0035754) | 0.002098 | GO |
| | biosynthesis | | | | Biological |
| | | | | | Process |
| | | | | | 2021 |
| | Glucocorticoid | 0.00439 | Other conditions of brain, NOS | 0.005936 | PheWeb |
| | biosynthesis | | | | 2019 |
| | Bile acid | 0.00527 | Cardiac congenital anomalies | 0.008718 | PheWeb |
| | biosynthesis, | | | | 2019 |
| | neutral | | | | |
| | pathway | | | | |
| | Angrogen | 0.00615 | Ischemic Heart Disease | 0.01496 | PheWeb |
| | biosynthesis | | | | 2019 |

Table 5: Canonical pathway and disease phenotype enrichments for network analysis of positive and negative causal SNP-predicted genes determined by single-SNP MR. P-values from Fisher's exact test that measures the significance of overlap between genes in each cluster and genes within an annotation.

TABLE 5A
80 positive causal SNPs predict 236 genes.

| Cluster | IPA | P-value | EnrichR | P-value | Dataset |
|---|---|---|---|---|---|
| 1 | RAC signaling | 0.0000065 | • | 0.00029 | GO |
| | | | Phagocytosis, engulfment | | Biological |
| | | | (GO: 0006911) | | Process |
| | | | | | 2021 |
| | Complement | 0.00021 | Steroid biosynthetic process | 0.00092 | GO |
| | systems | | (GO: 0006694) | | Biological |
| | | | | | Process |
| | | | | | 2021 |
| | Sertoli Cell- | 0.00021 | Systemic lupus erythematosus | 1.1E−10 | Jensen |
| | Sertoli Cell | | | | Diseases |
| | Junction | | | | |
| | Signaling | | | | |
| | IL-8 signaling | 0.00028 | Cardiovascular system disease | 0.0006 | Jensen |
| | | | | | Diseases |
| | Osteoarthritis | 0.00032 | Systemic lupus erythematosus | 3.7E−13 | GWAS |
| | pathway | | | | catalog |
| | | | | | 2019 |
| 2 | Spliceosomal | 0.000016 | mRNA processing (GO: 0006397) | 3.9E−09 | GO |
| | Cycle | | | | Biological |
| | | | | | Process |
| | | | | | 2021 |
| | S-adenosy1-L- | 0.00302 | RNA splicing, via transesterification | 1.3E−08 | GO |
| | methionine | | reactions with bulged adenosine as | | Biological |
| | biosynthesis | | nucleophile (GO: 0000377) | | Process |
| | | | | | 2021 |
| | Embryonic stem | 0.01 | mRNA splicing, via spliceosome | 2.37E−08 | GO |
| | cell development | | (GO: 0000398) | | Biological |
| | | | | | Process |
| | | | | | 2021 |
| | Assembly of | 0.013 | Behcet syndrome | 2.30E−17 | PhenGenI |
| | RNA Polymerase | | | | Association |
| | III Complex | | | | 2021 |
| | Methionine | 0.0239 | Psoriasis | 5.80E−10 | PheWeb |
| | degradation I | | | | 2019 |
| 3 | LXR/RXR | 0.00015 | Regulation of apoptotic cell clearance | 1.399E−06 | GO |
| | Activation | | (GO: 2000425) | | Molecular |
| | | | | | Function |
| | | | | | 2021 |
| | FXR/RXR | 0.00016 | Regulation of complement activation | 0.00006 | GO |
| | Activation | | (GO: 0030449) | | Molecular |
| | | | | | Function |
| | | | | | 2021 |
| | Mineralocortocoid | 0.0023 | Diabetes mellitus, type I | 0.0013 | PhenGenI |
| | biosynthesis | | | | Association |
| | | | | | 2021 |
| | Glucocorticoid | 0.0025 | Lupus Erythematosus, Systemic | 0.0056 | PhenGenI |
| | biosynthesis | | | | Association |
| | | | | | 2021 |
| | Complement | 0.0061 | Cornary artery bypass | 0.0062 | PhenGenI |
| | systems | | | | Association |
| | | | | | 2021 |
| 4 | PTEN Signaling | 0.03 | Protein targeting (GO: 0006605) | 0.03 | GO |
| | | | | | Biological |
| | | | | | Process |
| | | | | | 2021 |
| | | | Rheumatoid arthritis | 0.00003 | Jensen |
| | | | | | Diseases |
| | | | Hypothyroidism | 0.0035 | Jensen |
| | | | | | Diseases |
| | | | Diabetes mellitus, type I | 0.0013 | PhenGenI |
| | | | | | Association |
| | | | | | 2021 |
| | | | Alopecia | 2.27E−09 | GWAS |
| | | | | | catalog |
| | | | | | 2019 |
| 5 | Estrogen- | 0.00544 | Negative regulation of fat cell | 0.0014 | GO |
| | mediated S phase | | proliferation (GO: 0070345) | | Biological |
| | entry | | | | Process |
| | | | | | 2021 |
| | Role of | 0.00606 | Granulocyte differentiation | 0.0049 | GO |
| | p14/p19ARF in | | (GO: 0070344) | | Biological |
| | tumor | | | | Process |
| | suppression | | | | 2021 |
| | Cell cycle | 0.00773 | Peroxisomal membrane | 0.013 | GO |
| | regulation by | | (GO: 0005778) | | cellular |
| | BTG family | | | | Process |
| | proteins | | | | 2021 |
| | BER pathway | 0.00919 | | | |
| | Melanoma | 0.0104 | | | |
| | signaling | | | | |
| 6 | Role of BRCA1 | 7.586E−05 | snRNA transcription by RNA | 0.000058 | GO |
| | in DNA Damage | | polymerase II (GO: 0042795) | | Biological |
| | Response | | | | Process |
| | | | | | 2021 |
| | Hereditary Breast | 0.0004169 | Cytokine-mediated signaling pathway | 0.0000073 | GO |
| | Cancer Signaling | | (GO: 0019221) | | Biological |
| | | | | | Process |
| | | | | | 2021 |
| | Ovarian Cancer | 0.0005623 | Antigen processing and presentation of | 0.000078 | GO |
| | Signaling | | peptide antigen via MHC class I | | Biological |
| | | | (GO: 0002474) | | Process |
| | | | | | 2021 |
| | Aryl | 0.0005754 | Ill defined descriptions and | 0.0006 | PheWeb |
| | Hydrocarbon | | complications of heart disease | | 2019 |
| | Receptor | | | | |
| | Signaling | | | | |
| | Assembly of | 0.0012303 | Essential hypertension | 0.018 | ClinVar |
| | RNA Polymerase | | | | 2019 |
| | II Complex | | Histone H4 acetylation (GO: 0043967) | 0.000056 | GO |
| 8 | No enrichment | | | | Biological |
| | | | | | Process |
| | | | | | 2021 |
| | | | Histone acetylation (GO: 0.0016573) | 0.00012 | GO |
| | | | | | Biological |
| | | | | | Process |
| | | | | | 2021 |
| | | | Response to anticoagulent | 0.00001 | DisGeNET |
| | | | Waist circumfrence | 0.018 | GWAS |
| | | | | | Catalog |
| | | | | | 2019 |
| | | | Chronic pulmonary heart disease | 0.000016 | PheWeb |
| | | | | | 2019 |
| 9 | Sulfite oxidation | 0.00021 | Sulfite oxidation (GO: 0019418) | 0.00129 | GO |
| | IV | | | | Biological |
| | | | | | Process |
| | | | | | 2021 |
| | Superpathway of | 0.00815 | Regulation of endocytosis | 0.01516 | GO |
| | methionine | | (GO: 0030100) | | Biological |
| | degradation | | | | Process |
| | | | | | 2021 |
| | Remodeling of | 0.0142 | Type I diabetes | 0.0002 | GWAS |
| | adherens | | | | Catalog |
| | junctions | | | | 2019 |
| | Caveolar- | 0.0156 | Pediatric autoimmune disease | 0.011 | GWAS |
| | mediated | | | | Catalog |
| | endocytosis | | | | 2019 |
| | signaling | | | | |
| | Amyotrophic | 0.0241 | Heart disease in siblings | 0.0015 | UK |
| | lateral sclerosis | | | | Biobank |
| | signaling | | | | GWAS v1 |

TABLE
96 negative causal SNPs predict 244 genes.

| Cluster | IPA | P-value | EnrichR | P-value | Dataset |
|---|---|---|---|---|---|
| 1 | Arsenate | 0.00147 | Adenosine | 0.0031 | GO Biological Process |
| | detoxification | | metabolic | | 2021 |
| | | | process | | |
| | | | (GO: 0046085) | | |
| | Guanosine | 0.00381 | Hypertension | 0.0000011 | PhenGenI Association |
| | nucleotides | | | | 2021 |
| | degradation III | | | | |
| | Urate biosynthesis | 0.0041 | Arterial pressue | 0.0001 | PhenGenI Association |
| | | | | | 2021 |
| | Adenosine | 0.00469 | Waist-to-hip | 2.50E−10 | GWAS catalog 2019 |
| | nucleotides | | ration adjusted | | |
| | degradation II | | for BMI | | |
| | Purine nucleotides | 0.00556 | Mean arterial | 2.99E−08 | GWAS catalog 2019 |
| | degradation II | | pressue (long- | | |
| | | | term average) | | |
| 2 | Cross talk between | 0.00000121 | Pyroptosis | 0.00046 | GO Biological Process |
| | dendritic cells and | | (GO: 0070269) | | 2021 |
| | NK cells | | | | |
| | NK cell signaling | 0.0000544 | Positive | 0.0027 | GO Biological Process |
| | | | regulation of | | 2021 |
| | | | leukocyte | | |
| | | | tethering or | | |
| | | | rolling | | |
| | | | (GO: 1903238) | | |
| | 14-3-3 mediated | 0.000135 | Negative | 0.0043 | GO Biological Process |
| | signaling | | regulation of B | | 2021 |
| | | | cell apoptotic | | |
| | | | process | | |
| | | | (GO: 0002903) | | |
| | Dermatan sulfate | 0.000551 | Abnormal | 0.0032 | MGI Mammalian |
| | degradation | | leukocyte | | Phenotype Level 4 |
| | | | thethering or | | 2021 |
| | | | rollin | | |
| | | | (MP:0003627) | | |
| | Adrenomedulin | 0.000742 | Thrombocytopenia | 0.004 | MGI Mammalian |
| | signaling pathway | | (MP:0003179) | | Phenotype Level 4 |
| | | | | | 2021 |
| 3 | Thrombin signaling | 0.0000267 | Regulation of | 0.0001 | GO Biological Process |
| | | | MAPK activity | | 2021 |
| | | | (GO: 0043405) | | |
| | Chenokine signaling | 0.0000338 | Response to | 0.000147 | GO Biological Process |
| | | | TNF | | 2021 |
| | | | (GO: 0034612) | | |
| | UVA-induced MAPK | 0.0000621 | Limited | 2.70E−09 | GWAS Catalog 2019 |
| | signaling | | cutaneous | | |
| | | | systemic | | |
| | | | scleroderma | | |
| | Dermatan sulfate | 0.0000811 | Parkinson's | 0.000588 | WikiPathway 2021 |
| | degradation | | disease pathway | | Human |
| | Synaptogenesis | 0.000101 | Parkinson's | 0.000002 | Jensen Diseases |
| | signaling pathway | | disease | | |
| 4 | Role of RIG-I like | 0.000429 | Positive | 4.70E−07 | GO Biological Process |
| | receptors in antiviral | | regulation oF | | 2021 |
| | innate immunity | | interferon-alpha | | |
| | | | production | | |
| | | | (GO: 0032727) | | |
| | Activation of IRF by | 0.000856 | Type I | 0.000017 | GO Biological Process |
| | cytosolic PRRs | | interferon | | 2021 |
| | | | signaling | | |
| | | | pathway | | |
| | | | (GO: 0060337) | | |
| | Dopamine receptor | 0.00126 | Mixed | 0.000054 | PheWeb 2019 |
| | signaling | | hyperlipidemia | | |
| | Role of | 0.00149 | Hypertrophy, | 0.0058 | PhenGenI Association |
| | hypercytokinemia/ | | left ventricular | | 2021 |
| | hyperchemokinemia in | | | | |
| | the pathogenesis of | | | | |
| | influenza | | | | |
| | G alpha (i) signaling | 0.00389 | Systemic lupus | 2.40E−07 | GWAS Catalog |
| | | | erythematosus | | 2019 |
| 5 | IGF-1 signaling | 0.0344 | Negative | 0.0031 | GO Biological Process |
| | | | regulation | | 2021 |
| | | | glucose import | | |
| | | | (GO: 0046325) | | |
| | Insulin receptor | 0.046 | Regulation of | 0.00359 | GO Biological Process |
| | signaling | | leukocyte | | 2021 |
| | | | degranulation | | |
| | | | (GO: 0043300) | | |
| | | | Positive | 0.00399 | GO Biological Process |
| | | | regulation of | | 2021 |
| | | | VEGFR | | |
| | | | signaling | | |
| | | | pathway | | |
| | | | (GO: 0030949) | | |
| | | | Corrected | 0.0023 | GWAS catalog 2019 |
| | | | insulin response | | |
| | | | Body fat | 0.019 | UK Biobank GWAS v1 |
| | | | percentage | | |
| 6 | Glycolysis I | 0.01 | Negative | 0.0022 | GO Biological Process |
| | | | regulation of | | 2021 |
| | | | autophagosome | | |
| | | | maturation | | |
| | | | (GO: 1901097) | | |
| | Agrin interactions at | 0.026 | Regulation of | 0.00538 | GO Biological Process |
| | neuromuscular | | oxidative stress- | | 2021 |
| | junction | | induced cell | | |
| | | | death | | |
| | | | (GO: 1903201) | | |
| | Maturity onset | 0.029 | Autophagosome | 0.00041 | GO Cellular |
| | diabetes of young | | (GO: 0005776) | | Component 2021 |
| | (MODY) signaling | | | | |
| | FXR/RXR activation | 0.0465 | Antihypertensive | 0.0032 | PhenGenI Association |
| | | | agents | | 2021 |
| | GABA receptor | 0.0495 | Cerebral | 0.0094 | PheWeb 2019 |
| | signaling | | aneurysm | | |
| 7 | Dilated | 0.000227 | Skeletal muscle | 0.000005 | GO Biological Process |
| | cardiomyopathy | | contraction | | 2021 |
| | signaling pathway | | (GO: 0003009) | | |
| | Calcium signaling | 0.000493 | Heart process | 0.0027 | GO Biological Process |
| | | | (GO: 0003015) | | 2021 |
| | Iron homeostasis | 0.0231 | Cardiac | 0.000999 | GO Cellular |
| | signaling pathway | | myofibril | | Component 2021 |
| | | | (GO: 0097512) | | |
| | PI3K/AKT signaling | 0.033 | Abnormal | 0.0012 | MGI Mammalian |
| | | | muscle | | Phenotype Level 4 |
| | | | relaxation | | 2021 |
| | | | (MP: 0004036) | | |
| | | | Coronary artery | 0.0000042 | Jensen Diseases |
| | | | disease | | |
| 8 | Extrinsic prothrombin | 0.0040202 | Positive | 0.000089 | GO Biological Process |
| | activation pathway | | regulation | | 2021 |
| | | | cytokine | | |
| | | | production | | |
| | | | (GO: 0001819) | | |
| | Coagulation systems | 0.00877 | Congenital | 0.0047 | PheWeb 2019 |
| | | | coagulation | | |
| | | | defects | | |
| | Intrinsic prothrombin | 0.0102 | Deep vein | 0.0071 | PheWeb 2019 |
| | activation pathway | | thrombosis | | |
| | Granulocyte adhesion | 0.0466 | Portal vein | 0.0023 | Jensen Diseases |
| | and diapedesis | | thrombosis | | |
| | NK cell signaling | 0.049 | Pulmonary | 0.005 | PhenGenI Association |
| | | | embolism | | 2021 |

Table 6: PPI-based S-LDSC results for the 46 and 67 PPI-based of genes clusters derived from SLE-associated SNPs.

TABLE 6A
SLE (GCST003155). Listed by: Name (46 PPI clusters) | Coefficient |
Coefficient_std_error | Coefficient_P_value.

Cluster5 | 1.02E−06 | 2.46E−07 | 1.55E−05; Un-clustered | 3.25E−07 | 9.82E−08
| 0.000464856; Cluster11 | 4.68E−07 | 1.55E−07 | 0.001222939; Cluster21 | 7.15E−07
| 2.49E−07 | 0.002018684; Cluster34 | 1.78E−06 | 6.33E−07 | 0.00249802;
Cluster1 | 4.77E−07 | 1.76E−07 | 0.003293693; Cluster3 | 1.02E−06 | 3.79E−07
| 0.003582676; Cluster8 | 1.22E−06 | 4.72E−07 | 0.004902907; Cluster2 | 8.69E−07
| 3.39E−07 | 0.005170708; Cluster26 | 7.14E−07 | 2.97E−07 | 0.008049906;
Cluster14 | 9.09E−07 | 3.83E−07 | 0.00885318; Cluster45 | 1.95E−06 | 8.49E−07
| 0.010658821; Cluster12 | 7.02E−07 | 3.13E−07 | 0.012474769; Cluster18 | 4.47E−07
| 2.12E−07 | 0.017622297; Cluster13 | 7.07E−07 3.41E−07 | 0.019080306;
Cluster46 | 5.50E−07 | 2.73E−07 | 0.021891815; Cluster22 | 1.66E−06 | 8.41E−07
| 0.024345886; Cluster19 | 2.38E−06 | 1.35E−06 | 0.039108149; Cluster9 | 1.41E−06
| 8.17E−07 | 0.041930849; Cluster35 | 1.85E−06 | 1.07E−06 | 0.042495651;
Cluster7 | 4.13E−07 | 2.41E−07 | 0.043271843; Cluster17 | 1.87E−07 | 1.22E−07
| 0.061615592; Cluster16 | 2.47E−07 | 1.71E−07 | 0.074136069; Cluster27 | 1.05E−06
| 7.27E−07 | 0.07482145; Cluster23 | 5.07E−07 | 4.01E−07 | 0.103265846;
Cluster39 | 9.94E−07 | 8.31E−07 | 0.115626773; Cluster37 | 3.98E−07 | 3.53E−07
| 0.1293601; Cluster40 | 2.99E−06 | 2.97E−06 | 0.156981823; Cluster32 | 4.82E−08
| 4.95E−08 | 0.165087128; Cluster30 | 1.17E−06 | 1.26E−06 | 0.175691291;
Cluster10 | 6.55E−08 | 7.89E−08 | 0.203345897; Cluster43 | 1.19E−06 | 1.53E−06
| 0.218709364; Cluster33 | 8.68E−08 1.17E−07 | 0.22991401; Cluster6 | 5.44E−07
| 7.53E−07 | 0.234917642; Cluster38 | 9.00E−07 | 1.32E−06 | 0.247844702;
Cluster29 | 3.19E−07 | 5.87E−07 | 0.293084118; Cluster4 | 2.62E−07 | 6.52E−07
| 0.344000783; Cluster42 | 3.40E−08 | 8.66E−08 | 0.34707874; Cluster41 | 1.31E−07
| 4.01E−07 | 0.372299548; Cluster25 | 7.81E−08 | 2.62E−07 | 0.382943243;
Cluster15 | 9.96E−08 | 3.71E−07 | 0.394037312; Cluster44 | 8.85E−08 | 3.81E−07
| 0.408088203; Cluster31 | −6.27E−08 | 1.29E−07 | 0.687111564; Cluster24 | −8.74E−08
| 1.15E−07 | 0.775845879; Cluster36 | −5.11E−07 | 5.86E−07 | 0.808252911; Cluster28
| −1.49E−07 | 1.54E−07 | 0.832935795;

TABLE 6B
CVD (GCST004280). Listed by: Name (46 PPI clusters) | Coefficient |
Coefficient_std_error | Coefficient_P_value.

Cluster26 | 5.46E−08 | 3.69E−08 | 0.069134216; Cluster14 | 2.41E−08 | 1.68E−08 |
0.076223927; Cluster18 | 1.65E−08 | 1.21E−08 | 0.085498551; Cluster27 | 2.32E−08 |
1.96E−08 | 0.117759489; Cluster37 | 2.89E−08 | 2.49E−08 | 0.123375531; Cluster11 |
1.16E−08 | 1.02E−08 | 0.127740675; Cluster5 | 1.12E−08 | 1.07E−08 | 0.147926858;
Cluster35 | 5.04E−08 | 5.12E−08 | 0.162327069; Cluster21 | 1.01E−08 | 1.03E−08 |
0.163088643; Cluster42 | 9.88E−09 | 1.05E−08 | 0.173316435; Cluster46 | 8.86E−09 |
9.70E−09 | 0.18051927; Cluster33 | 1.27E−08 | 1.56E−08 | 0.206678896; Cluster30 |
1.10E−08 | 1.37E−08 | 0.211357056; Cluster23 | 2.77E−08 | 3.68E−08 | 0.225923123;
Cluster7 | 1.97E−08 | 3.46E−08 | 0.28415359; Cluster38 | 4.08E−08 | 7.92E−08 |
0.303281179; Cluster32 | 1.44E−09 | 2.80E−09 | 0.304146619; Cluster31 | 6.78E−09 |
1.40E−08 | 0.313969568; Cluster13 | 8.84E−09 | 1.85E−08 | 0.316145582; Cluster16 |
3.18E−09 | 7.88E−09 | 0.343328263; Cluster34 | 7.44E−09 | 1.93E−08 | 0.349624817;
Cluster19 | 2.92E−08 | 1.14E−07 | 0.398747125; Cluster28 | 3.22E−09 | 1.75E−08 |
0.426892776; Cluster10 | 1.19E−09 | 6.54E−09 | 0.427566727; Cluster40 | 1.72E−08 |
1.31E−07 | 0.447882877; Cluster12 | 5.16E−10 | 1.03E−08 | 0.480019977; Cluster43 |
1.11E−09 | 3.89E−08 | 0.488633214; Cluster36 | 3.13E−09 | 1.21E−07 | 0.489628178;
Cluster4 | −6.90E−09 | 1.12E−07 | 0.524577162; Cluster15 | −3.65E−09 | 3.44E−08 |
0.542262626; Cluster8 | −2.54E−09 | 1.10E−08 | 0.591005995; Cluster6 | −8.61E−09 |
3.66E−08 | 0.593050962; Cluster41 | −6.75E−09 2.77E−08 | 0.596321433; Un-clustered |
−1.23E−09 | 3.95E−09 | 0.622122863; Cluster39 | −1.67E−08 | 2.91E−08 | 0.717427638;
Cluster17 | −8.51E−09 | 7.85E−09 | 0.860711443; Cluster1 −8.57E−09 | 7.83E−09 |
0.863219649; Cluster29 | −1.25E−08 | 1.08E−08 | 0.874930281; Cluster45 | −2.82E−08 |
2.01E−08 | 0.919958865; Cluster2 | −1.17E−08 | 6.62E−09 | 0.961109206; Cluster3
| −1.08E−08 | 4.48E−09 | 0.992107172; Cluster44 | −9.98E−09 | 4.11E−09 | 0.992425412;
Cluster24 | −2.99E−08 | 1.12E−08 | 0.996174967; Cluster25 | −2.28E−08 | 8.06E−09 |
0.997622064; Cluster22 | −3.68E−08 | 1.30E−08 | 0.997706133; Cluster9 | −3.48E−08 |
5.04E−09 | 1;

TABLE 6C
CAD-I (GCST000998). Listed by: Name (46 PPI clusters) | Coefficient |
Coefficient_std_error | Coefficient_P_value.

Cluster45 | 7.72E−08 | 3.78E−08 | 0.020686598; Cluster26 | 8.22E−08 | 4.73E−08
| 0.040922778; Cluster5 | 1.72E−08 | 1.47E−08 | 0.120106703; Cluster31 | 7.22E−08
| 6.62E−08 | 0.137591978; Cluster14 | 2.45E−08 | 2.28E−08 | 0.141338714; Un-
clustered | 7.81E−09 | 8.22E−09 | 0.17091628; Cluster32 | 2.26E−08 | 2.54E−08
| 0.186803017; Cluster46 | 1.40E−08 | 1.58E−08 | 0.187226904; Cluster42 | 1.07E−08
| 1.28E−08 | 0.200422846; Cluster13 | 3.22E−08 | 3.88E−08 | 0.20332734;
Cluster8 | 2.36E−08 | 2.93E−08 | 0.20982953; Cluster18 | 1.21E−08 | 1.71E−08
| 0.240889076; Cluster30 | 3.86E−08 | 5.99E−08 | 0.259756644; Cluster29 | 4.04E−08
| 6.34E−08 | 0.26230149; Cluster33 | 1.09E−08 | 1.74E−08 | 0.266616626;
Cluster44 | 1.24E−08 | 2.53E−08 | 0.312205605; Cluster25 | 1.18E−08 | 2.91E−08
| 0.342394886; Cluster35 | 1.81E−08 | 4.66E−08 | 0.348753129; Cluster12 | 7.00E−09
| 1.88E−08 | 0.354943023; Cluster38 | 1.91E−08 | 5.40E−08 | 0.361714503;
Cluster9 | 1.02E−08 | 3.47E−08 | 0.383733554; Cluster3 | 3.69E−09 | 1.78E−08
| 0.418104799; Cluster27 | 9.10E−09 | 6.94E−08 | 0.447844612; Cluster34 | 3.42E−09
| 4.22E−08 | 0.467701692; Cluster41 | 1.04E−09 | −7.69E−08 | 0.494602616; Cluster28
| −4.50E−09 | 4.68E−08 | 0.538296904; Cluster4 | −1.20E−08 | 8.19E−08 | 0.558146649;
Cluster43 | −2.81E−08 | 8.33E−08 | 0.631847254; Cluster37 | −1.95E−08 | 4.64E−08
| 0.662452883; Cluster22 | −2.03E−08 | 3.87E−08 | 0.700560595; Cluster17 | −1.05E−08
| 1.99E−08 | 0.701426077; Cluster2 | −1.17E−08 | 1.67E−08 | 0.7580151; Cluster6
| −1.94E−08 | 2.37E−08 | 0.793355339; Cluster24 | −2.28E−08 | 2.61E−08 | 0.808874907;
Cluster16 | −1.36E−08 | 1.42E−08 | 0.830439622; Cluster11 | −9.24E−09 | 9.58E−09
| 0.832769023; Cluster40 | −3.12E−08 | 2.89E−08 | 0.859252419; Cluster7 | −2.57E−08
| 2.07E−08 | 0.893537621; Cluster21 | −1.39E−08 | 1.05E−08 | 0.908094544; Cluster23
| −5.01E−08 | 3.58E−08 | 0.919632909; Cluster15 | −3.70E−08 | 1.65E−08 | 0.987427533;
Cluster1 | −2.29E−08 | 1.01E−08 | 0.988147189; Cluster36 | −1.46E−07 | 6.07E−08
| 0.991931986; Cluster10 | −2.60E−08 | 1.08E−08 | 0.992008673; Cluster19 | −7.79E−08
1.20E−08 | 1; Cluster39 | −6.95E−08 | 8.05E−09 | 1;

TABLE 6D
CAD-II (GCST001479). Listed by: Name (46 PPI clusters)
| Coefficient | Coefficient_std_error |
Coefficient_P_value.

Cluster5 | 7.81E−08 | 3.50E−08 | 0.012748573; Cluster26 8.63E−08 | 4.08E−08
| 0.017291548; Cluster31 | 2.33E−07 | 1.53E−07 | 0.064315068; Cluster33 | 3.64E−08
| 3.42E−08 | 0.143486275; Cluster14 | 4.80E−08 | 5.17E−08 | 0.17632669;
Cluster42 | 2.82E−08 | 3.08E−08 | 0.180378478; Cluster8 | 4.28E−08 | 7.67E−08
| 0.288682068; Cluster29 | 6.20E−08 | 1.21E−07 | 0.303719152; Cluster32 | 1.00E−08
| 2.46E−08 | 0.341554255; Cluster35 | 3.91E−08 | 9.79E−08 | 0.344669861;
Cluster17 | 2.89E−08 | 7.45E−08 | 0.349116393; Cluster37 | 2.01E−08 | 6.32E−08
| 0.375032789; Un-clustered | 2.95E−09 | 1.60E−08 | 0.426659847; Cluster13 | 9.41E−10
| 2.93E−08 | 0.487190105; Cluster21 | −1.83E−09 | 2.08E−08 | 0.534988649; Cluster1
| −9.29E−09 | 6.19E−08 | 0.559694408; Cluster27 | −1.96E−08 | 1.05E−07 | 0.573905343;
Cluster30 −1.57E−08 | 6.56E−08 | 0.594706559; Cluster38 | −1.30E−08 | 5.36E−08
| 0.595568721; Cluster3 | −9.41E−09 | 3.38E−08 | 0.609806329; Cluster6 | −3.19E−08
| 9.59E−08 | 0.630393297; Cluster15 | −3.36E−08 | 1.00E−07 | 0.6310239; Cluster34
| −1.98E−08 | 5.01E−08 | 0.653406768; Cluster16 | −1.42E−08 | 3.19E−08 | 0.672175904;
Cluster43 −3.86E−08 | 5.93E−08 | 0.742486229; Cluster44 −2.60E−08 | 3.50E−08
| 0.77102706; Cluster45 | −6.69E−08 | 8.86E−08 | 0.774931551; Cluster4 | −5.46E−08
| 6.73E−08 | 0.791087188; Cluster12 | −2.12E−08 | 2.45E−08 | 0.806273864; Cluster41
| −5.27E−08 | 5.98E−08 | 0.811091068; Cluster19 | −8.11E−08 | 8.83E−08 | 0.820688925;
Cluster46 | −2.06E−08 | 2.12E−08 | 0.83500636; Cluster7 | −3.28E−08 | 3.30E−08
| 0.839662314; Cluster11 | −1.83E−08 | 1.43E−08 | 0.899579234; Cluster25 | −4.16E−08
| 3.24E−08 | 0.900038516; Cluster9 | −5.79E−08 | 4.33E−08 | 0.909490132; Cluster18
| −2.60E−08 | 1.72E−08 | 0.934488339; Cluster24 | −7.63E−08 | 4.97E−08 | 0.93762274;
Cluster28 | 7.50E−08 | 4.86E−08 | 0.938767911; Cluster10 | −2.17E−08 | 1.29E−08
| 0.954059057; Cluster36 | −1.36E−07 | 5.96E−08 | 0.988493568; Cluster2 | −4.98E−08
| 2.10E−08 | 0.991005078; Cluster23 | −1.40E−07 | 4.62E−08 | 0.998753736; Cluster22
| −1.19E−07 | 3.81E−08 | 0.999083217; Cluster40 | −1.24E−07 | 2.92E−08 | 0.99998855;
Cluster39 −1.23E−07 | 2.20E−08 | 0.999999988;

TABLE 6E
SLE (GCST003155). Listed by: Name (67 PPI clusters) | Coefficient |
Coefficient_std_error | Coefficient_P_value.

Cluster16 | 2.28E−06 | 4.67E−07 | 5.37E−07; Cluster19 | 3.04E−07 | 7.74E−08 | 4.22E−05;
Un-clustered | 3.58E−07 | 9.40E−08 | 7.01E−05; Cluster1 | 5.15E−07 | 1.63E−07
| 0.00080553; Cluster2 | 5.28E−07 | 1.98E−07 | 0.003769081; Cluster9 | 6.84E−07
| 2.66E−07 | 0.005063228; Cluster4 | 4.18E−07 | 1.93E−07 | 0.015053972;
Cluster5 | 4.86E−07 | 2.35E−07 | 0.019309256; Cluster30 | 9.30E−07 | 4.53E−07
| 0.020177563; Cluster12 | 4.77E−07 | 2.37E−07 | 0.02205929; Cluster55 | 5.01E−07
| 2.57E−07 | 0.025773553; Cluster56 | 5.88E−07 | 3.09E−07 | 0.028258295;
Cluster43 | 4.02E−07 | 2.34E−07 | 0.042449316; Cluster38 | 3.32E−07 | 1.94E−07
| 0.043625565; Cluster32 | 8.93E−07 | 5.25E−07 | 0.044372624; Cluster8 | 1.50E−06
| 8.93E−07 | 0.046393504; Cluster17 | 1.06E−06 | 6.40E−07 | 0.048337948;
Cluster46 | 9.24E−07 | 5.73E−07 | 0.053394907; Cluster35 | 6.50E−07 | 4.12E−07
| 0.05721673; Cluster47 | 5.26E−07 | 3.43E−07 | 0.062473331; Cluster3 | 2.29E−06
| 1.56E−06 | 0.071906321; Cluster6 | 5.96E−07 | 4.32E−07 | 0.08371441;
Cluster21 | 1.84E−07 | 1.33E−07 | 0.084145482; Cluster61 | 6.49E−07 | 4.76E−07
| 0.086714162; Cluster18 | 3.92E−07 | 2.97E−07 | 0.093187887; Cluster7 | 4.01E−07
| 3.08E−07 | 0.096391712; Cluster63 | 3.50E−07 | 2.70E−07 | 0.097461384;
Cluster57 | 3.23E−07 | 2.53E−07 | 0.101376592; Cluster67 | 2.91E−06 | 2.29E−06
| 0.101557677; Cluster34 | 7.76E−07 | 6.50E−07 | 0.116174683; Cluster23 | 9.75E−07
| 8.63E−07 | 0.129453311; Cluster48 | 2.92E−07 | 2.63E−07 | 0.133534512;
Cluster62 | 3.94E−07 | 3.56E−07 | 0.134157872; Cluster53 | 5.51E−07 | 5.24E−07
| 0.146759644; Cluster11 | 3.54E−07 | 3.40E−07 | 0.149038935; Cluster15 | 3.52E−07
| 3.38E−07 | 0.149248354; Cluster10 | 3.48E−07 | 3.38E−07 | 0.152012892;
Cluster64 | 1.27E−06 | 1.25E−06 | 0.15472143; Cluster44 | 2.09E−07 | 2.17E−07
| 0.167129509; Cluster54 | 1.96E−07 | 2.16E−07 | 0.182344402; Cluster45 | 1.28E−06
| 1.46E−06 | 0.191390031; Cluster66 | 2.04E−07 | 2.36E−07 | 0.193517921;
Cluster20 | 1.39E−07 | 1.69E−07 | 0.20590916; Cluster14 | 6.27E−08 | 8.01E−08
| 0.21676885; Cluster49 | 9.79E−08 | 1.32E−07 | 0.229148758; Cluster50 | 8.97E−07
| 1.24E−06 | 0.234853793; Cluster58 | 2.08E−07 | 3.12E−07 | 0.252587711;
Cluster40 | 2.10E−07 | 3.31E−07 | 0.262568522; Cluster25 | 8.74E−08 | 1.38E−07
| 0.263684508; Cluster39 | 2.16E−07 | 3.51E−07 | 0.269136405; Cluster52 | 3.51E−07
| 5.78E−07 | 0.271745731; Cluster33 | 1.20E−07 | 2.10E−07 | 0.283690098;
Cluster24 | 7.75E−07 | 1.37E−06 | 0.28597907; Cluster60 | 4.41E−08 | 7.82E−08
| 0.286417164; Cluster31 | 1.23E−07 | 2.34E−07 | 0.299648943; Cluster28 | 4.76E−08
| 1.03E−07 | 0.322063574; Cluster65 | 8.61E−08 | 3.78E−07 | 0.409981456;
Cluster51 | 4.72E−08 | 3.32E−07 | 0.443474656; Cluster29 | 1.58E−08 | 2.08E−07
| 0.469754614; Cluster42 | 6.07E−09 | 1.01E−07 | 0.47605828; Cluster13 | −1.88E−09
| 1.14E−07 | 0.506591258; Cluster22 −6.86E−09 | 2.24E−07 | 0.512190558; Cluster27
| −8.42E−09 | 1.29E−07 | 0.526100499; Cluster59 | −1.03E−07 | 3.04E−07 | 0.632811921;
Cluster36 | −3.74E−08 | 6.33E−08 | 0.722821081; Cluster41 | −9.47E−08 | 1.17E−07
| 0.79051551;

TABLE 6F
CVD (GCST004280). Listed by: Name (67 PPI clusters) | Coefficient |
Coefficient_std_error | Coefficient_P_value.

Cluster33 | 4.07E−08 | 1.90E−08 | 1.60E−02; Cluster39 | 8.07E−08 | 3.96E−08 | 2.07E−02;
Cluster46 | 2.24E−08 | 1.12E−08 | 2.27E−02; Cluster61 | 3.10E−08 | 1.66E−08 | 0.031000116;
Cluster10 | 2.50E−08 | 1.42E−08 | 0.038895206; Cluster31 | 3.30E−08 | 2.13E−08
| 0.060230458; Cluster18 | 2.04E−08 | 1.57E−08 | 0.097553505; Cluster38 | 3.56E−08
| 2.83E−08 | 0.104329761; Cluster20 | 2.02E−08 | 1.74E−08 | 0.123683623;
Cluster22 | 8.01E−08 | 7.01E−08 | 0.126658314; Cluster9 | 1.50E−08 | 1.32E−08
| 0.127883591; Cluster56 | 2.17E−08 | 2.46E−08 | 0.188934422; Cluster57 | 1.46E−08
| 1.72E−08 | 0.198249459; Cluster63 | 1.95E−08 | 2.60E−08 | 0.226705071;
Cluster47 | 1.58E−08 | 2.40E−08 | 0.254598807; Cluster40 | 2.25E−08 | 3.53E−08
| 0.261745637; Cluster21 | 5.70E−09 | 9.60E−09 | 0.276407181; Cluster29 | 1.02E−08
| 1.75E−08 | 0.280102205; Cluster28 | 6.91E−09 | 1.22E−08 | 0.286250222;
Cluster44 | 2.66E−08 | 4.72E−08 | 0.28661858; Cluster14 | 4.71E−09 | 8.45E−09
| 0.28869697; Cluster19 | 2.19E−09 | 4.46E−09 | 0.311930588; Cluster11 | 1.15E−08
| 2.46E−08 | 0.319592653; Cluster5 | 4.95E−09 | 1.21E−08 | 0.34145471;
Cluster13 | 4.74E−09 | 1.31E−08 | 0.358993461; Cluster50 | 1.47E−08 | 4.10E−08
| 0.359761001; Cluster23 | 4.94E−09 | 1.44E−08 | 0.366130912; Cluster67 | 7.67E−09
| 3.05E−08 | 0.400892026; Cluster27 | 1.49E−09 | 7.93E−09 | 0.425222116;
Cluster16 | 2.67E−09 | 1.50E−08 | 0.429651493; Cluster15 | 7.62E−09 | 4.30E−08
| 0.429697171; Cluster4 | 5.91E−10 | 5.64E−09 | 0.458265048; Un-clustered | 3.04E−11
| 3.78E−09 | 0.496795343; Cluster32 | −2.88E−10 | 1.08E−08 | 0.510686482; Cluster8
| −2.81E−10 | 9.01E−09 | 0.512445298; Cluster66 | −1.34E−09 | 1.79E−08 | 0.529772918;
Cluster35 | −1.25E−09 | 9.07E−09 | 0.554623425; Cluster43 | −2.19E−09 | 9.88E−09
| 0.587547047; Cluster48 | −1.58E−08 | 4.07E−08 | 0.651447232; Cluster1 | −2.85E−09
| 7.18E−09 | 0.654429728; Cluster45 | −6.54E−09 | 1.21E−08 | 0.705647081; Cluster62
| −1.69E−08 | 2.47E−08 | 0.753321648; Cluster59 | −9.49E−09 | 1.34E−08 | 0.761498691;
Cluster52 | −9.07E−09 | 1.00E−08 | 0.817027717; Cluster24 | −1.70E−08 | 1.85E−08
| 0.820998615; Cluster53 | −1.07E−08 | 1.12E−08 | 0.829105921; Cluster54 | −1.06E−08
| 1.02E−08 | 0.850536589; Cluster55 | −1.32E−08 | 1.27E−08 | 0.852028244; Cluster7
| −8.56E−09 | 7.80E−09 | 0.863841543; Cluster6 | −1.15E−08 | 1.01E−08 | 0.873193548;
Cluster60 | −1.48E−08 | 1.26E−08 | 0.87991001; Cluster2 | −8.48E−09 | 6.00E−09
| 0.921271569; Cluster64 | −1.94E−08 | 1.33E−08 | 0.928476456; Cluster12 | −1.06E−08
| 7.00E−09 | 0.935977064; Cluster17 | −1.65E−08 | 1.05E−08 | 0.941518308; Cluster36
| −4.35E−09 | 2.63E−09 | 0.950803579; Cluster34 | −1.44E−08 | 8.57E−09 | 0.953879937;
Cluster51 | −2.38E−08 | 1.37E−08 | 0.95920167; Cluster49 | −1.39E−08 | 7.11E−09
| 0.974548492; Cluster58 | −2.48E−08 | 1.26E−08 | 0.975751652; Cluster42 | −1.52E−08
| 7.68E−09 | 0.975826579; Cluster25 | −8.86E−09 | 3.98E−09 | 0.986897247; Cluster41
| −1.65E−08 | 7.25E−09 | 0.988515127; Cluster65 | −9.66E−09 | 4.04E−09 | 0.99165235;
Cluster30 | −2.17E−08 | 7.28E−09 | 0.998570603; Cluster3 | −2.69E−08 | 8.08E−09
| 0.999555462;

TABLE 6G
CAD-I (GCST000998). Listed by: Name (67 PPI clusters) | Coefficient |
Coefficient_std_error | Coefficient_P_value.

Cluster11 | 5.37E−08 | 2.70E−08 | 2.34E−02; Cluster12 | 3.84E−08 | 2.31E−08 | 4.84E−02;
Cluster35 | 4.25E−08 | 2.81E−08 | 6.50E−02; Cluster18 | 7.88E−08 | 5.65E−08 | 0.081502522;
Cluster16 | 3.35E−08 | 2.63E−08 | 0.101087078; Cluster19 | 1.10E−08 | 9.52E−09
| 0.124807886; Cluster38 | 4.18E−08 | 3.65E−08 | 0.126042359; Cluster62 | 5.02E−08
| 4.79E−08 | 0.147356556; Cluster67 | 8.38E−08 | 8.41E−08 | 0.159572869;
Cluster39 | 9.53E−08 | 9.73E−08 | 0.163901517; Cluster3 | 5.07E−08 | 5.39E−08
| 0.173251683; Cluster53 | 7.27E−08 | 8.82E−08 | 0.204833521; Cluster54 | 2.66E−08
| 3.37E−08 | 0.214895573; Cluster64 | 3.38E−08 | 4.44E−08 | 0.22327849;
Cluster17 | 1.84E−08 | 2.67E−08 | 0.245515294; Cluster5 | 9.37E−09 | 1.56E−08
| 0.273663755; Cluster49 | 1.29E−08 | 2.34E−08 | 0.29142778; Cluster23 | 4.76E−08
| 8.76E−08 | 0.293532748; Cluster36 | 6.43E−09 | 1.24E−08 | 0.302011325;
Cluster55 | 1.24E−08 | 2.42E−08 | 0.304552635; Cluster51 | 2.72E−08 | 5.48E−08
| 0.309968603; Cluster60 | 2.51E−08 | 5.26E−08 | 0.316680947; Cluster65 | 1.18E−08
| 2.51E−08 | 0.318957667; Cluster14 | 5.67E−09 | 1.38E−08 | 0.340143467;
Cluster29 | 1.50E−08 | 3.68E−08 | 0.341746599; Cluster34 | 9.37E−09 | 2.46E−08
0.351882514; Cluster27 | 4.90E−09 | 2.01E−08 | 0.403759082; Cluster28 | 2.95E−09
| 1.44E−08 | 0.418764255; Cluster45 | 4.08E−09 | 2.02E−08 | 0.419959337;
Cluster46 | 1.50E−08 | 8.26E−08 | 0.427934767; Cluster25 | 1.91E−09 | 1.19E−08
| 0.436179135; Un-clustered | 1.28E−09 | 8.12E−09 | 0.437370496; Cluster22 | 6.81E−09
| 4.38E−08 | 0.438271118; Cluster4 | 2.60E−09 | 1.92E−08 | 0.446103183;
Cluster8 | 2.38E−09 | 3.78E−08 | 0.474912118; Cluster41 | 1.55E−09 | 2.59E−08
| 0.476064826; Cluster9 | −1.18E−10 | 1.63E−08 | 0.502892978; Cluster44 | −1.31E−08
| 7.93E−08 | 0.565522544; Cluster47 | −6.95E−09 | 2.74E−08 | 0.600154698; Cluster58
| −1.10E−08 | 4.01E−08 | 0.607674566; Cluster56 | −8.04E−09 | 2.52E−08 | 0.625053085;
Cluster6 | −9.32E−09 | 2.83E−08 | 0.629082086; Cluster7 | −4.54E−09 | 1.29E−08
| 0.637634827; Cluster33 | −8.45E−09 | 2.31E−08 | 0.642846508; Cluster31 | −1.10E−08
| 2.72E−08 | 0.657418129; Cluster30 | −1.02E−08 | 2.45E−08 | 0.661148804; Cluster57
| −1.85E−08 | 3.71E−08 | 0.691116369; Cluster42 | −1.00E−08 | 2.01E−08 | 0.691184872;
Cluster1 | −8.13E−09 | 1.31E−08 | 0.732721896; Cluster2 | −8.82E−09 | 1.30E−08
| 0.75132722; Cluster10 | −1.22E−08 | 1.79E−08 | 0.752930833; Cluster66 | −1.81E−08
| 2.40E−08 | 0.774163729; Cluster43 | −1.41E−08 | 1.37E−08 | 0.847264613; Cluster52
| −2.96E−08 | 2.89E−08 | 0.847810714; Cluster63 | −2.73E−08 | 2.62E−08 | 0.85081754;
Cluster59 | −2.41E−08 | 2.13E−08 | 0.871153306; Cluster15 | −3.04E−08 | 2.55E−08
| 0.883092265; Cluster13 | −1.97E−08 | 1.62E−08 0.888699573; Cluster40 | −4.28E−08
| 3.41E−08 | 0.895186993; Cluster32 | −3.56E−08 | 2.78E−08 | 0.899966105; Cluster20
| −2.90E−08 | 1.88E−08 | 0.938878832; Cluster21 | −2.36E−08 | 1.04E−08 | 0.988280991;
Cluster50 | −3.32E−08 | 1.37E−08 | 0.992291883; Cluster48 | −5.71E−08 | 2.23E−08
| 0.994866268; Cluster61 | −3.20E−08 | 8.63E−09 | 0.999894192; Cluster24 | −5.29E−08
| 1.23E−08 | 0.999991244;

TABLE 6H
CAD-II (GCST001479). Listed by: Name (67 PPI clusters)
| Coefficient | Coefficient_std_error |
Coefficient_P_value.

Cluster39 | 2.05E−07 | 1.03E−07 | 2.33E−02; Cluster10 | 4.42E−08 | 3.11E−08 | 7.75E−02;
Cluster14 | 4.16E−08 | 3.29E−08 | 1.03E−01; Cluster3 | 1.79E−07 | 1.66E−07 | 0.139507422;
Cluster60 | 2.95E−08 | 2.82E−08 | 0.148235941; Cluster18 | 6.42E−08 | 6.25E−08
| 0.1518846; Cluster27 | 3.28E−08 | 3.23E−08 | 0.154993216; Cluster19 | 1.39E−08
| 1.39E−08 | 0.158152181; Cluster47 | 5.57E−08 | 5.67E−08 | 0.162705472;
Cluster38 | 5.72E−08 | 5.98E−08 | 0.169502773; Cluster23 | 7.61E−08 | 8.59E−08
| 0.187862221; Cluster34 | 5.31E−08 | 6.15E−08 | 0.194186026; Cluster16 | 3.85E−08
| 4.82E−08 | 0.21249692; Cluster53 | 1.24E−07 | 1.70E−07 | 0.232665633;
Cluster28 | 2.10E−08 | 2.98E−08 | 0.24012461; Cluster29 | 5.81E−08 | 8.73E−08
| 0.253003641; Cluster31 | 2.28E−08 | 3.84E−08 | 0.276466429; Cluster4 | 2.09E−08
| 3.58E−08 | 0.279257814; Cluster1 | 3.26E−08 | 5.73E−08 | 0.284530364;
Cluster9 | 1.44E−08 | 2.71E−08 | 0.297217966; Cluster57 | 3.39E−08 | 7.14E−08
| 0.317521353; Cluster67 | 2.65E−08 | 7.84E−08 | 0.367778853; Cluster48 | 1.30E−07
| 3.86E−07 | 0.368280586; Cluster64 | 2.24E−08 | 7.69E−08 | 0.385518862;
Cluster62 | 1.22E−08 | 5.40E−08 | 0.410790692; Cluster63 | 9.74E−09 | 5.31E−08
| 0.42719768; Cluster35 | 5.17E−09 | 2.92E−08 | 0.429796987; Cluster5 | 3.16E−09
| 2.60E−08 | 0.451546779; Cluster46 | 1.22E−08 | 1.35E−07 | 0.463896722; Cluster11
| −3.26E−09 | 3.50E−08 | 0.537047091; Cluster7 | −5.55E−09 | 2.11E−08 | 0.6036065;
Cluster12 | −6.35E−09 | 2.40E−08 | 0.604405699; Cluster15 | −3.41E−08 | 9.15E−08
| 0.645362679; Cluster45 | −1.91E−08 | 4.75E−08 | 0.656315302; Cluster36 | −1.03E−08
| 2.38E−08 | 0.66678841; Cluster54 | −1.46E−08 | 2.46E−08 | 0.723828521; Cluster6
| −3.24E−08 | 5.26E−08 | 0.730943707; Cluster8 | −2.92E−08 | 3.70E−08 | 0.784453374;
Cluster21 | −1.66E−08 | 2.09E−08 | 0.785801134; Cluster66 | −4.76E−08 | 5.73E−08
| 0.796778552; Cluster65 | −2.89E−08 | 3.42E−08 | 0.801332254; Cluster49 | −4.21E−08
| 4.81E−08 | 0.809183044; Cluster41 | −2.96E−08 | 3.29E−08 | 0.816227241; Cluster25
| −1.76E−08 | 1.85E−08 | 0.828998139; Cluster2 | −2.44E−08 | 2.36E−08 | 0.850036369;
Cluster51 | −2.01E−08 | 1.90E−08 | 0.854803679; Un-clustered | −1.88E−08 | 1.76E−08
| 0.857574864; Cluster32 | −4.35E−08 | 3.92E−08 | 0.866698229; Cluster22 | −7.30E−08
| 5.26E−08 | 0.917377177; Cluster52 −3.71E−08 | 2.67E−08 | 0.917467039; Cluster42
| −3.72E−08 | 2.50E−08 | 0.93184256; Cluster59 | −6.24E−08 | 3.44E−08 | 0.965167779;
Cluster55 | −4.19E−08 | 2.31E−08 | 0.965590288; Cluster20 | −3.91E−08 | 2.10E−08
| 0.968459473; Cluster56 | −3.43E−08 | 1.80E−08 | 0.971407936; Cluster50 | −7.76E−08
| 4.08E−08 | 0.971503519; Cluster33 | −5.97E−08 | 3.06E−08 | 0.974296428; Cluster44
| −9.71E−08 | 4.52E−08 | 0.984141623; Cluster13 | −3.24E−08 | 1.48E−08 | 0.985542582;
Cluster43 | −4.38E−08 | 1.58E−08 | 0.997267523; Cluster17 | −7.28E−08 | 2.13E−08
| 0.999686316; Cluster61 | −4.97E−08 | 1.44E−08 | 0.999721095; Cluster30 | −9.95E−08
| 2.70E−08 | 0.999882433; Cluster40 | −1.26E−07 | 3.32E−08 | 0.999921373; Cluster58
| −9.32E−08 | 2.31E−08 | 0.999973294; Cluster24 | −7.86E−08 | 1.68E−08 | 0.999998464;

Table 7: Validation of PPI-based MR-IVW results for 46 and 67 SLE-derived clusters on CAD. MR-IVW results for SLE on CVD-related summary statistics available on the MR-base platform, including two CAD GWAS, two MI GWAS, Ischemic Stroke, Cardiomyopathy, and Atrial Fibrillation.

TABLE 7A
46 PPI-based cluser MR analysis (FIG. 10A). Listed by: Cluster
| Exposure_data | number of SNPs linking to each
cluster | Outcome | MR method | # SNPs used as IVs | beta | SE | pval

1 | SLE GWAS | 130 | Atrial fibrillation || id: ebi-a-GCST006414 | Inverse variance
weighted | 113 | −0.013091965 | 0.006556942 | 0.04586245; 2 | SLE GWAS | 111 | Atrial
fibrillation || id: ebi-a-GCST006414 | Inverse variance
weighted | 100 | 0.006822748 | 0.004654236 | 0.142669472; 3 | SLE GWAS | 175 | Atrial
fibrillation || id: ebi-a-GCST006414 | Inverse variance
weighted | 147 | 0.007840831 | 0.002707883 | 0.003784853; 4 | SLE GWAS | 30 | Atrial
fibrillation || id: ebi-a-GCST006414 | Inverse variance
weighted | 28 | 0.011712407 | 0.005879447 | 0.046360816; 5 | SLE GWAS | 383 | Atrial
fibrillation || id: ebi-a-GCST006414 | Inverse variance weighted | 332
| −0.015489275 | 0.004368884 | 0.000392075; 6 | SLE GWAS | 5 | Atrial fibrillation || id: ebi-
a-GCST006414 | Inverse variance weighted | 4
| −0.031709677 | 0.046556439 | 0.495807044; 7 | SLE GWAS | 39 | Atrial fibrillation || id: ebi-
a-GCST006414 | Inverse variance weighted | 33
| −0.034354152 | 0.01706029 | 0.044041984; 8 | SLE GWAS | 67 | Atrial fibrillation || id: ebi-
a-GCST006414 | Inverse variance weighted | 57
| −0.009585706 | 0.009325365 | 0.303988609; 9 | SLE GWAS | 23 | Atrial fibrillation || id: ebi-
a-GCST006414 | Inverse variance weighted | 18
| −0.004122902 | 0.017952651 | 0.818360474; 10 | SLE GWAS | 99 | Atrial fibrillation ||
id: ebi-a-GCST006414 | Inverse variance
weighted | 89 | 0.011817879 | 0.009427226 | 0.209990971; 11 | SLE GWAS | 183 | Atrial
fibrillation || id: ebi-a-GCST006414 | Inverse variance
weighted | 154 | 0.015715054 | 0.006553889 | 0.016492913; 12 | SLE GWAS | 199 | Atrial
fibrillation || id: ebi-a-GCST006414 | Inverse variance weighted | 165
| −0.049680473 | 0.00577268 | 7.55639E−18; 13 | SLE GWAS | 55 | Atrial fibrillation || id: ebi-
a-GCST006414 | Inverse variance
weighted | 49 | 0.013866957 | 0.005934009 | 0.019446378; 14 | SLE GWAS | 173 | Atrial
fibrillation || id: ebi-a-GCST006414 | Inverse variance
weighted | 155 | 0.010317619 | 0.002918067 | 0.000406587; 15 | SLE GWAS | 3 | Atrial
fibrillation || id: ebi-a-GCST006414 | Inverse variance weighted | 2
| −0.256128667 | 0.084230969 | 0.002359585; 16 | SLE GWAS | 53 | Atrial fibrillation ||
id: ebi-a-GCST006414 | Inverse variance
weighted | 45 | 0.012345978 | 0.006163898 | 0.045182696; 17 | SLE GWAS | 40 | Atrial
fibrillation || id: ebi-a-GCST006414 | Inverse variance weighted | 31
| −0.039305108 | 0.025983815 | 0.130361838; 18 | SLE GWAS | 196 | Atrial fibrillation ||
id: ebi-a-GCST006414 | Inverse variance weighted | 174
| −0.03825668 | 0.005481635 | 2.97154E−12; 19 | SLE GWAS | 24 | Atrial fibrillation || id: ebi-
a-GCST006414 | Inverse variance weighted | 19 | −0.14705925 | 0.011484658 | 1.54036E−37;
20 | SLE GWAS | 2 | Atrial fibrillation || id: ebi-a-GCST006414 | Inverse variance
weighted | 2 | 0.031328074 | 0.34857976 | 0.92838776; 21 | SLE GWAS | 222 | Atrial
fibrillation || id: ebi-a-GCST006414 | Inverse variance
weighted | 196 | 0.008217342 | 0.002890928 | 0.004476712; 22 | SLE GWAS | 48 | Atrial
fibrillation || id: ebi-a-GCST006414 | Inverse variance weighted | 42
| −0.016947532 | 0.006679849 | 0.011177086; 23 | SLE GWAS | 12 | Atrial fibrillation ||
id: ebi-a-GCST006414 | Inverse variance
weighted | 12 | 0.011277944 | 0.017899867 | 0.52865701; 24 | SLE GWAS | 17 | Atrial
fibrillation || id: ebi-a-GCST006414 | Inverse variance
weighted | 14 | 0.029702727 | 0.027904697 | 0.287131834; 25 | SLE GWAS | 10 | Atrial
fibrillation || id: ebi-a-GCST006414 | Inverse variance
weighted | 7 | 0.107276339 | 0.167533474 | 0.521959517; 26 | SLE GWAS | 38 | Atrial
fibrillation || id: ebi-a-GCST006414 | Inverse variance weighted | 30
| −0.033198821 | 0.014197787 | 0.019371201; 27 | SLE GWAS | 9 | Atrial fibrillation || id: ebi-
a-GCST006414 | Inverse variance
weighted | 7 | 0.017349879 | 0.048326757 | 0.719586216; 28 | SLE GWAS | 11 | Atrial
fibrillation || id: ebi-a-GCST006414 | Inverse variance weighted | 8
| −0.008731649 | 0.034166005 | 0.79828642; 29 | SLE GWAS | 15 | Atrial fibrillation || id: ebi-
a-GCST006414 | Inverse variance
weighted | 13 | 0.025538806 | 0.024474352 | 0.296720185; 30 | SLE GWAS | 10 | Atrial
fibrillation || id: ebi-a-GCST006414 | Inverse variance weighted | 6
| −0.085624247 | 0.051275456 | 0.094941613; 31 | SLE GWAS | 14 | Atrial fibrillation ||
id: ebi-a-GCST006414 | Inverse variance weighted | 11
| −0.024215578 | 0.030001437 | 0.419581646; 32 | SLE GWAS | 5 | Atrial fibrillation || id: ebi-
a-GCST006414 | Inverse variance weighted | 4 | 0.157952232 | 0.10330805 | 0.126278304;
33 | SLE GWAS | 51 | Atrial fibrillation || id: ebi-a-GCST006414 | Inverse variance
weighted | 45 | 0.063059767 | 0.01537715 | 4.12E−05; 34 | SLE GWAS | 31 | Atrial
fibrillation || id: ebi-a-GCST006414 | Inverse variance weighted | 25
| −0.115334857 | 0.01653756 | 3.07801E−12; 35 | SLE GWAS | 19 | Atrial fibrillation || id: ebi-
a-GCST006414 | Inverse variance
weighted | 16 | 0.001910269 | 0.021620849 | 0.929596028; 36 | SLE GWAS | 2 | Atrial
fibrillation || id: ebi-a-GCST006414 | Inverse variance
weighted | 2 | 0.165274605 | 0.485600632 | 0.733592301; 37 | SLE GWAS | 9 | Atrial
fibrillation || id: ebi-a-GCST006414 | Inverse variance weighted | 7
| −0.037859974 | 0.047277407 | 0.42324466; 38 | SLE GWAS | 10 | Atrial fibrillation || id: ebi-
a-GCST006414 | Inverse variance weighted | 10
| −0.008740963 | 0.048470656 | 0.856889459; 39 | SLE GWAS | 7 | Atrial fibrillation || id: ebi-
a-GCST006414 | Inverse variance weighted | 7 | −0.063528515 | 0.039634287 | 1.09E−01;
40 | SLE GWAS | 37 | Atrial fibrillation || id: ebi-a-GCST006414 | Inverse variance
weighted | 33 | −0.020022652 | 0.006813891 | 3.30E−03; 41 | SLE GWAS | 5 | Atrial
fibrillation || id: ebi-a-GCST006414 | Inverse variance weighted | 4
| −0.093941463 | 0.04960611 | 5.83E−02; 42 | SLE GWAS | 79 | Atrial fibrillation || id: ebi-a-
GCST006414 | Inverse variance weighted | 65 | −0.100990021 | 0.010630487 | 2.09815E−21;
43 | SLE GWAS | 8 | Atrial fibrillation || id: ebi-a-GCST006414 | Inverse variance
weighted | 8 | 0.015829141 | 0.02566958 | 0.537465756; 44 | SLE GWAS | 21 | Atrial
fibrillation || id: ebi-a-GCST006414 | Inverse variance
weighted | 17 | 0.002435995 | 0.014041283 | 0.862267823; 45 | SLE GWAS | 43 | Atrial
fibrillation || id: ebi-a-GCST006414 | Inverse variance weighted | 40
| −0.008572032 | 0.006338247 | 0.176237937; 46 | SLE GWAS | 73 | Atrial fibrillation ||
id: ebi-a-GCST006414 | Inverse variance weighted | 63
| −0.020547326 | 0.006163634 | 8.57E−04; 1 | SLE GWAS | 130 | Cardiomyopathy || id: finn-
b-I9_CARDMYO | Inverse variance
weighted | 113 | 0.002469004 | 0.019944749 | 0.901479837; 2 | SLE
GWAS | 111 | Cardiomyopathy || id: finn-b-I9_CARDMYO | Inverse variance
weighted | 100 −0.039825071 | 0.015046882 | 0.008127361; 3 | SLE
GWAS | 175 | Cardiomyopathy || id: finn-b-I9_CARDMYO | Inverse variance
weighted | 146 | −0.04733559 | 0.0108498 | 1.28404E−05; 4 | SLE
GWAS | 30 | Cardiomyopathy || id: finn-b-I9_CARDMYO | Inverse variance
weighted | 28 | −0.074791496 | 0.024165271 | 0.001968134; 5 | SLE
GWAS | 383 | Cardiomyopathy || id: finn-b-I9_CARDMYO | Inverse variance
weighted | 331 | −0.076556786 | 0.011139609 | 6.30934E−12; 6 | SLE
GWAS | 5 | Cardiomyopathy || id: finn-b-I9_CARDMYO | Inverse variance weighted | 4
| −0.255863771 | 0.165000979 | 0.120978313; 7 | SLE GWAS | 39 | Cardiomyopathy ||
id: finn-b-I9_CARDMYO | Inverse variance weighted | 33
| −0.022530873 | 0.06820899 | 0.741157306; 8 | SLE GWAS | 67 | Cardiomyopathy || id: finn-
b-I9_CARDMYO | Inverse variance
weighted | 56 | 0.061905084 | 0.034916527 | 0.076237815; 9 | SLE
GWAS | 23 | Cardiomyopathy || id: finn-b-I9_CARDMYO | Inverse variance
weighted | 18 | −0.036040261 | 0.057071546 | 0.52771846; 10 | SLE
GWAS | 99 | Cardiomyopathy || id: finn-b-I9_CARDMYO | Inverse variance
weighted | 89 | 0.023442519 | 0.026805042 | 0.381815364; | 11 | SLE
GWAS | 183 | Cardiomyopathy || id: finn-b-I9_CARDMYO | Inverse variance
weighted | 156 | −0.159384843 | 0.015210238 | 1.08121E−25; 12 | SLE
GWAS | 199 | Cardiomyopathy || id: finn-b-I9_CARDMYO | Inverse variance
weighted | 165 | −0.07215765 | 0.015068108 | 1.6781E−06; 13 | SLE
GWAS | 55 | Cardiomyopathy || id: finn-b-I9_CARDMYO | Inverse variance
weighted | 49 | −0.091198683 | 0.024017143 | 0.00014632; 14 | SLE
GWAS | 173 | Cardiomyopathy || id: finn-b-I9_CARDMYO | Inverse variance
weighted | 155 | −0.006023344 | 0.009982103 | 0.546233103; 15 | SLE
GWAS | 3 | Cardiomyopathy || id: finn-b-I9_CARDMYO | Inverse variance weighted | 2
| −0.37350519 | 0.345813645 | 0.280108117; 16 | SLE GWAS | 53 | Cardiomyopathy ||
id: finn-b-I9_CARDMYO | Inverse variance weighted | 45
| −0.079525495 | 0.025429806 | 0.001764467; 17 | SLE GWAS | 40 | Cardiomyopathy ||
id: finn-b-I9_CARDMYO | Inverse variance weighted | 31
| −0.070624434 | 0.085163542 | 0.406945911; 18 | SLE GWAS | 196 | Cardiomyopathy ||
id: finn-b-I9_CARDMYO | Inverse variance
weighted | 173 | 0.002346612 | 0.016045314 | 0.883724768; 19 | SLE
GWAS | 24 | Cardiomyopathy || id: finn-b-I9_CARDMYO | Inverse variance
weighted | 19 | −0.39283833 | 0.044366746 | 8.41791E−19; 20 | SLE
GWAS | 2 | Cardiomyopathy || id: finn-b-I9_CARDMYO | Inverse variance weighted | 2
| −0.009948161 | 0.598474523 | 0.98673775; 21 | SLE GWAS | 222 | Cardiomyopathy ||
id: finn-b-I9_CARDMYO | Inverse variance weighted | 196
| −0.011740338 | 0.009479296 | 0.215521705; 22 | SLE GWAS | 48 | Cardiomyopathy ||
id: finn-b-I9_CARDMYO | Inverse variance
weighted | 42 | 0.088184699 | 0.029365116 | 0.002672949; 23 | SLE
GWAS | 12 | Cardiomyopathy || id: finn-b-I9_CARDMYO | Inverse variance
weighted | 12 | −0.009358897 | 0.061063458 | 0.878189224; 24 | SLE
GWAS | 17 | Cardiomyopathy || id: finn-b-I9_CARDMYO | Inverse variance
weighted | 14 | 0.080146139 | 0.127199227 | 0.52863993; 25 | SLE
GWAS | 10 | Cardiomyopathy || id: finn-b-I9_CARDMYO | Inverse variance
weighted | 7 | 0.499356473 | 0.260306956 | 0.05506829; 26 | SLE
GWAS | 38 | Cardiomyopathy || id: finn-b-I9_CARDMYO | Inverse variance
weighted | 30 | −0.042302877 | 0.054685547 | 0.43918752; 27 | SLE
GWAS | 9 | Cardiomyopathy || id: finn-b-I9_CARDMYO | Inverse variance
weighted | 7 | 0.071740353 | 0.132423515 | 0.587991189; 28 | SLE
GWAS | 11 | Cardiomyopathy || id: finn-b-I9_CARDMYO | Inverse variance
weighted | 9 | 0.091301962 | 0.136568571 | 0.503787041; 29 | SLE
GWAS | 15 | Cardiomyopathy || id: finn-b-I9_CARDMYO | Inverse variance
weighted | 13 | 0.044052748 | 0.084577793 | 0.602467933; 30 | SLE
GWAS | 10 | Cardiomyopathy || id: finn-b-I9_CARDMYO | Inverse variance
weighted | 6 | 0.021328488 | 0.118928526 | 0.857671757; 31 | SLE
GWAS | 14 | Cardiomyopathy || id: finn-b-I9_CARDMYO | Inverse variance
weighted | 11 | −0.116284603 | 0.122910278 | 0.344100949; 32 | SLE
GWAS | 5 | Cardiomyopathy || id: finn-b-I9_CARDMYO | Inverse variance
weighted | 4 | 0.82106674 | 0.545374219 | 0.13219282; 33 | SLE
GWAS | 51 | Cardiomyopathy || id: finn-b-I9_CARDMYO | Inverse variance
weighted | 45 | −0.201542555 | 0.039423571 | 3.1837E−07; 34 | SLE
GWAS | 31 | Cardiomyopathy || id: finn-b-I9_CARDMYO | Inverse variance
weighted | 25 | −0.319310608 | 0.041407417 | 1.24411E−14; 35 | SLE
GWAS | 19 | Cardiomyopathy || id: finn-b-I9_CARDMYO | Inverse variance
weighted | 16 | 0.040874158 | 0.052316303 | 0.434632466; 36 | SLE
GWAS | 2 | Cardiomyopathy || id: finn-b-I9_CARDMYO | Inverse variance
weighted | 2 | 1.02948374 | 2.032491961 | 0.612496518; 37 | SLE
GWAS | 9 | Cardiomyopathy || id: finn-b-I9_CARDMYO | Inverse variance weighted | 7
| −0.288320285 | 0.164246317 | 0.07918852; 38 | SLE GWAS | 10 | Cardiomyopathy ||
id: finn-b-I9_CARDMYO | Inverse variance
weighted | 10 | 0.006337812 | 0.125291495 | 0.959656589; 39 | SLE
GWAS | 7 | Cardiomyopathy || id: finn-b-I9_CARDMYO | Inverse variance
weighted | 7 | 0.085809801 | 0.156246592 | 0.582871836; 40 | SLE
GWAS | 37 | Cardiomyopathy || id: finn-b-I9_CARDMYO | Inverse variance
weighted | 33 | 0.063603126 | 0.030024628 | 0.034144155; 41 | SLE
GWAS | 5 | Cardiomyopathy || id: finn-b-I9_CARDMYO | Inverse variance weighted | 4
| −0.091742242 | 0.201213506 | 0.648429706; 42 | SLE GWAS | 79 | Cardiomyopathy ||
id: finn-b-I9_CARDMYO | Inverse variance
weighted | 65 | 0.151692219 | 0.047299014 | 0.001340848; 43 | SLE
GWAS | 8 | Cardiomyopathy || id: finn-b-I9_CARDMYO | Inverse variance weighted | 8
| −0.297487431 | 0.115516651 | 0.01001597; 44 | SLE GWAS | 21 | Cardiomyopathy ||
id: finn-b-I9_CARDMYO | Inverse variance
weighted | 17 | 0.061184764 | 0.056984526 | 0.282953398; 45 | SLE
GWAS | 43 | Cardiomyopathy || id: finn-b-I9_CARDMYO | Inverse variance
weighted | 40 | 0.061745937 | 0.027260453 | 0.023510404; 46 | SLE
GWAS | 73 | Cardiomyopathy || id: finn-b-I9_CARDMYO | Inverse variance
weighted | 63 | 0.029717828 | 0.026220281 | 0.257050124; 1 | SLE GWAS | 130 | Coronary
artery disease || id: ebi-a-GCST005194 | Inverse variance weighted | 112
| −0.039402541 | 0.006130859 | 1.30215E−10; 2 | SLE GWAS | 111 | Coronary artery disease
|| id: ebi-a-GCST005194 | Inverse variance
weighted | 100 | 0.013764509 | 0.004006626 | 5.92E−04; 3 | SLE GWAS | 175 | Coronary
artery disease || id: ebi-a-GCST005194 | Inverse variance
weighted | 147 | 0.020596293 | 0.002793648 | 1.67403E−13; 4 | SLE GWAS | 30 | Coronary
artery disease || id: ebi-a-GCST005194 | Inverse variance
weighted | 28 | 0.027210874 | 0.005066274 | 7.83084E−08; 5 | SLE GWAS | 383 | Coronary
artery disease || id: ebi-a-GCST005194 | Inverse variance
weighted | 332 | 0.007093381 | 0.003290484 | 0.031105106; 6 | SLE GWAS | 5 | Coronary
artery disease || id: ebi-a-GCST005194 | Inverse variance
weighted | 4 | 0.081469907 | 0.039192013 | 0.037641485; 7 | SLE GWAS | 39 | Coronary
artery disease || id: ebi-a-GCST005194 | Inverse variance
weighted | 33 | 0.024458475 | 0.018815389 | 0.193628857; 8 | SLE GWAS | 67 | Coronary
artery disease || id: ebi-a-GCST005194 | Inverse variance
weighted | 57 | 0.016028351 | 0.013517121 | 0.235708611; 9 | SLE GWAS | 23 | Coronary
artery disease || id: ebi-a-GCST005194 | Inverse variance
weighted | 18 | 0.039572721 | 0.024961382 | 0.112885071; 10 | SLE GWAS | 99 | Coronary
artery disease || id: ebi-a-GCST005194 | Inverse variance
weighted | 88 | 0.00155512 | 0.009497176 | 0.869931477; 11 | SLE GWAS | 183 | Coronary
artery disease || id: ebi-a-GCST005194 | Inverse variance
weighted | 153 | 0.00406774 | 0.005293402 | 0.442217073; 12 | SLE
GWAS | 199 | Coronary artery disease || id: ebi-a-GCST005194 | Inverse variance
weighted | 165 | 0.017599506 | 0.004469683 | 8.2325E−05; 13 | SLE GWAS | 55 | Coronary
artery disease || id: ebi-a-GCST005194 | Inverse variance
weighted | 49 | 0.029111718 | 0.008418114 | 0.000543751; 14 | SLE
GWAS | 173 | Coronary artery disease || id: ebi-a-GCST005194 | Inverse variance
weighted | 155 | −0.011990738 | 0.003368334 | 0.000371078; 15 | SLE
GWAS | 3 | Coronary artery disease || id: ebi-a-GCST005194 | Inverse variance
weighted | 2 | −0.079675638 | 0.100808459 | 0.429313708; 16 | SLE GWAS | 53 | Coronary
artery disease || id: ebi-a-GCST005194 | Inverse variance
weighted | 45 | 0.026534506 | 0.005254468 | 4.42056E−07; 17 | SLE GWAS | 40 | Coronary
artery disease || id: ebi-a-GCST005194 | Inverse variance
weighted | 31 | 0.061936519 | 0.029223934 | 0.034058664; 18 SLE
GWAS | 196 | Coronary artery disease || id: ebi-a-GCST005194 | Inverse variance
weighted | 173 | 0.020833422 | 0.004934227 | 2.41901E−05; 19 | SLE
GWAS | 24 | Coronary artery disease | id: ebi-a-GCST005194 | Inverse variance
weighted | 19 | −0.126245868 | 0.010036026 | 2.74559E−36; 20 | SLE GWAS | 2 | Coronary
artery disease || id: ebi-a-GCST005194 | Inverse variance
weighted | 2 | 0.275873213 | 0.320243194 | 0.388990741; 21 | SLE GWAS | 222 | Coronary
artery disease || id: ebi-a-GCST005194 | Inverse variance | 196
| −0.00853801 | 0.003661643 | 0.019714205; 22 | SLE GWAS | 48 | Coronary artery disease ||
id: ebi-a-GCST005194 | Inverse variance | 42
| −0.056140228 | 0.008482171 | 3.62579E−11; 23 | SLE GWAS | 12 | Coronary artery disease
|| id: ebi-a-GCST005194 | Inverse variance
weighted | 12 | 0.007308402 | 0.015748499 | 0.642597775; 24 | SLE GWAS | 17 | Coronary
artery disease || id: ebi-a-GCST005194 | Inverse variance weighted 14
| −0.005128747 | 0.038264899 | 0.893376727; 25 | SLE GWAS | 10 | Coronary artery disease
|| id: ebi-a-GCST005194 | Inverse variance weighted | 7
| −0.041843218 | 0.113752499 | 0.712989269; 26 | SLE GWAS | 38 | Coronary artery disease
|| id: ebi-a-GCST005194 | Inverse variance
weighted | 30 | 0.025794359 | 0.015992023 | 0.106755025; 27 | SLE GWAS | 9 | Coronary
artery disease || id: ebi-a-GCST005194 | Inverse variance
weighted | 7 | 0.094322108 | 0.067436085 | 0.161906277; 28 | SLE GWAS | 11 | Coronary
artery disease || id: ebi-a-GCST005194 | Inverse variance
weighted | 8 | 0.080547858 | 0.060651697 | 0.184165017; 29 | SLE GWAS | 15 | Coronary
artery disease || id: ebi-a-GCST005194 | Inverse variance
weighted | 13 | 0.026480369 | 0.033376593 | 0.427555621; 30 | SLE GWAS | 10 | Coronary
artery disease || id: ebi-a-GCST005194 | Inverse variance
weighted | 6 | 0.012375875 | 0.025902811 | 0.63280598; 31 | SLE GWAS | 14 | Coronary
artery disease || id: ebi-a-GCST005194 | Inverse variance
weighted | 11 | 0.083799516 | 0.03222313 | 0.009306052; 32 | SLE GWAS | 5 | Coronary
artery disease || id: ebi-a-GCST005194 | Inverse variance
weighted | 4 | 0.172274435 | 0.083197597 | 0.038390019; 33 | SLE GWAS | 51 | Coronary
artery disease || id: ebi-a-GCST005194 | Inverse variance weighted | 45
| −0.01061875 | 0.01830562 | 0.561859704; 34 | SLE GWAS | 31 | Coronary artery disease ||
id: ebi-a-GCST005194 | Inverse variance weighted | 25
| −0.094832056 | 0.015361258 | 6.6813E−10; 35 | SLE GWAS | 19 | Coronary artery disease ||
id: ebi-a-GCST005194 | Inverse variance
weighted | 16 | 0.069901897 | 0.01324241 | 1.30147E−07; 36 | SLE GWAS | 2 | Coronary
artery disease || id: ebi-a-GCST005194 | Inverse variance weighted | 2
| −0.229730844 | 0.239799972 | 0.338057562; 37 | SLE GWAS | 9 | Coronary artery disease ||
id: ebi-a-GCST005194 | Inverse variance
weighted | 7 | 0.025379979 | 0.074684037 | 0.733983514; 38 | SLE GWAS | 10 | Coronary
artery disease || id: ebi-a-GCST005194 | Inverse variance weighted | 10
| −0.001991513 | 0.048284789 | 0.967100464; 39 | SLE GWAS | 7 | Coronary artery disease ||
id: ebi-a-GCST005194 | Inverse variance weighted | 7
| −0.020541443 | 0.030679014 | 0.503138274; 40 | SLE GWAS | 37 | Coronary artery disease
|| id: ebi-a-GCST005194 | Inverse variance weighted | 33
| −0.063284571 | 0.005836843 | 2.17043E−27; 41 | SLE GWAS | 5 | Coronary artery disease ||
id: ebi-a-GCST005194 | Inverse variance
weighted | 4 | 0.122985753 | 0.047303564 | 0.009324394; 42 | SLE GWAS | 79 | Coronary
artery disease || id: ebi-a-GCST005194 | Inverse variance
weighted | 65 | 0.0212703 | 0.009582954 | 0.026446106; 43 | SLE GWAS | 8 | Coronary
artery disease || id: ebi-a-GCST005194 | Inverse variance weighted | 8 | 7.18604E−05
| 0.024489339 | 0.997658728; 44 | SLE GWAS | 21 | Coronary artery disease || id: ebi-a-
GCST005194 | Inverse variance weighted | 17 | 0.044359369 | 0.011298657 | 8.63435E−05;
45 | SLE GWAS | 43 | Coronary artery disease || id: ebi-a-GCST005194 | Inverse variance
weighted | 40 | −0.034328045 | 0.012056477 | 0.004409594; 46 | SLE
GWAS | 73 | Coronary artery disease || id: ebi-a-GCST005194 | Inverse variance
weighted | 63 | −0.043765551 | 0.008894917 | 8.6417E−07; 1 | SLE GWAS | 130 | Coronary
artery disease || id: ebi-a-GCST005195 | Inverse variance weighted | 112
| −0.035670403 | 0.006639751 | 7.77603E−08; 2 | SLE GWAS | 111 | Coronary artery disease
|| id: ebi-a-GCST005195 | Inverse variance
weighted | 100 | 0.012399183 | 0.004721629 | 0.008638483; 3 | SLE
GWAS | 175 | Coronary artery disease || id: ebi-a-GCST005195 | Inverse variance
weighted | 147 | 0.021295836 | 0.003054883 | 3.14515E−12; 4 SLE GWAS | 30 | Coronary
artery disease || id: ebi-a-GCST005195 | Inverse variance
weighted | 28 | 0.026742668 | 0.006201783 | 1.61716E−05; 5 | SLE GWAS | 383 | Coronary
artery disease || id: ebi-a-GCST005195 | Inverse variance
weighted | 332 | 0.00445621 | 0.003318125 | 0.179274982; 6 | SLE GWAS | 5 | Coronary
artery disease || id: ebi-a-GCST005195 | Inverse variance
weighted | 4 | 0.127306876 | 0.049736171 | 0.010477953; 7 | SLE GWAS | 39 | Coronary
artery disease || id: ebi-a-GCST005195 | Inverse variance
weighted | 33 | 0.013223729 | 0.017516095 | 0.450280591; 8 | SLE GWAS | 67 | Coronary
artery disease || id: ebi-a-GCST005195 | Inverse variance
weighted | 57 | 0.003262667 | 0.011908384 | 0.784099468; 9 | SLE GWAS | 23 | Coronary
artery disease || id: ebi-a-GCST005195 | Inverse variance
weighted | 18 | 0.053479287 | 0.02858538 | 0.061364277; 10 | SLE GWAS | 99 | Coronary
artery disease || id: ebi-a-GCST005195 | Inverse variance
weighted | 88 | 0.001830541 | 0.009964083 | 0.854237862; 11 | SLE
GWAS | 183 | Coronary artery disease || id: ebi-a-GCST005195 | Inverse variance
weighted | 153 | 0.003069816 | 0.005773071 | 0.594900919; 12 | SLE
GWAS | 199 | Coronary artery disease || id: ebi-a-GCST005195 | Inverse variance
weighted | 165 | 0.001522044 | 0.005106208 | 0.765644267; 13 | SLE
GWAS | 55 | Coronary artery disease || id: ebi-a-GCST005195 | Inverse variance
weighted | 49 | 0.027254731 | 0.007917645 | 0.000576839; 14 | SLE
GWAS | 173 | Coronary artery disease || id: ebi-a-GCST005195 | Inverse variance
weighted | 155 | −0.010587734 | 0.003629041 | 0.00352847; 15 | SLE GWAS | 3 | Coronary
artery disease || id: ebi-a-GCST005195 | Inverse variance weighted | 2
| −0.023744567 | 0.088314595 | 0.788034766; 16 | SLE GWAS | 53 | Coronary artery disease
|| id: ebi-a-GCST005195 | Inverse variance
weighted | 45 | 0.024335788 | 0.00650617 | 0.000183715; 17 | SLE GWAS | 40 | Coronary
artery disease || id: ebi-a-GCST005195 | Inverse variance
weighted | 31 | 0.054054825 | 0.032034752 | 0.091530158; 18 | SLE
GWAS | 196 | Coronary artery disease || id: ebi-a-GCST005195 | Inverse variance
weighted | 174 | 0.016796113 | 0.00498862 | 0.000760222; 19 | SLE GWAS | 24 | Coronary
artery disease || id: ebi-a-GCST005195 | Inverse variance weighted | 19
| −0.170467531 | 0.012380785 | 3.93131E−43; 20 | SLE GWAS | 2 | Coronary artery disease ||
id: ebi-a-GCST005195 | Inverse variance
weighted | 2 | 0.321811117 | 0.341939117 | 0.346635313; 21 | SLE GWAS | 222 | Coronary
artery disease || id: ebi-a-GCST005195 | Inverse variance weighted | 196
| −0.007632097 | 0.003931663 | 0.052235505; 22 | SLE GWAS | 48 | Coronary artery disease
|| id: ebi-a-GCST005195 | Inverse variance weighted | 42
| −0.048759623 | 0.007636281 | 1.71109E−10; 23 | SLE GWAS | 12 | Coronary artery disease
|| id: ebi-a-GCST005195 | Inverse variance
weighted | 12 | 0.002966845 | 0.016577873 | 0.85796585; 24 | SLE GWAS | 17 | Coronary
artery disease || id: ebi-a-GCST005195 | Inverse variance weighted | 14
| −0.031508047 | 0.042998723 | 0.463700536; 25 | SLE GWAS | 10 | Coronary artery disease
|| id: ebi-a-GCST005195 | Inverse variance weighted | 7
| −0.086847663 | 0.078444916 | 0.268243551; 26 | SLE GWAS | 38 | Coronary artery disease
|| id: ebi-a-GCST005195 | Inverse variance
weighted | 30 | 0.012685968 | 0.014136076 | 0.369495945; 27 | SLE GWAS | 9 | Coronary
artery disease || id: ebi-a-GCST005195 | Inverse variance
weighted | 7 | 0.091731332 | 0.067651505 | 0.175118497; 28 | SLE GWAS | 11 | Coronary
artery disease || id: ebi-a-GCST005195 | Inverse variance
weighted | 8 | 0.080946308 | 0.062699017 | 0.196693292; 29 | SLE GWAS | 15 | Coronary
artery disease || id: ebi-a-GCST005195 | Inverse variance
weighted | 13 | 0.014190104 | 0.035075496 | 0.685801772; 30 | SLE GWAS | 10 | Coronary
artery disease || id: ebi-a-GCST005195 | Inverse variance
weighted | 6 | 0.003243829 | 0.031015406 | 0.916703011; 31 | SLE GWAS | 14 | Coronary
artery disease || id: ebi-a-GCST005195 | Inverse variance
weighted | 11 | 0.036292668 | 0.043330953 | 0.402272743; 32 | SLE GWAS | 5 | Coronary
artery disease || id: ebi-a-GCST005195 | Inverse variance
weighted | 4 | 0.139057206 | 0.107799115 | 0.197062424; 33 | SLE GWAS | 51 | Coronary
artery disease || id: ebi-a-GCST005195 | Inverse variance weighted | 45
| −0.001944015 | 0.020914229 | 0.925941854; 34 | SLE GWAS | 31 | Coronary artery disease
|| id: ebi-a-GCST005195 | Inverse variance weighted | 25
| −0.134074657 | 0.017507736 | 1.88816E−14; 35 SLE GWAS | 19 | Coronary artery disease
|| id: ebi-a-GCST005195 | Inverse variance
weighted | 16 | 0.049920863 | 0.01622648 | 0.002094421; 36 | SLE GWAS | 2 | Coronary
artery disease || id: ebi-a-GCST005195 | Inverse variance weighted | 2
| −0.39982173 | 0.290231516 | 0.168328105; 37 | SLE GWAS | 9 | Coronary artery disease ||
id: ebi-a-GCST005195 | Inverse variance
weighted | 7 | 0.0610824 | 0.081543437 | 0.453810155; 38 | SLE GWAS | 10 | Coronary
artery disease || id: ebi-a-GCST005195 | Inverse variance
weighted | 10 | 0.000834425 | 0.062289555 | 0.98931193; 39 | SLE GWAS | 7 | Coronary
artery disease || id: ebi-a-GCST005195 | Inverse variance weighted | 7
| −0.035161816 | 0.032858244 | 0.284571446; 40 | SLE GWAS | 37 | Coronary artery disease
|| id: ebi-a-GCST005195 | Inverse variance weighted | 33
| −0.053076837 | 0.006996621 | 3.2976E−14; 41 | SLE GWAS | 5 | Coronary artery disease ||
id: ebi-a-GCST005195 | Inverse variance
weighted | 4 | 0.164838434 | 0.067317762 | 0.0143388; 42 | SLE GWAS | 79 | Coronary
artery disease || id: ebi-a-GCST005195 | Inverse variance
weighted | 65 | 0.016510813 | 0.010304001 | 0.109073998; 43 | SLE GWAS | 8 | Coronary
artery disease || id: ebi-a-GCST005195 | Inverse variance weighted | 8
| −0.020984642 | 0.030850807 | 0.496379419; 44 | SLE GWAS | 21 | Coronary artery disease
|| id: ebi-a-GCST005195 | Inverse variance
weighted | 17 | 0.001411325 | 0.014292605 | 0.921340639; 45 | SLE GWAS | 43 | Coronary
artery disease || id: ebi-a-GCST005195 | Inverse variance weighted | 40
| −0.028039282 | 0.01154886 | 0.015187237; 46 | SLE GWAS | 73 | Coronary artery disease ||
id: ebi-a-GCST005195 | Inverse variance weighted | 63
| −0.03651529 | 0.007958642 | 4.47233E−06; 1 | SLE GWAS | 130 | Ischemic stroke || id: ieu-
a-1108 | Inverse variance weighted | 77 | 0.01984001 | 0.016514605 | 0.229611005; 2 | SLE
GWAS | 111 | Ischemic stroke || id: ieu-a-1108 | Inverse variance
weighted | 83 | 0.021740098 | 0.01019662 | 0.032999564; 3 | SLE GWAS | 175 | Ischemic
stroke || id: ieu-a-1108 | Inverse variance
weighted | 126 | 0.002327564 | 0.007263225 | 0.748620571; 4 | SLE GWAS | 30 | Ischemic
stroke || id: ieu-a-1108 | Inverse variance
weighted | 23 | 0.060550165 | 0.015966022 | 0.000149175; 5 | SLE GWAS | 383 | Ischemic
stroke || id: ieu-a-1108 | Inverse variance
weighted | 278 | 0.044477771 | 0.006706738 | 3.31609E−11; 6 | SLE GWAS | 5 | Ischemic
stroke || id: ieu-a-1108 | Inverse variance
weighted | 3 | 0.029008466 | 0.11238495 | 0.796316613; 7 | SLE GWAS | 39 | Ischemic
stroke || id: ieu-a-1108 | Inverse variance weighted | 28
| −0.059160063 | 0.044368079 | 1.82E−01; 8 | SLE GWAS | 67 | Ischemic stroke || id: ieu-a-
1108 | Inverse variance weighted | 45 | 0.009630276 | 0.022671316 | 0.67099851; 9 | SLE
GWAS | 23 | Ischemic stroke || id: ieu-a-1108 | Inverse variance weighted | 14
| −0.002248969 | 0.037128437 | 0.951699531; 10 | SLE GWAS | 99 | Ischemic stroke || id: ieu-
a-1108 | Inverse variance weighted | 74 | 0.042327648 | 0.022726262 | 0.062532742;
11 | SLE GWAS | 183 | Ischemic stroke || id: ieu-a-1108 | Inverse variance
weighted | 134 | 0.043018358 | 0.011801394 | 0.000267191; 12 | SLE
GWAS | 199 | Ischemic stroke || id: ieu-a-1108 | Inverse variance
weighted | 144 | 0.070167993 | 0.008960832 | 4.85849E−15; 13 | SLE
GWAS | 55 | Ischemic stroke || id: ieu-a-1108 Inverse variance
weighted | 44 | 0.068990446 | 0.015851935 | 1.34792E−05; 14 | SLE
GWAS | 173 | Ischemic stroke || id: ieu-a-1108 | Inverse variance
weighted | 110 | 0.03557081 | 0.007490747 | 2.04796E−06; 16 | SLE GWAS | 53 | Ischemic
stroke || id: ieu-a-1108 | Inverse variance
weighted | 38 | 0.057133268 | 0.016683729 | 0.000615954; 17 | SLE GWAS | 40 | Ischemic
stroke || id: ieu-a-1108 | Inverse variance
weighted | 26 | 0.091590028 | 0.05507984 | 0.096340591; 18 | SLE GWAS | 196 | Ischemic
stroke || id: ieu-a-1108 | Inverse variance
weighted | 140 | 0.042447138 | 0.010373242 | 4.27699E−05; 19 | SLE
GWAS | 24 | Ischemic stroke || id: ieu-a-1108 | Inverse variance
weighted | 18 | 0.150798856 | 0.026860397 | 1.97507E−08; 21 | SLE
GWAS | 222 | Ischemic stroke || id: ieu-a-1108 | Inverse variance
weighted | 152 | 0.033692022 | 0.007240964 | 3.2718E−06; 22 | SLE GWAS | 48 | Ischemic
stroke || id: ieu-a-1108 | Inverse variance weighted | 22
| −0.103896337 | 0.02583139 | 5.76825E−05; 23 | SLE GWAS | 12 | Ischemic stroke || id: ieu-
a-1108 | Inverse variance weighted | 11 | 0.02795623 | 0.035802718 | 0.43489602; 24 | SLE
GWAS | 17 | Ischemic stroke || id: ieu-a-1108 | Inverse variance weighted | 12
| −0.023039079 | 0.068641427 | 0.737139385; 25 | SLE GWAS | 10 | Ischemic stroke || id: ieu-
a-1108 | Inverse variance weighted | 6 | 0.689112216 | 0.322312907 | 0.032514925;
26 | SLE GWAS | 38 | Ischemic stroke || id: ieu-a-1108 | Inverse variance
weighted | 25 | 0.00214204 | 0.033130257 | 0.94844862; 27 | SLE GWAS | 9 | Ischemic
stroke || id: ieu-a-1108 | Inverse variance
weighted | 6 | 0.052906872 | 0.078613429 | 0.500947013; 28 | SLE GWAS | 11 | Ischemic
stroke || id: ieu-a-1108 | Inverse variance
weighted | 8 | 0.102106584 | 0.078816079 | 0.195146251; 29 | SLE GWAS | 15 | Ischemic
stroke || id: ieu-a-1108 | Inverse variance weighted | 11
| −0.060901508 | 0.064573558 | 0.34561244; 30 | SLE GWAS | 10 | Ischemic stroke || id: ieu-
a-1108 | Inverse variance weighted | 5 | −0.231552049 | 0.120369385 | 0.054394842;
31 | SLE GWAS | 14 | Ischemic stroke || id: ieu-a-1108 | Inverse variance
weighted | 11 | 0.074536576 | 0.072261811 | 0.30231602; 32 | SLE GWAS | 5 | Ischemic
stroke || id: ieu-a-1108 | Inverse variance
weighted | 4 | 0.227252233 | 0.288561615 | 0.430969027; 33 | SLE GWAS | 51 | Ischemic
stroke || id: ieu-a-1108 | Inverse variance
weighted | 37 | 0.07895245 | 0.023886367 | 0.000948629; 34 | SLE GWAS | 31 | Ischemic
stroke || id: ieu-a-1108 | Inverse variance
weighted | 21 | 0.151586973 | 0.025164826 | 1.70407E−09; 35 | SLE GWAS | 19 | Ischemic
stroke || id: ieu-a-1108 | Inverse variance
weighted | 13 | 0.017366942 | 0.058084229 | 0.764943237; 36 | SLE GWAS | 2 | Ischemic
stroke || id: ieu-a-1108 | Inverse variance weighted | 2
| −0.424924569 | 1.902436713 | 0.823256845; 37 | SLE GWAS | 9 | Ischemic stroke || id: ieu-
a-1108 | Inverse variance weighted | 6 | −0.026255423 | 0.09336405 | 0.778545075;
38 | SLE GWAS | 10 | Ischemic stroke || id: ieu-a-1108 | Inverse variance weighted | 10
| −0.2691856 | 0.08223735 | 0.001063084; 39 | SLE GWAS | 7 | Ischemic stroke || id: ieu-a-
1108 | Inverse variance weighted | 7 | 0.016609238 | 0.075306777 | 0.825439437; 40 | SLE
GWAS | 37 | Ischemic stroke || id: ieu-a-1108 | Inverse variance weighted | 12
| −0.117971489 | 0.034327009 | 0.000588857; 41 | SLE GWAS | 5 | Ischemic stroke || id: ieu-
a-1108 | Inverse variance weighted | 4 | 0.06218935 | 0.12413726 | 0.616390494; 42 | SLE
GWAS | 79 | Ischemic stroke || id: ieu-a-1108 | Inverse variance
weighted | 61 | 0.003859482 | 0.018640422 | 0.835971542; 43 | SLE GWAS | 8 | Ischemic
stroke || id: ieu-a-1108 | Inverse variance weighted | 8
| −0.030644826 | 0.066886945 | 0.646838539; 44 | SLE GWAS | 21 | Ischemic stroke || id: ieu-
a-1108 | Inverse variance weighted | 17 | 0.052649183 | 0.033116069 | 0.111871342;
45 | SLE GWAS | 43 | Ischemic stroke || id: ieu-a-1108 | Inverse variance weighted | 19
| −0.041469616 | 0.028609118 | 0.147191166; 46 | SLE GWAS | 73 | Ischemic stroke || id: ieu-
a-1108 | Inverse variance weighted | 38 | −0.052297135 | 0.027575981 | 0.057897369;
1 | SLE GWAS | 130 | Myocardial infarction || id: ebi-a-GCST011365 | Inverse variance
weighted | 112 | −0.042043607 | 0.007390143 | 1.27676E−08; 2 | SLE
GWAS | 111 | Myocardial infarction || id: ebi-a-GCST011365 Inverse variance
weighted | 100 | 0.028192479 | 0.005055496 | 2.45265E−08; 3 | SLE
GWAS | 175 | Myocardial infarction || id: ebi-a-GCST011365 | Inverse variance
weighted | 147 | 0.028748948 | 0.003334521 | 6.60171E−18; 4 | SLE
GWAS | 30 | Myocardial infarction || id: ebi-a-GCST011365 | Inverse variance
weighted | 28 | 0.054366219 | 0.006760324 | 8.84169E−16; 5 | SLE
GWAS | 383 | Myocardial infarction || id: ebi-a-GCST011365 | Inverse variance
weighted | 332 | 0.011680594 | 0.003073523 | 0.000144467; 6 | SLE
GWAS | 5 | Myocardial infarction || id: ebi-a-GCST011365 | Inverse variance
weighted | 4 | 0.036364496 | 0.051445447 | 0.479656285; 7 | SLE GWAS | 39 | Myocardial
infarction || id: ebi-a-GCST011365 | Inverse variance
weighted | 33 | 0.050230155 | 0.021891906 | 0.021763949; 8 | SLE
GWAS | 67 | Myocardial infarction || id: ebi-a-GCST011365 | Inverse variance
weighted | 57 | 0.00360266 | 0.015036669 | 0.810646829; 9 | SLE GWAS | 23 | Myocardial
infarction || id: ebi-a-GCST011365 | Inverse variance
weighted | 18 | 0.029066814 | 0.024962066 | 0.244245972; 10 | SLE
GWAS | 99 | Myocardial infarction || id: ebi-a-GCST011365 | Inverse variance
weighted | 88 | −0.0021469 | 0.011749816 | 0.85501946; 11 | SLE
GWAS | 183 | Myocardial infarction || id: ebi-a-GCST011365 | Inverse variance
weighted | 155 | 0.032011821 | 0.006185022 | 2.27057E−07; 12 | SLE
GWAS | 199 | Myocardial infarction || id: ebi-a-GCST011365 | Inverse variance
weighted | 165 | 0.02862855 | 0.006537597 | 1.19191E−05; 13 | SLE
GWAS | 55 | Myocardial infarction || id: ebi-a-GCST011365 | Inverse variance
weighted | 49 | 0.05407612 | 0.009333799 | 6.89013E−09; 14 | SLE
GWAS | 173 | Myocardial infarction || id: ebi-a-GCST011365 | Inverse variance
weighted | 155 −0.026017813 | 0.003441185 | 4.00867E−14; 15 | SLE
GWAS | 3 | Myocardial infarction || id: ebi-a-GCST011365 | Inverse variance
weighted | 2 | −0.087186337 | 0.097614959 | 0.371768418; 16 | SLE
GWAS | 53 | Myocardial infarction || id: ebi-a-GCST011365 | Inverse variance
weighted | 45 | 0.050937285 | 0.007042304 | 4.7229E−13; 17 | SLE
GWAS | 40 | Myocardial infarction || id: ebi-a-GCST011365 | Inverse variance
weighted | 31 | 0.098899062 | 0.033437993 | 0.003099583; 18 | SLE
GWAS | 196 | Myocardial infarction || id: ebi-a-GCST011365 | Inverse variance
weighted | 174 | 0.032595737 | 0.006156794 | 1.19492E−07; 19 | SLE
GWAS | 24 | Myocardial infarction || id: ebi-a-GCST011365 | Inverse variance
weighted | 19 | −0.183382607 | 0.013658112 | 4.22113E−41; 20 | SLE
GWAS | 2 | Myocardial infarction || id: ebi-a-GCST011365 | Inverse variance
weighted | 2 | 0.185966596 | 0.288634616 | 0.519382459; 21 | SLE
GWAS | 222 | Myocardial infarction || id: ebi-a-GCST011365 | Inverse variance
weighted | 196 | −0.020068248 | 0.004457856 | 6.73894E−06; 22 | SLE
GWAS | 48 | Myocardial infarction || id: ebi-a-GCST011365 | Inverse variance
weighted | 42 | −0.041877186 | 0.008431431 | 6.80686E−07; 23 | SLE
GWAS | 12 | Myocardial infarction || id: ebi-a-GCST011365 | Inverse variance
weighted | 12 | 0.016986103 | 0.016333686 | 0.298366357; 24 | SLE
GWAS | 17 | Myocardial infarction || id: ebi-a-GCST011365 | Inverse variance
weighted | 14 | −0.02109392 | 0.042263358 | 0.617704307; 25 | SLE
GWAS | 10 | Myocardial infarction || id: ebi-a-GCST011365 Inverse variance
weighted | 7 | 0.064725613 | 0.144002132 | 0.653087764; 26 | SLE
GWAS | 38 | Myocardial infarction || id: ebi-a-GCST011365 | Inverse variance
weighted | 30 | 0.029239524 | 0.021853581 | 0.1809049; 27 | SLE GWAS | 9 | Myocardial
infarction || id: ebi-a-GCST011365 | Inverse variance
weighted | 7 | 0.073783223 | 0.079911512 | 0.355844866; 28 | SLE
GWAS | 11 | Myocardial infarction || id: ebi-a-GCST011365 | Inverse variance
weighted | 8 | 0.050632449 | 0.073107888 | 0.48857842; 29 | SLE GWAS | 15 | Myocardial
infarction || id: ebi-a-GCST011365 | Inverse variance
weighted | 13 | 0.009535326 | 0.031547401 | 0.762458502; 30 | SLE
GWAS | 10 | Myocardial infarction || id: ebi-a-GCST011365 | Inverse variance
weighted | 6 | −0.001325089 | 0.035557002 | 0.970272429; 31 | SLE
GWAS | 14 | Myocardial infarction || id: ebi-a-GCST011365 | Inverse variance
weighted | 11 | 0.074502937 | 0.04407423 | 0.090952067; 32 SLE GWAS | 5 | Myocardial
infarction || id: ebi-a-GCST011365 | Inverse variance
weighted | 4 | 0.111200714 | 0.114238795 | 0.330351625; 33 SLE
GWAS | 51 | Myocardial infarction || id: ebi-a-GCST011365 | Inverse variance
weighted 45 | 0.00876014 | 0.018201352 | 0.630309964; 34 | SLE
GWAS | 31 | Myocardial infarction || id: ebi-a-GCST011365 | Inverse variance
weighted | 25 | −0.148193505 | 0.018045393 | 2.17059E−16; 35 | SLE
GWAS | 19 | Myocardial infarction || id: ebi-a-GCST011365 | Inverse variance
weighted | 16 | 0.058715515 | 0.014691323 | 6.42556E−05; 36 | SLE
GWAS | 2 | Myocardial infarction || id: ebi-a-GCST011365 | Inverse variance
weighted | 2 | −0.077759651 | 0.480569716 | 0.871457665; 37 | SLE
GWAS | 9 | Myocardial infarction || id: ebi-a-GCST011365 | Inverse variance
weighted | 7 | 0.035457468 | 0.066126036 | 0.591813154; 38 | SLE
GWAS | 10 | Myocardial infarction || id: ebi-a-GCST011365 | Inverse variance
weighted | 10 | −0.015915497 | 0.04103928 | 0.698155675; 39 | SLE
GWAS | 7 | Myocardial infarction || id: ebi-a-GCST011365 | Inverse variance
weighted | 7 | −0.034515184 | 0.03519235 | 0.326712012; 40 | SLE
GWAS | 37 | Myocardial infarction || id: ebi-a-GCST011365 | Inverse variance
weighted | 33 | −0.04986875 | 0.007924458 | 3.11353E−10; 41 | SLE
GWAS | 5 | Myocardial infarction || id: ebi-a-GCST011365 | Inverse variance
weighted | 4 | 0.052977241 | 0.056552255 | 0.348869735; 42 | SLE
GWAS | 79 | Myocardial infarction || id: ebi-a-GCST011365 | Inverse variance
weighted | 65 | 0.01277825 | 0.01046276 | 0.22196948; 43 | SLE GWAS | 8 | Myocardial
infarction || id: ebi-a-GCST011365 | Inverse variance
weighted | 8 | 0.013427559 | 0.027022144 | 0.619252901; 44 | SLE
GWAS | 21 | Myocardial infarction || id: ebi-a-GCST011365 | Inverse variance
weighted | 17 | 0.087992828 | 0.014766612 | 2.53935E−09; 45 | SLE
GWAS | 43 | Myocardial infarction || id: ebi-a-GCST011365 | Inverse variance
weighted | 40 | −0.017532499 | 0.014302136 | 0.220249212; 46 | SLE
GWAS | 73 | Myocardial infarction || id: ebi-a-GCST011365 | Inverse variance
weighted | 64 | −0.032737076 | 0.009137758 | 0.000340171; 1 | SLE
GWAS | 130 Myocardial infarction || id: ieu-a-798 | Inverse variance weighted | 113
| −0.039892016 | 0.007990072 | 5.95416E−07; 2 | SLE GWAS | 111 | Myocardial infarction ||
id: ieu-a-798 | Inverse variance weighted | 100 | 0.017669763 | 0.005710363 | 0.001972563;
3 | SLE GWAS | 175 | Myocardial infarction || id: ieu-a-798 | Inverse variance
weighted | 147 | 0.014371369 | 0.004252835 | 0.000726855; 4 | SLE
GWAS | 30 | Myocardial infarction || id: ieu-a-798 | Inverse variance
weighted | 28 | 0.038463148 | 0.009553637 | 5.67285E−05; 5 | SLE
GWAS | 383 | Myocardial infarction || id: ieu-a-798 | Inverse variance
weighted | 332 | 0.004070734 | 0.003985433 | 0.307063521; 6 | SLE
GWAS | 5 | Myocardial infarction || id: ieu-a-798 | Inverse variance weighted 4
| −0.002607206 | 0.069286805 | 0.969983335; 7 | SLE GWAS | 39 | Myocardial infarction ||
id: ieu-a-798 | Inverse variance weighted | 33 | 0.070307294 | 0.025639334 | 0.00610356;
8 | SLE GWAS | 67 | Myocardial infarction || id: ieu-a-798 | Inverse variance
weighted | 57 | 0.023111953 | 0.016090732 | 0.150902252; 9 | SLE
GWAS | 23 | Myocardial infarction || id: ieu-a-798 | Inverse variance
weighted | 18 | 0.026215933 | 0.02909997 | 0.367645692; 10 | SLE
GWAS | 99 | Myocardial infarction || id: ieu-a-798 | Inverse variance | 89
| −0.009886481 | 0.013286175 | 0.456805124; 11 | SLE GWAS | 183 | Myocardial infarction
|| id: ieu-a-798 Inverse variance
weighted | 155 | 0.019804338 | 0.006846198 | 0.003818856; 12 | SLE
GWAS | 199 | Myocardial infarction || id: ieu-a-798 | Inverse variance
weighted | 165 | 0.043693323 | 0.005573855 | 4.54229E−15; 13 | SLE
GWAS | 55 | Myocardial infarction || id: ieu-a-798 | Inverse variance
weighted | 49 | 0.044165082 | 0.01168331 | 0.000156711; 14 | SLE
GWAS | 173 | Myocardial infarction || id: ieu-a-798 | Inverse variance weighted | 155
| −0.021181035 | 0.004219922 | 5.18614E−07; 15 | SLE GWAS | 3 | Myocardial infarction ||
id: ieu-a-798 | Inverse variance weighted | 2 | −0.181451887 | 0.130275647 | 0.16367107;
16 | SLE GWAS | 53 | Myocardial infarction || id: ieu-a-798 | Inverse variance
weighted | 45 | 0.034518544 | 0.009882888 | 0.000478058; 17 | SLE
GWAS | 40 | Myocardial infarction || id: ieu-a-798 | Inverse variance
weighted | 31 | 0.056258401 | 0.046472152 | 0.226055231; 18 | SLE
GWAS | 196 | Myocardial infarction || id: ieu-a-798 | Inverse variance
weighted | 173 | 0.025325729 | 0.007258104 | 0.000484281; 19 | SLE
GWAS | 24 | Myocardial infarction || id: ieu-a-798 | Inverse variance weighted | 19
| −0.039371584 | 0.019460653 | 0.043058938; 20 | SLE GWAS | 2 | Myocardial infarction ||
id: ieu-a-798 | Inverse variance weighted | 2 | 0.04010741 | 0.262841424 | 0.878720299;
21 | SLE GWAS | 222 | Myocardial infarction || id: ieu-a-798 | Inverse variance
weighted | 195 | −0.017862027 | 0.004908269 | 0.000273518; 22 | SLE
GWAS | 48 | Myocardial infarction || id: ieu-a-798 | Inverse variance | 42
| −0.054843873 | 0.011417656 | 1.55973E−06; 23 | SLE GWAS | 12 | Myocardial infarction ||
id: ieu-a-798 | Inverse variance weighted | 12 | −0.003446681 | 0.021085749 | 0.870156096;
24 | SLE GWAS | 17 | Myocardial infarction || id: ieu-a-798 | Inverse variance
weighted | 14 | 0.018167695 | 0.047746787 | 0.703573633; 25 | SLE
GWAS | 10 | Myocardial infarction || id: ieu-a-798 | Inverse variance
weighted | 7 | 0.125742731 | 0.181821983 | 0.489207054; 26 | SLE
GWAS | 38 | Myocardial infarction || id: ieu-a-798 | Inverse variance
weighted | 30 | 0.038764208 | 0.027654729 | 0.160998649; 27 | SLE
GWAS | 9 | Myocardial infarction || id: ieu-a-798 | Inverse variance
weighted | 7 | 0.093340843 | 0.095494499 | 0.328347739; 28 | SLE
GWAS | 11 | Myocardial infarction || id: ieu-a-798 | Inverse variance
weighted | 9 | 0.045943819 | 0.063357101 | 0.468355973; 29 | SLE
GWAS | 15 | Myocardial infarction || id: ieu-a-798 | Inverse variance
weighted | 13 | 0.028512344 | 0.041789144 | 0.495055158; 30 | SLE
GWAS | 10 | Myocardial infarction || id: ieu-a-798 | Inverse variance
weighted | 6 | 0.054804983 | 0.053176893 | 0.302720696; 31 | SLE
GWAS | 14 | Myocardial infarction || id: ieu-a-798 | Inverse variance
weighted | 11 | 0.090695162 | 0.048175193 | 0.059753067; 32 | SLE
GWAS | 5 | Myocardial infarction || id: ieu-a-798 | Inverse variance
weighted | 4 | 0.127948285 | 0.156990182 | 0.415067617; 33 | SLE
GWAS | 51 Myocardial infarction || id: ieu-a-798 | Inverse variance weighted | 45
| −0.014082278 | 0.018965006 | 0.457760144; 34 | SLE GWAS | 31 | Myocardial infarction ||
id: ieu-a-798 | Inverse variance weighted | 25 | −0.029192218 | 0.016821321 | 0.082664744;
35 | SLE GWAS | 19 | Myocardial infarction || id: ieu-a-798 | Inverse variance
weighted | 16 | 0.073403782 | 0.021123853 | 0.000510998; 36 | SLE
GWAS | 2 | Myocardial infarction || id: ieu-a-798 | Inverse variance
weighted | 2 | 0.340221688 | 0.859478056 | 0.692218007; 37 | SLE GWAS | 9 | Myocardial
infarction || id: ieu-a-798 | Inverse variance weighted | 7
| −0.033233383 | 0.075307582 | 0.658994413; 38 | SLE GWAS | 10 | Myocardial infarction ||
id: ieu-a-798 | Inverse variance weighted | 10 | 0.036190544 | 0.043046457 | 0.400497963;
39 | SLE GWAS | 7 | Myocardial infarction || id: ieu-a-798 | Inverse variance weighted | 7
| −0.038085333 | 0.048954971 | 0.436588691; 40 | SLE GWAS | 37 | Myocardial infarction ||
id: ieu-a-798 | Inverse variance weighted | 33 | −0.06275901 | 0.011677472 | 7.68525E−08;
41 | SLE GWAS | 5 | Myocardial infarction || id: ieu-a-798 | Inverse variance weighted | 4
| −0.021521623 | 0.07991155 | 0.787684945; 42 | SLE GWAS | 79 | Myocardial infarction ||
id: ieu-a-798 | Inverse variance weighted | 65 | −0.00057294 | 0.01226115 | 0.962729923;
43 | SLE GWAS | 8 | Myocardial infarction || id: ieu-a-798 | Inverse variance
weighted | 8 | 0.059527644 | 0.036071181 | 0.098885143; 44 | SLE
GWAS | 21 | Myocardial infarction || id: ieu-a-798 | Inverse variance
weighted | 17 | 0.124780922 | 0.019895453 | 3.56827E−10; 45 | SLE
GWAS | 43 | Myocardial infarction || id: ieu-a-798 | Inverse variance weighted | 40
| −0.036533386 | 0.012187811 | 0.002721729; 46 | SLE GWAS | 73 | Myocardial infarction ||
id: ieu-a-798 | Inverse variance weighted | 64 | −0.043076499 | 0.011111457 | 0.000105855;

TABLE 7B
67 PPI-based cluser MR analysis (FIG. 10B). Listed by: Cluster | Exposure_data |
number of SNPs linking to each cluster | Outcome | MR method | # SNPs used as IVs | beta | SE | pval

1 | SLE GWAS | 133 | Atrial fibrillation ∥ id: ebi-a-GCST006414 | Inverse variance
weighted | 115 | −0.010495326 | 0.005484119 | 0.055649921; 2 | SLE GWAS | 191 | Atrial
fibrillation ∥ id: ebi-a-GCST006414 | Inverse variance weighted | 168 | −0.021484581 |
0.004952327 | 1.43602E−05; 3 | SLE GWAS | 62 | Atrial fibrillation ∥ id: ebi-a-GCST006414 |
Inverse variance
weighted | 46 | 0.010383126 | 0.004840352 | 0.03194343; 4 | SLE GWAS | 95 | Atrial
fibrillation ∥ id: ebi-a-GCST006414 | Inverse variance weighted | 84 | −0.00468301
| 0.008770175 | 0.593362318; 5 | SLE GWAS | 143 | Atrial fibrillation ∥ id: ebi-
a-GCST006414 | Inverse variance
weighted | 124 | 0.001517407 | 0.003897343 | 0.697021789; 6 | SLE GWAS | 61 | Atrial
fibrillation ∥ id: ebi-a-GCST006414 | Inverse variance weighted | 51 | −0.020706365 |
0.009659161 | 0.032056743; 7 | SLE GWAS | 56 | Atrial fibrillation ∥ id: ebi-
a-GCST006414 | Inverse variance
weighted | 50 | 0.001806026 | 0.008941813 | 0.83993597; 8 | SLE GWAS | 22 | Atrial
fibrillation ∥ id: ebi-a-GCST006414 | Inverse variance
weighted | 18 | 0.003120612 | 0.012002676 | 0.794869179; 9 | SLE GWAS | 264 | Atrial
fibrillation ∥ id: ebi-a-GCST006414 | Inverse variance weighted | 231 | −0.008022737 |
0.004081991 | 0.049368178; 10 | SLE GWAS | 94 | Atrial fibrillation ∥
id: ebi-a-GCST006414 | Inverse variance weighted | 82 | −0.056060755 |
0.007893597 | 1.22916E−12; 11 | SLE GWAS | 16 | Atrial fibrillation ∥
id: ebi-a-GCST006414 | Inverse variance weighted | 14 | −0.023072007 |
0.017442724 | 0.185925326; 12 | SLE GWAS | 56 | Atrial fibrillation ∥
id: ebi-a-GCST006414 | Inverse variance
weighted | 47 | 0.001750839 | 0.015670165 | 0.911036925; 13 | SLE GWAS | 44 | Atrial
fibrillation ∥ id: ebi-a-GCST006414 | Inverse variance weighted | 37 | −0.025190682 |
0.008462859 | 0.002914489; 14 | SLE GWAS | 96 | Atrial fibrillation ∥
id: ebi-a-GCST006414 | Inverse variance
weighted | 81 | 0.040554315 | 0.011955478 | 0.000693562; 15 | SLE GWAS | 6 | Atrial
fibrillation ∥ id: ebi-a-GCST006414 | Inverse variance weighted | 4 | −0.031709677 |
0.046556439 | 0.495807044; 16 | SLE GWAS | 265 | Atrial fibrillation ∥
id: ebi-a-GCST006414 | Inverse variance weighted | 233 | −0.023131561 |
0.005098516 | 5.70815E−06; 17 | SLE GWAS | 28 | Atrial fibrillation ∥
id: ebi-a-GCST006414 | Inverse variance
weighted | 22 | 0.034780772 | 0.013010381 | 0.0075107; 18 | SLE GWAS | 27 | Atrial
fibrillation ∥ id: ebi-a-GCST006414 | Inverse variance
weighted | 25 | 0.02579822 | 0.020366303 | 0.20525865; 19 | SLE GWAS | 216 | Atrial
fibrillation ∥ id: ebi-a-GCST006414 | Inverse variance weighted | 184 | −0.004893557 |
0.005666246 | 0.387789593; 20 | SLE GWAS | 40 | Atrial fibrillation ∥
id: ebi-a-GCST006414 | Inverse variance
weighted | 34 | 0.000846279 | 0.006015445 | 0.888119265; 21 | SLE GWAS | 133 | Atrial
fibrillation ∥ id: ebi-a-GCST006414 | Inverse variance weighted | 111 | −0.049674276 |
0.007249467 | 7.27593E−12; 22 | SLE GWAS | 10 | Atrial fibrillation ∥
id: ebi-a-GCST006414 | Inverse variance weighted | 8 | −0.220351434 |
0.059209495 | 0.000197999; 23 | SLE GWAS | 14 | Atrial fibrillation ∥
id: ebi-a-GCST006414 | Inverse variance
weighted | 13 | 0.029625031 | 0.020631779 | 0.151032742; 24 | SLE GWAS | 27 | Atrial
fibrillation ∥ id: ebi-a-GCST006414 | Inverse variance weighted | 20 | −0.145639897 |
0.011468112 | 5.94687E−37; 25 | SLE GWAS | 51 | Atrial fibrillation ∥
id: ebi-a-GCST006414 | Inverse variance weighted | 42 | −0.054606348 |
0.017368042 | 0.001666153; 27 | SLE GWAS | 28 | Atrial fibrillation ∥
id: ebi-a-GCST006414 | Inverse variance
weighted | 19 | 0.004061646 | 0.018921317 | 0.830032556; 28 | SLE GWAS | 27 | Atrial
fibrillation ∥ id: ebi-a-GCST006414 | Inverse variance weighted | 23 | −0.004989438 |
0.03203924 | 0.876246659; 29 | SLE GWAS | 12 | Atrial fibrillation ∥ id: ebi-
a-GCST006414 | Inverse variance
weighted | 9 | 0.081082849 | 0.032082255 | 0.011492934; 30 | SLE GWAS | 69 | Atrial
fibrillation ∥ id: ebi-a-GCST006414 | Inverse variance weighted | 59 | −0.052414076 |
0.008767524 | 2.25607E−09; 31 | SLE GWAS | 19 | Atrial fibrillation ∥
id: ebi-a-GCST006414 | Inverse variance weighted | 16 | −0.032536226 |
0.025180806 | 1.96E−01; 32 | SLE GWAS | 26 | Atrial fibrillation ∥ id: ebi-a-
GCST006414 | Inverse variance weighted | 22 | 0.029939131 | 0.011302012 | 0.00807282;
33 | SLE GWAS | 41 | Atrial fibrillation ∥ id: ebi-a-GCST006414 | Inverse variance
weighted | 36 | 0.010277073 | 0.006273148 | 0.101366598; 34 | SLE GWAS | 8 | Atrial
fibrillation ∥ id: ebi-a-GCST006414 | Inverse variance
weighted | 8 | 0.063609464 | 0.016269424 | 9.23897E−05; 35 | SLE GWAS | 163 | Atrial
fibrillation ∥ id: ebi-a-GCST006414 | Inverse variance weighted | 140 | −0.013981253 |
0.00466893 | 0.002748679; 36 | SLE GWAS | 59 | Atrial fibrillation ∥ id: ebi-
a-GCST006414 | Inverse variance weighted | 46 | −0.120655389 | 0.007886896 | 7.85895E−53;
37 | SLE GWAS | 2 | Atrial fibrillation ∥ id: ebi-a-GCST006414 | Inverse variance
weighted | 2 | 0.031328074 | 0.34857976 | 0.92838776; 38 | SLE GWAS | 15 | Atrial
fibrillation ∥ id: ebi-a-GCST006414 | Inverse variance
weighted | 14 | 0.016087437 | 0.030454978 | 0.597335068; 39 | SLE GWAS | 12 | Atrial
fibrillation ∥ id: ebi-a-GCST006414 | Inverse variance weighted | 10 | −0.005982442 |
0.022890973 | 0.793826434; 40 | SLE GWAS | 4 | Atrial fibrillation ∥ id: ebi-
a-GCST006414 | Inverse variance weighted | 4 | −0.263843812 |
0.096019023 | 0.005999137; 41 | SLE GWAS | 19 | Atrial fibrillation ∥
id: ebi-a-GCST006414 | Inverse variance weighted | 18 | 0.0746219 | 0.037217931 |
4.50E−02; 42 | SLE GWAS | 23 | Atrial fibrillation ∥ id: ebi-a-GCST006414 | Inverse variance
weighted | 20 | −0.022845085 | 0.035178182 | 0.516073008; 43 | SLE GWAS | 27 | Atrial
fibrillation ∥ id: ebi-a-GCST006414 | Inverse variance
weighted | 18 | 0.010960034 | 0.014977129 | 0.464299978; 44 | SLE GWAS | 36 | Atrial
fibrillation ∥ id: ebi-a-GCST006414 | Inverse variance
weighted | 35 | 0.009813443 | 0.00524089 | 0.061140723; 45 | SLE GWAS | 77 | Atrial
fibrillation ∥ id: ebi-a-GCST006414 | Inverse variance weighted | 71 | −0.029750472 |
0.005201591 | 1.06841E−08; 46 | SLE GWAS | 8 | Atrial fibrillation ∥ id: ebi-
a-GCST006414 | Inverse variance weighted | 6 | 0.010368902 | 0.04974323 | 0.834878778;
47 | SLE GWAS | 16 | Atrial fibrillation ∥ id: ebi-a-GCST006414 | Inverse variance
weighted | 14 | −0.045524435 | 0.04035145 | 2.59E−01; 48 | SLE GWAS | 3 | Atrial
fibrillation ∥ id: ebi-a-GCST006414 | Inverse variance
weighted | 3 | 0.00983086 | 0.178968697 | 9.56E−01; 49 | SLE GWAS | 32 | Atrial
fibrillation ∥ id: ebi-a-GCST006414 | Inverse variance weighted | 27 | −0.003888172 |
0.018551828 | 8.34E−01; 50 | SLE GWAS | 39 | Atrial fibrillation ∥ id: ebi-a-
GCST006414 | Inverse variance weighted | 35 | −0.019881317 |
0.006820762 | 0.003558898; 51 | SLE GWAS | 5 | Atrial fibrillation ∥ id: ebi-
a-GCST006414 | Inverse variance weighted | 4 | −0.00490839 |
0.050980874 | 0.923298952; 52 | SLE GWAS | 8 | Atrial fibrillation ∥ id: ebi-
a-GCST006414 | Inverse variance
weighted | 8 | 0.019733489 | 0.026540569 | 0.457165885; 53 | SLE GWAS | 9 | Atrial
fibrillation ∥ id: ebi-a-GCST006414 | Inverse variance
weighted | 7 | 0.028697301 | 0.065932012 | 0.663376654; 54 | SLE GWAS | 4 | Atrial
fibrillation ∥ id: ebi-a-GCST006414 | Inverse variance weighted | 4 | −0.018056015 |
0.046563213 | 6.98E−01; 55 | SLE GWAS | 27 | Atrial fibrillation ∥ id: ebi-a-
GCST006414 | Inverse variance weighted | 25 | 0.008207964 | 0.016742945 | 0.623968187;
56 | SLE GWAS | 19 | Atrial fibrillation ∥ id: ebi-a-GCST006414 | Inverse variance
weighted | 17 | 0.010143599 | 0.019666075 | 0.605999852; 57 | SLE GWAS | 27 | Atrial
fibrillation ∥ id: ebi-a-GCST006414 | Inverse variance
weighted | 25 | 0.018208298 | 0.015715155 | 0.246600592; 58 | SLE GWAS | 10 | Atrial
fibrillation ∥ id: ebi-a-GCST006414 | Inverse variance
weighted | 8 | 0.011551556 | 0.060032305 | 0.847411402; 59 | SLE GWAS | 8 | Atrial
fibrillation ∥ id: ebi-a-GCST006414 | Inverse variance
weighted | 8 | 0.089107682 | 0.05014878 | 0.075590228; 60 | SLE GWAS | 4 | Atrial
fibrillation ∥ id: ebi-a-GCST006414 | Inverse variance weighted | 4 | −0.265013173 |
0.15895882 | 0.095478358; 61 | SLE GWAS | 79 | Atrial fibrillation ∥ id: ebi-
a-GCST006414 | Inverse variance
weighted | 71 | 0.021585824 | 0.013505579 | 0.109978589; 62 | SLE GWAS | 7 | Atrial
fibrillation ∥ id: ebi-a-GCST006414 | Inverse variance weighted | 7 | −0.021430415 |
0.046460359 | 0.644610392; 63 | SLE GWAS | 14 | Atrial fibrillation ∥
id: ebi-a-GCST006414 | Inverse variance weighted | 12 | −0.020409945 |
0.0265661 | 0.442326641; 64 | SLE GWAS | 35 | Atrial fibrillation ∥ id: ebi-
a-GCST006414 | Inverse variance weighted | 32 | −0.019244433 |
0.006868438 | 0.005080822; 65 | SLE GWAS | 22 | Atrial fibrillation ∥
id: ebi-a-GCST006414 | Inverse variance
weighted | 18 | 0.002420339 | 0.013755241 | 0.860327275; 66 | SLE GWAS | 7 | Atrial
fibrillation ∥ id: ebi-a-GCST006414 | Inverse variance
weighted | 6 | 0.051306178 | 0.073795123 | 0.486897639; 67 | SLE GWAS | 17 | Atrial
fibrillation ∥ id: ebi-a-GCST006414 | Inverse variance
weighted | 14 | 0.030580243 | 0.014057926 | 0.029607449; 1 | SLE
GWAS | 133 | Cardiomyopathy ∥ id: finn-b-I9_CARDMYO | Inverse variance
weighted | 115 | −0.041801392 | 0.017374047 | 0.0161297; 2 | SLE
GWAS | 191 | Cardiomyopathy ∥ id: finn-b-I9_CARDMYO | Inverse variance
weighted | 167 | −0.030476387 | 0.012712701 | 0.016515581; 3 | SLE
GWAS | 62 | Cardiomyopathy ∥ id: finn-b-I9_CARDMYO | Inverse variance
weighted | 45 | −0.05262349 | 0.017476635 | 0.00260323; 4 | SLE
GWAS | 95 | Cardiomyopathy ∥ id: finn-b-I9_CARDMYO | Inverse variance
weighted | 83 | 0.003727973 | 0.02622326 | 0.886951387; 5 | SLE
GWAS | 143 | Cardiomyopathy ∥ id: finn-b-I9_CARDMYO | Inverse variance
weighted | 123 | −0.02822799 | 0.014510585 | 0.051734324; 6 | SLE
GWAS | 61 | Cardiomyopathy ∥ id: finn-b-I9_CARDMYO | Inverse variance
weighted | 51 | −0.137847993 | 0.023285061 | 3.21905E−09; 7 | SLE
GWAS | 56 | Cardiomyopathy ∥ id: finn-b-I9_CARDMYO | Inverse variance
weighted | 50 | 0.024586089 | 0.028259466 | 0.384293565; 8 | SLE
GWAS | 22 | Cardiomyopathy ∥ id: finn-b-I9_CARDMYO | Inverse variance
weighted | 18 | −0.017872573 | 0.047879488 | 0.708938233; 9 | SLE
GWAS | 264 | Cardiomyopathy ∥ id: finn-b-I9_CARDMYO | Inverse variance
weighted | 230 | −0.024420636 | 0.009074977 | 0.007124123; 10 | SLE
GWAS | 94 | Cardiomyopathy ∥ id: finn-b-I9_CARDMYO | Inverse variance
weighted | 82 | 0.058675721 | 0.026643115 | 0.027645214; 11 | SLE
GWAS | 16 | Cardiomyopathy ∥ id: finn-b-I9_CARDMYO | Inverse variance
weighted | 14 | −0.073161273 | 0.058418537 | 0.210437255; 12 | SLE
GWAS | 56 | Cardiomyopathy ∥ id: finn-b-I9_CARDMYO | Inverse variance
weighted | 47 | 0.149980696 | 0.052240111 | 0.00409192; 13 | SLE
GWAS | 44 | Cardiomyopathy ∥ id: finn-b-I9_CARDMYO | Inverse variance
weighted | 37 | −0.162607121 | 0.034899133 | 3.17216E−06; 14 | SLE
GWAS | 96 | Cardiomyopathy ∥ id: finn-b-I9_CARDMYO | Inverse variance
weighted | 80 | 0.132879374 | 0.032414162 | 4.14181E−05; 15 | SLE
GWAS | 6 | Cardiomyopathy ∥ id: finn-b-I9_CARDMYO | Inverse variance weighted |
4 | −0.255863771 | 0.165000979 | 0.120978313; 16 | SLE GWAS | 265 | Cardiomyopathy ∥
id: finn-b-I9_CARDMYO | Inverse variance weighted | 233 | −0.103190935 |
0.012470911 | 1.28963E−16; 17 | SLE GWAS | 28 | Cardiomyopathy ∥
id: finn-b-I9_CARDMYO | Inverse variance
weighted | 23 | 0.040498204 | 0.056274657 | 0.471738903; 18 | SLE
GWAS | 27 | Cardiomyopathy ∥ id: finn-b-I9_CARDMYO | Inverse variance
weighted | 25 | −0.236369697 | 0.075888071 | 0.001841229; 19 | SLE
GWAS | 216 | Cardiomyopathy ∥ id: finn-b-I9_CARDMYO | Inverse variance
weighted | 184 | −0.048313798 | 0.016755603 | 0.003933559; 20 | SLE
GWAS | 40 | Cardiomyopathy ∥ id: finn-b-I9_CARDMYO | Inverse variance
weighted | 35 | −0.061738838 | 0.0247294 | 0.012539864; 21 | SLE
GWAS | 133 | Cardiomyopathy ∥ id: finn-b-I9_CARDMYO | Inverse variance
weighted | 111 | −0.037042173 | 0.020820576 | 0.075221115; 22 | SLE
GWAS | 10 | Cardiomyopathy ∥ id: finn-b-I9_CARDMYO | Inverse variance
weighted | 8 | 0.002347635 | 0.219463623 | 0.991465074; 23 | SLE
GWAS | 14 | Cardiomyopathy ∥ id: finn-b-I9_CARDMYO | Inverse variance
weighted | 12 | −0.253597765 | 0.128140893 | 0.047809911; 24 | SLE
GWAS | 27 | Cardiomyopathy ∥ id: finn-b-I9_CARDMYO | Inverse variance
weighted | 20 | −0.392701289 | 0.044322231 | 7.99121E−19; 25 | SLE
GWAS | 51 | Cardiomyopathy ∥ id: finn-b-I9_CARDMYO | Inverse variance
weighted | 42 | −0.227204489 | 0.03526395 | 1.1716E−10; 27 | SLE
GWAS | 28 | Cardiomyopathy ∥ id: finn-b-I9_CARDMYO | Inverse variance
weighted | 19 | 0.01152911 | 0.072490201 | 0.873634443; 28 | SLE
GWAS | 27 | Cardiomyopathy ∥ id: finn-b-I9_CARDMYO | Inverse variance
weighted | 23 | −0.013533512 | 0.103328757 | 0.895794867; 29 | SLE
GWAS | 12 | Cardiomyopathy ∥ id: finn-b-I9_CARDMYO | Inverse variance
weighted | 9 | 0.123898341 | 0.116221942 | 0.286401275; 30 | SLE
GWAS | 69 | Cardiomyopathy ∥ id: finn-b-I9_CARDMYO | Inverse variance
weighted | 59 | −0.061342347 | 0.032773808 | 0.06124976; 31 | SLE
GWAS | 19 | Cardiomyopathy ∥ id: finn-b-I9_CARDMYO | Inverse variance
weighted | 16 | 0.132181363 | 0.079523174 | 0.096477865; 32 | SLE
GWAS | 26 | Cardiomyopathy ∥ id: finn-b-I9_CARDMYO | Inverse variance
weighted | 22 | 0.042892983 | 0.045712344 | 0.348078006; 33 | SLE
GWAS | 41 | Cardiomyopathy ∥ id: finn-b-I9_CARDMYO | Inverse variance
weighted | 36 | −0.064432658 | 0.025750593 | 0.012343062; 34 | SLE
GWAS | 8 | Cardiomyopathy ∥ id: finn-b-I9_CARDMYO | Inverse variance
weighted | 8 | 0.038200301 | 0.061108469 | 0.531890419; 35 | SLE
GWAS | 163 | Cardiomyopathy ∥ id: finn-b-I9_CARDMYO | Inverse variance
weighted | 140 | −0.00904256 | 0.010510486 | 0.389603301; 36 | SLE
GWAS | 59 | Cardiomyopathy ∥ id: finn-b-I9_CARDMYO | Inverse variance
weighted | 46 | 0.118033357 | 0.053359931 | 0.026965125; 37 | SLE
GWAS | 2 | Cardiomyopathy ∥ id: finn-b-I9_CARDMYO | Inverse variance weighted | 2
| −0.009948161 | 0.598474523 | 0.98673775; 38 | SLE GWAS | 15 | Cardiomyopathy ∥
id: finn-b-I9_CARDMYO | Inverse variance
weighted | 14 | 0.172847266 | 0.101395859 | 0.088254538; 39 | SLE
GWAS | 12 | Cardiomyopathy ∥ id: finn-b-I9_CARDMYO | Inverse variance
weighted | 10 | 0.07417093 | 0.076128247 | 0.329912959; 40 | SLE
GWAS | 4 | Cardiomyopathy ∥ id: finn-b-I9_CARDMYO | Inverse variance
weighted | 4 | 0.458847939 | 0.283275895 | 0.105277033; 41 | SLE
GWAS | 19 | Cardiomyopathy ∥ id: finn-b-I9_CARDMYO | Inverse variance
weighted | 18 | 0.00532404 | 0.109674772 | 0.961282785; 42 | SLE
GWAS | 23 | Cardiomyopathy ∥ id: finn-b-I9_CARDMYO | Inverse variance
weighted | 21 | −0.038779173 | 0.110164149 | 0.724828782; 43 | SLE
GWAS | 27 | Cardiomyopathy ∥ id: finn-b-I9_CARDMYO | Inverse variance
weighted | 18 | 0.057505243 | 0.071621473 | 0.422029618; 44 | SLE
GWAS | 36 | Cardiomyopathy ∥ id: finn-b-I9_CARDMYO | Inverse variance
weighted | 35 | −0.066055257 | 0.021515859 | 0.002140064; 45 | SLE
GWAS | 77 | Cardiomyopathy ∥ id: finn-b-I9_CARDMYO | Inverse variance
weighted | 71 | 0.014526973 | 0.022638319 | 0.521069029; 46 | SLE
GWAS | 8 | Cardiomyopathy ∥ id: finn-b-I9_CARDMYO | Inverse variance
weighted | 6 | 0.08446906 | 0.134016564 | 0.528506037; 47 | SLE
GWAS | 16 | Cardiomyopathy ∥ id: finn-b-I9_CARDMYO | Inverse variance
weighted | 14 | −0.03653023 | 0.083274418 | 0.66089857; 48 | SLE
GWAS | 3 | Cardiomyopathy ∥ id: finn-b-I9_CARDMYO | Inverse variance weighted | 3
| −0.334610837 | 0.474174154 | 0.480392907; 49 | SLE GWAS | 32 | Cardiomyopathy ∥
id: finn-b-I9_CARDMYO | Inverse variance
weighted | 27 | 0.049965479 | 0.056893538 | 0.379820181; 50 | SLE
GWAS | 39 | Cardiomyopathy ∥ id: finn-b-I9_CARDMYO | Inverse variance
weighted | 35 | 0.072770575 | 0.030055393 | 0.015468717; 51 | SLE
GWAS | 5 | Cardiomyopathy ∥ id: finn-b-I9_CARDMYO | Inverse variance weighted
| 4 | −0.319410546 | 0.452796519 | 0.480550516; 52 | SLE GWAS | 8 | Cardiomyopathy ∥
id: finn-b-I9_CARDMYO | Inverse variance weighted | 8 | −0.321790865 |
0.113354139 | 0.004528211; 53 | SLE GWAS | 9 | Cardiomyopathy ∥
id: finn-b-I9_CARDMYO | Inverse variance
weighted | 7 | 0.288127297 | 0.155681935 | 0.064206492; 54 | SLE
GWAS | 4 | Cardiomyopathy ∥ id: finn-b-I9_CARDMYO | Inverse variance
weighted | 4 | 0.036748725 | 0.199022681 | 0.853506769; 55 | SLE
GWAS | 27 | Cardiomyopathy ∥ id: finn-b-I9_CARDMYO | Inverse variance
weighted | 25 | 0.004801676 | 0.055562105 | 0.931132571; 56 | SLE
GWAS | 19 | Cardiomyopathy ∥ id: finn-b-I9_CARDMYO | Inverse variance
weighted | 17 | −0.12444787 | 0.091343083 | 0.173064592; 57 | SLE
GWAS | 27 | Cardiomyopathy ∥ id: finn-b-I9_CARDMYO | Inverse variance
weighted | 25 | −0.080799169 | 0.05021212 | 0.107582085; 58 | SLE
GWAS | 10 | Cardiomyopathy ∥ id: finn-b-I9_CARDMYO | Inverse variance
weighted | 8 | 0.414740951 | 0.181994763 | 0.022675269; 59 | SLE
GWAS | 8 | Cardiomyopathy ∥ id: finn-b-I9_CARDMYO | Inverse variance weighted
| 8 | −0.338052532 | 0.222076351 | 0.127950053; 60 | SLE GWAS | 4 | Cardiomyopathy ∥
id: finn-b-I9_CARDMYO | Inverse variance
weighted | 4 | 0.091377015 | 0.277993291 | 0.742380767; 61 | SLE
GWAS | 79 | Cardiomyopathy ∥ id: finn-b-I9_CARDMYO | Inverse variance
weighted | 71 | −0.24229455 | 0.021840586 | 1.34489E−28; 62 | SLE
GWAS | 7 | Cardiomyopathy ∥ id: finn-b-I9_CARDMYO | Inverse variance weighted
| 7 | −0.053605651 | 0.098959155 | 0.588028836; 63 | SLE GWAS | 14 | Cardiomyopathy ∥
id: finn-b-I9_CARDMYO | Inverse variance weighted | 12 | −0.087719084
| 0.064156506 | 0.17154158; 64 | SLE GWAS | 35 | Cardiomyopathy ∥
id: finn-b-I9_CARDMYO | Inverse variance
weighted | 32 | 0.081790111 | 0.030745435 | 0.007808593; 65 | SLE
GWAS | 22 | Cardiomyopathy ∥ id: finn-b-I9_CARDMYO | Inverse variance
weighted | 18 | 0.060271081 | 0.056965936 | 0.290046467; 66 | SLE
GWAS | 7 | Cardiomyopathy ∥ id: finn-b-I9_CARDMYO | Inverse variance weighted
| 6 | −0.14086722 | 0.224194995 | 0.52979135; 67 | SLE GWAS | 17 | Cardiomyopathy ∥ id: finn-
b-19_CARDMYO | Inverse variance
weighted | 14 | 0.048081688 | 0.058295677 | 0.409490861; 1 | SLE GWAS | 133 | Coronary
artery disease ∥ id: ebi-a-GCST005194 | Inverse variance weighted | 115 | −0.020597271 |
0.006362712 | 0.001207154; 2 | SLE GWAS | 191 | Coronary artery disease ∥
id: ebi-a-GCST005194 | Inverse variance
weighted | 168 | 0.016421129 | 0.002821886 | 5.91286E−09; 3 | SLE GWAS | 62 | Coronary
artery disease ∥ id: ebi-a-GCST005194 | Inverse variance
weighted | 46 | 0.021807228 | 0.003613444 | 1.58938E−09; 4 | SLE GWAS | 95 | Coronary
artery disease ∥ id: ebi-a-GCST005194 | Inverse variance
weighted | 84 | 0.031628601 | 0.009769575 | 0.001205992; 5 | SLE GWAS | 143 | Coronary
artery disease ∥ id: ebi-a-GCST005194 | Inverse variance
weighted | 124 | 0.012656512 | 0.004200845 | 0.002588079; 6 | SLE GWAS | 61 | Coronary
artery disease ∥ id: ebi-a-GCST005194 | Inverse variance weighted | 51 | −0.004815657 |
0.009836941 | 0.624453619; 7 | SLE GWAS | 56 | Coronary artery disease ∥
id: ebi-a-GCST005194 | Inverse variance weighted | 50 | −0.004857434 |
0.009097634 | 0.59339497; 8 | SLE GWAS | 22 | Coronary artery disease ∥
id: ebi-a-GCST005194 | Inverse variance
weighted | 18 | 0.048969037 | 0.010405884 | 2.5275E−06; 9 | SLE GWAS | 264 | Coronary
artery disease ∥ id: ebi-a-GCST005194 | Inverse variance
weighted | 231 | 0.004275448 | 0.002560797 | 0.095003068; 10 | SLE
GWAS | 94 | Coronary artery disease ∥ id: ebi-a-GCST005194 | Inverse variance
weighted | 82 −0.029604363 | 0.008008113 | 0.000218337; 11 | SLE
GWAS | 16 | Coronary artery disease ∥ id: ebi-a-GCST005194 | Inverse variance
weighted | 14 | 0.021283189 | 0.029515099 | 0.470851091; 12 | SLE GWAS | 56 | Coronary
artery disease ∥ id: ebi-a-GCST005194 | Inverse variance
weighted | 47 | 0.048112414 | 0.02372673 | 0.042583457; 13 | SLE GWAS | 44 | Coronary
artery disease ∥ id: ebi-a-GCST005194 | Inverse variance
weighted | 37 | 0.031121478 | 0.008379926 | 0.00020416; 14 | SLE GWAS | 96 | Coronary
artery disease ∥ id: ebi-a-GCST005194 | Inverse variance
weighted | 81 | 0.023879493 | 0.01129836 | 0.034554909; 15 | SLE GWAS | 6 | Coronary
artery disease ∥ id: ebi-a-GCST005194 | Inverse variance
weighted | 4 | 0.081469907 | 0.039192013 | 0.037641485; 16 | SLE GWAS | 265 | Coronary
artery disease ∥ id: ebi-a-GCST005194 | Inverse variance
weighted | 233 | 0.006117102 | 0.00340734 | 0.072610403; 17 | SLE GWAS | 28 | Coronary
artery disease ∥ id: ebi-a-GCST005194 | Inverse variance
weighted | 22 | 0.020857404 | 0.014605463 | 0.153276049; 18 | SLE GWAS | 27 | Coronary
artery disease ∥ id: ebi-a-GCST005194 | Inverse variance
weighted | 25 | 0.081021574 | 0.035827378 | 0.023731838; 19 | SLE
GWAS | 216 | Coronary artery disease ∥ id: ebi-a-GCST005194 | Inverse variance
weighted | 183 | 0.039440412 | 0.005311997 | 1.12967E−13; 20 | SLE
GWAS | 40 | Coronary artery disease ∥ id: ebi-a-GCST005194 | Inverse variance
weighted | 33 | 0.008150189 | 0.00586579 | 0.164697634; 21 | SLE GWAS | 133 | Coronary
artery disease ∥ id: ebi-a-GCST005194 | Inverse variance
weighted | 111 | 0.021756255 | 0.003691773 | 3.78852E−09; 22 | SLE
GWAS | 10 | Coronary artery disease ∥ id: ebi-a-GCST005194 | Inverse variance
weighted | 8 | 0.069530278 | 0.042707716 | 0.103514382; 23 | SLE GWAS | 14 | Coronary
artery disease ∥ id: ebi-a-GCST005194 | Inverse variance
weighted | 13 | 0.076985929 | 0.030944868 | 0.012852096; 24 | SLE GWAS | 27 | Coronary
artery disease ∥ id: ebi-a-GCST005194 | Inverse variance weighted | 20 | −0.127415345 |
0.010019122 | 4.74461E−37; 25 | SLE GWAS | 51 | Coronary artery disease
∥ id: ebi-a-GCST005194 | Inverse variance weighted | 42 | −0.036975651 |
0.014893242 | 0.013038594; 27 | SLE GWAS | 28 | Coronary artery disease
∥ id: ebi-a-GCST005194 | Inverse variance
weighted | 19 | 0.035039446 | 0.020434769 | 0.086400709; 28 | SLE GWAS | 27 | Coronary
artery disease ∥ id: ebi-a-GCST005194 | Inverse variance
weighted | 23 | 0.133120243 | 0.044894008 | 0.003024746; 29 | SLE GWAS | 12 | Coronary
artery disease ∥ id: ebi-a-GCST005194 | Inverse variance weighted | 9 | −0.039408903 |
0.051085507 | 0.440451978; 30 | SLE GWAS | 69 | Coronary artery disease
∥ id: ebi-a-GCST005194 | Inverse variance weighted | 59 | −0.077299804 |
0.007332911 | 5.56109E−26; 31 | SLE GWAS | 19 | Coronary artery disease
∥ id: ebi-a-GCST005194 | Inverse variance
weighted | 16 | 0.044657009 | 0.029030063 | 0.123974749; 32 | SLE GWAS | 26 | Coronary
artery disease ∥ id: ebi-a-GCST005194 | Inverse variance
weighted | 22 | 0.022272316 | 0.018569374 | 0.230368086; 33 | SLE GWAS | 41 | Coronary
artery disease ∥ id: ebi-a-GCST005194 | Inverse variance
weighted | 36 | 0.027780974 | 0.00721341 | 0.000117494; 34 | SLE GWAS | 8 | Coronary
artery disease ∥ id: ebi-a-GCST005194 | Inverse variance
weighted | 8 | 0.117004731 | 0.022564949 | 2.15734E−07; 35 | SLE GWAS | 163 | Coronary
artery disease ∥ id: ebi-a-GCST005194 | Inverse variance weighted | 140 | −0.001317224 |
0.002797366 | 0.637726315; 36 | SLE GWAS | 59 | Coronary artery disease
∥ id: ebi-a-GCST005194 | Inverse variance
weighted | 46 | 0.013128296 | 0.006559969 | 0.045362856; 37 | SLE GWAS | 2 | Coronary
artery disease ∥ id: ebi-a-GCST005194 | Inverse variance
weighted | 2 | 0.275873213 | 0.320243194 | 0.388990741; 38 | SLE GWAS | 15 | Coronary
artery disease ∥ id: ebi-a-GCST005194 | Inverse variance
weighted | 14 | 0.090973393 | 0.067606583 | 0.178422123; 39 | SLE GWAS | 12 | Coronary
artery disease ∥ id: ebi-a-GCST005194 | Inverse variance
weighted | 10 | 0.102379609 | 0.056630215 | 0.070627996; 40 | SLE GWAS | 4 | Coronary
artery disease ∥ id: ebi-a-GCST005194 | Inverse variance weighted | 4 | −0.198422102 |
0.065230605 | 0.002351244; 41 | SLE GWAS | 19 | Coronary artery disease ∥
id: ebi-a-GCST005194 | Inverse variance
weighted | 17 | 0.053283674 | 0.032610667 | 0.10227273; 42 | SLE GWAS | 23 | Coronary
artery disease ∥ id: ebi-a-GCST005194 | Inverse variance
weighted | 20 | 0.052672697 | 0.036493483 | 0.148923221; 43 | SLE GWAS | 27 | Coronary
artery disease ∥ id: ebi-a-GCST005194 | Inverse variance
weighted | 18 | 0.02845562 | 0.021165223 | 0.178802375; 44 | SLE GWAS | 36 | Coronary
artery disease ∥ id: ebi-a-GCST005194 | Inverse variance
weighted | 35 | 0.024230323 | 0.005117314 | 2.19088E−06; 45 | SLE GWAS | 77 | Coronary
artery disease ∥ id: ebi-a-GCST005194 | Inverse variance weighted | 71 | −0.038603069 |
0.004836351 | 1.44146E−15; 46 | SLE GWAS | 8 | Coronary artery disease ∥
id: ebi-a-GCST005194 | Inverse variance
weighted | 6 | 0.103388074 | 0.070190974 | 0.140763448; 47 | SLE GWAS | 16 | Coronary
artery disease ∥ id: ebi-a-GCST005194 | Inverse variance weighted | 14 | −0.026354801 |
0.022961924 | 0.251067237; 48 | SLE GWAS | 3 | Coronary artery disease ∥
id: ebi-a-GCST005194 | Inverse variance
weighted | 3 | 0.567463513 | 0.245872514 | 0.021001455; 49 | SLE GWAS | 32 | Coronary
artery disease ∥ id: ebi-a-GCST005194 | Inverse variance weighted | 27 | 1.0404E−05
| 0.019724438 | 0.99957914; 50 | SLE GWAS | 39 | Coronary artery disease ∥ id: ebi-a-
GCST005194 | Inverse variance weighted | 35 | −0.062716572 | 0.005836664 | 6.23744E−27;
51 | SLE GWAS | 5 | Coronary artery disease ∥ id: ebi-a-GCST005194 | Inverse
variance weighted | 4 | 0.12276036 | 0.046998289 | 0.00900096; 52 | SLE
GWAS | 8 | Coronary artery disease ∥ id: ebi-a-GCST005194 | Inverse variance
weighted | 8 | 0.003772197 | 0.025975282 | 0.884535166; 53 | SLE GWAS | 9 | Coronary
artery disease ∥ id: ebi-a-GCST005194 | Inverse variance
weighted | 7 | 0.008760306 | 0.033890588 | 0.796030399; 54 | SLE GWAS | 4 | Coronary
artery disease ∥ id: ebi-a-GCST005194 | Inverse variance weighted | 4 | −0.004204122
| 0.038213587 | 0.912396355; 55 | SLE GWAS | 27 | Coronary artery disease
∥ id: ebi-a-GCST005194 | Inverse variance
weighted | 25 | 0.041391273 | 0.020953062 | 0.048219552; 56 | SLE GWAS | 19 | Coronary
artery disease ∥ id: ebi-a-GCST005194 | Inverse variance weighted | 16 | −0.036688513
| 0.022159142 | 0.097786051; 57 | SLE GWAS | 27 | Coronary artery disease
∥ id: ebi-a-GCST005194 | Inverse variance
weighted | 25 | 0.032334522 | 0.022722192 | 0.154725399; 58 | SLE GWAS | 10 | Coronary
artery disease ∥ id: ebi-a-GCST005194 | Inverse variance
weighted | 8 | 0.019084815 | 0.070203235 | 0.78573664; 59 | SLE GWAS | 8 | Coronary
artery disease ∥ id: ebi-a-GCST005194 | Inverse variance
weighted | 8 | 0.05249497 | 0.055716111 | 0.346097141; 60 | SLE GWAS | 4 | Coronary
artery disease ∥ id: ebi-a-GCST005194 | Inverse variance
weighted | 4 | 0.212939365 | 0.077515063 | 0.006013016; 61 | SLE GWAS | 79 | Coronary
artery disease ∥ id: ebi-a-GCST005194 | Inverse variance weighted | 71 | −0.0174862
| 0.004621084 | 0.000154325; 62 | SLE GWAS | 7 | Coronary artery disease ∥
id: ebi-a-GCST005194 | Inverse variance weighted | 7 | -
0.023287087 | 0.031159985 | 0.454858653; 63 | SLE GWAS | 14 | Coronary artery disease
∥ id: ebi-a-GCST005194 | Inverse variance weighted | 12 | −0.000112257
| 0.029910532 | 0.997005483; 64 | SLE GWAS | 35 | Coronary artery disease
∥ id: ebi-a-GCST005194 | Inverse variance weighted | 32 | −0.06103238
| 0.005867262 | 2.42297E−25; 65 | SLE GWAS | 22 | Coronary artery disease ∥
id: ebi-a-GCST005194 | Inverse variance
weighted | 18 | 0.044328019 | 0.011295497 | 8.69472E−05; 66 | SLE GWAS | 7 | Coronary
artery disease ∥ id: ebi-a-GCST005194 | Inverse variance weighted | 6 | -
0.163102892 | 0.05993631 | 0.006503159; 67 | SLE GWAS | 17 | Coronary artery disease ∥
id: ebi-a-GCST005194 | Inverse variance
weighted | 14 | 0.073253914 | 0.034466081 | 0.033553966; 1 | SLE GWAS | 133 | Coronary
artery disease ∥ id: ebi-a-GCST005195 | Inverse variance weighted | 115 | −0.020050484
| 0.006728524 | 0.002883206; 2 | SLE GWAS | 191 | Coronary artery disease
∥ id: ebi-a-GCST005195 | Inverse variance
weighted | 168 | 0.009360899 | 0.003202255 | 0.003464302; 3 | SLE GWAS | 62 | Coronary
artery disease ∥ id: ebi-a-GCST005195 | Inverse variance
weighted | 46 | 0.033566569 | 0.004473618 | 6.2266E−14; 4 | SLE GWAS | 95 | Coronary
artery disease ∥ id: ebi-a-GCST005195 | Inverse variance
weighted | 84 | 0.024179209 | 0.010416279 | 0.020271171; 5 | SLE GWAS | 143 | Coronary
artery disease ∥ id: ebi-a-GCST005195 | Inverse variance
weighted | 124 | 0.01411348 | 0.004456344 | 0.00153992; 6 | SLE GWAS | 61 | Coronary
artery disease ∥ id: ebi-a-GCST005195 | Inverse variance weighted | 51 | −0.013318054 |
0.011924444 | 0.264049918; 7 | SLE GWAS | 56 | Coronary artery disease ∥
id: ebi-a-GCST005195 | Inverse variance
weighted | 50 | 0.002071326 | 0.009695173 | 0.830823863; 8 | SLE GWAS | 22 | Coronary
artery disease ∥ id: ebi-a-GCST005195 | Inverse variance
weighted | 18 | 0.035768646 | 0.011963197 | 0.002790776; 9 | SLE GWAS | 264 | Coronary
artery disease ∥ id: ebi-a-GCST005195 | Inverse variance weighted | 231 | −0.000189327 |
0.002463558 | 0.938742139; 10 | SLE GWAS | 94 | Coronary artery disease
∥ id: ebi-a-GCST005195 | Inverse variance weighted | 82 | −0.031045478 |
0.006861715 | 6.05532E−06; 11 | SLE GWAS | 16 | Coronary artery disease ∥
id: ebi-a-GCST005195 | Inverse variance
weighted | 14 | 0.032458803 | 0.031795388 | 3.07E−01; 12 | SLE GWAS | 56 | Coronary
artery disease ∥ id: ebi-a-GCST005195 | Inverse variance
weighted | 47 | 0.044438749 | 0.026758015 | 0.096760846; 13 | SLE GWAS | 44 | Coronary
artery disease ∥ id: ebi-a-GCST005195 | Inverse variance
weighted | 37 | 0.023726263 | 0.010152644 | 0.019441573; 14 | SLE GWAS | 96 | Coronary
artery disease ∥ id: ebi-a-GCST005195 | Inverse variance
weighted | 81 | 0.01775038 | 0.012810277 | 0.165858095; 15 | SLE GWAS | 6 | Coronary
artery disease ∥ id: ebi-a-GCST005195 | Inverse variance
weighted | 4 | 0.127306876 | 0.049736171 | 0.010477953; 16 | SLE GWAS | 265 | Coronary
artery disease ∥ id: ebi-a-GCST005195 | Inverse variance
weighted 233 | 0.002800088 | 0.003234127 | 0.386603019; 17 | SLE
GWAS | 28 | Coronary artery disease ∥ id: ebi-a-GCST005195 | Inverse variance
weighted | 22 | 0.013437363 | 0.015693935 | 0.391879533; 18 | SLE GWAS | 27 | Coronary
artery disease ∥ id: ebi-a-GCST005195 | Inverse variance
weighted | 25 | 0.05393401 | 0.032951224 | 0.101675776; 19 | SLE GWAS | 216 | Coronary
artery disease ∥ id: ebi-a-GCST005195 | Inverse variance
weighted | 183 | 0.031752113 | 0.006013776 | 1.29257E−07; 20 | SLE
GWAS | 40 | Coronary artery disease ∥ id: ebi-a-GCST005195 | Inverse variance
weighted | 33 | 0.002442942 | 0.006882401 | 0.722623426; 21 | SLE
GWAS | 133 | Coronary artery disease ∥ id: ebi-a-GCST005195 | Inverse variance
weighted | 111 | 0.01224829 | 0.00458308 | 0.007528798; 22 | SLE GWAS | 10 | Coronary
artery disease ∥ id: ebi-a-GCST005195 | Inverse variance
weighted | 8 | 0.034211327 | 0.052173488 | 0.51200273; 23 | SLE GWAS | 14 | Coronary
artery disease ∥ id: ebi-a-GCST005195 | Inverse variance
weighted 13 | 0.049880525 | 0.029285342 | 0.088519592; 24 | SLE GWAS | 27 | Coronary
artery disease ∥ id: ebi-a-GCST005195 | Inverse variance weighted | 20 | −1.71E−01 |
0.012358685 | 9.29433E−44; 25 | SLE GWAS | 51 | Coronary artery disease ∥ id: ebi-a-
GCST005195 | Inverse variance weighted | 42 | −0.056759407 |
0.017421416 | 0.001121906; 27 | SLE GWAS | 28 | Coronary artery disease
∥ id: ebi-a-GCST005195 | Inverse variance
weighted | 19 | 0.025379232 | 0.02034117 | 0.212148985; 28 | SLE GWAS | 27 | Coronary
artery disease ∥ id: ebi-a-GCST005195 | Inverse variance
weighted | 23 | 0.124571316 | 0.046150975 | 0.006950366; 29 | SLE GWAS | 12 | Coronary
artery disease ∥ id: ebi-a-GCST005195 | Inverse variance weighted | 9 | −0.018121477
| 0.045600937 | 0.69107794; 30 | SLE GWAS | 69 | Coronary artery disease ∥
id: ebi-a-GCST005195 | Inverse variance weighted | 59 | −0.081320143
| 0.009080639 | 3.38712E−19; 31 | SLE GWAS | 19 | Coronary artery disease
∥ id: ebi-a-GCST005195 | Inverse variance
weighted | 16 | 0.028651113 | 0.02952178 | 0.331793521; 32 | SLE GWAS | 26 | Coronary
artery disease ∥ id: ebi-a-GCST005195 | Inverse variance
weighted | 22 | 0.014559459 | 0.020377534 | 0.47492683; 33 | SLE GWAS | 41 | Coronary
artery disease ∥ id: ebi-a-GCST005195 | Inverse variance
weighted | 36 | 0.021260752 | 0.007349609 | 0.003818569; 34 | SLE GWAS | 8 | Coronary
artery disease ∥ id: ebi-a-GCST005195 | Inverse variance
weighted | 8 | 0.118806645 | 0.029685927 | 6.27774E−05; 35 | SLE GWAS | 163 | Coronary
artery disease ∥ id: ebi-a-GCST005195 | Inverse variance weighted | 140 | −0.003964601
| 0.0027454 | 0.148714131; 36 | SLE GWAS | 59 | Coronary artery disease ∥
id: ebi-a-GCST005195 | Inverse variance
weighted | 46 | 0.005211352 | 0.008175654 | 0.52384924; 37 | SLE GWAS | 2 | Coronary
artery disease ∥ id: ebi-a-GCST005195 | Inverse variance
weighted | 2 | 0.321811117 | 0.341939117 | 0.346635313; 38 | SLE GWAS | 15 | Coronary
artery disease ∥ id: ebi-a-GCST005195 | Inverse variance
weighted | 14 | 0.093465576 | 0.070978486 | 0.18790045; 39 | SLE GWAS | 12 | Coronary
artery disease ∥ id: ebi-a-GCST005195 | Inverse variance
weighted | 10 | 0.085286529 | 0.061686625 | 0.166794501; 40 | SLE GWAS | 4 | Coronary
artery disease ∥ id: ebi-a-GCST005195 | Inverse variance weighted | 4 | −0.173234218 |
0.075669629 | 0.02205906; 41 | SLE GWAS | 19 | Coronary artery disease ∥
id: ebi-a-GCST005195 | Inverse variance
weighted | 17 | 0.017252731 | 0.038494725 | 0.654020206; 42 | SLE GWAS | 23 | Coronary
artery disease ∥ id: ebi-a-GCST005195 | Inverse variance
weighted | 20 | 0.042548155 | 0.04584911 | 0.353405519; 43 | SLE GWAS | 27 | Coronary
artery disease ∥ id: ebi-a-GCST005195 | Inverse variance
weighted | 18 | 0.01986561 | 0.023387854 | 0.395659547; 44 | SLE GWAS | 36 | Coronary
artery disease ∥ id: ebi-a-GCST005195 | Inverse variance
weighted | 35 | 0.02177331 | 0.005546047 | 8.64008E−05; 45 | SLE GWAS | 77 | Coronary
artery disease ∥ id: ebi-a-GCST005195 | Inverse variance weighted | 71 | −0.027304623
| 0.005371567 | 3.71175E−07; 46 | SLE GWAS | 8 | Coronary artery disease ∥
id: ebi-a-GCST005195 | Inverse variance
weighted | 6 | 0.101846779 | 0.070225389 | 0.146979252; 47 | SLE GWAS | 16 | Coronary
artery disease ∥ id: ebi-a-GCST005195 | Inverse variance weighted | 14 | −0.059959424
| 0.025313512 | 0.017852047; 48 | SLE GWAS | 3 | Coronary artery disease ∥
id: ebi-a-GCST005195 | Inverse variance
weighted | 3 | 0.477754391 | 0.220007366 | 0.029890515; 49 | SLE GWAS | 32 | Coronary
artery disease ∥ id: ebi-a-GCST005195 | Inverse variance weighted | 27 | −0.008025314
| 0.021306555 | 0.706426735; 50 | SLE GWAS | 39 | Coronary artery disease
∥ id: ebi-a-GCST005195 | Inverse variance weighted | 35 | −0.051612716
| 0.007003646 | 1.71389E−13; 51 | SLE GWAS | 5 | Coronary artery disease ∥
id: ebi-a-GCST005195 | Inverse variance
weighted | 4 | 0.113165998 | 0.06320049 | 0.07335954; 52 | SLE GWAS | 8 | Coronary
artery disease ∥ id: ebi-a-GCST005195 | Inverse variance weighted | 8 | −0.020797802
| 0.024770175 | 0.401115422; 53 | SLE GWAS | 9 | Coronary artery disease ∥
id: ebi-a-GCST005195 | Inverse variance
weighted | 7 | 0.008196148 | 0.043163663 | 0.849398958; 54 | SLE GWAS | 4 | Coronary
artery disease ∥ id: ebi-a-GCST005195 | Inverse variance weighted | 4 | −0.048824829
| 0.048676726 | 3.16E−01; 55 | SLE GWAS | 27 | Coronary artery disease ∥
id: ebi-a-GCST005195 | Inverse variance
weighted | 25 | 0.059989799 | 0.02415289 | 0.013000615; 56 | SLE GWAS | 19 | Coronary
artery disease ∥ id: ebi-a-GCST005195 | Inverse variance weighted | 17 | −0.039309193
| 0.023514079 | 0.094577559; 57 | SLE GWAS | 27 | Coronary artery disease
∥ id: ebi-a-GCST005195 | Inverse variance
weighted | 25 | 0.03455255 | 0.026071626 | 0.185073903; 58 | SLE GWAS | 10 | Coronary
artery disease ∥ id: ebi-a-GCST005195 | Inverse variance
weighted | 8 | 0.018773443 | 0.077912236 | 0.809588994; 59 | SLE GWAS | 8 | Coronary
artery disease ∥ id: ebi-a-GCST005195 | Inverse variance
weighted | 8 | 0.058488333 | 0.072962523 | 0.422771896; 60 | SLE GWAS | 4 | Coronary
artery disease ∥ id: ebi-a-GCST005195 | Inverse variance
weighted | 4 | 0.303241428 | 0.119675173 | 0.011281031; 61 | SLE GWAS | 79 | Coronary
artery disease ∥ id: ebi-a-GCST005195 | Inverse variance weighted | 71 | −0.022619067
| 0.00559705 | 5.31675E−05; 62 | SLE GWAS | 7 | Coronary artery disease ∥
id: ebi-a-GCST005195 | Inverse variance weighted | 7 | −0.039048649
| 0.037474145 | 0.297404378; 63 | SLE GWAS | 14 | Coronary artery disease
∥ id: ebi-a-GCST005195 | Inverse variance
weighted | 12 | 0.037756476 | 0.033134225 | 0.254494323; 64 | SLE GWAS | 35 | Coronary
artery disease ∥ id: ebi-a-GCST005195 | Inverse variance weighted | 32 | −0.051005625
| 0.007027391 | 3.92517E−13; 65 | SLE GWAS | 22 | Coronary artery disease
∥ id: ebi-a-GCST005195 | Inverse variance
weighted | 18 | 0.001103922 | 0.014287769 | 0.938413999; 66 | SLE GWAS | 7 | Coronary
artery disease ∥ id: ebi-a-GCST005195 | Inverse variance weighted | 6 | −0.160179053
| 0.093118653 | 0.085403225; 67 | SLE GWAS | 17 | Coronary artery disease
∥ id: ebi-a-GCST005195 | Inverse variance
weighted | 14 | 0.073853823 | 0.039001373 | 0.058275318; 1 | SLE GWAS | 133 | Ischemic
stroke ∥ id: ieu-a-1108 | Inverse variance
weighted | 83 | 0.041718395 | 0.012732658 | 0.001051069; 2 | SLE GWAS | 191 | Ischemic
stroke ∥ id: ieu-a-1108 | Inverse variance
weighted | 146 | 0.025287966 | 0.007905657 | 0.001380403; 3 | SLE GWAS | 62 | Ischemic
stroke ∥ id: ieu-a-1108 | Inverse variance weighted | 40 | −0.024188447
| 0.010962574 | 0.027352028; 4 | SLE GWAS | 95 | Ischemic stroke ∥ id: ieu-
a-1108 | Inverse variance weighted | 70 | 0.019330511 | 0.018595546 | 0.298561249;
5 | SLE GWAS | 143 | Ischemic stroke ∥ id: ieu-a-1108 | Inverse variance
weighted | 97 | 0.036498454 | 0.01034977 | 0.000421093; 6 | SLE GWAS | 61 | Ischemic
stroke ∥ id: ieu-a-1108 | Inverse variance
weighted | 44 | 0.083059317 | 0.013690968 | 1.30546E−09; 7 | SLE GWAS | 56 | Ischemic
stroke ∥ id: ieu-a-1108 | Inverse variance
weighted | 36 | 0.014448916 | 0.022687795 | 0.524216994; 8 | SLE GWAS | 22 | Ischemic
stroke ∥ id: ieu-a-1108 | Inverse variance weighted | 15 | −0.064792692
| 0.039877749 | 0.104208786; 9 | SLE GWAS | 264 | Ischemic stroke ∥ id: ieu-
a-1108 | Inverse variance weighted | 188 | 0.051276574 | 0.006252408 | 2.38212E−16;
10 | SLE GWAS | 94 | Ischemic stroke ∥ id: ieu-a-1108 | Inverse variance weighted | 59 | −0.030144561
| 0.017458703 | 0.084235766; 11 | SLE GWAS | 16 | Ischemic stroke ∥ id: ieu-
a-1108 | Inverse variance weighted | 13 | −0.010372266 | 0.038697153 | 0.788670906;
12 | SLE GWAS | 56 | Ischemic stroke ∥ id: ieu-a-1108 | Inverse variance
weighted | 42 | 0.04258166 | 0.043151527 | 0.323743725; | 13 | SLE GWAS | 44 | Ischemic
stroke ∥ id: ieu-a-1108 | Inverse variance
weighted | 30 | 0.10352942 | 0.024623385 | 2.61641E−05; 14 | SLE GWAS | 96 | Ischemic
stroke ∥ id: ieu-a-1108 | Inverse variance
weighted | 74 | 0.008158487 | 0.021875778 | 0.709188512; 15 | SLE GWAS | 6 | Ischemic
stroke ∥ id: ieu-a-1108 | Inverse variance
weighted | 3 | 0.029008466 | 0.11238495 | 0.796316613; 16 | SLE GWAS | 265 | Ischemic
stroke ∥ id: ieu-a-1108 | Inverse variance
weighted | 194 | 0.056783998 | 0.00745139 | 2.52517E−14; 17 | SLE GWAS | 28 | Ischemic
stroke ∥ id: ieu-a-1108 | Inverse variance weighted | 20 | −0.093173241
| 0.041789046 | 0.025773399; 18 | SLE GWAS | 27 | Ischemic stroke ∥ id: ieu-
a-1108 | Inverse variance weighted | 24 | 0.09532139 | 0.040835809 | 0.019582119;
19 | SLE GWAS | 216 | Ischemic stroke ∥ id: ieu-a-1108 | Inverse variance
weighted | 163 | 0.058678434 | 0.010591528 | 3.02248E−08; 20 | SLE
GWAS | 40 | Ischemic stroke ∥ id: ieu-a-1108 | Inverse variance
weighted | 29 | 0.007773556 | 0.016191973 | 0.63116552; 21 | SLE GWAS | 133 | Ischemic
stroke ∥ id: ieu-a-1108 | Inverse variance
weighted | 97 | 0.038129374 | 0.011591387 | 0.001003807; 22 | SLE GWAS | 10 | Ischemic
stroke ∥ id: ieu-a-1108 | Inverse variance
weighted | 8 | 0.196874399 | 0.127545066 | 0.122693179; 23 | SLE GWAS | 14 | Ischemic
stroke ∥ id: ieu-a-1108 | Inverse variance
weighted | 10 | 0.071242893 | 0.052735515 | 0.176712373; 24 | SLE GWAS | 27 | Ischemic
stroke ∥ id: ieu-a-1108 | Inverse variance
weighted | 19 | 0.150964256 | 0.026825925 | 1.82786E−08; 25 | SLE GWAS | 51 | Ischemic
stroke ∥ id: ieu-a-1108 | Inverse variance
weighted | 35 | 0.087698179 | 0.022880954 | 0.000126692; 27 | SLE GWAS | 28 | Ischemic
stroke ∥ id: ieu-a-1108 | Inverse variance
weighted | 15 | 0.108931697 | 0.057489608 | 0.058118009; 28 | SLE GWAS | 27 | Ischemic
stroke ∥ id: ieu-a-1108 | Inverse variance
weighted | 20 | 0.11990551 | 0.090352047 | 0.184478134; 29 | SLE GWAS | 12 | Ischemic
stroke ∥ id: ieu-a-1108 | Inverse variance
weighted | 9 | 0.027468676 | 0.061784548 | 0.656617428; 30 | SLE GWAS | 69 | Ischemic
stroke ∥ id: ieu-a-1108 | Inverse variance
weighted | 35 | 0.017787687 | 0.027325154 | 0.515069827; 31 | SLE GWAS | 19 | Ischemic
stroke ∥ id: ieu-a-1108 | Inverse variance weighted | 14 | −0.001554397
| 0.056640769 | 0.978106347; 32 | SLE GWAS | 26 | Ischemic stroke ∥ id: ieu-
a-1108 | Inverse variance weighted | 16 | 0.039669349 | 0.032345983 | 0.220045458;
33 | SLE GWAS | 41 | Ischemic stroke ∥ id: ieu-a-1108 | Inverse variance
weighted | 26 | 0.059031413 | 0.017968276 | 0.001018691; 34 | SLE GWAS | 8 | Ischemic
stroke ∥ id: ieu-a-1108 | Inverse variance
weighted | 6 | 0.102890972 | 0.047268849 | 0.029501586; 35 | SLE GWAS | 163 | Ischemic
stroke ∥ id: ieu-a-1108 | Inverse variance
weighted | 115 | 0.047411741 | 0.007166811 | 3.70402E−11; 36 | SLE
GWAS | 59 | Ischemic stroke ∥ id: ieu-a-1108 | Inverse variance weighted | 45 | −0.003298769
| 0.019518161 | 0.86578859; 38 | SLE GWAS | 15 | Ischemic stroke ∥ id: ieu-
a-1108 | Inverse variance weighted 13 | 0.092342342 | 0.11245832 | 0.41157505; 39 | SLE
GWAS | 12 | Ischemic stroke ∥ id: ieu-a-1108 | Inverse variance
weighted | 8 | 0.110808914 | 0.111945466 | 0.322248775; 40 | SLE GWAS | 4 | Ischemic
stroke ∥ id: ieu-a-1108 | Inverse variance
weighted | 4 | 0.173950899 | 0.179631777 | 0.332857186; 41 | SLE GWAS | 19 | Ischemic
stroke ∥ id: ieu-a-1108 | Inverse variance
weighted | 17 | 0.011092777 | 0.075689737 | 0.883482574; 42 | SLE GWAS | 23 | Ischemic
stroke ∥ id: ieu-a-1108 | Inverse variance
weighted | 17 | 0.02911477 | 0.057274564 | 0.611217153; 43 | SLE GWAS | 27 | Ischemic
stroke ∥ id: ieu-a-1108 | Inverse variance
weighted | 13 | 0.026129078 | 0.045849268 | 0.568751734; 44 | SLE GWAS | 36 | Ischemic
stroke ∥ id: ieu-a-1108 | Inverse variance
weighted | 28 | 0.050977896 | 0.014188104 | 0.000326889; 45 | SLE GWAS | 77 | Ischemic
stroke ∥ id: ieu-a-1108 | Inverse variance weighted | 42 | −0.101744068
| 0.016892479 | 1.71172E−09; 46 | SLE GWAS | 8 | Ischemic stroke ∥ id: ieu-
a-1108 | Inverse variance weighted | 5 | 0.047086369 | 0.079537708 | 0.553850204;
47 | SLE GWAS | 16 | Ischemic stroke ∥ id: ieu-a-1108 | Inverse variance weighted | 13
| −0.016124575 | 0.059315771 | 0.785742773; 48 | SLE GWAS | 3 | Ischemic stroke ∥ id: ieu-
a-1108 | Inverse variance weighted | 3 | 0.52391788 | 0.2841727 | 0.065232685; 49 | SLE
GWAS | 32 | Ischemic stroke ∥ id: ieu-a-1108 | Inverse variance weighted | 23 | −0.034502696
| 0.044090771 | 0.433898569; 50 | SLE GWAS | 39 | Ischemic stroke ∥ id: ieu-
a-1108 | Inverse variance weighted | 14 | −0.107899022 | 0.028428135 | 0.000147345;
51 | SLE GWAS | 5 | Ischemic stroke ∥ id: ieu-a-1108 | Inverse variance
weighted | 3 | 0.030351471 | 0.145191882 | 0.834414004; 52 | SLE GWAS | 8 | Ischemic
stroke ∥ id: ieu-a-1108 | Inverse variance weighted | 8 | −0.0328968
| 0.056806955 | 0.562522976; 53 | SLE GWAS | 9 | Ischemic stroke ∥ id: ieu-a-
1108 | Inverse variance weighted | 7 | −0.201530005 | 0.090151337 | 0.025386949; 54 | SLE
GWAS | 4 | Ischemic stroke ∥ id: ieu-a-1108 | Inverse variance weighted | 4 | −0.036277235
| 0.116052597 | 0.754589861; 55 | SLE GWAS | 27 | Ischemic stroke ∥ id: ieu-
a-1108 | Inverse variance weighted | 23 | 0.041379735 | 0.038728595 | 0.285315617;
56 | SLE GWAS | 19 | Ischemic stroke ∥ id: ieu-a-1108 | Inverse variance weighted | 16
| −0.044449471 | 0.048721811 | 0.361604729; 57 | SLE GWAS | 27 | Ischemic stroke ∥ id: ieu-
a-1108 | Inverse variance weighted | 21 | 0.040455154 | 0.039670126 | 0.307828594;
58 | SLE GWAS | 10 | Ischemic stroke ∥ id: ieu-a-1108 | Inverse variance
weighted | 8 | 0.222399891 | 0.10397417 | 0.032436337; 59 | SLE GWAS | 8 | Ischemic
stroke ∥ id: ieu-a-1108 | Inverse variance
weighted | 8 | 0.034403322 0.121949789 | 0.777858743; 60 | SLE GWAS | 4 | Ischemic
stroke ∥ id: ieu-a-1108 | Inverse variance weighted | 4 | −0.07766227
| 0.169364603 | 0.646556793; 61 | SLE GWAS | 79 | Ischemic stroke ∥ id: ieu-
a-1108 | Inverse variance weighted | 68 | 0.057478646 | 0.013293346 | 1.53318E−05;
62 | SLE GWAS | 7 | Ischemic stroke ∥ id: ieu-a-1108 | Inverse variance weighted | 7
| −0.113564616 | 0.067877163 | 0.094309544; 63 | SLE GWAS | 14 | Ischemic stroke ∥ id: ieu-
a-1108 | Inverse variance weighted | 11 | 0.071202057 | 0.049418162 | 0.14963908;
64 | SLE GWAS | 35 | Ischemic stroke ∥ id: ieu-a-1108 | Inverse variance weighted | 13
| −0.097560537 | 0.027616807 | 0.000411415; 65 | SLE GWAS | 22 | Ischemic stroke ∥ id: ieu-
a-1108 | Inverse variance weighted | 17 | 0.052649183 | 0.033116069 | 0.111871342;
66 | SLE GWAS | 7 | Ischemic stroke ∥ id: ieu-a-1108 | Inverse variance
weighted | 5 | 0.045169643 | 0.224777692 | 0.840735703; 67 | SLE GWAS | 17 | Ischemic
stroke ∥ id: ieu-a-1108 | Inverse variance
weighted | 11 | 0.063049006 | 0.068023993 | 0.353997029; 1 | SLE
GWAS | 133 | Myocardial infarction ∥ id: ebi-a-GCST011365 | Inverse variance
weighted | 115 | −0.005330484 | 0.008277429 | 0.519589551; 2 | SLE
GWAS | 191 | Myocardial infarction ∥ id: ebi-a-GCST011365 | Inverse variance
weighted | 168 | 0.024025023 | 0.003360251 | 8.69235E−13; 3 | SLE
GWAS | 62 | Myocardial infarction ∥ id: ebi-a-GCST011365 | Inverse variance
weighted | 46 0.018897949 | 0.005682103 | 0.000881423; 4 | SLE
GWAS | 95 | Myocardial infarction ∥ id: ebi-a-GCST011365 | Inverse variance
weighted 84 | 0.051739198 | 0.009342841 | 3.06218E−08; 5 | SLE
GWAS | 143 | Myocardial infarction ∥ id: ebi-a-GCST011365 | Inverse variance
weighted | 124 | 0.022823971 | 0.005531838 | 3.69241E−05; 6 | SLE
GWAS | 61 | Myocardial infarction ∥ id: ebi-a-GCST011365 | Inverse variance
weighted | 51 | 0.006701316 | 0.014330597 | 0.640054259; 7 | SLE
GWAS | 56 | Myocardial infarction ∥ id: ebi-a-GCST011365 | Inverse variance
weighted | 50 | −0.011880956 | 0.010712133 | 0.267381826; 8 | SLE
GWAS | 22 | Myocardial infarction ∥ id: ebi-a-GCST011365 | Inverse variance
weighted | 18 | 0.069652939 | 0.013110274 | 1.07923E−07; 9 | SLE
GWAS | 264 | Myocardial infarction ∥ id: ebi-a-GCST011365 | Inverse variance
weighted | 231 | −0.002460339 | 0.003491455 | 0.481012894; 10 | SLE
GWAS | 94 | Myocardial infarction ∥ id: ebi-a-GCST011365 | Inverse variance
weighted | 82 | −0.024110452 | 0.007790955 | 0.001970304; 11 | SLE
GWAS | 16 | Myocardial infarction ∥ id: ebi-a-GCST011365 | Inverse variance
weighted | 14 | 0.003269942 | 0.030719524 | 0.915229245; 12 | SLE
GWAS | 56 | Myocardial infarction ∥ id: ebi-a-GCST011365 | Inverse variance
weighted | 47 | 0.062044281 | 0.029712546 | 0.036784221; 13 | SLE
GWAS | 44 | Myocardial infarction ∥ id: ebi-a-GCST011365 | Inverse variance
weighted | 37 | 0.069807457 | 0.011629098 | 1.93912E−09; 14 | SLE
GWAS | 96 | Myocardial infarction ∥ id: ebi-a-GCST011365 | Inverse variance
weighted | 81 | 0.033462589 | 0.011801749 | 0.004576943; 15 | SLE
GWAS | 6 | Myocardial infarction ∥ id: ebi-a-GCST011365 | Inverse variance
weighted | 4 | 0.036364496 | 0.051445447 | 0.479656285; 16 | SLE
GWAS | 265 | Myocardial infarction ∥ id: ebi-a-GCST011365 | Inverse variance
weighted | 233 | 0.007324722 | 0.003187083 | 0.0215474; 17 | SLE
GWAS | 28 | Myocardial infarction ∥ id: ebi-a-GCST011365 | Inverse variance
weighted | 23 | 0.018741114 | 0.016336056 | 0.251289086; 18 | SLE
GWAS | 27 | Myocardial infarction ∥ id: ebi-a-GCST011365 | Inverse variance
weighted | 25 | 0.073532424 | 0.044423335 | 0.097870609; 19 | SLE
GWAS | 216 | Myocardial infarction ∥ id: ebi-a-GCST011365 | Inverse variance
weighted | 183 | 0.060558013 | 0.006825722 | 7.18245E−19; 20 | SLE
GWAS | 40 | Myocardial infarction ∥ id: ebi-a-GCST011365 | Inverse variance
weighted | 34 | 0.008651933 | 0.00697851 | 0.215050654; 21 | SLE
GWAS | 133 | Myocardial infarction ∥ id: ebi-a-GCST011365 | Inverse variance
weighted | 111 | 0.030168776 | 0.004992634 | 1.51597E−09; 22 | SLE
GWAS | 10 | Myocardial infarction ∥ id: ebi-a-GCST011365 | Inverse variance
weighted | 8 | 0.066476404 | 0.059607536 | 0.264749785; 23 | SLE
GWAS | 14 | Myocardial infarction ∥ id: ebi-a-GCST011365 | Inverse variance
weighted | 13 | 0.066344893 | 0.034865144 | 0.057053554; 24 | SLE
GWAS | 27 | Myocardial infarction ∥ id: ebi-a-GCST011365 | Inverse variance
weighted | 20 | −0.184687361 | 0.01363486 | 8.45443E−42; 25 | SLE
GWAS | 51 | Myocardial infarction ∥ id: ebi-a-GCST011365 | Inverse variance
weighted | 42 | −0.066468636 | 0.019321982 | 0.000581601; 27 | SLE
GWAS | 28 | Myocardial infarction ∥ id: ebi-a-GCST011365 | Inverse variance
weighted | 19 | 0.020399311 | 0.032257285 | 0.527129501; 28 | SLE
GWAS | 27 | Myocardial infarction ∥ id: ebi-a-GCST011365 | Inverse variance
weighted | 23 | 0.173672616 | 0.051657142 | 0.00077373; 29 | SLE
GWAS | 12 | Myocardial infarction ∥ id: ebi-a-GCST011365 | Inverse variance
weighted | 9 | −0.035762219 | 0.058495938 | 0.540959666; 30 | SLE
GWAS | 69 | Myocardial infarction ∥ id: ebi-a-GCST011365 | Inverse variance
weighted | 59 | −0.079493578 | 0.010301634 | 1.19475E−14; 31 | SLE
GWAS | 19 | Myocardial infarction ∥ id: ebi-a-GCST011365 | Inverse variance
weighted | 16 | 0.067821595 | 0.036684188 | 0.064487218; 32 | SLE
GWAS | 26 | Myocardial infarction ∥ id: ebi-a-GCST011365 | Inverse variance
weighted | 22 | 0.020247512 | 0.016383659 | 0.216519614; 33 | SLE
GWAS | 41 | Myocardial infarction ∥ id: ebi-a-GCST011365 | Inverse variance
weighted | 36 | 0.055454704 | 0.009872978 | 1.94508E−08; 34 | SLE
GWAS | 8 | Myocardial infarction ∥ id: ebi-a-GCST011365 | Inverse variance
weighted | 8 | 0.166683393 | 0.029911176 | 2.50947E−08; 35 | SLE
GWAS | 163 | Myocardial infarction ∥ id: ebi-a-GCST011365 | Inverse variance
weighted | 140 | −0.018821589 | 0.003061022 | 7.80755E−10; 36 | SLE
GWAS | 59 | Myocardial infarction ∥ id: ebi-a-GCST011365 | Inverse variance
weighted | 46 | 0.009377451 | 0.00900556 | 0.297738354; 37 | SLE GWAS | 2 | Myocardial
infarction ∥ id: ebi-a-GCST011365 | Inverse variance
weighted | 2 | 0.185966596 | 0.288634616 | 0.519382459; 38 | SLE
GWAS | 15 | Myocardial infarction ∥ id: ebi-a-GCST011365 | Inverse variance
weighted | 14 | 0.097927252 | 0.074171701 | 0.186742279; 39 | SLE
GWAS | 12 | Myocardial infarction ∥ id: ebi-a-GCST011365 | Inverse variance
weighted | 10 | 0.158522347 | 0.060494266 | 0.008781317; 40 | SLE
GWAS | 4 | Myocardial infarction ∥ id: ebi-a-GCST011365 | Inverse variance
weighted | 4 | −0.188778527 | 0.083058913 | 0.023036617; 41 | SLE
GWAS | 19 | Myocardial infarction ∥ id: ebi-a-GCST011365 | Inverse variance
weighted | 17 | 0.065678768 | 0.03728339 | 0.078135323; 42 | SLE
GWAS | 23 | Myocardial infarction ∥ id: ebi-a-GCST011365 | Inverse variance
weighted | 20 | 0.034291869 | 0.038619832 | 0.374576155; 43 | SLE
GWAS | 27 | Myocardial infarction ∥ id: ebi-a-GCST011365 | Inverse variance
weighted | 18 | 0.026038042 | 0.025040574 | 0.298417009; 44 | SLE
GWAS | 36 | Myocardial infarction ∥ id: ebi-a-GCST011365 | Inverse variance
weighted | 35 | 0.048205122 | 0.006451736 | 7.91947E−14; 45 | SLE
GWAS | 77 | Myocardial infarction ∥ id: ebi-a-GCST011365 | Inverse variance
weighted | 71 | −0.03473608 | 0.005941065 | 5.0119E−09; 46 | SLE GWAS | 8 | Myocardial
infarction ∥ id: ebi-a-GCST011365 | Inverse variance
weighted | 6 | 0.087833966 | 0.079891805 | 0.271588638; 47 | SLE
GWAS | 16 | Myocardial infarction ∥ id: ebi-a-GCST011365 | Inverse variance
weighted | 14 | −0.017934269 | 0.036853657 | 0.626517208; 48 | SLE
GWAS | 3 | Myocardial infarction ∥ id: ebi-a-GCST011365 | Inverse variance
weighted | 3 | 0.519291596 | 0.252579162 | 0.039786776; 49 | SLE
GWAS | 32 | Myocardial infarction ∥ id: ebi-a-GCST011365 | Inverse variance
weighted | 27 | −0.007011802 | 0.023774838 | 0.768050909; 50 | SLE
GWAS | 39 | Myocardial infarction ∥ id: ebi-a-GCST011365 | Inverse variance
weighted | 35 | −0.049086106 | 0.007931486 | 6.06371E−10; 51 | SLE
GWAS | 5 | Myocardial infarction ∥ id: ebi-a-GCST011365 | Inverse variance
weighted | 4 | 0.101084493 | 0.05642051 | 0.073192781; 52 | SLE GWAS | 8 | Myocardial
infarction ∥ id: ebi-a-GCST011365 | Inverse variance
weighted | 8 | 0.02150451 | 0.031995503 | 0.501513432; 53 | SLE GWAS | 9 | Myocardial
infarction ∥ id: ebi-a-GCST011365 | Inverse variance
weighted | 7 | 0.017327604 | 0.046047528 | 0.706695177; 54 | SLE GWAS | 4 | Myocardial
infarction ∥ id: ebi-a-GCST011365 | Inverse variance weighted | 4 | −0.000544348
| 0.054701232 | 0.99206015; 55 | SLE GWAS | 27 | Myocardial infarction ∥
id: ebi-a-GCST011365 | Inverse variance
weighted | 25 | 0.025350647 | 0.018626027 | 0.173503098; 56 | SLE
GWAS | 19 | Myocardial infarction ∥ id: ebi-a-GCST011365 | Inverse variance
weighted | 17 | −0.042232599 | 0.025246671 | 0.094366926; 57 | SLE
GWAS | 27 | Myocardial infarction ∥ id: ebi-a-GCST011365 | Inverse variance
weighted | 25 | 0.041776993 | 0.026281071 | 0.111919828; 58 | SLE
GWAS | 10 | Myocardial infarction ∥ id: ebi-a-GCST011365 | Inverse variance
weighted | 8 | 0.059768175 | 0.073329544 | 0.415036431; 59 | SLE GWAS | 8 | Myocardial
infarction ∥ id: ebi-a-GCST011365 | Inverse variance
weighted | 8 | 0.129912695 | 0.073850751 | 7.86E−02; 60 | SLE GWAS | 4 | Myocardial
infarction ∥ id: ebi-a-GCST011365 | Inverse variance
weighted | 4 | 0.151742843 | 0.077408272 | 0.049961631; 61 | SLE
GWAS | 79 | Myocardial infarction ∥ id: ebi-a-GCST011365 | Inverse variance
weighted | 71 | 0.011322792 | 0.005887467 | 0.054454595; 62 | SLE
GWAS | 7 | Myocardial infarction ∥ id: ebi-a-GCST011365 | Inverse variance
weighted | 7 | −0.015044603 | 0.048699232 | 0.757375572; 63 | SLE
GWAS | 14 | Myocardial infarction ∥ id: ebi-a-GCST011365 | Inverse variance
weighted | 12 | 0.004006484 | 0.040524207 | 0.921244325; 64 | SLE
GWAS | 35 | Myocardial infarction ∥ id: ebi-a-GCST011365 | Inverse variance
weighted | 32 | −0.050172082 | 0.007977041 | 3.18367E−10; 65 | SLE
GWAS | 22 | Myocardial infarction ∥ id: ebi-a-GCST011365 | Inverse variance
weighted | 18 | 0.088049178 | 0.014763298 | 2.46048E−09; 66 | SLE
GWAS | 7 | Myocardial infarction ∥ id: ebi-a-GCST011365 | Inverse variance
weighted | 6 | −0.221302815 | 0.072586697 | 0.002297522; 67 | SLE
GWAS | 17 | Myocardial infarction ∥ id: ebi-a-GCST011365 | Inverse variance
weighted | 14 | 0.093315824 | 0.044094386 | 0.03432147; 1 | SLE
GWAS | 133 | Myocardial infarction ∥ id: ieu-a-798 | Inverse variance weighted | 114
| −0.007017319 | 0.007949999 | 0.377408252; 2 | SLE GWAS | 191 | Myocardial infarction ∥
id: ieu-a-798 | Inverse variance weighted | 168 | 0.021509797 | 0.004536331 | 2.11961E−06;
3 | SLE GWAS | 62 | Myocardial infarction ∥ id: ieu-a-798 | Inverse variance
weighted | 46 | −0.003654885 | 0.007526359 | 0.627242804; 4 | SLE
GWAS | 95 | Myocardial infarction ∥ id: ieu-a-798 | Inverse variance
weighted | 84 | 0.048767989 | 0.011379565 | 1.82266E−05; 5 | SLE
GWAS | 143 | Myocardial infarction ∥ id: ieu-a-798 | Inverse variance
weighted | 124 | 0.01488386 | 0.006755209 | 0.027572489; 6 | SLE
GWAS | 61 | Myocardial infarction ∥ id: ieu-a-798 | Inverse variance
weighted | 51 | 0.022980793 | 0.008502953 | 0.006878212; 7 | SLE
GWAS | 56 | Myocardial infarction ∥ id: ieu-a-798 | Inverse variance weighted | 49
| −0.020864061 | 0.012562537 | 0.096750432; 8 | SLE GWAS | 22 | Myocardial infarction ∥
id: ieu-a-798 | Inverse variance weighted | 18 | 0.087250517 | 0.018639897 | 2.85691E−06;
9 | SLE GWAS | 264 | Myocardial infarction ∥ id: ieu-a-798 | Inverse variance
weighted | 231 | 0.004622029 | 0.004004958 | 0.248468659; 10 | SLE
GWAS | 94 | Myocardial infarction ∥ id: ieu-a-798 | Inverse variance weighted | 82
| −0.031462112 | 0.009829405 | 0.001370393; 11 | SLE GWAS | 16 | Myocardial infarction ∥
id: ieu-a-798 | Inverse variance weighted | 14 | −0.027898066 | 0.029463736 | 0.343709562;
12 | SLE GWAS | 56 | Myocardial infarction ∥ id: ieu-a-798 | Inverse variance
weighted | 47 | 0.057733706 | 0.030656977 | 0.059671121; 13 | SLE
GWAS | 44 | Myocardial infarction ∥ id: ieu-a-798 | Inverse variance
weighted | 36 | 0.061201764 | 0.013321204 | 4.3418E−06; 14 | SLE
GWAS | 96 | Myocardial infarction ∥ id: ieu-a-798 | Inverse variance
weighted | 81 | 0.039926083 | 0.014840022 | 0.00713594; 15 | SLE GWAS | 6 | Myocardial
infarction ∥ id: ieu-a-798 | Inverse variance weighted | 4
| −0.002607206 | 0.069286805 | 0.969983335; 16 | SLE GWAS | 265 | Myocardial infarction
∥ id: ieu-a-798 | Inverse variance weighted | 233 | −0.000589579
| 0.004299673 | 0.890934448; 17 | SLE GWAS | 28 | Myocardial infarction ∥
id: ieu-a-798 | Inverse variance weighted | 23 | 0.025974634 | 0.022014912 | 0.238053742;
18 | SLE GWAS | 27 | Myocardial infarction ∥ id: ieu-a-798 | Inverse variance
weighted | 25 | 0.110753448 | 0.048660002 | 0.02284197; 19 | SLE
GWAS | 216 | Myocardial infarction ∥ id: ieu-a-798 | Inverse variance
weighted | 183 | 0.052866706 | 0.007549764 | 2.51558E−12; 20 | SLE
GWAS | 40 | Myocardial infarction ∥ id: ieu-a-798 | Inverse variance
weighted | 35 | 0.004796611 | 0.009147018 | 0.600006795; 21 | SLE
GWAS | 133 | Myocardial infarction ∥ id: ieu-a-798 | Inverse variance
weighted | 111 | 0.027862735 | 0.007052881 | 7.79729E−05; 22 | SLE
GWAS | 10 | Myocardial infarction ∥ id: ieu-a-798 | Inverse variance
weighted | 8 | 0.116773644 | 0.08038166 | 0.146295989; 23 | SLE GWAS | 14 | Myocardial
infarction ∥ id: ieu-a-798 | Inverse variance
weighted | 13 | 0.109415764 | 0.038857365 | 0.004865128; 24 | SLE
GWAS | 27 | Myocardial infarction ∥ id: ieu-a-798 | Inverse variance weighted | 20
| −0.040513365 | 0.019429317 | 0.037054171; 25 | SLE GWAS | 51 | Myocardial infarction ∥
id: ieu-a-798 | Inverse variance weighted | 42 | 0.002135317 | 0.015460553 | 0.890150419;
27 | SLE GWAS | 28 | Myocardial infarction ∥ id: ieu-a-798 | Inverse variance
weighted | 19 | 0.031490108 | 0.03062529 | 0.303837563; 28 | SLE
GWAS | 27 | Myocardial infarction ∥ id: ieu-a-798 | Inverse variance
weighted | 23 | 0.167832346 | 0.054221673 | 0.001966139; 29 | SLE
GWAS | 12 | Myocardial infarction ∥ id: ieu-a-798 | Inverse variance weighted | 9
| −0.082037375 | 0.068460184 | 0.230791512; 30 | SLE GWAS | 69 | Myocardial infarction ∥
id: ieu-a-798 | Inverse variance weighted | 59 | −0.052840621 | 0.009934776 | 1.04481E−07;
31 | SLE GWAS | 19 | Myocardial infarction ∥ id: ieu-a-798 | Inverse variance
weighted | 16 | 0.066273551 | 0.047672734 | 0.16447505; 32 | SLE
GWAS | 26 | Myocardial infarction ∥ id: ieu-a-798 | Inverse variance
weighted | 22 | 0.030027897 | 0.018620735 | 0.106830279; 33 | SLE
GWAS | 41 | Myocardial infarction ∥ id: ieu-a-798 | Inverse variance
weighted | 36 | 0.043408718 | 0.011810197 | 0.000237357; 34 | SLE
GWAS | 8 | Myocardial infarction ∥ id: ieu-a-798 | Inverse variance
weighted | 8 | 0.130993628 | 0.028266798 | 3.58343E−06; 35 | SLE
GWAS | 163 | Myocardial infarction ∥ id: ieu-a-798 | Inverse variance weighted | 140
| −0.010609565 | 0.004390138 | 0.015662726; 36 | SLE GWAS | 59 | Myocardial infarction ∥
id: ieu-a-798 | Inverse variance weighted | 46 | 0.002513662 | 0.012877345 | 0.845236152;
37 | SLE GWAS | 2 | Myocardial infarction ∥ id: ieu-a-798 | Inverse variance
weighted | 2 | 0.04010741 | 0.262841424 | 0.878720299; 38 | SLE GWAS | 15 | Myocardial
infarction ∥ id: ieu-a-798 | Inverse variance
weighted | 14 | 0.081459261 | 0.07881423 | 0.301341762; 39 | SLE
GWAS | 12 | Myocardial infarction ∥ id: ieu-a-798 | Inverse variance
weighted | 10 | 0.152233466 | 0.057961491 | 0.008627845; 40 | SLE
GWAS | 4 | Myocardial infarction ∥ id: ieu-a-798 | Inverse variance weighted | 4
| −0.246917607 | 0.118179592 | 0.036676932; 41 | SLE GWAS | 19 | Myocardial infarction ∥
id: ieu-a-798 | Inverse variance weighted | 18 | 0.132130567 | 0.047945819 | 5.85E−03;
42 | SLE GWAS | 23 | Myocardial infarction ∥ id: ieu-a-798 | Inverse variance
weighted | 21 | 0.027677919 | 0.036944944 | 0.453756784; 43 | SLE
GWAS | 27 | Myocardial infarction ∥ id: ieu-a-798 | Inverse variance
weighted | 18 | 0.034537271 0.028005275 | 0.217485622; 44 | SLE
GWAS | 36 | Myocardial infarction ∥ id: ieu-a-798 | Inverse variance
weighted | 35 | 0.034951201 | 0.008452649 | 3.55047E−05; 45 | SLE
GWAS | 77 | Myocardial infarction ∥ id: ieu-a-798 | Inverse variance weighted | 71
| −0.047102006 | 0.00851459 | 3.1675E−08; 46 | SLE GWAS | 8 | Myocardial infarction ∥
id: ieu-a-798 | Inverse variance weighted | 6 | 0.103816847 | 0.101674116 | 0.307219144;
47 | SLE GWAS | 16 | Myocardial infarction ∥ id: ieu-a-798 | Inverse variance
weighted | 13 | 0.017225885 | 0.049362094 | 0.727111913; 48 | SLE
GWAS | 3 | Myocardial infarction ∥ id: ieu-a-798 | Inverse variance
weighted | 3 | 0.510500138 | 0.27226624 | 0.060792249; 49 | SLE GWAS | 32 | Myocardial
infarction ∥ id: ieu-a-798 | Inverse variance weighted | 26 | −0.006476143
| 0.02762553 | 8.15E−01; 50 | SLE GWAS | 39 | Myocardial infarction ∥
id: ieu-a-798 | Inverse variance weighted | 35 | −0.063939466 | 0.011686514 | 4.46953E−08;
51 | SLE GWAS | 5 | Myocardial infarction ∥ id: ieu-a-798 | Inverse variance
weighted | 4 | 0.020496806 | 0.077973373 | 0.792651317; 52 | SLE GWAS | 8 | Myocardial
infarction ∥ id: ieu-a-798 | Inverse variance
weighted | 8 | 0.066508678 | 0.03566938 | 0.062239259; 53 | SLE GWAS | 9 | Myocardial
infarction ∥ id: ieu-a-798 | Inverse variance
weighted | 7 | 0.047246509 | 0.049177135 | 0.336682222; 54 | SLE GWAS | 4 | Myocardial
infarction ∥ id: ieu-a-798 | Inverse variance
weighted | 4 | 0.072707706 | 0.095982649 | 0.448745064; 55 | SLE
GWAS | 27 | Myocardial infarction ∥ id: ieu-a-798 | Inverse variance
weighted | 25 | 0.010771725 | 0.023524541 | 0.647029235; 56 | SLE
GWAS | 19 | Myocardial infarction ∥ id: ieu-a-798 | Inverse variance weighted | 17
| −0.026003783 | 0.036165819 | 0.472131388; 57 | SLE GWAS | 27 | Myocardial infarction ∥
id: ieu-a-798 | Inverse variance weighted | 25 | 0.010564075 | 0.027145014 | 6.97E−01;
58 | SLE GWAS | 10 | Myocardial infarction ∥ id: ieu-a-798 | Inverse variance
weighted | 8 | 0.091933795 | 0.100473136 | 0.360187054; 59 | SLE GWAS | 8 | Myocardial
infarction ∥ id: ieu-a-798 | Inverse variance
weighted | 8 | 0.088493963 | 0.096095668 | 0.357105559; 60 | SLE GWAS | 4 | Myocardial
infarction ∥ id: ieu-a-798 | Inverse variance
weighted | 4 | 0.06301361 | 0.13560919 | 0.64216733; 61 | SLE GWAS | 79 | Myocardial
infarction ∥ id: ieu-a-798 | Inverse variance weighted | 71 | −0.002593656
| 0.008042851 | 0.747089359; 62 | SLE GWAS | 7 | Myocardial infarction ∥
id: ieu-a-798 | Inverse variance weighted | 6 | 0.016833307 | 0.060218678 | 0.779833271;
63 | SLE GWAS | 14 | Myocardial infarction ∥ id: ieu-a-798 | Inverse variance
weighted | 12 | −0.049399137 | 0.037507852 | 0.187827044; 64 | SLE
GWAS | 35 | Myocardial infarction ∥ id: ieu-a-798 | Inverse variance weighted | 32
| −0.061520848 | 0.0117927 | 1.81983E−07; 65 | SLE GWAS | 22 | Myocardial infarction ∥
id: ieu-a-798 | Inverse variance weighted | 18 | 0.125491215 | 0.019892286 | 2.81686E−10;
66 | SLE GWAS | 7 | Myocardial infarction ∥ id: ieu-a-798 | Inverse variance weighted | 6
| −0.209358994 | 0.085732341 | 0.014605855; 67 | SLE GWAS | 17 | Myocardial infarction
id: ieu-a-798 | Inverse variance weighted | 14 | 0.083976434 | 0.043615388 | 0.05418115;

Table 8: Pathway analysis of positive and negative-casual Tier 1 and Tier 2 SLE-derived SNP-predicted protein clusters with significant (p-value<0.05) causal estimates on CAD by PPI-based MR for the comprehensive 67-cluster network.

TABLE 8A
TIER 1 (14/16 MR methods significant): Positive
causal estimates pathway analysis by cluster.

| | | P- | | P- | |
|---|---|---|---|---|---|
| Cluster | IPA | value | EnrichR | value | Dataset |
| 2 | Glucocorticoid | 6.31E−14 | Neutrophil | 3.53E−08 | GO Biological |
| | Receptor Signaling | | degranulation | | Process 2021 |
| | Clathrin-mediated | 5.01E−13 | Fc-gamma receptor | 8.48E−07 | GO Biological |
| | Endocytosis | | signaling pathway | | Process 2021 |
| | Signaling | | involved in | | |
| | | | phagocytosis | | |
| | Actin Nucleation | 2.51E−12 | Neutrophil | 6.06E−06 | Elsevier Pathway |
| | by ARP-WASP | | Activation via | | Collection |
| | Complex | | Adherence on | | |
| | | | Endothelial Cells | | |
| | Regulation of | 6.92E−10 | Neutrophil | 1.08E−05 | Elsevier Pathway |
| | Actin-based | | Degranulation via | | Collection |
| | Motility by Rho | | FPR1/IL8 | | |
| | Integrin Signaling | 9.77E−09 | Leukocyte | 1.15E−05 | Elsevier Pathway |
| | | | Adhesion to | | Collection |
| | | | Endothelial Cell | | |
| 3 | Th1 and Th2 | 1.62E−04 | interleukin-21- | 1.91E−08 | GO Biological |
| | Activation Pathway | | mediated signaling | | Process 2021 |
| | | | pathway | | |
| | Th17 Activation | 2.00E−04 | AHR Signaling in | 1.23E−06 | Elsevier Pathway |
| | Pathway | | Tr1 Cells Function | | Collection |
| | T Cell Exhaustion | 3.63E−04 | JAK-STAT | 5.28E−06 | KEGG 2021 |
| | Signaling Pathway | | signaling pathway | | Human |
| | IL-23 Signaling | 3.80E−04 | Interleukin-6 | 1.89E−05 | BioPlanet 2019 |
| | Pathway | | signaling pathway | | |
| | Role of JAK1 and | 8.71E−04 | Lymphocyte | 1.97E−05 | Elsevier Pathway |
| | JAK3 in yc | | Mediated | | Collection |
| | Cytokine Signaling | | Myocardial Injury | | |
| | | | in Myocarditis | | |
| 4 | EIF2 Signaling | 5.01E−11 | Intracellular | 2.14E−24 | Jensen |
| | | | ribonucleoprotein | | COMPARTMENT |
| | | | complex | | S |
| | Spliceosomal | 2.95E−08 | nuclear-transcribed | 2.82E−23 | GO Biological |
| | Cycle | | mRNA catabolic | | Process 2021 |
| | | | process, nonsense- | | |
| | | | mediated decay | | |
| | Regulation of eIF4 | 5.25E−06 | gene expression | 1.98E−20 | GO Biological |
| | and p70S6K | | | | Process 2021 |
| | Signaling | | | | |
| | Coronavirus | 1.20E−05 | mRNA splicing, via | 7.40E−19 | GO Biological |
| | Pathogenesis | | spliceosome | | Process 2021 |
| | Pathway | | | | |
| | mTOR Signaling | 1.58E−05 | RNA binding | 9.49E−17 | GO Molecular |
| | | | | | Function 2021 |
| 5 | Coagulation | 4.37E−06 | cellular protein | 4.58E−09 | GO Biological |
| | System | | metabolic process | | Process 2021 |
| | Extrinsic | 1.62E−05 | Ischemic stroke | 2.39E−07 | DisGeNET |
| | Prothrombin | | | | |
| | Activation Pathway | | | | |
| | Intrinsic | 3.09E−04 | Lupus | 2.90E−06 | DisGeNET |
| | Prothrombin | | Erythematosus, | | |
| | Activation Pathway | | Systemic | | |
| | dsDNA Break | 8.71E−04 | low-density | 8.50E−05 | GO Molecular |
| | Repair by | | lipoprotein particle | | Function 2021 |
| | Homologous | | receptor binding | | |
| | Recombination | | | | |
| | Th1 and Th2 | 2.09E−03 | Coronary artery | 4.31E−04 | GWAS Catalog |
| | Activation Pathway | | disease (MI, PTCA, | | 2019 |
| | | | CABG, angina or | | |
| | | | CIHD) | | |
| 13 | Breast Cancer | 3.98E−07 | X-linked severe | 9.14E−10 | Orphanet |
| | Regulation by | | congenital | | Augmented 2021 |
| | Stathmin 1 | | neutropenia | | |
| | CREB Signaling in | 4.57E−07 | X-linked | 9.14E−10 | Orphanet |
| | Neurons | | thrombocytopenia | | Augmented 2021 |
| | | | with normal | | |
| | | | platelets | | |
| | Dopamine- | 5.01E−07 | Vascular | 2.42E−05 | Elsevier Pathway |
| | DARPP32 | | Endothelial Cell | | Collection |
| | Feedback in cAMP | | Vasodilation in | | |
| | Signaling | | Brain | | |
| | Synaptic Long- | 2.24E−06 | regulation of cardiac | 4.68E−05 | GO Biological |
| | Term Potentiation | | muscle contraction | | Process 2021 |
| | P2Y Purigenic | 2.24E−06 | CR3 Mediated | 5.56E−04 | Elsevier Pathway |
| | Receptor Signaling | | Phagocytosis in | | Collection |
| | Pathway | | Neutrophils and | | |
| | | | Macrophages | | |
| 19 | Calcium Signaling | 1.82E−05 | Adipose tissue | 1.41E−06 | Jensen TISSUES |
| | Guanosine | 8.51E−05 | Heart | 2.95E−06 | Jensen TISSUES |
| | Nucleotides | | | | |
| | Degradation III | | | | |
| | Urate | 1.07E−04 | Asparagine N- | 5.84E−06 | BioPlanet 2019 |
| | Biosynthesis/Inosine | | linked glycosylation | | |
| | 5′-phosphate | | | | |
| | Degradation | | | | |
| | Adenosine | 1.62E−04 | Cardiac muscle | 6.33E−06 | Jensen TISSUES |
| | Nucleotides | | | | |
| | Degradation II | | | | |
| | tRNA Splicing | 2.29E−04 | regulation of | 1.78E−05 | GO Biological |
| | | | cytoplasmic mRNA | | Process 2021 |
| | | | processing body | | |
| | | | assembly | | |
| 21 | Regulation Of The | 1.41E−02 | ciliary basal body- | 3.91E−14 | GO Biological |
| | Epithelial | | plasma membrane | | Process 2021 |
| | Mesenchymal | | docking | | |
| | Transition In | | | | |
| | Development | | | | |
| | Granzyme B | 3.24E−02 | cilium assembly | 7.66E−13 | GO Biological |
| | Signaling | | | | Process 2021 |
| | D-myo-inositol | 3.47E−02 | Mitotic G2-G2/M | 8.76E−08 | BioPlanet 2019 |
| | (1,4,5)- | | phases | | |
| | trisphosphate | | | | |
| | Degradation | | | | |
| | 1D-myo-inositol | 3.63E−02 | Genes related to | 2.40E−07 | WikiPathway 2021 |
| | Hexakisphosphate | | primary cilium | | Human |
| | Biosynthesis II | | development | | |
| | (Mammalian) | | | | |
| | D-myo-inositol | 3.63E−02 | Ciliopathies | 3.95E−07 | WikiPathway 2021 |
| | (1,3,4)- | | | | Human |
| | trisphosphate | | | | |
| | Biosynthesis | | | | |
| 33 | nNOS Signaling in | 4.79E−04 | synaptic vesicle | 1.06E−10 | GO Biological |
| | Skeletal Muscle | | exocytosis | | Process 2021 |
| | Cells | | | | |
| | Insulin Secretion | 7.24E−04 | SNARE interactions | 2.95E−06 | BioPlanet 2019 |
| | Signaling Pathway | | in vesicular | | |
| | | | transport | | |
| | Netrin Signaling | 1.07E−03 | regulation of | 3.94E−05 | GO Biological |
| | | | ventricular cardiac | | Process 2021 |
| | | | muscle cell action | | |
| | | | potential | | |
| | Synaptogenesis | 1.12E−03 | Arrhythmogenic | 1.57E−03 | WikiPathway 2021 |
| | Signaling Pathway | | Right Ventricular | | Human |
| | | | Cardiomyopathy | | |
| | Maturity Onset | 1.26E−03 | Cardiac muscle | 1.83E−03 | BioPlanet 2019 |
| | Diabetes of Young | | contraction | | |
| | (MODY) Signaling | | | | |
| 44 | Androgen | 7.94E−15 | steroid biosynthetic | 2.59E−14 | GO Biological |
| | Biosynthesis | | process | | Process 2021 |
| | Estrogen | 7.24E−10 | steroid | 4.27E−14 | GO Molecular |
| | Biosynthesis | | dehydrogenase | | Function 2021 |
| | | | activity, acting on | | |
| | | | the CH—OH group | | |
| | | | of donors, NAD or | | |
| | | | NADP as acceptor | | |
| | Bile Acid | 7.24E−09 | Steroid hormone, | 2.98E−07 | BioPlanet 2019 |
| | Biosynthesis, | | vitamin A, and | | |
| | Neutral Pathway | | vitamin D | | |
| | | | metabolism | | |
| | Estrogen- | 2.00E−06 | Lipid and | 4.51E−07 | BioPlanet 2019 |
| | Dependent Breast | | lipoprotein | | |
| | Cancer Signaling | | metabolism | | |
| | Retinoate | 6.31E−05 | Bile acid and bile | 2.10E−06 | BioPlanet 2019 |
| | Biosynthesis I | | salt biosynthesis via | | |
| | | | 27- | | |
| | | | hydroxycholesterol | | |
| 65 | No enrichment | | Diseases associated | 1.57E−06 | Reactome 2016 |
| | | | with O- | | |
| | | | glycosylation of | | |
| | | | proteins | | |
| | | | metalloendopeptidase | 3.67E−06 | GO Molecular |
| | | | activity | | Function 2021 |
| | | | Thrombospondin | 4.12E−06 | Jensen |
| | | | complex | | COMPARTMENTS |
| | | | extracellular | 6.68E−05 | GO Biological |
| | | | structure | | Process 2021 |
| | | | organization | | |
| | | | TGF-beta signaling | 3.70E−02 | KEGG 2021 |
| | | | pathway | | Human |

TABLE 8B
TIER 2 (MR-IVW causal estimate significant or >7 MR methods
significant): Positive causal estimates pathway analysis by cluster

| | | P. | | P- | |
|---|---|---|---|---|---|
| Cluster | IPA | value | EnrichR | value | Dataset |
| 12 | Death Receptor | 1.48E−07 | Apoptosis | 4.21E−06 | KEGG 2021 Human |
| | Signaling | | | | |
| | Induction of Apoptosis | 5.75E−04 | MAPK Signaling | 9.36E−05 | WikiPathway 2021 |
| | by HIV1 | | Pathway | | Human |
| | Endoplasmic Reticulum | 1.26E−03 | Fas signaling pathway | 3.95E−04 | BioPlanet 2019 |
| | Stress Pathway | | | | |
| | Leukocyte Extravasation | 1.38E−03 | Granzyme A-mediated | 5.73E−04 | BioPlanet 2019 |
| | Signaling | | apoptosis pathway | | |
| | Cardiac Hypertrophy | 2.19E−03 | Lipid and | 3.96E−03 | KEGG 2021 Human |
| | Signaling (Enhanced) | | atherosclerosis | | |
| 14 | FXR/RXR Activation | 1.75E−09 | Mitotic spindle | 2.70E−11 | GO Biological |
| | | | organization | | Process 2021 |
| | | | (GO:0007052) | | |
| | Kinetochore metaphase | 1.36E−08 | Cholesterol transport | 2.00E−06 | GO Biological |
| | signaling pathway | | (GO:0030301) | | Process 2021 |
| | LXR/RXR activation | 3.01E−08 | Cholesterol | 2.30E−08 | KEGG 2021 Human |
| | | | metabolism | | |
| | LPS/IL-1 mediated | 7.47−E06 | High density | 1.37E−10 | GO Cellular |
| | inhibition of RXR | | lipoprotein particle | | Component 2021 |
| | function | | (GO:0034385) | | |
| | Cell cycle control of | 7.10E−05 | Systemic lupus, | 4.66E−07 | GWAS Catalog 2019 |
| | chromosomal replication | | erythematosus | | |
| 15 | No enrichment | | Keratin filament | 1.41E−23 | Jensen |
| | | | | | COMPARTMENTS |
| | | | Intermediate filament | 2.05E−22 | Jensen |
| | | | | | COMPARTMENTS |
| | | | Eukaryotic | 3.11E−02 | BioPlanet 2019 |
| | | | transcription initiation | | |
| | | | channel activity | 3.40E−02 | GO Molecular |
| | | | | | Function 2021 |
| 18 | Assembly of RNA | 2.45E−05 | Transcription by RNA | 1.50E−13 | GO Biological |
| | polymerase II complex | | pol II (GO:0006366) | | Process 2021 |
| | Androgen signaling | 9.01E−04 | Transcription, DNA | 2.60E−10 | GO Biological |
| | | | templated | | Process 2021 |
| | | | (GO:0006351) | | |
| | D-myo-inositol (1,4,5,6)- | 1.79E−02 | Ulcerative colitis | 2.60E−05 | GWAS Catalog 2019 |
| | tetrakisphosphate | | | | |
| | biosynthesis | | | | |
| | D-myo-inositol (3,4,5,6)- | 1.79E−02 | Inflammatory bowl | 3.00E−03 | GWAS Catalog 2019 |
| | tetrakisphosphate | | disease | | |
| | biosynthesis | | | | |
| | 3-phosphoinositide | 2.02E−02 | Lipis traits | 3.00E−03 | GWAS Catalog 2019 |
| | degradation | | | | |
| 23 | iNOS signaling | 1.70E−06 | Regulation of type I | 9.90E−06 | GO Biological |
| | | | interferon production | | Process 2021 |
| | | | (GO:0032479) | | |
| | Role of PKR in | 2.89E−05 | Response to IFN beta | 8.00E−05 | GO Biological |
| | interferon induction and | | (GO:0035456) | | Process 2021 |
| | antiviral response | | | | |
| | Regulation of the EMT | 8.07E−05 | Other acute and | 2.40E−03 | PheWeb2019 |
| | by growth factor | | subacute forms or | | |
| | pathway | | ischemic heart disease | | |
| | TWEAK signaling | 1.28E−04 | Sjogren's syndrome | 5.50E−06 | DisGenNET |
| | HER-2 signaling in | 1.33E−04 | Atherosclerosis | 1.00E−04 | DisGenNET |
| | breast cancer | | | | |
| 28 | Axonal guidance | 1.13E−08 | Semaphorin-plexin | 1.69E−09 | GO Biological |
| | signlaing | | signaling complex | | Process 2021 |
| | | | (GO:0071526) | | |
| | Semaphorin neuronal | 3.76E−08 | Axon guidance | 1.10E−04 | GO Biological |
| | repulsive signaling | | (GO:0007411) | | Process 2021 |
| | pathway | | | | |
| | Circadian rhythm | 2.35E−05 | Hemolytic anemia | 1.60E−03 | Jensen DISEASES |
| | signaling | | | | |
| | PI3K signaling in B | 3.43E−05 | Systemic lupus | 2.00E−03 | Jensen DISEASES |
| | lymphocytes | | erythematosus | | |
| | Epithelial adherens | 4.95E−05 | Cardiovascular system | 6.70E−03 | Jensen DISEASES |
| | junction signaling | | disease | | |
| 31 | Pyrimidine | 5.09E−04 | Ribosomal large | 2.00E−03 | GO Biological |
| | ribonucleotides de novo | | subunit assembly | | Process 2021 |
| | biosynthesis | | (GO″0000027) | | |
| | Coenzyme A | 2.64E−03 | Regulation of | 9.00E−03 | GO Biological |
| | biosynthesis | | complement activation | | Process 2021 |
| | | | (GO0030449) | | |
| | Uridine-5′-phosphate | 2.64E−03 | atypical hemolytic | 5.10E−08 | Clin Var 2019 |
| | biosynthesis | | uremic syndrome | | |
| | Methionine degradation I | 2.09E−02 | Complement and | 5.00E−06 | KEGG 2021 Human |
| | | | coagulation cascades | | |
| | Cystein biosynthesis III | 2.26E−02 | Pyrimidine metabolism | 4.00E−02 | KEGG 2021 Human |
| 34 | Bupropion degradation | 1.04E−02 | Post-golgi vesicle | | GO Biological |
| | | | mediated transport | | Process 2021 |
| | | | (GO:0006892) | | |
| | D-myo-inositol (1,4,5- | 1.08E−02 | Negative regulation of | | |
| | trisphosphate | | insulin signaling | | |
| | biosynthesis | | pathway | | |
| | | | (GO:0046627) | | |
| | Acetone degradation I | 1.08E−02 | Thyroid diseases | 8.00E−03 | GO Biological |
| | | | | | Process 2021 |
| | Estrogen biosynthesis | 1.87E−02 | Diabetes mellitus, | 5.80E−03 | PhenGenI |
| | | | Type I | | Association 2021 |
| | Oxidative ethanol | 2.32E−02 | Obesity related traits | 2.00E−04 | GWAS Catalog 2019 |
| | degradation | | | | |
| 43 | nNOS Signaling in | 1.10E−03 | amyloid-beta metabolic | 1.86E−04 | GO Biological |
| | Skeletal Muscle Cells | | process | | Process 2021 |
| | Synaptogenesis | 3.80E−03 | neutrophil | 2.29E−04 | GO Biological |
| | Signaling Pathway | | degranulation | | Process 2021 |
| | Sperm Motility | 2.75E−02 | neutrophil activation | 2.38E−04 | GO Biological |
| | | | involved in immune | | Process 2021 |
| | | | response | | |
| | Notch Signaling | 3.80E−02 | neutrophil mediated | 2.45E−04 | GO Biological |
| | | | immunity | | Process 2021 |
| | Inhibition of Matrix | 3.89E−02 | EphrinB-EPHB | 7.18E−03 | NCI-Nature 2016 |
| | Metalloproteases | | pathway Homo sapiens | | |
| 48 | CD27 signaling in | 1.43E−02 | Positive regulation of | 1.70E−03 | GO Biological |
| | lympjocytes | | cell motility | | Process 2021 |
| | | | (GO:2000147) | | |
| | RANK signaling in | 2.27E−02 | Positive regulation | 5.00E−03 | GO Biological |
| | osteoclasts | | stem cell proliferation | | Process 2021 |
| | | | (GO:2000648) | | |
| | NGF signaling | 2.98E−02 | Angioedema | 2.00E−03 | PhenGenI |
| | | | | | Association 2021 |
| | IL-15 signaling | 3.05E−02 | Blood glucose | 5.00E−03 | PhenGenI |
| | | | | | Association 2021 |
| | HGF signaling | 3.27E−02 | Plaque, atherosclerotic | 3.00E−02 | PhenGenI |
| | | | | | Association 2021 |
| 51 | No enrichment | | Response to selective | 2.40E−03 | GWAS Catalog 2019 |
| | | | serotonin reuptake | | |
| | | | inhibitors and | | |
| | | | depression | | |
| | | | Metabolite levels | 6.88E−03 | GWAS Catalog 2019 |
| | | | (Pyroglutamine) | | |
| | | | plasma membrane | 8.97E−03 | GO Biological |
| | | | invagination | | Process 2021 |
| | | | phagocytosis, | 1.11E−02 | GO Biological |
| | | | engulfment | | Process 2021 |
| | | | exocytic and synaptic | 1.22E−02 | GO Cellular |
| | | | vesicle membranes | | Component 2021 |
| 60 | GABA Receptor | 9.12E−05 | GABAergic synapse | 8.52E−08 | KEGG 2021 Human |
| | Signaling | | | | |
| | Gustation Pathway | 2.14E−04 | GABA (A) receptor | 9.90E−07 | BioPlanet 2019 |
| | | | activation | | |
| | Neuroinflammation | 5.25E−04 | Ligand-gated ion | 4.50E−06 | BioPlanet 2019 |
| | Signaling Pathway | | channel transport | | |
| | WNT/Ca+ pathway | 8.13E−03 | Cardiac Protection | 1.65E−03 | BioCarta 2016 |
| | | | Against ROS Homo | | |
| | | | sapiens | | |
| | Phospholipases | 8.32E−03 | Vascular tissue | 4.49E−03 | Jensen TISSUES |
| 55 | Sulfite oxidation IV | 0.00117 | GPI anchor metabolic | 7.80E−05 | GO Biological |
| | | | process (GO:0006505) | | Process 2021 |
| | Srine biosynthesis | 0.00352 | Protein lipidation | 8.00E−04 | GO Biological |
| | | | (GO:0006497) | | Process 2021 |
| | Super pathway of serine | 0.00586 | Glycolipid biosyntheic | 1.00E−03 | GO Biological |
| | and glycine biosunthesis | | process (GO:009247) | | Process 2021 |
| | I | | | | |
| | Galactose degradation I | 0.00586 | Glomerular filtration | 5.00E−03 | PhenGenI |
| | | | rate | | Association 2021 |
| | Colanic acid building | 0.0163 | Blood vessel | 2.00E−02 | PhenGenI |
| | blocks biosynthesis | | | | Association 2021 |
| 67 | Glycine cleavage | 2.37E−06 | | | |
| | complex | | | | |
| | Glutamine degradation | 8.38E−04 | | | |
| | Phenylethylamine | 1.68E−03 | | | |
| | degradation I | | | | |
| | Citrulline biosynthesis | 3.77E−03 | | | |
| | y--glutamyl cycle | 5.44E−03 | | | |
| 8 | Th1 and Th2 Activation | 1.26E−11 | Interleukin-27- | 2.17E−16 | BioPlanet 2019 |
| | Pathway | | mediated signaling | | |
| | | | events | | |
| | STAT3 Pathway | 7.08E−10 | NO2-dependent IL-12 | 2.92E−14 | BioPlanet 2019 |
| | | | pathway in NK cells | | |
| | Natural Killer Cell | 5.01E−09 | Behcet Syndrome | 3.54E−10 | DisGeNET |
| | Signaling | | | | |
| | PI3K/AKT Signaling | 5.01E−09 | Selective expression of | 4.47E−10 | WikiPathway 2021 |
| | | | chemokine receptors in | | Human |
| | | | T-cell polarization | | |
| | Cardiac Hypertrophy | 1.07E−08 | Chronic inflammatory | 7.22E−07 | GWAS Catalog 2019 |
| | Signaling (Enhanced) | | diseases (pleiotropy) | | |
| 36 | Phenylethylamine | 3.24E−03 | Type II diabetes | 6.89E−10 | Human Phenotype |
| | Degradation I | | mellitus | | Ontology |
| | RAC Signaling | 5.50E−03 | sensory perception | 1.41E−07 | GO Biological |
| | | | | | Process 2021 |
| | Sphingomyelin | 6.46E−03 | Cell projection | 1.29E−06 | Jensen |
| | Metabolism | | | | COMPARTMENTS |
| | Signaling by Rho Family | 1.95E−02 | USH2 complex | 1.45E−06 | Jensen |
| | GTPases | | | | COMPARTMENTS |
| | Complement System | 2.95E−02 | ligand-gated cation | 2.59E−03 | GO Molecular |
| | | | channel activity | | Function 2021 |

TABLE 8C
TIER 1 (14/16 MR methods significant): Negative causal estimates pathway analysis by cluster.

| | | P- | | P- | |
|---|---|---|---|---|---|
| | IPA | value | EnrichR | value | Dataset |
| 10 | Creatine-phosphate | 6.61E−03 | regulation of keratinocyte | 1.52E−04 | GO Biological |
| | Biosynthesis | | migration | | Process 2021 |
| | Trans, trans-farnesyl | 6.61E−03 | transmembrane receptor | 2.75E−04 | GO Molecular |
| | Diphosphate Biosynthesis | | protein phosphatase | | Function 2021 |
| | | | activity | | |
| | Superpathway of | 2.34E−02 | negative regulation of | 1.34E−03 | GO Biological |
| | Geranylgeranyldiphosphate | | epithelial cell migration | | Process 2021 |
| | Biosynthesis I (via | | | | |
| | Mevalonate) | | | | |
| | Role of Lipids/Lipid Rafts in | 2.95E−02 | Metabolism of Estrogens | 7.73E−03 | Elsevier Pathway |
| | the Pathogenesis of Influenza | | and Androgens | | Collection |
| | Bupropion Degradation | 3.24E−02 | Oxidative Stress | 9.27E−03 | Elsevier Pathway |
| | | | Protection via AHR- | | Collection |
| | | | NFE2L2-NQO1 Signaling | | |
| | | | in Keratinocytes | | |
| 24 | Acyl-CoA Hydrolysis | 4.37E−03 | Fractional exhaled nitric | 4.27E−08 | GWAS Catalog 2019 |
| | | | oxide (childhood) | | |
| | Glutathione Redox Reactions I | 8.51E−03 | Nitric Oxide | 3.11E−07 | PhenGenI |
| | | | | | Association 2021 |
| | IL-17A Signaling in | 1.29E−02 | fatty acid metabolic | 1.33E−03 | GO Biological |
| | Fibroblasts | | process | | Process 2021 |
| | Stearate Biosynthesis I | 1.78E−02 | Artery | 9.37E−03 | Jensen TISSUES |
| | (Animals) | | | | |
| | Apelin Adipocyte Signaling | 2.88E−02 | Cholesterol, HDL | 1.05E−02 | dbGaP |
| | Pathway | | | | |
| 30 | Phospholipases | 1.00E−11 | glycerophospholipid | 1.74E−10 | GO Biological |
| | | | biosynthetic process | | Process 2021 |
| | Choline Biosynthesis III | 2.00E−07 | phosphatidylcholine | 8.26E−09 | GO Biological |
| | | | metabolic process | | Process 2021 |
| | Antioxidant Action of Vitamin | 1.70E−06 | phosphatidylserine | 2.65E−08 | GO Biological |
| | C | | metabolic process | | Process 2021 |
| | Endothelin-1 Signaling | 1.48E−05 | calcium-independent | 4.06E−08 | GO Molecular |
| | | | phospholipase A2 activity | | Function 2021 |
| | Eicosanoid Signaling | 2.04E−05 | phospholipase activity | 6.07E−07 | GO Molecular |
| | | | | | Function 2021 |
| 50 | | | Fc receptor complex | 1.22E−04 | Jensen |
| | | | | | COMPARTMENTS |
| | No enrichment | | Lymphoid cells in Heart | 1.60E−03 | Descartes Cell Types |
| | | | | | & Tissue |
| | | | Coronary atherosclerosis | 2.75E−03 | GWAS Catalog 2019 |
| | | | (increased number of | | |
| | | | diseased vessels) (traffic | | |
| | | | exposure interaction) | | |
| | | | phospholipid scramblase | 3.84E−03 | GO Molecular |
| | | | activity | | Function 2021 |
| | | | Cholesterol, HDL | 3.96E−02 | PhenGenI |
| | | | | | Association 2021 |
| 61 | Dermatan Sulfate Degradation | 1.62E−02 | urate transmembrane | 1.33E−08 | GO Molecular |
| | (Metazoa) | | transporter activity | | Function 2021 |
| | Neuroprotective Role of | 1.09E−01 | urate transport | 1.26E−05 | GO Biological |
| | THOP1 in Alzheimer's Disease | | | | Process 2021 |
| | NRF2-mediated Oxidative | 2.07E−01 | urate metabolic process | 6.91E−05 | GO Biological |
| | Stress Response | | | | Process 2021 |
| | LPS/IL-1 Mediated Inhibition | 2.18E−01 | Abnormality of purine | 3.44E−04 | Human Phenotype |
| | of RXR Function | | metabolism | | Ontology |
| | | | sulfur compound | 9.61E−04 | GO Biological |
| | | | catabolic process | | Process 2021 |

TABLE 8D
TIER 2 (MR-IVW causal estimate significant or >7 MR methods
significant ): causal estimates pathway analysis by cluster.

| | | P- | | P- | |
|---|---|---|---|---|---|
| Cluster | IPA | value | EnrichR | value | Dataset |
| 25 | Synaptogenesis Signaling Pathway | 1.15E−03 | Myocytes | 1.17E−08 | PanglaoDB Augmented |
| | | | | | 2021 |
| | D-myo-inositol (1,4,5,6)- | 2.24E−03 | actomyosin | 2.06E−07 | GO Biological Process |
| | Tetrakisphosphate Biosynthesis | | structure | | 2021 |
| | | | organization | | |
| | D-myo-inositol (3,4,5,6)- | 2.24E−03 | Cardiomyocytes | 5.24E−07 | PanglaoDB Augmented |
| | tetrakisphosphate Biosynthesis | | | | 2021 |
| | Dopamine-DARPP32 Feedback in | 2.45E−03 | Splicing factor | 5.38E−05 | WikiPathway 2021 |
| | CAMP Signaling | | NOVA | | Human |
| | | | regulated | | |
| | | | synaptic | | |
| | | | proteins | | |
| | 3-phosphoinositide Degradation | 2.69E−03 | Biogenic | 3.08E−04 | WikiPathway 2021 |
| | | | Amine | | Human |
| | | | Synthesis | | |
| 45 | Role of | 2.40E−04 | cytokine- | 4.84E−14 | GO Biological Process |
| | Hypercytokinemia/hyperchemokinemia | | mediated | | 2021 |
| | in the Pathogenesis of Influenza | | signaling | | |
| | | | pathway | | |
| | Role of RIG1-like Receptorsin | 2.40E−04 | Jak-STAT | 6.59E−12 | BioPlanet 2019 |
| | Antiviral Innate Immunity | | signaling | | |
| | | | pathway | | |
| | Role of Cytokines in Mediating | 3.31E−04 | Cytokine- | 4.42E−09 | BioPlanet 2019 |
| | Communication between Immune | | cytokine | | |
| | Cells | | receptor | | |
| | | | interaction | | |
| | CNTF Signaling | 3.72E−04 | Complement | 6.15E−03 | MSigDB Hallmark |
| | | | | | 2020 |
| | Activation of IRF by Cytosolic | 4.79E−04 | Reactive | 2.90E−02 | MSigDB Hallmark |
| | Pattern Recognition Receptors | | Oxygen | | 2020 |
| | | | Species | | |
| | | | Pathway | | |
| 66 | PEDF signaling | 2.78E−02 | Histone lysine | 4.50E−05 | GO Biological Process |
| | | | demethylation | | 2021 |
| | | | (GO:0070076) | | |
| | | | Hemolytic | 1.90E−03 | Jensen DISEASES |
| | | | anemia | | |
| | | | Fatty acids, | 1.36E−04 | PhenGenI Association |
| | | | Omega 6 | | 2021 |
| | | | Mean platelet | 1.50E−04 | GWAS Catalog |
| | | | volume | | 2019 |
| | | | Pletelet | 2.70E−06 | DisGeNET |
| | | | aggregation | | |
| | | | Arthritis | 3.90E−06 | GO Molecular |
| | | | (juvenile | | Function 2021 |
| | | | ideopathic) | | |
| 40 | tRNA Splicing | 1.86E−02 | response to | 1.31E−03 | GO Biological Process |
| | | | endoplasmic | | 2021 |
| | | | reticulum | | |
| | | | stress | | |
| | Role of OCT4 in Mammalian | 1.95E−02 | disulfide | 3.99E−03 | GO Molecular |
| | Embryonic Stem Cell Pluripotency | | oxidoreductase | | Function 2021 |
| | | | activity | | |
| | Relaxin Signaling | 6.31E−02 | AMP binding | 5.49E−03 | GO Molecular |
| | | | | | Function 2021 |
| | eNOS Signaling | 6.61E−02 | Nitric oxide | 8.47E−03 | BioPlanet 2019 |
| | | | metabolism | | |
| | Cardiac β-adrenergic Signaling | 7.08E−02 | Vascular | 1.54E−02 | Elsevier Pathway |
| | | | Endothelial | | Collection |
| | | | Cell | | |
| | | | Activation by | | |
| | | | NO | | |

TABLE 8E
Mixed causal estimates

| | | P- | | P- | |
|---|---|---|---|---|---|
| Cluster | IPA | value | EnrichR | value | Dataset |
| 1 | Gai Signaling | 2.00E−14 | G alpha (i) | 7.54E−25 | BioPlanet |
| | | | signaling events | | 2019 |
| | CREB Signaling in Neurons | 5.01E−13 | GPCR ligand | 1.49E−19 | BioPlanet |
| | | | binding | | 2019 |
| | Breast Cancer Regulation by Stathmin1 | 5.01E−11 | Antigen | 4.42E−14 | BioPlanet |
| | | | presentation: | | 2019 |
| | | | folding, | | |
| | | | assembly, and | | |
| | | | peptide loading | | |
| | | | of class I MHC | | |
| | | | proteins | | |
| | cAMP-mediated signaling | 3.31E−09 | Ubiquitin | 2.14E−13 | KEGG 2021 |
| | | | mediated | | Human |
| | | | proteolysis | | |
| | Granulocyte Adhesion and Diapedesis | 1.00E−08 | Chemokine | 1.84E−10 | KEGG 2021 |
| | | | signaling | | Human |
| | | | pathway | | |
| 6 | TH1 and TH2 activation pathway | 1.58E−06 | Epidermis | 1.40E−09 | GO |
| | | | development | | Biological |
| | | | (GO:0008544) | | Process |
| | TH1 pathway | 1.21E−05 | Positive | 1.30E−04 | GO |
| | | | regulation of | | Biological |
| | | | receptor signaling | | Process |
| | | | pathway via | | |
| | | | JAK-STAT | | |
| | | | (GO:0046427) | | |
| | T helper cell differentiation | 1.49E−05 | Cytokine receptor | 2.00E−03 | GO |
| | | | activity | | Molecular |
| | | | (GO:0004896) | | Function |
| | Activation of IRF by cytosolic PRRs | 6.02E−05 | Psoriasis | 4.20E−09 | PhenGenI |
| | | | | | Association |
| | | | | | 2021 |
| | Hepatic fibrosis | 7.45E−05 | Inflammatory | 2.10E−09 | PhenGenI |
| | | | skin diseases | | Association |
| | | | | | 2021 |

Table 9: Lists of SNPs, chromosome locations, p-values and sources (where available).

TABLE 9A
96 SLE and CAD SNPs (FIG. 1). Listed by: SNP ID | Chromosome | Position

rs6589566 | 11 | 116652423; rs662799 | 11 | 116663707; rs12801636 | 11 | 65391317;
rs10774625 | 12 | 111910219; rs653178 | 12 | 112007756; rs17630235 | 12 | 112591686;
rs2304087 | 19 | 11107133; rs6679677 | 1 | 114303808; rs2476601 | 1 | 114377568;
rs2111485 | 2 | 163110536; rs1990760 | 2 | 163124051; rs4248153 | 6 | 31002527;
rs10947129 | 6 | 31011662; rs4713429 | 6 | 31021017; rs3906263 | 6 | 31031904;
rs2394423 | 6 | 31032042; rs12662501 | 6 | 31190850; rs2524054 | 6 | 31252396;
rs2853928 | 6 | 31257511; rs2524142 | 6 | 31263522; rs2247056 | 6 | 31265490;
rs6457374 | 6 | 31272261; rs2523578 | 6 | 31328542; rs2844477 | 6 | 31578772;
rs2857697 | 6 | 31585219; rs2844472 | 6 | 31589676; rs2260050 | 6 | 31591928;
rs2260000 | 6 | 31593476; rs2736171 | 6 | 31595487; rs755714 | 6 | 31609813; rs1077393
| 6 | 31610529; rs805297 | 6 | 31622606; rs521977 | 6 | 31836827; rs605203 | 6 |
31847012; rs589428 | 6 | 31848220; rs659445 | 6 | 31864304; rs644045 | 6 | 31883957;
rs2072633 | 6 | 31919578; rs419788 | 6 | 31928799; rs592229 | 6 | 31930441; rs389883 | 6 |
31947460; rs3134954 | 6 | 32071893; rs2269426 | 6 | 32076499; rs3130283 | 6 |
32138545; rs1800684 | 6 | 32151994; rs3132946 | 6 | 32190028; rs6926737 | 6 |
32375745; rs5007259 | 6 | 32379101; rs6932542 | 6 | 32380262; rs2647012 | 6
32664458; rs2647003 | 6 | 32664880; rs2621416 | 6 | 32741868; rs2857209 | 6 |
32741918; rs2621412 | 6 | 32743000; rs13195441 | 6 | 32743298; rs2857204 | 6 |
32744347; rs13203429 | 6 | 32744852; rs719654 | 6 | 32752139; rs1158784 | 6 |
32758761; rs2199874 | 6 | 32769926; rs2621358 6 | 32770808; rs2621338 | 6 |
32776583; rs2857118 | 6 | 32777900; rs2856997 6 | 32781776; rs2071475 | 6 |
32782387; rs2071474 32782582; rs2071473 6 | 32782605; rs2859579 | 6 |
32784073; rs2071472 | 6 | 32784620; rs2071470 | 6 | 32784753; rs1894408 | 6 |
32786833; rs1894407 | 6 | 32787036; rs2857106 | 6 | 32787570; rs2621323 | 6 |
32788707; rs2621322 | 6 | 32788712; rs2621321 | 6 | 32789480; rs2857103 | 6 |
32791299; rs2856993 | 6 | 32791403; rs2857101 | 6 | 32794676; rs241456 | 6 | 32795965
rs241453 | 6 | 32796226; rs241452 | 6 | 32796346; rs241451 | 6 | 32796480; rs17034 |
6 | 32796521; rs241448 | 6 | 32796685; rs241447 | 6 | 32796751; rs241446 | 6 | 32796967;
rs241445 | 6 | 32797072; rs241443 | 6 | 32797115; rs241441 | 6 | 32797297; rs241440 |
6 | 32797361; rs205262 | 6 | 34563164; rs6457792 | 6 | 34764443; rs9462027 | 6 | 34797241;
rs11755393 | 6 | 34824636; rs6457796 | 6 | 34828553;

TABLE 9B
60 SLE SNPS used as IVs (FIG. 3). Listed by: SNP ID | hg19_{—}
coordinates | hg38_coordinates | trait | study | pmid | ancestry | year | p.

rs17849501 | chr1: 183542323 | chr1: 183573188 | Systemic lupus erythematosus | Bentham
J | 26502338 | European | 2015 | 3.00E−88; rs1234315 | chr1: 173178463 | chr1: 173209324
Systemic lupus erythematosus SLE | Han J W | 19838193 | East Asian | 2009 | 2.34E−26;
rs2732549 | chr11: 35088399 | chr11: 35066852 | Systemic lupus erythematosus | Bentham J
| 26502338 | European | 2015 | 1.00E−23; rs1887428 | chr9: 4984530 | chr9: 4984530 |
Systemic lupus erythematosus | Morris D L | 27399966 | Mixed | 2016 | 2.00E−17;
rs10028805 | chr4: 102737250 | chr4: 101816093 | Systemic lupus erythematosus | Bentham
J | 26502338 | European | 2015 | 4.00E−17; rs9652601 | chr16: 11174365 | chr16: 11080508
Systemic lupus erythematosus | Bentham J | 26502338 | European | 2015 | 7.00E−17;
rs4639966 | chr11: 118573519
chr11: 118702810 | Lupus erythematosus systemic | Han J W
| 19838193 | East Asian | 2009 | 1.00E−16; rs131654 | chr22: 21917190 | chr22: 21562901 |
Lupus erythematosus systemic | Han J W | 19838193 | East Asian | 2009 | 1.00E−16;
rs11185602 | chr7: 50299077 | chr7: 50259481 | Systemic lupus erythematosus | Lessard C J
26663301 | East Asian | 2016 | 2.00E−16; rs6804441 | chr3: 119260944 | chr3: 119542097 |
Systemic lupus erythematosus SLE | Yang W | 23273568 | East Asian | 2012 | 2.50E−16;
rs13385731 | chr2: 33701890 | chr2: 33476823 | Lupus erythematosus systemic | Han J W
19838193 | East Asian | 2009 | 1.00E−15; rs6762714 | chr3: 188470238 | chr3: 188752450
Systemic lupus erythematosus | Morris D L | 27399966 | Mixed | 2016 | 4.00E−15;
rs11073328 | chr15: 38764843 | chr15: 38472642 | Systemic lupus erythematosus
Armstrong D L | 24871463 | European | 2014 | 1.00E−14; rs4852324 | chr2: 74202578
chr2: 73975451 | Systemic lupus erythematosus SLE | Yang W | 23273568 | East Asian |
2012 | 5.70E−14; rs73135369 | chr7: 73940978 | chr7: 74526648 | Systemic lupus
erythematosus | Morris D L | 27399966 | Mixed | 2016 | 9.00E−14; rs1059312
chr12: 129278864 | chr12: 128794319 | Systemic lupus erythematosus | Bentham J |
26502338 | European | 2015 | 1.00E−13; rs11085727 | chr19: 10466123 | chr19: 10355447
Systemic lupus erythematosus | Bentham J | 26502338 | European | 2015 | 1.00E−13;
rs3768792 | chr2: 213871709 | chr2: 213006985 | Systemic lupus erythematosus | Bentham J
| 26502338 | European | 2015 | 1.00E−13; rs10911628 | chr1: 184649503 | chr1: 184680369 |
Systemic lupus erythematosus | Armstrong D L | 24871463 | European | 2014 | 2.00E−13;
rs1564267 | chr8: 11337887 | chr8: 11480378 | Systemic lupus erythematosus SLE | Yang W
| 23273568 | East Asian | 2012 | 3.50E−13; rs7708392 | chr5: 150457485 | chr5: 151077924 |
Systemic lupus erythematosus | Gateva V | 19838195 | European | 2009 | 4.00E−13;
rs6671847 | chr1: 161478810 | chr1: 161509020 | Systemic lupus erythematosus | Bentham J
26502338 | European | 2015 | 1.00E−12; rs34889541 | chr1: 198594769 | chr1: 198625639
Systemic lupus erythematosus | Morris DL | 27399966 | Mixed | 2016 | 2.00E−12; rs597325
chr6: 91002494 | chr6: 90292775 | Systemic lupus erythematosus | Morris D L | 27399966 |
Mixed | 2016 | 4.00E−12; rs548234 | chr6: 106568034 | chr6: 106120159 | Systemic lupus
erythematosus | Han J W | 19838193 | East Asian | 2009 | 5.00E−12; rs7097397
chr10: 50025396 | chr10: 48817351 | Lupus erythematosus systemic | Yang W | 20169177 |
East Asian | 2010 | 8.00E−12; rs4622329 | chr12: 102321935 | chr12: 101928157 | Systemic
lupus erythematosus | Yang W | 23273568 | East Asian | 2012 | 9.00E−12; rs11697848
chr20: 48575315 | chr20: 49958778 | Systemic lupus erythematosus | Armstrong D L |
24871463 | European | 2014 | 1.00E−11; rs3821236 | chr2: 191902758 | chr2: 191038032
Systemic lupus erythematosus | Graham R R | 19165918 | European | 2008 | 8.00E−11;
rs268134 | chr2: 65608363 | chr2: 65381229 | Systemic lupus erythematosus | Bentham J
26502338 | European | 2015 | 1.00E−10; rs58716870 | chr21: 37062330 | chr21: 35690032
Systemic lupus erythematosus | Neale B | UKBB | European | 2017 | 1.68E−10; rs729302
chr7: 128568960 | chr7: 128928906 | Systemic lupus erythematosus SLE females | Harley
J B | 18204446 | European | 2008 | 2.00E−10; rs4902562 | chr14: 68731458 | chr14: 68264741
Systemic lupus erythematosus | Bentham J | 26502338 | European | 2015 | 6.00E−10;
rs4388254 | chr5: 133428601 | chr5: 134092910 | Systemic lupus erythematosus | Bentham J
| 26502338 | European | 2015 | 7.00E−10; rs12802200 | chr11: 566936 | chr11: 566936
Systemic lupus erythematosus | Bentham J | 26502338 | European | 2015 | 9.00E−10;
rs117303757 | chr18: 5628506 | chr18: 5628507 | Systemic lupus erythematosus | Neale B|
UKBB | European | 2017 | 9.72E−10; rs9782955 | chr1: 236039877 | chr1: 235876577
Systemic lupus erythematosus | Bentham J | 26502338 | European | 2015 | 1.00E−09;
rs7579944 | chr2: 30445026 | chr2: 30222160 | Systemic lupus erythematosus | Morris DL |
27399966 | Mixed | 2016 | 1.00E−09; rs115513376 | chr5: 9987678 | chr5: 9987566 |
Systemic lupus erythematosus | Neale B | UKBB | European | 2017 | 1.25E−09; rs564799
chr3: 159728987 | chr3: 160011200 | Systemic lupus erythematosus | Bentham J | 26502338
European | 2015 | 2.00E−09; rs2286672 | chr17: 4712617 | chr17: 4809322 | Systemic lupus
erythematosus | Bentham J | 26502338 | European | 2015 | 3.00E−09; rs74834733 |
chr16: 82504773 | chr16: 82471168 | Systemic lupus erythematosus | Neale B | UKBB
European | 2017 | 4.28E−09; rs12545991 | chr8: 128866115 | chr8: 127853869 | Systemic
lupus erythematosus | Neale B | UKBB | European | 2017 | 5.01E−09; rs7829816
chr8: 56849386 | chr8: 55936827 | Systemic lupus erythematosus SLE females | Harley J B
18204446 | European | 2008 | 5.40E−09; rs494003 | chr11: 65542298 | chr11: 65774827 |
Systemic lupus erythematosus | Morris D L | 27399966 | Mixed | 2016 | 6.00E−09;
rs2941509 | chr17: 37921194 | chr17: 39764941 | Systemic lupus erythematosus | Bentham J
26502338 | European | 2015 | 8.00E−09; rs4917385 | chr10: 105003721 | chr10: 103243964
Systemic lupus erythematosus | Alarcon-Riquelme M E | 26606652 | Hispanic | 2015 |
1.00E−08; rs8023715 | chr15: 97607681 | chr15: 97064451 | Systemic lupus erythematosus
Armstrong D L | 24871463 | European | 2014 | 1.00E−08; rs113342507 | chr7: 123296802
chr7: 123656748 | Systemic lupus erythematosus | Neale B | UKBB | European | 2017 |
1.19E−08; rs145417449 | chr17: 63095664 | chr17: 65099546 | Systemic lupus
erythematosus | Neale B | UKBB | European | 2017 | 1.20E−08; rs34119423 |
chr13: 38687006 | chr13: 38112869 | Systemic lupus erythematosus | Neale B | UKBB
European | 2017 | 1.95E−08; rs12822507 | chr12: 12773521 | chr12: 12620587 | Systemic
lupus erythematosus | Yang W | 23273568 | East Asian | 2012 | 2.00E−08; rs1170426 |
chr16: 68603798 | chr16: 68569895 | Systemic lupus erythematosus | Morris DL | 27399966
| Mixed | 2016 | 2.00E−08; rs1167796 | chr7: 75173180 | - | Systemic lupus erythematosus
Han J W | 19838193 | East Asian | 2009 | 2.00E−08; rs7197475 | chr16: 30642867
chr16: 30631546 | Systemic lupus erythematosus SLE | Han J W | 19838193 | East Asian
2009 | 2.77E−08; rs6590330 | chr11: 128311059 | chr11: 128441164 | Systemic lupus
erythematosus SLE | Yang W | 23273568 | East Asian | 2012 | 3.10E−08; rs117563105 |
chr14: 58524836 | chr14: 58058118 | Systemic lupus erythematosus | Neale B | UKBB
European | 2017 | 3.18E−08; rs146296700 | chr9: 23955500 | chr9: 23955502 | Systemic
lupus erythematosus | Neale B | UKBB | European | 2017 | 3.80E−08; rs144795848
chr11: 130506497 | chr11: 130636602 | Systemic lupus erythematosus | Neale B | UKBB
European | 2017 | 4.37E−08; rs117826817 | chr8: 81015968 | chr8: 80103733 | Systemic
lupus erythematosus | Neale B | UKBB | European | 2017 | 4.89E−08;

TABLE 9C
Positive causal SNPs determined by SSMR (FIG. 6). Listed
by SNP ID | Chromosome | Position.

rs7692514 | 4 | 66142596; rs11037296 | 11 | 43183773; rs75010564 | 5 | 38543824;
rs78506915 | 13 | 44588317; rs73161005 | 22 | 30143240; rs706778 | 10 | 6098949;
rs7810922 | 7 | 101597464; rs965355 | 15 | 60059824; rs12743484 | 1 | 26335929;
rs34099611 | 8 | 144886628; rs11603249 | 11 | 43173812; rs11037278 | 11 | 43173294;
rs11601828 | 11 | 43167522; rs12279248 | 11 | 43178098; rs10905718 | 10 | 6114856;
rs10954650 | 7 | 139326552; rs1609265 | 2 | 229140950; rs13026988 | 2 | 242407179;
rs10933559 | 2 | 242407746; rs73245892 | 4 | 46916798; rs1981601 | 7 | 22202485;
rs2541117 | 12 | 11831208; rs6946131 | 7 | 54783373; rs4869313 | 5 | 96224121;
rs2122775 | 11 | 103399264; rs2594113 | 3 | 71663248; rs7179733 | 15 | 32373693;
rs4459332 | 11 | 72508267; rs209665 | 20 | 32128889; rs874610 | 9 | 100782695;
rs6478522 | 9 | 100790547; rs11164848 | 1 | 93421110; rs11145763 | 9 | 139263596;
rs116967545 | 10 | 64503831; rs10800816 | 1 | 202125363; rs12470957 | 2 | 85773252;
rs6689858 | 1 | 197376021; rs1237290 | 1 | 114135147; rs9851386 | 3 | 174764910;
rs1292043 | 17 | 57934169; rs144484752 | 14 | 37439749; rs16837131 | 2 | 202173812;
rs13023380 | 2 | 163154363; rs2019097 | 2 | 202193463; rs62131887 | 19 | 10587596;
rs6738825 | 2 | 198896895; rs425648 | 19 | 18202112; rs2022013 | 1 | 183353853;
rs2456973 | 12 | 56416928; rs223881 | 16 | 57386566; rs56114296 | 17 | 26110748;
rs1534154 | 3 | 119311030; rs10916668 | 1 | 20233086; rs3807134 | 7 | 128577338;
rs131076124 | 102739980; rs13106926 | 4 | 102739791; rs34749007 | 4 | 102754507;
rs353880914 | 102739452; rs4637409 | 4 | 102753408; rs34029191 | 4 | 102752201;
rs172665944 | 102750922; rs1125271 | 4 | 102745985; rs17200824 | 4 | 102752589;
rs13129744 | 4 | 102756731; rs10516487 | 4 | 102751076; rs12119966 | 1 | 183378051;
rs34725611 | 19 | 10477067; rs11085727 | 19 | 10466123; rs2078087 | 1 | 183358405;
rs12409487 | 1 | 183345350; rs1122259 | 1 | 183356931; rs4411998 | 4 | 102738147;
rs11150610 | 16 | 31334236; rs34815476 | 7 | 128563333; rs11150608 | 16 | 31301125;
rs729302 | 7 | 128568960; rs2061831 | 8 | 11339882; rs13426947 | 2 | 191933254;
rs10168266 | 2 | 191935804; rs10931481 | 2 | 191954852;

TABLE 9D
Negative causal SNPs determined by SSMR (FIG. 6). Listed
by SNP ID | Chromosome | Position.

rs6756736 | 2 | 43558743; rs12946196 | 17 | 75807910; rs8105429 | 19 | 18481333;
rs34709472 | 19 | 55704944; rs4678000 | 3 | 121890735; rs7566072 | 2 | 236203142;
rs10878246 | 12 | 40632099; rs4917385 | 10 | 105003721; rs4748857 | 10 | 23599558;
rs928596 | 1 | 155947875; rs6449173 | 4 | 9966105; rs7442295 | 4 | 9966380; rs73066671 |
19 | 55696684; rs7821169 | 8 | 59187800; rs1052690 | 9 | 86258685; rs3785437 | 17 |
73906222; rs7540556 | 1 | 169531442; rs11057864 | 12 | 125335950; rs7915387 | 10
73010024; rs1053093 | 1 | 183905073; rs144705368 | 2 | 10642477; rs7927370 | 11 |
55136219; rs3766374 | 1 | 160720554; rs7121755 | 11 | 115455636; rs7899961 | 10 |
60026425; rs6945400 | 7 | 51079850; rs3733345 | 4 | 954247; rs4293757 | 4 | 30943209;
rs55816332 | 7 | 31423490; rs500600 | 11 | 30481057; rs79821347 | 3 | 50531835;
rs12683801 | 9 | 5272381; rs7214635 | 17 | 4601020; rs9415635 | 10 | 63753482;
rs55634455 | 2 | 136593694; rs10419198 | 19 | 50038017; rs138191786 | 7 | 27415597;
rs9568353 | 13 | 50481713; rs9952980 | 18 | 42888797; rs79558495 | 7 | 100365435;
rs1372372 | 1 | 30120947; rs16953685 | 15 | 70034088; rs72832915 | 17 | 37834808;
rs2235947 | 20 | 8628696; rs35281701 | 15 | 69992054; rs4948496 | 10 | 63805617;
rs7625006 | 3 | 51372940; rs7264711 | 20 | 8630944; rs55705316 | 1 | 206933517;
rs6574349 | 14 | 77497666; rs118087357 | 20 | 54465778; rs2941509 | 17 | 37921194;
rs6705304 | 2 | 43596746; rs28392589 | 17 | 37992885; rs12946188 | 17 | 37984430;
rs113115305 | 17 | 37970687; rs113370572 | 17 | 37933468; rs12946792 | 17 | 37985322;
rs12978179 | 19 | 2183175; rs112345383 | 17 | 37942018; rs600751 | 11 | 126059460;
rs28621669 | 17 | 38011266; rs28723017 | 17 | 38011451; rs12947480 | 17 | 38009676;
rs12938117 | 17 | 38008165; rs8079075 | 17 | 38010815; rs111469562 | 17 | 37982697;
rs113233720 | 17 | 38008191; rs112880843 | 17 | 38024298; rs4252665 | 17 | 37885383;
rs143123127 | 17 | 38009000; rs9907966 | 17 | 38018803; rs1453560 | 17 | 38023441;
rs1131665 | 11 | 613208; rs35865896 | 11 | 584591; rs12272314 | 11 | 590648; rs1055382 |
11 | 612182; rs1061502 | 11 | 614318; rs34889521 | 11 | 617519; rs11246213 | 11 | 612967;
rs12805435 | 11 | 612355; rs11246217 | 11 | 623765; rs58688157 | 11 | 625085; rs1124816
11 | 619671; rs11539530 | 11 | 609353; rs36060383 | 11 | 598723; rs2396545 | 11 | 601785;
rs936469 | 11 | 606749; rs4963128 | 11 | 589564; rs12795572 | 11 | 590999; rs12419694
11 | 598033; rs10229001 | 7 | 128599397; rs10954213 | 7 | 128589427; rs10239340 | 7 |
128668510; rs4731536 | 7 | 128640293; rs4731533 | 7 | 128602142;

TABLE 9E
838 Immunochip SNPs (FIGS. 2, 9 and 11)

rs12574073; rs56886418; rs66941367; rs10905718; rs1453560; rs1499077; rs1565901;
rs180977001; rs2298455; rs3731570; rs452999; rs4648892; rs4946810; rs5029939;
rs513425; rs57095329; rs62097857; rs653215; rs6715106; rs6889239; rs936126;
rs9965182; rs1559758; rs2431697; rs4731532; rs7002136; rs10798176; rs11117431;
rs11788118; rs12588573; rs1707603; rs2431098; rs2431697; rs268794; rs4242897;
rs4917014; rs55869446; rs56272032; rs6574349; rs6651252; rs688161; rs7011788;
rs74597067; rs7726414; rs913678; rs965355; rs10057690; rs10181656; rs1033177;
rs10405730; rs10496255; rs10498444; rs10762171; rs11087739; rs11763959; rs12039904;
rs12065145; rs12065623; rs12261850; rs12537284; rs13014054; rs1433905; rs1450712;
rs146250785; rs149495769; rs1550250; rs16826235; rs17021444; rs17133826;
rs17266594; rs17552904; rs17570297; rs17690923; rs1804182; rs1946007; rs2071929;
rs2117209; rs2553878; rs2594113; rs2618458; rs2736340; rs2940707; rs34840245;
rs3757387; rs3792615; rs41308952; rs56194674; rs58395088; rs591796; rs6681482;
rs6691730; rs7032238; rs7582300; rs77029323; rs8132092; rs850548; rs886663;
rs9408808; rs9851386; rs10085092; rs10118244; rs1012172; rs1012525; rs10197010;
rs10197379; rs10202434; rs10209543; rs1024580; rs10411749; rs10483515; rs10486135;
rs10492224; rs10496726; rs10506577; rs10510388; rs1051624; rs1052690; rs1053093;
rs10753074; rs10760059; rs10761901; rs10773782; rs10818375; rs10819689; rs10850369;
rs10868841; rs10889449; rs10931167; rs11050953; rs11065513; rs11074015; rs11076230;
rs11096566; rs11134150; rs11134275; rs11136577; rs11164848; rs11166634; rs11171;
rs11177019; rs11181779; rs112123005; rs1121242; rs11231814; rs11235715; rs11246217;
rs1131265; rs113304138; rs1133950; rs113420846; rs113815962; rs113867041;
rs114568253; rs114800103; rs115291397; rs115333320; rs11539148; rs11578098;
rs116101946; rs11612569; rs11616341; rs11635120; rs11640251; rs11673460;
rs116818286; rs11681903; rs116844988; rs116912920; rs116967545; rs11705804;
rs11722709; rs11724582; rs117297579; rs11750159; rs117626207; rs117836922;
rs11788947; rs11791699; rs11792269; rs11803383; rs11887156; rs1189433; rs1193329;
rs11998187; rs12142199; rs12145347; rs12148050; rs12156002; rs12207692; rs12211203;
rs12281485; rs12290504; rs12307984; rs12322502; rs12408855; rs12417865; rs12444634;
rs12464087; rs12474299; rs12524486; rs12526490; rs12551731; rs12575600; rs12597935;
rs12613330; rs12666575; rs1267489; rs12683801; rs12692144; rs12706861; rs12712067;
rs12722558; rs12741962; rs12742128; rs12764378; rs12865863; rs12916690; rs12918180;
rs12946196; rs12971295; rs12978179; rs13001747; rs13023380; rs13026988; rs13095186;
rs13126505; rs13172944; rs13277113; rs13383488; rs13384308; rs13394788; rs1340237;
rs13410482; rs13425999; rs1350176; rs1351161; rs1355208; rs1360328; rs1361925;
rs1369481; rs1372372; rs138584427; rs1393988; rs1435649; rs1470475; rs1472788;
rs1473176; rs147622113; rs150173969; rs1546913; rs1565123; rs1609265; rs164178;
rs1647960; rs1670807; rs16858884; rs16864190; rs16867384; rs169080; rs16935357;
rs16946868; rs16953685; rs16991802; rs17075185; rs17100664; rs17105765; rs17160068;
rs17335437; rs17362529; rs17413405; rs17483887; rs17484292; rs1751036; rs17546779;
rs17610618; rs17681910; rs17759986; rs17779870; rs17805636; rs17813747; rs1801274;
rs1860598; rs1864975; rs1869486; rs1894147; rs1913517; rs1922240; rs1952537;
rs1958442; rs1974876; rs1975197; rs1981601; rs1983208; rs1990313; rs1997392;
rs2015407; rs2019097; rs2026995; rs203102; rs2044264; rs2055802; rs2057016;
rs2061776; rs2082418; rs2083146; rs2095731; rs209677; rs2109842; rs2111485;
rs2122775; rs2136349; rs2156832; rs2161070; rs2205960; rs2220168; rs2227203;
rs2228054; rs2230926; rs2235947; rs2238573; rs223883; rs2283293; rs2287339;
rs2299864; rs2303108; rs2325399; rs2327832; rs238353; rs2425223; rs2456973;
rs2460382; rs2489159; rs2499714; rs2541117; rs255915; rs255920; rs2576837;
rs2600694; rs2624217; rs2654676; rs2663054; rs2726101; rs2787733; rs2804278;
rs285049; rs285050; rs2891551; rs2891663; rs2958647; rs2959219; rs3109154;
rs3122605; rs33421; rs34006827; rs34536443; rs34572943; rs34725611; rs34743561;
rs35281701; rs35370245; rs35667974; rs357999; rs364418; rs3739817; rs376072;
rs3766374; rs3773377; rs3780205; rs3801814; rs3818246; rs3829578; rs3862550;
rs415407; rs422258; rs424561; rs4283545; rs4293757; rs4298479; rs4355385; rs436872;
rs4369667; rs4375990; rs4426778; rs4459332; rs4469464; rs4476350; rs4513082;
rs4515165; rs45444697; rs4547082; rs4552888; rs4573125; rs4579751; rs4610334;
rs461193; rs463149; rs4666298; rs4678000; rs4686912; rs4708114; rs4719714;
rs4777427; rs4783754; rs4796445; rs4811174; rs4844600; rs4859711; rs4869426;
rs4885144; rs4921317; rs4927193; rs4930194; rs4936059; rs4942903; rs4944946;
rs4971066; rs497356; rs4974949; rs501192; rs507230; rs511580; rs526151; rs548234;
rs55634455; rs55873962; rs559311; rs56042874; rs560596; rs56114296; rs564976;
rs57545959; rs58089667; rs59494035; rs6032198; rs6073958; rs6126200; rs6133922;
rs61794854; rs61883873; rs62057174; rs62136099; rs631574; rs633040; rs6449173;
rs6478522; rs6499034; rs17617; rs6565201; rs6579789; rs6581430; rs6581431;
rs6586128; rs6595788; rs6673121; rs6673129; rs6680666; rs6700189; rs6705304;
rs6743585; rs6756736; rs6756819; rs6780105; rs6857709; rs6869688; rs6886392;
rs6912394; rs6913643; rs6945305; rs6945400; rs6950918; rs696780; rs7104239;
rs7114131; rs7114911; rs7121755; rs7150563; rs71606243; rs7179733; rs7240797;
rs724710; rs7248181; rs7252175; rs7264711; rs72768698; rs72807059; rs72832915;
rs72928038; rs72941674; rs73029013; rs7311756; rs73245892; rs7325747; rs73286503;
rs73440898; rs7359953; rs73683966; rs73966422; rs7439087; rs7442295; rs7486855;
rs75096128; rs7566072; rs7566538; rs7568275; rs75926644; rs7601533; rs760583;
rs7606861; rs7633180; rs7639882; rs7692950; rs77000060; rs7714375; rs7720046;
rs7759743; rs7764429; rs7770235; rs7810922; rs78209349; rs7821169; rs7823506;
rs7826510; rs7829816; rs78318981; rs78481160; rs78712140; rs7872388; rs7900818;
rs7916519; rs7939069; rs79399781; rs7946044; rs79558495; rs79821347; rs7982251;
rs7982957; rs80179578; rs8048448; rs8061145; rs8079075; rs8085075; rs8116375;
rs816685; rs835574; rs849142; rs867709; rs874610; rs882829; rs888656; rs894956;
rs905443; rs907715; rs909788; rs910425; rs916287; rs926571; rs9283855; rs928596;
rs9303891; rs932169; rs9353321; rs9366394; rs9373839; rs9415635; rs9427315;
rs9428886; rs944333; rs9476757; rs948018; rs9520836; rs9568353; rs962885; rs9651118;
rs9783215; rs9784212; rs9821576; rs9823523; rs9827931; rs9855205; rs9861188;
rs9880289; rs9884026; rs9887785; rs9887904; rs989767; rs9952980; rs10516487;
rs1143679; rs6923608; rs7582694; rs7708392; rs4802307; rs6131014; rs600751;
rs116660017; rs12743484; rs41263646; rs425648; rs6088765; rs6492983; rs12114284;
rs12664535; rs1378942; rs1966115; rs2244364; rs2732549; rs2816317; rs352834;
rs4731532; rs726848; rs7812879; rs10402677; rs10823062; rs10878246; rs1127745;
rs11571293; rs13061150; rs13399274; rs224031; rs2389683; rs2407782; rs26657;
rs4657019; rs4845473; rs4958136; rs602097; rs632111; rs72793687; rs73039184;
rs7899961; rs8061370; rs8128037; rs1016350; rs10197217; rs10271373; rs10498722;
rs1072626; rs10800816; rs10916668; rs11647067; rs11658698; rs11681718; rs11738358;
rs11756587; rs12050151; rs12150495; rs12248123; rs12298456; rs1237290; rs12738833;
rs13000817; rs13272330; rs1410454; rs1539414; rs16837131; rs17036143; rs17079296;
rs183211; rs2110258; rs2229524; rs223881; rs2241003; rs2244272; rs2277802; rs230261;
rs2304087; rs238224; rs243323; rs2600669; rs2723196; rs2732159; rs2823288;
rs2955587; rs2982846; rs351758; rs4580644; rs4644492; rs4712969; rs4810485;
rs4888360; rs4948334; rs533852; rs562954; rs62149377; rs6589566; rs6738825;
rs7214635; rs7249065; rs7280984; rs72952888; rs73137125; rs73366469; rs7580177;
rs7764323; rs7808907; rs7831557; rs8066255; rs904661; rs907526; rs9311929;
rs9462027; rs9652601; rs10225904; rs1039917; rs10419198; rs1059312; rs1061511;
rs10735418; rs10800309; rs10889681; rs10889687; rs10922144; rs10933559; rs10952261;
rs11024600; rs11059927; rs11070264; rs11089629; rs11130633; rs11145974; rs11154801;
rs1132200; rs11598094; rs11671087; rs11685321; rs1170436; rs11705183; rs11755393;
rs11763959; rs11804305; rs1194; rs12096737; rs12129787; rs12422045; rs12531540;
rs12599402; rs12801636; rs1292043; rs12986413; rs12992815; rs13123614; rs13205210;
rs13252899; rs13344313; rs1385374; rs1413753; rs1441904; rs1463485; rs1496453;
rs1534154; rs16979595; rs17630235; rs196432; rs1983891; rs209665; rs2230776;
rs2256597; rs2271293; rs2380925; rs2396545; rs2455331; rs2456449; rs2476601;
rs266433; rs2675954; rs2688608; rs281392; rs2928403; rs2953898; rs34099611;
rs34840245; rs35000415; rs3733345; rs3743408; rs3769433; rs3769503; rs3785437;
rs3790564; rs3807307; rs3864261; rs3925535; rs4234726; rs4252665; rs4363498;
rs4474733; rs4482697; rs454425; rs464694; rs4690055; rs4707585; rs4963128; rs500600;
rs501089; rs56154925; rs56228409; rs62262116; rs6445975; rs645078; rs6477694;
rs653178; rs6557142; rs6598011; rs6601327; rs662799; rs6662618; rs6663448;
rs6679677; rs6920432; rs6923307; rs6938946; rs6980682; rs702814; rs7186889;
rs7241191; rs72799341; rs73066671; rs73161005; rs746179; rs74908652; rs7500321;
rs750402; rs7522008; rs7533588; rs7540556; rs7625006; rs763616; rs7679; rs7712962;
rs771459; rs7787; rs78506915; rs79074797; rs7915387; rs7970893; rs930297; rs9367646;
rs968567; rs9894206;

TABLE 9F
914 Phenoscanner SNPs (FIGS. 2, 9 and 11)

rs10028805; rs10036748; rs10041935; rs10048743; rs1012507; rs10142203; rs10152577;
rs10156091; rs10168266; rs10239340; rs10254284; rs10276619; rs10279821; rs10282742;
rs1028488; rs1031154; rs10466455; rs1048257; rs10483546; rs10488631; rs10489265;
rs1049564; rs10498070; rs10499197; rs10507521; rs10516487; rs10521318; rs1059312;
rs1061502; rs10737562; rs10742326; rs10753074; rs10774625; rs10798269; rs10822050;
rs10845606; rs10847697; rs10857650; rs10857712; rs10893872; rs10903340; rs10911363;
rs10911390; rs10911628; rs10912578; rs10931481; rs10954650; rs10988542; rs10991892;
rs10992211; rs10995092; rs10998903; rs10998904; rs10998905; rs10998908; rs11013210;
rs11037278; rs11037296; rs11057864; rs11059919; rs11073328; rs1108131; rs11085727;
rs11101442; rs111230937; rs111280245; rs111319527; rs11145763; rs111476894;
rs11150610; rs111546231; rs111737889; rs111744793; rs111758635; rs111835936;
rs11185602; rs11185603; rs112018349; rs112058316; rs112224044; rs11231824;
rs112329115; rs112430375; rs112435140; rs112455696; rs112529123; rs11259145;
rs11259205; rs112610233; rs11264697; rs112760845; rs1128334; rs113061650;
rs113095313; rs113205188; rs113245606; rs113386660; rs1133906; rs113392673;
rs113419431; rs113506094; rs113707668; rs113743276; rs113761352; rs113775192;
rs113779281; rs113780289; rs113849850; rs113870709; rs114056799; rs114209701;
rs114273055; rs1143678; rs1143679; rs1143683; rs114628184; rs114742305;
rs114791303; rs114802133; rs114846446; rs115073033; rs115081579; rs115513376;
rs11574637; rs115765118; rs11580078; rs11601828; rs11603249; rs116104528;
rs116144510; rs116205614; rs116302911; rs116416688; rs11644034; rs11655550;
rs11662413; rs116658618; rs11668540; rs116703756; rs116804160; rs116964910;
rs11697848; rs116987560; rs117003280; rs117036242; rs1170426; rs117174224;
rs11717455; rs117196351; rs117206569; rs117222934; rs117261473; rs117301471;
rs117303757; rs117325149; rs117371650; rs117372389; rs117382357; rs11741255;
rs117492752; rs117519683; rs117528104; rs117563105; rs117593252; rs117610755;
rs117658872; rs117682184; rs117692083; rs117818071; rs117826817; rs117830658;
rs11783247; rs11790378; rs11793585; rs118087357; rs118128424; rs11813373;
rs118152395; rs118164679; rs11839053; rs11842000; rs11860650; rs11866427;
rs11889341; rs11951576; rs12039812; rs12108299; rs12141391; rs12189434; rs12232497;
rs12235805; rs12268734; rs12279248; rs12284542; rs12312476; rs1234315; rs12415402;
rs12444486; rs12470957; rs12500106; rs12500188; rs12501889; rs12503080; rs12504328;
rs1250563; rs12506429; rs12506703; rs12509044; rs12509545; rs12512006; rs12529935;
rs12531540; rs12531711; rs12537284; rs12539741; rs12541115; rs12545991; rs12546286;
rs12565776; rs12589674; rs12598357; rs12599402; rs12612769; rs12620999; rs12626541;
rs12627125; rs12627213; rs12629106; rs12657174; rs12677326; rs12680762; rs12711490;
rs12736195; rs12742494; rs12753920; rs12802200; rs12822507; rs12894741; rs12917712;
rs12928404; rs12949531; rs12992463; rs12993006; rs131654; rs13168551; rs1321681;
rs13239597; rs13257071; rs13277113; rs1332099; rs13332649; rs13333835; rs13385731;
rs13418915; rs13439530; rs1355223; rs1364989; rs137870785; rs137956473;
rs138027232; rs138191786; rs138384991; rs138408824; rs138417115; rs1385374;
rs138759056; rs138770919; rs138801602; rs138912977; rs139015054; rs139052917;
rs139278659; rs139376595; rs139383914; rs139394049; rs139539455; rs139737241;
rs139810877; rs139831076; rs139843719; rs140207360; rs140281463; rs140351402;
rs140380896; rs140541658; rs140557893; rs140795730; rs140870671; rs140959054;
rs140976895; rs141136751; rs141162791; rs141529969; rs141691106; rs141868266;
rs141941919; rs141947161; rs141957463; rs142099231; rs142105249; rs142176736;
rs142512991; rs142608977; rs142666415; rs1429411; rs143040080; rs143123127;
rs143269888; rs143583573; rs143702273; rs143810807; rs144231913; rs144270413;
rs144401070; rs144484752; rs144526725; rs144705368; rs144795848; rs144959063;
rs145195496; rs145409643; rs145417449; rs145595328; rs145691683; rs145698618;
rs145768357; rs145858995; rs145945915; rs145977671; rs145992193; rs146041145;
rs146104365; rs146296700; rs1463525; rs146366492; rs1464776; rs146506150;
rs146649068; rs146714357; rs147173503; rs147337990; rs147355425; rs147484050;
rs147558730; rs1478897; rs148132289; rs148235286; rs148274675; rs149002524;
rs149085746; rs149214948; rs149586506; rs149756664; rs149940602; rs150128358;
rs150256879; rs150389346; rs150568573; rs150677691; rs150719555; rs150739866;
rs1509461; rs151156293; rs151290905; rs1534618; rs1564267; rs1570898; rs1576748;
rs1635852; rs16833239; rs16840423; rs16842284; rs16860537; rs1689510; rs16919874;
rs16919876; rs16926583; rs16993569; rs16993576; rs16993577; rs16993578; rs16993582;
rs16993583; rs16993585; rs17026372; rs17026452; rs17039212; rs17065161; rs17083844;
rs17103280; rs17103282; rs17113620; rs17114134; rs17124948; rs17130394; rs17266594;
rs17301013; rs17319190; rs17319672; rs17321999; rs17462017; rs17466626; rs17497148;
rs17571812; rs17602569; rs17602639; rs17653353; rs17807624; rs17849501; rs17885785;
rs1801274; rs181543137; rs181550871; rs182155942; rs182475325; rs182643381;
rs182656; rs182961757; rs1830847; rs1830848; rs183964972; rs184775961; rs1847896;
rs1853689; rs185554382; rs1861525; rs186479757; rs1874791; rs187577845; rs1876680;
rs188721350; rs1887428; rs189080859; rs189257248; rs190479475; rs190582650;
rs190967129; rs1913517; rs192127391; rs192310906; rs1990760; rs2022013; rs2039086;
rs2066363; rs2070197; rs2071725; rs2075184; rs2111485; rs2121037; rs2176082;
rs2205960; rs2222631; rs2230926; rs2236178; rs2242781; rs2242782; rs2242785;
rs2248932; rs2252996; rs2254546; rs2267828; rs2275247; rs2283790; rs2286672;
rs2288012; rs2289583; rs2297550; rs2303745; rs2304256; rs2313132; rs2325088;
rs2384534; rs2396545; rs2431098; rs2431099; rs2431697; rs2476601; rs2478118;
rs2501800; rs2609881; rs2618476; rs2618479; rs2663052; rs2667978; rs2669010;
rs268134; rs2732549; rs2736332; rs2736340; rs2736345; rs274068; rs2785197;
rs2786111; rs2834998; rs2835106; rs2835120; rs2836882; rs2914727; rs2928402;
rs2941509; rs2963864; rs2972956; rs2973927; rs2973929; rs3024493; rs3024505;
rs3097841; rs3111095; rs330048; rs340630; rs34119423; rs34330; rs34432903;
rs34441860; rs34572943; rs34709472; rs34884278; rs34889541; rs35000415; rs35294273;
rs35355603; rs35472514; rs35853026; rs36001488; rs36051895; rs3747093; rs3757173;
rs3757387; rs3768792; rs3783759; rs3794060; rs3821236; rs3827644; rs3862260;
rs387619; rs3914167; rs3934007; rs41452648; rs41478451; rs4149228; rs4240671;
rs4246905; rs424962; rs427221; rs428073; rs4388254; rs4468247; rs4522865; rs4544377;
rs4548893; rs4574684; rs4622329; rs4625; rs463426; rs4639966; rs4641121; rs4660116;
rs4676410; rs4684256; rs4728142; rs4748857; rs4748940; rs4766921; rs4773785;
rs4821112; rs4822648; rs4822649; rs4850410; rs4852324; rs4869313; rs4901847;
rs4902562; rs4910907; rs4916342; rs4917014; rs4917385; rs4921283; rs4925043;
rs494003; rs4942249; rs4948496; rs4956211; rs4958893; rs4958894; rs4963128;
rs497273; rs5029924; rs5029937; rs5029939; rs503425; rs525410; rs548234; rs55705316;
rs55816332; rs56197414; rs56262793; rs564799; rs573775; rs5754217; rs5760894;
rs5760898; rs57681844; rs57956751; rs58688157; rs58716870; rs58721818; rs59031468;
rs59034468; rs59441655; rs59507468; rs59657785; rs597325; rs597808; rs59953583;
rs601162; rs602662; rs60288561; rs6049839; rs6067571; rs6084875; rs61127290;
rs61209988; rs61814766; rs61919512; rs61959960; rs61959998; rs619796; rs62104772;
rs62130302; rs62131887; rs62276340; rs62282423; rs62282430; rs62324212; rs62361935;
rs6438700; rs6445975; rs6486730; rs6538678; rs6568431; rs6590330; rs6592199;
rs6601327; rs6671847; rs6679677; rs6689858; rs6695567; rs6696619; rs6697139;
rs670369; rs6705628; rs6736175; rs6742110; rs6762714; rs6804441; rs6814866;
rs6850606; rs6889239; rs6914831; rs6916001; rs6920220; rs6932056; rs6946131;
rs6964720; rs6984496; rs6985109; rs7013627; rs7014428; rs7031325; rs7042370;
rs704840; rs704853; rs706778; rs7089307; rs7090925; rs7092317; rs7094610; rs7094957;
rs7097397; rs7100025; rs710987; rs7116618; rs71304622; rs71306055; rs71427120;
rs71606110; rs7172677; rs7186852; rs7197475; rs7200786; rs7258015; rs72739935;
rs72743477; rs7277126; rs72787037; rs72820662; rs72820663; rs72841703; rs72841707;
rs729302; rs72938590; rs73135369; rs73152525; rs73152987; rs73156242; rs73170472;
rs73209462; rs73209465; rs73209470; rs73211351; rs73211357; rs73211366; rs73211381;
rs73211383; rs73211388; rs73211394; rs73211401; rs73213404; rs73213405; rs73213406;
rs73213408; rs73213413; rs73223242; rs73223246; rs73223250; rs73223282; rs73223294;
rs7333671; rs73365739; rs73365744; rs73365745; rs73365749; rs73365750; rs73365755;
rs73365764; rs73365765; rs73365766; rs73365769; rs73365771; rs73365774; rs73365775;
rs73365797; rs73458378; rs73599338; rs73693512; rs73693535; rs73693536; rs73881943;
rs74068700; rs7411387; rs74157232; rs742108; rs74392266; rs74392337; rs74420063;
rs7444; rs74546614; rs74603582; rs74623793; rs74649380; rs74717727; rs74742895;
rs74760083; rs74782705; rs74789601; rs74818302; rs74834733; rs74876140; rs749231;
rs7496663; rs74974648; rs75010564; rs75092674; rs75102897; rs751437; rs751438;
rs752010; rs75234951; rs75250964; rs755374; rs75695234; rs7574865; rs7575676;
rs75784138; rs7579944; rs75826569; rs7582694; rs7584501; rs7590355; rs7594501;
rs75946926; rs7601754; rs76090800; rs76114932; rs76238820; rs76392962; rs76472582;
rs76521500; rs76594721; rs7660520; rs76608785; rs76678040; rs7672495; rs76814707;
rs7683537; rs76848381; rs76852463; rs76859420; rs76918338; rs7692514; rs77037666;
rs7708392; rs77146244; rs77150043; rs7725052; rs7726414; rs7731626; rs77481934;
rs7749323; rs77528423; rs77743832; rs77765852; rs77825805; rs77850688; rs77966013;
rs77999417; rs7808907; rs7812879; rs7820492; rs7824834; rs7825948; rs7829816;
rs7831697; rs7834765; rs7836059; rs78373675; rs78374842; rs78422526; rs78484424;
rs78573504; rs78609563; rs78656115; rs78720321; rs78921499; rs7897633; rs79007842;
rs79008672; rs79087942; rs79154635; rs7922169; rs79234185; rs79260145; rs7927370;
rs79294954; rs79404002; rs79405392; rs7941765; rs7949209; rs79836876; rs79877921;
rs7989702; rs7995955; rs8009420; rs8011382; rs8012283; rs80177654; rs80186485;
rs80193959; rs80209682; rs8023715; rs80247019; rs80313423; rs8105429; rs844648;
rs849142; rs870707; rs877819; rs912784; rs918959; rs921916; rs9298502; rs9303277;
rs931127; rs9311676; rs9398235; rs9402743; rs9482510; rs9524400; rs9554399;
rs9554402; rs9556721; rs9556722; rs9556732; rs9556734; rs956237; rs9582130;
rs958476; rs959260; rs9603612; rs960709; rs9634496; rs9652601; rs9658386; rs9658397;
rs9658480; rs9658527; rs9658550; rs9658574; rs967616; rs9751373; rs9782955;
rs979233; rs9888739; rs9937837;

Table 13: SNPs within, mapped genes and the associated functional annotation of, the 67 SNP clusters.

TABLE 13-1
Cluster 1 of the 67 SNP clusters.

| | |
|---|---|
| Functional | Gαi Signaling |
| Annotation | CREB Signaling in Neurons |
| | Breast Cancer Regulation by Stathmin1 |
| | cAMP-mediated signaling |
| | Granulocyte Adhesion and Diapedesis |
| | G alpha (i) signaling events |
| | GPCR ligand binding |
| | Antigen presentation: folding, assembly, and peptide loading of class I MHC |
| | proteins |
| | Ubiquitin mediated proteolysis |
| | Chemokine signaling pathway |
| SNPs | rs11697848, rs2286672, rs238224, rs2071725, rs7249065, rs9821576, rs60288561, |
| | rs145409643, rs2380925, rs62057174, rs6565201, rs7197475, rs8048448, rs913678, rs10823062, |
| | rs116987560, rs145417449, rs10142203, rs4639966, rs503425, rs688161, rs10402677, |
| | rs696780, rs2241003, rs11130633, rs11130643, rs11915480, rs12497250, rs2176082, |
| | rs6445975, rs6445977, rs9311676, rs11655550, rs12232497, rs12938117, rs12945253, |
| | rs12946188, rs12946792, rs12947480, rs28392589, rs28535471, rs28621669, rs28648398, |
| | rs28723017, rs8079075, rs9303277, rs9907966, rs10798176, rs34884278, rs11598094, |
| | rs11539148, rs4625, rs62262116, rs79821347, rs12599402, rs12924985, rs74597067, |
| | rs6705304, rs6756736, rs16867384, rs918959, rs6131014, rs11803383, rs2688608, |
| | rs4482697, rs73440898, rs9965182, rs4850410, rs6088765, rs962885, rs10782004, |
| | rs11150608, rs11150610, rs11150612, rs11150617, rs11150619, rs1143682, rs11574630, |
| | rs11641202, rs11645917, rs11859274, rs12444713, rs12923297, rs2230429, rs2359661, |
| | rs28372932, rs36100148, rs3925074, rs3925075, rs41440449, rs4411512, rs4506917, |
| | rs9673522, rs17301013, rs12743484, rs11145763, rs11145974, rs114568253, rs2499714, |
| | rs7625006, rs11024600, rs9782955, rs114742305, rs115513376, rs142512991, |
| | rs4648892, rs2095731, rs1499077, rs463149, rs9823523, rs9855205, rs12142199, rs7325747, |
| | rs77150043, rs62131887, rs57545959, rs79558495, rs7214635, rs726848, rs41308952, |
| | rs2501800, rs12971295, rs13344313, rs425648, rs3934007, rs72799341, rs10521318, |
| | rs11117431, rs11641016, rs11649485, rs17445836, rs4388254, rs73286503, rs7726414, |
| | rs387619, rs11813373, rs1051390, rs1055382, rs1061502, rs1062099, rs10902173, |
| | rs11246189, rs11246213, rs11246217, rs1124816, rs1131665, rs11539530, rs12272314, |
| | rs12280580, rs12419694, rs12420647, rs12793222, rs12795572, rs12797496, rs12800061, |
| | rs12802200, rs12803048, rs12803132, rs12805435, rs2246614, rs2396545, rs34889521, |
| | rs35788385, rs35865896, rs36060383, rs4963127, rs4963128, rs58688157, rs6598011, |
| | rs936469 |
| Gene | SPATA2, RNF167, TTLL12, PCNXL2, SBNO2, RBMS3, TRPV2, TRIM69, UBE2R2, |
| symbols | FBRS, RNF114, HERC4, RGS9, TRIM9, TREH, CXCL17, MYCBP2, FBXW2, KCTD6, |
| | FBXL20, KLHL20, HTRA1, CDHR4, SOCS1, SOCS3, PLEKHH2, UBE2E3, UBE2C, |
| | GLMN, SYNPO2L, FBXO40, NEDD4L, HECW2, ITCH, ARHGAP27, RNF123, TRIM72, |
| | CACYBP, TRIM63, GPSM1, POMC, GRM4, GRM2, SAA1, GNG4, TAS2R1, |
| | HTR1D, ADORA3, GRM7, CCR9, TASIR3, GPR18, CCR10, SNX20, S1PR5, CXCR2 |
| | GNAI2, GNB2, CXCR1, CXCL16, CXCR5, CCR7, C5, ADCY7, FBXO42, UBB, UBA52, |
| | DRD2, FBXL19, FBXL3, FBXO31, SKP1, ABTB2, SLC15A2, UBA7, PSAP, DRD4 |

TABLE 13-2
Cluster 2 of the 67 SNP clusters.

| | |
|---|---|
| Functional | Glucocorticoid Receptor Signaling |
| Annotation | Clathrin-mediated Endocytosis Signaling |
| | Actin Nucleation by ARP-WASP Complex |
| | Regulation of Actin-based Motility by Rho |
| | Integrin Signaling |
| | Neutrophil degranulation |
| | Fc-gamma receptor signaling pathway involved in phagocytosis |
| | Neutrophil Activation via Adherence on Endothelial Cells |
| | Neutrophil Degranulation via FPR1/IL8 |
| | Leukocyte Adhesion to Endothelial Cell |
| SNPs | rs726848, rs62262116, rs12422045, rs3794060, rs4944946, rs2271293, rs4639966, |
| | rs503425, rs688161, rs74597067, rs912784, rs10101292, rs10109537, rs10481445, |
| | rs11250144, rs11783247, rs11785394, rs12677843, rs12682349, rs1435275, rs2001329, |
| | rs2043508, rs2251473, rs2409727, rs2437152, rs2572379, rs2572389, rs2572394, rs2572395, |
| | rs2572397, rs2572399, rs2736288, rs2736290, rs2736294, rs2736296, rs2736302, |
| | rs2736303, rs2736313, rs34389419, rs34801597, rs35009431, rs35223712, rs4240671, |
| | rs4631424, rs4840551, rs4841504, rs6601564, rs6601570, rs6984496, rs6985109, |
| | rs7836059, rs10048743, rs10197379, rs3768792, rs145768357, rs2389683, rs73458378, |
| | rs77966013, rs79877921, rs501089, rs513425, rs3766374, rs17617, rs4844600, rs1830847, |
| | rs4580644, rs6438700, rs1132200, rs1534154, rs2222631, rs6804441, rs1801274, rs6697139, |
| | rs10106207, rs10108347, rs10109025, rs10109167, rs1057626, rs10903340, rs11775149, |
| | rs11989439, rs12155745, rs12677146, rs12677326, rs12680762, rs13257831, rs13260727, |
| | rs13268126, rs13272061, rs13274106, rs13277113, rs1382567, rs1478892, rs1478894, |
| | rs1600249, rs1600250, rs1600252, rs17153419, rs17726209, rs17782536, rs17782554, |
| | rs17783634, rs17799486, rs17807624, rs2061831, rs2127127, rs2244894, rs2248700, |
| | rs2409718, rs2409745, rs2409749, rs2409781, rs2572369, rs2572440, rs2572446, |
| | rs2572447, rs2572448, rs2572450, rs2618473, rs2618476, rs2618479, rs2736292, |
| | rs2736305, rs2736332, rs2736340, rs2736342, rs2736346, rs2736347, rs3021494, rs3021500, |
| | rs34069332, rs35499486, rs4333549, rs4523214, rs4549730, rs4840550, rs4840567, |
| | rs4840568, rs6601561, rs6601562, rs7000141, rs7831557, rs12114284, rs1966115, |
| | rs9520836, rs12142199, rs268794, rs425648, rs1433905, rs2117209, rs12588573, rs7533588, |
| | rs10747210, rs10782004, rs11150608, rs11150610, rs11150612, rs11150615, rs11150617, |
| | rs11150619, rs1143678, rs1143682, rs1143683, rs11574630, rs11574637, rs11641202, |
| | rs11645653, rs11645917, rs11859274, rs11860650, rs12444713, rs12923297, |
| | rs13332545, rs13338069, rs2230429, rs2359661, rs28372932, rs34550882, rs34594027, |
| | rs34767000, rs36100148, rs3925074, rs3925075, rs41440449, rs41476751, rs41477949, |
| | rs4411512, rs4506917, rs4548893, rs4632147, rs72799341, rs9673522, rs9888739, |
| | rs9888879, rs9933520, rs9937837, rs16867384, rs918959, rs10911390, rs41263646, |
| | rs1143679, rs12917874, rs34082782, rs34572943, rs35314490, rs35472514, rs36001237, |
| | rs7540556, rs7197475, rs17610618, rs4666298, rs6756819, rs511580, rs10197010, |
| | rs11571293, rs10988542, rs266433, rs3864261, rs7214635, rs6945400, rs928596, |
| | rs10798176, rs1689510, rs2456973, rs2083146, rs11756587, rs1167796, rs1167797, rs6964720, |
| | rs2732159, rs3733345, rs3914167, rs10800309, rs10800314, rs116660017, rs12129787, |
| | rs12139150, rs6671847, rs771459, rs1441904, rs3769503, rs209665, rs209677, rs2244364, |
| | rs10911363, rs12094036, rs12122217, rs12753665, rs34459587, rs35423398, |
| | rs35667872, rs113506094, rs1559758, rs2553772, rs2732549, rs2785197, rs387619, rs507230, |
| | rs56228409, rs17026452, rs57545959 |
| Gene | KRTAP92, KRTAP171, P4HTM, KRT25, KRT28, KRT24, KRT16, KRT27, KRT26, KRT31, |
| symbols | KRT38, KRT13, KRT37, KRT23, KRT33A, KRT12, KRTAP59, KRT40, TSNAXIP1, |
| | UPK2, MEOX1, LRCH1, SOX7, IKZF2, ITGBL1, FAP, RGMA, LAYN, KRT20, |
| | SLAMF7, CR1, CD1B, CD38, KRT15, KRT19, THY1, CD86, CD80, KRT17, KRT14, |
| | FCGR2B, MTMR9, IL7, TNFSF13B, TNFRSF18, TMEM171, ARRDC2, OR52R1, OR11H4, |
| | OR6S1, CDC42BPA, ITGAX, ITGA4, APOBEC4, ITGAM, SELL, ITGAL, PTPRC, |
| | SDC1, CR2, CD5, CTLA4, KRT9, FNBP1, FCHO1, FCHO2, ARRB2, COBL, SYT11, |
| | DNM3, RAB5B, AMPH, SYNJ2, HIP1, SH3GL3, GAK, VAMP2, DAB2, FCGR2A, |
| | FNBP1L, SCARB2, NCK2, ACTL10, ACTR2, ARPC5, WASL, TMEM55B, CD44, |
| | RAB5C, VAMP8, ARPC2 |

TABLE 13-3
Cluster 3 of the 67 SNP clusters.

| | |
|---|---|
| Functional | Th1 and Th2 Activation Pathway |
| Annotation | Th17 Activation Pathway |
| | T Cell Exhaustion Signaling Pathway |
| | IL-23 Signaling Pathway |
| | Role of JAK1 and JAK3 in γc Cytokine Signaling |
| | interleukin-21-mediated signaling pathway |
| | AHR Signaling in Tr1 Cells Function |
| | JAK-STAT signaling pathway |
| | Interleukin-6 signaling pathway |
| | Lymphocyte Mediated Myocardial Injury in Myocarditis |
| SNPs | rs10466455, rs12150495, rs1340237, rs11804305, rs501192, rs1132200, rs56886418, |
| | rs6568431, rs6923608, rs72941674, rs742108, rs45444697, rs928596, rs10521318, |
| | rs11117431, rs11641016, rs11649485, rs17445836, rs2287339, rs75096128, rs425648, |
| | rs1464776, rs10168266, rs10174238, rs10181656, rs10931481, rs11686127, rs11889341, |
| | rs11893432, rs12463658, rs12612769, rs12990918, rs12998748, rs13010752, rs13011805, |
| | rs13017460, rs13389408, rs13401064, rs13407419, rs13426947, rs1400656, rs1517352, |
| | rs16833214, rs16833215, rs16833239, rs16833249, rs16833260, rs3024847, rs3024851, |
| | rs3024861, rs3024866, rs3024877, rs3024886, rs3024896, rs34675442, rs34946552, |
| | rs3771327, rs3821236, rs6715106, rs6736175, rs7568275, rs7574865, rs7582694, |
| | rs7594501, rs7601754, rs932169, rs907715 |
| Gene | CNP, CHIT1, IL6R, CARD17, C9orf156, TMEM39A, PRDM1, GBA, IRF8, ASB3, JAK3, |
| symbols | TFEC, STAT4, IL21 |

TABLE 13-4
Cluster 4 of the 67 SNP clusters.

| | |
|---|---|
| Functional | EIF2 Signaling |
| Annotation | Spliceosomal Cycle |
| | Regulation of eIF4 and p70S6K Signaling |
| | Coronavirus Pathogenesis Pathway |
| | mTOR Signaling |
| | Intracellular ribonucleoprotein complex |
| | nuclear-transcribed mRNA catabolic process, nonsense-mediated decay |
| | gene expression |
| | mRNA splicing, via spliceosome |
| | RNA binding |
| SNPs | rs4719714, rs73683966, rs11171, rs74597067, rs12743484, rs6705628, rs238224, |
| | rs115765118, rs928596, rs1052690, rs7939069, rs968567, rs34099611, rs12145347, |
| | rs11788118, rs1053093, rs11065513, rs111476894, rs112430375, rs113392673, rs113419431, |
| | rs113779281, rs113870709, rs141868266, rs186479757, rs74392337, rs74649380, |
| | rs75092674, rs75102897, rs75695234, rs76238820, rs77825805, rs78573504, rs78609563, |
| | rs79294954, rs80247019, rs80313423, rs6492983, rs6538678, rs10797888, rs10911342, |
| | rs10911352, rs10911363, rs10911365, rs10911366, rs11579965, rs11580439, rs11802011, |
| | rs12057355, rs12066019, rs12076338, rs12081719, rs12086699, rs12091406, |
| | rs12094036, rs12096687, rs12096702, rs12122217, rs12753665, rs17849501, rs2274065, |
| | rs2298083, rs2333681, rs34110768, rs34459587, rs35423398, rs35667872, rs3845460, |
| | rs41263646, rs77960299, rs61814766, rs4388254, rs7726414, rs12801636, rs2667978, |
| | rs2953898, rs7829816, rs7197475, rs2303108, rs7248181, rs73161005, rs9303277, |
| | rs7606861, rs7252175, rs55873962, rs2675954, rs816685, rs958476, rs1582418, rs2431098, |
| | rs2431099, rs2431697, rs10798176, rs1234314, rs1234315, rs34884278, rs138801602, |
| | rs706778, rs7172677, rs17301013, rs16837131, rs2019097, rs77528423, rs9353321, |
| | rs73286503, rs1429411, rs3731570, rs6738825, rs4639966, rs688161, rs10419198, rs11705183, |
| | rs1496453, rs11791699, rs11792269, rs1194, rs6920432, rs6938946, rs820077, |
| | rs140870671, rs112058316, rs12470957, rs17026452, rs9398235, rs11755393, rs13205210, |
| | rs13207858, rs1535001, rs205284, rs2764208, rs34840245, rs3734266, rs7764921, |
| | rs9462015, rs9462027, rs6946131, rs146696797, rs2499714, rs4917385, rs12150495, |
| | rs56228409, rs352834, rs2600669, rs62149377, rs2455331, rs2380925, rs223881, rs223883, |
| | rs13252899, rs7712962, rs79558495, rs73966422, rs11073328, rs6477694, rs930297, |
| | rs959260, rs9894206, rs7186852, rs1689510, rs2456973, rs2287339 |
| Gene | STEAP1B, SLC12A9, PLEKHH3, PAQR7, TPRKB, SCIMP, KCNC4, SSR2, IDNK, FNBP4, |
| symbols | TMEM258, NRBP2, PLEKHA6, NR4A3, MOBIA, MOB4, COLGALT2, UBQLN1, |
| | OASL, INSM2, PCNXL3, EXD1, SNRPF, SMG7, SMG5, DDX46, RPL27, PUF60, |
| | SF3B2, RPS20, CD2BP2, DHX34, SF3A1, CASC3, KCNS3, SNRPD2, DUSP11, UBBP4, |
| | NGEF, ERCC6L2, BARX2, SLU7, TNFSF4, NCBP2, UBQLN4, RBM17, RPL4, |
| | RPL19, MRPS14, ZMAT5, PPIL3, LYN, PPP1R9A, SNX14, PPP2CA, SF3B1, RPS25, |
| | RPS11, RPL3, GZMA, RAD23B, RPL10A, RPSA, USP39, CDC40, SKIV2L2, SNRPC, |
| | SEC61G, RPS10, RPEL1, STAT5, PTGES3L, PPIL6, AHSA2, RPL23A, AHRR, SRRT, |
| | NUDT2, POLR2C, CPSF1, THG1L, ZAN, LONRF2, FAM98B, FRRS1L, FASLG, MRPS7, |
| | ZNF48, UBL7, RPS26, CHAC2 |

TABLE 13-5
Cluster 5 of the 67 SNP clusters.

| | |
|---|---|
| Functional | Coagulation System |
| Annotation | Extrinsic Prothrombin Activation Pathway |
| | Intrinsic Prothrombin Activation Pathway |
| | dsDNA Break Repair by Homologous Recombination |
| | Th1 and Th2 Activation Pathway |
| | cellular protein metabolic process |
| | Ischemic stroke |
| | Lupus Erythematosus, Systemic |
| | low-density lipoprotein particle receptor binding |
| | Coronary artery disease (MI, PTCA, CABG, angina or CIHD) |
| SNPs | rs1913517, rs7094610, rs6565201, rs7186852, rs7197475, rs72799341, rs8048448, |
| | rs35355603, rs10798176, rs34884278, rs56272032, rs1689510, rs2456973, rs10521318, |
| | rs11117431, rs11641016, rs11649485, rs17445836, rs16979595, rs11681718, rs2075184, |
| | rs497273, rs76114932, rs77850688, rs1876680, rs223883, rs6538678, rs113815962, |
| | rs4242897, rs7258015, rs4149228, rs74597067, rs10254963, rs1077481, rs1077482, |
| | rs11768806, rs11972310, rs11972719, rs12706862, rs13222807, rs13222837, rs13225818, |
| | rs13227095, rs13237475, rs13240595, rs13244090, rs17340646, rs34673027, rs35939202, |
| | rs4728142, rs4728152, rs4731532, rs4731548, rs4731549, rs6467228, rs6467229, |
| | rs10402677, rs2277802, rs1351161, rs7683537, rs6492983, rs1059312, rs10773578, |
| | rs10847691, rs10847697, rs11059910, rs11059911, rs11059917, rs11059919, rs11059923, |
| | rs11059926, rs11059927, rs11059928, rs11059930, rs12298615, rs12321679, rs1385374, |
| | rs149495769, rs2170988, rs34888374, rs6486730, rs11887156, rs4369667, rs17301013, |
| | rs10499197, rs17781283, rs58721818, rs670369, rs6932056, rs12992815, rs55634455, |
| | rs1582418, rs2431098, rs2431099, rs2431697, rs4921283, rs80179578, rs989767, |
| | rs111758635, rs113205188, rs12470957, rs138408824, rs138770919, rs139376595, |
| | rs140976895, rs142176736, rs145595328, rs146366492, rs147355425, rs149756664, |
| | rs17026452, rs182643381, rs12142199, rs4363498, rs7540556, rs2380925, rs10494562, |
| | rs10752911, rs10797880, rs10797888, rs10911342, rs10911352, rs11577151, rs11802011, |
| | rs11805957, rs12045638, rs12410472, rs12565776, rs140461587, rs16860537, rs16860810, |
| | rs2298083, rs3845460, rs41263646, rs525410, rs77960299, rs4234726, rs117174224, |
| | rs2289583, rs7172677, rs10732993, rs12752692, rs1342033, rs1342034, rs1935327, |
| | rs2039983, rs2185702, rs4916342, rs6425223, rs2688608, rs2786111, rs3739817, |
| | rs10118244, rs4927193, rs2723196, rs7241191, rs11185603, rs4917014, rs2229524, |
| | rs9353321, rs662799, rs6088765, rs2303745, rs6133922, rs4474733, rs726848, rs10747210, |
| | rs10782004, rs11150608, rs11150610, rs11150612, rs11150617, rs11150619, rs1143682, |
| | rs11574630, rs11641202, rs11645653, rs11645917, rs11859274, rs12444713, rs12923297, |
| | rs2230429, rs2359661, rs28372932, rs34594027, rs36100148, rs3925074, rs3925075, |
| | rs41440449, rs41477949, rs4411512, rs4506917, rs9673522, rs57545959 |
| Gene | LRRC18, PHKG2, FAT4, ZBTB37, RARRES2, IKZF4, ZDHHC7, CLPTM1, SLC9A4, |
| symbols | SLC9A, MLEC, CD4, GZMK, CCL22, NTN4, LGALS9, CLEC4D, ICAM3, EPHX1, |
| | CDK5RAP3, TSPAN33, CEACAMITMPRSS3, CD276, C10orf54, CENPU, GINS2, |
| | OIP5, SLC15A4, TMEM163, HS3ST4, RC3H1, IFNGR1, B7RP1, MCM6, PTTG1, NUSAP1, |
| | ILIRL1, MALRD1, GGCX, VWA1, ITIH2, MFI2, CALU, F5, PRSS3, LAMC1, WFS1, |
| | APLP2, NEIL1, RAD51, TMEM63A, ANKRD65, SERPINC1, PLAU, F13B, ENG, |
| | TNFSF8, PCSK9, PSD4, TK1, CD226, IKZF1, NT5E, APOA5, ACSS2, MXRA8, ANKLE1, |
| | SNAP25, ABHD8, APOE, BRCA1, TOP2A, VKORC1, TMBIM1 |

TABLE 13-6
Cluster 6 of the 67 SNP clusters.

| | |
|---|---|
| Functional | N/A |
| Annotation | |
| SNPs | rs4845473, rs6663448, rs2304087, rs2228054, rs10747210, rs10782004, rs11150608, |
| | rs11150610, rs11150612, rs11150617, rs11150619, rs1143682, rs11574630, rs11641202, |
| | rs11645653, rs11645917, rs11859274, rs12444713, rs12923297, rs2230429, rs2359661, |
| | rs28372932, rs34572943, rs36100148, rs3925074, rs3925075, rs41440449, rs4411512, |
| | rs4506917, rs72799341, rs9673522, rs11580078, rs2241003, rs111469562, rs112345383, |
| | rs112743130, rs112876941, rs112880843, rs113115305, rs113233720, rs113370572, |
| | rs11655550, rs12232497, rs12938117, rs12945253, rs12946188, rs12946792, |
| | rs12947480, rs1453560, rs28392589, rs28535471, rs28621669, rs28648398, rs28723017, |
| | rs2941509, rs4252665, rs72832915, rs8079075, rs9303277, rs9907966, rs4810485, |
| | rs6131014, rs12722558, rs706778, rs3024493, rs3024505, rs4719714, rs73683966 |
| Gene | LCE1C, LCE3A, SPRR2F, SPRR2D, SPRR2B, LCE2A, LCE1E, SPRR1A, LCE1A, SPRR3, |
| symbols | LCE1D, SPRR1B, SMCP, LCE3C, LCE3D, SLC44A2, IL10RA, KPRP, LCE1F, LCE3E, |
| | PRSS53, IL23R, MEGF9, ORMDL3, CD40, IL2RA, IL10, IL6 |

TABLE 13-7
Cluster 7 of the 67 SNP clusters.

| | |
|---|---|
| Functional | N/A |
| Annotation | |
| SNPs | rs6084875, rs6131014, rs17885785, rs10254963, rs1077481, rs1077482, rs11768806, |
| | rs13222807, rs13225818, rs13237475, rs13240595, rs35939202, rs4728142, rs4731532, |
| | rs4731548, rs4731549, rs6467228, rs6467229, rs266433, rs268794, rs3864261, rs10225904, |
| | rs3801814, rs763616, rs45444697, rs4610334, rs238224, rs60288561, rs17079296, |
| | rs428073, rs904661, rs12422045, rs3794060, rs4944946, rs1167797, rs6964720, |
| | rs1410454, rs2675954, rs6492983, rs2289583, rs7172677, rs183211, rs962885, rs1024580, |
| | rs74597067, rs6574349, rs117528104, rs7252175, rs12050151, rs57545959, rs3109154, |
| | rs7725052, rs56228409, rs10902173, rs11246217, rs11246262, rs11246268, rs12280580, |
| | rs12420647, rs2246614, rs28392994, rs4963127, rs4963166, rs58688157, rs6598011, |
| | rs73161005, rs7179733, rs2271293, rs12148050, rs2015407 |
| Gene | PRNT, NCOA5, INS, IGF2, OPNISW, TNPO1, HOXA1, EFNA4, SORCS2, GP1BA, |
| symbols | CENPV, ULBP3, VSIG10, DHCR7, POM121C, CCDC171, C2orf82, CHAC1, SNUPN, |
| | LRRC37A, ARHGAP44, GPRC5C, NGB, CCER1, CRHR1, GIPR, TSHR, GPBAR1, GPR39, |
| | PTGER4, RAMP2, SCT, THOC5, CHRNA7, ACD, AMN |

TABLE 13-8
Cluster 8 of the 67 SNP clusters.

| | |
|---|---|
| Functional | Th1 and Th2 Activation Pathway |
| Annotation | STAT3 Pathway |
| | Natural Killer Cell Signaling |
| | PI3K/AKT Signaling |
| | Cardiac Hypertrophy Signaling (Enhanced) |
| | Interleukin-27-mediated signaling events |
| | NO2-dependent IL-12 pathway in NK cells |
| | Behcet Syndrome |
| | Selective expression of chemokine receptorsin T-cell polarization |
| | Chronic inflammatory diseases (pleiotropy) |
| SNPs | rs7787, rs10889681, rs10889687, rs1874791, rs3790564, rs12598357, rs12928404, |
| | rs7500321, rs1874886, rs2366643, rs2647930, rs564799, rs564976, rs11085727, rs11085735, |
| | rs118115488, rs12720356, rs2304256, rs2304257, rs2569703, rs280497, rs280519, |
| | rs34536443, rs34725611, rs62131887, rs7258015, rs11673460, rs425648, rs7731626, |
| | rs11681718, rs2075184, rs113815962, rs56114296 |
| Gene | IL17D, IL12RB2, IL27, IL12A, TYK2, IL12RB1, IL6ST, IL18R1, NOS2 |
| symbols | |

TABLE 13-9
Cluster 9 of the 67 SNP clusters.

| | |
|---|---|
| Functional | Caveolar-mediated Endocytosis Signaling |
| Annotation | Interferon Signaling |
| | Natural Killer Cell Signaling |
| | Coronavirus Pathogenesis Pathway |
| | IL-15 Production |
| | Interferon-gamma signaling pathway |
| | Dendritic Cell Activation in Systemic Lupus Erythematosis |
| | Th17-Cell Function in Systemic Lupus Erythematosus |
| | T-Cell Receptor -> STAT Signaling |
| | Th1-Cells Activation and Proliferation in Atherosclerosis |
| SNPs | rs352834, rs12801636, rs494003, rs931127, rs192726980, rs4625, rs17546779, rs4888360, |
| | rs10254963, rs1077481, rs1077482, rs11768806, rs13222807, rs13225818, rs13237475, |
| | rs13240595, rs4728142, rs4731532, rs4731548, rs4731549, rs6467228, rs11089629, |
| | rs11089637, rs131654, rs140489, rs140492, rs181362, rs181363, rs2070512, rs5754217, |
| | rs7444, rs2241003, rs116660017, rs7970893, rs2044264, rs10484433, rs13191296, |
| | rs13195509, rs13196552, rs13199775, rs13220395, rs1324087, rs1324088, rs16891235, |
| | rs16891264, rs16891315, rs16891334, rs17587226, rs17587597, rs1892250, rs1892253, |
| | rs2072806, rs3734523, rs3734528, rs3857546, rs4712969, rs6940007, rs9379858, |
| | rs9379895, rs9393708, rs9393710, rs164178, rs3766374, rs34709472, rs2407782, rs10494562, |
| | rs10752911, rs10797880, rs10797888, rs10911300, rs10911319, rs10911342, |
| | rs10911352, rs10911363, rs10911365, rs10911366, rs10911390, rs1122259, rs11577151, |
| | rs11579965, rs11580439, rs11802011, rs11805957, rs12032353, rs12045638, rs12057355, |
| | rs12066019, rs12086699, rs12091406, rs12094036, rs12096702, rs12119966, |
| | rs12122217, rs12409487, rs12410472, rs12753665, rs16860810, rs17849501, rs2022013, |
| | rs2078087, rs2274065, rs2298083, rs2333681, rs34110768, rs34459587, rs35423398, |
| | rs35667872, rs3845460, rs41263646, rs77960299, rs12248123, rs74623793, rs4971066, |
| | rs8105429, rs9303891, rs9887904, rs913678, rs704853, rs10101292, rs10106207, rs10108347, |
| | rs10109025, rs10109167, rs10109537, rs10481445, rs1057626, rs10903340, |
| | rs11250144, rs11775149, rs11783247, rs11785394, rs11989439, rs12155745, rs12677146, |
| | rs12677326, rs12677843, rs12680762, rs12682349, rs13257831, rs13260727, rs13268126, |
| | rs13272061, rs13274106, rs13277113, rs1382567, rs1435275, rs1478892, rs1478894, |
| | rs1478897, rs1564267, rs1600249, rs1600250, rs1600252, rs17153419, rs17726209, |
| | rs17782536, rs17782554, rs17783634, rs17799486, rs17807624, rs2001329, rs2043508, |
| | rs2061831, rs2127127, rs2244894, rs2248696, rs2248700, rs2248932, rs2251473, |
| | rs2254546, rs2280805, rs2409718, rs2409727, rs2409745, rs2409749, rs2409781, rs2437152, |
| | rs2572369, rs2572379, rs2572389, rs2572394, rs2572395, rs2572397, rs2572399, |
| | rs2572440, rs2572446, rs2572447, rs2572448, rs2572450, rs2618458, rs2618473, rs2618476, |
| | rs2618479, rs2736288, rs2736290, rs2736292, rs2736294, rs2736296, rs2736302, |
| | rs2736303, rs2736305, rs2736313, rs2736332, rs2736340, rs2736342, rs2736345, |
| | rs2736346, rs2736347, rs2736350, rs3021494, rs3021500, rs34069332, rs34389419, rs34801597, |
| | rs35009431, rs35223712, rs35499486, rs4240671, rs4333549, rs4523214, rs4549730, |
| | rs4631424, rs4840550, rs4840551, rs4840567, rs4840568, rs4841504, rs6601561, |
| | rs6601562, rs6601564, rs6601570, rs6984496, rs6985109, rs7000141, rs7812879, rs7820492, |
| | rs7836059, rs11539148, rs13023380, rs1990760, rs2389683, rs35667974, rs501192, |
| | rs2478118, rs10271373, rs12743484, rs1378942, rs2289583, rs7172677, rs113815962, |
| | rs56114296, rs10857650, rs11101442, rs1913517, rs2663052, rs2663054, rs2928402, |
| | rs2928403, rs2940707, rs7094610, rs7097397, rs7922169, rs877819, rs10156169, |
| | rs10229001, rs10236569, rs10239340, rs10279821, rs10488631, rs1072767, rs10954213, |
| | rs10954214, rs113253472, rs11761199, rs11763959, rs11767954, rs11972719, rs12155080, |
| | rs12531054, rs12531711, rs12535158, rs12536266, rs12537264, rs12537284, |
| | rs12539476, rs12539741, rs12706860, rs12706861, rs12706862, rs13222837, rs13227095, |
| | rs13236009, rs13239597, rs13244090, rs17339836, rs17340351, rs17340646, rs17424602, |
| | rs17425212, rs2070197, rs2242028, rs2272347, rs2280714, rs34673027, rs34725944, |
| | rs34815476, rs35000415, rs35188261, rs35234849, rs35939202, rs3757385, rs3757387, |
| | rs3778750, rs3778753, rs3807134, rs3807306, rs3807307, rs3847099, rs4728144, |
| | rs4731530, rs4731531, rs4731533, rs4731536, rs4731540, rs4731541, rs59110799, rs6467229, |
| | rs729302, rs7808907, rs16840423, rs183211, rs1061511, rs72739935, rs562954, |
| | rs117682184, rs62262116, rs56228409, rs4639966, rs17805636, rs724710, rs1887428, |
| | rs36051895, rs7584501, rs11640251, rs137870785, rs17617, rs2244364, rs9603612, |
| | rs11865121, rs12599402, rs12708715, rs12924729, rs12924985, rs12925642, rs12928537, |
| | rs2041670, rs2058531, rs3901386, rs6498166, rs7200786, rs9652601, rs62097857, |
| | rs928596, rs4644492, rs12753920, rs72952888, rs3925535, rs7311756, rs12232497, rs4252665, |
| | rs72832915, rs9303277, rs9651118, rs10466455 |
| Gene | NRD1, MICAL1, RNASEH2C, IP6K2, KCNK2, SGK223, CTRB1, FAM71F2, |
| symbols | ENSG00000239665, PPIL2, RAB14, FCER1G, OAS1, GBP3, TRIM38, OAS3, SH2D1B, |
| | CD244, LILRB2IRAK4, NCF2, LRRC16A, TRIM8, CADPS2, KCNN3, GDF15, THOC1, |
| | IFI16, PTPN1, CD247, FAM167A, ATRIP, IFIH1, CASP1, PRPF18, KIAA1549, |
| | CNKSR1, ZC3HAV1, CSK, KSR1, WDFY4, IRF5, SPTA1, NSF, COPA, NAV1, KCNB1, |
| | KCND2, TMEM115, CNTNAP1, ARCN1, ANK3, BCL2L11, JAK2, ALK, COG4, COG5, |
| | CD55, CEP68, COG6, CLEC16A, PTPN2, RAB1A, ARHGEF2, CAPZA1, EPHX4, |
| | SLC2A13, ERBB2, CLCN6, EHF |

TABLE 13-10
Cluster 10 of the 67 SNP clusters.

| | |
|---|---|
| Functional | Creatine-phosphate Biosynthesis |
| Annotation | Trans, trans-farnesyl Diphosphate Biosynthesis |
| | Superpathway of Geranylgeranyldiphosphate Biosynthesis I (via Mevalonate) |
| | Role of Lipids/Lipid Rafts in the Pathogenesis of Influenza |
| | Bupropion Degradation |
| | regulation of keratinocyte migration |
| | transmembrane receptor protein phosphatase activity |
| | negative regulation of epithelial cell migration |
| | Metabolism of Estrogens and Androgens |
| | Oxidative Stress Protection via AHR-NFE2L2-NQO1 Signaling in Keratinocytes |
| SNPs | rs2726101, rs169080, rs967616, rs4474733, rs74597067, rs9651118, rs1051390, |
| | rs1055382, rs1061502, rs1062099, rs10902173, rs11246189, rs11246213, rs11246217, |
| | rs11246262, rs11246268, rs1124816, rs1131665, rs11539530, rs12272314, rs12280580, |
| | rs12419694, rs12793222, rs12795572, rs12797496, rs12800061, rs12802200, rs12803048, |
| | rs12803132, rs12805435, rs2246614, rs2396545, rs28392994, rs34889521, rs35788385, |
| | rs35865896, rs36060383, rs4963127, rs4963166, rs58688157, rs936469, rs34099611, |
| | rs4639966, rs6492983, rs364418, rs631574, rs10466455, rs10742326, rs74068700, |
| | rs10106207, rs10108347, rs10109167, rs10481445, rs10903340, rs11250144, rs11775149, |
| | rs11989439, rs12155745, rs12680762, rs13257831, rs13272061, rs1382567, rs1478894, |
| | rs1600250, rs1600252, rs17726209, rs17782536, rs17782554, rs17799486, rs17807624, |
| | rs2127127, rs2244894, rs2251473, rs2409718, rs2409745, rs2409749, rs2572397, |
| | rs2736342, rs3021500, rs34801597, rs4840550, rs4840551, rs4840567, rs4841504, |
| | rs6601570, rs7836059, rs12096737, rs6700189, rs12588573, rs116101946, rs1051624, |
| | rs111744793, rs139278659, rs2271293, rs209665, rs209677, rs4644492, rs4942903, |
| | rs9568353, rs2304087, rs1131265, rs1132200, rs2222631, rs8105429, rs6574349, rs45444697, |
| | rs4971066, rs928596, rs4648892, rs114802133, rs6565201, rs7186852, rs7197475, rs1378942 |
| Gene | EEPD1, KDM4B, GALNTL6, PTRF, PLOD1, CDHR5, EPPK1, TMPRSS4, SPINT1, |
| symbols | PTPRM, TRIM44, PTPRU, USP17L2, LIN9, METTL3, MAP4K4, CDH17, PRMT7, |
| | CBFA2T2, ST7L, TRIM13, C19orf38, ARHGAP31, PGPEP1, ANGEL1, THBS3, E2F2, |
| | FDPS, IQGAP2, SRCAP, CYP1A1 |

TABLE 13-11
Cluster 11 of the 67 SNP clusters.

| | |
|---|---|
| Functional | N/A |
| Annotation | |
| SNPs | rs6574349, rs6088765, rs57545959, rs1052690, rs74597067, rs13168551, rs10521318, |
| | rs11117431, rs11641016, rs11649485, rs17445836, rs501089, rs513425, rs7172677, |
| | rs209665, rs209677, rs10800309, rs12139150, rs6671847, rs117830658, rs16837131 |
| Gene | IRF2BPL, PROCR, PNKD, FRMD3, KCTD2, SLC36A1, COX4I1, SDHD, COX5A, |
| symbols | E2F1, UQCC1, SDHC, HIBADH, NDUFB3 |

TABLE 13-12
Cluster 12 of the 67 SNP clusters.

| | |
|---|---|
| Functional | Death Receptor Signaling |
| Annotation | Induction of Apoptosis by HIV1 |
| | Endoplasmic Reticulum Stress Pathway |
| | Leukocyte Extravasation Signaling |
| | Cardiac Hypertrophy Signaling (Enhanced) |
| | Apoptosis |
| | MAPK Signaling Pathway |
| | Fas signaling pathway |
| | Granzyme A-mediated apoptosis pathway |
| | Lipid and atherosclerosis |
| SNPs | rs79404002, rs73161005, rs6088765, rs428073, rs904661, rs62057174, rs6565201, |
| | rs7186852, rs7197475, rs72799341, rs8048448, rs7764323, rs916287, rs12588573, rs1170436, |
| | rs2271293, rs4783754, rs3733345, rs2055802, rs7692950, rs4355385, rs61794854, |
| | rs3024505, rs2732159, rs10774625, rs17630235, rs597808, rs653178, rs144526725, |
| | rs74597067, rs2431697, rs10507521, rs11842000, rs17065161, rs17462017, rs17571812, |
| | rs17653353, rs2121037, rs2325088, rs4942249, rs61209988, rs61959960, rs61959998, |
| | rs7989702, rs7995955, rs4963128, rs2288012, rs189257248, rs6131014, rs501089, |
| | rs513425, rs6705628, rs930297, rs959260, rs9894206, rs16837131, rs9651118, rs6814866, |
| | rs73223282, rs73223294, rs116660017, rs340630, rs4283545, rs2928402, rs2928403, |
| | rs12822507, rs3914167, rs230261, rs34006827, rs11539148, rs4625, rs62262116, |
| | rs79821347, rs6568431, rs12664535, rs12129787, rs62131887, rs7172677, rs45444697, |
| | rs4971066, rs928596, rs10878246, rs17466626, rs3925535, rs7311756, rs2541117, rs11741255, |
| | rs4639966, rs9398235, rs111737889, rs12108299, rs12500106, rs12500188, rs12501889, |
| | rs12503080, rs12504328, rs12506429, rs12506703, rs12509044, rs12509545, |
| | rs12512006, rs148274675, rs149586506, rs16842284, rs73209462, rs73209465, rs73209470, |
| | rs73211351, rs73211357, rs73211366, rs73211381, rs73211383, rs73211388, |
| | rs73211394, rs73211401, rs73213404, rs73213405, rs73213406, rs73213408, rs73213413, |
| | rs73223242, rs73223246, rs73223250, rs76852463, rs77743832, rs497273, rs2425223, |
| | rs1922240 |
| Gene | RPAIN, MTFP1, TP53INP2, TAOK3, PRR14, ETV7, ENSG00000125954, ZNF219, |
| symbols | NFATC3, TMEM175, TEC, CLNK, FAIM3, CPEB1, MAPKAPK5, RFX2, TRAF4, CCNJL, |
| | CCDC122, PIDD1, LACC1, GINS3, DFFB, ELMO2, HSPB2, HTRA2, SLC25A19, |
| | CASP10, MFN2, MAP1LC3A, GPR78, ATF6, MAPK10, IL17RD, ARHGAP22, MMP24, |
| | CREBL2, C9, NPHS1, PARP2, MST1R, LIN28B, BAG2, HSPA6, CDC37, SNX33, |
| | ADAM15, NCKIPSD, LRRK2, ACTG2, ETV6, RAPGEF6, CCDC84, DDO, ACOX3, |
| | ACADS, MYL9, APEX, ABCB1, RIT1 |

TABLE 13-13
Cluster 13 of the 67 SNP clusters.

| | |
|---|---|
| Functional | Breast Cancer Regulation by Stathmin1 |
| Annotation | CREB Signaling in Neurons |
| | Dopamine-DARPP32 Feedback in cAMP Signaling |
| | Synaptic Long-Term Potentiation |
| | P2Y Purigenic Receptor Signaling Pathway |
| | X-linked severe congenital neutropenia |
| | X-linked thrombocytopenia with normal platelets |
| | Vascular Endothelial Cell Vasodilation in Brain |
| | regulation of cardiac muscle contraction |
| | CR3 Mediated Phagocytosis in Neutrophils and Macrophages |
| SNPs | rs726848, rs74597067, rs645078, rs602662, rs632111, rs12588573, rs11788947, rs501192, |
| | rs71606243, rs12598357, rs12928404, rs7500321, rs9428886, rs3783759, rs7186852, |
| | rs7197475, rs3862550, rs7900818, rs6663448, rs10494562, rs10752911, rs10797880, |
| | rs10797888, rs10911300, rs10911319, rs10911342, rs10911352, rs1122259, rs11577151, |
| | rs11802011, rs11805957, rs12032353, rs12045638, rs12119966, rs12409487, |
| | rs12410472, rs12565776, rs16860537, rs16860810, rs2022013, rs2078087, rs2298083, |
| | rs3845460, rs41263646, rs525410, rs77960299, rs7982251, rs7982957, rs1016350, rs55869446, |
| | rs74974648, rs76392962, rs73161005, rs12312476, rs138384991, rs139539455, |
| | rs140351402, rs140557893, rs144231913, rs149214948, rs150256879, rs41452648, |
| | rs41478451, rs78374842, rs79836876, rs9658386, rs9658397, rs9658480, rs9658527, |
| | rs9658550, rs9658574, rs150389346, rs742108, rs2235947, rs7264711, rs1048257, rs454425, |
| | rs1550250, rs11235715, rs497273, rs10521318, rs11117431, rs11641016, rs11649485, |
| | rs17445836, rs912784, rs962885, rs10402677, rs117301471, rs145977671 |
| Gene | KRTAP15, GEMIN4, PPP1R14, SPHK2, PITPNC1, TEP1, DOCK8, CARD16, TRPC3, |
| symbols | NPIPB9, WDR64, PRKCH, TMEM219, C16orf54, C16orf92, MAZ, NRG3, S100A6, |
| | LAMC2, FLT1, PLEK, SMYD3, MTMR3, PLCB3, NOS1, PREP, PLCB1, GPR132, GPR65, |
| | KIDINS220, P2RY6, P2RX4, COTL1, CORO1A, LCP1, FMNL1, LIPE, ADRA1A, |
| | PRKCA |

TABLE 13-14
Cluster 14 of the 67 SNP clusters.

| | |
|---|---|
| Functional | N/A |
| Annotation | |
| SNPs | rs10916668, rs12408855, rs11235715, rs6492983, rs11070264, rs882829, rs12743484, |
| | rs752010, rs6088765, rs116416688, rs2055802, rs7692950, rs7172677, rs2304087, |
| | rs2271293, rs2241003, rs425648, rs116660017, rs17079296, rs9861188, rs10857712, |
| | rs4708114, rs1496453, rs3785437, rs238224, rs12141391, rs150568573, rs1441904, rs9783215, |
| | rs35853026, rs71306055, rs930297, rs959260, rs9894206, rs633040, rs12666575, |
| | rs2836882, rs1990313, rs9303891, rs34330, rs2688608, rs11539148, rs62282423, |
| | rs62282430, rs726848, rs79404002, rs4639966, rs1012894, rs1018552, rs1040893, rs11152964, |
| | rs12526490, rs2065082, rs2179175, rs2299864, rs3827644, rs548234, rs56886418, |
| | rs573775, rs6568431, rs6568432, rs6923608, rs72941674, rs742108, rs9373839, rs9399975, |
| | rs12142199, rs12917712, rs73161005, rs12248123, rs1292043, rs1804182, rs4773785, |
| | rs9524400, rs34099611, rs16979595, rs12598357, rs12928404, rs141162791, |
| | rs150739866, rs13000817, rs10933559, rs13026988, rs147622113, rs12664535, rs113386660, |
| | rs6705628, rs4902562, rs11865121, rs12599402, rs12708715, rs12924729, rs12924985, |
| | rs12925642, rs12928537, rs2041670, rs2058531, rs243323, rs6498166, rs7200786, |
| | rs78318981, rs8061370, rs9652601, rs11951576, rs223881, rs223883, rs79074797, |
| | rs74597067, rs45444697, rs928596, rs11185603, rs4917014, rs6920220, rs6913643, rs6695567, |
| | rs503425, rs688161, rs145691683, rs6601327, rs1463485, rs11130633, rs11130643, |
| | rs1127745, rs11915480, rs12497250, rs6445975, rs6445977, rs9311676, rs4388254, |
| | rs7726414, rs6073958, rs6131014, rs7679, rs11057864, rs17413405, rs17483887 |
| Gene | OTUD3, PAAF1, TYRO3, KNSTRN, SLC30A2, HIVEP3, FAM83C, DCLK3, TXK, |
| symbols | ZWILCH, SPC24, PSKH1PSMB10, PSMD5, IFI30, FCGR3B, ULBP2, LSAMP, SPRN, |
| | NRN1L, CD109, CDC20B, CDK3, MIS12NEGR1, FAM47E, CELF2, CTRL, NUP210, |
| | NUP85, NTM, STMN1, MAD1L1, PSMG1, RAD51AP1, USP14CDKN1B, ZSWIM8, |
| | CDC25A, TOPBP1, CDC6, PSMB6, ATP5L, ATP5C1, ATG5, ATAD3C, CDIPTNIPSNAP1, |
| | NDST2, SUFU, SKA2, PLAT, GPC6, C14orf6, PVRL2GPIHBP1, APOC2APOC1, |
| | APOC4, APOBR, HPR, PCYOX1, HDLBP, ILF3, PRIM2, SPDL1, COA3, MTHFD2, |
| | DCTN1, RAD51B, RMI2, NME6, PAPD7, COQ9, REV3L, ATP5H, PKLR, SLC25A11, |
| | DLD, PEX7, PSMB1, SCP2, H2AFX, STAG1, TNKS, ACOX1, ACOX2, NDUFS2, |
| | VDAC1, PLTP, LCAT, SCARB1, ABCG4, CHEK2 |

TABLE 13-15
Cluster 15 of the 67 SNP clusters.

| | |
|---|---|
| Functional | Keratin filament |
| Annotation | Intermediate filament |
| | Eukaryotic transcription initiation |
| | channel activity |
| SNPs | rs12422045, rs3794060, rs4944946, rs74597067, rs726848, rs2298455, rs7566538 |
| Gene | KRTAP5, 8, SLC35G6, KRTAP161, KRTAP57, KRTAP23, KRTAP24, KRTAP412, |
| symbols | KRTAP99, KRTAP4-6, KRTAP9-6, RNF121, KRT222, KRTAP3-1, KRTAP5-10, |
| | AQP12B |

TABLE 13-16
Cluster 16 of the 67 SNP clusters.

| | |
|---|---|
| Functional | N/A |
| Annotation | |
| SNPs | rs11089629, rs11089637, rs12158299, rs12484550, rs131665, rs140489, rs140492, |
| | rs140498, rs140499, rs181360, rs181362, rs181363, rs181366, rs2070512, rs2256609, rs2266959, |
| | rs2266961, rs2266963, rs2283790, rs2298428, rs2298429, rs3747093, rs4821108, |
| | rs4821112, rs4821116, rs4821124, rs5754217, rs5754467, rs5998619, rs73166632, rs7444, |
| | rs912784, rs11130633, rs11130643, rs11915480, rs12497250, rs6445975, rs6445977, |
| | rs9311676, rs4644492, rs11640251, rs7411387, rs602097, rs11738358, rs26657, |
| | rs4869313, rs11612569, rs913678, rs7214635, rs131654, rs2283789, rs463426, rs738129, |
| | rs10101292, rs10106207, rs10108347, rs10109025, rs10109167, rs1057626, rs11783247, |
| | rs11989439, rs1564267, rs17726209, rs17782536, rs17782554, rs17783634, rs17799486, |
| | rs2001329, rs2061831, rs2254546, rs2409718, rs2409727, rs2736332, rs28633434, |
| | rs3021494, rs3021500, rs34069332, rs35223712, rs4240671, rs4333549, rs4523214, |
| | rs4840550, rs4840551, rs4841504, rs6601561, rs6601562, rs6601564, rs6984496, rs6985109, |
| | rs7000141, rs7812879, rs10036748, rs10057690, rs13160369, rs13168551, rs1422673, |
| | rs1559127, rs17111708, rs2042234, rs3792782, rs3792784, rs3792785, rs4958881, |
| | rs4958882, rs6889239, rs73272828, rs73272842, rs7708392, rs960709, rs1049564, |
| | rs12588573, rs10798176, rs34884278, rs4971066, rs928596, rs16837131, rs2019097, rs74597067, |
| | rs10013187, rs10016018, rs10028805, rs10031210, rs10446682, rs10446708, |
| | rs10516486, rs10516487, rs11097761, rs1125271, rs11736568, rs11929782, rs11931087, |
| | rs11935577, rs11944577, rs11944613, rs12163856, rs12331595, rs12331849, rs12498921, |
| | rs13106926, rs13107612, rs13117264, rs13126505, rs13129744, rs13136297, rs13136796, |
| | rs13144307, rs13145909, rs13146194, rs17031641, rs17200824, rs17266357, |
| | rs17266552, rs17266594, rs2216546, rs34029191, rs34499378, rs34749007, rs35194352, |
| | rs35388091, rs35416717, rs35838403, rs3733197, rs4270588, rs4270589, rs4276281, |
| | rs4296682, rs4398527, rs4411998, rs4426778, rs4443287, rs4493533, rs4496585, rs4522865, |
| | rs4572884, rs4607219, rs4610336, rs4637409, rs4698972, rs4698985, rs4699258, |
| | rs4699261, rs4699262, rs7656720, rs6478522, rs874610, rs1051390, rs1055382, rs1061502, |
| | rs1062099, rs10902173, rs11246189, rs11246213, rs11246217, rs1124816, rs1131665, |
| | rs11539530, rs12272314, rs12280580, rs12419694, rs12420647, rs12793222, |
| | rs12795572, rs12797496, rs12800061, rs12802200, rs12803048, rs12803132, rs12805435, |
| | rs2246614, rs2396545, rs34889521, rs35788385, rs35865896, rs36060383, rs4963127, |
| | rs4963128, rs58688157, rs6598011, rs936469, rs1127745, rs180977001, rs2176082, |
| | rs11755393, rs13205210, rs13207858, rs1535001, rs205284, rs2764208, rs34840245, rs3734261, |
| | rs3734266, rs6920432, rs7764921, rs820077, rs9462015, rs9462027, rs4888360, |
| | rs145417449, rs1039917, rs2955587, rs330048, rs6601327, rs1378942, rs2289583, |
| | rs7172677, rs10499197, rs17781283, rs2230926, rs3757173, rs5029924, rs5029937, rs5029939, |
| | rs5029949, rs58721818, rs670369, rs6920220, rs6932056, rs2244364, rs268134, rs6743585, |
| | rs10109537, rs10481445, rs10903340, rs11250144, rs11775149, rs11785394, |
| | rs12155745, rs12677146, rs12677326, rs12677843, rs12680762, rs12682349, rs13257831, |
| | rs13260727, rs13268126, rs13272061, rs13274106, rs13277113, rs1382567, rs1435275, |
| | rs1478892, rs1478894, rs1478897, rs1600249, rs1600250, rs1600252, rs17153419, |
| | rs17807624, rs2043508, rs2127127, rs2244894, rs2248696, rs2248700, rs2248932, rs2251473, |
| | rs2280805, rs2409745, rs2409749, rs2409781, rs2437152, rs2572369, rs2572379, |
| | rs2572389, rs2572394, rs2572395, rs2572397, rs2572399, rs2572440, rs2572446, rs2572447, |
| | rs2572448, rs2572450, rs2618458, rs2618473, rs2618476, rs2618479, rs2736288, |
| | rs2736290, rs2736292, rs2736294, rs2736296, rs2736302, rs2736303, rs2736305, rs2736313, |
| | rs2736340, rs2736342, rs2736345, rs2736346, rs2736347, rs2736350, rs34389419, |
| | rs34801597, rs35009431, rs35499486, rs4549730, rs4631424, rs4840567, rs4840568, |
| | rs6601570, rs7820492, rs7836059, rs77150043, rs11246262, rs11246268, rs28392994, |
| | rs4963166, rs1072626 |
| Gene | YDJC, SIAH3, DNASE1L3, CTTNBP2NL, EXOSC6, CHIA, MAP3K8, ERAP1, UTP20, |
| symbols | SNAI1, PELP1, UBE2L3, XKR6, TNIP1, RNASE6, TNFSF18, MUC1, CFLAR, SUMO2, |
| | BANK1, ANP32B, PHRF1, PXK, UHRF1BP1, CASP8, BCAR1, CCNB1IP1, ERN1, |
| | ERI1, RPP25, TNFAIP3, SPRED2, BLK, PAPD5, LRRC56, MTTP, RPP14, HRAS |

TABLE 13-17
Cluster 17 of the 67 SNP clusters.

| | |
|---|---|
| Functional | N/A |
| Annotation | |
| SNPs | rs10988542, rs10889681, rs10889687, rs1874791, rs3790564, rs74597067, rs726848, |
| | rs1133950, rs8066255, rs7625006, rs4917385, rs12598357, rs12928404, rs7500321, |
| | rs11085735, rs118115488, rs12720356, rs2304256, rs2304257, rs74908652, rs11647067, |
| | rs12232497, rs9303277, rs3024505, rs4910907, rs17105765, rs12142199, rs1292043, |
| | rs428073, rs904661, rs10466455, rs10742326, rs1355223, rs1559758, rs2553772, rs387619, |
| | rs507230, rs11130633, rs11130643, rs11915480, rs12497250, rs180977001, rs2176082, |
| | rs6445975, rs6445977, rs9311676, rs2303745, rs7197475, rs6088765 |
| Gene | USP20, SERBP1, EIF5A, EIF1, EIF3H, MRPL45, HEMK1, PDCD11, EIF3C, MRPL4, |
| symbols | CIRH1A, RAVER1, USP31, PSMD3, EIF2D, RRM1, EIF4B, MRPL20, RPS6KB1, PEBP1, |
| | PDHX, TUFM, PDHB, MRPL34, MAPK3, EIF6 |

TABLE 13-18
Cluster 18 of the 67 SNP clusters.

| | |
|---|---|
| Functional | N/A |
| Annotation | |
| SNPs | rs6492983, rs74597067, rs2836882, rs1869486, rs928596, rs2303108, rs7248181, rs4648892, |
| | rs965355, rs11145974, rs11145763, rs6478522, rs874610, rs12588573, rs12971295, |
| | rs13344313, rs425648, rs7522008, rs2304087, rs11755393, rs13205210, rs13207858, |
| | rs1535001, rs2764208, rs34840245, rs3734261, rs3734266, rs7764921, rs9462015, |
| | rs9462027, rs7186852, rs7197475, rs2816317, rs12978179, rs12986413, rs11803383, |
| | rs72952888, rs181543137 |
| Gene | RPAP1, TAF15, HMGN1, ICE2, MSTO1, CPSF3L, ZC3H4, TCEA3, GTF2A2, DNLZ, |
| symbols | SNAPC4, FOXE1, SDCCAG3, SUPT16H, LRRC25, PMPCA, FUBP1, CARM1, TAF11, |
| | FANCE, PPP4C, CDC73, DOT1L, ELL, RTF1, RPAP2, ASTN2 |

TABLE 13-19
Cluster 19 of the 67 SNP clusters.

| | |
|---|---|
| Functional | Calcium Signaling |
| Annotation | Guanosine Nucleotides Degradation III |
| | Urate Biosynthesis/Inosine 5′-phosphate Degradation |
| | Adenosine Nucleotides Degradation II |
| | tRNA Splicing |
| | Adipose tissue |
| | Heart |
| | Asparagine N-linked glycosylation |
| | Cardiac muscle |
| | regulation of cytoplasmic mRNA processing body assembly |
| SNPs | rs11804305, rs11539148, rs12150495, rs726848, rs425648, rs80193959, rs6705628, |
| | rs11145763, rs11145974, rs116912920, rs12114284, rs1966115, rs6478522, rs874610, |
| | rs10481445, rs11775149, rs13272061, rs1382567, rs1478897, rs1600250, rs1600252, |
| | rs17799486, rs2127127, rs2251473, rs2572397, rs2736342, rs4840567, rs600751, rs6574349, |
| | rs34099611, rs2576837, rs10800816, rs4363498, rs4748940, rs1450712, rs454425, |
| | rs13399274, rs1039917, rs2955587, rs330048, rs6601327, rs196432, rs11681903, rs7683537, |
| | rs6492983, rs9782955, rs930297, rs10494562, rs10752911, rs10797880, rs10911300, |
| | rs10911319, rs10911363, rs1122259, rs11577151, rs11805957, rs12032353, rs12045638, |
| | rs12076338, rs12081719, rs12096687, rs12119966, rs12122217, rs12409487, |
| | rs12410472, rs12753665, rs16860810, rs17484292, rs2022013, rs2078087, rs41263646, |
| | rs10225904, rs3801814, rs763616, rs4802307, rs62136099, rs4811174, rs13272330, |
| | rs2298455, rs10747210, rs10782004, rs11150608, rs11150610, rs11150612, rs11150617, |
| | rs11150619, rs1143682, rs11574630, rs11641202, rs11645653, rs11645917, rs11859274, |
| | rs12444713, rs12923297, rs13332545, rs2230429, rs2359661, rs28372932, rs36100148, |
| | rs3925074, rs3925075, rs41440449, rs41477949, rs4411512, rs4506917, rs4548893, |
| | rs9673522, rs9937837, rs12096737, rs6700189, rs6756736, rs74597067, rs4242897, |
| | rs6663448, rs11136577, rs10762171, rs10823062, rs56228409, rs4845473, rs1378942, |
| | rs2289583, rs7172677, rs12588573, rs1170426, rs1170436, rs965355, rs4639966, rs503425, |
| | rs688161, rs11070264, rs10933559, rs13026988, rs62057174, rs6565201, rs7186852, |
| | rs7197475, rs72799341, rs8048448, rs6586128, rs7872388, rs224031, rs4707585, rs597325, |
| | rs72928038, rs139810877, rs145409643, rs10774625, rs17630235, rs597808, rs653178, |
| | rs1061511, rs17362529, rs12598357, rs12928404, rs7500321, rs9823523, rs9855205, |
| | rs4622329, rs4246905, rs1559758, rs507230, rs1332099, rs4459332, rs36001488, |
| | rs116660017, rs12248123, rs4917385, rs2277802, rs501089, rs513425, rs4690055, |
| | rs11612569, rs2256597, rs2787733, rs73366469, rs183211, rs11231814, rs4930194, rs1108131, |
| | rs11681718, rs2075184, rs1892250, rs1892253, rs4712969, rs11640251, rs10101292, |
| | rs10109025, rs11783247, rs4240671, rs6984496, rs6985109, rs7831557, rs4625, |
| | rs62262116, rs79821347, rs12992815, rs55634455, rs2688608, rs3773377, rs10506577, |
| | rs141947161, rs912784, rs1975197, rs117325149, rs4388254, rs7726414, rs11788118, |
| | rs2600669, rs62149377, rs111476894, rs111546231, rs112430375, rs113419431, rs113779281, |
| | rs113870709, rs141868266, rs186479757, rs74392337, rs74649380, rs75092674, |
| | rs75102897, rs75695234, rs76238820, rs77825805, rs78573504, rs78609563, rs79294954, |
| | rs80247019, rs80313423, rs10411749, rs959260, rs9894206, rs2275247, rs74782705, |
| | rs415407, rs424561, rs7731626, rs4796445, rs1049564, rs10254284, rs12531540, |
| | rs1635852, rs702814, rs849142, rs886663, rs10405730, rs2816317, rs6762714, rs62324212, |
| | rs907715, rs4513082, rs3780205, rs944333, rs6131014, rs7679, rs4474733, rs6449173, |
| | rs7442295, rs6088765, rs3024505, rs1509461, rs979233, rs7114131, rs6814866, |
| | rs73223282, rs73223294, rs11024600, rs1496453, rs7249065, rs118115488, rs147622113, |
| | rs280497, rs280519, rs34536443, rs238224, rs6923307, rs141162791, rs150739866, |
| | rs56262793, rs4974949, rs34675442, rs932169, rs11756587, rs11130633, rs11130643, |
| | rs11915480, rs12497250, rs180977001, rs2176082, rs6445975, rs6445977, rs9311676, |
| | rs1861525, rs57545959 |
| Gene | SHE, TMA7, KLHL10, KIAA1683, KLHL6, TMEM18, SEC16A, MPHOSPH9, |
| symbols | ZC2HC1A, JMJD2C, NANS, FAM86B1, DCPS, GSTZ1, MAPK15, PELI1, RNPEP, KIN, |
| | GALC, VRK2, PPP1R3B, RCAN3, INSIG2, PRIMPOL, CHP1, NID1, NT5C, NMNAT2, |
| | SKAP2, CCDC8, NFATC2, MMP16, IL18BP, PYDC1, H3F3A, SLC3A1, H3F3B, |
| | CLEC6A, CLEC4E, S100A12, CSMD1, CTNNA3, NT5C3B, C1orf68, LMAN1L, RNASE1, |
| | TMEM180, CDH3, BNIP2, PHLDB1, BMF, BOK, KAT8, BCKDK, PPP5C, RNLS, |
| | GLIS3, EGR2, BACH2, NUDT16, PATL2, TMEM116, CASQ1, GJA5, NFATC2IP, |
| | FYCO1DRAM1, ULK3, TNFSF15, CARD9, UVRAG, NKX23, ATG16L2, ATG16L1, |
| | DUSP12CNNM2, USMG5, C10orf32, AS3MT, UBASH3A, PPP2RIB, TNIP2, ARL1, |
| | C10orf107, GTF2I, SPPL2C, SCYL1, CAMK1D, IL18RAP, SLC17A4, SULT1A2, ATXN2L, |
| | RABEP2, ATP2A1, SH2B1, SPNS1, VAC14, MSRA, MST1, IP6K1, MAP3K19, |
| | SEC24CCHL1, CNOT2, ATXN2, CPB2, PTPRD, MGAT4C, ENSG00000086848, DPAGT1MPI, |
| | GMPPB, CDKL3, MOGS, ALG2, C2orf74, PUS10, KIAA0391, POP4, TSEN54, |
| | AGO4, ME3, ANKRD55, KIAA0319L, WSCD1, PNP, JAZF1, ESD, PDE4C, RGS1, LPP, |
| | SH2B3, KIAA1109, CPPED1, PPP6C, CTSA, VAT1, WDR1, TNNC2, MYH7B, PFKFB2, |
| | OXCT1, PDE3B, CPZ, LDHA, DOK7, GPX8, GPX4, PDE4A, CHRNE, NPL, TAGAP, |
| | ACBD3, TXNL4B, N4BP2, HIBCH, DGUOK, SERAC1, ABHD6, DCTPP1, CYCS, |
| | RUFY4 |

TABLE 13-20
Cluster 20 of the 67 SNP clusters.

| | |
|---|---|
| Functional | N/A |
| Annotation | |
| SNPs | rs9880289, rs6673129, rs1689510, rs2456973, rs74597067, rs965355, rs7333671, rs7787, |
| | rs4149228, rs6705628, rs2267828, rs73135369, rs73137125, rs3829578, rs2723196, |
| | rs72952888, rs56228409, rs750402, rs11866427, rs114568253, rs352834, rs1143679, |
| | rs11860650, rs13338069, rs34082782, rs34572943, rs34767000, rs35314490, rs35472514, |
| | rs36001237, rs41476751, rs9888739, rs9888879, rs7011788, rs209665, rs209677, |
| | rs12764378, rs4948496, rs9415635, rs12422045, rs3794060, rs4944946, rs11578098, |
| | rs494896, rs9415635, rs12422045, rs3794060, rs4944946, rs11578098, rs12738833, |
| | rs6662618, rs2836882, rs111476894, rs111546231, rs112430375, rs113419431, |
| | rs113779281, rs113870709, rs141868266, rs17103280, rs17103282, rs186479757, |
| | rs74392337, rs74649380, rs75092674, rs75102897, rs75695234, rs76238820, rs77825805, |
| | rs78573504, rs78609563, rs78656115, rs79294954, rs80247019, rs80313423, rs340630, |
| | rs913678, rs77150043, rs1024580, rs9651118 |
| Gene | TNIK, SKI, GDF11, SLFN11, FOXB1, KLF12, XPO4, LEFTY2, TET3, GTF2IRD1, HLF, |
| symbols | IL37, BRDT, EZH1, DNMT3B, CDH13, DNMT3A, ZBTB24, TGFB1I1, CHD7, ASIP, |
| | ARID5B, NADSYN1, PAX8, GFI1, ETS2, BAZ1A, PTPN13, ADNP, BRD7, MYOCD, |
| | MTHFR |

TABLE 13-21
Cluster 21 of the 67 SNP clusters.

| | |
|---|---|
| Functional | Regulation Of The Epithelial Mesenchymal Transition In Development |
| Annotation | Granzyme B Signaling |
| | D-myo-inositol (1, 4, 5)-trisphosphate Degradation |
| | 1D-myo-inositol Hexakisphosphate Biosynthesis II (Mammalian) |
| | D-myo-inositol (1, 3, 4)-trisphosphate Biosynthesis |
| | ciliary basal body-plasma membrane docking |
| | cilium assembly |
| | Mitotic G2-G2/M phases |
| | Genes related to primary cilium development |
| | Ciliopathies |
| SNPs | rs73161005, rs9823523, rs9855205, rs11645653, rs7186852, rs7197475, rs916287, |
| | rs74597067, rs10747210, rs10782004, rs11150608, rs11150610, rs11150612, rs11150617, |
| | rs11150619, rs1143682, rs11574630, rs11645917, rs11859274, rs12444713, rs12923297, |
| | rs2230429, rs28372932, rs36100148, rs3925074, rs3925075, rs41440449, rs4411512, |
| | rs4506917, rs4632147, rs6565201, rs72799341, rs8048448, rs9673522, rs1463485, |
| | rs3785437, rs10933559, rs13026988, rs4639966, rs503425, rs688161, rs60288561, rs2236178, |
| | rs12050151, rs2271293, rs6088765, rs58089667, rs4474733, rs2298455, rs56272032, |
| | rs281392, rs602662, rs632111, rs57545959, rs144401070, rs12598357, rs12928404, |
| | rs10101292, rs10106207, rs10108347, rs10109167, rs10109537, rs10481445, rs1057626, |
| | rs10903340, rs11250144, rs11775149, rs11783247, rs11785394, rs11989439, rs12155745, |
| | rs12677146, rs12677326, rs12677843, rs12680762, rs12682349, rs13257831, rs13260727, |
| | rs13268126, rs13272061, rs13274106, rs1382567, rs1435275, rs1478892, rs1478894, |
| | rs1478897, rs1600249, rs1600250, rs1600252, rs17153419, rs17726209, rs17782536, |
| | rs17782554, rs17783634, rs17799486, rs17807624, rs2001329, rs2043508, rs2127127, |
| | rs2244894, rs2248696, rs2248700, rs2248932, rs2251473, rs2280805, rs2409718, |
| | rs2409727, rs2409745, rs2409749, rs2409781, rs2437152, rs2572369, rs2572379, rs2572389, |
| | rs2572394, rs2572395, rs2572397, rs2572399, rs2572440, rs2572446, rs2572447, |
| | rs2572448, rs2572450, rs2618479, rs2736288, rs2736290, rs2736292, rs2736294, rs2736296, |
| | rs2736302, rs2736303, rs2736305, rs2736313, rs2736342, rs2736346, rs2736347, |
| | rs3021494, rs3021500, rs34069332, rs34389419, rs34801597, rs35009431, rs35223712, |
| | rs35499486, rs4240671, rs4333549, rs4523214, rs4549730, rs4631424, rs4840550, |
| | rs4840551, rs4840567, rs4841504, rs6601561, rs6601562, rs6601564, rs6601570, rs6984496, |
| | rs6985109, rs7000141, rs7836059, rs6663448, rs117036242, rs117519683, rs10506577, |
| | rs2380925, rs12298456, rs2241003, rs11145763, rs11145974, rs11164848, rs11578098, |
| | rs11803383, rs12738833, rs12753920, rs6662618, rs72952888, rs73039184, rs7810922, |
| | rs4482697, rs4678000, rs6438700, rs2455331, rs117371650, rs6133922, rs11154801, |
| | rs6914831, rs9402743, rs7897633, rs138759056, rs1170426, rs1170436 |
| Gene | TBC1D10A, LZTFL1, Sept1, STK38, SOCS7, ZNF668, WBP2, TRIM65, TRIM47, |
| symbols | TBC1D10B, STK25, TRAPPC4, TTC19, KIZ, CEP128, NUTF2, GDF5, CDC42EP5, TUBG2, |
| | NUMA1, FOXR1, KCNH2, FUT2, UQCR10, CATIP, VPS53, RAB34, CLN3, |
| | CTSBS100A4, ENSG00000108825, TMEM67, RAB3IP, KIF24, NEDD1, CNTRL, INPP5E, |
| | EVI5, FGFR1OP, CUX1, FBF1, IQCB1, CEP72, RAB11A, MKKS, AHI1, PRKG1, IST1, |
| | Sept2, MRPL38, CDH1 |

TABLE 13-22
Cluster 22 of the 67 SNP clusters.

| | |
|---|---|
| Functional | N/A |
| Annotation | |
| SNPs | rs17105765, rs10225904, rs3801814, rs763616, rs2256597, rs2787733, rs13026988, |
| | rs340630, rs6499034, rs8061145 |
| Gene | HOXC10, HOXC6, HOXA5, HOXA6, HOXC9, TMEM26, HOXC8, ING5, HOXA9, |
| symbols | HOXA7, AFF1, CDH11 |

TABLE 13-23
Cluster 23 of the 67 SNP clusters.

| | |
|---|---|
| Functional | N/A |
| Annotation | |
| SNPs | rs6088765, rs2297550, rs3024505, rs79074797, rs11755393, rs13205210, rs1535001, |
| | rs2499714, rs2764208, rs34840245, rs3734266, rs7764921, rs9462015, rs9462027, |
| | rs11741255, rs4639966, rs503425, rs688161, rs56228409, rs11705183, rs10893872, |
| | rs1128334, rs12575600, rs7683537 |
| Gene | GSS, IKBKE, TRAF3IP2, C6orf106, IRF1, NLRX1, BECN1, TAB1, ETS1, P4HA2, |
| symbols | CASP3 |

TABLE 13-24
Cluster 24 of the 67 SNP clusters.

| | |
|---|---|
| Functional | Acyl-CoA Hydrolysis |
| Annotation | Glutathione Redox Reactions I |
| | IL-17A Signaling in Fibroblasts |
| | Stearate Biosynthesis I (Animals) |
| | Apelin Adipocyte Signaling Pathway |
| | Fractional exhaled nitric oxide (childhood) |
| | Nitric Oxide |
| | fatty acid metabolic process |
| | Artery |
| | Cholesterol, HDL |
| SNPs | rs78712140, rs111469562, rs112345383, rs112743130, rs112876941, rs112880843, |
| | rs113115305, rs113233720, rs113370572, rs11655550, rs12232497, rs12938117, |
| | rs12945253, rs12946188, rs12946792, rs12947480, rs1453560, rs28392589, rs28535471, |
| | rs28621669, rs28648398, rs28723017, rs2941509, rs4252665, rs72832915, rs8079075, |
| | rs9303277, rs9907966, rs11998187, rs11539148, rs4625, rs62262116, rs79154635, |
| | rs11741255 |
| Gene | NFKBIZ, GSDMB, MIEN1, DLC1, LYRM9, GPX1, STARD3, ACOT7, SLC22A5 |
| symbols | |

TABLE 13-25
Cluster 25 of the 67 SNP clusters.

| | |
|---|---|
| Functional | Synaptogenesis Signaling Pathway |
| Annotation | D-myo-inositol (1,4,5,6)-Tetrakisphosphate Biosynthesis |
| | D-myo-inositol (3,4,5,6)-tetrakisphosphate Biosynthesis |
| | Dopamine-DARPP32 Feedback in cAMP Signaling |
| | 3-phosphoinositide Degradation |
| | Myocytes |
| | actomyosin structure organization |
| | Cardiomyocytes |
| | Splicing factor NOVA regulated synaptic proteins |
| | Biogenic Amine Synthesis |
| SNPs | rs1559758, rs507230, rs111469562, rs112345383, rs112743130, rs112876941, rs112880843, |
| | rs113115305, rs113370572, rs12232497, rs1453560, rs2941509, rs72832915, rs9303277, |
| | rs4802307, rs62136099, rs7186889, rs238224, rs113233720, rs11655550, rs12938117, |
| | rs12945253, rs12946188, rs12946792, rs12947480, rs28392589, rs28535471, |
| | rs28621669, rs28648398, rs28723017, rs4252665, rs8079075, rs9907966, rs7121755, |
| | rs74603582, rs74597067, rs203102, rs2455331, rs501089, rs513425, rs4971066, rs12444634, |
| | rs12918180, rs2688608, rs140207360, rs17319190, rs17319672, rs4459332, |
| | rs34006827, rs1413753, rs57681844, rs117196351, rs117303757, rs11768806, rs13237475, |
| | rs35939202, rs4728142, rs4731532, rs6592199, rs6574349, rs73066671, rs7186852, |
| | rs7197475, rs11231824, rs11171, rs79558495 |
| Gene | LDLRAD3, ZPBP2, FKRP, TCAP, CNEP1R1, MINK1, PGAP3, GSDMA, CADM1, |
| symbols | KCNAB1, CACNB1, PPP1R1B, CA10, TPPP, KIAA0195, CRYAB, GRB7, EFNA1, |
| | RBFOX1, MYOZ1, GRID2, INPPL1, KIRREL2, EPB41L2, PPP2R3A, EPB41L3, FLNC, |
| | DLG2, POMT2, ENO3, PNMT, TNNT1, MYLPF, NRXN2, ACHE |

TABLE 13-26
Cluster 26 of the 67 SNP clusters.

| | |
|---|---|
| Functional | N/A |
| Annotation | |
| SNPs | rs10952261, rs56272032 |
| Gene symbols | GIMAP8, GIMAP7, GIMAP4, GIMAP5, GIMAP6 |

TABLE 13-27
Cluster 27 of the 67 SNP clusters.

| | |
|---|---|
| Functional | N/A |
| Annotation | |
| SNPs | rs2286672, rs238224, rs79404002, rs10499197, rs17781283, rs58721818, rs670369, |
| | rs6932056, rs2241003, rs4676410, rs12992815, rs55634455, rs8012283, rs494003, |
| | rs931127, rs59494035, rs56197414, rs11738358, rs26657, rs4869313, rs913678, rs12248123, |
| | rs4917385, rs11231824, rs4579751, rs4639966, rs688161, rs4283545, rs422258, |
| | rs7566538, rs11013210, rs7916519, rs17690923, rs7411387, rs7787, rs2501800, rs112123005, |
| | rs113304138, rs4748940, rs12712067, rs73966422, rs116844988, rs1121242, rs2476601, |
| | rs11741255 |
| Gene | INCA1, OLIG3, PHF19, GPR35, LCT, NIN, OVOL1, HMP19, IFT57, CAST, STAU1, |
| symbols | NT5C2, FLRT1, KAZN, KIF1C, IFT46, ARF4, MAP1B, KIF1A, ARMC3, KIF3A, PIFO, |
| | IFT88, PADI4, PRKCQ, AFF3, KIF5A, ADAD1, PTPN22, SLC22A4 |

TABLE 13-28
Cluster 28 of the 67 SNP clusters.

| | |
|---|---|
| Functional | Axonal Guidance Signaling |
| Annotation | Semaphorin Neuronal Repulsive Signaling Pathway |
| | PI3K Signaling in B Lymphocytes |
| | Epithelial Adherens Junction Signaling |
| | Semaphorin Signaling in Neurons |
| | Rap1 signaling pathway |
| | Ras signaling pathway |
| | Leukocyte transendothelial migration |
| | Systemic lupus erythematosus |
| | Cardiovascular system disease |
| SNPs | rs72793687, rs7899961, rs2669010, rs6695567, rs10933559, rs13026988, rs11539148, |
| | rs1378942, rs281392, rs602662, rs632111, rs1496453, rs1876680, rs867709, rs3024505, |
| | rs1981601, rs111319527, rs4625, rs11065513, rs2230776, rs2688608, rs6945400, |
| | rs452999, rs771459, rs13014054, rs13385731, rs13425999, rs2271293, rs11658698, |
| | rs12801636, rs931127, rs112435140, rs2286672, rs17630235, rs114742305, rs115513376, |
| | rs142512991, rs10823062, rs425648, rs10419198, rs61919512 |
| Gene | IPMK, OSBPL8, SLC1A7, FARP2, PLXNB1, SEMA7A, RASIP1, SLC38A9, DEPTOR, |
| symbols | RASSF5, RAPGEF5, PLCE1, SEMA3F, CAMKK2, CAMK2G, GRB10, VAV2, BCAR3, |
| | RASGRP3, EDC4, RPTOR, SIPA1, MACROD2, PLD2, BRAP, SEMA5A, RHOA, |
| | SIRT1, FKBP8, RRAS, A2ML1 |

TABLE 13-29
Cluster 29 of the 67 SNP clusters.

| | |
|---|---|
| Functional | N/A |
| Annotation | |
| SNPs | rs11231814, rs4930194, rs6568431, rs7197475, rs12917712, rs10823062, rs12978179, |
| | rs12986413, rs187577845, rs72799341, rs12598357, rs12928404, rs7500321, rs494003, |
| | rs931127,rs989767,rs6565201,rs7186852,rs8048448 |
| Gene | TRMT112, C19orf52, RTN4IP1, GDPD3, PA1, YPEL3, TBX6, DNAJC12, ASPHD1, |
| symbols | IZUMO4, GRID1, FAM57B, SBK1, SEZ6L2, KAT5, PLXDC2, SETD1A |

TABLE 13-30
Cluster 30 of the 67 SNP clusters.

| | |
|---|---|
| Functional | Phospholipases |
| Annotation | Choline Biosynthesis III |
| | Antioxidant Action of Vitamin C |
| | Endothelin-1 Signaling |
| | Eicosanoid Signaling |
| | glycerophospholipid biosynthetic process |
| | phosphatidylcholine metabolic process |
| | phosphatidylserine metabolic process |
| | calcium-independent phospholipase A2 activity |
| | phospholipase activity |
| SNPs | rs60288561, rs1534154, rs3733345, rs7186852, rs7197475, rs1170426, rs1170436, |
| | rs2271293, rs34709472, rs56154925, rs4622329, rs111469562, rs112345383, rs112743130, |
| | rs112876941, rs112880843, rs113115305, rs113233720, rs113370572, rs12232497, |
| | rs12938117, rs12945253, rs12946188, rs12946792, rs12947480, rs143123127, rs1453560, |
| | rs28392589, rs28535471, rs28621669, rs28648398, rs28723017, rs2941509, rs72832915, |
| | rs8079075, rs9303277, rs9907966, rs1048257, rs4459332, rs1051390, rs1055382, |
| | rs1061502, rs1062099, rs10902173, rs11246189, rs11246213, rs11246217, rs1124816, |
| | rs1131665, rs11539530, rs12272314, rs12280580, rs12419694, rs12420647, rs12793222, |
| | rs12795572, rs12797496, rs12800061, rs12802200, rs12803048, rs12803132, rs12805435, |
| | rs2246614, rs2396545, rs28392994, rs34889521, rs35788385, rs35865896, rs36060383, |
| | rs4963127, rs4963128, rs58688157, rs6598011, rs936469, rs602662, rs632111, |
| | rs10916668, rs12408855, rs7214635, rs10197217, rs72920193, rs968567, rs12743484, |
| | rs10857712 |
| Gene | PLD6, PLAIA, DGKQ, SEPHS2, PLA2G15, TMEM86B, CHPT1, IKZF3, PLD4, ARAP1, |
| symbols | RNH1, PTDSS2, PLA2G4C, PLA2G5, ALOX15, B3GNT2, FADS2, PAFAH2, CYP2E1 |

TABLE 13-31
Cluster 31 of the 67 SNP clusters.

| | |
|---|---|
| Functional | N/A |
| Annotation | |
| SNPs | rs1378942, rs2289583, rs7172677, rs1072626, rs600751, rs3731570, rs1360328, |
| | rs1133950, rs2271293, rs10922144, rs7731626, rs1170426, rs10857712, rs56262793, |
| | rs72787037, rs11085735, rs118115488, rs12720356, rs147622113, rs2304257, rs34536443, |
| | rs74908652, rs4783754, rs62131887, rs6574349, rs57545959 |
| Gene | PPCDC, TRMT10A, RPUSD4, COQ10B, TRMT11, UTP23, FTSJ2, DUS2, CFHR3, |
| symbols | CFHR4, DHX29, CFHR1, EDC3, DDX28, MTG1, DHODH, KRI1, KCTD19, PPAN, |
| | SAMD15, AAMP |

TABLE 13-32
Cluster 32 of the 67 SNP clusters.

| | |
|---|---|
| Functional | Putrescine Biosynthesis III |
| Annotation | Hypertrophy of heart |
| | Hematopoiesis of liver |
| | Increased localization of ALT |
| | Cardiac Hypertrophy |
| | Mitochondrial iron-sulfur cluster biogenesis Homo sapiens |
| | positive regulation of histone ubiquitination |
| | ChREBP activates metabolic gene expression |
| | Polyamine metabolism |
| | ERBB1 internalization pathway |
| SNPs | rs56228409, rs1332099, rs74597067, rs209665, rs209677, rs6565201, rs7186852, rs7197475, |
| | rs72799341, rs8048448, rs223881, rs223883, rs4690055, rs6705628, rs72793687, |
| | rs7899961, rs2407782 |
| Gene | MLX, SLC25A28, NARF, CDK5RAP1, XRCC6BP1, RNF40, MNT, CIAPIN1, FDX1L, |
| symbols | ZFYVE28, BOLA3, MXD4, AZIN2, CISD1, KIAA2018, TWF1 |

TABLE 13-33
Cluster 33 of the 67 SNP clusters.

| | |
|---|---|
| Functional | nNOS Signaling in Skeletal Muscle Cells |
| Annotation | Insulin Secretion Signaling Pathway |
| | Netrin Signaling |
| | Synaptogenesis Signaling Pathway |
| | Maturity Onset Diabetes of Young (MODY) Signaling |
| | synaptic vesicle exocytosis |
| | SNARE interactions in vesicular transport |
| | regulation of ventricular cardiac muscle cell action potential |
| | Arrhythmogenic Right Ventricular Cardiomyopathy |
| | Cardiac muscle contraction |
| SNPs | rs1378942, rs2289583, rs7172677, rs10747210, rs10782004, rs11150612, rs11150617, |
| | rs1143682, rs11574630, rs11641202, rs11645917, rs11859274, rs12923297, rs28372932, |
| | rs36100148, rs3925074, rs3925075, rs41440449, rs4411512, rs4506917, rs4632147, |
| | rs6565201, rs72799341, rs8048448, rs9673522, rs34099611, rs79821347, rs73066671, |
| | rs10773782, rs13332545, rs36001237, rs7186852, rs7197475, rs9937837, rs11539148, |
| | rs4625, rs62262116, rs26657, rs4869313, rs7533588, rs35370245, rs4474733, |
| | rs56228409, rs74597067, rs145992193, rs2283293, rs1463485, rs3785437 |
| Gene | SCAMP5, STX4, SCRIB, CACNA2D2, CPLX3, SYT5, RIMBP2, STX1, SCAMP2, |
| symbols | BSN, LNPEP, SNAP47, DLG1, AOC3, CACNA1C, UNC13D |

TABLE 13-34
Cluster 34 of the 67 SNP clusters.

| | |
|---|---|
| Functional | Bupropion Degradation |
| Annotation | Phagosome Formation |
| | Acetone Degradation I (to Methylglyoxal) |
| | Estrogen Biosynthesis |
| | Oxidative Ethanol Degradation III |
| | Lysosome Vesicle Biogenesis Homo sapiens |
| | trans-Golgi Network Vesicle Budding Homo sapiens |
| | Clathrin derived vesicle budding Homo sapiens |
| | post-Golgi vesicle-mediated transport |
| | regulation of insulin receptor signaling pathway |
| SNPs | rs1230666, rs1237290, rs2476601, rs6679677, rs2804278, rs11013210, rs7011788, |
| | rs11085727, rs147622113, rs2304256, rs956237, rs4958893, rs4958894 |
| Gene | PHTF1, KANK1, PIP4K2A, MAGI3, RSBN1, AP4B1, CLVS1, AP1M2, CYP2U1, |
| symbols | ANXA6 |

TABLE 13-35
Cluster 35 of the 67 SNP clusters.

| | |
|---|---|
| Functional | N/A |
| Annotation | |
| SNPs | rs7679, rs1193329, rs1860598, rs6913643, rs4544377, rs1028488, rs910425, rs12232497, |
| | rs72832915, rs9303277, rs6049839, rs74546614, rs1369481, rs1053093, rs10911390, |
| | rs41263646, rs10156169, rs10229001, rs10236569, rs10239340, rs10254963, |
| | rs10279821, rs10488631, rs1072767, rs1077481, rs1077482, rs10954213, rs10954214, |
| | rs113253472, rs11763959, rs11767954, rs11768806, rs11972310, rs11972719, rs12155080, |
| | rs12531054, rs12531711, rs12535158, rs12536266, rs12537264, rs12537284, |
| | rs12539476, rs12539741, rs12706861, rs12706862, rs13222807, rs13225818, rs13227095, |
| | rs13236009, rs13237475, rs13239597, rs13240595, rs13244090, rs17339773, rs17339836, |
| | rs17340351, rs17340646, rs17424602, rs17425212, rs2070197, rs2242028, rs2272347, |
| | rs2280714, rs34725944, rs35000415, rs35188261, rs35234849, rs35939202, |
| | rs3757385, rs3757387, rs3778750, rs3778753, rs3807306, rs3807307, rs3847099, |
| | rs4728142, rs4728144, rs4728152, rs4731530, rs4731532, rs4731533, rs4731536, |
| | rs4731540, rs4731541, rs4731548, rs4731549, rs59110799, rs6467228, rs6467229, rs7808907, |
| | rs912784, rs464694, rs12248123, rs4917385, rs6697139, rs139810877, rs115073033, |
| | rs6589566, rs662799, rs12470957, rs11655550, rs1194, rs6938946, rs10101292, |
| | rs10106207, rs10108347, rs10109025, rs10109167, rs10109537, rs10481445, rs1057626, |
| | rs10903340, rs11250144, rs11775149, rs11783247, rs11785394, rs11989439, |
| | rs12155745, rs12677146, rs12677326, rs12677843, rs12680762, rs12682349, rs13257831, |
| | rs13260727, rs13268126, rs13272061, rs13274106, rs13277113, rs1382567, rs1435275, |
| | rs1478892, rs1478894, rs1478897, rs1600249, rs1600250, rs1600252, rs17153419, |
| | rs17726209, rs17782536, rs17782554, rs17783634, rs17799486, rs17807624, rs2001329, |
| | rs2043508, rs2061831, rs2127127, rs2244894, rs2248696, rs2248700, rs2248932, |
| | rs2251473, rs2254546, rs2280805, rs2409718, rs2409727, rs2409745, rs2409749, |
| | rs2409781, rs2437152, rs2572369, rs2572379, rs2572389, rs2572394, rs2572395, rs2572397, |
| | rs2572399, rs2572440, rs2572446, rs2572447, rs2572448, rs2572450, rs2618458, |
| | rs2618473, rs2618476, rs2618479, rs2736288, rs2736290, rs2736292, rs2736294, |
| | rs2736296, rs2736302, rs2736303, rs2736305, rs2736313, rs2736332, rs2736340, rs2736342, |
| | rs2736345, rs2736346, rs2736347, rs2736350, rs3021494, rs3021500, rs34069332, |
| | rs34389419, rs34801597, rs35009431, rs35223712, rs35499486, rs4240671, rs4333549, |
| | rs4523214, rs4549730, rs4631424, rs4840550, rs4840551, rs4840567, rs4840568, |
| | rs4841504, rs6601561, rs6601562, rs6601564, rs6601570, rs6984496, rs6985109, |
| | rs7000141, rs7820492, rs7836059, rs968567, rs6780105, rs913678 |
| Gene | PCIF1, SH2D4A, FAM184B, PDCD2, NR4A2, FAM120B, MED24, MED28, TMC2, |
| symbols | KCNIP4, NPAS2, RGL1, TNPO3, ZC3H13, MED15, WBP1L, USF1, SPG11, |
| | ARHGAP26, TAGLN, MAT2A, MED1, PPARD, FDFT1, FADS1, RARB, CEBPB |

TABLE 13-36
Cluster 36 of the 67 SNP clusters.

| | |
|---|---|
| Functional | Phenylethylamine Degradation I |
| Annotation | RAC Signaling |
| | Sphingomyelin Metabolism |
| | Signaling by Rho Family GTPases |
| | Complement System |
| | Type II diabetes mellitus |
| | sensory perception |
| | Cell projection |
| | USH2 complex |
| | ligand-gated cation channel activity |
| SNPs | rs3780205, rs944333, rs2489159, rs2675954, rs7759743, rs16867384, rs918959, rs10101292, |
| | rs10106207, rs10108347, rs10109025, rs10109167, rs10481445, rs1057626, rs10903340, |
| | rs11783247, rs11989439, rs12677146, rs13272061, rs1382567, rs1600250, |
| | rs1600252, rs17726209, rs17782536, rs17782554, rs17783634, rs17799486, rs17807624, |
| | rs2001329, rs2127127, rs2251473, rs2409718, rs2409727, rs2409745, rs2409749, rs2572397, |
| | rs2736313, rs3021494, rs3021500, rs34069332, rs35223712, rs35499486, rs4333549, |
| | rs4523214, rs4840550, rs4840551, rs4840567, rs4841504, rs6601561, rs6601562, rs6601564, |
| | rs6601570, rs6984496, rs6985109, rs7000141, rs7831557, rs7836059, rs1670807, |
| | rs2891551, rs11813373, rs533852, rs13418915, rs62130302, rs6742110, rs7575676, rs7590355, |
| | rs2271293, rs73152525, rs10922144, rs352834, rs436872, rs2786111, rs6689858, |
| | rs4474733, rs74597067, rs17124948, rs8011382, rs7712962, rs11804305 |
| Gene | NEK6, DENND1A, KCNJ13, EYS, CERKL, RP1L1, PCDH15, CDH23, IMPG2, C2orf71, |
| symbols | PARD6A, DGKI, CFH, SMPD2, CRB1, AOC2, TTC8, CYFIP2, CHRNB2 |

TABLE 13-37
Cluster 37 of the 67 SNP clusters.

| | |
|---|---|
| Functional | |
| Annotation | |
| SNPs | rs13252899, rs34099611 |
| Gene symbols | SLC39A4, FBXL6, TMEM249, SLC52A2, ZNF707, ADCK5 |

TABLE 13-38
Cluster 38 of the 67 SNP clusters.

| | |
|---|---|
| Functional | N/A |
| Annotation | |
| SNPs | rs645078, rs6695567, rs6589566, rs662799, rs4639966, rs17301013, rs9782955, |
| | rs907526, rs17413405, rs17483887, rs6705628, rs7625006 |
| Gene | FKBP2, C1orf123, ZPR1, HYOU1, TROVE2, SLC9C2, ERO1LB, GRPEL1, HSCB, |
| symbol | ANKRD45, TTC28, AUP1, MANF |

TABLE 13-39
Cluster 39 of the 67 SNP clusters.

| | |
|---|---|
| Functional | N/A |
| Annotation | |
| SNPs | rs17630235, rs12148050, rs2015407, rs1133906, rs4474733, rs74597067, rs6920432, |
| | rs114273055, rs956237, rs72743477, rs2241003, rs147622113, rs2304087, rs726848, |
| | rs56228409 |
| Gene | TRAFD1, JADE2, TRAF3, SAMD9L, IFI35, FKBP5, NBR1, IQSEC1, LEF1, SMAD3, |
| symbol | TRAF1, SMARCA4, SMARCE1, RND2 |

TABLE 13-40
Cluster 40 of the 67 SNP clusters.

| | |
|---|---|
| Functional | tRNA Splicing |
| Annotation | Role of OCT4 in Mammalian Embryonic Stem Cell Pluripotency |
| | Relaxin Signaling |
| | eNOS Signaling |
| | Cardiac β-adrenergic Signaling |
| | response to endoplasmic reticulum stress |
| | disulfide oxidoreductase activity |
| | AMP binding |
| | Nitric oxide metabolism |
| | Vascular Endothelial Cell Activation by NO |
| SNPs | rs2303745, rs10419198, rs13000817, rs10142203, rs500600, rs58089667 |
| Gene | NR2F6, PRRG2, RCN3, PRR12, FAM136A, TMX1, MPPED2, ARL14EP, NOSIP, |
| symbols | LENG8 |

TABLE 13-41
Cluster 41 of the 67 SNP clusters.

| | |
|---|---|
| Functional | N/A |
| Annotation | |
| SNPs | rs6073958, rs931127, rs4544377, rs10988542, rs12620999, rs12928404, rs7500321, |
| | rs6705628, rs4888360, rs12142199, rs750402, rs11681903, rs4474733, rs74597067, |
| | rs7679, rs4917385, rs1048257, rs7172677, rs73286503, rs6492983, rs12598357, rs11024600, |
| | rs4802307, rs62136099, rs11756587, rs117371650, rs4639966, rs503425, rs688161, |
| | rs3783759, rs12917712, rs7197475, rs72799341 |
| Gene | SPATA25, EIF1AD, GPD2, TOR1B, COPS8, EIF3CL, STAMBP, PSMD7, SSU72, |
| symbols | COMMD7, CCDC93, DHX8, NEURL2, CALHM2, NUDT14, COMMD4, C5orf24, INO80, |
| | LAT, GTF2H1, TRIM25, HIF3A, GTF2H5, DENND4A, DDX6, MNAT1, INO80E |

TABLE 13-42
Cluster 42 of the 67 SNP clusters.

| | |
|---|---|
| Functional | N/A |
| Annotation | |
| SNPs | rs3769433, rs62262116, rs79821347, rs3733345, rs17301013, rs4573125, rs7946044, |
| | rs501089, rs513425, rs11539148, rs351758, rs4625, rs645078, rs7042370, rs74597067, |
| | rs144484752, rs4648892, rs7280984, rs7172677, rs1049564, rs12588573, rs34709472, |
| | rs56154925, rs73161005, rs3731570, rs6738825, rs6705628, rs1429411, rs4850410, |
| | rs4690055, rs2019097, rs11152964, rs12526490, rs870707 |
| Gene | SPATS2L, IFRD2, SLC26A1, DARS2, FARS2, NARS2, FDXACB1, DALRD3, ELP3, |
| symbol | WDR6, CCDC88B, LURAP1L, NUFIP2, SLC25A21, ASAP3, HLCS, MAN2C1, OSGEP, |
| | PPP6R1, TYW5, ASCC2, RFTN2, SLC4A5, ANKRD44, SH3BP2, FAM126B, |
| | QRSL1, CLDN10 |

TABLE 13-43
Cluster 43 of the 67 SNP clusters.

| | |
|---|---|
| Functional | nNOS Signaling in Skeletal Muscle Cells |
| Annotation | Synaptogenesis Signaling Pathway |
| | Sperm Motility |
| | Notch Signaling |
| | Inhibition of Matrix Metalloproteases |
| | amyloid-beta metabolic process |
| | neutrophil degranulation |
| | neutrophil activation involved in immune response |
| | neutrophil mediated immunity |
| | EphrinB-EPHB pathway Homo sapiens |
| SNPs | rs17036143, rs11085727, rs118115488, rs12720356, rs2304256, rs2304257, rs2569702, |
| | rs2569703, rs280497, rs34725611, rs7258015, rs10419198, rs116844988, rs4625, |
| | rs73881943, rs116660017, rs79558495, rs2982846, rs34743561, rs230261, rs34006827, |
| | rs117826817, rs2220168, rs62149377, rs1830847, rs4482697, rs11539148, rs62262116, |
| | rs164178, rs3024505, rs1565123, rs11705183, rs16826235, rs1061511, rs10282742 |
| Gene | TIMP4, ICAM5, FCGRT, TSPAN31, DAG1, SYN2, SYN3, FCRLA, EPHB4, RAB3B, |
| symbols | APLP1, TPD52, ITPR2, PAPOLG, FCRL4, HCLS1, APEH, NOS1AP, PIGR, LRMP, |
| | SYNGR1, ZNF804A, NCSTN, ABCA13 |

TABLE 13-44
Cluster 44 of the 67 SNP clusters.

| | |
|---|---|
| Functional | Androgen Biosynthesis |
| Annotation | Estrogen Biosynthesis |
| | Bile Acid Biosynthesis, Neutral Pathway |
| | Estrogen-Dependent Breast Cancer Signaling |
| | Retinoate Biosynthesis I |
| | steroid biosynthetic process |
| | steroid dehydrogenase activity, acting on the CH—OH group of donors, NAD or |
| | NADP as acceptor |
| | Steroid hormone, vitamin A, and vitamin D metabolism |
| | Lipid and lipoprotein metabolism |
| | Bile acid and bile salt biosynthesis via 27-hydroxycholesterol |
| SNPs | rs2061776, rs10419198, rs10747210, rs10782004, rs11150608, rs11150610, rs11150612, |
| | rs11150617, rs11150619, rs1143679, rs1143682, rs11574630, rs11641202, rs11645653, |
| | rs11645917, rs11859274, rs12444713, rs12923297, rs13332545, rs2230429, |
| | rs2359661, rs28372932, rs34572943, rs36100148, rs3925074, rs3925075, rs41440449, |
| | rs41476751, rs4411512, rs4506917, rs6565201, rs7186852, rs7197475, rs72799341, |
| | rs8048448, rs9673522, rs56228409, rs149002524, rs11951576, rs1378942, rs145698618, |
| | rs4468247 |
| Gene | FBN3, HSD17B14, HSD3B7, HSD17B1, AKR1C4, SRD5A1, CYP11A1, AKR1C3 |
| symbols | |

TABLE 13-45
Cluster 45 of the 67 SNP clusters.

| | |
|---|---|
| Functional | Role of Hypercytokinemia/hyperchemokinemia in the Pathogenesis of Influenza |
| Annotation | Role of RIG1-like Receptors in Antiviral Innate Immunity |
| | Role of Cytokines in Mediating Communication between Immune Cells |
| | CNTF Signaling |
| | Activation of IRF by Cytosolic Pattern Recognition Receptors |
| | cytokine-mediated signaling pathway |
| | Jak-STAT signaling pathway |
| | Cytokine-cytokine receptor interaction |
| | Complement |
| | Reactive Oxygen Species Pathway |
| SNPs | rs11671087, rs2082418, rs7359953, rs1509461, rs979233, rs3024493, rs3024505, |
| | rs3122605, rs75010564, rs12971295, rs13344313, rs10419198, rs1051390, rs1055382, |
| | rs1061502, rs1062099, rs10902173, rs11246189, rs11246213, rs11246217, rs11246262, |
| | rs11246268, rs1124816, rs1131665, rs11539530, rs12272314, rs12280580, rs12419694, |
| | rs12793222, rs12795572, rs12797496, rs12800061, rs12802200, rs12803048, rs12803132, |
| | rs12805435, rs2246614, rs2396545, rs28392994, rs34889521, rs35788385, rs35865896, |
| | rs36060383, rs4963127, rs4963128, rs4963166, rs58688157, rs6598011, rs936469, |
| | rs112329115, rs112455696, rs112610233, rs113095313, rs113743276, rs114791303, |
| | rs16993569, rs16993576, rs16993577, rs16993578, rs16993582, rs16993583, rs16993585, |
| | rs2242781, rs2242782, rs2242785, rs2834998, rs2835106, rs2835120, rs58716870, |
| | rs59031468, rs59441655, rs59507468, rs61127290, rs7277126, rs73365739, rs73365744, |
| | rs73365745, rs73365749, rs73365750, rs73365755, rs73365764, rs73365765, rs73365766, |
| | rs73365769, rs73365771, rs73365774, rs73365775, rs73365797, rs75784138, rs75826569, |
| | rs76521500, rs77481934, rs930297, rs959260, rs9894206, rs10732993, rs10737299, |
| | rs10753074, rs10798176, rs10798269, rs10912610, rs11577640, rs12752692, |
| | rs1342033, rs1342034, rs1539255, rs1547811, rs1935325, rs1935327, rs1985177, rs2027500, |
| | rs2039982, rs2039983, rs2185702, rs2422341, rs4916342, rs6425218, rs6425223 |
| Gene | IFNL2, GHR, IL19, LIFR, CRLF1, IL24, IL20, IRF3, IRF7, RUNX1, GRB2, PRDX6 |
| symbols | |

TABLE 13-46
Cluster 46 of the 67 SNP clusters.

| | |
|---|---|
| Functional | N/A |
| Annotation | |
| SNPs | rs11539148, rs4625, rs11185602, rs11185603, rs17133826, rs4917014, rs501089, |
| | rs513425, rs921916, rs7939069 |
| Gene | KLHDC8B, FIGNL1, LRRC74A, ZPBP, C11orf1, C7orf72, AGBL2 |
| symbol | |

TABLE 13-47
Cluster 47 of the 67 SNP clusters.

| | |
|---|---|
| Functional | N/A |
| Annotation | |
| SNPs | rs74597067, rs1364989, rs12096737, rs6920432, rs12971295, rs13344313, rs2244272, |
| | rs1061511, rs183211, rs962885, rs45444697, rs928596, rs4644492, rs4388254, |
| | rs7726414, rs4639966, rs503425, rs147558730, rs117372389, rs77150043, rs73066671, |
| | rs17885785 |
| Gene | SOST, GSX2, MIXL1, TEAD3, SSBP4, ONECUT1, VANGL2, WNT3, CCDC155, |
| symbols | ISL1, HCN3, WNT2B, TCF7, BCL9L, ROR2, KANSL1, NKD1, TNNI3, TH |

TABLE 13-48
Cluster 48 of the 67 SNP clusters.

| | |
|---|---|
| Functional | N/A |
| Annotation | |
| SNPs | rs12801636, rs34099611, rs494003, rs931127, rs533852 |
| Gene symbols | KCNK7, FAM89B, FAM83H, MAP3K11, LTBP3, ABI3BP |

TABLE 13-49
Cluster 49 of the 67 SNP clusters.

| | |
|---|---|
| Functional | N/A |
| Annotation | |
| SNPs | rs2891663, rs4660116, rs6673121, rs9782955, rs2425223, rs4850410, rs17759986, |
| | rs1496453, rs1860598, rs56154925, rs73066671, rs34550882, rs6565201, rs7186852, |
| | rs7197475, rs8048448, rs962885, rs4283545, rs11681718, rs5760898, rs230261, rs34006827, |
| | rs13333835, rs2958647, rs11024600, rs425648, rs183211, rs6492983, rs12978179, |
| | rs12986413, rs2732159, rs1804182, rs11539148, rs62262116, rs79821347, rs494003, |
| | rs931127, rs930297, rs959260, rs9894206, rs57956751, rs13418915, rs62130302, rs6742110, |
| | rs7575676, rs7590355, rs12666575 |
| Gene | LYST, DLGAP4, DNAH7, DNAH6, CCNO, MCIDAS, LAP3, DNAAF3, BCL7C, |
| symbols | PLEKHM1, FAM101B, DNAH12, ILIRL2, CRYBB3, NFKBID, C16orf95, ENTHD1, |
| | HPS5, MAST3, ARL17B, ARL17A, VPS18, AP3D1, AP3B2, AP3M2, MON1A, AP5B1, |
| | GGA3, TUBB6, CLIP4, APPL1, SNX8 |

TABLE 13-50
Cluster 50 of the 67 SNP clusters.

| | |
|---|---|
| Functional | Fc receptor complex |
| Annotation | Lymphoid cells in Heart |
| | Coronary atherosclerosis (increased number of diseased vessels) (traffic exposure |
| | interaction) |
| | phospholipid scramblase activity |
| | Cholesterol, HDL |
| SNPs | rs1830847, rs9427315, rs4622329, rs7197475, rs1051390, rs1055382, rs1061502, |
| | rs1062099, rs10902173, rs11246189, rs11246213, rs11246217, rs11246262, rs11246268, |
| | rs1124816, rs1131665, rs11539530, rs12272314, rs12280580, rs12419694, rs12420647, |
| | rs12793222, rs12795572, rs12797496, rs12800061, rs12802200, rs12803048, rs12803132, |
| | rs12805435, rs2246614, rs2396545, rs28392994, rs34889521, rs35788385, rs35865896, |
| | rs36060383, rs4963127, rs4963128, rs4963166, rs58688157, rs6598011, rs936469, |
| | rs352834, rs436872, rs73152987, rs3024505 |
| Gene | FCRL5, FCRL1, GNPTAB, CMIP, DEAF1, CD164, PLSCR2, RASSF7, FCAMR, |
| symbols | ANO9, TMEM80, |

TABLE 13-51
Cluster 51 of the 67 SNP clusters.

| | |
|---|---|
| Functional | Response to selective serotonin reuptake inhibitors and depression |
| Annotation | Metabolite levels (Pyroglutamine) |
| | plasma membrane invagination |
| | phagocytosis, engulfment |
| | exocytic and synaptic vesicle membranes |
| SNPs | rs74597067, rs10156091, rs6945305, rs11685321, rs16837131, rs2019097 |
| Gene | TSSC1, RUNDC1, ICA1, GULP1, CCDC132, ALS2CR12 |
| symbols | |

TABLE 13-52
Cluster 52 of the 67 SNP clusters.

| | |
|---|---|
| Functional | N/A |
| Annotation | |
| SNPs | rs238224, rs7625006, rs1441904, rs1194, rs6920432, rs6938946, rs820077, rs13205210, |
| | rs13207858, rs1535001, rs2764208, rs34840245, rs3734266, rs7764921, rs205284, |
| | rs76918338, rs10498070 |
| Gene | SPAG7, TEX264, CCDC158, ZNF76, TCP11, SCUBE3, ANKS1B, CNPPD1 |
| symbols | |

TABLE 13-53
Cluster 53 of the 67 SNP clusters.

| | |
|---|---|
| Functional | N/A |
| Annotation | |
| SNPs | rs894956, rs497273, rs11685321, rs6886392, rs11705183, rs10496726, rs11887156, |
| | rs2460382, rs4293757, rs78921499, rs2688608 |
| Gene | MGAT5B, SPPL3, DIRC1, ST8SIA4, MGAT3, MGAT5, PCDH7, FUT11 |
| symbols | |

TABLE 13-54
Cluster 54 of the 67 SNP clusters.

| | |
|---|---|
| Functional | N/A |
| Annotation | |
| SNPs | rs5760894, rs5760898, rs1830847, rs1539414, rs142105249, rs1997392, rs11741255 |
| Gene | KIAA1671, ETV3L, LPHN3, DENND1B, M1AP, ZNF385B, LRRIQ4, LRRC34, |
| symbols | MYNN, ACSL6 |

TABLE 13-55
Cluster 55 of the 67 SNP clusters.

| | |
|---|---|
| Functional | N/A |
| Annotation | |
| SNPs | rs10798176, rs6032198, rs6131014, rs209665, rs209677, rs7522008, rs223881, rs223883, |
| | rs1463485, rs3785437, rs34099611, rs10521318, rs11117431, rs11641016, rs11649485, |
| | rs17445836, rs2055802, rs7692950, rs60288561, rs1689510, rs2456973, rs2071929, |
| | rs12588573, rs930297, rs959260, rs9894206, rs7325747, rs2669010, rs1429411, |
| | rs7197475, rs4459332, rs6088765, rs1997392, rs771459 |
| Gene | MYOC, PIGTPXMP4, PIGK, DOK4, GALK1, ZC3H3, MTHFSD, NFXL1, PIGC, FLCN, |
| symbols | SUOX, SUCO, FOXF1, GSE1, EMC8, C16orf74, RNASE10, LLGL2, UBAC2, |
| | ZDHHC17PGAP1, ARL6IP1, STARD10, NCOA6, ACTRT3, PIGU, TMED5 |

TABLE 13-56
Cluster 56 of the 67 SNP clusters.

| | |
|---|---|
| Functional | Sumoylation Pathway |
| Annotation | Role of OCT4 in Mammalian Embryonic Stem Cell Pluripotency |
| | S-methyl-5-thio-α-D-ribose 1-phosphate Degradation |
| | Acute Myeloid Leukemia Signaling |
| | Non-Small Cell Lung Cancer Signaling |
| | peptidyl-lysine modification |
| | regulation of transcription by RNA polymerase II |
| | regulation of transcription, DNA-templated |
| | Basic mechanisms of SUMOylation |
| | SUMOylation as a mechanism to modulate CtBP-dependent gene responses |
| SNPs | rs533852, rs10954650, rs59953583, rs6916001, rs9482510, rs72928038, rs12664535, |
| | rs12916690, rs1869486, rs17603856, rs909788, rs138801602, rs645078, rs7280984, |
| | rs12065623, rs2238573, rs2039086, rs9556721, rs9556722, rs16837131, rs10466455, |
| | rs10742326, rs1355223, rs2553772, rs2732549, rs2785197, rs387619, rs507230, rs2303108, |
| | rs7248181, rs1250563, rs4242897, rs726848, rs7172677, rs117818071, rs12597935 |
| Gene | SENP7, HIPK2, NKAIN2, CASP8AP2, ZNF451, RORA, ATXN1, SENP5, PRDX5, SIM2, |
| symbols | RUNX3, ARID3A, RAP2A, SUMO1, APIP, SAE1, ZMIZ1, PHC1, RARA, SIN3A, |
| | ZFHX3 |

TABLE 13-57
Cluster 57 of the 67 SNP clusters.

| | |
|---|---|
| Functional | N/A |
| Annotation | |
| SNPs | rs2380925, rs13252899, rs6492983, rs10747210, rs10782004, rs11574630, rs4632147, |
| | rs6565201, rs7186852, rs7197475, rs72799341, rs8048448, rs12598357, rs12928404, |
| | rs7500321, rs2407782, rs11793585, rs143810807, rs1061511, rs11539148, rs62262116, |
| | rs16837131, rs2019097, rs73161005, rs17630235, rs11741255, rs1131265, rs1132200, |
| | rs6804441, rs928596, rs10991892, rs6088765, rs6574349, rs3731570, rs600751, |
| | rs185554382 |
| Gene | KIAA1161, VPS28, ZFYVE19, UBAP1, ZNF646, SULT1A1, PUS7L, MVB12B, |
| symbols | PEA15, CCDC36, NIF3L1, RNF215, NAA25, C5orf56, TIMMDC1, GON4L, AUH, |
| | NFS1, CHURC1, MARS2, FOXRED1, NDUFAF1, ACAD9 |

TABLE 13-58
Cluster 58 of the 67 SNP clusters.

| | |
|---|---|
| Functional | N/A |
| Annotation | |
| SNPs | rs118128424, rs2271293, rs11647067, rs138759056, rs139831076, rs6477694, rs10202434, |
| | rs10419198, rs4810485, rs6073958, rs6131014, rs7679, rs11717455, rs140870671, |
| | rs12142199 |
| Gene | WNK2, SLC12A4, SCNN1G, PKD1L3, EPB41L4B, COL6A3, TRPM4, SLC12A5, |
| symbols | SCN10A, SCNN1D, ACAP3 |

TABLE 13-59
Cluster 59 of the 67 SNP clusters.

| | |
|---|---|
| Functional | N/A |
| Annotation | |
| SNPs | rs645078, rs1292043, rs771459, rs726848, rs9303277, rs907526, rs118152395, |
| | rs12598357, rs12928404, rs7500321, rs13061150, rs6477694 |
| Gene | RPS6KA4, RNFT1, DR1, MSL1, TADA2B, CCDC101, ZBTB21, NUPR1, GRAMD1C, |
| symbols | NAA50, ATP6V1A, IKBKAP |

TABLE 13-60
Cluster 60 of the 67 SNP clusters.

| | |
|---|---|
| Functional | GABA Receptor Signaling |
| Annotation | Gustation Pathway |
| | Neuroinflammation Signaling Pathway |
| | WNT/Ca + pathway |
| | Phospholipases |
| | GABAergic synapse |
| | GABA (A) receptor activation |
| | Ligand-gated ion channel transport |
| | Cardiac Protection Against ROS Homo sapiens |
| | Vascular tissue |
| SNPs | rs73245892, rs1429411, rs3731570, |
| | rs6738825, rs7439087 |
| Gene symbols | GABRA4, PLCL1, GABRB1 |

TABLE 13-61
Cluster 61 of the 67 SNP clusters

| | |
|---|---|
| Functional | Dermatan Sulfate Degradation (Metazoa) |
| Annotation | Neuroprotective Role of THOP1 in Alzheimer's Disease |
| | NRF2-mediated Oxidative Stress Response |
| | LPS/IL-1 Mediated Inhibition of RXR Function |
| | urate transmembrane transporter activity |
| | urate transport |
| | urate metabolic process |
| | Abnormality of purine metabolism |
| | sulfur compound catabolic process |
| SNPs | rs6073958, rs7679, rs6131014, rs10101292, rs10481445, rs11775149, rs11785394, |
| | rs11989439, rs12155745, rs13272061, rs1382567, rs1600250, rs1600252, rs17799486, |
| | rs2251473, rs2409718, rs2409745, rs2409749, rs2572379, rs2572397, rs2736313, rs2736342, |
| | rs3021494, rs34069332, rs35499486, rs4333549, rs4523214, rs4840551, rs4840567, |
| | rs4841504, rs6601561, rs6601562, rs6601564, rs6601570, rs7831557, rs3733345, |
| | rs11658698, rs1473176, rs138584427, rs6557142, rs7712962, rs11089629, rs11089637, |
| | rs12158299, rs12484550, rs131654, rs131665, rs140489, rs140492, rs140498, rs140499, |
| | rs181360, rs181362, rs181363, rs181366, rs2070512, rs2256609, rs2266959, rs2266961, |
| | rs2266963, rs2283790, rs2298428, rs2298429, rs3747093, rs463426, rs464694, rs4821108, |
| | rs4821112, rs4821116, rs4821124, rs5754217, rs5754467, rs5998619, rs73166632, |
| | rs7444, rs10995092, rs573775, rs6538678, rs9952980, rs11741255, rs11076230, rs1974876, |
| | rs2288012, rs12588573, rs11580078, rs10822050, rs224031, rs77029323, rs11231824, |
| | rs6449173, rs7442295, rs1189433, rs1751036 |
| Gene | SNX21, WFDC3, PRSS55, IDUA, NPTX1, CUTC, CCDC170, RMND1, C5orf52, |
| symbols | CCDC116, RTKN2, C6orf203, CCDC38, SLC14A2, C6orf211, SHROOM1, CCDC113, |
| | ANG, C1orf141, ADO, SLC22A12, SLC2A9, ABCC4 |

TABLE 13-62
Cluster 62 of the 67 SNP clusters

| | |
|---|---|
| Functional | N/A |
| Annotation | |
| SNPs | rs10466455, rs10742326, rs1559758, rs387619, rs507230, rs1292043, rs7252175, |
| | rs183211, rs962885, rs73156242 |
| Gene | PAMR1, SLC1A2, TUBD1, EML2, MAPT, PVALB |
| symbols | |

TABLE 13-63
Cluster 63 of the 67 SNP clusters

| | |
|---|---|
| Functional | N/A |
| Annotation | |
| SNPs | rs600751, rs1131265, rs1132200, rs6804441, rs10521318, rs11117431, rs11641016, |
| | rs11649485, rs17445836, rs6492983, rs112018349, rs7915387, rs6705304, rs6756736, |
| | rs835574, rs10822050, rs1028488, rs910425 |
| Gene | ST3GAL4, POGLUT1, FOXC2, DLL4, C8orf4, MYT1L, UNC5B, THADA, NOTCH2, |
| symbols | ZNF365, DLL1 |

TABLE 13-64
Cluster 64 of the 67 SNP clusters

| | |
|---|---|
| Functional | p53 Signaling |
| Annotation | Calcium Signaling |
| | Cul4-RING E3 ubiquitin ligase complex |
| | calcium ion transmembrane transport |
| | mannosyl-oligosaccharide mannosidase activity |
| | Ion channel function in vascular endothelium |
| | Nitric Oxide Effects on beta-Cell |
| SNPs | rs1051390, rs1055382, rs1061502, rs1062099, rs10902173, rs11246189, rs11246213, |
| | rs1124816, rs1131665, rs11539530, rs12272314, rs12280580, rs12419694, rs12420647, |
| | rs12793222, rs12795572, rs12797496, rs12800061, rs12802200, rs12803048, rs12803132, |
| | rs12805435, rs2246614, rs2396545, rs34889521, rs35788385, rs35865896, rs36060383, |
| | rs4963127, rs4963128, rs58688157, rs936469, rs6131014, rs10499197, rs17781283, |
| | rs58721818, rs670369, rs6932056, rs6088765, rs73170472, rs2303745 |
| Gene | LMNTD2, WFDC13, PERP, WDR42A, TRPC4AP, TRPC4, EDEM2, DDA1 |
| symbols | |

TABLE 13-65
Cluster 65 of the 67 SNP clusters

| | |
|---|---|
| Functional | Diseases associated with O-glycosylation of proteins |
| Annotation | metalloendopeptidase activity |
| | Thrombospondin complex |
| | extracellular structure organization |
| | TGF-beta signaling pathway |
| SNPs | rs12248123, rs4917385, rs352834, rs436872, rs10486135, rs7939069, rs2732159, |
| | rs4777427, rs7712962, rs1059312, rs10773578, rs10847691, rs10847697, rs11059910, |
| | rs11059911, rs11059917, rs11059919, rs11059923, rs11059926, rs11059927, rs11059928, |
| | rs11059930, rs12298615, rs12321679, rs1385374, rs2170988, rs34888374, rs6486730 |
| Gene | SFXN2, ARMC2, THSD7A, MTCH2, ADAMTSL3, THSD4, ADAM19, GLT1D1 |
| symbols | |

TABLE 13-66
Cluster 66 of the 67 SNP clusters

| | |
|---|---|
| Functional | N/A |
| Annotation | |
| SNPs | rs17813747, rs7533588, rs11264697, rs12039812, rs1830847, rs1830848, rs1853689, |
| | rs7197475, rs4921317, rs62262116, rs7625006, rs79821347, rs243323 |
| Gene | REEP3, JMJD4, JMJD1C, PEAR1, KDM8, RNF145, DOCK3, TNP2 |
| symbols | |

TABLE 13-67
Cluster 67 of the 67 SNP clusters

| | |
|---|---|
| Functional | N/A |
| Annotation | |
| SNPs | rs10800816, rs12990918, rs13010752, rs13011805, rs34675442, rs7601754, rs56228409, |
| | rs10905718, rs4748940, rs6088765, rs7031325, rs11539148, rs4625, rs62262116, |
| | rs13389408, rs3024896, rs7568275, rs11741255, rs10774625, rs17630235, rs597808, |
| | rs653178 |
| Gene | PTPN7, MFSD6, NAGLU, PFKFB3, GGT7, GLDC, AMT, GLS, PDLIM4, ALDH2 |
| symbols | |

### REFERENCES

1. Yen E Y, Singh R R. (2018). Brief Report: Lupus—An Unrecognized Leading Cause of Death in Young Females: A Population-Based Study Using Nationwide Death Certificates, 2000-2015. Arthritis Rheumatol. 70(8):1251-5.
2. Liu Y, Kaplan M J. (2018). Cardiovascular disease in systemic lupus erythematosus: An update. Curr Opin Rheumatol. 30(5):441-8.
3. Gupta S, Kaplan MJ. (2021). Bite of the wolf: innate immune responses propagate autoimmunity in lupus. J. Clin. Inves. 131(3): e144918.
4. Thomas G, Mancini J, Jourde-Chiche N, Sarlon G, Amoura Z, Harle J R, Jougla E, Chiche, L. (2014). Mortality associated with systemic lupus erythematosus in France assessed by multiple-cause-of-death analysis. Arthritis Rheumatol. 66(9):2503-11.
5. Teixeira V, Tam LS. (2018). Novel Insights in Systemic Lupus Erythematosus and Atherosclerosis. Front. Med. 4:262.
6. Moe S R, Haukeland H, Molberg O, Lerang K. (2021). Long-term outcome in systemic lupus erythematosus; knowledge from population-based cohorts. J. Clin. Med. 10(19):4306.
7. Katz G, Smilowitz N R, Blazer A, Clancy R, Buyon J P, Berger J S. (2019). Systemic Lupus Erythematosus Is Associated With Increased Prevalence of Atherosclerotic Cardiovascular Disease in Hospitalized Patients. Mayo. Clin. Proc. 94(8):1436.
8. Lopez E O, Ballard B D, Jan A. (2021). Cardiovascular Disease. (2021). In StatPearls [Internet], editorial board. (StatPearls Publishing).
9. Petri M, Perez-Gutthann S, Spence D, Hochberg M C. (1992). Risk factors for coronary artery disease in patients with systemic lupus erythematosus. Am. J. Med. 93(5):513-9.
10. Moghaddam B, Marozoff S, Li L, Sayre E C, Zubieta J A A. (2022). All-cause and cause-specific mortality in systemic lupus erythematosus: a population-based study. Rheumatology. 61(1):367.
11. Bernatsky S, Boivin J F, Joseph L, Manzi S, Ginzler E, Gladman D D, Urowitz M, Fortin P R, Petri M, Barr S, et al. (2006). Mortality in systemic lupus erythematosus. Arthritis. Rheum. 54(8):2550-7.
12. Wu G C, Liu H R, Leng R X, Li X P, Li X M, Pan H F, Ye D-Q. (2016). Subclinical atherosclerosis in patients with systemic lupus erythematosus: A systemic review and meta-analysis. Autoimmun. Rev. 15(1):22-37.
13. Yazdany J, Pooley N, Langham J, Nicholson L, Langham S, Embleton N, Wang X, Desta B, Barut V, Hammond E. (2020). Original research: Systemic lupus erythematosus; stroke and myocardial infarction risk: a systematic review and meta-analysis. RMD Open. 6(2): e00124.
14. Magder L S, Petri M. (2012). Incidence of and Risk Factors for Adverse Cardiovascular Events Among Patients With Systemic Lupus Erythematosus. Am. J. Epidemiol. 176(8):708.
15. Petri M A, Barr E, Magder L S. (2019). Original research: Development of a systemic lupus erythematosus cardiovascular risk equation. Lupus Sci. Med. 6(1):346.
16. Levinson D J, Abugroun A, Daoud H, Abdel-Rahman M. (2020). Coronary artery disease (CAD) risk factor analysis in an age-stratified hospital population with systemic lupus erythematosus (SLE). Int. J. Cardiol. Hypertens. 7: 100056.
17. Ajeganova S, Hafstrom I, Frostegard J. (2021). Patients with SLE have higher risk of cardiovascular events and mortality in comparison with controls with the same levels of traditional risk factors and intima-media measures, which is related to accumulated disease damage and antiphospholipid syndrome: a case-control study over 10 years. Lupus Sci. Med. [Internet]. 8(1): e000454.
18. Bakshi J, Segura B T, Wincup C, Rahman A. Unmet Needs in the Pathogenesis and Treatment of Systemic Lupus Erythematosus. Clin. Rev. Allergy Immunol. 55(3):352-67.
19. Calvo-Alen J, Alarcón G S, Campbell R, Fernandez M, Reveille J D, Cooper G S. (2005). Lack of recording of systemic lupus erythematosus in the death certificates of lupus patients. Rheumatology. 44(9):1186-9.
20. Falasinnu T, Rossides M, Chaichian Y, Simard J F. (2018). Do Death Certificates Underestimate the Burden of Rare Diseases? The Example of Systemic Lupus Erythematosus Mortality, Sweden, 2001-2013. Public Health Rep. 133(4):481-8.
21. Ashley E A, Hershberger R E, Caleshu C, Ellinor P T, Garcia JGN, Herrington D M, Ho C Y, Jhonson J A, Kittner S J, Macrae C A, et al. (2012). Genetics and cardiovascular disease: a policy statement from the American Heart Association. Circulation. 126(1):142-57.
22. Leonard D, Svenungsson E, Dahlqvist J, Alexsson A, Arlestig L, Taylor K E, Sandlin J K, Bengtsson C, Frolund M, Jonsen A, et al. Novel gene variants associated with cardiovascular disease in systemic lupus erythematosus and rheumatoid arthritis. Ann.
   Rheum. Dis. 77(7):1063-9.
23. Ramirez G A. (2018). Genetics in systemic lupus erythematosus: entering the borough of cardiovascular risk. Ann. Transl. Med. 6(Suppl 1):S14.
24. Svenungsson E, Gustafsson J, Leonard D, Sandling J, Gunnarsson I, Nordmark G, Jonsen A, Bengtsson A A, Sturfelt G, Rantapaa-Dahlqvist A, et al. (2010). A STAT4 risk allele is associated with ischaemic cerebrovascular events and anti-phospholipid antibodies in systemic lupus erythematosus. Ann. Rheum. Dis. 69(5):834-40.
25. Leonard D, Svenungsson E, Sandling J K, Berggren O, Jonsen A, Bengtsson C, Wang C, Jensen-Urstad K, Granstam S-O, Bengtsston A A, et al. (2013). Coronary heart disease in systemic lupus erythematosus is associated with interferon regulatory factor-8 gene variants. Circ. Cardiovasc. Genet. 6(3):255-63.
26. Owen K A, Price A, Ainsworth H, Aidukaitis B N, Bachali P, Catalina M D, Dittman J M, Howard T D, Kingsmore K M, Labonte A C, et al. (2021). Analysis of Trans-Ancestral SLE Risk Loci Identifies Unique Biologic Networks and Drug Targets in African and European Ancestries. Am J Hum Genet. 107(5):864-81.
27. Bentham J, Morris D L, Cunninghame Graham D S, Pinder C L, Tombleson P, Behrens T W, Martin J, Fairfax B P, Knight J C, Chen L, Replogle J, et al. (2015). Genetic association analyses implicate aberrant regulation of innate and adaptive immunity genes in the pathogenesis of systemic lupus erythematosus. Nat. Genet. 47(12):1457-64.
28. Lessard C J, Sajuthi S, Zhao J, Kim K, Ice J A, Li H, Ainsworth H, Rasmussen A, Kelly J A, Marion M, et al. (2016). Identification of a Systemic Lupus Erythematosus Risk Locus Spanning ATG16L2, FCHSD2, and P2RY2 in Koreans. Arthritis Rheumatol. 68(5):1197-209.
29. Morris D L, Sheng Y, Zhang Y, Wang Y-F, Zhu Z, Tombleson P, Chen L, Cunninghame Graham D S, Bentham J, Roberts A L, et al. (2016). Genome-wide association meta-analysis in Chinese and European individuals identifies ten new loci associated with systemic lupus erythematosus. Nat. Genet. 48(8):940-6.
30. Sun C, Molineros J E, Looger L L, Zhou X-J, Kim K, Okada Y, Ma J, Qi Y-Y, Howard X K, Motghare P, et al. (2016). High-density genotyping of immune-related loci identifies new SLE risk variants in individuals with Asian ancestry. Nat. Genet. 48(3):323-30.
31. Langefeld C D, Ainsworth H C, Graham DSC, Kelly J A, Comeau M E, Marion M C, Howard T D, Ramos P S, Croker J A, Morris D L, et al. (2017). Transancestral mapping and genetic load in systemic lupus erythematosus. Nat. Commun. 8.:16021.
32. Van Der Harst P, Verweij N. (2018). Identification of 64 novel genetic loci provides an expanded view on the genetic architecture of coronary artery disease. Circ. Res. 122(3):433-43.
33. Finucane H K, Bulik-Sullivan B, Gusev A, Trynka G, Reshef Y, Loh P R, Anttila V, Xu H, Zanh C, Farh K, et al. (2015). Partitioning heritability by functional annotation using genome-wide association summary statistics. Nat. Genet. 47(11):1228-35.
34. Warren H R, Evangelou E, Cabrera C P, Gao H, Ren M, Mifsud B, Ntalla I, Surendran P, Liu C, Cook J P, et al. (2017). Genome-wide association analysis identifies novel blood pressure loci and offers biological insights into cardiovascular risk. Nat. Genet. 49(3):403-15.
35. Schunkert H, Konig I R, Kathiresan S, Reilly M P, Assimes T L, Holm H, Preuss M, Stewart A F R, Barbalic M, Gieger C, et al. (2011). Large-scale association analysis identifies 13 new susceptibility loci for coronary artery disease. Nat. Genet. 43(4):333-40.
36. Howson J M M, Zhao W, Barnes D R, Ho W K, Young R, Paul D S, Waite L L, Freitag D F, Fauman E B, Salfati E L, et al. (2017). Fifteen new risk loci for coronary artery disease highlight arterial-wall-specific mechanisms. Nat. Genet. 49(7):1113-9.
37. Marees A T, de Kluiver H, Stringer S, Vorspan F, Curis E, Marie-Claire C, Derks E M. (2018). A tutorial on conducting genome-wide association studies: Quality control and statistical analysis. Int. J. Methods Psychiatr. Res. 27(2): e1608.
38. Nikpay M, Goel A, Won H H, Hall L M, Willenborg C, Kanoni S, Saleheen D, Kyriakou T, Nelson C P, Hopewell J C, et al. (2015). A comprehensive 1000 Genomes-based genome-wide association meta-analysis of coronary artery disease. Nat. Genet. 47(10):1121-30.
39. Hartiala J A, Han Y, Jia Q, Hilser J R, Huang P, Gukasyan J, Schwartzman W S, Cai Z, Biswas S, Tregouet D-A, et al. (2021). Genome-wide analysis identifies novel susceptibility loci for myocardial infarction. Eur. Heart J. 42(9):919-33.
40. Malik R, Chauhan G, Traylor M, Sargurupremraj M, Okada Y, Mishra A, Rutten-Jacobs L, Giese A-K, van der Laan S W, Gretarsdottir S, et al. (2018). Multiancestry genome-wide association study of 520,000 subjects identifies 32 loci associated with stroke and stroke subtypes. Nat. Genet. 50(4):524-537.
41. Nielsen J B, Thorolfsdottir R B, Fritsche L G, Zhou W, Skov M W, Graham S E, Herron T J, McCarthy S, Schmidt E M, Sveinbjornsson G, et al. (2018). Biobank-driven genomic discovery yields new insight into atrial fibrillation biology. Nat. Genet. 50(9):1234-9.
42. Raj P, Rai E, Song R, Khan S, Wakeland B E, Viswanathan K, Arana C, Liang C, Zhang B, Dozmorov I, et al. (2016). Regulatory polymorphisms modulate the expression of HLA class I I molecules and promote autoimmunity. Elife. 5: e12089.
43. Davies R W, Wells G A, Stewart A F R, Erdmann J, Shah S H, Ferguson J F, Hall A S, Anand S S, Burnett M S, Epstein S E, et al. (2012). A genome-wide association study for coronary artery disease identifies a novel susceptibility locus in the major histocompatibility complex. Circ. Cardiovasc. Genet. 5(2):217-25.
44. Trowsdale J, Knight J C. (2013). Major histocompatibility complex genomics and human disease. Annu. Rev. Genomics Hum. Genet. 14:301-23.
45. Hemani G, Zheng J, Elsworth B, Wade K H, Haberland V, Baird D, Laurin C, Burgess S, Bowden J, Langdon R, et al. (2018). The M R-Base platform supports systematic causal inference across the human phenome. Elife. 7: e34408.
46. Piranavan P, Perl A. (2020). Management of cardiovascular disease in patients with systemic lupus erythematosus. Expert. Opin. Pharmacother. 21(13):1617.
47. Burgess S, Zuber V, Valdes-Marquez E, Sun B B, Hopewell J C. (2017). Mendelian randomization with fine-mapped genetic data: Choosing from large numbers of correlated instrumental variables. Genet. Epidemiol. 41(8):714-25.
48. Reiss A B, Anwar K, Merrill J T, Chan E S L, Awadallah N W, Cronstein B N, Belmont H M, Belilos E, Rosenblum G, Belostocki K, et al. (2010). Plasma from systemic lupus patients compromises cholesterol homeostasis: a potential mechanism linking autoimmunity to atherosclerotic cardiovascular disease. Rheumatol. Int. 30(5):591-8.
49. Quinn C M, Jessup W, Wong J, Kritharides L, Brown A J. (2005). Expression and regulation of sterol 27-hydroxylase (CYP27A1) in human macrophages: a role for RXR and PPARgamma ligands. Biochem. J. 385(Pt 3):823-30.
50. Bilotta M T, Petillo S, Santoni A, Cippitelli M. (2020). Liver X Receptors: Regulators of Cholesterol Metabolism, Inflammation, Autoimmunity, and Cancer. Front. Immunol. 11:584303.
51. Liu A, Rahman M, Hafstrom I, Ajeganova S, Frostegard J. (2020). Proprotein convertase subtilisin kexin 9 is associated with disease activity and is implicated in immune activation in systemic lupus erythematosus. Lupus. 29(8):825-35.
52. Yao Mattisson I, Rattik S, Bjorkbacka H, Ljungcrantz I, Terrinoni M, Lebens M, Holmgren J, Fredrikson G N, Gullstrand B, Bengtsson A A, et al. (2021). Immune responses against oxidized LDL as possible targets for prevention of atherosclerosis in systemic lupus erythematosus. Vascul. Pharmacol. 140:106863.
53. Barnado A, Carroll R J, Casey C, Wheless L, Denny J C, Crofford U. (2018).

Phenome-wide association study identifies marked increased in burden of comorbidities in African Americans with systemic lupus erythematosus. Arthritis Res. Ther. 20(1):69.
54. Esteller M. (2011). Non-coding RNAs in human disease. Nat. Rev. Genet. 12(12):861-74.
55. Hrdlickova B, de Almeida R C, Borek Z, Withoff S. (2014). Genetic variation in the non-coding genome: Involvement of micro-RNAs and long non-coding RNAs in disease. Biochim Biophys Acta. 1842(10):1910-22.
56. The GTEx Consortium (2013). The Genotype-Tissue Expression (GTEx) project. Nat. Gen. 45(6):580-585.
57. Wang J, Dai X, Berry L D, Cogan J D, Liu Q, Shyr Y. (2019). HACER: An atlas of human active enhancers to interpret regulatory variants. Nucleic Acids Res. 47(D1):D106-12.
58. McLaren W, Gil L, Hunt S E, Riat H S, Ritchie G R S, Thormann A, Flicek P, Cunningham F. (2016). The Ensembl Variant Effect Predictor. Genome Biol. 17:122.
59. Szklarczyk D, Gable A L, Nastou K C, Lyon D, Kirsch R, Pyysalo S, Doncheva N T, Legeay M, Fang T, Bork P, et al. (2021). The STRING database in 2021: customizable protein-protein networks, and functional characterization of user-uploaded gene/measurement sets. Nucleic Acids Res. 49(D1):D605-12.
60. Shannon P, Markiel A, Ozier O, Baliga N S, Wang J T, Ramage D, Amin N, Schwikowski B, Ideker T. (2003). Cytoscape: A software Environment for integrated models of biomolecular interaction networks. Genome Res. 13(11):2498-504.
61. Morris J H, Apeltsin L, Newman A M, Baumbach J, Wittkop T, Su G, Bader G D, Ferrin T E. (2011). clusterMaker: a multi-algorithm clustering plugin for Cytoscape. BMC Bioinformatics. 12:436.
62. Labonte A C, Kegerreis B, Geraci N S, Bachali P, Madamanchi S, Robl R, Catalina M D, Lipsky P E, Grammer A C. (2018). Identification of alterations in macrophage activation associated with disease activity in systemic lupus erythematosus. PLoS One. 13(12): e0208132.
63. Catalina M D, Bachali P, Geraci N S, Grammer A C, Lipsky P E. (2019). Gene expression analysis delineates the potential roles of multiple interferons in systemic lupus erythematosus. Commun. Biol. 2019 December; 2(1).
64. Catalina M D, Owen K A, Labonte A C, Grammer A C, Lipsky P E. (2019). The pathogenesis of systemic lupus erythematosus: Harnessing big data to understand the molecular basis of lupus. J. Autoimmun. 110:102359.
65. Ren J, Catalina M D, Eden K, Liao X, Read K A, Luo X, McMillan R P, Hulver M W, Jarpe M, Bachali P, Grammer A C, et al. (2019). Selective histone deacetylase 6 inhibition normalizes b cell activation and germinal center formation in a model of systemic lupus erythematosus. Front. Immunol. 10:2512.
66. Chen E Y, Tan C M, Kou Y, Duan Q, Wang Z, Meirelles G V, Clark N R, Ma'ayan A. (2013).Enrichr: interactive and collaborative HTML5 gene list enrichment analysis tool. BMC Bioinformatics. 14:128.
67. Yang W, Shen N, Ye D Q, Liu Q, Zhang Y, Qian X X, Hirankarn N, Ying D, Pan H-F, Mok C C, et al. (2010). Genome-wide association study in Asian populations identifies variants in ETS1 and WDFY4 associated with systemic lupus erythematosus. PLoS Genet. 6(2): e1000841.
68. Chung S A, Taylor K E, Graham R R, Nititham J, Lee A T, Ortmann W A, Jacob C O, Alarcon-Riquelme M E, Tsao B P, Harley J B, et al. (2011). Differential genetic associations for systemic lupus erythematosus based on anti-dsDNA autoantibody production. PLoS Genet. 7(3): e1001323.
69. Yang J, Yang W, Hirankam N, Ye D Q, Zhang Y, Pan H F, Mok C C, Chan T M, Wong R W S, Mok M Y, et al. (2011). ELF1 is associated with systemic lupus erythematosus in Asian populations. Hum. Mol. Genet. 20(3):601-7.
70. Lee Y H, Bae S C, Choi S J, Ji J D, Song G G. (2012). Genome-wide pathway analysis of genome-wide association studies on systemic lupus erythematosus and rheumatoid arthritis. Mol. Biol. Rep. 39(12):10627-35.
71. Okada Y, Shimane K, Kochi Y, Tahira T, Suzuki A, Higasa K, Takahashi A, Horita T, Atsumi T, Ishii T, et al. (2012). A genome-wide association study identified AFF1 as a susceptibility locus for systemic lupus eyrthematosus in Japanese. PLoS Genet. 8(1): e1002455.
72. Yang W, Tang H, Zhang Y, Tang X, Zhang J, Sun L, Yang J, Cui Y, Zhang L, Hirankarn N, et al. (2013). Meta-analysis followed by replication identifies loci in or near CDKN1B, TET3, CD80, DRAM1, and ARID5B as associated with systemic lupus erythematosus in Asians. Am. J. Hum. Genet. 92(1):41-51.
73. Armstrong D L, Zidovetzki R, Alarcón-Riquelme M E, Tsao B P, Criswell L A, Kimberly R P, Harley J B, Sivils K L, Vyse T J, Gaffnet P M, et al. (2014). GWAS identifies novel SLE susceptibility genes and explains the association of the HLA region. Genes Immun. 15(6):347-54.
74. Alarcón-Riquelme M E, Ziegler J T, Molineros J, Howard T D, Moreno-Estrada A, Sinchez-Rodriguez E, Ainsworth H C, Ortiz-Tello P, Comeau M E, Rasmussen A, et al. (2016). Genome-Wide Association Study in an Amerindian Ancestry Population Reveals Novel Systemic Lupus Erythematosus Risk Loci and the Role of European Admixture. Arthritis Rheumatol. 68(4):932-43.
75. Hom G, Graham R R, Modrek B, Taylor K E, Ortmann W, Gamier S, Lee A T, Chung S A, Ferreira R C, Pant P V K, et al. (2008). Association of systemic lupus erythematosus with C8orf13-BLK and ITGAM-ITGAX. N. Engl. J. Med. 358(9):900-9.
76. Graham R R, Cotsapas C, Davies L, Hackett R, Lessard C J, Leon J M, Burtt N P, Guiducci C, Parkin M, Gates C, Plenge R M, et al. (2008). Genetic variants near TNFAIP3 on 6q23 are associated with systemic lupus erythematosus. Nat. Genet. 40(9):1059-61.
77. Harley J B, Alarcón-Riquelme M E, Criswell L A, Jacob C O, Kimberly R P, Moser K L, Tsao B P, Vyse T J, Langefeld C D, Nath S, et al. (2008). Genome-wide association scan in women with systemic lupus erythematosus identifies susceptibility variants in ITGAM, PXK, KIAA1542 and other loci. Nat. Genet. 40(2):204-10.
78. Kozyrev S V., Abelson A K, Wojcik J, Zaghlool A, Linga Reddy MVP, Sanchez E, Gunnarsson I, Svenungsson E, Sturfelt G, Jonsen A, et al. (2008). Functional variants in the B-cell gene BANK1 are associated with systemic lupus erythematosus. Nat. Genet. 40(2):211-6.
79. Oishi T, Iida A, Otsubo S, Kamatani Y, Usami M, Takei T, Uchida K, Tsuchiya K, Saito S, Ohnisi Y, et al. (2008). A functional SNP in the NKX2.5-binding site of ITPR3 promoter is associated with susceptibility to systemic lupus erythematosus in Japanese population. J. Hum. Genet. 53(2):151-62.
80. Gateva V, Sandling J K, Hom G, Taylor K E, Chung S A, Sun X, Ortmann W, Kosoy R, Ferreira R C, Nordmark G, et al. (2009). A large-scale replication study identifies TNIP1, PRDM1, JAZF1, UHRF1BP1 and IL10 as risk loci for systemic lupus erythematosus. Nat. Genet. 41(11):1228-33.
81. Han J W, Zheng H F, Cui Y, Sun L D, Ye D Q, Hu Z, Xu J-H, Cai Z-M, Huang W, Zhao G-P, et al. (2009). Genome-wide association study in a Chinese Han population identifies nine new susceptibility loci for systemic lupus erythematosus. Nat. Genet. 41(11):1234-7.
82. Staley J R, Blackshaw J, Kamat M A, Ellis S, Surendran P, Sun B B, Paul D S, Freitag D, Burgess S, Danesh J, et al. (2016). PhenoScanner: a database of human genotype-phenotype associations. Bioinformatics. 32(20):3207-9.
83. Kamat M A, Blackshaw J A, Young R, Surendran P, Burgess S, Danesh J, Butterworth A S, Staley J R. (2019). PhenoScanner V2: an expanded tool for searching human genotype-phenotype associations. Bioinformatics. 35(22):4851-3.
84. Keenan A B, Jenkins S L, Jagodnik K M, Koplev S, He E, Torre D, Wang Z, Dohlman, Silverstein M C, Lachmann A, et al. (2018). The Library of Integrated Network-Based Cellular Signatures NIH Program: System-Level Cataloging of Human Cells Response to Perturbations. Cell Syst. 6(1):13-24.
85. Kuhn M, Szklarczyk D, Franceschini A, Campillos M, Von Mering C, Jensen L J, Beyer A, Bork P. (2010). STITCH 2: an interaction network database for small molecules and proteins. Nucleic Acids Res. 38(Database issue):D552-6.
86. Demirci F Y, Wang X, Kelly J A, Morris D L, Barmada M M, Feingold E. Kao A H, Sivils K L, Bernatsky S, Pineau C, et al. (2016). Identification of a new susceptibility locus for systemic lupus erythematosus on chromosome 12 in individuals of European ancestry. Arthritis Rheumatol. 68(1)174-83.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It may be understood that various alternatives to the embodiments described herein may be employed in practice. Numerous different combinations of embodiments described herein are possible, and such combinations are considered part of the present disclosure. In addition, all features discussed in connection with any one embodiment herein may be readily adapted for use in other embodiments herein. It is intended that the following claims define the scope of the disclosure and that methods and structures within the scope of these claims and their equivalents be covered thereby.

1. A method for determining shared biological pathways, and shared nucleotide polymorphisms (NPs), between a first disease and a second disease, the method comprising:
(a) selecting a first set of nucleotide polymorphisms (NPs) associated with the first disease from a first dataset, wherein the first dataset comprises data regarding association of a first plurality of NPs with the first disease;
(b) mapping one or more NPs of the first set of NPs to genes, to identify a plurality of NP-mapped genes;
(c) clustering the plurality of NP-mapped genes to obtain one or more gene clusters;
(d) clustering of the one or more NPs mapped in (b) to obtain a first set of NP clusters;
(e) performing a causal inference analysis to select a subset of NP clusters from the first set of NP clusters obtained in (d), wherein each NP cluster within the subset of NP clusters has a positive or negative causal effect on the second disease; and
(f) functionally annotating i) one or more NP clusters of the subset of NP clusters selected in (e), and/or ii) gene clusters mapped with the one or more NP clusters of the subset of NP clusters, thereby determining the shared biological pathways between the first disease and the second disease,
wherein the NPs within the NP clusters within the subset of NP clusters selected in (e) are the shared NPs between the first and second disease.

2. The method of claim 1, wherein the clustering of the one or more NPs mapped in (b) to obtain the first set of NP clusters in (d), is performed based on the clustering of the plurality of NP-mapped genes in (c).

3. The method of claim 1, wherein a p-value for statistical significance of the association of each NP in the first set of NPs with the first disease is lower than 1*10⁻⁶ or lower than 5*10⁻⁸.

4. (canceled)

5. The method of claim 1, wherein in (b) the plurality of NP-mapped genes are identified by mapping the one or more NPs of the first set of NPs independently to their i) associated expression quantitative trait loci (eQTL) expression genes (E-Genes), ii) associated transcription factors and downstream target genes (T-Genes), iii) associated protein coding genes (C-genes), and/or iv) proximal genes (P-genes).

6. The method of claim 1, wherein in (c) the plurality of NP-mapped genes are clustered based on protein-protein interactions of proteins encoded by the plurality of NP-mapped genes, gene co-expression, genetic pathway, genetic annotations, genetic associations, or any combination thereof.

7. (canceled)

8. The method of claim 1, wherein clustering the plurality of NP-mapped genes comprises:
clustering the encoded proteins into one or more protein clusters, wherein proteins determined to be within same biological pathway are grouped into same protein cluster; and
clustering the plurality of NP-mapped genes to obtain one or more gene clusters of (c), based at least on clustering of the encoded proteins, wherein for a respective protein cluster formed, a gene cluster is formed containing the genes that encodes the proteins within the respective protein cluster.

9. The method of claim 1, wherein the causal inference analysis is a mendelian randomization (MR) based method.

10. The method of claim 9, wherein the MR based method comprises determining a causal effect of the NP clusters of the first set of NP clusters on the second disease, wherein for a respective NP cluster, at least 2 NPs within the cluster collectively is used as instrument variables, summary statistics from a second dataset is used as exposure, and a third dataset is used as outcome, wherein the second dataset comprises the summary statistics regarding association of a second plurality of NPs with the first disease, and the third dataset comprises data regarding association of a third plurality of NPs with the second disease.

11. The method of claim 10, wherein in the MR based method, for a respective NP cluster of the first set of NP clusters the NPs used as instrument variables are selected based on: (i) the strength of association of the NPs with the first disease; (ii) the strength of association of the NPs with the second disease, (iii) the strength of association of NPs with confounding traits; (iv) linkage disequilibrium with other NPs; (v) genomic location; (vi) allele harmonization between the second and third datasets; or any combination thereof.

12. The method claim 1, wherein for a respective NP cluster of the subset of NP clusters selected in (e), the functionally annotating comprises:
(i) overlapping a gene cluster mapped with the respective NP cluster, with one or more gene function signature lists to determine, significant overlap between the gene cluster and the one or more gene function signature lists; and
(ii) annotating the respective NP cluster with one or more functional characterizations, based at least on the significant overlap.

13. The method of claim 1, wherein the first disease is selected from lupus, coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder, inflammatory bowel disease, multiple sclerosis, and glomerulonephritis.

14. (canceled)

15. The method of claim 1, wherein the second disease is different from the first disease and is selected from lupus, cardiovascular disease, CAD, myocardial infarction, ischemic stroke, coronary atherosclerosis, cardiomyopathy, depression, asthma, COPD, diabetes mellitus, nonalcoholic fatty liver disease, metabolic disorder, inflammatory bowel disease, multiple sclerosis, and glomerulonephritis.

16. (canceled)

17. The method of claim 1, wherein the NPs are single nucleotide polymorphisms (SNPs), indels, or splice variants.

18. (canceled)

19. A method for determining shared biological pathways, and shared single nucleotide polymorphisms (SNPs) between lupus and a second disease, the method comprising:
(a) selecting a first set of SNPs associated with lupus from a first dataset, wherein the first dataset comprises data regarding association of a first plurality of SNPs with lupus;
(b) mapping one or more SNPs of the first set of SNPs selected in (a), to genes to identify a plurality of SNP-mapped genes;
(c) clustering the plurality of SNP-mapped genes based on protein-protein interaction of proteins encoded by the SNP-mapped genes to obtain one or more gene clusters;
(d) clustering of the one or more SNP mapped in (b) to obtain a first set of SNP clusters, based on clustering of the plurality of SNP-mapped genes in (c);
(e) performing a Mendelian randomization (MR) based method to select a subset of SNP clusters from the first set of SNP clusters obtained in (d), wherein each SNP cluster within the subset of SNP clusters independently has a positive or negative causal effect on the second disease; and
(f) functionally annotating i) one or more SNP clusters of the subset of SNP clusters selected in (e), and/or ii) gene clusters mapped with the one or more SNP clusters of the subset of SNP clusters, thereby determining the shared biological pathways between lupus and the second disease,
wherein the SNPs in the SNP clusters within the subset of SNP clusters obtained in (e) are the shared SNPs between lupus and the second disease.

20. The method of claim 19, wherein a p-value for statistical significance of the association of each SNP in of the first set of SNPs with lupus is lower than about 1*10⁻⁴, lower than about 5*10⁻⁵, lower than about 1*10⁻⁵, lower than about 5*10⁻⁶, lower than about 1*10⁻⁶, lower than about 5*10⁻⁷, lower than about 1*10⁻⁷, lower than about 5*10⁻⁸, or lower than about 1*10⁻⁸.

21. (canceled)

22. (canceled)

23. The method of claim 19, wherein in (b) the plurality of SNP-mapped genes are identified by mapping one or more SNPs of the subset of SNPs independently to their i) associated expression quantitative trait loci (eQTL) expression genes (E-Genes), ii) associated transcription factors and downstream target genes (T-Genes), iii) associated protein coding genes (C-genes), and/or iv) proximal genes (P-genes).

24. The method of claim 19, wherein in (c) the clustering of the plurality of SNP-mapped genes based on protein-protein interaction comprises:
clustering the encoded proteins into one or more protein clusters, wherein proteins determined to be within same biological pathway are grouped into same protein cluster; and
clustering the SNP-mapped genes to form the one or more gene clusters of (c), based at least on clustering of the encoded proteins, wherein for a respective protein cluster formed, a gene cluster is formed containing the genes that encodes the proteins within the respective protein cluster.

25. The method of claim 19, wherein the MR based method in (e) comprises determining a causal effect of the SNP clusters of the first set of SNP clusters on the second disease, wherein for a respective SNP cluster, at least 2 SNPs within the cluster, collectively is used as instrument variables, summary statistics from a second dataset is used as exposure, and a third dataset is used as outcome, wherein the second dataset comprises the summary statistics regarding association of a second plurality of SNPs with lupus, and the third dataset comprises data regarding association of a third plurality of SNPs with the second disease.

26. The method of claim 25, wherein in the MR based method of (e) independently for each SNP cluster of the first set of SNP clusters SNPs used as instrument variables are selected based on: (i) the strength of association of the SNPs with lupus; (ii) the strength of association of the SNPs with the second disease; (iii) the strength of association of SNPs with confounding traits; (iv) linkage disequilibrium with other SNPs; (v) genomic location; (vi) allele harmonization between the second and third datasets; or any combination thereof.

27. The method of claim 19, wherein for a respective SNP cluster of the subset of SNP clusters selected in (e), the functionally annotating comprises:
(i) overlapping a gene cluster mapped with the respective SNP cluster, with one or more gene function signature lists to determine, significant overlap between the gene cluster and the one or more gene function signature lists; and
(ii) annotating the respective SNP cluster with one or more functional characterizations, based at least on the significant overlap.

28. The method of claim 19, wherein the second disease is selected from coronary artery disease (CAD), cardiovascular disease, myocardial infarction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, and glomerulonephritis.

29. (canceled)

30. A method for determining a coronary artery disease (CAD) state in a patient, the method comprising:
detecting one or more SNPs selected from SNPs listed in Tables: 13-1; 13-2; 13-3; 13-4; 13-5; 13-6; 13-7; 13-8; 13-9; 13-10; 13-11; 13-12; 13-13; 13-14; 13-15; 13-16; 13-17; 13-18; 13-19; 13-20; 13-21; 13-22; 13-23; 13-24; 13-25; 13-26; 13-27; 13-28; 13-29; 13-30; 13-31; 13-32; 13-33; 13-34; 13-35; 13-36; 13-37; 13-38; 13-39; 13-40; 13-41; 13-42; 13-43; 13-44; 13-45; 13-46; 13-47; 13-48; 13-49; 13-50; 13-51; 13-52; 13-53; 13-54; 13-55; 13-56; 13-57; 13-58; 13-59; 13-60; 13-61; 13-62; 13-63; 13-64; 13-65; 13-66; and 13-67; in a biological sample from the patient; and
determining the CAD state in the patient, based at least on the presence of the one or more SNPs in the biological sample.

31. The method of claim 30, wherein;_(i) the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6: or (ii) the one or more SNPs are selected from the SNPs listed in Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67.

32. (canceled)

33. The method of claim 30, wherein:
(i) the one or more SNPs comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390, 400, 410, 420, 430, 440, 450, 460, 470, 480, 490, 500, 600, 700, 800, 900, 1000, 1500, 2000, 2500, 3000, 3500, 4000, or 4451 SNPs;
(ii) the one or more SNPs comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300, or all SNPs, selected from the SNPs listed in each of one or more Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; 13-67; 13-10; 13-24; 13-25; 13-30; 13-40; 13-45; 13-50; 13-61; 13-66; 13-1; and 13-6, wherein the number of SNPs selected from different Tables are same or different: or
(iii) the one or more SNPs comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300, or all SNPs, selected from the SNPs listed in each of one or more Tables selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67, wherein the number of SNPs selected from different Tables are same or different.

34. (canceled)

35. (canceled)

36. The method of claim 30, wherein the presence of the one or more SNPs in the biological sample is detected by analyzing nucleic acid of the patient in the biological sample.

37. The method of claim 30, wherein the biological sample is a blood sample, isolated peripheral blood mononuclear cells (PBMCs), tissue biopsy sample, nasal fluid, saliva, urine, stool, or any derivative thereof.

38. The method of claim 30, wherein a disease risk score is calculated based at least on the presence of the one or more SNPs in the biological sample, and the CAD state in the patient is determined based at least on the disease risk score.

39. The method of claim 30, wherein the patient: (i) has lupus; (ii) is at an elevated risk of having lupus; (iii) does not have lupus; or (iv) is asymptomatic for lupus.

40. (canceled)

41. (canceled)

42. (canceled)

43. The method of claim 30, further comprising administering a treatment to the patient based on the determined CAD state.

44. The method of claim 43, wherein the treatment administered is based at least on functional annotation of at least one Table selected from Tables: 13-2; 13-3; 13-4; 13-5; 13-8; 13-12; 13-13; 13-14; 13-15; 13-18; 13-19; 13-21; 13-23; 13-28; 13-31; 13-33; 13-34; 13-36; 13-43; 13-44; 13-48; 13-51; 13-55; 13-60; 13-65; and 13-67; wherein one or more SNPs selected from the SNPs listed in the at least one Table, are present in the biological sample.

45. The method of claim 43, wherein the treatment: (i) is configured to treat CAD; (ii) is configured to reduce severity of CAD; (iii) is configured to reduce a risk of developing CAD; or (iv) comprises a treatment for atherosclerosis.

46. (canceled)

47. (canceled)

48. (canceled)

49. The method of claim 43, wherein the treatment comprises anti-IFN antibodies, anti-oxidized LDL antibodies, anti-PCSK9 antibodies, a JAK inhibitor, a MTOR inhibitor, a MPO inhibitor, an ACE inhibitor, statins, or any combination thereof, optionally wherein the treatment comprises a pharmaceutical composition.

50. (canceled)
